(19) United States
(12) Patent Application Publication
(10) Pub. No.: US 2024/0366598 A1
(43) Pub. Date: Nov. 7, 2024

(54) GLUCOSE-DEPENDENT INSULINOTROPIC POLYPEPTIDE RECEPTOR ANTAGONISTS AND USES THEREOF

(71) Applicant: Pfizer Inc., New York, NY (US)

(72) Inventors: Kevin James Filipski, Reading, MA (US); Matthew Forrest Sammons, Quincy, MA (US); Samuel Michael Levi, Medford, MA (US); Yang Wang, West Newton, MA (US); Advaita Panchagnula, Mystic, CT (US); Yanfei Guan, Arlington, MA (US); Matthew Richard Reese, Mystic, CT (US); Luis Angel Martinez Alsina, Gales Ferry, CT (US); Steven Victor O'Neil, East Lyme, CT (US); Lei Zhang, Auburndale, MA (US); Qifang Li, Stonington, CT (US); Ethan Lawrence Fisher, Chester, CT (US); Danica Antonia Rankic, East Lyme, CT (US)

(73) Assignee: Pfizer Inc., New York, NY (US)

(21) Appl. No.: 18/634,154

(22) Filed: Apr. 12, 2024

Related U.S. Application Data
(60) Provisional application No. 63/496,232, filed on Apr. 14, 2023.
(60) Provisional application No. 63/598,698, filed on Nov. 14, 2023.

(51) Int. Cl. A61K 31/506; A61K 31/403; A61K 31/4439; A61K 31/444; A61P 3/04; C07D 207/16; C07D 209/52; C07D 401/12; C07D 401/14; C07D 403/12

(52) U.S. Cl. CPC A61K 31/506; A61K 31/403; A61K 31/4439; A61K 31/444; A61P 3/04; C07D 207/16; C07D 209/52; C07D 401/12; C07D 401/14; C07D 403/12

(57) ABSTRACT

Described herein are compounds of Formula I:

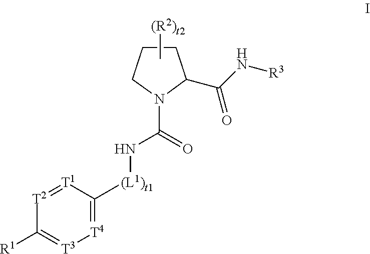

and their pharmaceutically acceptable salts, wherein R¹, R², R³, L¹, T¹, T², T³, T⁴, t1, and t2 are defined herein; their use as GIPR antagonists; pharmaceutical compositions containing such compounds and salts; and the use of such compounds and salts to treat or prevent, for example, obesity, weight gain, and/or T2DM.

This application claims the benefit of priority to U.S. Provisional Patent Application Ser. No. 63/496,232 filed Apr. 14, 2023 and to U.S. Provisional Patent Application Ser. No. 63/598,698 filed Nov. 14, 2023, the disclosure of each of which is hereby incorporated by reference in its entirety.

### FIELD OF THE INVENTION

The present invention relates to new pharmaceutical compounds, pharmaceutical compositions containing the compounds, and use of the compounds as glucose-dependent insulinotropic polypeptide receptor (GIPR) antagonists.

### BACKGROUND OF THE INVENTION

Glucose-dependent insulinotropic polypeptide (GIP, formerly called gastric inhibitory polypeptide) is a 42-amino acid peptide secreted from K-cells in the small intestine (duodenum and jejunum). Human GIP is derived from the processing of proGIP, a 153-amino acid precursor encoded by a gene localized on chromosome 17 (See e.g., Inagaki et al., Mol Endocrinol 1989; 3:1014-1021; and Fehmann et al. Endocr Rev. 1995; 16:390-410). GIP secretion is induced by food ingestion. GIP is a known insulinotropic factor (or “incretin”) that enhances glucose-dependent insulin secretion. GIP has additional physiological effects in multiple tissues, including the promotion of fat storage in the adipose. Intact GIP is rapidly inactivated by dipeptidyl peptidase 4 (DPPIV).

The GIP receptor (GIPR) belongs to the glucagon subfamily of class 1 G protein-coupled receptors (GPCRs) characterized by an extracellular N-terminal domain, seven transmembrane domains and an intracellular C-terminus (See e.g. Zhao et al. Nat Commun. 2022, 13:1057). The N-terminal extracellular domain forms the primary peptide recognition and binding site of the receptor. Upon stimulation with GIP, GIPR undergoes structural changes from inactive to active conformations, thereby triggering a Gas-mediated increase in cAMP production. GIPR is expressed in various tissues, including the pancreas, gut, adipose tissue, vasculature, heart, and brain (see e.g. Hammoud et al. Nat Rev Endocrinol 2023; 18: 201-216). Human GIPR comprises 466 amino acids and is encoded by a gene located on chromosome 19 (see e.g. Gremlich et al., Diabetes. 1995; 44:1202-8; and Volz et al., FEBS Lett. 1995, 373:23-29). Studies suggest that alternative mRNA splicing results in the production of GIPR variants with differing length (see e.g., Harada et al. Am J Physiol Endocrinol Metab. 2008. 294: E61-E68; and Marti-Solano et al. Nature. 2020, 587: 650-656).

GIPR knockout mice are resistant to high fat diet-induced weight gain and have improved insulin sensitivity and lipid profiles (see e.g. Yamada et al. Diabetes. 2006, 55:S86; and Miyawaki et al. Nature Med. 2002, 8:738-742). Recent data supports that heterozygous loss of function in GIPR results in lower BMI and obesity risk in humans (see e.g. Akbari et al. Science. 2021, 373: 6550). Small molecules, peptides, and monoclonal antibodies with antagonist activity at GIPR have been shown to prevent weight gain and insulin resistance in preclinical obesity models (see e.g. Nakamura et al. Diabetes Metab Syndr Obes. 2021,14:1095-1105; Yang et al. Mol Metab. 2022, 66: 101638; and Killion et al. Sci. Transl. Med., 2018, 10:eaat3392). The combination of GIPR modulators with GLP-1R agonists has been associated with superior weight loss (see e.g. Lu et al. Cell Rep Med. 2021, 2(5):100263). Collectively, these links to obesity and metabolic diseases suggest that GIPR inhibition is a useful approach for therapeutic intervention, both as monotherapy and in combination with other agents including GLP-1R agonists. Moreover, human epicardial adipose tissue—which plays a crucial role in the development and progression of coronary artery disease, atrial fibrillation, and heart failure—has been found to express GIPR genes and proteins. See e.g. Malavazos et al., European Journal of Preventive Cardiology (2023) 00, 1-14.

There continues to be a need for alternative GIPR antagonists, for example, for developing new and/or improved pharmaceuticals (e.g., more effective, more selective, less toxic, improved patient compliance, and/or having improved biopharmaceutical properties such as physical stability; solubility; oral bioavailability; appropriate metabolic stability; clearance; half life) to treat or prevent GIPR-related conditions, diseases, or disorders, such as those described herein. The present invention is directed to these and other important ends.

### SUMMARY OF THE INVENTION

In one embodiment (Embodiment A1), the present invention provides a compound of Formula I:

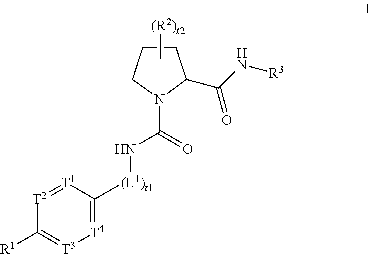

or a pharmaceutically acceptable salt thereof, wherein:
R¹ is H, halogen, —CN, C₁₋₈ alkyl, C₂₋₈ alkenyl, (C₃₋₆ cycloalkyl)-C₁₋₄ alkyl-, or C₃₋₆ cycloalkyl, wherein each of the C₁₋₈ alkyl, C₂₋₈ alkenyl, (C₃₋₆ cycloalkyl)-C₁₋₄ alkyl-, or C₃₋₆ cycloalkyl is optionally substituted with 1, 2, 3, 4, 5, or 6 substituents each independently selected from halogen, —OH, —CN, C₁₋₄ alkyl, C₁₋₄ haloalkyl, C₁₋₄ alkoxy, and C₁₋₄ haloalkoxy;
each R² is independently halogen, —OH, C₁₋₄ alkyl, C₁₋₄ haloalkyl, C₁₋₄ alkoxy, C₁₋₄ haloalkoxy, C₃₋₄ cycloalkyl, or (C₃₋₄ cycloalkyl)-C₁₋₄ alkyl-, wherein each of the C₁₋₄ alkyl, C₁₋₄ haloalkyl, C₁₋₄ alkoxy, C₁₋₄ haloalkoxy, C₃₋₄ cycloalkyl, or (C₃₋₄ cycloalkyl)-C₁₋₄ alkyl- is optionally substituted with 1, 2, or 3 substituents each independently selected from halogen, —OH, C₁₋₄ alkyl, C₁₋₄ haloalkyl, C₁₋₄ alkoxy, and C₁₋₄ haloalkoxy;
or two R², when attached to a same ring carbon atom of the proline ring in Formula I, together with the ring carbon atom to which they are attached, optionally form C₃₋₆ cycloalkyl or a 4-to 7-membered heterocycloalkyl, each of which is optionally substituted with 1, 2, 3, or 4 substituents each independently selected from halogen, —OH, C₁₋₄ alkyl, C₁₋₄ haloalkyl, C₁₋₄ alkoxy, and C₁₋₄ haloalkoxy;
or two R², when attached to two adjacent ring carbon atoms of the proline ring in Formula I, together with the two ring carbon atoms to which they are attached, optionally form C₃₋₆ cycloalkyl or a 4- to 7-membered heterocycloalkyl, each of which is optionally substituted with 1, 2, 3, or 4 substituents each independently selected from halogen, —OH, C₁₋₄ alkyl, C₁₋₄ haloalkyl, C₁₋₄ alkoxy, and C₁₋₄ haloalkoxy;
R³ is R^{3a}, R^{3b}, R^{3c} or R^{3d}:

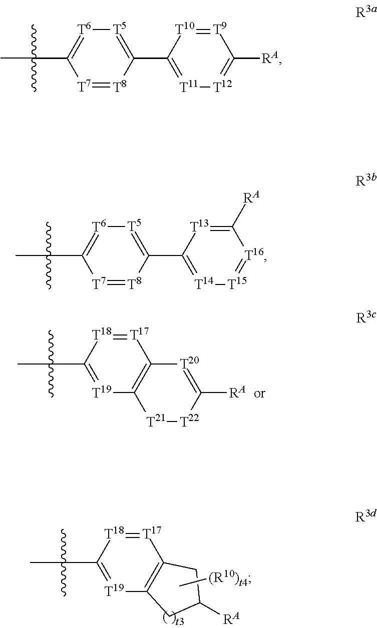

each of T¹, T², T³, and T⁴ is independently CR⁴ or N, provided that only 0, 1, or 2 of T¹, T², T³, and T⁴ can be N;
each R⁴ is independently H, halogen, —CN, C₃₋₆ cycloalkyl, (C₃₋₆ cycloalkyl)-C₁₋₂ alkyl-, C₁₋₄ alkyl, C₁₋₄ cyanoalkyl, C₁₋₄ haloalkyl, C₁₋₄ alkoxy, or C₁₋₄ haloalkoxy;
each of T⁵, T⁶, T⁷, and T⁸ is independently CR⁵ or N, provided that only 0, 1, or 2 of T⁵, T⁶, T⁷, and T⁸ can be N;
each R⁵ is independently H, halogen, —CN, C₃₋₆ cycloalkyl, (C₃₋₆ cycloalkyl)-C₁₋₂ alkyl-, C₁₋₄ alkyl, C₁₋₄ cyanoalkyl, C₁₋₄ haloalkyl, C₁₋₄ alkoxy, or C₁₋₄ haloalkoxy; each of T⁹, T¹⁰, T¹¹, and T¹² is independently CR⁶ or N, provided that only 0, 1, or 2 of T⁹, T¹⁰, T¹¹, and T¹² can be N;
each R⁶ is independently H, halogen, —CN, C₃₋₆ cycloalkyl, (C₃₋₆ cycloalkyl)-C₁₋₂ alkyl-, C₁₋₄ alkyl, C₁₋₄ cyanoalkyl, C₁₋₄ haloalkyl, C₁₋₄ alkoxy, or C₁₋₄ haloalkoxy; each of T¹³, T¹⁴, T¹⁵, and T¹⁶ is independently CR⁷ or N, provided that only 0, 1, or 2 of T¹³, T¹⁴, T¹⁵, and T¹⁶ can be N;
each R⁷ is independently H, halogen, —CN, C₃₋₆ cycloalkyl, (C₃₋₆ cycloalkyl)-C₁₋₂ alkyl-, C₁₋₄ alkyl, C₁₋₄ cyanoalkyl, C₁₋₄ haloalkyl, C₁₋₄ alkoxy, or C₁₋₄ haloalkoxy;
each of T¹⁷, T¹⁸, and T¹⁹ is independently CR⁸ or N, provided that only 0, 1, or 2 of T¹⁷, T¹⁸, and T¹⁹ can be N;
each R⁸ is independently H, halogen, —CN, C₃₋₆ cycloalkyl, (C₃₋₆ cycloalkyl)-C₁₋₂ alkyl-, C₁₋₄ alkyl, C₁₋₄ cyanoalkyl, C₁₋₄ haloalkyl, C₁₋₄ alkoxy, or C₁₋₄ haloalkoxy;
each of T²⁰, T²¹, and T²² is independently CR⁹ or N, provided that only 0, 1, or 2 of T²⁰, T²¹, and T²² can be N;
each R⁹ is independently H, halogen, —CN, C₃₋₆ cycloalkyl, (C₃₋₆ cycloalkyl)-C₁₋₂ alkyl-, C₁₋₄ alkyl, C₁₋₄ cyanoalkyl, C₁₋₄ haloalkyl, C₁₋₄ alkoxy, or C₁₋₄ haloalkoxy;
each R¹⁰ is independently halogen, —OH, C₁₋₄ alkyl, C₁₋₄ haloalkyl, C₁₋₄ alkoxy, C₁₋₄ haloalkoxy, C₃₋₄ cycloalkyl, or (C₃₋₄ cycloalkyl)-C₁₋₄ alkyl-, wherein each of the C₁₋₄ alkyl, C₁₋₄ haloalkyl, C₁₋₄ alkoxy, C₁₋₄ haloalkoxy, C₃₋₄ cycloalkyl, or (C₃₋₄ cycloalkyl)-C₁₋₄ alkyl- is optionally substituted with 1, 2, or 3 substituents each independently selected from halogen, —OH, C₁₋₄ alkyl, C₁₋₄ haloalkyl, C₁₋₄ alkoxy, and C₁₋₄ haloalkoxy;
R^{A} is —C(═O)—OH, 1H-tetrazol-5-yl, OH, —C(═O)—N(R¹¹)(R¹²), —C(═O)—OR¹³, 3-hydroxyisoxazol-5-yl, or —S(═O)₂NHCF₃;
each of R¹¹ and R¹² is independently H, C₁₋₆ alkyl, C₃₋₆ cycloalkyl, (C₃₋₆ cycloalkyl)-C₁₋₄ alkyl-, phenyl, or phenyl-C₁₋₄ alkyl-, wherein each of the C₁₋₆ alkyl, C₃₋₆ cycloalkyl, (C₃₋₆ cycloalkyl)-C₁₋₄ alkyl-, phenyl, or phenyl-C₁₋₄ alkyl- is optionally substituted with 1, 2, 3, 4, or 5 substituents each independently selected from halogen, —OH, —CN, C₁₋₄ alkyl, C₁₋₄ haloalkyl, C₁₋₄ alkoxy, C₁₋₄ haloalkoxy, C₃₋₄ cycloalkyl, or (C₃₋₄ cycloalkyl)-C₁₋₄ alkyl-;
or R¹¹ and R¹² together with the nitrogen atom to which they are attached form a 4- to 8-membered heterocycloalkyl optionally substituted with 1, 2, 3, 4, or 5 substituents each independently selected from halogen, —OH, —CN, C₁₋₄ alkyl, C₁₋₄ haloalkyl, C₁₋₄ alkoxy, C₁₋₄ haloalkoxy, C₃₋₄ cycloalkyl, or (C₃₋₄ cycloalkyl)-C₁₋₄ alkyl-, wherein each of the C₁₋₄ alkyl, C₁₋₄ haloalkyl, C₁₋₄ alkoxy, C₁₋₄ haloalkoxy, C₃₋₄ cycloalkyl, or (C₃₋₄ cycloalkyl)-C₁₋₄ alkyl- is optionally substituted with 1, 2, or 3 substituents each independently selected from halogen, —OH, C₁₋₄ alkyl, C₁₋₄ haloalkyl, C₁₋₄ alkoxy, and C₁₋₄ haloalkoxy;
R¹³ is C₁₋₆ alkyl, C₃₋₆ cycloalkyl, (C₃₋₄ cycloalkyl)-C₁₋₄ alkyl-, phenyl, or phenyl-C₁₋₄ alkyl-, each of which is optionally substituted with 1, 2, 3, 4, or 5 substituents each independently selected from halogen, —OH, —CN, C₁₋₄ alkyl, C₁₋₄ haloalkyl, C₁₋₄ alkoxy, C₁₋₄ haloalkoxy, C₃₋₄ cycloalkyl, or (C₃₋₄ cycloalkyl)-C₁₋₄ alkyl-;
L¹ is C(R^{L})₂;
each R^{L} is independently H, C₁₋₂ alkyl, C₁₋₂ haloalkyl, C₁₋₂ alkoxy, or C₁₋₂ haloalkoxy;
or two R^{L} together with the carbon atom to which they are attached, optionally form C₃₋₆ cycloalkyl or a 3- to 6-membered heterocycloalkyl, each of which is optionally substituted with 1, 2, 3, or 4 substituents each independently selected from halogen, —OH, C₁₋₄ alkyl, C₁₋₄ haloalkyl, C₁₋₄ alkoxy, and C₁₋₄ haloalkoxy;
t1 is 0 or 1;
t2 is 0, 1, 2, 3, or 4;
t3 is 1 or 2; and
t4 is 0, 1, 2, 3, or 4.

The present invention also provides a pharmaceutical composition containing the compound of Formula I or a pharmaceutically acceptable salt of the compound and a pharmaceutically acceptable excipient or carrier.

The present invention also provides a method for treating or preventing a GIPR-related condition, disease, or disorder in a patient (e.g., a mammal or a human), which method includes administering to the patient (e.g., the mammal or human) the compound of Formula I or a pharmaceutically acceptable salt of the compound.

The present invention also provides the compound of Formula I or a pharmaceutically acceptable salt of the compound for use in treating or preventing a GIPR-related condition, disease, or disorder.

The GIPR-related condition, disease, or disorder includes one selected from diabetes [e.g. Type 1 diabetes mellitus (T1D), Type 2 diabetes mellitus (T2DM), including pre-diabetes], idiopathic T1D (Type 1b), latent autoimmune diabetes in adults (LADA), early-onset T2DM (EOD), youth-onset atypical diabetes (YOAD), maturity onset diabetes of the young (MODY), malnutrition-related diabetes, gestational diabetes, hyperglycemia, insulin resistance, hepatic insulin resistance, impaired glucose tolerance, diabetic neuropathy, diabetic nephropathy, kidney disease [e.g., acute kidney disorder, tubular dysfunction, proinflammatory changes to the proximal tubules, or chronic kidney disease (CKD)], diabetic retinopathy, adipocyte dysfunction, visceral adipose deposition, sleep apnea [e.g. obstructive sleep apnea (OSA)], obesity (including hypothalamic obesity and monogenic obesity) and related comorbidities (e.g., osteoarthritis and urine incontinence), eating disorders (including binge eating syndrome, bulimia nervosa, and syndromic obesity such as Prader-Willi and Bardet-Biedl syndromes), weight gain such as weight gain caused by use of other agents (e.g., caused by use of steroids and/or antipsychotics, or caused by treatment of depression, or caused by use of agents on cognitive function), excessive sugar craving, dyslipidemia [including hyperlipidemia, hypertriglyceridemia, increased total cholesterol, high LDL (low-density lipoprotein) cholesterol, and low HDL (high-density lipoprotein) cholesterol], hyperinsulinemia, nonalcoholic fatty liver disease [NAFLD, including related diseases such as steatosis, nonalcoholic steatohepatitis (NASH), fibrosis, cirrhosis, and hepatocellular carcinoma], cardiovascular disease, atherosclerosis (including coronary artery disease), peripheral vascular disease, hypertension, endothelial dysfunction, impaired vascular compliance, heart failure [e.g. congestive heart failure, heart failure with preserved ejection fraction (HFpEF), heart failure with reduced ejection fraction (HFrEF)], myocardial infarction (e.g. necrosis and apoptosis), stroke, hemorrhagic stroke, ischemic stroke, traumatic brain injury, pulmonary hypertension, restenosis after angioplasty, intermittent claudication, post-prandial lipemia, metabolic acidosis, ketosis, arthritis, osteoporosis, osteoarthritis, Parkinson's disease, left ventricular hypertrophy, peripheral arterial disease, macular degeneration, cataract, glomerulosclerosis, chronic renal failure, metabolic syndrome, syndrome X, premenstrual syndrome, angina pectoris, thrombosis, atherosclerosis, transient ischemic attacks, vascular restenosis, impaired glucose metabolism, conditions of impaired fasting plasma glucose, hyperuricemia, gout, erectile dysfunction, skin and connective tissue disorders, psoriasis, foot ulcerations, ulcerative colitis, hyper apo B lipoproteinemia, Alzheimer's Disease, schizophrenia, impaired cognition, inflammatory bowel disease, short bowel syndrome, Crohn's disease, colitis, irritable bowel syndrome, polycystic ovary syndrome (PCOS), and addiction (e.g., addition to alcohol, nicotine, and/or drug).

The present invention also provides a method for antagonizing a glucose-dependent insulinotropic polypeptide receptor (GIPR), which method includes contacting the GIPR with the compound of Formula I or a pharmaceutically acceptable salt of the compound.

It is to be understood that both the foregoing general description and the following detailed description are exemplary and explanatory only and are not restrictive of the invention, as claimed.

### DETAILED DESCRIPTION OF THE INVENTION

The present invention may be understood more readily by reference to the following detailed description of exemplary embodiments of the invention and the examples included therein.

Some additional exemplary embodiments of the invention are described herein below.

Embodiment A2 is a further embodiment of Embodiment A1, wherein the compound is a compound of Formula Ia:

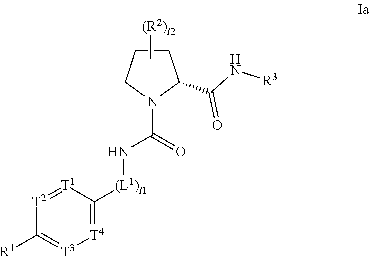

or a pharmaceutically acceptable salt thereof.

Embodiment A3 is a further embodiment of Embodiment A1, wherein the compound is a compound of Formula II:

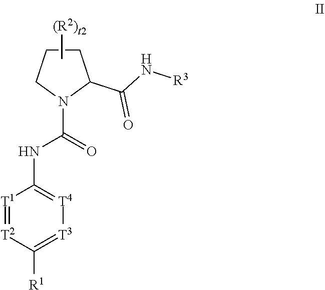

or a pharmaceutically acceptable salt thereof.

Embodiment A4 is a further embodiment of Embodiment A1, wherein the compound is a compound of Formula IIa:

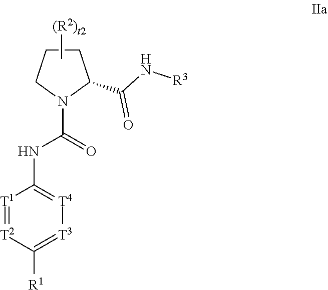

or a pharmaceutically acceptable salt thereof.

Embodiment A5 is a further embodiment of Embodiment A1, wherein the compound is a compound of Formula III:

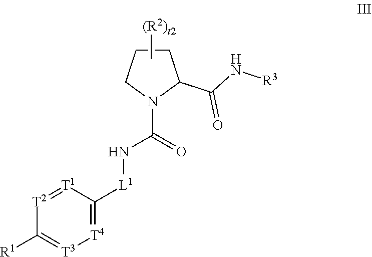

or a pharmaceutically acceptable salt thereof. Embodiment A5-a is a further embodiment of Embodiment A5, wherein L¹ is CH₂. In herein below, when Embodiment A5 is referred to, it includes Embodiment A5 and any of its further embodiments (Embodiment A5-a).

Embodiment A6 is a further embodiment of Embodiment A1, wherein the compound is a compound of Formula IIIa:

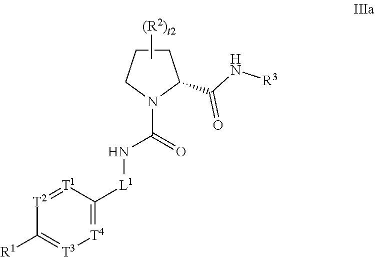

or a pharmaceutically acceptable salt thereof. Embodiment A6-a is a further embodiment of Embodiment A6, wherein L¹ is CH₂. In herein below, when Embodiment A6 is referred to, it includes Embodiment A6 and any of its further embodiments (Embodiment A6-a).

Embodiment A7 is a further embodiment of Embodiment A1, wherein the compound is a compound of Formula IV:

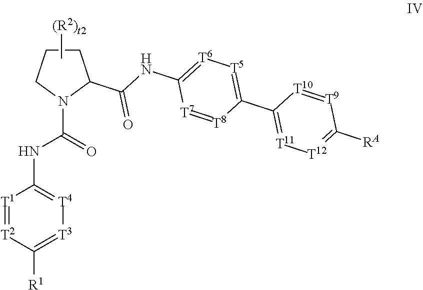

or a pharmaceutically acceptable salt thereof.

Embodiment A7-a is a further embodiment of Embodiment A7, wherein the compound is a compound of Formula IV-1:

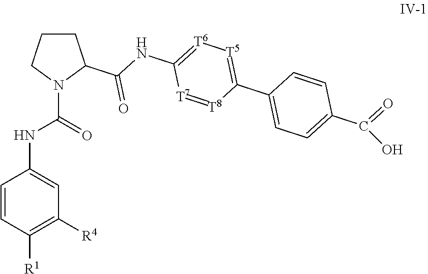

or a pharmaceutically acceptable salt thereof;
R¹ is propan-2-yl, prop-1-en-2-yl, trifluoromethyl, or cyclopropyl;
R⁴ is H, halo, or C₁₋₂ alkyl;
each of T⁵, T⁶, T⁷, and T⁸ is independently CR⁵, or one of T⁵, T⁶, T⁷, and T⁸ is N, and each of the other three of T⁵, T⁶, T⁷, and T⁸ is independently CR⁵; and
each R⁵ is independently H, halogen, —CN, C₁₋₂ alkyl, C₁₋₂ haloalkyl, C₁₋₂ alkoxy, or C₁₋₂ haloalkoxy.

Embodiment A7-a-1 is a further embodiment of Embodiment A7-a, wherein R¹ is propan-2-yl, or trifluoromethyl; R⁴ is H, F, Cl, or C₁₋₂ alkyl; each of T⁵, T⁶, T⁷, and T⁸ is independently CR⁵; and each R⁵ is independently H, F, Cl, C₁₋₂ alkyl, C₁₋₂ haloalkyl, C₁₋₂ alkoxy, or C₁₋₂ haloalkoxy. Embodiment A7-a-2 is a further embodiment of Embodiment A7-a, wherein R¹ is propan-2-yl, or trifluoromethyl; R⁴ is H, F, or C₁₋₂ alkyl (e.g. methyl); each of T⁵, T⁶, T⁷, and T⁸ is independently CR⁵; and each R⁵ is independently H, F, C₁₋₂ alkyl, C₁₋₂ haloalkyl, C₁₋₂ alkoxy, or C₁₋₂ haloalkoxy. Embodiment A7-a-3 is a further embodiment of Embodiment A7-a, wherein R¹ is propan-2-yl, or trifluoromethyl; R⁴ is H, F, or C₁₋₂ alkyl (e.g. methyl); each of T⁵, T⁶, T⁷, and T⁸ is independently CR⁵; and each R⁵ is H. Embodiment A7-a-4 is a further embodiment of Embodiment A7-a, wherein R¹ is propan-2-yl, or trifluoromethyl; R⁴ is H, F, Cl, or C₁₋₂ alkyl; one of T⁵, T⁶, T⁷, and T⁸ is N, and each of the other three of T⁵, T⁶, T⁷, and T⁸ is independently CR⁵; and each R⁵ is independently H, F, Cl, C₁₋₂ alkyl, C₁₋₂ haloalkyl, C₁₋₂ alkoxy, or C₁₋₂ haloalkoxy.

Embodiment A7-b is a further embodiment of Embodiment A7, wherein the compound is a compound of Formula IV-2:

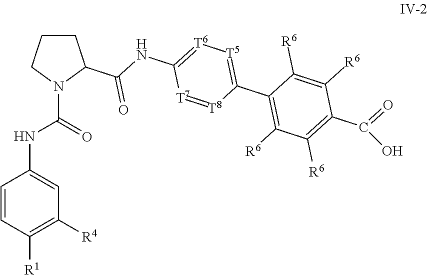

or a pharmaceutically acceptable salt thereof;
R¹ is propan-2-yl, prop-1-en-2-yl, trifluromethyl, or cyclopropyl;
R⁴ is H, halo, or C₁₋₂ alkyl;
each of T⁵, T⁶, T⁷, and T⁸ is independently CR⁵, or one of T⁵, T⁶, T⁷, and T⁸ is N, and each of the other three of T⁵, T⁶, T⁷, and T⁸ is independently CR⁵;
each R⁵ is independently H, halogen, —CN, C₁₋₂ alkyl, C₁₋₂ haloalkyl, C₁₋₂ alkoxy, or C₁₋₂ haloalkoxy; and
each R⁶ is independently H, halogen, —CN, C₁₋₂ alkyl, C₁₋₂ haloalkyl, C₁₋₂ alkoxy, or C₁₋₂ haloalkoxy, provided that at least one of the four R⁶ is other than H (e.g. one or two R⁶ are other than H).

Embodiment A7-b-1 is a further embodiment of Embodiment A7-b, wherein R¹ is propan-2-yl or trifluromethyl; R⁴ is H, F, Cl, or C₁₋₂ alkyl; each of T⁵, T⁶, T⁷, and T⁸ is independently CR⁵; and each R⁵ is independently H, F, Cl, C₁₋₂ alkyl, C₁₋₂ haloalkyl, C₁₋₂ alkoxy, or C₁₋₂ haloalkoxy. Embodiment A7-b-2 is a further embodiment of Embodiment A7-b, wherein R¹ is propan-2-yl, or trifluromethyl; R⁴ is H, F, or C₁₋₂ alkyl (e.g. methyl); each of T⁵, T⁶, T⁷, and T⁸ is independently CR⁵; each R⁵ is independently H, F, C₁₋₂ alkyl, C₁₋₂ haloalkyl, C₁₋₂ alkoxy, or C₁₋₂ haloalkoxy; and each R⁶ is independently H, halogen, C₁₋₂ alkyl, C₁₋₂ haloalkyl, C₁₋₂ alkoxy, or C₁₋₂ haloalkoxy, provided that at least one of the four R⁶ is other than H. Embodiment A7-b-3 is a further embodiment of Embodiment A7-b, wherein R¹ is propan-2-yl, or trifluromethyl; R⁴ is H, F, or C₁₋₂ alkyl (e.g. methyl); each of T⁵, T⁶, T⁷, and T⁸ is independently CR⁵; each R⁵ is H; and each R⁶ is independently H, halogen, C₁₋₂ alkyl, C₁₋₂ haloalkyl, C₁₋₂ alkoxy, or C₁₋₂ haloalkoxy, provided that at least one of the four R⁶ is other than H. Embodiment A7-b-4 is a further embodiment of Embodiment A7-b, wherein R¹ is propan-2-yl or trifluromethyl; R⁴ is H, F, Cl, or C₁₋₂ alkyl; one of T⁵, T⁶, T⁷, and T⁸ is N, and each of the other three of T⁵, T⁶, T⁷, and T⁸ is independently CR⁵; each R⁵ is independently H, F, Cl, C₁₋₂ alkyl, C₁₋₂ haloalkyl, C₁₋₂ alkoxy, or C₁₋₂ haloalkoxy; and each R⁶ is independently H, halogen, C₁₋₂ alkyl, C₁₋₂ haloalkyl, C₁₋₂ alkoxy, or C₁₋₂ haloalkoxy, provided that at least one of the four R⁶ is other than H. Embodiment A7-b-5 is a further embodiment of Embodiment A7-b, wherein R¹ is propan-2-yl or trifluromethyl; R⁴ is H, F, Cl, or C₁₋₂ alkyl; one of T⁵, T⁶, T⁷, and T⁸ is N, and each of the other three of T⁵, T⁶, T⁷, and T⁸ is independently CR⁵; each R⁵ is independently H, F, Cl, C₁₋₂ alkyl, C₁₋₂ haloalkyl, C₁₋₂ alkoxy, or C₁₋₂ haloalkoxy; and each R⁶ is independently H, halogen, C₁₋₂ alkyl, C₁₋₂ haloalkyl, C₁₋₂ alkoxy, or C₁₋₂ haloalkoxy, provided that one of the four R⁶ is other than H and that the other three of the four R⁶ are H.

In herein below, when Embodiment A7 is referred to, it includes Embodiment A7 and any of its further embodiments (Embodiment A7-a, A7-b, Embodiments A7-a-1 to A7-a-4, and A7-b-1 to A7-b-5).

Embodiment A8 is a further embodiment of Embodiment A1, wherein the compound is a compound of Formula IVa:

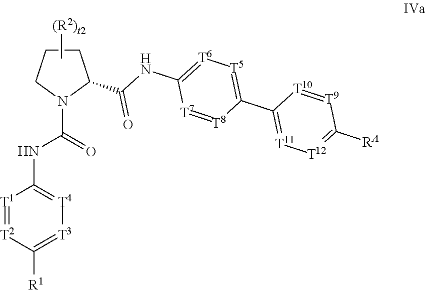

or a pharmaceutically acceptable salt thereof.

Embodiment A8-a is a further embodiment of Embodiment A8, wherein the compound is a compound of Formula IVa-1:

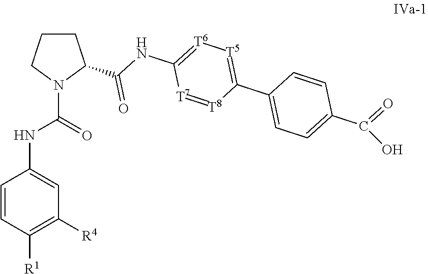

or a pharmaceutically acceptable salt thereof;
R¹ is propan-2-yl, prop-1-en-2-yl, trifluromethyl, or cyclopropyl;
R⁴ is H, halo, or C₁₋₂ alkyl;
each of T⁵, T⁶, T⁷, and T⁸ is independently CR⁵, or one of T⁵, T⁶, T⁷, and T⁸ is N, and each of the other three of T⁵, T⁶, T⁷, and T⁸ is independently CR⁵; and
each R⁵ is independently H, halogen, —CN, C₁₋₂ alkyl, C₁₋₂ haloalkyl, C₁₋₂ alkoxy, or C₁₋₂ haloalkoxy.

Embodiment A8-a-1 is a further embodiment of Embodiment A8-a, wherein R¹ is propan-2-yl or trifluromethyl; R⁴ is H, F, Cl, or C₁₋₂ alkyl; each of T⁵, T⁶, T⁷, and T⁸ is independently CR⁵; and each R⁵ is independently H, F, Cl, C₁₋₂ alkyl, C₁₋₂ haloalkyl, C₁₋₂ alkoxy, or C₁₋₂ haloalkoxy. Embodiment A8-a-2 is a further embodiment of Embodiment A8-a, wherein R¹ is propan-2-yl, or trifluromethyl; R⁴ is H, F, or C₁₋₂ alkyl (e.g. methyl); each of T⁵, T⁶, T⁷, and T⁸ is independently CR⁵; and each R⁵ is independently H, F, C₁₋₂ alkyl, C₁₋₂ haloalkyl, C₁₋₂ alkoxy, or C₁₋₂ haloalkoxy. Embodiment A8-a-3 is a further embodiment of Embodiment A8-a, wherein R¹ is propan-2-yl, or trifluromethyl; R⁴ is H, F, or C₁₋₂ alkyl (e.g. methyl); each of T⁵, T⁶, T⁷, and T⁸ is independently CR⁵; and each R⁵ is H. Embodiment A8-a-4 is a further embodiment of Embodiment A8-a, wherein R¹ is propan-2-yl, or trifluromethyl; R⁴ is H, F, Cl, or C₁₋₂ alkyl; one of T⁵, T⁶, T⁷, and T⁸ is N, and each of the other three of T⁵, T⁶, T⁷, and T⁸ is independently CR⁵; and each R⁵ is independently H, F, Cl, C₁₋₂ alkyl, C₁₋₂ haloalkyl, C₁₋₂ alkoxy, or C₁₋₂ haloalkoxy.

Embodiment A8-b is a further embodiment of Embodiment A8, wherein the compound is a compound of Formula IVa-2:

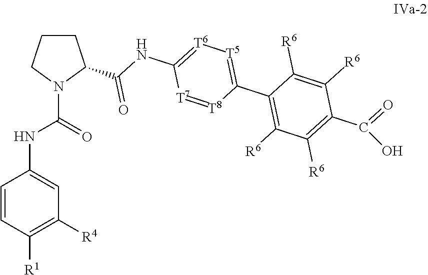

or a pharmaceutically acceptable salt thereof;
R¹ is propan-2-yl, prop-1-en-2-yl, trifluromethyl, or cyclopropyl;
R⁴ is H, halo, or C₁₋₂ alkyl;
each of T⁵, T⁶, T⁷, and T⁸ is independently CR⁵, or one of T⁵, T⁶, T⁷, and T⁸ is N, and each of the other three of T⁵, T⁶, T⁷, and T⁸ is independently CR⁵;
each R⁵ is independently H, halogen, —CN, C₁₋₂ alkyl, C₁₋₂ haloalkyl, C₁₋₂ alkoxy, or C₁₋₂ haloalkoxy; and
each R⁶ is independently H, halogen, —CN, C₁₋₂ alkyl, C₁₋₂ haloalkyl, C₁₋₂ alkoxy, or C₁₋₂ haloalkoxy, provided that at least one of the four R⁶ is other than H (e.g. one or two R⁶ are other than H).

Embodiment A8-b-1 is a further embodiment of Embodiment A8-b, wherein R¹ is propan-2-yl or trifluromethyl; R⁴ is H, F, Cl, or C₁₋₂ alkyl; each of T⁵, T⁶, T⁷, and T⁸ is independently CR⁵; and each R⁵ is independently H, F, Cl, C₁₋₂ alkyl, C₁₋₂ haloalkyl, C₁₋₂ alkoxy, or C₁₋₂ haloalkoxy. Embodiment A8-b-2 is a further embodiment of Embodiment A8-b, wherein R¹ is propan-2-yl or trifluromethyl; R⁴ is H, F, or C₁₋₂ alkyl (e.g. methyl); each of T⁵, T⁶, T⁷, and T⁸ is independently CR⁵; each R⁵ is independently H, F, C₁₋₂ alkyl, C₁₋₂ haloalkyl, C₁₋₂ alkoxy, or C₁₋₂ haloalkoxy; and each R⁶ is independently H, halogen, C₁₋₂ alkyl, C₁₋₂ haloalkyl, C₁₋₂ alkoxy, or C₁₋₂ haloalkoxy, provided that at least one of the four R⁶ is other than H. Embodiment A8-b-3 is a further embodiment of Embodiment A8-b, wherein R¹ is propan-2-yl or trifluromethyl; R⁴ is H, F, or C₁₋₂ alkyl (e.g. methyl); each of T⁵, T⁶, T⁷, and T⁸ is independently CR⁵; each R⁵ is H; and each R⁶ is independently H, halogen, C₁₋₂ alkyl, C₁₋₂ haloalkyl, C₁₋₂ alkoxy, or C₁₋₂ haloalkoxy, provided that at least one of the four R⁶ is other than H. Embodiment A8-b-4 is a further embodiment of Embodiment A8-b, wherein R¹ is propan-2-yl or trifluromethyl; R⁴ is H, F, Cl, or C₁₋₂ alkyl; one of T⁵, T⁶, T⁷, and T⁸ is N, and each of the other three of T⁵, T⁶, T⁷, and T⁸ is independently CR⁵; each R⁵ is independently H, F, Cl, C₁₋₂ alkyl, C₁₋₂ haloalkyl, C₁₋₂ alkoxy, or C₁₋₂ haloalkoxy; and each R⁶ is independently H, halogen, C₁₋₂ alkyl, C₁₋₂ haloalkyl, C₁₋₂ alkoxy, or C₁₋₂ haloalkoxy, provided that at least one of the four R⁶ is other than H. Embodiment A8-b-5 is a further embodiment of Embodiment A8-b, wherein R¹ is propan-2-yl or trifluromethyl; R⁴ is H, F, Cl, or C₁₋₂ alkyl; one of T⁵, T⁶, T⁷, and T⁸ is N, and each of the other three of T⁵, T⁶, T⁷, and T⁸ is independently CR⁵; each R⁵ is independently H, F, Cl, C₁₋₂ alkyl, C₁₋₂ haloalkyl, C₁₋₂ alkoxy, or C₁₋₂ haloalkoxy; and each R⁶ is independently H, halogen, C₁₋₂ alkyl, C₁₋₂ haloalkyl, C₁₋₂ alkoxy, or C₁₋₂ haloalkoxy, provided that one of the four R⁶ is other than H and that the other three R⁶ are H.

In herein below, when Embodiment A8 is referred to, it includes Embodiment A8 and any of its further embodiments (Embodiment A8-a, Embodiment A8-b, Embodiments A8-a-1 to A8-a-4, and A8-b-1 to A8-b-5).

Embodiment A9 is a further embodiment of Embodiment A1, wherein the compound is a compound of Formula V:

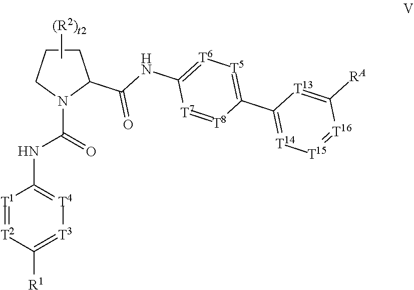

or a pharmaceutically acceptable salt thereof.

Embodiment A10 is a further embodiment of Embodiment A1, wherein the compound is a compound of Formula Va:

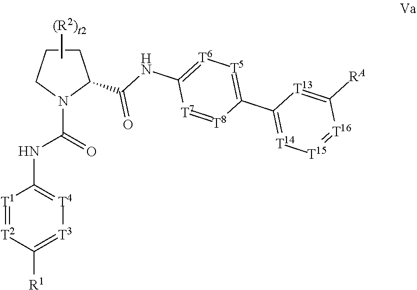

or a pharmaceutically acceptable salt thereof.

Embodiment A11 is a further embodiment of Embodiment A1, wherein the compound is a compound of Formula VI:

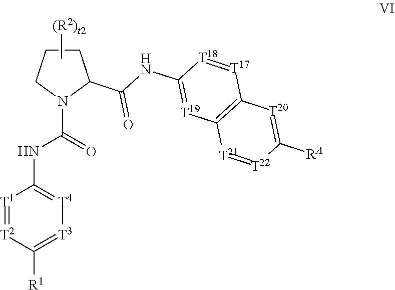

or a pharmaceutically acceptable salt thereof.

Embodiment A12 is a further embodiment of Embodiment A1, wherein the compound is a compound of Formula VIa:

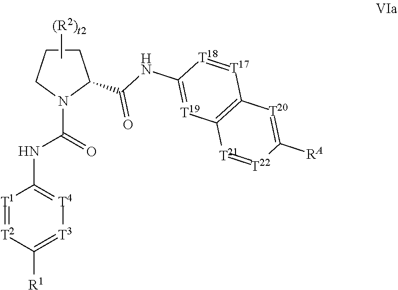

or a pharmaceutically acceptable salt thereof.

Embodiment A13 is a further embodiment of Embodiment A1, wherein the compound is a compound of Formula VII:

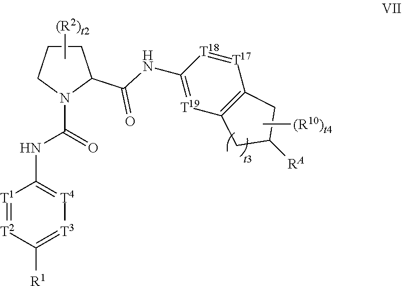

or a pharmaceutically acceptable salt thereof.

Embodiment A14 is a further embodiment of Embodiment A1, wherein the compound is a compound of Formula VIIa:

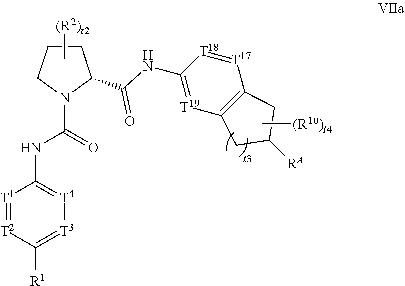

or a pharmaceutically acceptable salt thereof.

Embodiment A15 is a further embodiment of any one of Embodiments A1 to A14, including the further embodiments described herein, wherein R¹ is halogen, —CN, C₁₋₈ alkyl, C₂₋₈ alkenyl, (C₃₋₆ cycloalkyl)-C₁₋₄ alkyl-, or C₃₋₆ cycloalkyl, wherein each of the C₁₋₈ alkyl, C₂₋₈ alkenyl, (C₃₋₆ cycloalkyl)-C₁₋₄ alkyl-, or C₃₋₆ cycloalkyl is optionally substituted with 1, 2, 3, 4, 5, or 6 substituents each independently selected from halogen, —OH, —CN, C₁₋₄ alkyl, C₁₋₄ haloalkyl, C₁₋₄ alkoxy, and C₁₋₄ haloalkoxy. Embodiment A15-a is a further embodiment of Embodiment A15, wherein R¹ is C₁₋₈ alkyl optionally substituted with 1, 2, 3, 4, 5, or 6 substituents each independently selected from halogen, —OH, —CN, C₁₋₄ alkyl, C₁₋₄ haloalkyl, C₁₋₄ alkoxy, and C₁₋₄ haloalkoxy. Embodiment A15-b is a further embodiment of Embodiment A15, wherein R¹ is C₂₋₆ alkyl optionally substituted with 1, 2, 3, 4, 5, or 6 substituents each independently selected from halogen, —OH, —CN, C₁₋₄ alkoxy, and C₁₋₄ haloalkoxy. Embodiment A15-c is a further embodiment of Embodiment A15, wherein R¹ is C₂₋₄ alkyl optionally substituted with 1, 2, 3, 4, 5, or 6 substituents each independently selected from halogen, —OH, —CN, C₁₋₄ alkoxy, and C₁₋₄ haloalkoxy. Embodiment A15-d is a further embodiment of Embodiment A15, wherein R¹ is C₂₋₄ alkyl optionally substituted with 1, 2, 3, or 4 substituents each independently selected from halogen, —OH, —CN, C₁₋₄ alkoxy, and C₁₋₄ haloalkoxy. Embodiment A15-e is a further embodiment of Embodiment A15, wherein R¹ is C₂₋₄ alkyl optionally substituted with 1, 2, 3, or 4 substituents each independently selected from halogen, —OH, C₁₋₄ alkoxy, and C₁₋₄ haloalkoxy. In herein below, when Embodiment A15 is referred to, it includes Embodiment A16 and any of its further embodiments (Embodiments A15-a to A15-e).

Embodiment A16 is a further embodiment of any one of Embodiments A1 to A14, including the further embodiments described herein, wherein R¹ is halogen, C₃₋₆ alkyl, C₃₋₆ alkenyl, (C₃₋₆ cycloalkyl)-C₁₋₄ alkyl-, or C₃₋₆ cycloalkyl, wherein each of the C₃₋₆ alkyl, C₃₋₆ alkenyl, (C₃₋₆ cycloalkyl)-C₁₋₄ alkyl-, or C₃₋₆ cycloalkyl is optionally substituted with 1, 2, 3, 4, 5, or 6 substituents each independently selected from halogen, —OH, —CN, C₁₋₄ alkyl, C₁₋₄ haloalkyl, C₁₋₄ alkoxy, and C₁₋₄ haloalkoxy. Embodiment A16-a is a further embodiment of Embodiment A16, wherein R¹ is cyclobutyl optionally substituted with 1, 2, 3, 4, 5, or 6 substituents each independently selected from halogen, —OH, —CN, C₁₋₄ alkyl, C₁₋₄ haloalkyl, C₁₋₄ alkoxy, and C₁₋₄ haloalkoxy. Embodiment A16-b is a further embodiment of Embodiment A16, wherein R¹ is cyclobutyl optionally substituted with 1 or 2 substituents each independently selected from halogen, —OH, —CN, C₁₋₄ alkyl, C₁₋₄ haloalkyl, C₁₋₄ alkoxy, and C₁₋₄ haloalkoxy. Embodiment A16-c is a further embodiment of Embodiment A16, wherein R¹ is C₃₋₄ alkyl optionally substituted with 1, 2, 3, 4, 5, or 6 substituents each independently selected from halogen, —OH, —CN, C₁₋₄ alkoxy, and C₁₋₄ haloalkoxy. Embodiment A16-d is a further embodiment of Embodiment A16, wherein R¹ is C₃₋₄ alkyl optionally substituted with 1, 2, 3, or 4 substituents each independently selected from halogen, —OH, —CN, C₁₋₄ alkoxy, and C₁₋₄ haloalkoxy. Embodiment A16-e is a further embodiment of Embodiment A16, wherein R¹ is C₃₋₄ alkyl optionally substituted with 1, 2, 3, or 4 substituents each independently selected from halogen, —OH, C₁₋₄ alkoxy, and C₁₋₄ haloalkoxy. In herein below, when Embodiment A16 is referred to, it includes Embodiment A16 and any of its further embodiments (Embodiments A16-a to A16-e).

Embodiment A17 is a further embodiment of any one of Embodiments A1 to A14, including the further embodiments described herein, wherein R¹ is cyclopropyl, cyclobutyl, R^{1a}, R^{1b}, or R^{1c},

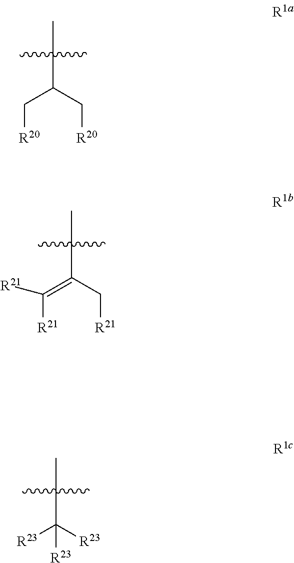

wherein each of the cyclopropyl or cyclobutyl is optionally substituted with 1, 2, 3, or 4 R^{S};
each R²⁰ is independently H, halogen, —OH, C₁₋₂ alkyl, C₁₋₂ haloalkyl, C₁₋₂ alkoxy, or C₁₋₂ haloalkoxy;
each R²¹ is independently H, C₁₋₂ alkyl, or C₁₋₂ haloalkyl;
R²² is H, halogen, C₁₋₂ alkyl, C₁₋₂ hydroxylalkyl, C₁₋₂ haloalkyl, C₁₋₂ alkoxy, or C₁₋₂ haloalkoxy;
each R²³ is independently halogen, C₁₋₂ alkyl, C₁₋₂ hydroxylalkyl, C₁₋₂ haloalkyl, C₁₋₂ alkoxy, or C₁₋₂ haloalkoxy; and
each R^{S} is independently halogen, —OH, C₁₋₂ alkyl, C₁₋₂ hydroxylalkyl, C₁₋₂ haloalkyl, C₁₋₂ alkoxy, or C₁₋₂ haloalkoxy. Embodiment A17-a is a further embodiment of Embodiment A17, wherein R¹ is R^{1a}. Embodiment A17-b is a further embodiment of Embodiment A17, wherein R¹ is R^{1a} and each R²⁰ is independently H, halogen, —OH, C₁₋₂ alkyl, or C₁₋₂ haloalkyl. Embodiment A17-b is a further embodiment of Embodiment A17, wherein R¹ is R^{1a} and each R²⁰ is independently H, —OH, C₁₋₂ alkyl, or C₁₋₂ haloalkyl. Embodiment A17-c is a further embodiment of Embodiment A17, wherein R¹ is R^{1a} and each R²⁰ is independently H, —OH, or C₁₋₂ alkyl. Embodiment A17-d is a further embodiment of Embodiment A17, wherein R¹ is R^{1b}; each R²¹ is independently H or C₁₋₂ alkyl; and R²² is H, C₁₋₂ alkyl, or C₁₋₂ hydroxylalkyl. Embodiment A17-d is a further embodiment of Embodiment A17, wherein R¹ is R^{1b}; each R²¹ is independently H or C₁₋₂ alkyl; and R²² is H or C₁₋₂ alkyl. Embodiment A17-e is a further embodiment of Embodiment A17, wherein R¹ is R^{1c}. Embodiment A17-f is a further embodiment of Embodiment A17, wherein R¹ is R^{1c}; and each R²³ is independently halogen, C₁₋₂ alkyl, C₁₋₂ hydroxylalkyl, or C₁₋₂ haloalkyl. Embodiment A17-g is a further embodiment of Embodiment A17, wherein R¹ is R^{1c}; and each R²³ is independently C₁₋₂ alkyl, C₁₋₂ hydroxylalkyl, or C₁₋₂ haloalkyl. Embodiment A17-h is a further embodiment of Embodiment A17, wherein R¹ is R^{1c}; and each R²³ is independently C₁₋₂ alkyl. Embodiment A17-i is a further embodiment of Embodiment A17, wherein R¹ is R^{1c}; and each R²³ is methyl. In herein below, when Embodiment A17 is referred to, it includes Embodiment A18 and any of its further embodiments (Embodiments A17-a to A17-i).

Embodiment A18 is a further embodiment of any one of Embodiments A1 to A14, including the further embodiments described herein, wherein R¹ is propan-2-yl, prop-1-en-2-yl, trifluoromethyl, or cyclopropyl. Embodiment A18-a is a further embodiment of Embodiment A18, wherein R¹ is propan-2-yl, prop-1-en-2-yl, or cyclopropyl. Embodiment A18-b is a further embodiment of Embodiment A18, wherein R¹ is propan-2-yl, prop-1-en-2-yl, or trifluoromethyl. Embodiment A18-c is a further embodiment of Embodiment A18, wherein R¹ is trifluoromethyl. In herein below, when Embodiment A18 is referred to, it includes Embodiment A18 and any of its further embodiments (Embodiments A18-a to A18-c).

Embodiment A19 is a further embodiment of any one of Embodiments A1 to A14, including the further embodiments described herein, wherein R¹ is propan-2-yl.

Embodiment A20 is a further embodiment of any one of Embodiments A1 to A14, including the further embodiments described herein, wherein R¹ is prop-1-en-2-yl.

Embodiment A21 is a further embodiment of any one of Embodiments A1 to A14, including the further embodiments described herein, wherein R¹ is cyclopropyl or cyclobutyl, each optionally substituted with 1 or 2 substituents, each of which is independently C₁₋₂ alkyl or C₁₋₂ haloalkyl.

Embodiment A21-a is a further embodiment of Embodiment A21, wherein R¹ is cyclopropyl or cyclobutyl, each optionally substituted with one C₁₋₂ alkyl or C₁₋₂ haloalkyl (e.g. CF₃). Embodiment A21-b is a further embodiment of Embodiment A21, wherein R¹ is cyclopropyl optionally substituted with one C₁₋₂ alkyl or C₁₋₂ haloalkyl (e.g. CF₃). Embodiment A21-c is a further embodiment of Embodiment A21, wherein R¹ is cyclopropyl. Embodiment A21-d is a further embodiment of Embodiment A21, wherein R¹ is cyclobutyl. In herein below, when Embodiment A21 is referred to, it includes Embodiment A21 and any of its further embodiments (Embodiments A21-a to A21-d).

Embodiment A22 is a further embodiment of any one of Embodiments A1 to A14, including the further embodiments described herein, wherein R¹ is C₁₋₄ haloalkyl or halo. Embodiment A22-a is a further embodiment of Embodiment A22, wherein R¹ is C₁₋₄ haloalkyl, for example, R¹ is C₁₋₂ haloalkyl. Embodiment A22-b is a further embodiment of Embodiment A22, wherein R¹ is C₁₋₂ haloalkyl, for example, R¹ is C₁₋₂ fluoroalkyl. Embodiment A22-c is a further embodiment of Embodiment A22, wherein R¹ is trifluoromethyl. Embodiment A22-d is a further embodiment of Embodiment A22, wherein R¹ is halo, for example, Cl. In herein below, when Embodiment A22 is referred to, it includes Embodiment A22 and any of its further embodiments (Embodiments A22-a and A22-d).

Embodiment A23 is a further embodiment of any one of Embodiments A1 to A22, including the further embodiments described herein, wherein each of T¹, T², T³, and T⁴ is independently CR⁴.

Embodiment A23-a is a further embodiment of Embodiment A23, wherein each of T¹, T², and T⁴ is CH; and T³ is CR⁴. Embodiment A23-b is a further embodiment of Embodiment A23, wherein each of T¹, T², and T⁴ is CH; T³ is CR⁴; and R⁴ is H, halo, C₁₋₂ alkyl, or C₁₋₂ haloalkyl. Embodiment A23-c is a further embodiment of Embodiment A23, wherein each of T¹, T², and T⁴ is CH; T³ is CR⁴; and R⁴ is H, F, or methyl. In herein below, when Embodiment A23 is referred to, it includes Embodiment A23 and any one of its further embodiments (Embodiments A23-a to A23-c).

Embodiment A24 is a further embodiment of any one of Embodiments A1 to A22, including the further embodiments described herein, wherein one of T¹, T², T³, and T⁴ is N, and the other three are each independently CR⁴. Embodiment A24-a is a further embodiment of Embodiment A24, wherein T¹ is N, and each of T², T³, and T⁴ is independently CR⁴. In herein below, when Embodiment A24 is referred to, it includes Embodiment A24 and any one of its further embodiments (Embodiment A24-a).

Embodiment A25 is a further embodiment of any one of Embodiments A1 to A22, including the further embodiments described herein, wherein T² is N, and each of T¹, T³, and T⁴ is independently CR⁴.

Embodiment A26 is a further embodiment of any one of Embodiments A1 to A22, including the further embodiments described herein, wherein two of T¹, T², T³, and T⁴ are N, and the other two are each independently CR⁴.

Embodiment A27 is a further embodiment of any one of Embodiments A1 to A26, including the further embodiments described herein, wherein each R⁴ is independently H, halo, C₁₋₂ alkyl, or C₁₋₂ haloalkyl.

Embodiment A28 is a further embodiment of any one of Embodiments A1 to A26, including the further embodiments described herein, wherein each R⁴ is independently H, halo, or C₁₋₂ alkyl. Embodiment A28-a is a further embodiment of Embodiment A28, wherein each R⁴ is independently H, F, or methyl. Embodiment A28-b is a further embodiment of Embodiment A28, wherein each R⁴ is independently H or F. Embodiment A28-c is a further embodiment of Embodiment A28, wherein each R⁴ is independently H or F. In herein below, when Embodiment A28 is referred to, it includes Embodiment A28 and any one of its further embodiments (Embodiments A28-a and A28-b).

Embodiment A29 is a further embodiment of any one of Embodiments A1 to A26, including the further embodiments described herein, wherein each R⁴ is independently H or C₁₋₂ alkyl.

Embodiment A29-a is a further embodiment of Embodiment A29, wherein each R⁴ is independently H or methyl. In herein below, when Embodiment A29 is referred to, it includes Embodiment A29 and any one of its further embodiments (Embodiment A29-a).

Embodiment A30 is a further embodiment of any one of Embodiments A1 to A26, including the further embodiments described herein, wherein each R⁴ is H.

Embodiment A31 is a further embodiment of any one of Embodiments A1 to A30, including the further embodiments described herein, wherein each R² is independently halogen, —OH, C₁₋₄ alkyl, C₁₋₄ hydroxylalkyl, C₁₋₄ haloalkyl, C₁₋₄ alkoxy, C₁₋₄ haloalkoxy, C₃₋₄ cycloalkyl, or (C₃₋₄ cycloalkyl)-C₁₋₄ alkyl-; and t2 is 0 or 1. Embodiment A31-a is a further embodiment of Embodiment A31, wherein each R² is independently halogen, —OH, C₁₋₂ alkyl, C₁₋₂ hydroxylalkyl, C₁₋₂ haloalkyl, C₁₋₂ alkoxy, C₁₋₂ haloalkoxy, or C₃₋₄ cycloalkyl; and t2 is 0 or 1. Embodiment A31-b is a further embodiment of Embodiment A31, wherein R² is —OH, C₁₋₂ alkyl, C₁₋₂ hydroxylalkyl, C₁₋₂ haloalkyl, C₁₋₂ alkoxy, or C₁₋₂ haloalkoxy; and t2 is 0 or 1. Embodiment A31-c is a further embodiment of Embodiment A31, wherein R² is —OH, C₁₋₂ alkyl, or C₁₋₂ alkoxy; and t2 is 0 or 1. In herein below, when Embodiment A31 is referred to, it includes Embodiment A31 and any one of its further embodiments (Embodiments A31-a to A31-c).

Embodiment A32 is a further embodiment of any one of Embodiments A1 to A30, including the further embodiments described herein, wherein t2 is 0.

Embodiment A33 is a further embodiment of any one of Embodiments A1 to A30, including the further embodiments described herein, wherein t2 is 2 or 3; and two R², which are attached to two adjacent ring carbon atoms of the proline ring in Formula I, together with the two ring carbon atoms to which they are attached, form C₃₋₆ cycloalkyl that is optionally substituted with 1, 2, 3, or 4 substituents each independently selected from halogen, —OH, C₁₋₄ alkyl, C₁₋₄ haloalkyl, C₁₋₄ alkoxy, and C₁₋₄ haloalkoxy. Embodiment A33-a is a further embodiment of Embodiment A33, wherein t2 is 2; and two R², which are attached to two adjacent ring carbon atoms of the proline ring in Formula I, together with the two ring carbon atoms to which they are attached, form C₃₋₆ cycloalkyl that is optionally substituted with 1, 2, 3, or 4 substituents each independently selected from halogen, —OH, C₁₋₄ alkyl, C₁₋₄ haloalkyl, C₁₋₄ alkoxy, and C₁₋₄ haloalkoxy. Embodiment A33-b is a further embodiment of Embodiment A33, wherein t2 is 2; and two R², which are attached to two adjacent ring carbon atoms of the proline ring in Formula I, together with the two ring carbon atoms to which they are attached, form cyclopropyl that is optionally substituted with 1, 2, 3, or 4 substituents each independently selected from halogen, —OH, C₁₋₄ alkyl, C₁₋₄ haloalkyl, C₁₋₄ alkoxy, and C₁₋₄ haloalkoxy. Embodiment A33-c is a further embodiment of Embodiment A33, wherein t2 is 2; and two R², which are attached to two adjacent ring carbon atoms of the proline ring in Formula I, together with the two ring carbon atoms to which they are attached, form cyclopropyl fused to the proline ring, and the resulting fused bicyclic ring is a 3-azabicyclo[3.1.0]hexane ring. In herein below, when Embodiment A33 is referred to, it includes Embodiment A33 and any one of its further embodiments (Embodiments A33-a to A31-c).

Embodiment A34 is a further embodiment of any one of Embodiments A1 to A10, and A15 to A33, including the further embodiments described herein, wherein each of T⁵, T⁶, T⁷, and T⁸ is independently CR⁵. Embodiment A34-a is a further embodiment of Embodiment A34, wherein each of T⁵, T⁶, T⁷, and T⁸ is CH. Embodiment A34-b is a further embodiment of Embodiment A34, wherein three of T⁵, T⁶, T⁷, and T⁸ are CH; one of T⁵, T⁶, T⁷, and T⁸ is CR⁵; and R⁵ is halogen, —CN, C₁₋₂ alkyl, C₁₋₂ haloalkyl, C₁₋₂ alkoxy, or C₁₋₂ haloalkoxy. Embodiment A34-c is a further embodiment of Embodiment A34, wherein three of T⁵, T⁶, T⁷, and T⁸ are CH; one of T⁵, T⁶, T⁷, and T⁸ is CR⁵; and R⁵ is F, Cl, C₁₋₂ alkyl, C₁₋₂ haloalkyl, C₁₋₂ alkoxy, or C₁₋₂ haloalkoxy. Embodiment A34-d is a further embodiment of Embodiment A34, wherein three of T⁵, T⁶, T⁷, and T⁸ are CH; one of T⁵, T⁶, T⁷, and T⁸ is CR⁵; and R⁵ is F, methyl, or methoxy. Embodiment A34-e is a further embodiment of Embodiment A34, wherein at least one of the four R⁵ in T⁵, T⁶, T⁷, and T⁸ is other than H. In herein below, when Embodiment A34 is referred to, it includes Embodiment A34 and any one of its further embodiments (Embodiments A34-a to A34-e).

Embodiment A35 is a further embodiment of any one of Embodiments A1 to A10, and A15 to A33, including the further embodiments described herein, wherein one of T⁵, T⁶, T⁷, and T⁸ is N and the other three are each independently CR⁵. Embodiment A35-a is a further embodiment of Embodiment A35, wherein each of the three R⁵ is H. Embodiment A35-b is a further embodiment of Embodiment A35, wherein one of the three R⁵ is halogen, —CN, C₁₋₂ alkyl, C₁₋₂ haloalkyl, C₁₋₂ alkoxy, or C₁₋₂ haloalkoxy; and the other two R⁵ are H. Embodiment A35-c is a further embodiment of Embodiment A35, wherein one of the three R⁵ is F, Cl, C₁₋₂ alkyl, C₁₋₂ haloalkyl, C₁₋₂ alkoxy, or C₁₋₂ haloalkoxy; and the other two R⁵ are H. Embodiment A35-d is a further embodiment of Embodiment A35, wherein one of the three R⁵ is F, methyl, or methoxy; and the other two R⁵ are H.

In herein below, when Embodiment A35 is referred to, it includes Embodiment A35 and any one of its further embodiments (Embodiments A35-a to A35-d).

Embodiment A36 is a further embodiment of any one of Embodiments A1 to A10, and A15 to A33, including the further embodiments described herein, wherein T⁵ is N and each of T⁶, T⁷, and T³ is independently CR⁵. Embodiment A36-a is a further embodiment of Embodiment A36, wherein each of the three R⁵ is H. Embodiment A36-b is a further embodiment of Embodiment A36, wherein one of the three R⁵ (e.g. the R⁵ in T⁸) is halogen, —CN, C₁₋₂ alkyl, C₁₋₂ haloalkyl, C₁₋₂ alkoxy, or C₁₋₂ haloalkoxy; and the other two R⁵ are H. Embodiment A36-c is a further embodiment of Embodiment A36, wherein one of the three R⁵ (e.g. the R⁵ in T⁸) is F, Cl, C₁₋₂ alkyl, C₁₋₂ haloalkyl, C₁₋₂ alkoxy, or C₁₋₂ haloalkoxy; and the other two R⁵ are H. Embodiment A36-d is a further embodiment of Embodiment A36, wherein one of the three R⁵ (e.g. the R⁵ in T⁸) is F, methyl, or methoxy; and the other two R⁵ are H. In herein below, when Embodiment A36 is referred to, it includes Embodiment A36 and any one of its further embodiments (Embodiments A36-a to A36-d).

Embodiment A37 is a further embodiment of any one of Embodiments A1 to A10, and A15 to A33, including the further embodiments described herein, wherein T⁶ is N and each of T⁵, T⁷, and T³ is independently CR⁵. Embodiment A37-a is a further embodiment of Embodiment A37, wherein each of the three R⁵ is H. Embodiment A37-b is a further embodiment of Embodiment A37, wherein one of the three R⁵ is halogen, —CN, C₁₋₂ alkyl, C₁₋₂ haloalkyl, C₁₋₂ alkoxy, or C₁₋₂ haloalkoxy; and the other two R⁵ are H. Embodiment A37-c is a further embodiment of Embodiment A37, wherein one of the three R⁵ is F, Cl, C₁₋₂ alkyl, C₁₋₂ haloalkyl, C₁₋₂ alkoxy, or C₁₋₂ haloalkoxy; and the other two R⁵ are H. Embodiment A37-d is a further embodiment of Embodiment A37, wherein one of the three R⁵ is F, methyl, or methoxy; and the other two R⁵ are H. In herein below, when Embodiment A37 is referred to, it includes Embodiment A37 and any one of its further embodiments (Embodiments A37-a to A37-d).

Embodiment A38 is a further embodiment of any one of Embodiments A1 to A10, and A15 to A33, including the further embodiments described herein, wherein two of T⁵, T⁶, T⁷, and T⁸ are N and the other two are each independently CR⁵.

Embodiment A39 is a further embodiment of any one of Embodiments A1 to A10, and A15 to A38, wherein each R⁵ is independently H, halo, C₁₋₂ alkyl, or C₁₋₂ haloalkyl.

Embodiment A40 is a further embodiment of any one of Embodiments A1 to A10, and A15 to A38, including the further embodiments described herein, wherein each R⁵ is independently H, halo, or C₁₋₂ alkyl.

Embodiment A41 is a further embodiment of any one of Embodiments A1 to A10, and A15 to A38, including the further embodiments described herein, wherein each R⁵ is independently H or halo.

Embodiment A42 is a further embodiment of any one of Embodiments A1 to A10, and A15 to A38, including the further embodiments described herein, wherein each R⁵ is H.

Embodiment A43 is a further embodiment of any one of Embodiments A1 to A8, and A15 to 35 A42, including the further embodiments described herein, each of T⁹, T¹⁰, T¹¹, and T¹² is independently CR⁶. Embodiment A43-a is a further embodiment of Embodiment A43, wherein each of T⁹, T¹⁰, T¹¹, and T¹² is CH. Embodiment A43-b is a further embodiment of Embodiment A43, wherein three of T⁹, T¹⁰, T¹¹, and T¹² are CH; one of T⁹, T¹⁰, T¹¹, and T¹² (e.g. T⁹) is CR⁶; and R⁶ is halogen, —CN, C₁₋₂ alkyl, C₁₋₂ haloalkyl, C₁₋₂ alkoxy, or C₁₋₂ haloalkoxy. Embodiment A43-c is a further embodiment of Embodiment A43, wherein three of T⁹, T¹⁰, T¹¹, and T¹² are CH; one of T⁹, T¹⁰, T¹¹, and T¹² (e.g. T⁹) is CR⁶; and R⁶ is F, Cl, C₁₋₂ alkyl, C₁₋₂ haloalkyl, C₁₋₂ alkoxy, or C₁₋₂ haloalkoxy. Embodiment A43-d is a further embodiment of Embodiment A43, wherein three of T⁹, T¹⁰, T¹¹, and T¹² are CH; one of T⁹, T¹⁰, T¹¹, and T¹² (e.g. T⁹) is CR⁶; and R⁶ is F, methyl, or methoxy. Embodiment A43-e is a further embodiment of Embodiment A43, wherein at least one of the four R⁶ in T⁹, T¹⁰, T¹¹, and T¹² is other than H. In herein below, when Embodiment A43 is referred to, it includes Embodiment A43 and any one of its further embodiments (Embodiments A43-a to A43-e).

Embodiment A44 is a further embodiment of any one of Embodiments A1 to A8, and A15 to A42, including the further embodiments described herein, wherein one of T⁹, T¹⁰, T¹¹, and T¹² is N and the other three are each independently CR⁶. Embodiment A44-a is a further embodiment of Embodiment A44, wherein each of the three R⁶ is H. Embodiment A44-b is a further embodiment of Embodiment A44, wherein one of the three R⁶ is halogen, —CN, C₁₋₂ alkyl, C₁₋₂ haloalkyl, C₁₋₂ alkoxy, or C₁₋₂ haloalkoxy; and the other two R⁵ are H. Embodiment A44-c is a further embodiment of Embodiment A44, wherein one of the three R⁶ is halogen, C₁₋₂ alkyl, or C₁₋₂ haloalkyl; and the other two R⁶ are H. Embodiment A44-d is a further embodiment of Embodiment A44, wherein one of the three R⁶ is F or methyl; and the other two R⁵ are H. Embodiment A44-e is a further embodiment of Embodiment A44, wherein one of the three R⁶ is methyl; and the other two R⁵ are H. Embodiment A44-e is a further embodiment of Embodiment A44, wherein one of the three R⁶ is halogen; and the other two R⁵ are H. In herein below, when Embodiment A44 is referred to, it includes Embodiment A44 and any one of its further embodiments (Embodiments A44-a to A44-e).

Embodiment A45 is a further embodiment of any one of Embodiments A1 to A8, and A15 to A42, including the further embodiments described herein, wherein T⁹ is N and each of T¹⁰, T¹¹, and T¹² is independently CR⁶. Embodiment A45-a is a further embodiment of Embodiment A45, wherein each of the three R⁶ is H. Embodiment A45-b is a further embodiment of Embodiment A45, wherein one of the three R⁶ (e.g. the R⁶ in T¹⁰ or T¹²) is halogen, —CN, C₁₋₂ alkyl, C₁₋₂ haloalkyl, C₁₋₂ alkoxy, or C₁₋₂ haloalkoxy; and the other two R⁵ are H. Embodiment A45-c is a further embodiment of Embodiment A45, wherein one of the three R⁶ (e.g. the R⁶ in T¹⁰) is C₁₋₂ alkyl or C₁₋₂ haloalkyl; and the other two R⁶ are H. Embodiment A45-d is a further embodiment of Embodiment A45, wherein one of the three R⁶ (e.g. the R⁶ in T¹⁰) is methyl; and the other two R⁵ are H.

Embodiment A45-e is a further embodiment of Embodiment A45, wherein one of the three R⁶ (e.g. the R⁶ in T¹²) is halogen, C₁₋₂ alkyl, C₁₋₂ haloalkyl, C₁₋₂ alkoxy, or C₁₋₂ haloalkoxy; and the other two R⁵ are H. Embodiment A45-f is a further embodiment of Embodiment A45, wherein one of the three R⁶ (e.g. the R⁶ in T¹²) is halogen or C₁₋₂ alkyl; and the other two R⁵ are H. Embodiment A45-g is a further embodiment of Embodiment A45, wherein one of the three R⁶ (e.g. the R⁶ in T¹²) is F; and the other two R⁵ are H. In herein below, when Embodiment A45 is referred to, it includes Embodiment A45 and any one of its further embodiments (Embodiments A45-a to A45-g).

Embodiment A46 is a further embodiment of any one of Embodiments A1 to A8, and A15 to A42, including the further embodiments described herein, wherein T¹⁰ is N and each of T⁹, T¹¹, and T¹² is independently CR⁶. Embodiment A46-a is a further embodiment of Embodiment A46, wherein each of the three R⁶ is H. Embodiment A46-b is a further embodiment of Embodiment A46, wherein one of the three R⁶ (e.g. the R⁶ in T⁹ or T¹¹) is halogen, —CN, C₁₋₂ alkyl, C₁₋₂ haloalkyl, C₁₋₂ alkoxy, or C₁₋₂ haloalkoxy; and the other two R⁵ are H. Embodiment A46-c is a further embodiment of Embodiment A46, wherein one of the three R⁶ (e.g. the R⁶ in T⁹) is C₁₋₂ alkyl or C₁₋₂ haloalkyl; and the other two R⁶ are H. Embodiment A46-d is a further embodiment of Embodiment A46, wherein one of the three R⁶ (e.g. the R⁶ in T⁹ or T¹¹) is methyl; and the other two R⁵ are H. Embodiment A46-e is a further embodiment of Embodiment A46, wherein one of the three R⁶ (e.g. the R⁶ in T¹¹) is halogen, C₁₋₂ alkyl, C₁₋₂ haloalkyl, C₁₋₂ alkoxy, or C₁₋₂ haloalkoxy; and the other two R⁵ are H. Embodiment A46-f is a further embodiment of Embodiment A46, wherein one of the three R⁶ (e.g. the R⁶ in T¹¹) is halogen or C₁₋₂ alkyl; and the other two R⁵ are H. Embodiment A46-g is a further embodiment of Embodiment A46, wherein one of the three R⁶ (e.g. the R⁶ in T¹¹) is F; and the other two R⁵ are H. In herein below, when Embodiment A46 is referred to, it includes Embodiment A46 and any one of its further embodiments (Embodiments A46-a to A46-g).

Embodiment A47 is a further embodiment of any one of Embodiments A1 to A8, and A15 to A42, including the further embodiments described herein, wherein T⁹ is N, T¹⁰ is CH or C(CH₃), T¹¹ is CH, and T¹² is CH. Embodiment A47-a is a further embodiment of Embodiment A47, wherein T⁹ is N, T¹⁰ is C(CH₃), T¹¹ is CH, and T¹² is CH. In herein below, when Embodiment A47 is referred to, it includes Embodiment A47 and any of its further embodiments (Embodiment A47-a).

Embodiment A48 is a further embodiment of any one of Embodiments A1 to A8, and A15 to A42, including the further embodiments described herein, wherein two of T⁹, T¹⁰, T¹¹, and T12 are N and the other two are each independently CR⁶.

Embodiment A48 is a further embodiment of any one of Embodiments A1 to A8, and A15 to A42, including the further embodiments described herein, wherein each of T¹⁰ and T¹¹ is N and each of T⁹ and T¹² is independently CR⁶.

Embodiment A49 is a further embodiment of any one of Embodiments A1 to A8, and A15 to A48, including the further embodiments described herein, wherein each R⁶ is independently H, halo, C₁₋₂ alkyl, or C₁₋₂ haloalkyl.

Embodiment A50 is a further embodiment of any one of Embodiments A1 to A8, and A15 to A48, including the further embodiments described herein, wherein each R⁶ is independently H, halo, or C₁₋₂ alkyl.

Embodiment A51 is a further embodiment of any one of Embodiments A1 to A8, and A15 to A48, including the further embodiments described herein, wherein each R⁶ is independently H or halo.

Embodiment A52 is a further embodiment of any one of Embodiments A1 to A8, and A15 to A48, including the further embodiments described herein, wherein each R⁶ is independently H or C₁₋₂ alkyl.

Embodiment A53 is a further embodiment of any one of Embodiments A1 to A6, A9, A10, A15 to A33, and A34 to A42, including the further embodiments described herein, wherein each of T¹³, T¹⁴, T¹⁵, and T¹⁶ is independently CR⁷.

Embodiment A54 is a further embodiment of any one of Embodiments A1 to A6, A9, A10, A15 to A33, and A34 to A42, including the further embodiments described herein, wherein one of T¹³, T¹⁴, T¹⁵, and T¹⁶ is N and the other three are each independently CR⁷.

Embodiment A55 is a further embodiment of any one of Embodiments A1 to A6, A9, A10, A15 to A33, and A34 to A42, including the further embodiments described herein, wherein T¹³ is N and each of T¹⁴, T¹⁵, and T¹⁶ is independently CR⁷.

Embodiment A56 is a further embodiment of any one of Embodiments A1 to A6, A9, A10, A15 to A33, and A34 to A42, including the further embodiments described herein, wherein two of T¹³, T¹⁴, T¹⁵, and T¹⁶ are N and the other two are each independently CR⁷.

Embodiment A57 is a further embodiment of any one of Embodiments A1 to A6, A9, A10, A15 to A33, A34 to A42, and A53 to A56, including the further embodiments described herein, wherein each R⁷ is independently H, halo, C₁₋₂ alkyl, or C₁₋₂ haloalkyl.

Embodiment A58 is a further embodiment of any one of Embodiments A1 to A6, A9, A10, A15 to A33, A34 to A42, and A53 to A57, including the further embodiments described herein, wherein each R⁷ is independently H, halo, or C₁₋₂ alkyl.

Embodiment A59 is a further embodiment of any one of Embodiments A1 to A6, A9, A10, A15 to A33, A34 to A42, and A53 to A57, including the further embodiments described herein, wherein each R⁷ is independently H or halo.

Embodiment A60 is a further embodiment of any one of Embodiments A1 to A6, A9, A10, A15 to A33, A34 to A42, and A53 to A57, including the further embodiments described herein, wherein each R⁷ is independently H or C₁₋₂ alkyl.

Embodiment A61 is a further embodiment of any one of Embodiments A1 to A6, A9, A10, A15 to A33, A34 to A42, and A53 to A57, including the further embodiments described herein, wherein each R⁷ is H.

Embodiment A62 is a further embodiment of any one of Embodiments A1 to A6, and A11 to A61, including the further embodiments described herein, wherein each of T¹⁷, T¹⁸, and T¹⁹ is independently CR⁸.

Embodiment A63 is a further embodiment of any one of Embodiments A1 to A6, and A11 to A33, including the further embodiments described herein, wherein one of T¹⁷, T¹⁸, and T¹⁹ is N, and the other two are independently CR⁸.

Embodiment A64 is a further embodiment of any one of Embodiments A1 to A6, A11 to A33, A62, and A63, including the further embodiments described herein, wherein each R⁸ is independently H, halo, C₁₋₂ alkyl, or C₁₋₂ haloalkyl.

Embodiment A65 is a further embodiment of any one of Embodiments A1 to A6, A11 to A33, A62, and A63, including the further embodiments described herein, wherein each R⁸ is independently H, halo, or C₁₋₂ alkyl.

Embodiment A66 is a further embodiment of any one of Embodiments A1 to A6, A11 to A33, A62, and A63, including the further embodiments described herein, wherein each R⁸ is independently H or C₁₋₂ alkyl.

Embodiment A67 is a further embodiment of any one of Embodiments A1 to A6, A11 to A33, A62, and A63, including the further embodiments described herein, wherein each R⁸ is independently H or halo.

Embodiment A68 is a further embodiment of any one of Embodiments A1 to A6, A11 to A33, A62, and A63, including the further embodiments described herein, wherein each R⁸ is H.

Embodiment A69 is a further embodiment of any one of Embodiments A1 to A6, and A11, A12, A15 to A33, and A62 to A68, including the further embodiments described herein, wherein each of T²⁰, T²¹, and T²² is independently CR⁹.

Embodiment A70 is a further embodiment of any one of Embodiments A1 to A6, and A11, A12, A15 to A33, and A62 to A68, including the further embodiments described herein, wherein one of T²⁰, T²¹, and T¹¹ is N, and the other two are each independently CR⁹.

Embodiment A71 is a further embodiment of any one of Embodiments A1 to A6, and A11, A12, A15 to A33, and A62 to A68, including the further embodiments described herein, wherein T²⁰ is N, and each of T²¹ and T²² is independently CR⁹.

Embodiment A72 is a further embodiment of any one of Embodiments A1 to A6, and A11, A12, A15 to A33, and A62 to A71, including the further embodiments described herein, wherein each R⁹ is independently H, halo, C₁₋₂ alkyl, or C₁₋₂ haloalkyl.

Embodiment A73 is a further embodiment of any one of Embodiments A1 to A6, and A11, A12, A15 to A33, and A62 to A71, including the further embodiments described herein, wherein each R⁹ is independently H, halo, or C₁₋₂ alkyl.

Embodiment A74 is a further embodiment of any one of Embodiments A1 to A6, and A11, A12, A15 to A33, and A62 to A71, including the further embodiments described herein, wherein each R⁹ is independently H or halo.

Embodiment A75 is a further embodiment of any one of Embodiments A1 to A6, and A11, A12, A15 to A33, and A62 to A71, including the further embodiments described herein, wherein each R⁹ is independently H or C₁₋₂ alkyl.

Embodiment A76 is a further embodiment of any one of Embodiments A1 to A6, and A11, A12, A15 to A33, and A69 to A71, including the further embodiments described herein, wherein each R⁹ is H.

Embodiment A77 is a further embodiment of any one of Embodiments A1 to A6, and A13, A14, A15 to A33, and A62 to A76, including the further embodiments described herein, wherein t3 is 1.

Embodiment A78 is a further embodiment of any one of Embodiments A1 to A6, A13, A14, A15 to A33, and A62 to A76, including the further embodiments described herein, wherein t3 is 2 (e.g. wherein the compound has the structure of Formula VII or Formula VIIa and t3 is 2, or a pharmaceutically acceptable salt thereof). Embodiment A78-a is a further embodiment of Embodiment A78, wherein each of T¹⁷ and T¹⁸ is independently CR⁸; and T¹⁹ is N. Embodiment A78-b is a further embodiment of Embodiment A78, wherein each of T¹⁷ and T¹⁸ is CH; and T¹⁹ is N. Embodiment A78-c is a further embodiment of Embodiment A78, wherein each of T¹⁷ and T¹⁸ is independently CR⁸; T¹⁹ is N, and R^{A} is C(═O)OH. Embodiment A78-d is a further embodiment of Embodiment A78, wherein each of T¹⁷ and T¹⁸ is independently CH; T¹⁹ is N, and R^{A} is C(═O)OH. In herein below, when Embodiment A78 is referred to, it includes Embodiment A78 and any of its further embodiments (Embodiments A78-a to A78-d).

Embodiment A79 is a further embodiment of any one of Embodiments A1 to A6, and A13, A14, A15 to A33, and A62 to A78, including the further embodiments described herein, wherein t4 is 0, 1, or 2; and each R¹⁰ is independently halogen, —OH, C₁₋₄ alkyl, C₁₋₄ hydroxylalkyl, C₁₋₄ haloalkyl, C₁₋₄ alkoxy, C₁₋₄ haloalkoxy, C₃₋₄ cycloalkyl, or (C₃₋₄ cycloalkyl)-C₁₋₄ alkyl-.

Embodiment A80 is a further embodiment of any one of Embodiments A1 to A6, and A13, A14, A15 to A33, and A62 to A78, including the further embodiments described herein, wherein t4 is 0 or 1; and each R¹⁰ is halogen, —OH, C₁₋₄ alkyl, C₁₋₄ hydroxylalkyl, C₁₋₄ haloalkyl, C₁₋₄ alkoxy, C₁₋₄ haloalkoxy, C₃₋₄ cycloalkyl, or (C₃₋₄ cycloalkyl)-C₁₋₄ alkyl-.

Embodiment A81 is a further embodiment of any one of Embodiments A1 to A80 including the further embodiments described herein, wherein R^{A} is —C(═O)—OH.

Embodiment A82 is a further embodiment of any one of Embodiments A1 to A80 including the further embodiments described herein, wherein R^{A} is —C(═O)—NH₂.

Embodiment A83 is a further embodiment of any one of Embodiments A1 to A80 including the further embodiments described herein, wherein R^{A} is —OH.

Embodiment A84 is a compound selected from:
5-{4-[(1-{[4-(propan-2-yl)phenyl]carbamoyl}-D-prolyl)amino]phenyl}pyridine-2-carboxylic acid;
6-{4-[(1-{[4-(propan-2-yl)phenyl]carbamoyl}-D-prolyl)amino]phenyl}pyridine-3-carboxylic acid;
4′-[(1-{[4-(propan-2-yl)phenyl]carbamoyl}-D-prolyl)amino][1,1′-biphenyl]-3-carboxylic acid; 4′-[(1-{[4-(propan-2-yl)phenyl]carbamoyl}-D-prolyl)amino][1,1′-biphenyl]-4-carboxylic acid;
4-{6-[(1-{[4-(propan-2-yl)phenyl]carbamoyl}-D-prolyl)amino]pyridin-3-yl}benzoic acid;
3′-fluoro-4′-[(1-{[4-(propan-2-yl)phenyl]carbamoyl}-D-prolyl)amino][1,1′-biphenyl]-4-carboxylic acid;
4′-({1-[(4-cyclopropylphenyl)carbamoyl]-D-prolyl}amino)[1,1′-biphenyl]-4-carboxylic acid;
2-{4-[(1-{[4-(propan-2-yl)phenyl]carbamoyl}-D-prolyl)amino]phenyl}pyrimidine-5-carboxylic acid;
6-{4-[(1-{[4-(propan-2-yl)phenyl]carbamoyl}-D-prolyl)amino]phenyl}pyridine-2-carboxylic acid;
6-[(1-{[4-(propan-2-yl)phenyl]carbamoyl}-D-prolyl)amino]naphthalene-2-carboxylic acid;
8-methyl-6-[(1-{[4-(propan-2-yl)phenyl]carbamoyl}-D-prolyl)amino]quinoline-2-carboxylic acid;
4′-[(1-{[4-(prop-1-en-2-yl)phenyl]carbamoyl}-D-prolyl)amino][1,1′-biphenyl]-4-carboxylic acid;
4′-({l-[(4-chlorophenyl)carbamoyl]-D-prolyl}amino)[1,1′-biphenyl]-4-carboxylic acid;
4-{4-[(1-{[4-(propan-2-yl)phenyl]carbamoyl}-D-prolyl)amino]phenyl}pyridine-2-carboxylic acid; and
3′,5′-difluoro-4′-[(1-{[4-(propan-2-yl)phenyl]carbamoyl}-D-prolyl)amino][1,1′-biphenyl]-4-carboxylic acid,
   or a pharmaceutically acceptable salt thereof.

Embodiment A85 is a compound selected from:
4′-[(1-{[4-(propan-2-yl)phenyl]carbamoyl}-D-prolyl)amino][1,1′-biphenyl]-4-carboxylic acid;
5-{4-[(1-{[3-methyl-4-(propan-2-yl)phenyl]carbamoyl}-D-prolyl)amino]phenyl}pyridine-2-carboxylic acid;
6-methyl-5-{4-[(1-{[4-(propan-2-yl)phenyl]carbamoyl}-D-prolyl)amino]phenyl}pyridine-2-carboxylic acid;
3-methoxy-4′-[(1-{[4-(propan-2-yl)phenyl]carbamoyl}-D-prolyl)amino][1,1′-biphenyl]-4-carboxylic acid;
5-{4-[(1-{[3-fluoro-4-(propan-2-yl)phenyl]carbamoyl}-D-prolyl)amino]phenyl}-6-methylpyridine-2-carboxylic acid;
3-fluoro-4′-[(1-{[4-(propan-2-yl)phenyl]carbamoyl}-D-prolyl)amino][1,1′-biphenyl]-4-carboxylic acid;
4-{6-[(1-{[4-(trifluoromethyl)phenyl]carbamoyl}-D-prolyl)amino]pyridin-3-yl}benzoic acid;
4′-[(1-{[3-fluoro-4-(propan-2-yl)phenyl]carbamoyl}-D-prolyl)amino]-3-methoxy[1,1′-biphenyl]-4-carboxylic acid;
4-{3-fluoro-5-[(1-{[4-(propan-2-yl)phenyl]carbamoyl}-D-prolyl)amino]pyridin-2-yl}benzoic acid; and
6-methyl-5-{4-[(1-{[3-methyl-4-(trifluoromethyl)phenyl]carbamoyl}-D-prolyl)amino]phenyl}pyridine-2-carboxylic acid,
   or a pharmaceutically acceptable salt thereof.

Embodiment A86 is a compound selected from Examples 1 to 229 (e.g. a compound selected from Examples 16-38), or a pharmaceutically acceptable salt thereof (or its free acid form or a pharmaceutically acceptable salt of its free acid form where an example is a salt).

Embodiment 1 is a pharmaceutical composition comprising a compound of any one of Embodiments A1 to A86 including the further embodiments described herein and a pharmaceutically acceptable excipient.

Embodiment C1 is a method for treating or preventing a condition, disease, or disorder in a patient comprising administering to the patient a compound of any one of Embodiments A1 to A86 including the further embodiments described herein, wherein the condition, disease, or disorder is selected from the group consisting of diabetes [e.g. Type 1 diabetes mellitus (T1D), Type 2 diabetes mellitus (T2DM), including pre-diabetes], idiopathic T1D (Type 1b), latent autoimmune diabetes in adults (LADA), early-onset T2DM (EOD), youth-onset atypical diabetes (YOAD), maturity onset diabetes of the young (MODY), malnutrition-related diabetes, gestational diabetes, hyperglycemia, insulin resistance, hepatic insulin resistance, impaired glucose tolerance, diabetic neuropathy, diabetic nephropathy, kidney disease [e.g., acute kidney disorder, tubular dysfunction, proinflammatory changes to the proximal tubules, or chronic kidney disease (CKD)], diabetic retinopathy, adipocyte dysfunction, visceral adipose deposition, sleep apnea [e.g. obstructive sleep apnea (OSA)], obesity (including hypothalamic obesity and monogenic obesity) and related comorbidities (e.g., osteoarthritis and urine incontinence), eating disorders (including binge eating syndrome, bulimia nervosa, and syndromic obesity such as Prader-Willi and Bardet-Biedl syndromes), weight gain such as weight gain caused by use of other agents (e.g., caused by use of steroids and/or antipsychotics, or caused by treatment of depression, or caused by use of agents on cognitive function), overweight, excessive sugar craving, dyslipidemia [including hyperlipidemia, hypertriglyceridemia, increased total cholesterol, high LDL (low-density lipoprotein) cholesterol, and low HDL (high-density lipoprotein) cholesterol], hyperinsulinemia, nonalcoholic fatty liver disease [NAFLD, including related diseases such as steatosis, nonalcoholic steatohepatitis (NASH), fibrosis, cirrhosis, and hepatocellular carcinoma], cardiovascular disease, atherosclerosis (including coronary artery disease), peripheral vascular disease, hypertension, endothelial dysfunction, impaired vascular compliance, heart failure [e.g. congestive heart failure, heart failure with preserved ejection fraction (HFpEF), heart failure with reduced ejection fraction (HFrEF)], myocardial infarction (e.g. necrosis and apoptosis), stroke, hemorrhagic stroke, ischemic stroke, traumatic brain injury, pulmonary hypertension, restenosis after angioplasty, intermittent claudication, post-prandial lipemia, metabolic acidosis, ketosis, arthritis, osteoporosis, osteoarthritis, Parkinson's disease, left ventricular hypertrophy, peripheral arterial disease (PAD), macular degeneration, cataract, glomerulosclerosis, chronic renal failure, metabolic syndrome, syndrome X, premenstrual syndrome, angina pectoris, thrombosis, atherosclerosis, transient ischemic attacks, vascular restenosis, impaired glucose metabolism, conditions of impaired fasting plasma glucose, hyperuricemia, gout, erectile dysfunction, skin and connective tissue disorders, psoriasis, foot ulcerations, ulcerative colitis, hyper apo B lipoproteinemia, Alzheimer's Disease, schizophrenia, impaired cognition, inflammatory bowel disease, short bowel syndrome, Crohn's disease, colitis, irritable bowel syndrome, polycystic ovary syndrome (PCOS), and addiction (e.g., addition to alcohol, nicotine, and/or drug); or a method for weight management (e.g. chronic weight management) of a human comprising administering to the human a compound of any one of Embodiments A1 to A86 including the further embodiments described herein.

As used herein, treating diabetes (e.g. T2DM) in a diabetic patient (e.g. a patient with T2DM) includes, among other things, improving glycemic control.

Embodiment C2 is a further embodiment of Embodiment C1, wherein the condition, disease, or disorder is selected from the group consisting of obesity, weight gain, T2DM, Heart Failure (e.g. HFpEF and HFrEF); CKD; NAFLD, NASH, atherosclerosis, PAD, obstructive sleep apnea, diabetic retinopathy, and diabetic neuropathy.

Embodiment C3 is a further embodiment of Embodiment C1, wherein the method is for preventing weight gain.

Embodiment C4 is a further embodiment of Embodiment C1, wherein the method is for preventing obesity.

Embodiment C5 is a further embodiment of Embodiment C1, wherein the method is for treating obesity.

Embodiment C6 is a further embodiment of Embodiment C1, wherein the method is for weight management, for example chronic weight management, of a human. In some further embodiments, the human is obese or overweight when the weight management (e.g. chronic weight management) is initiated; and in such a situation, the weight management (e.g. chronic weight management) is also a method for treating obesity or overweight. In some further embodiments, the human is obese when the weight management (e.g. chronic weight management) treatment is initiated; and in such a situation, the weight management (e.g. chronic weight management) is also a method for treating obesity.

Embodiment D1 is use of a compound of any one of Embodiments A1 to A86 including the further embodiments described herein for treating or preventing a condition, disease, or disorder, or use of a compound in manufacturing a medicament for treating or preventing a condition, disease, or disorder, wherein the condition, disease, or disorder is selected from the group consisting of diabetes [e.g. Type 1 diabetes mellitus (T1D), Type 2 diabetes mellitus (T2DM), including pre-diabetes], idiopathic T1D (Type 1b), latent autoimmune diabetes in adults (LADA), early-onset T2DM (EOD), youth-onset atypical diabetes (YOAD), maturity onset diabetes of the young (MODY), malnutrition-related diabetes, gestational diabetes, hyperglycemia, insulin resistance, hepatic insulin resistance, impaired glucose tolerance, diabetic neuropathy, diabetic nephropathy, kidney disease [e.g., acute kidney disorder, tubular dysfunction, proinflammatory changes to the proximal tubules, or chronic kidney disease (CKD)], diabetic retinopathy, adipocyte dysfunction, visceral adipose deposition, sleep apnea [e.g. obstructive sleep apnea (OSA)], obesity (including hypothalamic obesity and monogenic obesity) and related comorbidities (e.g., osteoarthritis and urine incontinence), eating disorders (including binge eating syndrome, bulimia nervosa, and syndromic obesity such as Prader-Willi and Bardet-Biedl syndromes), weight gain such as weight gain caused by use of other agents (e.g., caused by use of steroids and/or antipsychotics, or caused by treatment of depression, or caused by use of agents on cognitive function), overweight, excessive sugar craving, dyslipidemia [including hyperlipidemia, hypertriglyceridemia, increased total cholesterol, high LDL (low-density lipoprotein) cholesterol, and low HDL (high-density lipoprotein) cholesterol], hyperinsulinemia, nonalcoholic fatty liver disease [NAFLD, including related diseases such as steatosis, nonalcoholic steatohepatitis (NASH), fibrosis, cirrhosis, and hepatocellular carcinoma], cardiovascular disease, atherosclerosis (including coronary artery disease), peripheral vascular disease, hypertension, endothelial dysfunction, impaired vascular compliance, heart failure [e.g. congestive heart failure, heart failure with preserved ejection fraction (HFpEF), heart failure with reduced ejection fraction (HFrEF)], myocardial infarction (e.g. necrosis and apoptosis), stroke, hemorrhagic stroke, ischemic stroke, traumatic brain injury, pulmonary hypertension, restenosis after angioplasty, intermittent claudication, post-prandial lipemia, metabolic acidosis, ketosis, arthritis, osteoporosis, osteoarthritis, Parkinson's disease, left ventricular hypertrophy, peripheral arterial disease (PAD), macular degeneration, cataract, glomerulosclerosis, chronic renal failure, metabolic syndrome, syndrome X, premenstrual syndrome, angina pectoris, thrombosis, atherosclerosis, transient ischemic attacks, vascular restenosis, impaired glucose metabolism, conditions of impaired fasting plasma glucose, hyperuricemia, gout, erectile dysfunction, skin and connective tissue disorders, psoriasis, foot ulcerations, ulcerative colitis, hyper apo B lipoproteinemia, Alzheimer's Disease, schizophrenia, impaired cognition, inflammatory bowel disease, short bowel syndrome, Crohn's disease, colitis, irritable bowel syndrome, polycystic ovary syndrome (PCOS), and addiction (e.g., addition to alcohol, nicotine, and/or drug); or use of a compound of any one of Embodiments A1 to A86 including the further embodiments described herein for weight management (e.g. chronic weight management).

Embodiment D2 is a further embodiment of Embodiment D1, wherein the condition, disease, or disorder is selected from the group consisting of obesity, weight gain, T2DM, Heart Failure (e.g. HFpEF and HFrEF); CKD; NAFLD, NASH, atherosclerosis, PAD, obstructive sleep apnea, diabetic retinopathy, and diabetic neuropathy.

Embodiment D3 is a further embodiment of Embodiment D1, wherein the use of a compound of any one of Embodiments A1 to A86 including the further embodiments described herein is for preventing weight gain.

Embodiment D4 is a further embodiment of Embodiment D1, wherein the use of a compound of any one of Embodiments A1 to A86 including the further embodiments described herein is in manufacturing a medicament for preventing weight gain.

Embodiment D5 is a further embodiment of Embodiment D1, wherein the use of a compound of any one of Embodiments A1 to A86 including the further embodiments described herein is for treating obesity.

Embodiment D6 is a further embodiment of Embodiment D1, wherein the use of a compound of any one of Embodiments A1 to A86 including the further embodiments described herein is in manufacturing a medicament for obesity.

Embodiment D7 is a further embodiment of Embodiment D1, wherein the use of a compound of any one of Embodiments A1 to A86 including the further embodiments described herein is in manufacturing a medicament for weight management, for example chronic weight management of a human. In some further embodiments, the human is obese or overweight when the weight management (e.g. chronic weight management) is initiated; and in such a situation, the weight management is also a method for treating obesity or overweight. In some further embodiments, the human is obese when the weight management (e.g. chronic weight management) treatment is initiated; and in such a situation, the weight management is also a method for treating obesity.

Embodiment E1 is a compound of any one of Embodiments A1 to A86 including the further embodiments described herein for use in a method for treating or preventing a condition, disease, or disorder in a patient, wherein the condition, disease, or disorder is selected from the group consisting of diabetes [e.g. Type 1 diabetes mellitus (T1 D), Type 2 diabetes mellitus (T2DM), including pre-diabetes], idiopathic T1D (Type 1b), latent autoimmune diabetes in adults (LADA), early-onset T2DM (EOD), youth-onset atypical diabetes (YOAD), maturity onset diabetes of the young (MODY), malnutrition-related diabetes, gestational diabetes, hyperglycemia, insulin resistance, hepatic insulin resistance, impaired glucose tolerance, diabetic neuropathy, diabetic nephropathy, kidney disease [e.g., acute kidney disorder, tubular dysfunction, proinflammatory changes to the proximal tubules, or chronic kidney disease (CKD)], diabetic retinopathy, adipocyte dysfunction, visceral adipose deposition, sleep apnea [e.g. obstructive sleep apnea (OSA)], obesity (including hypothalamic obesity and monogenic obesity) and related comorbidities (e.g., osteoarthritis and urine incontinence), eating disorders (including binge eating syndrome, bulimia nervosa, and syndromic obesity such as Prader-Willi and Bardet-Biedl syndromes), weight gain such as weight gain caused by use of other agents (e.g., caused by use of steroids and/or antipsychotics, or caused by treatment of depression, or caused by use of agents on cognitive function), overweight, excessive sugar craving, dyslipidemia [including hyperlipidemia, hypertriglyceridemia, increased total cholesterol, high LDL (low-density lipoprotein) cholesterol, and low HDL (high-density lipoprotein) cholesterol], hyperinsulinemia, nonalcoholic fatty liver disease [NAFLD, including related diseases such as steatosis, nonalcoholic steatohepatitis (NASH), fibrosis, cirrhosis, and hepatocellular carcinoma], cardiovascular disease, atherosclerosis (including coronary artery disease), peripheral vascular disease, hypertension, endothelial dysfunction, impaired vascular compliance, heart failure [e.g. congestive heart failure, heart failure with preserved ejection fraction (HFpEF), heart failure with reduced ejection fraction (HFrEF)], myocardial infarction (e.g. necrosis and apoptosis), stroke, hemorrhagic stroke, ischemic stroke, traumatic brain injury, pulmonary hypertension, restenosis after angioplasty, intermittent claudication, post-prandial lipemia, metabolic acidosis, ketosis, arthritis, osteoporosis, osteoarthritis, Parkinson's disease, left ventricular hypertrophy, peripheral arterial disease (PAD), macular degeneration, cataract, glomerulosclerosis, chronic renal failure, metabolic syndrome, syndrome X, premenstrual syndrome, angina pectoris, thrombosis, atherosclerosis, transient ischemic attacks, vascular restenosis, impaired glucose metabolism, conditions of impaired fasting plasma glucose, hyperuricemia, gout, erectile dysfunction, skin and connective tissue disorders, psoriasis, foot ulcerations, ulcerative colitis, hyper apo B lipoproteinemia, Alzheimer's Disease, schizophrenia, impaired cognition, inflammatory bowel disease, short bowel syndrome, Crohn's disease, colitis, irritable bowel syndrome, polycystic ovary syndrome (PCOS), and addiction (e.g., addition to alcohol, nicotine, and/or drug); or is a compound of any one of Embodiments A1 to A86 including the further embodiments described herein for use in a method for weight management (e.g. chronic weight management).

Embodiment E2 is a further embodiment of Embodiment E1, wherein the condition, disease, or disorder is selected from the group consisting of obesity, weight gain, T2DM, Heart Failure (e.g. HFpEF and HFrEF); CKD; NAFLD, NASH, atherosclerosis, PAD, obstructive sleep apnea, diabetic retinopathy, and diabetic neuropathy.

Embodiment E3 is a further embodiment of Embodiment E1, wherein a compound of any one of Embodiments A1 to A86 including the further embodiments described herein is for use in a method for preventing weight gain.

Embodiment E4 is a further embodiment of Embodiment E1, wherein a compound of any one of Embodiments A1 to A86 including the further embodiments described herein is for use in a method for treating obesity.

Embodiment E5 is a further embodiment of Embodiment E1, wherein a compound of any one of Embodiments A1 to A86 including the further embodiments described herein is for use in a method for weight management (e.g. chronic weight management). In some further embodiments, the human is obese or overweight when the weight management (e.g. chronic weight management) is initiated; and in such a situation, the weight management is also a method for treating obesity or overweight. In some further embodiments, the human is obese when the weight management (e.g. chronic weight management) treatment is initiated; and in such a situation, the weight management is also a method for treating obesity.

Embodiment F1 is a method for modulating (e.g. antagonizing) a GIPR (either in vitro or in vivo), comprising contacting (including incubating) the GIPR with a compound of any one of Embodiments A1 to A86 including the further embodiments described herein.

Embodiment F2 is a further embodiment of Embodiment F1, wherein said modulating is antagonizing.

It is to be understood that this invention is not limited to specific synthetic methods of preparation described in the schemes herein. It is also to be understood that the terminology used herein is for the purpose of describing particular embodiments only and is not intended to be limiting. In this specification and in the claims that follow, reference will be made to a number of terms that shall be defined to have the following meanings:

As used herein in the specification, “a” or “an” may mean one or more. As used herein in the claim(s), when used in conjunction with the word “comprising”, the words “a” or “an” may mean one or more than one. As used herein “another” may mean at least a second or more.

The term “about” refers to a relative term denoting an approximation of plus or minus 10% of the nominal value to which it refers, in one embodiment, to plus or minus 5%, in another embodiment, to plus or minus 2%. For the field of this disclosure, this level of approximation is appropriate unless the value is specifically stated to require a tighter range.

“Compound” when used herein includes any pharmaceutically acceptable derivative or variation, including conformational isomers (e.g., cis and trans isomers) and all optical isomers (e.g., enantiomers and diastereomers), racemic, diastereomeric and other mixtures of such isomers, as well as solvates, hydrates, isomorphs, polymorphs, tautomers, esters, salt forms, and prodrugs.

As used herein, a wavy line,

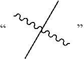

denotes a point of attachment of a substituent to another group.

The term “alkyl” means an acyclic, saturated aliphatic hydrocarbon group which may be straight/linear or branched. Examples of such groups include, but are not limited to, methyl, ethyl, n-propyl, isopropyl, butyl, sec-butyl, isobutyl and tert-butyl. The carbon atom content of alkyl and various other hydrocarbon-containing moieties is indicated by a prefix designating a lower and upper number of carbon atoms in the moiety, that is, the prefix Cᵢ₋ⱼ indicates a moiety of the integer “i” to the integer “j” carbon atoms, inclusive. Thus, for example, C₁₋₈ alkyl refers to alkyl of one to eight carbon atoms, inclusive; for another example, C₁₋₆ alkyl refers to alkyl of one to six carbon atoms, inclusive; for yet another example, C₁₋₄ alkyl refers to alkyl of one to four carbon atoms, inclusive. Representative examples of C₁₋₄ alkyl include methyl, ethyl, n-propyl, isopropyl, n-butyl, sec-butyl, isobutyl, and tert-butyl. For another example, C₁₋₂ alkyl refers to alkyl of one to two carbon atoms, inclusive (i.e., methyl or ethyl). The alkyl group optionally can be substituted by 1 or more (e.g., 1 to 5) suitable substituents, when so specified.

At various places in the present specification, substituents of compounds of the invention are disclosed in groups or in ranges. It is specifically intended that the invention include each and every individual sub-combination of the members of such groups and ranges. For example, the term “C₁₋₄ alkyl” is specifically intended to include C₁ alkyl (methyl), C₂ alkyl (ethyl), C₃ alkyl, and C4 alkyl. For another example, the term “4- to 7-membered heterocycloalkyl” is specifically intended to include any 4-, 5-, 6-, or 7-membered heterocycloalkyl group. For yet another example, the term “C₃₋₆ cycloalkyl” is specifically intended to include any saturated or unsaturated, non-aromatic, monocyclic or polycyclic (such as bicyclic) hydrocarbon rings of 3, 4, 5, or 6 ring-forming carbon atoms.

As used herein, the term “n-membered”, where n is an integer, typically describes the number of ring-forming atoms in a moiety where the number of ring-forming atoms is n. For example, piperidinyl is an example of a 6-membered heterocycloalkyl ring and pyrrolindinyl is an example of a 5-membered heterocycloalkyl group.

As used herein, the term “alkoxy” or “alkyloxy” refers to an —O-alkyl group. For example, the term “C₁₋₄ alkoxy” or “C₁₋₄ alkyloxy” refers to an —O—(C₁₋₄ alkyl) group; For another example, the term “C₁₋₂ alkoxy” or “C₁₋₂ alkyloxy” refers to an —O—(C₁₋₂ alkyl) group. Examples of alkoxy include methoxy, ethoxy, propoxy (e.g., n-propoxy and isopropoxy), tert-butoxy, and the like. The alkoxy or alkyloxy group optionally can be substituted by 1 or more (e.g., 1 to 5) suitable substituents when so specified.

The term “halo” or “halogen” as used herein, means —F, —Cl, —Br, or —I.

As used herein, the term “haloalkyl” refers to an alkyl group having one or more halogen substituents (up to perhaloalkyl, i.e., every hydrogen atom of the alkyl group has been replaced by a halogen atom). For example, the term “C₁₋₄ haloalkyl” refers to a C₁₋₄ alkyl group having one or more halogen substituents (up to perhaloalkyl, i.e., every hydrogen atom of the alkyl group has been replaced by a halogen atom); and the term “C₁₋₂ haloalkyl” refers to a C₁₋₂ alkyl group (i.e., methyl or ethyl) having one or more halogen substituents (up to perhaloalkyl, i.e., every hydrogen atom of the alkyl group has been replaced by a halogen atom). Examples of haloalkyl groups include —CF₃, —CHF₂, —CH₂F, —CH₂CF₃, —C₂F₅, —CH₂Cl and the like.

“Fluoroalkyl” as used herein means an alkyl as defined herein substituted with one or more fluoro (—F) substituents (up to perfluoroalkyl, i.e., every hydrogen atom of the alkyl group has been replaced by a fluorine atom). The term “C₁₋₂ fluoroalkyl” refers to a C₁₋₂ alkyl group (i.e., methyl or ethyl) having one or more fluorine substituents (up to perfluoroalkyl, i.e., every hydrogen atom of the alkyl group has been replaced by a fluorine atom); and the term “C₁ fluoroalkyl” refers to methyl having 1, 2, or 3 fluorine substituents. Examples of C₁ fluoroalkyl include fluoromethyl, difluoromethyl and trifluoromethyl; some examples of C₂ fluoroalkyl include 1-fluoroethyl, 2-fluoroethyl, 2,2-difluoroethyl, 1,2-difluoroethyl, 2,2,2-trifluoroethyl, 1,1,2-trifluoroethyl, and the like.

As used here, the term “haloalkoxy” refers to an —O-haloalkyl group. For example, the term “C₁₋₄ haloalkoxy” refers to an —O—(C₁₋₄ haloalkyl) group; and the term “C₁₋₂ haloalkoxy” refers to an —O—(C₁₋₂ haloalkyl) group. For yet another example, the term “C₁ haloalkoxy” refers to a methoxy group having one, two, or three halogen substituents. An example of haloalkoxy is —OCF₃ or —OCHF₂.

As used here, the term “fluoroalkoxy” refers to an —O-fluoroalkyl group. For example, the term “C₁₋₂ fluoroalkoxy” refers to an —O—(C₁₋₂ fluoroalkyl) group; and the term “C₁ fluoroalkoxy” refers to an —O—(C₁ fluoroalkyl) group. Examples of C₁ fluoroalkoxy include —O—CH₂F, —O—CHF₂, and —O—CF₃. Some examples of C₂ fluoroalkoxy include —O—CH₂CHF₂, —O—CH₂—CHF₂, —O—CH₂CF₃, —O—CF₂CH₃, and —O—CF₂CF₃.

As used herein, the term “hydroxylalkyl” or “hydroxyalkyl” refers to an alkyl group having one or more (e.g., 1, 2, or 3) OH substituents. The term “C₁₋₄ hydroxylalkyl” or “C₁₋₄ hydroxyalkyl” refers to a C₁₋₄ alkyl group having one or more (e.g., 1, 2, or 3) OH substituents; and the term “C₁₋₂ hydroxylalkyl” or “C₁₋₂ hydroxyalkyl” refers to a C₁₋₂ alkyl group having one or more (e.g., 1, 2, or 3) OH substituents. An example of hydroxylalkyl is —CH₂OH or —CH₂CH₂OH.

As used herein, the term “cyanoalkyl” refers to an alkyl group having one or more (e.g., 1, 2, or 3) —CN (i.e. —C≡N or cyano) substituents. For example, The term “C₁₋₄ cyanoalkyl” refers to a C₁₋₄ alkyl group having one or more (e.g., 1, 2, or 3) —CN substituents. An example of cyanoalkyl is —CH₂—CN or —CH₂CH₂—CN.

As used herein, the term “alkenyl” refers to aliphatic hydrocarbons having at least one carbon-carbon double bond, including straight chains and branched chains having at least one carbon-carbon double bond. In some embodiments, the alkenyl group has 2 to 20 carbon atoms, 2 to 10 carbon atoms, 2 to 6 carbon atoms, 3 to 6 carbon atoms, or 2 to 4 carbon atoms. For example, as used herein, the term “C₂₋₈ alkenyl” refers to straight or branched chain unsaturated radicals (having at least one carbon-carbon double bond) of 2 to 8 carbon atoms; the term “C₃₋₆ alkenyl” refers to straight or branched chain unsaturated radicals (having at least one carbon-carbon double bond) of 3 to 6 carbon atoms; and the term “C₃₋₄ alkenyl” refers to straight or branched chain unsaturated radicals (having at least one carbon-carbon double bond) of 3 to 4 carbon atoms. Examples of “C₃₋₆ alkenyl” include, but are not limited to, prop-2-en-1-yl, prop-1-en-2-yl, but-2-en-1-yl, but-2-en-2-yl, 2-methylbut-2-en-1-yl, and the like. An alkenyl group optionally can be substituted by one or more (e.g. 1 to 5) suitable substituents. When the compounds of Formula I contain an alkenyl group, the alkenyl group may exist as the pure E form, the pure Z form, or any mixture thereof when applicable.

As used herein, the term “cycloalkyl” refers to saturated or unsaturated, non-aromatic, monocyclic or polycyclic (such as bicyclic) hydrocarbon rings (e.g., monocyclics such as cyclopropyl, cyclobutyl, cyclopentyl, cyclohexyl, cycloheptyl, cyclooctyl, cyclononyl, or bicyclics including spiro, fused, or bridged systems (such as bicyclo[1.1.1]pentanyl, bicyclo[2.2.1]heptanyl, bicyclo[3.2.1]octanyl or bicyclo[5.2.0]nonanyl, decahydronaphthalenyl, etc.). The cycloalkyl group has 3 to 15 (e.g., 3 to 14, 3 to 10, 3 to 6, 3 to 4, or 4 to 6) carbon atoms. In some embodiments the cycloalkyl may optionally contain one, two, or more non-cumulative non-aromatic double or triple bonds and/or one to three oxo groups. In some embodiments, the bicycloalkyl group has 6 to 14 carbon atoms. The term “C₃₋₆ cycloalkyl” as used herein, means a saturated or unsaturated (but non-aromatic) cyclic hydrocarbon group containing from 3 to 6 carbons. The term “C₃₋₄ cycloalkyl” as used herein, means a saturated cyclic hydrocarbon group containing from 3 to 4 carbons.

Examples of C₃₋₄ cycloalkyl include cyclopropyl and cyclobutyl. Also included in the definition of cycloalkyl are moieties that have one or more aromatic rings (including aryl and heteroaryl) fused to the cycloalkyl ring, for example, benzo or pyridinyl derivatives of cyclopentane (a 5-membered cycloalkyl), cyclopentene, cyclohexane (a 6-membered cycloalkyl), and the like, for example, 6,7-dihydro-5H-cyclopenta[b]pyridinyl, 5,6,7,8-tetrahydroquinolinyl, or 1 5,6,7,8-tetrahydroisoquinolinyl, each of which includes a 5-membered or 6-membered cycloalkyl moiety that is fused to a heteroaryl ring (i.e. the pyridinyl ring). The cycloalkyl or C₃₋₄ cycloalkyl group optionally can be substituted by 1 or more (e.g., 1 to 5) suitable substituents when so specified.

The term “C₃₋₆ cycloalkyl-C₁₋₄ alkyl-” as used herein, means a C₃₋₆ cycloalkyl as defined herein, appended to the parent molecular moiety through a C₃₋₄ alkyl group, as defined herein. The term “C₃₋₄ cycloalkyl-C₁₋₄ alkyl-” as used herein, means a C₃₋₄ cycloalkyl as defined herein, appended to the parent molecular moiety through a C₃₋₄ alkyl group, as defined herein. Some examples of C₃₋₄ cycloalkyl-C₁₋₄ alkyl- include cyclopropylmethyl, 2-cyclopropylethyl, 2-cyclopropylpropyl, 3-cyclopropylpropyl, cyclobutylmethyl, 2-cyclobutylethyl, 2-cyclobutylpropyl, and 3-cyclobutylpropyl.

The term “C₃₋₆ cycloalkyl-C₁₋₂ alkyl-” as used herein, means a C₃₋₆ cycloalkyl as defined herein, appended to the parent molecular moiety through a C₁₋₂ alkyl group, as defined herein. The term “C₃₋₄ cycloalkyl-C₁₋₂ alkyl-” as used herein, means a C₃₋₄ cycloalkyl as defined herein, appended to the parent molecular moiety through a C₁₋₂ alkyl group, as defined herein.

As used herein, the term “heterocycloalkyl” refers to a monocyclic or polycyclic [including 2 or more rings that are fused together, including spiro, fused, or bridged systems, for example, a bicyclic ring system], saturated or unsaturated, non-aromatic 4- to 15-membered ring system (such as a 4- to 14-membered ring system, 4- to 12-membered ring system, 5- to 10-membered ring system, 4- to 7-membered ring system, 4- to 6-membered ring system, or 5- to 6-membered ring system), including 1 to 14 ring-forming carbon atoms and 1 to 10 ring-forming heteroatoms each independently selected from O, S and N (and optionally P or B when present). The heterocycloalkyl group can also optionally contain one or more oxo (i.e., ═O) or thiono (i.e., ═S) groups. For example, the term “4- to 7-membered heterocycloalkyl” refers to a monocyclic or polycyclic, saturated or unsaturated, non-aromatic 4- to 7-membered ring system that comprises one or more ring-forming heteroatoms each independently selected from O, S and N. For another example, the term “5- or 6-membered heterocycloalkyl” refers to a monocyclic or polycyclic, saturated or unsaturated, non-aromatic 5- or 6-membered ring system that comprises one or more ring-forming heteroatoms each independently selected from O, S and N. The heterocycloalkyl group optionally can be substituted by 1 or more (e.g., 1 to 5) suitable substituents, when so specified.

Some examples of 4- to 7-membered heterocycloalkyl include azetidinyl, oxetanyl, tetrahydrofuranyl, imidazolidinyl, pyrrolidinyl, piperidinyl, piperazinyl, oxazolidinyl, thiazolidinyl, pyrazolidinyl, thiomorpholinyl, tetrahydrothiazinyl, tetrahydrothiadiazinyl, morpholinyl, tetrahydrodiazinyl, and tetrahydropyranyl (also known as oxanyl). Some further examples of 4- to 7-heterocycloalkyl include tetrahydrofuran-2-yl, tetrahydrofuran-3-yl, tetrahydropyranyl (e.g., tetrahydro-2H-pyran-4-yl), imidazolidin-1-yl, imidazolidin-2-yl, imidazolidin-4-yl, pyrrolidin-1-yl, pyrrolidin-2-yl, pyrrolidin-3-yl, piperidin-1-yl, piperidin-2-yl, piperidin-3-yl, piperidin-4-yl, piperazin-1-yl, piperazin-2-yl, 1,3-oxazolidin-3-yl, 1,4-oxazepan-2-yl, isothiazolidinyl, 1,3-thiazolidin-3-yl, 1,2-pyrazolidin-2-yl, 1,2-tetrahydrothiazin-2-yl, 1,3-thiazinan-3-yl, 1,2-tetrahydrodiazin-2-yl, 1,3-tetrahydrodiazin-1-yl, 1,4-oxazin-4-yl, oxazolidinonyl, 2-oxo-piperidinyl (e.g., 2-oxo-piperidin-1-yl), 2-oxoazepan-3-yl, and the like.

As used herein, the term “heteroaryl” refers to monocyclic or fused-ring polycyclic aromatic heterocyclic groups with one or more heteroatom ring members (ring-forming atoms) each independently selected from O, S and N in at least one ring. The heteroaryl group has 5 to 14 ring-forming atoms, including 1 to 13 carbon atoms, and 1 to 8 heteroatoms selected from O, S, and N. In some embodiments, the heteroaryl group has 5 to 10 ring-forming atoms including one to four heteroatoms. The heteroaryl group can also contain one to three oxo or thiono (i.e., ═S) groups. In some embodiments, the heteroaryl group has 5 to 8 ring-forming atoms including one, two or three heteroatoms. For example, the term “5-membered heteroaryl” refers to a monocyclic heteroaryl group as defined above with 5 ring-forming atoms in the monocyclic heteroaryl ring; the term “6-membered heteroaryl” refers to a monocyclic heteroaryl group as defined above with 6 ring-forming atoms in the monocyclic heteroaryl ring; and the term “5- or 6-membered heteroaryl” refers to a monocyclic heteroaryl group as defined above with 5 or 6 ring-forming atoms in the monocyclic heteroaryl ring. A heteroaryl group optionally can be substituted by 1 or more (e.g., 1 to 5) suitable substituents, when so specified. Examples of monocyclic heteroaryls include those with 5 ring-forming atoms including one to three heteroatoms or those with 6 ring-forming atoms including one, two or three nitrogen heteroatoms. Examples of fused bicyclic heteroaryls include two fused 5- and/or 6-membered monocyclic rings including one to four heteroatoms.

Some examples of heteroaryl groups include pyridinyl (e.g., pyridin-2-yl, pyridin-3-yl, pyridine-4-yl), pyrazinyl, pyrimidinyl (e.g., pyrimidin-2-yl, pyrimidin-4-yl, or pyrimidin-5-yl), pyridazinyl (e.g., pyridazin-3-yl, or pyridazin-4-yl), thienyl, furyl, imidazolyl (e.g., 1H-imidazol-4-yl), pyrrolyl, oxazolyl (e.g., 1,3-oxazolyl, 1,2-oxazolyl), thiazolyl (e.g., 1,2-thiazolyl, 1,3-thiazolyl), pyrazolyl (e.g., pyrazol-1-yl, pyrazol-3-yl, pyrazol-4-yl), tetrazolyl (e.g., 2H-tetrazol-5-yl), triazolyl (e.g., 1,2,3-triazolyl, 1,2,4-triazolyl), oxadiazolyl (e.g., 1,2,3-oxadiazolyl, 1,2,4-oxadiazolyl or 1,3,4-oxadiazolyl), thiadiazolyl (e.g., 1,3,4-thiadiazolyl, or 1,2,4-thiadiazolyl), quinolyl, isoquinolyl, benzothienyl, benzofuryl, indolyl, benzothiazolyl, 1,2-benzoxazolyl, 1H-imidazo[4,5-c]pyridinyl, imidazo[1,2-a]pyridinyl, 1H-pyrrolo[3,2-c]pyridinyl, imidazo[1,2-a]pyrazinyl, imidazo[2,1-c][1,2,4]triazinyl, imidazo[1,5-a]pyrazinyl, imidazo[1,2-a]pyrimidinyl, 1H-indazolyl, 9H-purinyl, imidazo[1,2-a]pyrimidinyl, [1,2,4]triazolo[1,5-a]pyridinyl, [1,2,4]triazolo[1,5-a]pyrimidinyl, [1,2,4]triazolo[4,3-b]pyridazinyl, isoxazolo[5,4-c]pyridazinyl, isoxazolo[3,4-c]pyridazinyl, pyrazolo[1,5-a]pyrimidinyl, 6,7-dihydro-5H-pyrrolo[1,2-b][1,2,4]triazolyl, pyridone, pyrimidone, pyrazinone, pyrimidinone, 1H-imidazol-2(3H)-one, 1H-pyrrole-2,5-dione, 3-oxo-2H-pyridazinyl, 1H-2-oxo-pyrimidinyl, 1H-2-oxo-pyridinyl, 2,4(1H,3H)-dioxo-pyrimidinyl, 1H-2-oxo-pyrazinyl, and the like.

As used herein, the compound of Formula I as described herein includes optional substitutions and variables. It is understood that the normal valency of each of the designated (optionally substituted) atom or moiety is not exceeded, and that any of the optional substitution results in a stable compound. It is also understood that combinations of optional substituents and/or variables are permissible only if such combinations result in a stable compound.

As used herein, when a group is described to be optionally substituted, it means that the group can be either unsubstituted or substituted with one or more substitutents as specified.

As used herein, unless otherwise specified, the point of attachment of a substituent can be from any suitable position of the substituent. For example, piperidinyl can be piperidin-1-yl (attached through the N atom of the piperidinyl), piperidin-2-yl (attached through the C atom at the 2-position of the piperidinyl), piperidin-3-yl (attached through the C atom at the 3-position of the piperidinyl), or piperidin-4-yl (attached through the C atom at the 4-position of the piperidinyl). For another example, propanyl (or propyl) can be propan-1-yl (or 1-propyl) or propan-2-yl (or 2-propyl).

As used herein, the point of attachment of a substituent can be specified to indicate the position where the substituent is attached to another moiety. For example, “(C₃₋₄ cycloalkyl)-C₁₋₄ alkyl-” means the point of attachment occurs at the “C₁₋₄ alkyl” part of the “(C₃₋₄ cycloalkyl)-C₁₋₄ alkyl-.”

When a substituted or optionally substituted moiety is described without indicating the atom via which such moiety is bonded to a substituent, then the substituent may be bonded via any appropriate atom in such moiety. For example in a substituted “(C₃₋₄ cycloalkyl)-C₁₋₄ alkyl-”, a substituent on the cycloalkylalkyl [i.e., (C₃₋₄ cycloalkyl)-C₁₋₄ alkyl-] can be bonded to any carbon atom on the alkyl part or on the cycloalkyl part of the cycloalkylalkyl. Combinations of substituents and/or variables are permissible only if such combinations result in stable compounds.

As used herein, the term “adjacent” in describing the relative positions of two substituent groups on a ring structure refers to two substituent groups that are respectively attached to two ring-forming atoms of the same ring, wherein the two ring-forming atoms are directly connected through a chemical bond. For example, in the following structure:

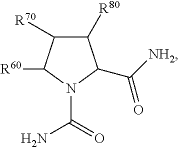

either of R⁶⁰ and R⁸⁰ is an adjacent group of R⁷⁰.

“Mammals” refers to warm-blooded vertebrate animals characterized by the secretion of milk by females for the nourishment of the young, such as guinea pigs, mice, rats, gerbils, cats, rabbits, dogs, cattle, goats, sheep, horses, monkeys, chimpanzees, and humans.

The term “pharmaceutically acceptable” means the substance (e.g., the compounds of the invention) and any salt thereof, or composition containing the substance or salt of the invention that is suitable for administration to a patient.

As used herein, the expressions “reaction-inert solvent” and “inert solvent” refer to a solvent or a mixture thereof which does not interact with starting materials, reagents, intermediates or products in a manner which adversely affects the yield of the desired product.

As used herein, the term “selectivity” or “selective” refers to a greater effect of a compound in a first assay, compared to the effect of the same compound in a second assay. For example, in “gut-selective” compounds, the first assay is for the half-life of the compound in the intestine and the second assay is for the half-life of the compound in the liver.

“Therapeutically effective amount” means an amount of a compound of the present invention that (i) treats or prevents the particular disease, condition, or disorder; (ii) attenuates, ameliorates, or eliminates one or more symptoms of the particular disease, condition, or disorder; or (iii) prevents or delays the onset of one or more symptoms of the particular disease, condition, or disorder described herein.

The term “treating”, “treat”, or “treatment” as used herein embraces both preventative, i.e., prophylactic, and palliative treatment, including reversing, relieving, alleviating, or slowing the progression of the disease (or disorder or condition) or any tissue damage associated with one or more symptoms of the disease (or disorder or condition).

As used herein, the term “contacting” refers to the bringing together of indicated moieties in an in vitro system or an in vivo system. For example, “contacting” GIPR with a compound of the invention includes the administration of a compound of the present invention to a mammal, such as a human, having the GIPR, as well as, for example, introducing a compound of the invention into a sample containing a cellular or purified preparation containing the GIPR.

Every embodiment, Example, or pharmaceutically acceptable salt thereof may be claimed individually or grouped together in any combination with any number of each and every embodiment described herein.

The compound of the invention [a compound of Formula I or a pharmaceutically acceptable salt thereof (including a compound of Formula Ia, II, IIa, III, IIIa, IV, IV-1, IV-2, IVa, IVa-1, IVa-2, V, Va, VI, VIa, VII, VIIa, or a pharmaceutically acceptble salt thereof)] can be used in any of the pharmaceutical compositions, uses, and methods of the invention described herein.

### Pharmaceutical Compositions

The present invention also provides a composition (e.g., a pharmaceutical composition) comprising the compound of the invention. Accordingly, in one embodiment, the invention provides a pharmaceutical composition comprising (a therapeutically effective amount of) the compound of the invention and optionally comprising a pharmaceutically acceptable carrier. In addition to the compounds of the invention, the pharmaceutical composition of the invention may also contain, or be co-administered (e.g. simultaneously, sequentially, together, or separately) with, one or more pharmacological agents of value in treating one or more disease conditions referred to herein. In one further embodiment, the invention provides a pharmaceutical composition comprising (a therapeutically effective amount of) a compound of Formula I or a pharmaceutically acceptable salt thereof, optionally comprising a pharmaceutically acceptable carrier and, optionally, at least one additional medicinal or pharmaceutical agent (such as an anti-diabetic agent or weight management agent). In one embodiment, the additional medicinal or pharmaceutical agent is anti-diabetic agent as described below.

A “pharmaceutical composition” of the invention refers to a mixture of (1) one or more of the compounds of the invention as an active ingredient (e.g. a compound of Formula I or a pharmaceutically acceptable salt, including any solvate, hydrate, solid form, stereoisomer, tautomer, or prodrug) and (2) at least one pharmaceutically acceptable excipient.

The term ‘excipient’ is used herein to describe any ingredient other than the compound(s) of the invention. The choice of excipient will to a large extent depend on factors such as the mode of administration, the effect of the excipient on solubility and stability, and the nature of the dosage form.

As used herein, “excipient” includes any and all solvents, dispersion media, coatings, antibacterial agents, antifungal agents, isotonic agents, absorption delaying agents, carriers, diluents and the like that are physiologically compatible. Examples of excipients include one or more of water, saline, phosphate buffered saline, dextrose, glycerol, ethanol and the like, as well as combinations thereof, and may include isotonic agents, for example, sugars, sodium chloride, or polyalcohols such as mannitol, or sorbitol in the composition. Examples of excipients also include various organic solvents (such as hydrates and solvates). The pharmaceutical compositions may, if desired, contain additional excipients such as flavorings, binders/binding agents, lubricating agents, disintegrants, sweetening or flavoring agents, coloring matters or dyes, and the like. For example, for oral administration, tablets containing various excipients, such as citric acid may be employed together with various disintegrants such as starch, alginic acid and certain complex silicates and with binding agents such as sucrose, gelatin and acacia. Examples, without limitation, of excipients include calcium carbonate, calcium phosphate, various sugars and types of starch, cellulose derivatives, gelatin, vegetable oils and polyethylene glycols. Additionally, lubricating agents such as magnesium stearate, sodium lauryl sulfate and talc are often useful for tableting purposes. Solid compositions of a similar type may also be employed in soft and hard filled gelatin capsules. Non-limiting examples of excipients, therefore, also include lactose or milk sugar and high molecular weight polyethylene glycols. When aqueous suspensions or elixirs are desired for oral administration the active compound therein may be combined with various sweetening or flavoring agents, coloring matters or dyes and, if desired, emulsifying agents or suspending agents, together with additional excipients such as water, ethanol, propylene glycol, glycerin, or combinations thereof.

Examples of excipients also include pharmaceutically acceptable substances such as wetting agents or minor amounts of auxiliary substances such as wetting or emulsifying agents, preservatives, or buffers, which enhance the shelf life or effectiveness of the compound.

The compositions of this invention may be in a variety of forms. These include, for example, liquid, semi-solid and solid dosage forms, such as liquid solutions (e.g., injectable and infusible solutions), dispersions or suspensions, tablets, capsules, pills, powders, liposomes and suppositories. The form depends on the intended mode of administration and therapeutic application.

Some compositions are in the form of injectable or infusible solutions, such as compositions similar to those used for passive immunization of humans with antibodies in general. One mode of administration is parenteral (e.g., intravenous, subcutaneous, intraperitoneal, intramuscular). In another embodiment, the compound is administered by intravenous infusion or injection. In yet another embodiment, the compound is administered by intramuscular or subcutaneous injection.

Oral administration of a solid dosage form may be, for example, presented in discrete units, such as hard or soft capsules, pills, cachets, lozenges, or tablets, each containing a predetermined amount of at least one compound of the invention. In another embodiment, the oral administration may be in a powder or granule form. In another embodiment, the oral dosage form is sub-lingual, such as, for example, a lozenge. In such solid dosage forms, the compounds of the invention are ordinarily combined with one or more adjuvants. Such capsules or tablets may comprise a controlled release formulation. In the case of capsules, tablets, and pills, the dosage forms also may comprise buffering agents or may be prepared with enteric coatings.

In another embodiment, oral administration may be in a liquid dosage form. Liquid dosage forms for oral administration include, for example, pharmaceutically acceptable emulsions, solutions, suspensions, syrups, and elixirs containing inert diluents commonly used in the art (e.g., water). Such compositions also may comprise adjuvants, such as one or more of wetting, emulsifying, suspending, flavoring (e.g., sweetening), or perfuming agents.

In another embodiment, the invention comprises a parenteral dosage form. “Parenteral administration” includes, for example, subcutaneous injections, intravenous injections, intraperitoneal injections, intramuscular injections, intrasternal injections, and infusion. Injectable preparations (i.e., sterile injectable aqueous or oleaginous suspensions) may be formulated according to the known art using one or more of suitable dispersing, wetting agents, or suspending agents.

In another embodiment, the invention comprises a topical dosage form. “Topical administration” includes, for example, dermal and transdermal administration, such as via transdermal patches or iontophoresis devices, intraocular administration, or intranasal or inhalation administration. Compositions for topical administration also include, for example, topical gels, sprays, ointments, and creams. A topical formulation may include a compound which enhances absorption or penetration of the active ingredient through the skin or other affected areas. When the compounds of this invention are administered by a transdermal device, administration will be accomplished using a patch either of the reservoir and porous membrane type or of a solid matrix variety. Typical formulations for this purpose include gels, hydrogels, lotions, solutions, creams, ointments, dusting powders, dressings, foams, films, skin patches, wafers, implants, sponges, fibers, bandages and microemulsions. Liposomes may also be used. Typical excipients include alcohol, water, mineral oil, liquid petrolatum, white petrolatum, glycerin, polyethylene glycol and propylene glycol. Penetration enhancers may be incorporated—see, for example, B. C. Finnin and T. M. Morgan, J. Pharm. Sci., vol. 88, pp. 955-958, 1999.

Formulations suitable for topical administration to the eye include, for example, eye drops wherein the compound of this invention is dissolved or suspended in a suitable excipient. A typical formulation suitable for ocular or aural administration may be in the form of drops of a micronized suspension or solution in isotonic, pH-adjusted, sterile saline. Other formulations suitable for ocular and aural administration include ointments, biodegradable (i.e., absorbable gel sponges, collagen) and non-biodegradable (i.e., silicone) implants, wafers, lenses and particulate or vesicular systems, such as niosomes or liposomes. A polymer such as crossed linked polyacrylic acid, polyvinyl alcohol, hyaluronic acid, a cellulosic polymer, for example, hydroxypropylmethylcellulose, hydroxyethylcellulose, or methylcellulose, or a heteropolysaccharide polymer, for example, gelan gum, may be incorporated together with a preservative, such as benzalkonium chloride. Such formulations may also be delivered by iontophoresis.

For intranasal administration, the compounds of the invention are conveniently delivered in the form of a solution or suspension from a pump spray container that is squeezed or pumped by the patient or as an aerosol spray presentation from a pressurized container or a nebulizer, with the use of a suitable propellant. Formulations suitable for intranasal administration are typically administered in the form of a dry powder (either alone, as a mixture, for example, in a dry blend with lactose, or as a mixed component particle, for example, mixed with phospholipids, such as phosphatidylcholine) from a dry powder inhaler or as an aerosol spray from a pressurized container, pump, spray, atomizer (preferably an atomizer using electrohydrodynamics to produce a fine mist), or nebulizer, with or without the use of a suitable propellant, such as 1,1,1,2-tetrafluoroethane or 1,1,1,2,3,3,3-heptafluoropropane. For intranasal use, the powder may comprise a bioadhesive agent, for example, chitosan or cyclodextrin.

In another embodiment, the invention comprises a rectal dosage form. Such rectal dosage form may be in the form of, for example, a suppository. Cocoa butter is a traditional suppository base, but various alternatives may be used as appropriate.

Other excipients and modes of administration known in the pharmaceutical art may also be used. Pharmaceutical compositions of the invention may be prepared by any of the well-known techniques of pharmacy, such as effective formulation and administration procedures. The above considerations in regard to effective formulations and administration procedures are well known in the art and are described in standard textbooks. Formulation of drugs is discussed in, for example, Ansel, Howard C., et al., Ansel's Pharmaceutical Dosage Forms and Drug Delivery Systems. Philadelphia: Lippincott, Williams & Wilkins, 2004; Gennaro, Alfonso R., et al. Remington: The Science and Practice of Pharmacy. Philadelphia: Lippincott, Williams & Wilkins, 2000; Rowe, Raymond C. Handbook of Pharmaceutical Excipients. Chicago, Pharmaceutical Press, 2005; Stahl, P. Heinrich and Camilli G. Wermuth, Eds. Handbook of Pharmaceutical Salts: Properties, Selection, and Use. New York: Wiley-VCH, 2011; and Brittain, Harry G., Ed. Polymorphism in Pharmaceutical Solids. New York: Informa Healthcare USA, Inc., 2016.

Acceptable excipients are nontoxic to subjects at the dosages and concentrations employed, and may comprise one or more of the following: 1) buffers such as phosphate, citrate, or other organic acids; 2) salts such as sodium chloride; 3) antioxidants such as ascorbic acid or methionine; 4) preservatives such as octadecyldimethylbenzyl ammonium chloride, hexamethonium chloride, benzalkonium chloride, benzethonium chloride, phenol, butyl or benzyl alcohol; 5) alkyl parabens such as methyl or propyl paraben, catechol, resorcinol, cyclohexanol, 3-pentanol, or m-cresol; 6) low molecular weight (less than about 10 residues) polypeptides; 7) proteins such as serum albumin, gelatin, or immunoglobulins; 8) hydrophilic polymers such as polyvinylpyrrolidone; 9) amino acids such as glycine, glutamine, asparagine, histidine, arginine, or lysine; 10) monosaccharides, disaccharides, or other carbohydrates including glucose, mannose, or dextrins; 11) chelating agents such as EDTA; 12) sugars such as sucrose, mannitol, trehalose or sorbitol; 13) salt-forming counter-ions such as sodium, metal complexes (e.g., Zn-protein complexes), or 14) non-ionic surfactants such as polysorbates (e.g., polysorbate 20 or polysorbate 80), poloxamers or polyethylene glycol (PEG).

For oral administration, the compositions may be provided in the form of tablets or capsules containing 0.01, 0.05, 0.1, 0.5, 1.0, 2.5, 5.0, 10.0, 15.0, 25.0, 50.0, 75.0, 100, 125, 150, 175, 200, 250 or 500 milligrams of the active ingredient for the symptomatic adjustment of the dosage to the patient. A medicament typically contains from about 0.01 mg to about 500 mg of the active ingredient, or in another embodiment, from about 1 mg to about 100 mg of active ingredient. Intravenously, doses may range from about 0.01 to about 10 mg/kg/minute during a constant rate infusion.

Liposome-containing compounds of the invention may be prepared by methods known in the art (See, for example, Chang, H. I.; Yeh, M. K.; Clinical development of liposome-based drugs: formulation, characterization, and therapeutic efficacy; Int J Nanomedicine 2012; 7; 49-60). Particularly useful liposomes may be generated by the reverse phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter.

Compounds of the invention may also be entrapped in microcapsules prepared, for example, by coacervation techniques or by interfacial polymerization, for example, hydroxymethylcellulose or gelatin microcapsules and poly-(methylmethacrylate) microcapsules, respectively, in colloidal drug delivery systems (for example, liposomes, albumin microspheres, microemulsions, nano-particles and nanocapsules) or in macroemulsions. Such techniques are disclosed in Remington, The Science and Practice of Pharmacy, 20th Ed., Mack Publishing (2000).

Sustained-release preparations may be used. Suitable examples of sustained-release preparations include semi-permeable matrices of solid hydrophobic polymers containing a compound of the invention, which matrices are in the form of shaped articles, e.g., films, or microcapsules. Examples of sustained-release matrices include polyesters, hydrogels (for example, poly(2-hydroxyethyl-methacrylate), or ‘poly(vinylalcohol)), polylactides, copolymers of L-glutamic acid and 7 ethyl-L-glutamate, non-degradable ethylene-vinyl acetate, degradable lactic acid-glycolic acid copolymers such as those used in leuprolide acetate for depot suspension (injectable microspheres composed of lactic acid-glycolic acid copolymer and leuprolide acetate), sucrose acetate isobutyrate, and poly-D-(−)-3-hydroxybutyric acid.

The formulations to be used for intravenous administration must be sterile. This is readily accomplished by, for example, filtration through sterile filtration membranes. Compounds of the invention are generally placed into a container having a sterile access port, for example, an intravenous solution bag or vial having a stopper pierceable by a hypodermic injection needle.

Suitable emulsions may be prepared using commercially available fat emulsions, such as a lipid emulsions comprising soybean oil, a fat emulsion for intravenous administration (e.g., comprising safflower oil, soybean oil, egg phosphatides and glycerin in water), emulsions containing soya bean oil and medium-chain triglycerides, and lipid emulsions of cottonseed oil. The active ingredient may be either dissolved in a pre-mixed emulsion composition or alternatively it may be dissolved in an oil (e.g., soybean oil, safflower oil, cottonseed oil, sesame oil, corn oil or almond oil) and an emulsion formed upon mixing with a phospholipid (e.g., egg phospholipids, soybean phospholipids or soybean lecithin) and water. It will be appreciated that other ingredients may be added, for example glycerol or glucose, to adjust the tonicity of the emulsion. Suitable emulsions will typically contain up to 20% oil, for example, between 5 and 20%. The fat emulsion may comprise fat droplets between 0.1 and 1.0 μm, particularly 0.1 and 0.5 μm, and have a pH in the range of 5.5 to 8.0.

For example, the emulsion compositions may be those prepared by mixing a compound of the invention with a lipid emulsions comprising soybean oil or the components thereof (soybean oil, egg phospholipids, glycerol and water).

Compositions for inhalation or insufflation include solutions and suspensions in pharmaceutically acceptable aqueous or organic solvents, or mixtures thereof, and powders. The liquid or solid compositions may contain suitable pharmaceutically acceptable excipients as set out above. In some embodiments, the compositions are administered by the oral or nasal respiratory route for local or systemic effect. Compositions in preferably sterile pharmaceutically acceptable solvents may be nebulized by use of gases. Nebulized solutions may be breathed directly from the nebulizing device or the nebulizing device may be attached to a face mask, tent or intermittent positive pressure breathing machine. Solution, suspension or powder compositions may be administered, preferably orally or nasally, from devices which deliver the formulation in an appropriate manner.

A drug product intermediate (DPI) is a partly processed material that must undergo further processing steps before it becomes bulk drug product. Compounds of the invention may be formulated into drug product intermediate DPI containing the active ingredient in a higher free energy form than the crystalline form. One reason to use a DPI is to improve oral absorption characteristics due to low solubility, slow dissolution, improved mass transport through the mucus layer adjacent to the epithelial cells, and in some cases, limitations due to biological barriers such as metabolism and transporters. Other reasons may include improved solid state stability and downstream manufacturability. In one embodiment, the drug product intermediate contains a compound of the invention isolated and stabilized in the amorphous state (for example, amorphous solid dispersions (ASDs)). There are many techniques known in the art to manufacture ASD's that produce material suitable for integration into a bulk drug product, for example, spray dried dispersions (SDD's), melt extrudates (often referred to as HME's), co-precipitates, amorphous drug nanoparticles, and nano-adsorbates. In one embodiment amorphous solid dispersions comprise a compound of the invention and a polymer excipient. Other excipients as well as concentrations of said excipients and the compound of the invention are well known in the art and are described in standard textbooks. See, for example, “Amorphous Solid Dispersions Theory and Practice” by Navnit Shah et al.

The pharmaceutical composition may, for example, be in a form suitable for oral administration as a tablet, capsule, pill, powder, sustained release formulation, solution or suspension, for parenteral injection as a sterile solution, suspension or emulsion, for topical administration as an ointment or cream or for rectal administration as a suppository.

Exemplary parenteral administration forms include solutions or suspensions of active compounds in sterile aqueous solutions, for example, aqueous propylene glycol or dextrose solutions. Such dosage forms may be suitably buffered, if desired.

The pharmaceutical composition may be in unit dosage forms suitable for single administration of precise dosages. One of ordinary skill in the art would appreciate that the composition may be formulated in sub-therapeutic dosage such that multiple doses are envisioned.

In one embodiment the composition comprises (a therapeutically effective amount of) a compound of Formula I or a pharmaceutically acceptable salt thereof and a pharmaceutically acceptable excipient.

### Administration and Dosing

The term “treating”, “treat” or “treatment” as used herein embraces both preventative, i.e., prophylactic, and palliative treatment, i.e., relieve, alleviate, or slow the progression of the patient's disease (or condition) or any tissue damage associated with the disease.

As used herein, the terms, “subject, “individual” or “patient,” used interchangeably, refer to any animal, including mammals. Mammals according to the invention include canine, feline, bovine, caprine, equine, ovine, porcine, rodents, lagomorphs, primates, humans and the like, and encompass mammals in utero. In an embodiment, humans are suitable subjects. Human subjects may be of any gender and at any stage of development.

As used herein, the phrase “therapeutically effective amount” refers to the amount of active compound or pharmaceutical agent that elicits the biological or medicinal response in a tissue, system, animal, individual or human that is being sought by a researcher, veterinarian, medical doctor or other clinician, which may include one or more of the following:
(1) preventing a condition, disease, or disorder; for example, preventing the condition, disease, or disorder in an individual that may be predisposed to the condition, disease, or disorder but does not yet experience or display the pathology or symptomatology of the disease;
(2) inhibiting a condition, disease, or disorder; for example, inhibiting the condition, disease, or disorder in an individual that is experiencing or displaying the pathology or symptomatology of the condition, disease, or disorder [i.e., arresting (or slowing) further development of the pathology or symptomatology or both]; and
(3) ameliorating a condition, disease, or disorder; for example, ameliorating the condition, disease, or disorder in an individual that is experiencing or displaying the pathology or symptomatology of the condition, disease, or disorder [i.e., reversing the pathology or symptomatology or both].

Typically, a compound of the invention is administered in an amount effective to treat a condition, disease, or disorder as described herein. The compounds of the invention may be administered as compound in the free form, or alternatively, as a pharmaceutically acceptable salt. For administration and dosing purposes, the compound in free form or pharmaceutically acceptable salt thereof will simply be referred to as the compounds of the invention.

The compounds of the invention are administered by any suitable route in the form of a pharmaceutical composition adapted to such a route, and in a dose effective for the treatment intended. Administration of the compounds of this invention can be via any method which delivers a compound of this invention systemically and/or locally. The compounds of the invention may be administered orally, rectally, vaginally, parenterally (including, e.g., intravenous, subcutaneous, intramuscular, intravascular or infusion), topically, intranasally, or by inhalation.

The compounds of the invention may be administered orally. Oral administration may involve swallowing, so that the compound enters the gastrointestinal tract, or buccal or sublingual administration may be employed by which the compound enters the bloodstream directly from the mouth.

In another embodiment, the compounds of the invention may also be administered parenterally, for example directly into the bloodstream, into muscle, or into an internal organ. Suitable means for parenteral administration include intravenous, intraarterial, intraperitoneal, intrathecal, intraventricular, intraurethral, intrasternal, intracranial, intramuscular and subcutaneous. Suitable devices for parenteral administration include needle (including microneedle) injectors, needle-free injectors, and infusion techniques.

In another embodiment, the compounds of the invention may also be administered topically to the skin or mucosa, that is, dermally or transdermally. In another embodiment, the compounds of the invention may also be administered intranasally or by inhalation. In another embodiment, the compounds of the invention may be administered rectally or vaginally. In another embodiment, the compounds of the invention may also be administered directly to the eye or ear.

The dosage regimen for the compounds of the invention or compositions containing said compounds is based on a variety of factors, including the type, age, weight, sex and medical condition of the patient; the severity of the condition; the route of administration; and the activity of the particular compound employed. Thus, the dosage regimen may vary widely. In one embodiment, the total daily dose of a compound of the invention is typically from about 0.0001 to about 100 mg/kg (i.e., mg compound of the invention per kg body weight) for the treatment of the indicated conditions discussed herein. In another embodiment, total daily dose of the compound of the invention is from about 0.01 to about 50 mg/kg; and in another embodiment, from about 0.1 to about 50 mg/kg; and in another embodiment, from about 0.5 to about 30 mg/kg. It is not uncommon that the administration of the compounds of the invention will be repeated a plurality of times in a day (typically no greater than 4 times). Multiple doses per day typically may be used to increase the total daily dose, if desired.

### Methods and Uses

Another embodiment of the present invention includes a compound of Formula I or a pharmaceutically acceptable salt of the compound for use as a medicament, particularly wherein the medicament is for use in the treatment or prevention of a GIPR-related condition, disease, or disorder, including administering to a mammal, such as a human, in need of such treatment.

Another embodiment of the present invention includes use of a compound of Formula I or a pharmaceutically acceptable salt of the compound as a medicament, particularly wherein the medicament is for use in the treatment or prevention of a GIPR-related condition, disease, or disorder, including administering to a mammal, such as a human, in need of such treatment.

Another embodiment of the present invention includes use of a compound of Formula I or a pharmaceutically acceptable salt of the compound in the manufacture of a medicament for treating or preventing a GIPR-related condition, disease, or disorder, including administering to a mammal, such as a human, in need of such treatment a therapeutically effective amount.

Another embodiment of the present invention includes the compound of invention for use as a medicament, particularly wherein the medicament is for use in treating or preventing a condition, disease, or disorder selected from diabetes [e.g. Type 1 diabetes mellitus (T1D), Type 2 diabetes mellitus (T2DM), including pre-diabetes], idiopathic T1D (Type 1b), latent autoimmune diabetes in adults (LADA), early-onset T2DM (EOD), youth-onset atypical diabetes (YOAD), maturity onset diabetes of the young (MODY), malnutrition-related diabetes, gestational diabetes, hyperglycemia, insulin resistance, hepatic insulin resistance, impaired glucose tolerance, diabetic neuropathy, diabetic nephropathy, kidney disease [e.g., acute kidney disorder, tubular dysfunction, proinflammatory changes to the proximal tubules, or chronic kidney disease (CKD)], diabetic retinopathy, adipocyte dysfunction, visceral adipose deposition, sleep apnea [e.g. obstructive sleep apnea (OSA)], obesity (including hypothalamic obesity and monogenic obesity) and related comorbidities (e.g., osteoarthritis and urine incontinence), eating disorders (including binge eating syndrome, bulimia nervosa, and syndromic obesity such as Prader-Willi and Bardet-Biedl syndromes), weight gain such as weight gain caused by use of other agents (e.g., caused by use of steroids and/or antipsychotics, or caused by treatment of depression, or caused by use of agents on cognitive function), excessive sugar craving, dyslipidemia [including hyperlipidemia, hypertriglyceridemia, increased total cholesterol, high LDL (low-density lipoprotein) cholesterol, and low HDL (high-density lipoprotein) cholesterol], hyperinsulinemia, nonalcoholic fatty liver disease [NAFLD, including related diseases such as steatosis, nonalcoholic steatohepatitis (NASH), fibrosis, cirrhosis, and hepatocellular carcinoma], cardiovascular disease, atherosclerosis (including coronary artery disease), peripheral vascular disease, hypertension, endothelial dysfunction, impaired vascular compliance, heart failure [e.g. congestive heart failure, heart failure with preserved ejection fraction (HFpEF), heart failure with reduced ejection fraction (HFrEF)], myocardial infarction (e.g. necrosis and apoptosis), stroke, hemorrhagic stroke, ischemic stroke, traumatic brain injury, pulmonary hypertension, restenosis after angioplasty, intermittent claudication, post-prandial lipemia, metabolic acidosis, ketosis, arthritis, osteoporosis, osteoarthritis, Parkinson's disease, left ventricular hypertrophy, peripheral arterial disease (PAD), macular degeneration, cataract, glomerulosclerosis, chronic renal failure, metabolic syndrome, syndrome X, premenstrual syndrome, angina pectoris, thrombosis, atherosclerosis, transient ischemic attacks, vascular restenosis, impaired glucose metabolism, conditions of impaired fasting plasma glucose, hyperuricemia, gout, erectile dysfunction, skin and connective tissue disorders, psoriasis, foot ulcerations, ulcerative colitis, hyper apo B lipoproteinemia, Alzheimer's Disease, schizophrenia, impaired cognition, inflammatory bowel disease, short bowel syndrome, Crohn's disease, colitis, irritable bowel syndrome, polycystic ovary syndrome (PCOS), and addiction (e.g., addition to alcohol, nicotine, and/or drug).

Another embodiment of the present invention includes use of the compound of invention as a medicament, particularly wherein the medicament is for use in the treatment or prevention of a condition, disease, or disorder selected from diabetes [e.g. Type 1 diabetes mellitus (T1D), Type 2 diabetes mellitus (T2DM), including pre-diabetes], idiopathic T1D (Type 1b), latent autoimmune diabetes in adults (LADA), early-onset T2DM (EOD), youth-onset atypical diabetes (YOAD), maturity onset diabetes of the young (MODY), malnutrition-related diabetes, gestational diabetes, hyperglycemia, insulin resistance, hepatic insulin resistance, impaired glucose tolerance, diabetic neuropathy, diabetic nephropathy, kidney disease [e.g., acute kidney disorder, tubular dysfunction, proinflammatory changes to the proximal tubules, or chronic kidney disease (CKD)], diabetic retinopathy, adipocyte dysfunction, visceral adipose deposition, sleep apnea [e.g. obstructive sleep apnea (OSA)], obesity (including hypothalamic obesity and monogenic obesity) and related comorbidities (e.g., osteoarthritis and urine incontinence), eating disorders (including binge eating syndrome, bulimia nervosa, and syndromic obesity such as Prader-Willi and Bardet-Biedl syndromes), weight gain such as weight gain caused by use of other agents (e.g., caused by use of steroids and/or antipsychotics, or caused by treatment of depression, or caused by use of agents on cognitive function), excessive sugar craving, dyslipidemia [including hyperlipidemia, hypertriglyceridemia, increased total cholesterol, high LDL (low-density lipoprotein) cholesterol, and low HDL (high-density lipoprotein) cholesterol], hyperinsulinemia, nonalcoholic fatty liver disease [NAFLD, including related diseases such as steatosis, nonalcoholic steatohepatitis (NASH), fibrosis, cirrhosis, and hepatocellular carcinoma], cardiovascular disease, atherosclerosis (including coronary artery disease), peripheral vascular disease, hypertension, endothelial dysfunction, impaired vascular compliance, heart failure [e.g. congestive heart failure, heart failure with preserved ejection fraction (HFpEF), heart failure with reduced ejection fraction (HFrEF)], myocardial infarction (e.g. necrosis and apoptosis), stroke, hemorrhagic stroke, ischemic stroke, traumatic brain injury, pulmonary hypertension, restenosis after angioplasty, intermittent claudication, post-prandial lipemia, metabolic acidosis, ketosis, arthritis, osteoporosis, osteoarthritis, Parkinson's disease, left ventricular hypertrophy, peripheral arterial disease (PAD), macular degeneration, cataract, glomerulosclerosis, chronic renal failure, metabolic syndrome, syndrome X, premenstrual syndrome, angina pectoris, thrombosis, atherosclerosis, transient ischemic attacks, vascular restenosis, impaired glucose metabolism, conditions of impaired fasting plasma glucose, hyperuricemia, gout, erectile dysfunction, skin and connective tissue disorders, psoriasis, foot ulcerations, ulcerative colitis, hyper apo B lipoproteinemia, Alzheimer's Disease, schizophrenia, impaired cognition, inflammatory bowel disease, short bowel syndrome, Crohn's disease, colitis, irritable bowel syndrome, polycystic ovary syndrome (PCOS), and addiction (e.g., addition to alcohol, nicotine, and/or drug).

Another embodiment of the present invention includes use of the compound of invention for the manufacture of a medicament for treating or preventing a condition, disease, or disorder selected from diabetes [e.g. Type 1 diabetes mellitus (T1 D), Type 2 diabetes mellitus (T2DM), including pre-diabetes], idiopathic T1D (Type 1b), latent autoimmune diabetes in adults (LADA), early-onset T2DM (EOD), youth-onset atypical diabetes (YOAD), maturity onset diabetes of the young (MODY), malnutrition-related diabetes, gestational diabetes, hyperglycemia, insulin resistance, hepatic insulin resistance, impaired glucose tolerance, diabetic neuropathy, diabetic nephropathy, kidney disease [e.g., acute kidney disorder, tubular dysfunction, proinflammatory changes to the proximal tubules, or chronic kidney disease (CKD)], diabetic retinopathy, adipocyte dysfunction, visceral adipose deposition, sleep apnea [e.g. obstructive sleep apnea (OSA)], obesity (including hypothalamic obesity and monogenic obesity) and related comorbidities (e.g., osteoarthritis and urine incontinence), eating disorders (including binge eating syndrome, bulimia nervosa, and syndromic obesity such as Prader-Willi and Bardet-Biedl syndromes), weight gain such as weight gain caused by use of other agents (e.g., caused by use of steroids and/or antipsychotics, or caused by treatment of depression, or caused by use of agents on cognitive function), excessive sugar craving, dyslipidemia [including hyperlipidemia, hypertriglyceridemia, increased total cholesterol, high LDL (low-density lipoprotein) cholesterol, and low HDL (high-density lipoprotein) cholesterol], hyperinsulinemia, nonalcoholic fatty liver disease [NAFLD, including related diseases such as steatosis, nonalcoholic steatohepatitis (NASH), fibrosis, cirrhosis, and hepatocellular carcinoma], cardiovascular disease, atherosclerosis (including coronary artery disease), peripheral vascular disease, hypertension, endothelial dysfunction, impaired vascular compliance, heart failure [e.g. congestive heart failure, heart failure with preserved ejection fraction (HFpEF), heart failure with reduced ejection fraction (HFrEF)], myocardial infarction (e.g. necrosis and apoptosis), stroke, hemorrhagic stroke, ischemic stroke, traumatic brain injury, pulmonary hypertension, restenosis after angioplasty, intermittent claudication, post-prandial lipemia, metabolic acidosis, ketosis, arthritis, osteoporosis, osteoarthritis, Parkinson's disease, left ventricular hypertrophy, peripheral arterial disease (PAD), macular degeneration, cataract, glomerulosclerosis, chronic renal failure, metabolic syndrome, syndrome X, premenstrual syndrome, angina pectoris, thrombosis, atherosclerosis, transient ischemic attacks, vascular restenosis, impaired glucose metabolism, conditions of impaired fasting plasma glucose, hyperuricemia, gout, erectile dysfunction, skin and connective tissue disorders, psoriasis, foot ulcerations, ulcerative colitis, hyper apo B lipoproteinemia, Alzheimer's Disease, schizophrenia, impaired cognition, inflammatory bowel disease, short bowel syndrome, Crohn's disease, colitis, irritable bowel syndrome, polycystic ovary syndrome (PCOS), and addiction (e.g., addition to alcohol, nicotine, and/or drug).

In some further embodiments of the methods and uses of the present invention described herein, the condition, disease, or disorder that can be treated or prevented in accordance with the present invention is selected from obesity, T2DM, Heart Failure (e.g. HFpEF and HFrEF); CKD; NAFLD, NASH, atherosclerosis, PAD, obstructive sleep apnea, diabetic retinopathy, and diabetic neuropathy.

The compound of the invention is a GIPR antagonist. Thus, the present invention further provides a method for modulating (e.g. antagonizing) GIPR (either in vitro or in vivo), comprising contacting (including incubating) the GIPR with the compound of Formula I or a pharmaceutically acceptable salt thereof (such as one selected from Examples 1-229 herein) described herein.

In some embodiments, the amount of the compound of the invention used in any one of the methods (or uses) of the present invention is effective in antagonizing GIPR.

### Stereoisomers

The compounds of the present invention may contain asymmetric or chiral centers, and, therefore, exist in two or more stereoisomeric forms. Unless specified otherwise, it is intended that all stereoisomeric forms of the compounds of the present invention as well as mixtures thereof, including racemic mixtures, form part of the present invention. In addition, the present invention embraces all geometric and positional isomers. For example, if a compound of the present invention incorporates a double bond or a fused ring, both the cis- and trans- forms, as well as mixtures, are embraced within the scope of the invention.

Stereoisomers of the compounds may include cis and trans isomers (geometric isomers), optical isomers such as R and S enantiomers, diastereomers, rotational isomers, atropisomers, and conformational isomers. For example, compounds of the invention containing one or more asymmetric carbon atoms may exist as two or more stereoisomers. Where a compound of the invention contains an alkenyl or alkenylene group, geometric cis/trans (or Z/E) isomers are possible. Cis/trans isomers may also exist for saturated rings.

The pharmaceutically acceptable salts of compounds of the invention may also contain a counterion which is optically active (e.g., D-lactate or L-lysine) or racemic (e.g. DL-tartrate or DL-arginine).

Cis/trans isomers may be separated by conventional techniques well known to those skilled in the art, for example, chromatography and fractional crystallization.

Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC). Alternatively, the racemate (or a racemic precursor) may be reacted with a suitable optically active compound, for example, an alcohol, or, in the case where a compound of the invention contains an acidic or basic moiety, a base or acid such as 1-phenylethylamine or tartaric acid. The resulting diastereomeric mixture may be separated by chromatography, fractional crystallization, or by using both of said techniques, and one or both of the diastereoisomers converted to the corresponding pure enantiomer(s) by means well known to a skilled person. Chiral compounds of the invention (and chiral precursors thereof) may be obtained in enantiomerically-enriched form using chromatography, typically HPLC. Concentration of the eluate affords the enriched mixture. Chiral chromatography using sub- and supercritical fluids may be employed. Methods for chiral chromatography useful in some embodiments of the present invention are known in the art (see, for example, Smith, Roger M., Loughborough University, Loughborough, UK; Chromatographic Science Series (1998), 75 (Supercritical Fluid Chromatography with Packed Columns), pp. 223-249 and references cited therein).

When any racemate crystallizes, crystals of two different types are possible. The first type is the racemic compound (true racemate) referred to above wherein one homogeneous form of crystal is produced containing both enantiomers in equimolar amounts. The second type is the racemic mixture or conglomerate wherein two crystal forms are produced in equimolar amounts each comprising a single enantiomer. While both of the crystal forms present in a racemic mixture have identical physical properties, they may have different physical properties compared to the true racemate. Racemic mixtures may be separated by conventional techniques known to those skilled in the art—see, for example, Stereochemistry of Organic Compounds by E. L. Eliel and S. H. Wilen (Wiley, 1994).

Chiral compounds of the invention (and chiral precursors thereof) may be obtained in enantiomerically-enriched form using chromatography, typically high pressure liquid chromatography (HPLC) or supercritical fluid chromatography (SFC), on a resin with an asymmetric stationary phase and with a mobile phase consisting of a hydrocarbon, typically heptane or hexane, containing from 0 to 50% isopropanol, typically from 2 to 20%, and from 0 to 5% of an alkylamine, typically 0.1% diethylamine (DEA) or isopropylamine. Concentration of the eluent affords the enriched mixture. In the case where SFC is used, the mobile phase may consist of a supercritical fluid, typically carbon dioxide, containing 2-50% of an alcohol, such as methanol, ethanol or isopropanol.

Diastereomeric mixtures can be separated into their individual diastereoisomers on the basis of their physicochemical differences by methods well known to those skilled in the art, such as by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereoisomers and converting (e.g., hydrolyzing) the individual diastereoisomers to the corresponding pure enantiomers. Enantiomers can also be separated by use of a chiral HPLC column. Alternatively, the specific stereoisomers may be synthesized by using an optically active starting material, by asymmetric synthesis using optically active reagents, substrates, catalysts or solvents, or by converting one stereoisomer into the other by asymmetric transformation.

In some embodiments, the compounds of the invention may have asymmetric carbon atoms. The carbon-carbon bonds of the compounds of Formula I may be depicted herein using a [symbol] solid line ( [symbol] ), a wavy line ( [symbol] ), a solid wedge ( [symbol] ), or a dotted wedge ( [symbol] ).

The use of a solid line to depict bonds to asymmetric carbon atoms is meant to indicate that all possible stereoisomers (e.g., specific enantiomers, racemic mixtures, etc.) at that carbon atom are included. The use of either a solid or dotted wedge to depict bonds to asymmetric carbon atoms is meant to indicate that only the stereoisomer shown is meant to be included. The use of a wavy line to depict bonds to asymmetric carbon atoms is meant to indicate that the stereochemistry is unknown (unless otherwise specified). It is possible that compounds of the invention may contain more than one asymmetric carbon atom. In those compounds, the use of a solid line to depict bonds to asymmetric carbon atoms is meant to indicate that all possible stereoisomers are meant to be included. For example, unless stated otherwise, it is intended that the compounds of the invention can exist as enantiomers and diastereomers or as racemates and mixtures thereof. The use of a solid line to depict bonds to one or more asymmetric carbon atoms in a compound of the invention and the use of a solid or dotted wedge to depict bonds to other asymmetric carbon atoms in the same compound is meant to indicate that a mixture of diastereomers is present.

Where the compounds of the present invention possess two or more stereogenic centers and the absolute or relative stereochemistry is given in the name, the designations R and S refer respectively to each stereogenic center in ascending numerical order (1, 2, 3, etc.) according to the conventional IUPAC number schemes for each molecule. Where the compounds of the present invention possess one or more stereogenic centers and no stereochemistry is given in the name or structure, it is understood that the name or structure is intended to encompass all forms of the compound, including the racemic form.

The compounds of this invention may contain olefin-like double bonds or ring structures. When such bonds or ring structures are present, the compounds of the invention can exist as cis and/or trans configurations and as mixtures thereof. For example, when a double bond is present, when the two higher-priority groups (at each side of the double bond) are oriented in the same direction, the stereoisomer is referred to as cis, whereas when the two higher-priority groups are oriented in opposing directions, the stereoisomer is referred to as trans. The term “cis” can also refer to the orientation of two substituents with reference to each other and the plane of the ring (either both “up” or both “down”). Analogously, the term “trans” can also refer to the orientation of two substituents with reference to each other and the plane of the ring (the substituents being on opposite sides of the ring).

Included within the scope of the claimed compounds of the present invention are all stereoisomers, geometric isomers and tautomeric forms of the compounds of the invention, including compounds exhibiting more than one type of isomerism, and mixtures of one or more thereof. Also included are acid addition or base salts wherein the counterion is optically active, for example, D-lactate or L-lysine, or racemic, for example, DL-tartrate or DL-arginine.

### Tautomerism

Where structural isomers are interconvertible via a low energy barrier, tautomeric isomerism (‘tautomerism’) may occur. This may take the form of proton tautomerism in compounds of the invention containing, for example, an imino/amino, keto/enol, or oxime/nitroso group, lactam/lactim or so-called valence tautomerism in compounds which contain an aromatic moiety. It follows that a single compound may exhibit more than one type of isomerism.

It must be emphasized that while, for conciseness, the compounds of the invention have been drawn herein in a single tautomeric form, all possible tautomeric forms are included within the scope of the invention.

It is possible that the intermediates and compounds of the present invention may exist in different tautomeric forms, and all such forms are embraced within the scope of the invention. The term “tautomer” or “tautomeric form” refers to structural isomers of different energies which are interconvertible via a low energy barrier. For example, proton tautomers (also known as prototropic tautomers) include interconversions via migration of a proton, such as keto-enol and imine-enamine isomerizations.

Valence tautomers include interconversions by reorganization of some of the bonding electrons.

### Isotopes

The present invention includes all pharmaceutically acceptable isotopically labelled compounds of the invention wherein one or more atoms are replaced by atoms having the same atomic number, but an atomic mass or mass number different from the atomic mass or mass number usually found in nature.

Examples of isotopes suitable for inclusion in the compounds of the invention include isotopes of hydrogen, such as ²H and ³H, carbon, such as ¹¹C, ¹³C and ¹⁴C, chlorine, such as ³⁶Cl, fluorine, such as ¹⁸F, iodine, such as ¹²³I, ¹²⁴I, and ¹²⁵I, nitrogen, such as ¹³N and ¹⁵N, oxygen, such as ¹⁵O, ¹⁷O and ¹⁸O, phosphorus, such as ³²P, and sulphur, such as ³⁵S.

Certain isotopically labelled compounds of Formula I, for example, those incorporating a radioactive isotope, are useful in drug and/or substrate tissue distribution studies. The radioactive isotopes tritium, i.e., ³H, and carbon-14, i.e., ¹⁴C, are particularly useful for this purpose in view of their ease of incorporation and ready means of detection.

Substitution with heavier isotopes such as deuterium, i.e., ²H, may afford certain therapeutic advantages resulting from greater metabolic stability, for example, increased in vivo half-life or reduced dosage requirements, and hence may be preferred in some circumstances.

In some embodiments, the disclosure provides deuterium-labeled (or deuterated) compounds and salts, where the formula and variables of such compounds and salts are each and independently as described herein. “Deuterated” means that at least one of the atoms in the compound is deuterium in an abundance that is greater than the natural abundance of deuterium (typically approximately 0.015%). A skilled artisan recognized that in chemical compounds with a hydrogen atom, the hydrogen atom actually represents a mixture of H and D, with about 0.015% being D. The concentration of the deuterium incorporated into the deuterium-labeled compounds and salt of the invention may be defined by the deuterium enrichment factor. It is understood that one or more deuterium may exchange with hydrogen under physiological conditions.

In some embodiments, one or more hydrogen atoms on certain metabolic sites on the compounds of the invention may be deuterated. MetaSite (moldiscovery.com/software/metasite/) may be helpful in predicting some metabolic sites on the compounds of the invention.

Substitution with positron-emitting isotopes, such as 11C ¹⁸F, ¹⁵O and ¹³N, can be useful in Positron Emission Tomography (PET) studies for examining substrate receptor occupancy.

Isotopically labelled compounds of the invention can generally be prepared by conventional techniques known to those skilled in the art or by processes analogous to those described in the accompanying Examples and Preparations using an appropriate isotopically labelled reagent in place of the non-labelled reagent previously employed.

Pharmaceutically acceptable solvates (including hydrates) in accordance with the invention include those wherein the solvent of crystallization may be isotopically substituted, e.g., D₂O, d6-acetone, d6-DMSO.

### Salts

The compounds of the present invention may be isolated and used per se, or when possible, in the form of its pharmaceutically acceptable salt. The term “salts” refers to inorganic and organic salts of a compound of the present invention. These salts can be prepared in situ during the final isolation and purification of a compound, or by separately treating the compound with a suitable organic or inorganic acid or base and isolating the salt thus formed.

Salts encompassed within the term “pharmaceutically acceptable salts” refer to the compounds of the invention which are generally prepared by reacting the free base with a suitable organic or inorganic acid to provide a salt of the compound of the invention that is suitable for administration to a patient, or by reacting the free acid with a suitable organic or inorganic base to provide a salt of the compound of the invention that is suitable for administration to a patient.

In addition, the compounds of the invention may also include other salts of such compounds which are not necessarily pharmaceutically acceptable salts, which may be useful as intermediates for one or more of the following: 1) preparing compounds of Formula I; 2) purifying compounds of Formula I; 3) separating enantiomers of compounds of Formula I; or 4) separating diastereomers of compounds of Formula I.

Suitable base salts are formed from bases which form non-toxic salts. Examples include, but are not limited to aluminum, ammonium, arginine, benzathine, calcium, choline, diethylamine, diolamine, glycine, lysine, magnesium, meglumine, olamine, potassium, sodium, tromethamine and zinc salts.

Hemisalts of acids and bases may also be formed, for example, hemisulfate and hemicalcium salts.

For a review on suitable salts, see Paulekun, G. S. et al., Trends in Active Pharmaceutical Ingredient Salt Selection Based on Analysis of the Orange Book Database, J. Med. Chem. 2007; 50(26), 6665-6672.

Pharmaceutically acceptable salts of compounds of the invention may be prepared by methods well known to one skilled in the art, including but not limited to the following procedures
(i) by reacting a compound of the invention with the desired acid or base;
(ii) by removing an acid- or base-labile protecting group from a suitable precursor of a compound of the invention or by ring-opening a suitable cyclic precursor, for example, a lactone or lactam, using the desired acid or base; or
(iii) by converting one salt of a compound of the invention to another. This may be accomplished by reaction with an appropriate acid or base or by means of a suitable ion exchange procedure.

These procedures are typically carried out in solution. The resulting salt may precipitate out and be collected by filtration or may be recovered by evaporation of the solvent.

### Solvates

The compounds of the invention (e.g. a compound of Formula I or pharmaceutically acceptable salts thereof) may exist in unsolvated and solvated forms. The term ‘solvate’ is used herein to describe a molecular complex comprising the compound of the invention and one or more pharmaceutically acceptable solvent molecules, for example, ethanol. The term ‘hydrate’ is employed when said solvent is water.

In addition, the compounds of the invention may also include other solvates of such compounds that are not necessarily pharmaceutically acceptable solvates, which may be useful as intermediates for one or more of the following: 1) preparing compounds of Formula I or their salts; 2) purifying compounds of Formula I or their salts; 3) separating enantiomers of compounds of Formula I or their salts; or 4) separating diastereomers of compounds of Formula I or their salts.

A currently accepted classification system for organic hydrates is one that defines isolated site, channel, or metal-ion coordinated hydrates—see Polymorphism in Pharmaceutical Solids by K. R. Morris (Ed. H. G. Brittain, Marcel Dekker, 1995). Isolated site hydrates are ones in which the water molecules are isolated from direct contact with each other by intervening organic molecules. In channel hydrates, the water molecules lie in lattice channels where they are next to other water molecules. In metal-ion coordinated hydrates, the water molecules are bonded to the metal ion.

When the solvent or water is tightly bound, the complex may have a well-defined stoichiometry independent of humidity. When, however, the solvent or water is weakly bound, as in channel solvates and hygroscopic compounds, the water/solvent content may be dependent on humidity and drying conditions. In such cases, non-stoichiometry will be the norm.

### Complexes

Also included within the scope of the invention are multi-component complexes (other than salts and solvates) wherein the drug and at least one other component are present in stoichiometric or non-stoichiometric amounts. Complexes of this type include clathrates (drug-host inclusion complexes) and co-crystals. The latter are typically defined as crystalline complexes of neutral molecular constituents which are bound together through non-covalent interactions, but could also be a complex of a neutral molecule with a salt. Co-crystals may be prepared by melt crystallization, by recrystallization from solvents, or by physically grinding the components together—see O. Almarsson and M. J. Zaworotko, Chem. Commun., 17, 1889-1896 (2004). For a general review of multi-component complexes, see Haleblian, J. Pharm. Sci., 64 (8), 1269-1288 (1975).

### Prodrugs

Also included within the scope of the invention are prodrugs of the compounds of the invention. A compound of the invention may be administered in the form of a prodrug. Thus, certain derivatives of a compound of the invention which may have little or no pharmacological activity themselves may, when administered into or onto the body, be converted into a compound of the invention having the desired activity, for example by hydrolytic cleavage, particularly hydrolytic cleavage promoted by an esterase or peptidase enzyme. Such derivatives are referred to as ‘prodrugs’. Further information on the use of prodrugs may be found in ‘The Expanding Role of Prodrugs in Contemporary Drug Design and Development, Nature Reviews Drug Discovery, 17, 559-587 (2018) (J. Rautio et al.).

Prodrugs in accordance with the invention may, for example, be produced by replacing appropriate functionalities present in compounds of the invention with certain moieties known to those skilled in the art as ‘pro-moieties’ as described, for example, in ‘Design of Prodrugs’ by H. Bundgaard (Elsevier, 1985).

Thus, a prodrug in accordance with the invention may be (a) an ester or amide derivative of a carboxylic acid when present in a compound of the invention; (b) an ester, carbonate, carbamate, phosphate or ether derivative of a hydroxyl group when present in a compound of the invention; (c) an amide, imine, carbamate or amine derivative of an amino group when present in a compound of the invention; (d) a thioester, thiocarbonate, thiocarbamate or sulfide derivatives of a thiol group when present in a compound of the invention; or (e) an oxime or imine derivative of a carbonyl group when present in a compound of the invention.

Some specific examples of prodrugs in accordance with the invention include:
(i) when a compound of the invention contains a carboxylic acid functionality (—COOH), an ester thereof, such as a compound wherein the hydrogen of the carboxylic acid functionality of the compound is replaced by C₁-C₈ alkyl (e.g., ethyl) or (C₁-C₈ alkyl)C(═O)OCH₂— (e.g., ^{t}BuC(═O)OCH₂—);
(ii) when a compound of the invention contains an alcohol functionality (—OH), an ester thereof, such as a compound wherein the hydrogen of the alcohol functionality of the compound is replaced by —CO(C₁-C₈ alkyl) (e.g., methylcarbonyl) or the alcohol is esterified with an amino acid;
(iii) when a compound of the invention contains an alcohol functionality (—OH), an ether thereof, such as a compound wherein the hydrogen of the alcohol functionality of the compound is replaced by (C₁-C₈ alkyl)C(═O)OCH₂— or —CH₂OP(═O)(OH)₂;
(iv) when a compound of the invention contains an alcohol functionality (—OH), a phosphate thereof, such as a compound wherein the hydrogen of the alcohol functionality of the compound is replaced by —P(═O)(OH)₂ or —P(═O)(O⁻Na⁺)₂ or —P(═O)(O⁻)₂Ca²⁺;
(v) when a compound of the invention contains a primary or secondary amino functionality (—NH₂ or —NHR where R≠H), an amide thereof, for example, a compound wherein, as the case may be, one or both hydrogens of the amino functionality of the compound is/are replaced by (C₁-C₁₀)alkanoyl, —COCH₂NH₂ or the amino group is derivatized with an amino acid;
(vi) when a compound of the invention contains a primary or secondary amino functionality (—NH₂ or —NHR where R≠H), an amine thereof, for example, a compound wherein, as the case may be, one or both hydrogens of the amino functionality of the compound is/are replaced by —CH₂OP(═O)(OH)₂.

Certain compounds of the invention may themselves act as prodrugs of other compounds the invention. It is also possible for two compounds of the invention to be joined together in the form of a prodrug. In certain circumstances, a prodrug of a compound of the invention may be created by internally linking two functional groups in a compound of the invention, for instance by forming a lactone.

### Metabolites

Also included within the scope of the invention are active metabolites of compounds of Formula I (including prodrugs) or their pharmaceutically acceptable salts, that is, compounds formed in vivo upon administration of the drug, often by oxidation or dealkylation. Some examples of metabolites in accordance with the invention include:
(i) where the compound of Formula I or its pharmaceutically acceptable salt contains a methyl group, a hydroxymethyl derivative thereof (—CH₃->-CH₂OH) and
(ii) where the compound of Formula I or its pharmaceutically acceptable salt contains an alkoxy group, a hydroxy derivative thereof (—OR->-OH).

Also included within the scope of the invention are active metabolites of compounds of the invention, that is, compounds formed in vivo upon administration of the drug, often by oxidation or dealkylation. Some examples of metabolites in accordance with the invention include, but are not limited to,
(i) where the compound of the invention contains an alkyl group, a hydroxyalkyl derivative thereof (—CH->-COH):
(ii) where the compound of the invention contains an alkoxy group, a hydroxy derivative thereof (—OR->-OH);
(iii) where the compound of the invention contains a tertiary amino group, a secondary amino derivative thereof (—NRR′->-NHR or —NHR′);
(iv) where the compound of the invention contains a secondary amino group, a primary derivative thereof (—NHR->-NH₂);
(v) where the compound of the invention contains a phenyl moiety, a phenol derivative thereof (-Ph->-PhOH);
(vi) where the compound of the invention contains an amide group, a carboxylic acid derivative thereof (—CONH₂->COOH); and
(vii) where the compound contains a hydroxy or carboxylic acid group, the compound may be metabolized by conjugation, for example with glucuronic acid to form a glucuronide. Other routes of conjugative metabolism exist. These pathways are frequently known as Phase 2 metabolism and include, for example, sulfation or acetylation. Other functional groups, such as NH groups, may also be subject to conjugation.

### Solid Form

The compounds of the invention may exist in a continuum of solid states ranging from fully amorphous to fully crystalline. The term ‘amorphous’ refers to a state in which the material lacks long range order at the molecular level and, depending upon temperature, may exhibit the physical properties of a solid or a liquid. Typically, such materials do not give distinctive X-ray diffraction patterns and, while exhibiting the properties of a solid, are more formally described as a liquid. Upon heating, a change from solid to liquid properties occurs which is characterized by a change of state, typically second order (‘glass transition’). The term ‘crystalline’ refers to a solid phase in which the material has a regular ordered internal structure at the molecular level and gives a distinctive X-ray diffraction pattern with defined peaks. Such materials when heated sufficiently will also exhibit the properties of a liquid, but the change from solid to liquid is characterized by a phase change, typically first order (‘melting point’).

The compounds of the invention may also exist in a mesomorphic state (mesophase or liquid crystal) when subjected to suitable conditions. The mesomorphic state is intermediate between the true crystalline state and the true liquid state (either melt or solution) and consists of two dimensional order on the molecular level. Mesomorphism arising as the result of a change in temperature is described as ‘thermotropic’ and that resulting from the addition of a second component, such as water or another solvent, is described as ‘lyotropic’. Compounds that have the potential to form lyotropic mesophases are described as ‘amphiphilic’ and consist of molecules which possess an ionic (such as —COO⁻Na⁺, —COO⁻K⁺, or —SO₃⁻Na⁺) or non-ionic (such as —N⁻N⁺(CH₃)₃) polar head group. For more information, see Crystals and the Polarizing Microscope by N. H. Hartshorne and A. Stuart, 4^{th} Edition (Edward Arnold, 1970).

Certain compounds of the present invention may exist in more than one crystal form (generally referred to as “polymorphs”). Polymorphs may be prepared by crystallization under various conditions, for example, using different solvents or different solvent mixtures for recrystallization; crystallization at different temperatures; and/or various modes of cooling, ranging from very fast to very slow cooling during crystallization. Polymorphs may also be obtained by heating or melting the compound of the present invention followed by gradual or fast cooling. The presence of polymorphs may be determined by solid probe NMR spectroscopy, IR spectroscopy, differential scanning calorimetry, powder X-ray diffraction or such other techniques.

In general the compounds of this invention can be made by processes which include processes analogous to those known in the chemical arts, particularly in light of the description contained herein. Certain processes for the manufacture of the compounds of this invention are provided as further features of the invention and are illustrated by the following reaction schemes. Other processes may be described in the experimental section. Specific synthetic schemes for preparation of the compounds of Formula I or their pharmaceutically acceptable salts are outlined below. Note that tetrazoles are generally a high-energy functional group and care should be taken in the synthesis and handling of tetrazole-containing molecules.

### Synthesis

Compounds of the present invention may be synthesized by synthetic routes that include processes analogous to those well-known in the chemical arts, particularly in light of the description contained herein. The starting materials are generally available from commercial sources or may be prepared using methods well known to those skilled in the art. Many of the compounds used herein are related to, or may be derived from, compounds in which one or more of the scientific interest or commercial need has occurred. Accordingly, such compounds may be one or more of 1) commercially available; 2) reported in the literature or 3) prepared from other commonly available substances by one skilled in the art using materials which have been reported in the literature.

For illustrative purposes, the reaction schemes depicted below provide potential routes for synthesizing the compounds of the present invention as well as key intermediates. For a more detailed description of the individual reaction steps, see the Examples section below. Those skilled in the art will appreciate that other synthetic routes may be used to synthesize the inventive compounds. Although specific starting materials and reagents are discussed below, other starting materials and reagents may be substituted to provide one or more of a variety of derivatives or reaction conditions. In addition, many of the compounds prepared by the methods described below may be further modified in light of this disclosure using conventional chemistry well known to those skilled in the art.

The skilled person will appreciate that the experimental conditions set forth in the schemes that follow are illustrative of suitable conditions for effecting the transformations shown, and that it may be necessary or desirable to vary the precise conditions employed for the preparation of compounds of the invention. It will be further appreciated that it may be necessary or desirable to carry out the transformations in a different order from that described in the schemes, or to modify one or more of the transformations, to provide the desired compound of the invention. In the preparation of compounds of the invention it is noted that some of the preparation methods useful for the preparation of the compounds described herein may require protection of remote functionality (e.g., a primary amine, secondary amine, carboxyl, etc. in a precursor of a compound of the invention). The need for such protection will vary depending on the nature of the remote functionality and the conditions of the preparation methods. The need for such protection is readily determined by one skilled in the art. The use of such protection/deprotection methods is also within the skill in the art. For a general description of protecting groups and their use, see March's Advanced Organic Chemistry: Reactions, Mechanisms, and Structure 8th Edition.

For example, if a compound contains a amine or carboxylic acid functionality, such functionality may interfere with reactions at other sites of the molecule if left unprotected. Accordingly, such functionalities may be protected by an appropriate protecting group (PG) which may be removed in a subsequent step. Suitable protecting groups for amine and carboxylic acid protection include those protecting groups commonly used in peptide synthesis (such as N-t-butoxycarbonyl (Boc), benzyloxycarbonyl (Cbz), and 9-fluorenylmethylenoxycarbonyl (Fmoc) for amines and lower alkyl or benzyl esters for carboxylic acids) which are generally not chemically reactive under the reaction conditions described and may typically be removed without chemically altering other functionality in a compound of the invention.

Reactions can be monitored according to any suitable method known in the art. For example, product formation can be monitored by spectroscopic means, such as nuclear magnetic resonance spectroscopy (e.g., ¹H or ¹³C), infrared spectroscopy, spectrophotometry (e.g., UV-visible), mass spectrometry, or by chromatographic methods such as high-performance liquid chromatography (HPLC) or thin-layer chromatography (TLC).

Compounds of Formula I, salts and intermediates thereof may be prepared according to the following reaction schemes and accompanying discussion. The reaction schemes described below are intended to provide a general description of the methodology employed in the preparation of the compounds of the present invention. Some of the compounds of the present invention contain a single chiral center with stereochemical designation (R or S) and others will contain two separate chiral centers with stereochemical designation (R or S). It will be apparent to one skilled in the art that most of the synthetic transformations can be conducted in a similar manner whether the materials are enantioenriched or racemic. Moreover, the resolution to the desired optically active material may take place at any desired point in the sequence using well known methods such as described herein and in the chemistry literature.

Unless otherwise indicated, in the reaction schemes that follow, variables R, R¹, R², R³, R^{3a}, R^{A}, L¹, T¹, T², T³, T⁴, T⁵, T⁶, T⁷, T⁸, T⁹, T¹⁰, T¹¹, T¹², t1, and t2 and structural Formula I (including, e.g., Formula Ia) in the reaction schemes and discussion that follow are as defined herein or consistent with those described in the claims and embodiments herein. For each of the variables, its meaning remains the same as initially described unless otherwise indicated in a later occurrence. In general, the compounds of this invention may be made by processes which include processes analogous to those known in the chemical arts, particularly in light of the description contained herein. Certain processes for the manufacture of the compounds of this invention and intermediates thereof are provided as further features of the invention and are illustrated by the following reaction schemes. Other processes are described in the experimental section. The schemes and examples provided herein (including the corresponding description) are for illustration only, and not intended to limit the scope of the present invention.

In general, the compounds of this invention may be made by processes described herein and by analogous processes known to those skilled in the art. Certain processes for the manufacture of the compounds of this invention are described in the following reaction schemes. Other processes are described in the experimental section. The schemes and examples provided herein (including the corresponding description) are for illustration only. One skilled in the art will recognize that intermediates and compounds of Formula I prepared according to the following schemes may be isolated as salts or non-salts depending on the conditions of the reaction, isolation, or purification. One skilled in the art will also recognize that in some instances, additional synthetic steps may be required to protect and deprotect certain functional groups present within the synthetic sequence. One skilled in the art will further recognize that in other instances, certain functional groups may be carried through the synthetic sequences described and then may be transformed into alternate substituents present in compounds of Formula I.

Scheme 1 refers to the preparation of compounds of Formula I from amino acids of structure 1-1. Compounds of structure 1-1 can be reacted with isocyanates of structure 1-2 in the presence of a base (such as N,N-diisopropylethylamine) in a suitable solvent (such as tetrahydrofuran) to afford ureas of structure 1-4. Alternatively, ureas of structure 1-4 can be obtained by reacting amines of structure 1-3 with a suitable reactant (such as triphosgene or 1,1′-carbonyldiimidazole) to form an intermediate and subsequently reacting the resulting intermediate with a compound of structure 1-1. One skilled in the art will recognize that numerous alternate conditions may be selected for the formation of ureas (such as ureas of structure 1-4). (See e.g. J. Med. Chem. 2020, 63, 2751-2788). Compounds of Formula I can be prepared from an amide bond forming reaction between carboxylic acid intermediate 1-4 and amine intermediate 1-5. Amide bond forming reactions of this type can be achieved by combining a carboxylic acid (such as carboxylic acid structure 1-4) with an amine (such as amine of structure 1-5) in the presence of an activating reagent (such as 1-ethyl-3-(3-dimethylaminopropyl)-carbodiimide and 1-hydroxybenzotriazole) and a base (such as N,N-diisopropylethylamine) in a suitable solvent (such as dichloromethane). One skilled in the art will recognize that numerous alternate conditions may be selected for the formation of an amide (such as a compound of Formula I) from a carboxylic acid (such as a carboxylic acid of structure 1-4) and an amine (such as an amine of structure 1-5). (See e.g. Chem. Rev. 2011, 111, 6557-6602).

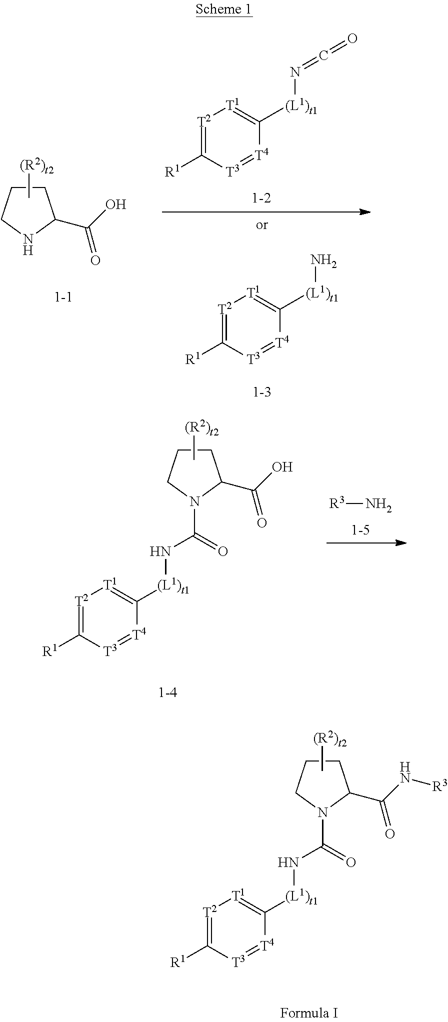

Scheme 2 refers to preparation of compounds of Formula I from amino esters of structure 2-1 wherein R is alkyl, cycloalkyl, cycloalkylalkyl, benzyl, or the like. Amino esters of structure 2-1 can be reacted with isocyanates of structure 1-2 in the presence of a base (such as N,N-diisopropylethylamine) in a suitable solvent (such as tetrahydrofuran) to afford ureas of structure 2-2. Alternatively, ureas of structure 2-2 can also be obtained by reacting amines of structure 1-3 with a suitable reactant (such as triphosgene or 1,1′-carbonyldiimidazole) to form an intermediate and subsequently reacting the resulting intermediate with an amino ester of structure 2-1. The ester in the ureas of structure 2-2 can be converted to a carboxylic acid via methods known in the art. The conditions selected for conversion of an ester to an acid are dependent on the type of ester present. For example, a compound of structure 2-2 wherein R is methyl (i.e. having a methyl ester functional group) can be converted to a carboxylic acid upon treatment with lithium hydroxide in a solvent mixture consisting of tetrahydrofuran and water. Alternatively, a compound of structure 2-2 wherein R is t-butyl (i.e. having a tert-butyl ester functional group) can be converted to a carboxylic acid upon treatment with an acid such as trifluoroacetic acid. The resulting acid in each case can be further transformed via an amide bond forming reaction to afford compounds of Formula I (as described in Scheme 1).

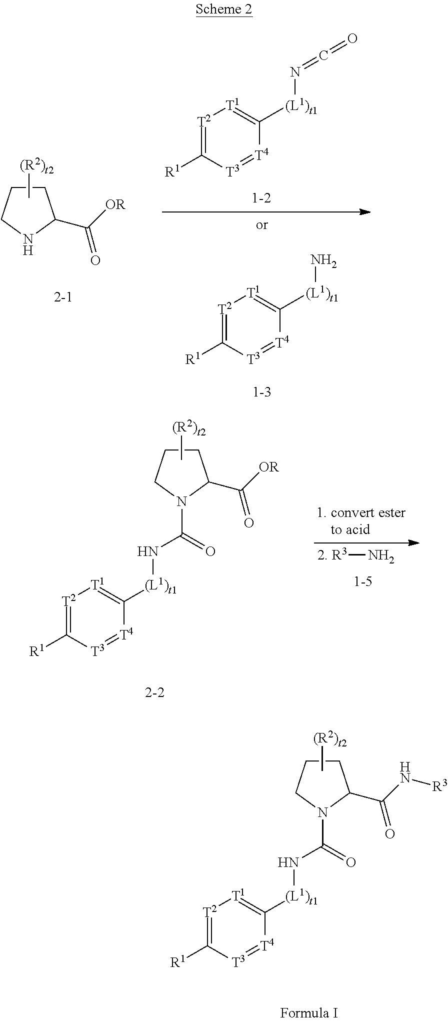

Scheme 3 refers to the preparation of compounds of Formula I from nitrogen-protected amino acids of structure 3-1. A carboxylic acid of structure 3-1 can be reacted with an amine of structure 1-5 via amide bond forming conditions as described in Scheme 1. The remaining tert-butyloxycarbonyl protecting group in the resulting amide can be removed upon treatment with an acid such as trifluoroacetic acid to afford the amine intermediate of structure 3-2. The intermediate of structure 3-2 can be coupled with an isocyanate of structure 1-2 or an amine of structure 1-3 via urea-forming conditions as described previously in Schemes 1 and 2 to afford compounds of Formula I. One skilled in the art would recognize that an alternative protecting group from the Boc shown in structure 3-1 may also be used. For example, fluorenylmethyloxycarbonyl (Fmoc), another protecting group, could be used in place of the Boc group of structure 3-1. After amidation, the Fmoc can be subsequently removed by conditions known to one skilled in the art, such as stirring with piperidine in a solvent such as N,N-dimethylformamide.

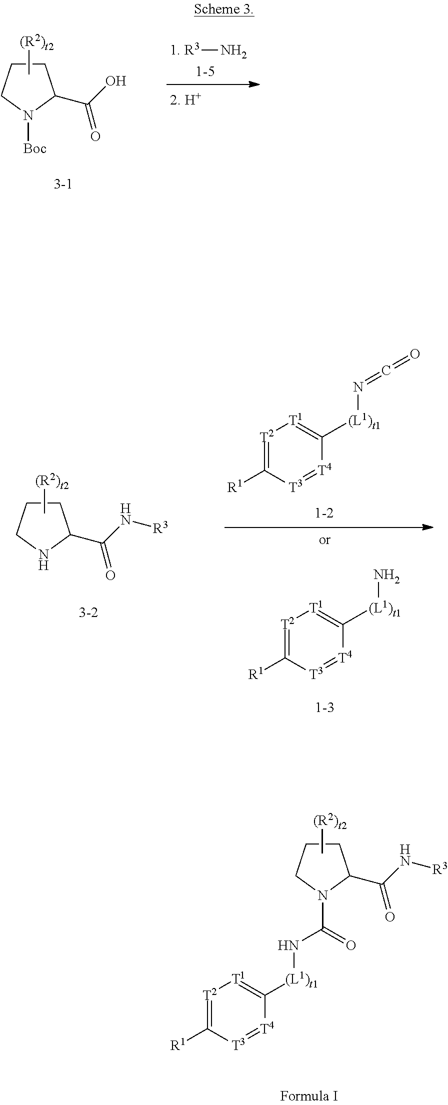

In some instances, preparation of the amine R³NH₂ (structure 1-5) is needed for the preparation of compounds of Formula I. Scheme 4 outlines an example preparation of an amine of structure 4-5 (a representative example of an amine of structure of 1-5) and subsequent transformation to afford compounds of Formula I. Compounds of structures 4-1 and 4-2 can be coupled with compounds of structures 4-3 and 4-4, respectively, via a Suzuki reaction (Acc Chem Res. 2008, 1461-1473) to afford intermediates 4-5. A Suzuki reaction is the coupling of an aryl or vinyl boronic acid with an aryl or vinyl halide using a palladium catalyst. For example, an aryl bromide of structure 4-1 can be reacted with an aryl boronic acid of structure 4-3 in the presence of a base (such as sodium carbonate) and a palladium catalyst (such as [(di(1-adamantyl)-butylphosphine)-2-(2′-amino-1,1′-biphenyl)]palladium(II) methanesulfonate) in a suitable solvent (such as 1,4-dioxane) to afford the intermediate of structure 4-5. The intermediate of structure 4-5 is an example of a compound of structure 1-5 (R³NH₂). The intermediate of structure 4-5 can be further transformed according to the methods described in Schemes 1-3 to afford compounds of Formula I wherein R³ is R^{3a}.

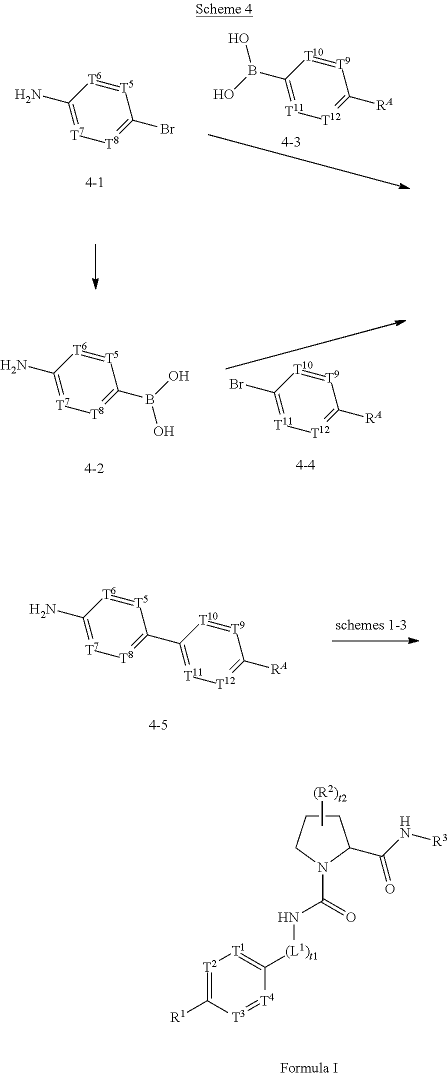

Scheme 5 refers to the preparation of compounds of Formula I whereby the R³ group (and wherein R³ is R^{3a}) is constructed in a stepwise fashion. Intermediates of structure 1-4 (which can be prepared according to Scheme 1 or Scheme 2) can be reacted with amines of structure 4-1 via an amide bond forming reaction according to methods described in Scheme 1 to afford the intermediate of structure 5-1. An aryl halide or heteroaryl halide of structure 5-1 can be coupled with an aryl boronic acid or heteroaryl boronic acid of structure 4-3 via a Suzuki reaction to afford a compound of Formula 1. For example, an intermediate of structure 5-1 can be reacted with an aryl boronic acid or heteroaryl boronic acid of structure 4-3 in the presence of a base (such as sodium carbonate) and a palladium catalyst (such as [(di(1-adamantyl)-butylphosphine)-2-(2′-amino-1,1′-biphenyl)]palladium(II) methanesulfonate) in a suitable solvent (such as 1,4-dioxane) to afford a compound of Formula I. Alternatively, an intermediate of structure 1-4 can be reacted with an amine of structure 5-2 via an amide bond forming reaction according to methods described in Scheme 1 to afford an intermediate of structure 5-3. Additionally, an intermediate 5-3 can also be prepared by reacting intermediate of structure 5-1 with 4,4,4′,4′,5,5,5′,5′-octamethyl-2,2′-bi-1,3,2-dioxaborolane in the presence of a base (such as potassium acetate), a ligand (such as 2-dicyclohexylphosphino-2′,4′,6′-triisopropylbiphenyl), and a palladium catalyst (such as chloro(2-dicyclohexylphosphino-2′,4′,6′-triisopropyl-1,1′-biphenyl)[2-(2′-amino-1,1′-biphenyl)]palladium(II)) in a suitable solvent (such as 2-methyltetrahydrofuran). The intermediate of structure 5-3 can be coupled with an aryl halide or heteroaryl halide of structure 4-4 via a Suzuki reaction to afford a compound of Formula I. For example, the intermediate of structure 5-3 can be reacted with an aryl halide or heteroaryl halide of structure 4-4 in the presence of a base (such as sodium carbonate) and a palladium catalyst (such as [(di(1-adamantyl)-butylphosphine)-2-(2′-amino-1,1′-biphenyl)]palladium(II) methanesulfonate) in a suitable solvent (such as 1,4-dioxane) to afford a compound of Formula I wherein R³ is R^{3a}.

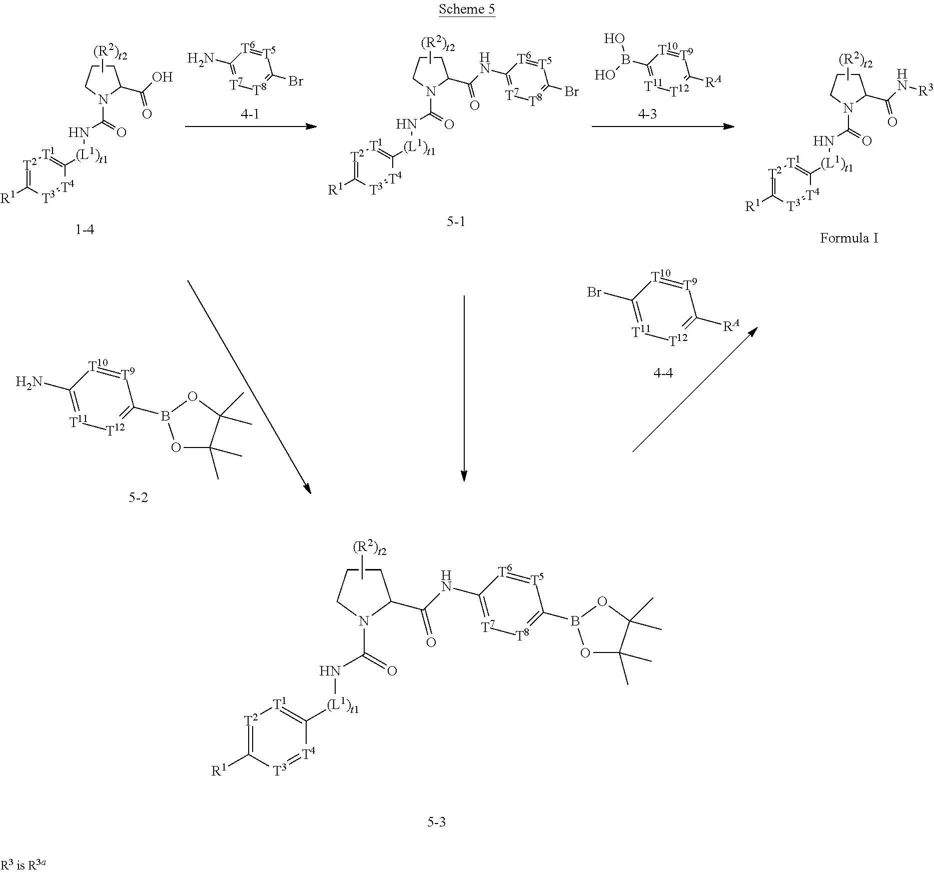

Scheme 6 refers to the preparation of non-racemic compounds of Formula Ia. Non-racemic compounds (such as compounds of structure 1-1a, 2-1a, and 3-1a) can be transformed according to the methods described in Schemes 1-5 and 7-10 to afford non-racemic compounds of Formula Ia. One skilled in the art will recognize that the enantiomeric purity observed for the compound of Formula Ia can be influenced by numerous factors (such as the synthetic sequence utilized, the reagents selected for each transformation, and the purification methods employed).

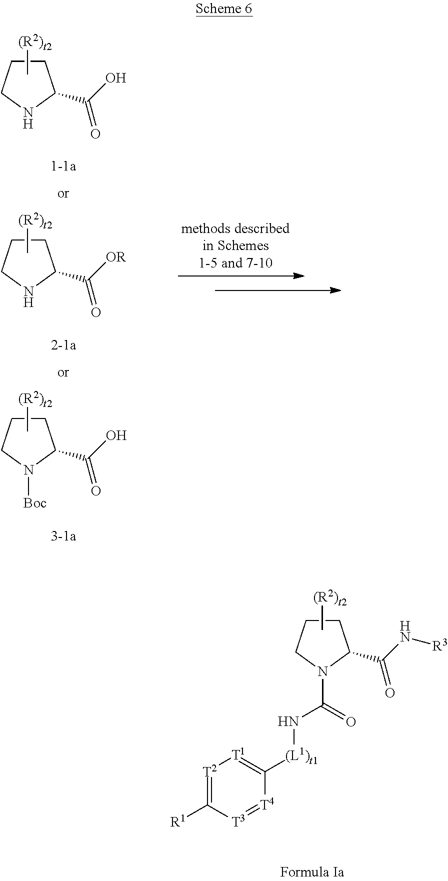

Scheme 7 refers to the preparation of compounds of Formula I wherein R³ is R³N from nitrogen-protected amino amides of structure 7-1. The tert-butyloxycarbonyl protecting group can first be removed upon treatment with an acid such as trifluoroacetic acid to afford the amine intermediate, which can subsequently be coupled with isocyanate of structure 1-2 or amine of structure 1-3 via urea-forming conditions as described previously in Schemes 1 and 2, to afford intermediates 1-4. An aryl halide or heteroaryl halide of structure 1-4 can be coupled with an aryl boronic acid or heteroaryl boronic acid of structure 4-3 via a Suzuki reaction to afford a compound of Formula I. For example, the reaction of 1-4 and 4-3 can occur in the presence of a base (such as sodium carbonate) and a palladium catalyst (such as [(di(1-adamantyl)-butylphosphine)-2-(2′-amino-1,1′-biphenyl)]palladium(II) methanesulfonate) in a suitable solvent (such as 1,4-dioxane) to afford a compound of Formula I wherein R³ is R^{3a}.

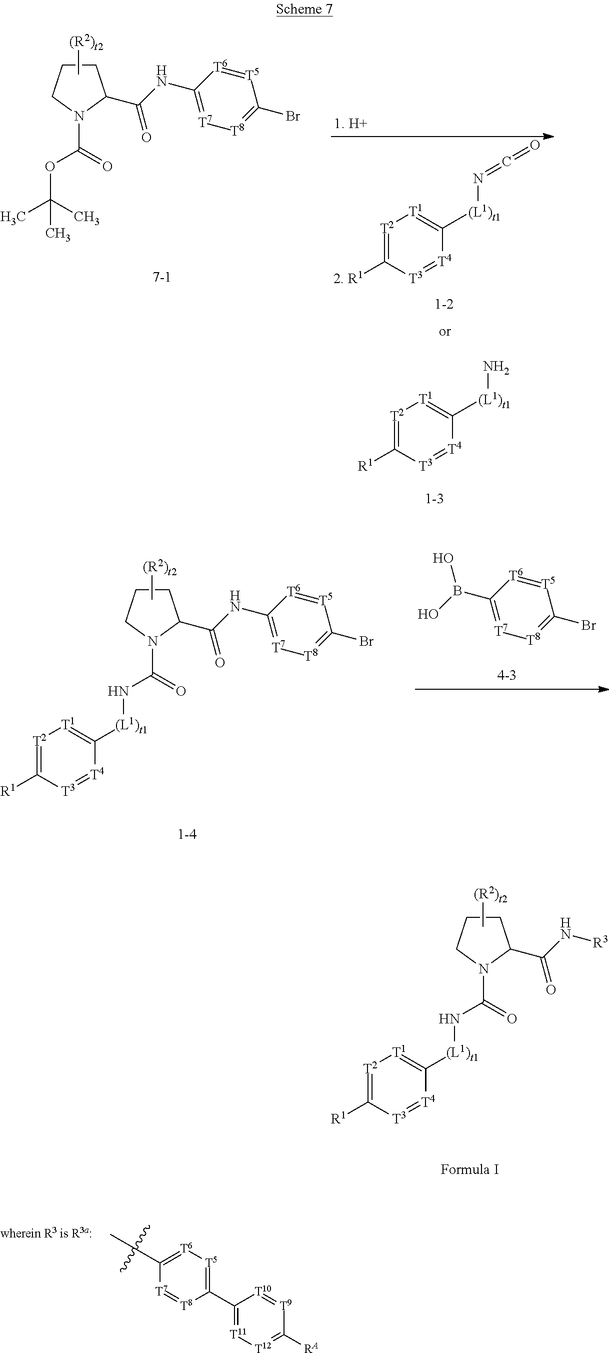

In some instances, a protecting group on a carboxylic acid, such as an ester, is needed for the preparation of compounds of Formula I wherein R^{A} is —C(═O)OH [for example, wherein R³ is R^{3a} and R^{A} is —C(═O)OH]. Scheme 8 outlines an example of preparation of an ester 8-2 from acid 8-1. Ester 8-2 can be prepared by reaction with tert-butanol in the presence of pyridine and 4-methylbenzene-1-sulfonyl chloride as well as other methods. The aryl halide or heteroaryl halide of structure 8-2 can be coupled with an aryl boronic acid or heteroaryl boronic acid of structure 4-2 via a Suzuki reaction to afford intermediate 8-3. For example, the reaction of 8-2 and 4-2 can occur in the presence of a base (such as sodium carbonate) and a palladium catalyst (such as [(di(1-adamantyl)-butylphosphine)-2-(2′-amino-1,1′-biphenyl)]palladium(II) methanesulfonate) in a suitable solvent (such as 1,4-dioxane). Intermediate 8-3 can then be converted to structure 8-4 by an amidation reaction as shown in Schemes 1-4. A final ester hydrolysis of 8-4 using an acid such as trifluoroacetic acid would afford compounds of Formula I wherein R³ is R^{3a} and R^{A} is —C(═O)OH. One skilled in the art will recognize that other esters besides tert-butyl may be used in variants of compound 8-2.

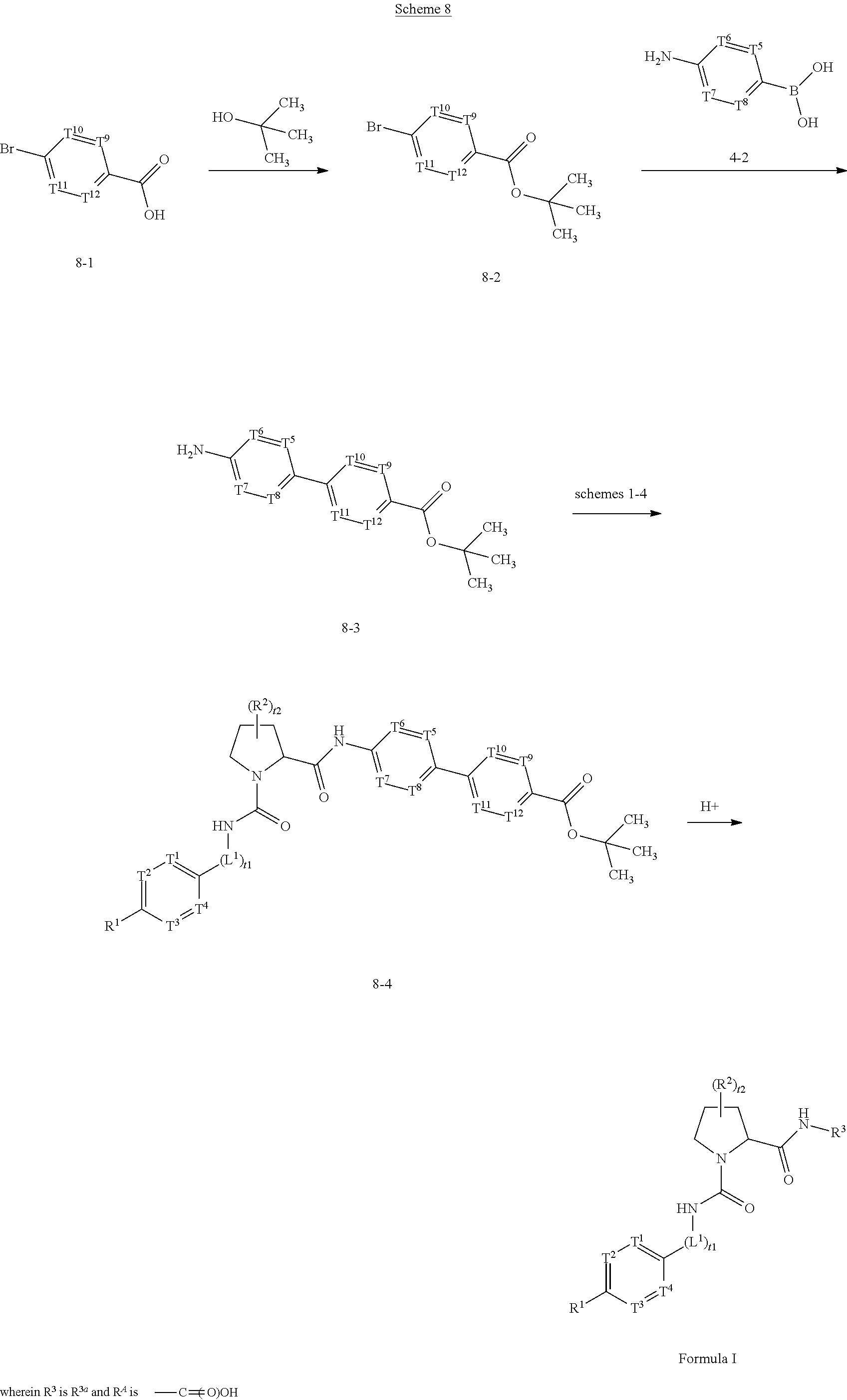

Scheme 9 details another sequence of bond formation to arrive at compounds of Formula I wherein R³ is R^{3a} [including, for example, those with R^{A} being —C(═O)OH]. Boc-protected amino acid 3-1 can be reacted with amines of structure 4-1 via an amide bond forming reaction according to methods described in Scheme 1 to afford intermediate of structure 7-1. The aryl halide or heteroaryl halide of structure 7-1 can be coupled with an aryl boronic acid or heteroaryl boronic acid of structure 4-3 via a Suzuki reaction to afford intermediate 9-2. For example, an intermediate of structure 7-1 can be reacted with an aryl boronic acid or heteroaryl boronic acid of structure 4-3 in the presence of a base (such as sodium carbonate) and a palladium catalyst (such as [(di(1-adamantyl)-butylphosphine)-2-(2′-amino-1,1′-biphenyl)]palladium(II) methanesulfonate) in a suitable solvent (such as 1,4-dioxane) to afford 9-2. Alternatively, Boc-protected amino acid 3-1 can be reacted with amines of structure 4-2 via an amide bond forming reaction according to methods described in Scheme 1 to afford intermediate of structure 9-1. The aryl or heteroaryl boronic acid of structure 9-1 can be coupled with an aryl halide or heteroaryl halide of structure 4-4 via a Suzuki reaction to afford intermediate 9-2. The tert-butyloxycarbonyl protecting group of 9-2 can then be removed upon treatment with an acid such as trifluoroacetic acid to afford the amine intermediate, which can subsequently be coupled with an isocyanate of structure 1-2 or amine of structure 1-3, via urea-forming conditions as described previously in Schemes 1-3 and 7, to afford compounds of Formula I wherein R³ is R^{3a}. Other variations in substitution pattern of structures 4-3 and 4-4 can also be synthesized using Scheme 9, such as meta-substituted R^{A} groups (in that case, a compound of Formula I wherein R³ is R^{3b} can be made).

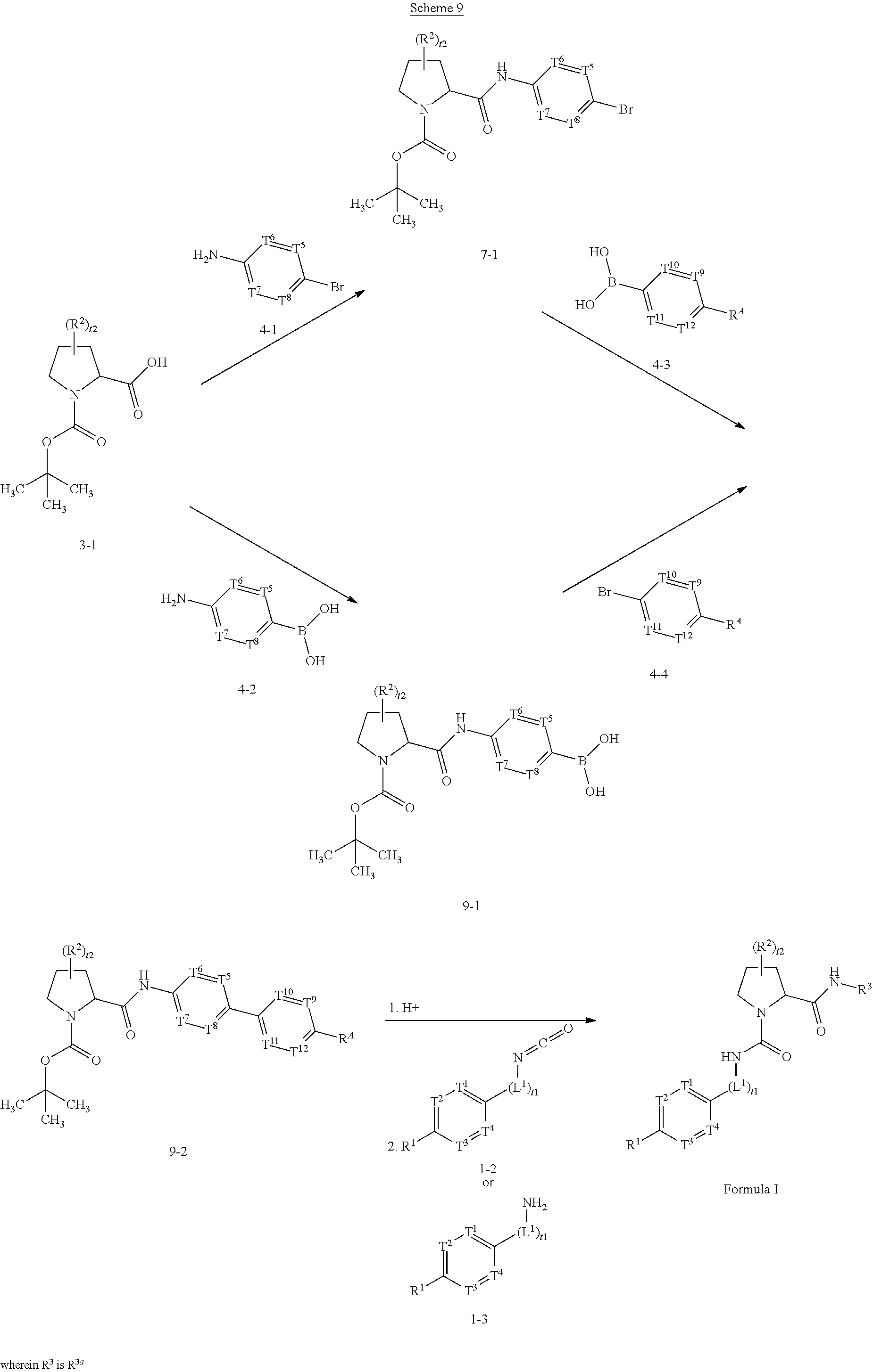

Scheme 10 details another reaction type to arrive at compounds of Formula I. Amino amide 3-2, which may contain an ester protecting group on a carboxylic acid as part of R³, can be reacted with carboxylic acids of structure 10-1 via a Curtius rearrangement to afford the urea bond-containing intermediate. This Curtius rearrangement can be enacted in the presence of diphenyl phosphorazidate and a base such as triethylamine in an organic solvent such as toluene. The resulting intermediate, if R³ contains an ester protecting group, can be deprotected using an acid such as trifluoroacetic acid to afford compounds of Formula I.

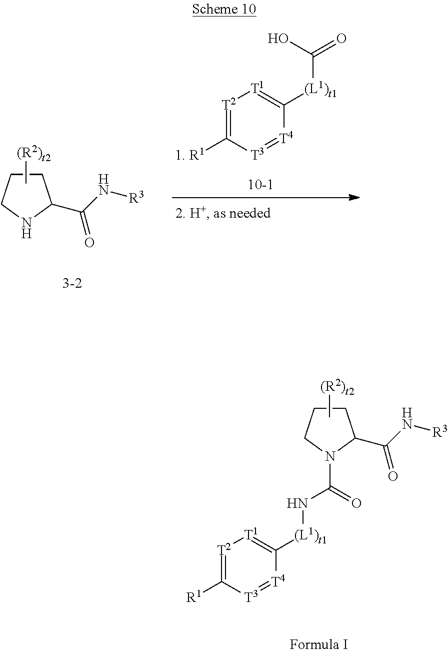

A detailed description of the individual reaction steps is provided in the Example section below. Those skilled in the art will appreciate that other synthetic routes may be used to synthesize the compounds. Although specific starting materials and reagents are discussed below, other starting materials and reagents can be easily substituted to provide a variety of derivatives and/or reaction conditions. In addition, many of the compounds prepared by the methods described below can be further modified in light of this disclosure using conventional chemistry well known to those skilled in the art.

### Co-Administration

The compounds of the invention may be used alone, or in combination with one or more other therapeutic agents. The invention provides any of the uses, methods or compositions as defined herein wherein the compound of the invention, or pharmaceutically acceptable salt thereof, is used in combination with one or more other therapeutic agent discussed herein.

The administration of two or more compounds “in combination” means that all of the compounds are administered closely enough in time to affect treatment of the subject. The two or more compounds may be administered simultaneously or sequentially, via the same or different routes of administration, on same or different administration schedules and with or without specific time limits depending on the treatment regimen. Additionally, simultaneous administration may be carried out by mixing the compounds prior to administration or by administering the compounds at the same point in time but as separate dosage forms at the same or different site of administration. Examples of “in combination” include, but are not limited to, “concurrent administration,” “co-administration,” “simultaneous administration,” “sequential administration” and “administered simultaneously”.

A compound of the invention and the one or more other therapeutic agents may be administered as a fixed or non-fixed combination of the active ingredients. The term “fixed combination” means a compound of the invention, or a pharmaceutically acceptable salt thereof, and the one or more therapeutic agents, are both administered to a subject simultaneously in a single composition or dosage. The term “non-fixed combination” means that a compound of the invention, or a pharmaceutically acceptable salt thereof, and the one or more therapeutic agents are formulated as separate compositions or dosages such that they may be administered to a subject in need thereof simultaneously or at different times with variable intervening time limits, wherein such administration provides effective levels of the two or more compounds in the body of the subject.

The combination agents are administered to a patient (e.g. a mammal or human) in a therapeutically effective amount. By “therapeutically effective amount” it is meant an amount of a compound of the present invention that, when administered alone or in combination with an additional therapeutic agent to a mammal, is effective to treat the desired disease/disorder/condition (e.g., T2DM or obesity).

In some embodiments, a compound of this invention may be co-administered with one or more other agents such as Orlistat, TZDs and other insulin-sensitizing agents, FGF21 analogs, Metformin, Omega-3-acid ethyl esters (e.g., Lovaza), Fibrates, HMG CoA-reductase Inhibitors, Ezetimibe, Probucol, Ursodeoxycholic acid, TGR5 agonists, FXR agonists, Vitamin E, Betaine, Pentoxifylline, CB1 antagonists, Carnitine, N-acetylcysteine, Reduced glutathione, lorcaserin, the combination of naltrexone with buproprion, SGLT2 inhibitors (including dapagliflozin, canagliflozin, empagliflozin, tofogliflozin, ertugliflozin, ASP-1941, THR1474, TS-071, ISIS388626 and LX4211 as well as those in WO2010023594), Phentermine, Topiramate, GLP-1 receptor agonists, GIP receptor agonists, GIP receptor inhibitors and/or antagonists, dual GLP-1 receptor/glucagon receptor agonists (e.g., OPK88003, MED10382, JNJ-64565111, NN9277, BI 456906), dual GLP-1 receptor/GIP receptor agonists [e.g., Tirzepatide (LY3298176), NN9423, NN9541, HS-20094, SCO-094, VK2735, CT-388, GMA-106, CT-868, HRS9531], dual GLP-1 receptor/glucagon receptor agonists (e.g. DD-01, PB-718, mazdutide, pemvidutide, pegapamodutide, survodutide, LM-008, IBI-362, AZD9550), dual GLP-1 receptor/GLP-2 receptor agonists (e.g. dapiglutide), dual GLP-1 receptor/amylin receptor agonists (e.g. amycretin), cagrilinitide/semaglutide, GLP-1 receptor agonist/GIP receptor antagonist (maridebart cafraglutide), dual GLP-1 receptor/FGF21 receptor agonists (e.g. HEC-88473, BI 3006337), triple agonists of the GLP-1 receptor/glucagon receptor/GIP receptor (e.g. retatrutide), triple agonists of the GLP-1 receptor/glucagon receptor/FGF21 receptor (e.g. DR10624), NPY2 receptor agonists (e.g. BI 1820237), activin receptor type-2B modulators (e.g. bimagrumab), amylin receptor agonists, GPR75 modulators, delta-5 desaturase inhibitors, orexin 2 receptor modulators, Angiotensin-receptor blockers, an acetyl-CoA carboxylase (ACC) inhibitor, a ketohexokinase (KHK) inhibitor, ASK1 inhibitors, branched-chain alpha-keto acid dehydrogenase kinase inhibitors (BCKDK inhibitors), inhibitors of CCR2 and/or CCR5, PNPLA3 inhibitors, DGAT1 inhibitors, DGAT2 inhibitors, an FGF21 analog, FGF19 analogs, PPAR agonists, FXR agonists, AMPK activators [e.g., ETC-1002 (bempedoic acid)], SCD1 inhibitors or MPO inhibitors.

Exemplary GLP-1 receptor agonists include liraglutide, albiglutide, exenatide, lixisenatide, dulaglutide, semaglutide, danuglipron, orforglipron, lotiglipron, PF-06954522, HM15211, LY3298176, Medi-0382, NN-9924, TTP-054, TTP-273, efpeglenatide, CT-996, ECC5004, XW004, XW014, MDR-001, ZT002, KN-056, GL0034, GSBR-1290, noiiglutide, RGT-075, TTP-273, HRS-7535, GMA-105, TG103, GZR-18, GX-G6, ecnoglutide, PB-119, QLG2065, beinaglutide, those described in WO2018109607, those described in WO2019239319 (PCT/IB2019/054867 filed Jun. 11, 2019), and those described in WO2019239371 (PCT/IB2019/054961 filed Jun. 13, 2019).

Exemplary ACC inhibitors include 4-(4-[(1-isopropyl-7-oxo-1,4,6,7-tetrahydro-1′H-spiro[indazole-5,4′-piperidin]-1′-yl)carbonyl]-6-methoxypyridin-2-yl)benzoic acid, gemcabene, and firsocostat (GS-0976) and phamaceutally acceptable salts thereof.

Exemplary FXR agonists include tropifexor (2-[(1R,3R,5S)-3-({5-cyclopropyl-3-[2-(trifluoromethoxy)phenyl]-1,2-oxazol-4-yl}methoxy)-8-azabicyclo[3.2.1]octan-8-yl]-4-fluoro-1,3-benzothiazole-6-carboxylic acid), cilofexor (GS-9674), obeticholic acid, LY2562175, Met409, TERN-101 and EDP-305 and pharmaceutically acceptable salts thereof.

Exemplary KHK inhibitors include [(1R,5S,6R)-3-{2-[(2S)-2-methylazetidin-1-yl]-6-(trifluoromethyl)pyrimidin-4-yl}-3-azabicyclo[3.1.0]hex-6-yl]acetic acid and pharmaceutically acceptable salts thereof.

Exemplary DGAT2 inhibitors include (S)-2-(5-((3-ethoxypyridin-2-yl)oxy)pyridin-3-yl)-N-(tetrahydrofuran-3-yl)pyrimidine-5-carboxamide [including its crystalline solid forms (Form 1 and Form 2)]. See U.S. Pat. No. 10,071,992.

Some exemplary BCKDK inhibitors include those described in U.S. Pat. Nos. 11,542,270 and 11,059,833, including the following:
5-(5-chloro-4-fluoro 3-methylthiophen-2-yl)-1H-tetrazole;
5-(5-chloro-3-difluoromethylthiophen-2-yl)-1H-tetrazole;
5-(5-fluoro-3-methylthiophen-2-yl)-1H-tetrazole;
5-(5-chloro-3-methylthiophen-2-yl)-1H-tetrazole;
5-(3,5-dichlorothiophen-2-yl)-1H-tetrazole;
5-(4-bromo-3-methylthiophen-2-yl)-1H-tetrazole;
5-(4-bromo-3-ethylthiophen-2-yl)-1H-tetrazole;
5-(4-chloro-3-ethylthiophen-2-yl)-1H-tetrazole;
3-chloro-5-fluorothieno[3,2-b]thiophene-2-carboxylic acid;
3-bromo-5-fluorothieno[3,2-b]thiophene-2-carboxylic acid;
3-(difluoromethyl)-5-fluorothieno[3,2-b]thiophene-2-carboxylic acid;
5,6-difluorothieno[3,2-b]thiophene-2-carboxylic acid; and
3,5-difluorothieno[3,2-b]thiophene-2-carboxylic acid;
   or a pharmaceutically acceptable salt thereof.

Some additional exemplary BCKDK inhibitors include those described in U.S. patent application Ser. No. 18/060,027, filed Nov. 30, 2022, including the following:
6-fluoro-3-(2,4,6-trifluoro-3-methoxyphenyl)-1-benzothiophene-2-carboxylic acid;
6-fluoro-3-(2,4,5-trifluoro-3-methoxyphenyl)-1-benzothiophene-2-carboxylic acid;
6-chloro-3-(2,4,5-trifluoro-3-methylphenyl)-1-benzothiophene-2-carboxylic acid;
6-chloro-3-(2,4-difluoro-3-methoxyphenyl)-1-benzothiophene-2-carboxylic acid;
3-(6-chloro-2,4-difluoro-3-methoxyphenyl)-6-fluoro-1-benzothiophene-2-carboxylic acid;
3-(6-chloro-2,4-difluoro-3-methoxyphenyl)-6-fluoro-1-benzothiophene-2-carboxylic acid, ATROP-2;
3-(3-chloro-2,4,5-trifluorophenyl)-6-fluoro-1-benzothiophene-2-carboxylic acid;
3-(4-chloro-2,6-difluoro-3-methoxyphenyl)-6-fluoro-1-benzothiophene-2-carboxylic acid;
6-chloro-3-(2,4,6-trifluoro-3-methoxyphenyl)-1-benzothiophene-2-carboxylic acid;
6-chloro-3-(3-ethyl-2,4,5-trifluorophenyl)-1-benzothiophene-2-carboxylic acid; or ammonium 3-(3-ethyl-2,4,5-trifluorophenyl)-6-fluoro-1-benzothiophene-2-carboxylate;
   or a pharmaceutically acceptable salt thereof.

In some embodiments, a compound of this invention may be co-administered with one or more anti-diabetic agents. Suitable anti-diabetic agents include insulin, metformin, GLP-1 receptor agonists (described herein above), an acetyl-CoA carboxylase (ACC) inhibitor (described herein above), SGLT2 inhibitors (described herein above), monoacylglycerol O-acyltransferase inhibitors, phosphodiesterase (PDE)-10 inhibitors, AMPK activators [e.g., ETC-1002 (bempedoic acid)], sulfonylureas (e.g., acetohexamide, chlorpropamide, diabinese, glibenclamide, glipizide, glyburide, glimepiride, gliclazide, glipentide, gliquidone, glisolamide, tolazamide, and tolbutamide), meglitinides, α-amylase inhibitors (e.g., tendamistat, trestatin and AL-3688), an α-glucoside hydrolase inhibitor (e.g., acarbose), α-glucosidase inhibitors (e.g., adiposine, camiglibose, emiglitate, miglitol, voglibose, pradimicin-Q, and salbostatin), PPARγ agonists (e.g., balaglitazone, ciglitazone, darglitazone, englitazone, isaglitazone, pioglitazone and rosiglitazone), PPAR α/γ agonists (e.g., CLX-0940, GW-1536, GW-1929, GW-2433, KRP-297, L-796449, LR-90, MK-0767 and SB-219994), protein tyrosine phosphatase-1B (PTP-1B) inhibitors [e.g., trodusquemine, hyrtiosal extract, and compounds disclosed by Zhang, S. et al., Drug Discovery Today, 12(9/10), 373-381 (2007)], SIRT-1 activators (e.g., resveratrol, GSK2245840 or GSK184072), dipeptidyl peptidase IV (DPP-IV) inhibitors (e.g., those in WO2005116014, sitagliptin, vildagliptin, alogliptin, dutogliptin, linagliptin and saxagliptin), insulin secretagogues, fatty acid oxidation inhibitors, A2 antagonists, c-jun amino-terminal kinase (JNK) inhibitors, glucokinase activators (GKa) such as those described in WO2010103437, WO2010103438, WO2010013161, WO2007122482, TTP-399, TTP-355, TTP-547, AZD1656, ARRY403, MK-0599, TAK-329, AZD5658 or GKM-001, insulin, insulin mimetics, glycogen phosphorylase inhibitors (e.g., GSK1362885), VPAC2 receptor agonists, glucagon receptor modulators such as those described in Demong, D. E. et al., Annual Reports in Medicinal Chemistry 2008, 43, 119-137, GPR119 modulators, particularly agonists, such as those described in WO2010140092, WO2010128425, WO2010128414, WO2010106457, Jones, R. M. et al., Annual Reports in Medicinal Chemistry 2009, 44, 149-170 (e.g., MBX-2982, GSK1292263, APD597 and PSN821), FGF21 derivatives or analogs such as those described in Kharitonenkov, A. et al., Current Opinion in Investigational Drugs 2009, 10(4)359-364, TGR5 (also termed GPBAR1) receptor modulators, particularly agonists, such as those described in Zhong, M., Current Topics in Medicinal Chemistry, 2010, 10(4), 386-396 and INT777, GPR40 agonists, such as those described in Medina, J. C., Annual Reports in Medicinal Chemistry, 2008, 43, 75-85, including but not limited to TAK-875, GPR120 modulators, particularly agonists, high-affinity nicotinic acid receptor (HM74A) activators, and SGLT1 inhibitors, such as GSK1614235. A further representative listing of anti-diabetic agents that can be combined with the compounds of the present invention can be found, for example, at page 28, line 35 through page 30, line 19 of WO2011005611.

Other antidiabetic agents could include inhibitors or modulators of carnitine palmitoyl transferase enzymes, inhibitors of fructose 1,6-diphosphatase, inhibitors of aldose reductase, mineralocorticoid receptor inhibitors, inhibitors of TORC2, inhibitors of CCR2 and/or CCR5, inhibitors of PKC isoforms (e.g., PKCα, PKCβ, PKCγ), inhibitors of fatty acid synthetase, inhibitors of serine palmitoyl transferase, modulators of GPR81, GPR39, GPR43, GPR41, GPR105, Kv1.3, retinol binding protein 4, glucocorticoid receptor, somatostain receptors (e.g., SSTR1, SSTR2, SSTR3 and SSTR5), inhibitors or modulators of PDHK2 or PDHK4, inhibitors of MAP4K4, modulators of IL1 family including IL1beta, and modulators of RXRalpha. In addition suitable anti-diabetic agents include mechanisms listed by Carpino, P. A., Goodwin, B. Expert Opin. Ther. Pat., 2010, 20(12), 1627-51.

The compounds of the present invention may be co-administered with anti-heart failure agents such as ACE inhibitors (e.g., captopril, enalapril, fosinopril, lisinopril, perindopril, quinapril, ramipril, trandolapril), Angiotensin II receptor blockers (e.g., candesartan, losartan, valsartan), Angiotensin-receptor neprilysin inhibitors (sacubitril/valsartan), I_{f} channel blocker Ivabradine, Beta-Adrenergic blocking agents (e.g., bisoprolol, metoprolol succinate, carvedilol), Aldosterone antagonists (e.g., spironolactone, eplerenone), hydralazine and isosorbide dinitrate, diuretics (e.g., furosemide, bumetanide, torsemide, chlorothiazide, amiloride, hydrochlorothiazide, Indapamide, Metolazone, Triamterene), or digoxin.

The compounds of the present invention may also be co-administered with cholesterol or lipid lowering agents including the following exemplary agents: HMG CoA reductase inhibitors (e.g., pravastatin, pitavastatin, lovastatin, atorvastatin, simvastatin, fluvastatin, NK-104 (a.k.a. itavastatin, or nisvastatin or nisbastatin) and ZD-4522 (a.k.a. rosuvastatin, or atavastatin or visastatin); squalene synthetase inhibitors; fibrates (e.g., gemfibrozil, pemafibrate, fenofibrate, clofibrate); bile acid sequestrants (such as questran, colestipol, colesevelam); ACAT inhibitors; MTP inhibitors; lipooxygenase inhibitors; cholesterol absorption inhibitors (e.g., ezetimibe); nicotinic acid agents (e.g., niacin, niacor, slo-niacin); omega-3 fatty acids (e.g., epanova, fish oil, eicosapentaenoic acid); cholesteryl ester transfer protein inhibitors (e.g., obicetrapib) and PCSK9 modulators [e.g., alirocumab, evolocumab, bococizumab, ALN-PCS (inclisiran)].

The compounds of the present invention may also be used in combination with antihypertensive agents and such antihypertensive activity is readily determined by those skilled in the art according to standard assays (e.g., blood pressure measurements). Examples of suitable anti-hypertensive agents include: alpha-adrenergic blockers; beta-adrenergic blockers; calcium channel blockers (e.g., diltiazem, verapamil, nifedipine and amlodipine); vasodilators (e.g., hydralazine), diruetics (e.g., chlorothiazide, hydrochlorothiazide, flumethiazide, hydroflumethiazide, bendroflumethiazide, methylchlorothiazide, trichloromethiazide, polythiazide, benzthiazide, ethacrynic acid tricrynafen, chlorthalidone, torsemide, furosemide, musolimine, bumetanide, triamtrenene, amiloride, spironolactone); renin inhibitors; ACE inhibitors (e.g., captopril, zofenopril, fosinopril, enalapril, ceranopril, cilazopril, delapril, pentopril, quinapril, ramipril, lisinopril); AT-1 receptor antagonists (e.g., losartan, irbesartan, valsartan); ET receptor antagonists (e.g., sitaxsentan, atrsentan and compounds disclosed in U.S. Pat. Nos. 5,612,359 and 6,043,265); Dual ET/AII antagonist (e.g., compounds disclosed in WO 00/01389); neutral endopeptidase (NEP) inhibitors; vasopepsidase inhibitors (dual NEP-ACE inhibitors) (e.g., gemopatrilat and nitrates). An exemplary antianginal agent is ivabradine.

Examples of suitable calcium channel blockers (L-type or T-type) include diltiazem, verapamil, nifedipine and amlodipine and mybefradil.

Examples of suitable cardiac glycosides include digitalis and ouabain.

In one embodiment, a compound of invention may be co-administered with one or more diuretics. Examples of suitable diuretics include (a) loop diuretics such as furosemide (such as LASIX™), torsemide (such as DEMADEX™), bemetanide (such as BUMEX™), and ethacrynic acid (such as EDECRIN™); (b) thiazide-type diuretics such as chlorothiazide (such as DIURIL™ ESIDRIX™ or HYDRODIURIL™), hydrochlorothiazide (such as MICROZIDE™ or ORETIC™) benzthiazide, hydroflumethiazide (such as SALURON™), bendroflumethiazide, methychlorthiazide, polythiazide, trichlormethiazide, and indapamide (such as LOZOL™); (c) phthalimidine-type diuretics such as chlorthalidone (such as HYGROTON™), and metolazone (such as ZAROXOLYN™); (d) quinazoline-type diuretics such as quinethazone; and (e) potassium-sparing diuretics such as triamterene (such as DYRENIUM™), and amiloride (such as MIDAMOR™ or MODURETIC™).

In another embodiment, a compound of the invention may be co-administered with a loop diuretic. In still another embodiment, the loop diuretic is selected from furosemide and torsemide. In still another embodiment, one or more compounds of Formula I or their pharmaceutically acceptable salts may be co-administered with furosemide. In still another embodiment, one or more compounds of Formula I or their pharmaceutically acceptable salts may be co-administered with torsemide which may optionally be a controlled or modified release form of torsemide.

In another embodiment, a compound of the invention may be co-administered with a thiazide-type diuretic. In still another embodiment, the thiazide-type diuretic is selected from the group consisting of chlorothiazide and hydrochlorothiazide. In still another embodiment, one or more compounds of Formula I or their pharmaceutically acceptable salts may be co-administered with chlorothiazide. In still another embodiment, one or more compounds of Formula I or their pharmaceutically acceptable salts may be co-administered with hydrochlorothiazide.

In another embodiment, one or more compounds of Formula I or their pharmaceutically acceptable salts may be co-administered with a phthalimidine-type diuretic. In still another embodiment, the phthalimidine-type diuretic is chlorthalidone.

Examples of suitable mineralocorticoid receptor antagonists include sprionolactone and eplerenone.

Examples of suitable phosphodiesterase inhibitors include: PDE III inhibitors (such as cilostazol); and PDE V inhibitors (such as sildenafil).

Those skilled in the art will recognize that the compounds of this invention may also be used in conjunction with other cardiovascular or cerebrovascular treatments including Percutaneous Coronary Intervention (PCI), stenting, drug-eluting stents, stem cell therapy and medical devices such as implanted pacemakers, defibrillators, or cardiac resynchronization therapy.

Particularly when provided as a single dosage unit, the potential exists for a chemical interaction between the combined active ingredients. For this reason, when a compound of this invention and a second therapeutic agent are combined in a single dosage unit they may be formulated such that although the active ingredients are combined in a single dosage unit, the physical contact between the active ingredients is minimized (that is, reduced). For example, one active ingredient may be enteric-coated. By enteric-coating one of the active ingredients, it is possible not only to minimize the contact between the combined active ingredients, but also, it is possible to control the release of one of these components in the gastrointestinal tract such that one of these components is not released in the stomach but rather is released in the intestines. One of the active ingredients may also be coated with a material that effects a sustained release throughout the gastrointestinal tract and also serves to minimize physical contact between the combined active ingredients. Furthermore, the sustained-released component can be additionally enteric-coated such that the release of this component occurs only in the intestine. Still another approach would involve the formulation of a combination product in which the one component is coated with a sustained and/or enteric-release polymer, and the other component is also coated with a polymer such as a low viscosity grade of hydroxypropyl methylcellulose (HPMC) or other appropriate materials as known in the art, in order to further separate the active components. The polymer coating serves to form an additional barrier to interaction with the other component.

These as well as other ways of minimizing contact between the components of combination products of the present invention, whether administered in a single dosage form or administered in separate forms but at the same time by the same manner, will be readily apparent to those skilled in the art, once armed with the present disclosure.

Another approach may involve the formulation of a combination product in which both active components are combined with a material that effects a sustained release throughout the gastrointestinal tract of both active ingredients.

In some embodiments of combination therapy treatment, both the compounds of this invention and the other drug therapies are administered to patients such as mammals (e.g., humans, male or female) by conventional methods.

### Kits

Another aspect of the invention provides kits comprising the compound of the invention or pharmaceutical compositions comprising the compound of the invention. A kit may include, in addition to the compound of the invention or pharmaceutical composition thereof, diagnostic or therapeutic agents. A kit may also include instructions for use in a diagnostic or therapeutic method. In some embodiments, the kit includes the compound or a pharmaceutical composition thereof and a diagnostic agent. In other embodiments, the kit includes the compound or a pharmaceutical composition thereof and one or more therapeutic agents as described in the co-administration section hereinabove.

In yet another embodiment, the invention comprises kits that are suitable for use in performing the methods of treatment described herein. In one embodiment, the kit contains a first dosage form comprising one or more of the compounds of the invention in quantities sufficient to carry out the methods of the invention. In another embodiment, the kit comprises one or more compounds of the invention in quantities sufficient to carry out the methods of the invention and a container for the dosage and a container for the dosage.

### EXAMPLES

The following illustrate the synthesis of various compounds of the present invention. Additional compounds within the scope of this invention may be prepared using the methods illustrated in these Examples, either alone or in combination with techniques generally known in the art. All starting materials in these Preparations and Examples are either commercially available or can be prepared by methods known in the art or as described herein.

Reactions were performed in air or, when oxygen- or moisture-sensitive reagents or intermediates were employed, under an inert atmosphere (nitrogen or argon). When appropriate, reaction apparatuses were dried under dynamic vacuum using a heat gun, and anhydrous solvents (Sure-Seal™ products from Aldrich Chemical Company, Milwaukee, Wisconsin or DriSolv™ products from EMD Chemicals, Gibbstown, NJ) were employed. In some cases, some commercial solvents may have been passed through columns packed with 4A molecular sieves, until the following QC standards for water were attained: a) <100 ppm for dichloromethane, toluene, N,N-dimethylformamide, and tetrahydrofuran; b) <180 ppm for methanol, ethanol, 1,4-dioxane, and diisopropylamine. For very sensitive reactions, some solvents may have been further treated with metallic sodium, calcium hydride, or molecular sieves, and distilled just prior to use. Other commercial solvents and reagents were used without further purification. For syntheses referencing procedures in other Examples or Methods, reaction conditions (reaction time and temperature) may vary. Products were generally dried under vacuum before being carried on to further reactions or submitted for biological testing.

When indicated, reactions were heated by microwave irradiation using Biotage Initiator or Personal Chemistry Emrys Optimizer microwave instruments. Reaction progress was monitored using thin-layer chromatography (TLC), liquid chromatography-mass spectrometry (LCMS), high-performance liquid chromatography (HPLC), and/or gas chromatography-mass spectrometry (GCMS) analyses. TLC was performed on pre-coated silica gel plates with a fluorescence indicator (254 nm excitation wavelength) and visualized under UV light and/or with I₂, KMnO₄, CoCl₂, phosphomolybdic acid, or ceric ammonium molybdate stains. LCMS data were acquired on an Agilent 1100 Series instrument with a Leap Technologies autosampler, Gemini C18 columns, acetonitrile/water gradients, and either trifluoroacetic acid, formic acid, or ammonium hydroxide modifiers. The column eluent was analyzed using a Waters ZQ mass spectrometer scanning in both positive and negative ion modes from 100 to 1200 Da. Other similar instruments were also used. HPLC data were generally acquired on an Agilent 1100 Series instrument using Gemini or XBridge C18 columns, acetonitrile/water gradients, and either trifluoroacetic acid or ammonium hydroxide modifiers. GCMS data were acquired using a Hewlett Packard 6890 oven with an HP 6890 injector, HP-1 column (12 m×0.2 mm×0.33 μm), and helium carrier gas. Samples were analyzed on an HP 5973 mass selective detector, scanning from 50 to 550 Da using electron ionization. Purifications were performed by medium performance liquid chromatography (MPLC) using Isco CombiFlash Companion, AnaLogix IntelliFlash 280, Biotage SP1, or Biotage Isolera One instruments and pre-packed Isco RediSep or Biotage Snap silica cartridges. Chiral purifications were generally performed by chiral supercritical fluid chromatography (SFC) using Berger or Thar instruments; ChiralPAK-AD, -AS, —IC, Chiralcel-OD, or —OJ columns; and CO₂ mixtures with methanol, ethanol, propan-2-ol, or acetonitrile, alone or modified using trifluoroacetic acid or propan-2-amine. UV detection was used to trigger fraction collection. For syntheses referencing procedures in other Examples or Methods, purifications may vary: in general, solvents and the solvent ratios used for eluents/gradients were chosen to provide appropriate R_{f}s or retention times.

Mass spectrometry data are reported from LCMS analyses. Mass spectrometry (MS) was performed via atmospheric pressure chemical ionization (APCI), electrospray ionization (ESI), electron impact ionization (EI) or electron scatter (ES) ionization sources. Proton nuclear magnetic spectroscopy (¹H NMR) chemical shifts are given in parts per million downfield from tetramethylsilane and were recorded on 300, 400, 500, or 600 MHz Varian, Bruker, or Jeol spectrometers. Chemical shifts are expressed in parts per million (ppm, δ) referenced to the deuterated solvent residual peaks (chloroform, 7.26 ppm; CD₂HOD, 3.31 ppm; acetonitrile-d₂, 1.94 ppm; dimethyl sulfoxide-d₅, 2.50 ppm; DHO, 4.79 ppm). The peak shapes are described as follows: s, singlet; d, doublet; t, triplet; q, quartet; quin, quintet; m, multiplet; br s, broad singlet; app, apparent. Analytical SFC data were acquired on a Berger analytical instrument as described above. Optical rotation data were acquired on a PerkinElmer model 343 polarimeter using a 1 dm cell. Silica gel chromatography was performed primarily using medium-pressure Biotage or ISCO systems using columns pre-packaged by various commercial vendors including Biotage and ISCO. Microanalyses were performed by Quantitative Technologies Inc. and were within 0.4% of the calculated values.

Unless otherwise noted, chemical reactions were performed at room temperature (about 23 degrees Celsius).

Unless noted otherwise, all reactants were obtained commercially without further purifications or were prepared using methods known in the literature.

The terms “concentrated,” “evaporated,” and “concentrated in vacuo” refer to the removal of solvent at reduced pressure on a rotary evaporator with a bath temperature less than 60° C. The abbreviation “min” and “h” stand for “minutes” and “hours” respectively. The term “TLC” refers to thin-layer chromatography, “room temperature or ambient temperature” means a temperature between 18 and 25° C., “GCMS” refers to gas chromatography-mass spectrometry, “LCMS” refers to liquid chromatography-mass spectrometry, “UPLC” refers to ultra-performance liquid chromatography and “HPLC” refers to high-performance liquid chromatography, “SFC” refers to supercritical fluid chromatography.

Hydrogenation may be performed in a Parr Shaker under pressurized hydrogen gas, or in a Thales-nano H-Cube flow hydrogenation apparatus at full hydrogen and a flow rate between 1 and 2 mL/minute at the specified temperature.

HPLC, UPLC, LCMS, GCMS, and SFC retention times were measured using the methods noted in the procedures.

In some examples, chiral separations were carried out to separate enantiomers or diastereomers of certain compounds of the invention (in some examples, the separated enantiomers are designated as ENANT-1 and ENANT-2, according to their order of elution; similarly, separated diastereomers are designated as DIAST-1 and DIAST-2, according to their order of elution). In some examples, the optical rotation of an enantiomer was measured using a polarimeter. According to its observed rotation data (or its specific rotation data), an enantiomer with a clockwise rotation was designated as the (+)-enantiomer and an enantiomer with a counter-clockwise rotation was designated as the (−)-enantiomer. Racemic compounds are indicated either by the absence of drawn or described stereochemistry, or by the presence of (+/−) adjacent to the structure; in this latter case, the indicated stereochemistry represents just one of the two enantiomers that make up the racemic mixture.

The compounds and intermediates described below were named using the naming convention provided with ACD/ChemSketch 2020.2.1.1, File Version C25H41, Build 121153 (Advanced Chemistry Development, Inc., Toronto, Ontario, Canada). The naming convention provided with ACD/ChemSketch 2020.2.1.1 is well known by those skilled in the art and it is believed that the naming convention provided with ACD/ChemSketch 2020.2.1.1 generally comports with the IUPAC (International Union for Pure and Applied Chemistry) recommendations on Nomenclature of Organic Chemistry and the CAS Index rules.

### Example 1

### Ammonium 5-{4-[(1-{[4-(propan-2-yl)phenyl]carbamoyl}-D-prolyl)amino]phenyl}pyridine-2-carboxylate (1)

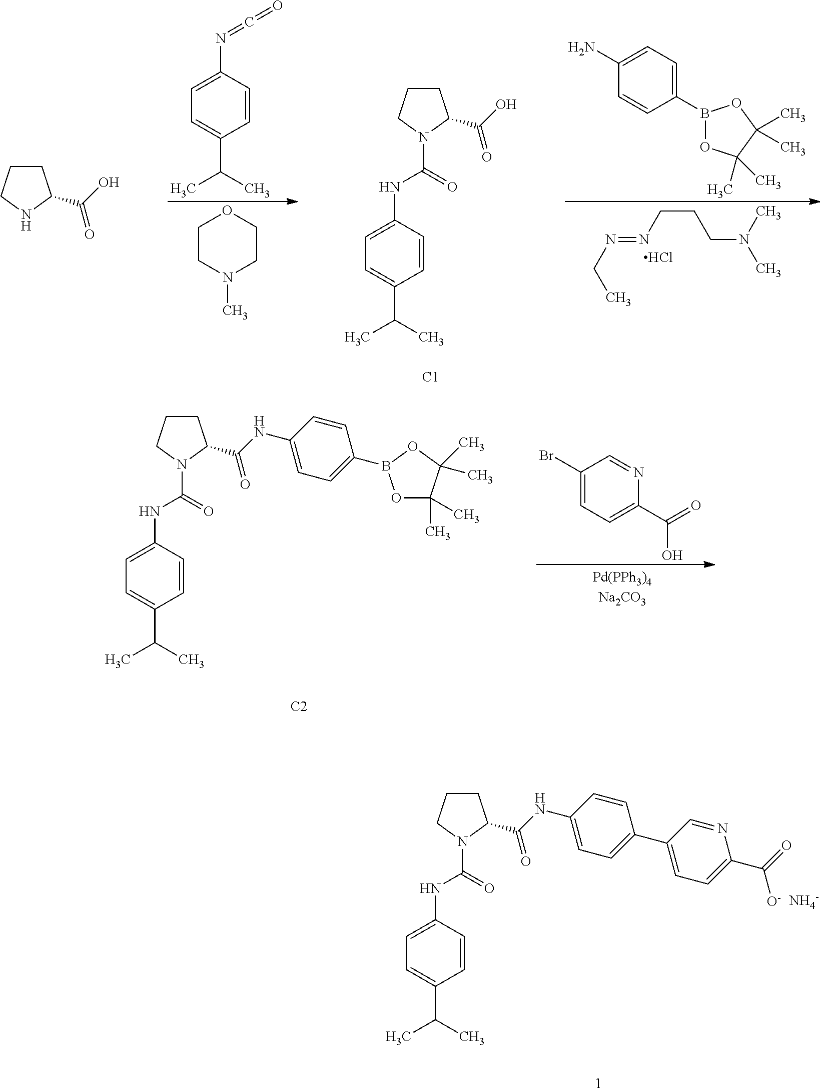

### Step 1. Synthesis of 1-{[4-(propan-2-yl)phenyl]carbamoyl}-D-proline (C1)

4-Methylmorpholine (20.5 mL, 186 mmol) was added to a 2° C. to 3° C. mixture of D-proline (21.4 g, 186 mmol) in tetrahydrofuran (520 mL). After 2 minutes, 1-isocyanato-4-(propan-2-yl)benzene (25.0 g, 155 mmol) was added over 30 seconds, whereupon stirring was continued for 5 minutes before the reaction mixture was removed from the ice bath and allowed to stir at room temperature. Two hours later, LCMS analysis indicated formation of C1: LCMS m/z 277.4 [M+H]⁺. Water (500 mL) was added, followed by solid sodium bicarbonate (19.5 g, 233 mmol), yielding a pH of 7 to 8. The resulting mixture was washed with methyl tert-butyl ether (2×600 mL); the aqueous layer was then acidified to pH 2 by addition of concentrated hydrochloric acid and stirred for 20 minutes. Filtration, followed by rinsing of the filter cake with water, provided C1 as a white solid. Yield: 39.1 g, 141 mmol, 91%. ¹H NMR (400 MHz, DMSO-d₆) δ 12.36 (br s, 1H), 8.16 (s, 1H), 7.38 (d, J=8.6 Hz, 2H), 7.09 (d, J=8.5 Hz, 2H), 4.34-4.26 (m, 1H), 3.58-3.50 (m, 1H), 3.49-3.41 (m, 1H), 2.81 (septet, J=6.9 Hz, 1H), 2.22-2.11 (m, 1H), 1.97-1.83 (m, 3H), 1.17 (d, J=6.9 Hz, 6H).

### Step 2. Synthesis of (2R)—N¹-[4-(propan-2-yl)phenyl]-N²-[4-(4,4,5,5-tetramethyl-1,3,2-dioxaborolan-2-yl)phenyl]pyrrolidine-1,2-dicarboxamide (C2)

A solution of C1 (3.00 g, 10.9 mmol), 4-(4,4,5,5-tetramethyl-1,3,2-dioxaborolan-2-yl)aniline (2.38 g, 10.9 mmol), and 1-[3-(dimethylamino)propyl]-3-ethylcarbodiimide hydrochloride (2.71 g, 14.1 mmol) in dichloromethane (54 mL) was stirred at room temperature overnight. The reaction mixture was then diluted with dichloromethane, washed sequentially with water and saturated aqueous sodium chloride solution, dried over sodium sulfate, filtered, and concentrated in vacuo. Trituration with diethyl ether provided C2 as an off-white solid. Yield: 4.37 g, 9.15 mmol, 84%. LCMS m/z 478.4 [M+H]⁺. ¹H NMR (400 MHz, DMSO-d₆) δ 10.09 (s, 1H), 8.18 (s, 1H), 7.61 (AB quartet, J_{AB}=8.7 Hz, Δv_{AB}=10.3 Hz, 4H), 7.39 (d, J=8.6 Hz, 2H), 7.08 (d, J=8.6 Hz, 2H), 4.45 (dd, J=8.2, 3.6 Hz, 1H), 3.68-3.59 (m, 1H), 3.54-3.45 (m, 1H), 2.80 (septet, J=6.9 Hz, 1H), 2.23-2.10 (m, 1H), 2.08-1.86 (m, 3H), 1.28 (s, 12H), 1.16 (d, J=6.9 Hz, 6H).

### Step 3. Synthesis of ammonium 5-{4-[(1-{[4-(propan-2-yl)phenyl]carbamoyl}-D-prolyl)amino]phenyl}pyridine-2-carboxylate (1)

A mixture of C2 (125 mg, 0.262 mmol), 5-bromopyridine-2-carboxylic acid (63.5 mg, 0.314 mmol), tetrakis(triphenylphosphine)palladium(0) (15.1 mg, 13.1 μmol), and sodium carbonate (98%. 142 mg, 1.31 mmol) in a mixture of 1,2-dimethoxyethane (1.25 mL), water (0.5 mL), and ethanol (0.1 mL) was heated at 90° C. overnight, whereupon the reaction mixture was added to 1 M hydrochloric acid. The resulting mixture was extracted three times with ethyl acetate; during the extractions, insoluble material, a white solid, was collected via filtration (75 mg). The ethyl acetate filtrate was washed with saturated aqueous sodium chloride solution, dried over sodium sulfate, filtered, and concentrated in vacuo, providing a light-yellow solid (55 mg). These two batches of solid were purified via reversed-phase HPLC (Column: Waters XBridge C18, 19×100 mm, 5 μm; Mobile phase A: water containing 0.03% ammonium hydroxide; Mobile phase B: acetonitrile containing 0.03% ammonium hydroxide; Gradient: 5% to 95% B; Flow rate: 25 mL/minute), affording ammonium 5-{4-[(1-{[4-(propan-2-yl)phenyl]carbamoyl}-D-prolyl)amino]phenyl}pyridine-2-carboxylate (1). Combined yield: 10.7 mg, 21.8 μmol, 8%. LCMS m/z 473.4 [M+H]⁺. ¹H NMR (600 MHz, DMSO-d₆), characteristic peaks: δ 10.20 (s, 1H), 9.02-8.99 (m, 1H), 8.23 (dd, J=8.2, 2.4 Hz, 1H), 8.20 (s, 1H), 8.08 (d, J=8.2 Hz, 1H), 7.78 (AB quartet, J_{AB}=9.0 Hz, Δv_{AB}=9.7 Hz, 4H), 7.39 (br d, J=8.6 Hz, 2H), 7.08 (d, J=8.6 Hz, 2H), 4.47 (dd, J=8.3, 3.8 Hz, 1H), 3.67-3.61 (m, 1H, assumed; partially obscured by water peak), 2.80 (septet, J=6.9 Hz, 1H), 2.23-2.15 (m, 1H), 2.07-2.00 (m, 1H), 2.00-1.89 (m, 2H), 1.16 (d, J=6.8 Hz, 6H).

### Alternate Synthesis of Example 1 (methanesulfonate salt)

### 5-{4-[(1-{[4-(Propan-2-yl)phenyl]carbamoyl}-D-prolyl)amino]phenyl}pyridine-2-carboxylic acid, methanesulfonate salt (1, methanesulfonate salt)

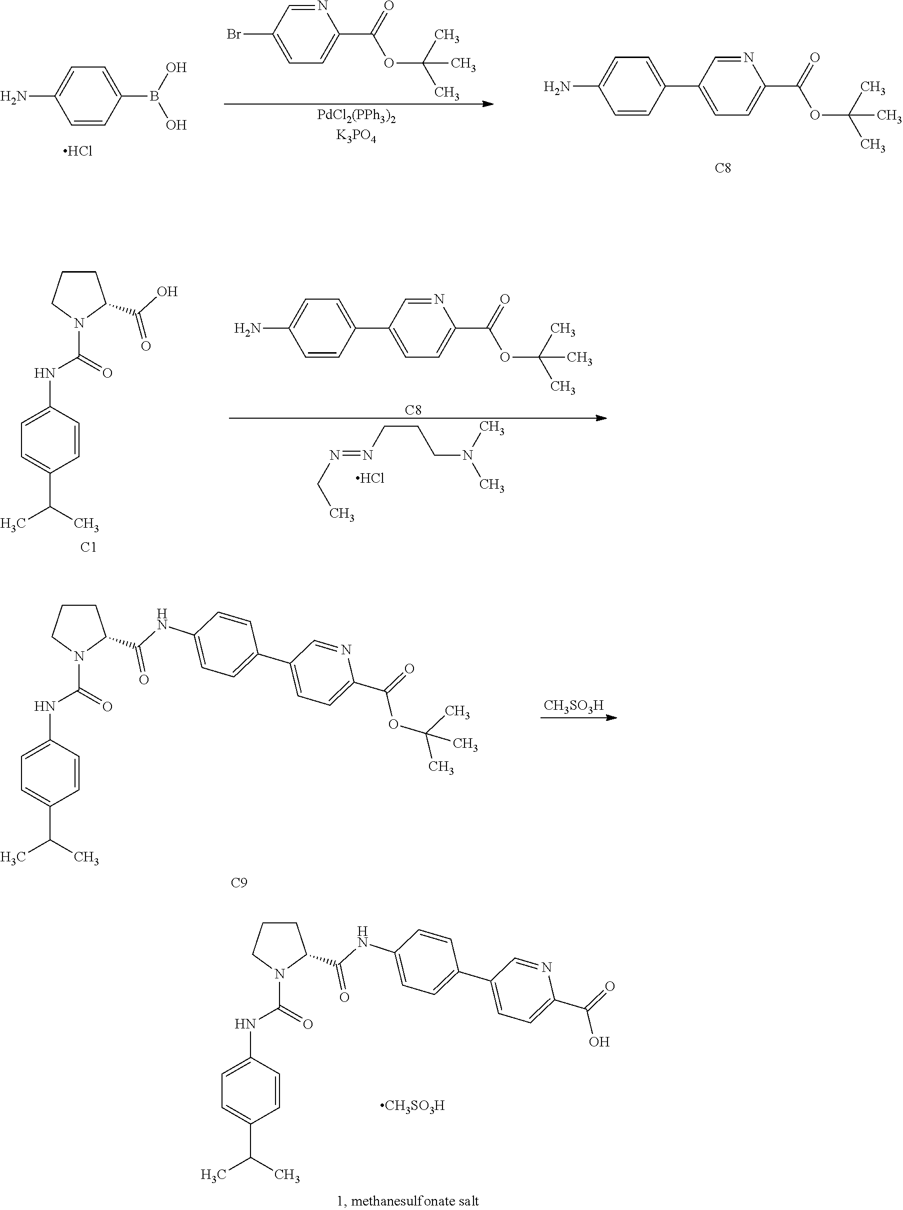

### Step 1. Synthesis of tert-butyl 5-(4-aminophenyl)pyridine-2-carboxylate (C8)

A mixture of tert-butyl 5-bromopyridine-2-carboxylate (1.06 g, 4.11 mmol), (4-aminophenyl)boronic acid, hydrochloride salt (710 mg, 4.09 mmol), and bis(triphenylphosphine)palladium(II) dichloride (287 mg, 0.409 mmol) in 1,4-dioxane (30 mL) was sparged with nitrogen for 10 minutes and then gradually heated to 50° C. Simultaneously, an aqueous solution of tripotassium phosphate (1.5 M; 8.19 mL, 12.3 mmol) was prepared and sparged with nitrogen for 10 minutes. The aqueous base solution was added to the reaction mixture, which was then gradually heated to 90° C. and stirred at 90° C. overnight. After cooling to room temperature, the reaction mixture was added to a mixture of water (300 mL) and saturated aqueous sodium chloride solution (100 mL); the resulting suspension was extracted with ethyl acetate (2×250 mL) and then with a solution of methanol in dichloromethane (10%, 2×150 mL). The combined organic layers were concentrated in vacuo and purified via silica gel chromatography (Gradient: 0% to 100% ethyl acetate in heptane). The product-containing fractions were dried over sodium sulfate, filtered, and concentrated under reduced pressure to provide C8 as a solid. Yield: 736 mg, 2.72 mmol, 66%. LCMS m/z 271.2 [M+H]⁺. ¹H NMR (400 MHz, DMSO-d₆) δ 8.90 (br d, J=2 Hz, 1H), 8.07 (dd, component of ABX system, J=8.3, 2.4 Hz, 1H), 7.95 (br d, half of AB quartet, J=8.2 Hz, 1H), 7.51 (d, J=8.6 Hz, 2H), 6.69 (d, J=8.6 Hz, 2H), 5.51 (br s, 2H), 1.56 (s, 9H).

### Step 2. Synthesis of tert-butyl 5-{4-[(1-{[4-(propan-2-yl)phenyl]carbamoyl}-D-prolyl)amino]phenyl}pyridine-2-carboxylate (C9)

1-[3-(Dimethylamino)propyl]-3-ethylcarbodiimide hydrochloride (98%, 1.10 g, 5.62 mmol) was added to a solution of C1 (1.29 g, 4.67 mmol) and C8 (1.26 g, 4.66 mmol) in N,N-dimethylacetamide (20 mL). After the reaction mixture had been stirred overnight at room temperature, it was added to a mixture of water (250 mL) and saturated aqueous sodium chloride solution (50 mL). The resulting mixture was extracted with ethyl acetate (2×200 mL) and then with methanol in dichloromethane (10%, 2×100 mL), and the combined organic layers were dried over sodium sulfate, filtered, and concentrated in vacuo, affording C9 as a solid. Yield: 785 mg, 1.48 mmol, 32%. LCMS m/z 529.3 [M+H]⁺. ¹H NMR (400 MHz, DMSO-d₆) δ 10.19 (s, 1H), 9.01 (br d, J=2 Hz, 1H), 8.24-8.18 (m, 2H), 8.03 (br d, J=8.2 Hz, 1H), 7.78 (s, 4H), 7.40 (d, J=8.6 Hz, 2H), 7.09 (d, J=8.6 Hz, 2H), 4.48 (dd, J=8.3, 3.7 Hz, 1H), 3.70-3.60 (m, 1H), 3.57-3.46 (m, 1H), 2.80 (septet, J=6.9 Hz, 1H), 2.26-2.13 (m, 1H), 2.10-1.88 (m, 3H), 1.57 (s, 9H), 1.16 (d, J=6.9 Hz, 6H).

### Step 3. Synthesis of 5-{4-[(1-{[4-(propan-2-yl)phenyl]carbamoyl}-D-prolyl)amino]phenyl}pyridine-2-carboxylic acid, methanesulfonate salt (1, methanesulfonate salt)

Methanesulfonic acid (0.198 mL, 3.05 mmol) was added to a solution of C9 (785 mg, 1.48 mmol) in 1,1,1,3,3,3-hexafluoropropan-2-ol (7.8 mL). After the reaction mixture had been stirred overnight, it was concentrated under reduced pressure, and the residue was triturated with diethyl ether, then with water (150 mL) and diethyl ether (150 mL). The resulting solid was milled in methyl tert-butyl ether, filtered, and dried, whereupon it was triturated with ethyl acetate to afford 5-{4-[(1-{[4-(propan-2-yl)phenyl]carbamoyl}-D-prolyl)amino]phenyl}pyridine-2-carboxylic acid, methanesulfonate salt (1, methanesulfonate salt) as a pale-yellow solid. Yield: 690 mg, 1.21 mmol, 82%. LCMS m/z 473.3 [M+H]⁺. ¹H NMR (400 MHz, DMSO-d₆) δ 10.20 (s, 1H), 9.02 (br d, J=2 Hz, 1H), 8.27 (dd, J=8.2, 2.4 Hz, 1H), 8.20 (br s, 1H), 8.10 (br d, J=8.2 Hz, 1H), 7.79 (AB quartet, J_{AB}=9.2 Hz, Δv_{AB}=7.5 Hz, 4H), 7.40 (d, J=8.6 Hz, 2H), 7.09 (d, J=8.6 Hz, 2H), 4.48 (dd, J=8.3, 3.7 Hz, 1H), 3.70-3.61 (m, 1H), 3.56-3.47 (m, 1H), 2.80 (septet, J=6.9 Hz, 1H), 2.34 (s, 3H), 2.26-2.14 (m, 1H), 2.10-1.88 (m, 3H), 1.16 (d, J=6.9 Hz, 6H).

### Example 2

### 6-{4-[(1-{[4-(Propan-2-yl)phenyl]carbamoyl}-D-prolyl)amino]phenyl}pyridine-3-carboxylic acid (2)

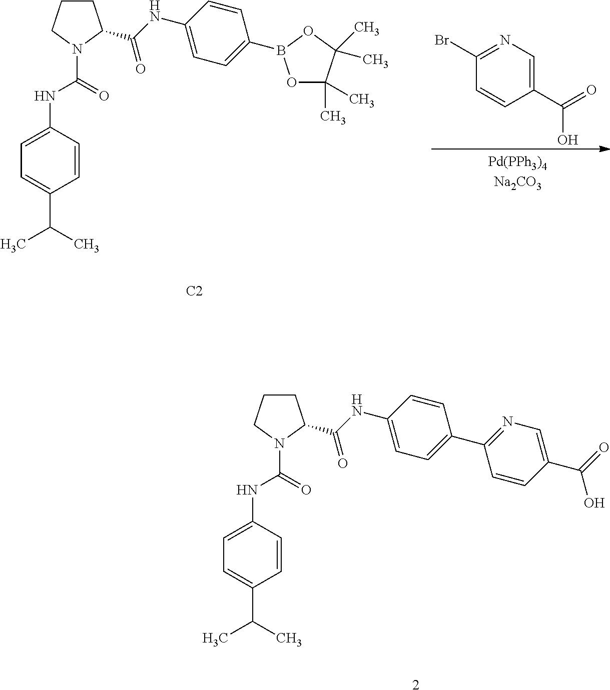 [SMILES: CC(C)c1ccc(NC(=O)N2CCC[C@@H]2C(=O)Nc2ccc(B3OC(C)(C)C(C)(C)O3)cc2)cc1 ; O=C(O)c1ccc(Br)nc1 ; "C2" ; CC(C)c1ccc(NC(=O)N2CCC[C@@H]2C(=O)Nc2ccc(-c3ccc(C(=O)O)cn3)cc2)cc1 ; "2"]

A mixture of C2 (240 mg, 0.503 mmol), 6-bromopyridine-3-carboxylic acid (122 mg, 0.604 mmol), tetrakis(triphenylphosphine)palladium(0) (29.0 mg, 25.1 μmol), and sodium carbonate (98%, 272 mg, 2.51 mmol) in a mixture of 1,2-dimethoxyethane (2.5 mL), water (1 mL), and ethanol (0.2 mL) was heated 90° C. for 4 hours, and then stirred at room temperature for 3 days. After the reaction mixture had been added to 1 M hydrochloric acid, the resulting mixture was extracted three times with ethyl acetate; the combined organic layers were washed with saturated aqueous sodium chloride solution, dried over sodium sulfate, filtered, and concentrated in vacuo. The residue was triturated with diethyl ether to provide a solid, which was purified via reversed-phase HPLC (Column: Waters Sunfire C18, 19×100 mm, 5 μm; Mobile phase A: 0.05% trifluoroacetic acid in water (v/v); Mobile phase B: 0.05% trifluoroacetic acid in acetonitrile (v/v); Gradient: 5% to 95% B over 8.54 minutes, followed by 95% B for 1.46 minutes; Flow rate: 25 mL/minute), affording 6-{4-[(1-{[4-propan-2-yl)phenyl]carbamoyl}-D-prolyl)amino]phenyl}pyridine-3-carboxylic acid (2). Yield: 58.0 mg, 0.123 mmol, 24%. LCMS m/z 473.4 [M+H]⁺. ¹H NMR (400 MHz, DMSO-d₆) δ 10.22 (s, 1H), 9.10 (br d, J=2.2 Hz, 1H), 8.28 (dd, J=8.4, 2.3 Hz, 1H), 8.20 (s, 1H), 8.13 (d, J=8.8 Hz, 2H), 8.05 (br d, J=8.4 Hz, 1H), 7.77 (d, J=8.8 Hz, 2H), 7.39 (d, J=8.6 Hz, 2H), 7.08 (d, J=8.6 Hz, 2H), 4.48 (dd, J=8.3, 3.7 Hz, 1H), 3.64 (ddd, J=9.3, 7.6, 4.7 Hz, 1H), 3.54-3.48 (m, 1H, assumed; partially obscured by water peak), 2.80 (septet, J=6.9 Hz, 1H), 2.23-2.15 (m, 1H), 2.08-1.99 (m, 1H), 1.99-1.90 (m, 2H), 1.16 (d, J=6.9 Hz, 6H).

### Example 3

### 4′-[(1-{[4-(Propan-2-yl)phenyl]carbamoyl}-D-prolyl)amino][1,1′-biphenyl]-3-carboxylic acid (3)

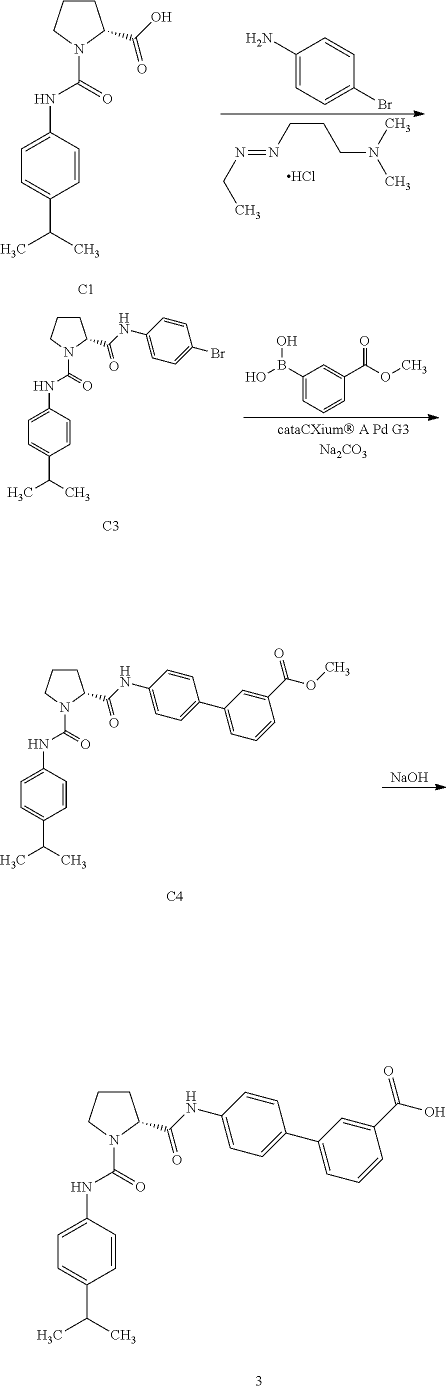

### Step 1. Synthesis of (2R)—N²-(4-bromophenyl)-N¹-[4-(propan-2-yl)phenyl]pyrrolidine-1,2-dicarboxamide (C3)

A mixture of 4-bromoaniline (1.94 g, 11.3 mmol), C1 (3.12 g, 11.3 mmol), and 1-[3-(dimethylamino)propyl]-3-ethylcarbodiimide hydrochloride (2.60 g, 13.6 mmol) in dichloromethane (50 mL) was stirred at room temperature for 16 hours, whereupon LCMS analysis indicated conversion to C3: LCMS m/z 430.2 (bromine isotope pattern observed) [M+H]⁺. The reaction mixture was poured into water and extracted with dichloromethane; the organic layer was dried over magnesium sulfate, filtered, and concentrated in vacuo to afford C3 as a white solid. Yield: 4.82 g, 11.2 mmol, 99%. ¹H NMR (400 MHz, chloroform-d) δ 9.94 (br s, 1H), 7.41 (AB quartet, J_{AB}=8.9 Hz, Δv_{AB}=27.9 Hz, 4H), 7.25 (AB quartet, J_{AB}=8.5 Hz, Δv_{AB}=46.6 Hz, 4H), 6.30 (br s, 1H), 4.74 (br d, J=8.0 Hz, 1H), 3.55 (ddd, J=8, 8, 1.9 Hz, 1H), 3.44-3.36 (m, 1H), 2.89 (septet, J=7.0 Hz, 1H), 2.64 (dd, J=12.6, 6.3 Hz, 1H), 2.29-2.07 (m, 2H), 1.89 (tdd, J=12.4, 8.0, 6.8 Hz, 1H), 1.24 (d, J=6.9 Hz, 6H).

### Step 2. Synthesis of methyl 4′-[(1-{[4-(propan-2-yl)phenyl]carbamoyl}-D-prolyl)amino][1,1′-biphenyl]-3-carboxylate (C4)

To a solution of C3 (30 mg, 70 μmol) and [3-(methoxycarbonyl)phenyl]boronic acid (15.3 mg, 85.0 μmol) in 1,4-dioxane (1.0 mL) was added an aqueous solution of sodium carbonate (2 M; 71.0 μL, 0.142 mmol), followed by mesylate[(di(1-adamantyl)-n-butylphosphine)-2-(2′-amino-1,1′-biphenyl)]palladium(II) (cataCXium® A Pd G3; 5.17 mg, 7.10 μmol). The reaction mixture was degassed for 5 minutes, whereupon it was heated at 90° C. overnight. After cooling to room temperature, one-third of the reaction mixture was partitioned between ethyl acetate (20 mL) and water (10 mL); the organic layer was washed sequentially with water (10 mL) and saturated aqueous sodium chloride solution, dried over magnesium sulfate, filtered, and concentrated in vacuo. Purification via reversed-phase HPLC (Column: Waters Sunfire C18, 19×100 mm, 5 μm; Mobile phase A: 0.05% trifluoroacetic acid in water (v/v); Mobile phase B: 0.05% trifluoroacetic acid in acetonitrile (v/v); Gradient: 45% to 85% B over 8.5 minutes, then 85% to 95% B over 0.5 minutes, then 95% B for 1.0 minute; Flow rate: 25 mL/minute), providing C4. Yield: 6.8 mg, 14 μmol, approximately 60%. LCMS m/z 486.5 [M+H]⁺. ¹H NMR (600 MHz, DMSO-d₆) δ 10.12 (s, 1H), 8.19 (s, 1H), 8.17 (br s, 1H), 7.93 (br d, J=7.9 Hz, 1H), 7.91 (br d, J=7.7 Hz, 1H), 7.70 (AB quartet, J_{AB}=8.6 Hz, Δv_{AB}=38.9 Hz, 4H), 7.60 (t, J=7.7 Hz, 1H), 7.39 (d, J=8.4 Hz, 2H), 7.08 (d, J=8.4 Hz, 2H), 4.47 (dd, J=8.3, 3.7 Hz, 1H), 3.88 (s, 3H), 3.68-3.61 (m, 1H), 3.54-3.47 (m, 1H, assumed; perturbed by adjacent water peak), 2.79 (septet, J=7.0 Hz, 1H), 2.23-2.14 (m, 1H), 2.08-2.00 (m, 1H), 2.00-1.88 (m, 2H), 1.16 (d, J=6.9 Hz, 6H).

The remaining two-thirds of the crude reaction mixture was advanced to the following step.

### Step 4. Synthesis of 4′-[(1-{[4-(propan-2-yl)phenyl]carbamoyl}-D-prolyl)amino][1,1′-biphenyl]-3-carboxylic acid (3)

The reaction mixture from the previous step, which contained C4 (547 μmol) was treated with aqueous sodium hydroxide solution (2 M; 71.0 μL, 0.142 mmol) and stirred at room temperature for 3 days. Aqueous sodium hydroxide solution (2 M; 71.0 μL, 0.142 mmol) was again added and stirring was continued at 50° C. for 18 hours, whereupon the reaction mixture was cooled to room temperature, and adjusted to a pH of approximately 3 by addition of 1 M hydrochloric acid. The resulting mixture was partitioned between ethyl acetate (20 mL) and water (10 mL), and the organic layer was washed sequentially with water (2×10 mL) and saturated aqueous sodium chloride solution, dried over magnesium sulfate, filtered, and concentrated in vacuo. Reversed-phase HPLC (Column: Waters Sunfire C18, 19×100 mm, 5 μm; Mobile phase A: 0.05% trifluoroacetic acid in water (v/v); Mobile phase B: 0.05% trifluoroacetic acid in acetonitrile (v/v); Gradient: 5% to 95% B over 8.54 minutes, followed by 95% B for 1.46 minutes; Flow rate: 25 mL/minute) afforded 4′-[(1-{[4-(propan-2-yl)phenyl]carbamoyl}-D-prolyl)amino][1,1′-biphenyl]-3-carboxylic acid (3). Yield: 4.3 mg, 9.1 μmol, approximately 19% over 2 steps. LCMS m/z 472.4 [M+H]⁺. ¹H NMR (600 MHz, DMSO-d₆), characteristic peaks: δ 10.11 (s, 1H), 8.19 (s, 1H), 8.15 (s, 1H), 7.92-7.85 (m, 2H), 7.69 (AB quartet, J_{AB}=8.3 Hz, Δv_{AB}=41.6 Hz, 4H), 7.59-7.52 (m, 1H), 7.39 (d, J=8.3 Hz, 2H), 7.08 (d, J=8.5 Hz, 2H), 4.47 (dd, J=8.3, 3.7 Hz, 1H), 3.68-3.61 (m, 1H), 2.84-2.76 (m, 1H), 2.23-2.14 (m, 1H), 2.08-2.00 (m, 1H), 2.00-1.89 (m, 2H), 1.16 (d, J=6.9 Hz, 6H).

### Alternate Synthesis of Example 3

### 4′-[(1-{[4-(Propan-2-yl)phenyl]carbamoyl}-D-prolyl)amino][1,1′-biphenyl]-3-carboxylic acid (3)

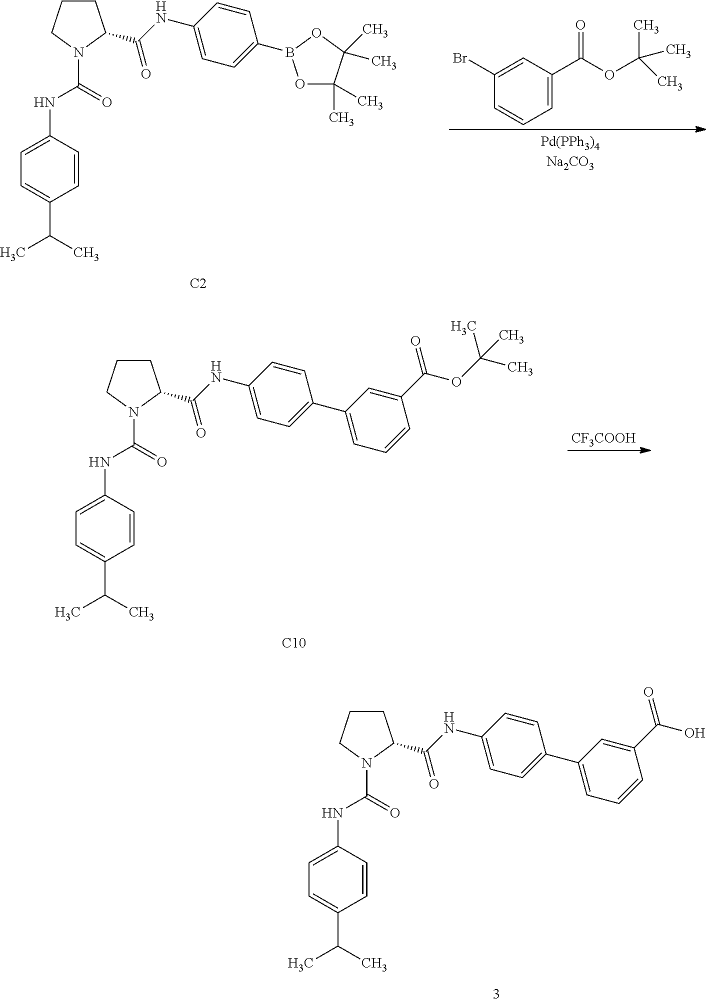

### Step 1. Synthesis of tert-butyl 4′-[(1-{[4-(propan-2-yl)phenyl]carbamoyl}-D-prolyl)amino][1,1′-biphenyl]-3-carboxylate (C10)

A vial containing C2 (250 mg, 0.524 mmol), sodium carbonate (98%, 170 mg, 1.57 mmol), tetrakis(triphenylphosphine)palladium(0) (45.4 mg, 39.3 μmol), and tert-butyl 3-bromobenzoate (202 mg, 0.786 mmol) was flushed three times with nitrogen, whereupon 1,2-dimethoxyethane (2.6 mL) and water (0.7 mL) were added. After the reaction mixture had been heated at 90° C. for 24 hours, LCMS analysis indicated conversion to C10: LCMS m/z 528.5 [M+H]⁺. Water was added, and the resulting mixture was extracted with ethyl acetate; the combined organic layers were washed with saturated aqueous sodium chloride solution, dried over sodium sulfate, filtered, and concentrated in vacuo. Silica gel chromatography (Gradient: 0% to 35% ethyl acetate in heptane) provided C10 as a white solid. Yield: 134 mg, 0.254 mmol, 48%. ¹H NMR (400 MHz, DMSO-d₆) δ 10.11 (s, 1H), 8.19 (s, 1H), 8.12-8.09 (m, 1H), 7.89 (br d, J=7.8 Hz, 1H), 7.85 (br d, J=7.8 Hz, 1H), 7.69 (AB quartet, J_{AB}=8.7 Hz, Δv_{AB}=32.9 Hz, 4H), 7.56 (t, J=7.8 Hz, 1H), 7.40 (d, J=8.6 Hz, 2H), 7.09 (d, J=8.6 Hz, 2H), 4.48 (dd, J=8.3, 3.6 Hz, 1H), 3.70-3.60 (m, 1H), 3.56-3.46 (m, 1H), 2.80 (septet, J=6.9 Hz, 1H), 2.25-2.13 (m, 1H), 2.11-1.88 (m, 3H), 1.57 (s, 9H), 1.16 (d, J=6.9 Hz, 6H).

### Step 2. Synthesis of 4′-[(1-{[4-(propan-2-yl)phenyl]carbamoyl}-D-prolyl)amino][1,1′-biphenyl]-3-carboxylic acid (3)

To a solution of C10 (134 mg, 0.254 mmol) in dichloromethane (1.3 mL) was added trifluoroacetic acid (0.194 mL, 2.52 mmol), and the reaction mixture was allowed to stir at room temperature. After 23 hours, LCMS analysis indicated complete conversion to 3: LCMS m/z 472.4 [M+H]⁺. The reaction mixture was diluted with dichloromethane and treated with saturated aqueous sodium bicarbonate solution. The aqueous layer was then acidified to pH 2 by addition of 1 M hydrochloric acid, and extracted with dichloromethane, whereupon the combined extracts were washed with saturated aqueous sodium chloride solution, dried over sodium sulfate, filtered, and concentrated in vacuo. The resulting solid was mixed with dichloromethane, stirred for 1 minute, and collected via filtration, affording 4′-[(1-{[4-(propan-2-yl)phenyl]carbamoyl}-D-prolyl)amino][1,1′-biphenyl]-3-carboxylic acid (3) as a white solid. Yield: 65 mg, 0.138 mmol, 54%. ¹H NMR (600 MHz, DMSO-d₆) δ 13.05 (br s, 1H), 10.11 (s, 1H), 8.19 (s, 1H), 8.16 (br s, 1H), 7.92-7.87 (m, 2H), 7.70 (AB quartet, J_{AB}=8.6 Hz, Δv_{AB}=42.1 Hz, 4H), 7.57 (t, J=7.7 Hz, 1H), 7.40 (d, J=8.6 Hz, 2H), 7.09 (d, J=8.5 Hz, 2H), 4.47 (dd, J=8.3, 3.7 Hz, 1H), 3.68-3.62 (m, 1H), 3.54-3.48 (m, 1H), 2.80 (septet, J=6.9 Hz, 1H), 2.23-2.15 (m, 1H), 2.08-1.99 (m, 1H), 1.99-1.90 (m, 2H), 1.16 (d, J=6.9 Hz, 6H).

### Example 4

### 4′-[(1-{[4-(Propan-2-yl)phenyl]carbamoyl}-D-prolyl)amino][1,1′-biphenyl]-4-carboxylic acid (4)

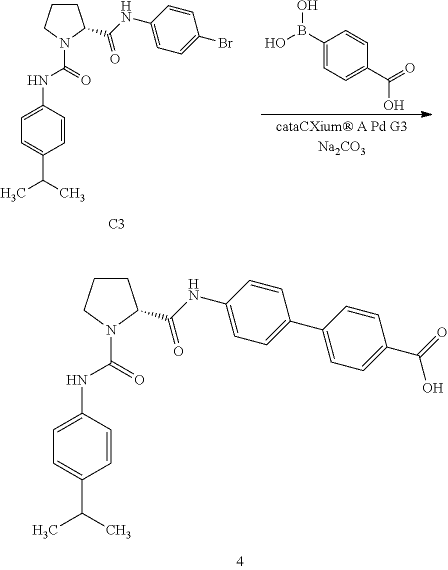

To a mixture of C3 (1.50 g, 3.49 mmol) and 4-boronobenzoic acid (694 mg, 4.18 mmol) in 1,4-dioxane (22 mL) was added aqueous sodium carbonate solution (2 M; 5.23 mL, 10.5 mmol), followed by mesylate[(di(1-adamantyl)-n-butylphosphine)-2-(2′-amino-1,1′-biphenyl)]palladium(II) (cataCXium® A Pd G3; 127 mg, 0.174 mmol). After the reaction mixture had been degassed for 5 minutes, it was heated at 90° C. overnight and then cooled to room temperature. The pH was adjusted to 4 to 5 via addition of 1 M hydrochloric acid, and the resulting mixture was partitioned between ethyl acetate (200 mL) and water (100 mL); this produced an insoluble solid, which was isolated via filtration and washed with ethyl acetate. This material was triturated with propan-2-ol, first at 70° C. for 5 hours, then at room temperature for 20 hours, whereupon the suspension was filtered and the filter cake was washed with dichloromethane, affording 4′-[(1-{[4-(propan-2-yl)phenyl]carbamoyl}-D-prolyl)amino][1,1′-biphenyl]-4-carboxylic acid (4) as a white solid. Yield: 435 mg, 0.922 mmol, 26%. LCMS m/z 472.3 [M+H]⁺. ¹H NMR (400 MHz, DMSO-d₆) δ 12.90 (br s, 1H), 10.13 (s, 1H), 8.19 (s, 1H), 7.88 (AB quartet, J_{AB}=8.4 Hz, Δv_{AB}=83.3 Hz, 4H), 7.72 (AB quartet, J_{AB}=9.0 Hz, Δv_{AB}=12.2 Hz, 4H), 7.40 (d, J=8.6 Hz, 2H), 7.09 (d, J=8.6 Hz, 2H), 4.47 (dd, J=8.3, 3.6 Hz, 1H), 3.70-3.60 (m, 1H), 3.56-3.47 (m, 1H), 2.80 (septet, J=6.9 Hz, 1H), 2.26-2.12 (m, 1H), 2.10-1.87 (m, 3H), 1.16 (d, J=6.9 Hz, 6H).

The combined organic layers from above were washed sequentially with water (2×50 mL) and saturated aqueous sodium chloride solution, dried over magnesium sulfate, filtered, and concentrated in vacuo. Purification via silica gel chromatography (Gradient: 0% to 5% methanol in dichloromethane), followed by trituration with a mixture of dichloromethane and methanol at room temperature for 20 hours, provided additional 4′-[(1-{[4-(propan-2-yl)phenyl]carbamoyl}-D-prolyl)amino][1,1′-biphenyl]-4-carboxylic acid (4) as a white solid. Yield: 410 mg, 0.869 mmol, 25%; combined yield: 51%.

### Alternate Synthesis of Example 4

### 4′-[(1-{[4-(Propan-2-yl)phenyl]carbamoyl}-D-prolyl)amino][1,1′-biphenyl]-4-carboxylic acid (4)

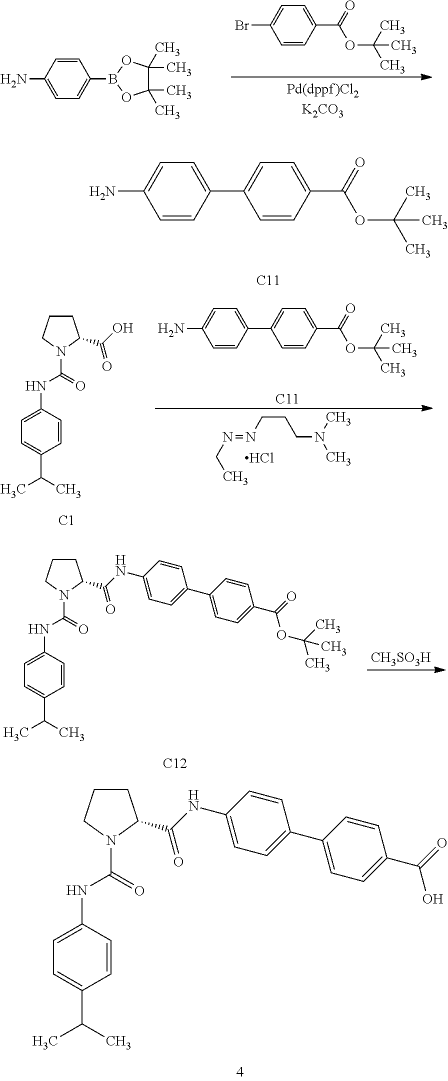

### Step 1. Synthesis of tert-butyl 4′-amino[1,1′-biphenyl]-4-carboxylate (C11)

To a solution of 4-(4,4,5,5-tetramethyl-1,3,2-dioxaborolan-2-yl)aniline (20.0 g, 91.3 mmol) in a mixture of 1,4-dioxane (400 mL) and water (100 mL) were added tert-butyl 4-bromobenzoate (25.8 g, 100 mmol) and potassium carbonate (37.8 g, 274 mmol). [1,1′-Bis(diphenylphosphino)ferrocene]dichloropalladium(II) (3.34 g, 4.56 mmol) was then added; the reaction mixture was heated at 95° C. for 16 hours, whereupon it was filtered. The filtrate was concentrated under reduced pressure, and the residue was diluted with water (500 mL) and extracted with ethyl acetate (2×500 mL). After the combined organic layers had been washed with saturated aqueous sodium chloride solution (2×500 mL), they were dried over sodium sulfate, filtered, and concentrated in vacuo. Silica gel chromatography (Gradient: 0% to 50% ethyl acetate in petroleum ether) provided a solid, which was treated with propan-2-yl acetate (20 mL) and heptane (80 mL) and stirred for 30 minutes before collection of the solid via filtration. The filter cake was washed with heptane (3×15 mL) to afford C11 as a pinkish-white solid. Yield: 18.5 g, 68.7 mmol, 75%. LCMS m/z 270.1 [M+H]⁺. ¹H NMR (400 MHz, DMSO-d₆) δ 7.88 (d, J=8.4 Hz, 2H), 7.65 (d, J=8.4 Hz, 2H), 7.45 (d, J=8.5 Hz, 2H), 6.66 (d, J=8.5 Hz, 2H), 5.39 (s, 2H), 1.55 (s, 9H).

### Step 2. Synthesis of tert-butyl 4′-[(1-{[4-(propan-2-yl)phenyl]carbamoyl}-D-prolyl)amino][1,1′-biphenyl]-4-carboxylate (C12)

1-[3-(Dimethylamino)propyl]-3-ethylcarbodiimide hydrochloride (3.46 g, 18.0 mmol) was added to a solution of C1 (3.95 g, 14.3 mmol) and C11 (4.05 g, 15.0 mmol) in N,N-dimethylacetamide (38 mL). After 1.5 hours, LCMS analysis indicated the presence of product: LCMS m/z 528.5 [M+H]⁺. Water (50 mL) was added, and the mixture was stirred for 8 minutes before being filtered; the collected solids were washed with water and stirred in diethyl ether (60 mL) for 10 minutes. Solids were isolated again by filtration, suspended in a solution of methanol in dichloromethane (5%, 50 mL), stirred for 25 minutes, and filtered. Washing of the filter cake with dichloromethane afforded C12 as a white solid. Yield: 5.43 g, 10.3 mmol, 72%. ¹H NMR (400 MHz, DMSO-d₆) δ 10.14 (s, 1H), 8.19 (s, 1H), 7.94 (d, J=8.5 Hz, 2H), 7.78 (d, J=8.5 Hz, 2H), 7.72 (AB quartet, J_{AB}=8.9 Hz, Δv_{AB}=15.6 Hz, 4H), 7.40 (d, J=8.6 Hz, 2H), 7.09 (d, J=8.6 Hz, 2H), 4.47 (dd, J=8.2, 3.6 Hz, 1H), 3.69-3.60 (m, 1H), 3.56-3.47 (m, 1H), 2.79 (septet, J=6.9 Hz, 1H), 2.25-2.13 (m, 1H), 2.10-1.88 (m, 3H), 1.56 (s, 9H), 1.16 (d, J=6.9 Hz, 6H).

### Step 3. Synthesis of 4′-[(1-{[4-(propan-2-yl)phenyl]carbamoyl}-D-prolyl)amino][1,1′-biphenyl]-4-carboxylic acid (4)

Methanesulfonic acid (0.129 mL, 1.99 mmol) was added to a solution of C12 (1.00 g, 1.90 mmol) in 1,1,1,3,3,3-hexafluoropropan-2-ol (10 mL), and the reaction mixture was stirred at room temperature for 15 minutes, whereupon it was concentrated in vacuo. The residue was slurried in diethyl ether for 5 minutes; solids were collected via filtration, washed with diethyl ether, and suspended in propan-2-yl acetate (15 mL). Methanol (2 mL) was added, and the mixture was stirred at room temperature for 3 days. Isolation via filtration, followed by washing of the filter cake with propan-2-yl acetate (3 mL), provided 4′-[(1-{[4-(propan-2-yl)phenyl]carbamoyl}-D-prolyl)amino][1,1′-biphenyl]-4-carboxylic acid (4) as a solid. Yield: 668 mg, 1.42 mmol, 75%. LCMS m/z 472.4 [M+H]⁺. ¹H NMR (400 MHz, DMSO-d₆) 12.90 (v br s, 1H), 10.13 (s, 1H), 8.19 (s, 1H), 7.99 (d, J=8.5 Hz, 2H), 7.78 (d, J=8.5 Hz, 2H), 7.72 (AB quartet, J_{AB}=9.0 Hz, Δv_{AB}=12.7 Hz, 4H), 7.40 (d, J=8.6 Hz, 2H), 7.09 (d, J=8.6 Hz, 2H), 4.47 (dd, J=8.3, 3.6 Hz, 1H), 3.70-3.61 (m, 1H), 3.56-3.47 (m, 1H), 2.80 (septet, J=6.9 Hz, 1H), 2.25-2.13 (m, 1H), 2.10-1.88 (m, 3H), 1.16 (d, J=6.9 Hz, 6H).

### Example 5

### Ammonium 4-{6-[(1-{[4-(propan-2-yl)phenyl]carbamoyl}-D-prolyl)amino]pyridin-3-yl}benzoate (5)

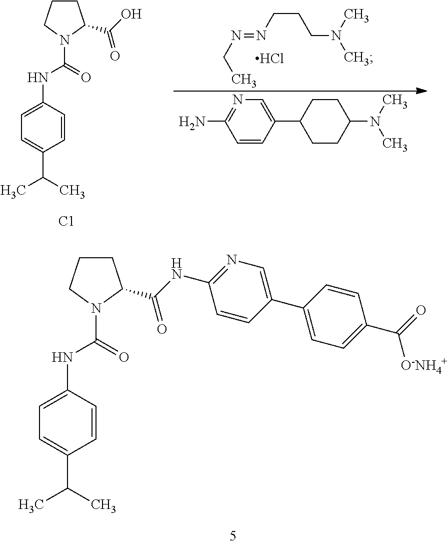

A mixture of C1 (50 mg, 0.18 mmol) and 1-[3-(dimethylamino)propyl]-3-ethylcarbodiimide hydrochloride (45.1 mg, 0.235 mmol) in dichloromethane (0.90 mL) was stirred for approximately 30 minutes, whereupon 4-(6-aminopyridin-3-yl)benzoic acid (38.8 mg, 0.181 mmol) was added, and the reaction mixture was stirred at room temperature overnight. It was then filtered; the filtrate was acidified by addition of 1 M hydrochloric acid and then extracted 4 times with ethyl acetate. The combined organic layers were washed with saturated aqueous sodium chloride solution, dried over sodium sulfate, filtered, and concentrated in vacuo. Purification using reversed-phase HPLC (Column: Waters XBridge C18, 19×100 mm, 5 μm; Mobile phase A: water containing 0.03% ammonium hydroxide; Mobile phase B: acetonitrile containing 0.03% ammonium hydroxide; Gradient: 5% to 95% B; Flow rate: 25 mL/minute) afforded ammonium 4-{6-[(1-{[4-(propan-2-yl)phenyl]carbamoyl}-D-prolyl)amino]pyridin-3-yl}benzoate (5). Yield: 1.0 mg, 2.0 μmol, 1%. LCMS m/z 473.4 [M+H]⁺. ¹H NMR (600 MHz, DMSO-d₆), characteristic peaks: δ 10.65 (s, 1H), 8.74-8.70 (m, 1H), 8.22 (s, 1H), 8.17 (br s, 2H), 8.02 (d, J=8.3 Hz, 2H), 7.85 (d, J=8.1 Hz, 2H), 7.38 (d, J=8.1 Hz, 2H), 7.09 (d, J=8.6 Hz, 2H), 4.64-4.59 (m, 1H), 3.66-3.60 (m, 1H, assumed; partially obscured by water peak), 2.80 (septet, J=7.0 Hz, 1H), 2.21-2.13 (m, 1H), 2.05-1.91 (m, 3H), 1.16 (d, J=6.9 Hz, 6H).

### Alternate Synthesis of Example 5 (free acid)

### 4-{6-[(1-{[4-(Propan-2-yl)phenyl]carbamoyl}-D-prolyl)amino]pyridin-3-yl}benzoic acid (5, free acid)

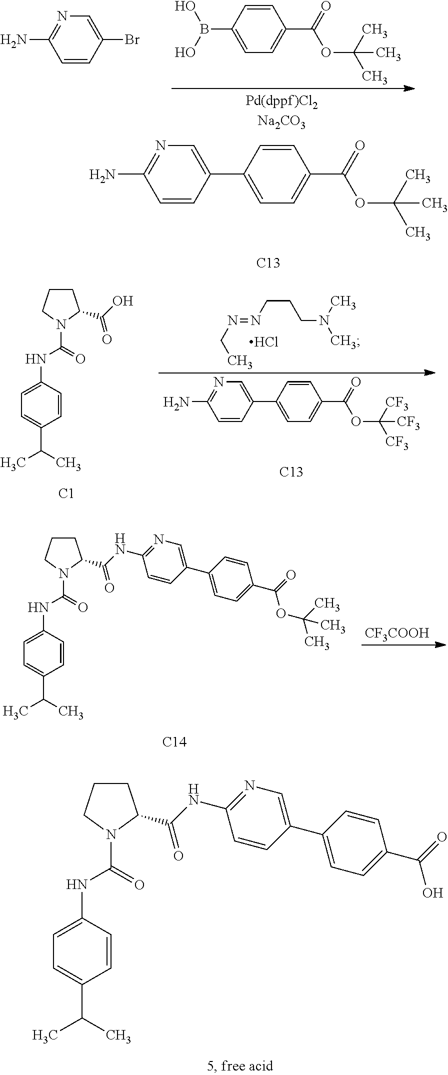

### Step 1. Synthesis of tert-butyl 4-(6-aminopyridin-3-yl)benzoate (C13)

Sodium carbonate (3.68 g, 34.7 mmol) and water (9 mL) were added to a solution of 5-bromopyridin-2-amine (2.00 g, 11.6 mmol) and [4-(tert-butoxycarbonyl)phenyl]boronic acid (2.57 g, 11.6 mmol) in 1,4-dioxane (36 mL). The solution was degassed for 10 minutes with nitrogen, whereupon [1,1′-bis(diphenylphosphino)ferrocene]dichloropalladium(II) (423 mg, 0.578 mmol) was added and the reaction mixture was heated at 80° C. for 18 hours. After it had cooled to room temperature, the reaction mixture was partitioned between water (60 mL) and ethyl acetate (30 mL). The aqueous layer was extracted with ethyl acetate (2×30 mL), and the combined organic layers were dried over sodium sulfate, filtered, and concentration in vacuo; silica gel chromatography (Gradient: 40% to 100% ethyl acetate in heptane) afforded C13 as a yellow solid. Yield: 2.37 g, 8.77 mmol, 76%. ¹H NMR (400 MHz, chloroform-d) δ 8.34 (d, J=2.2 Hz, 1H), 8.03 (d, J=8.5 Hz, 2H), 7.72 (dd, J=8.6, 2.4 Hz, 1H), 7.54 (d, J=8.5 Hz, 2H), 6.62 (d, J=8.6 Hz, 1H), 4.71 (br s, 2H), 1.61 (s, 9H).

### Step 2. Synthesis of tert-butyl 4-{6-[(1-{[4-(propan-2-yl)phenyl]carbamoyl}-D-prolyl)amino]pyridin-3-yl}benzoate (C14)

1-[3-(Dimethylamino)propyl]-3-ethylcarbodiimide hydrochloride (1.11 g, 5.79 mmol) was added to a 0° C. solution of C1 (1.33 g, 4.81 mmol) in dichloromethane (16 mL). The reaction mixture was stirred at 0° C. for 40 minutes, whereupon a solution of C13 (1.30 g, 4.81 mmol) in dichloromethane (5 mL) was added. Stirring was continued at 0° C. for 70 minutes, then at room temperature for 2.25 hours; solids were collected via filtration to provide C14 as a white solid. Yield: 797 mg, 1.51 mmol, 31%. LCMS m/z 529.5 [M+H]⁺. ¹H NMR (600 MHz, DMSO-d₆) δ 10.67 (s, 1H), 8.74-8.71 (m, 1H), 8.22 (s, 1H), 8.18 (br s, 2H), 7.97 (d, J=8.3 Hz, 2H), 7.85 (d, J=8.3 Hz, 2H), 7.39 (d, J=8.5 Hz, 2H), 7.09 (d, J=8.4 Hz, 2H), 4.66-4.60 (m, 1H), 3.67-3.60 (m, 1H), 3.55-3.48 (m, 1H), 2.85-2.76 (m, 1H), 2.22-2.13 (m, 1H), 2.06-1.91 (m, 3H), 1.57 (s, 9H), 1.16 (d, J=6.9 Hz, 6H).

### Step 3. Synthesis of 4-{6-[(1-{[4-(propan-2-yl)phenyl]carbamoyl}-D-prolyl)amino]pyridin-3-yl}benzoic acid (5, free acid)

Trifluoroacetic acid (98.1 mg, 0.860 mmol) was added drop-wise to a solution of C14 (455 mg, 0.861 mmol) in dichloromethane (4.3 mL). After the reaction mixture had been stirred at room temperature for 3.5 hours, it was concentrated in vacuo, then co-evaporated three times with toluene. The resulting solid was treated with propan-2-yl acetate (7.5 mL), followed by methanol (1 mL), and the slurry was stirred overnight at room temperature. Filtration afforded 4-{6-[(1-{[4-(propan-2-yl)phenyl]carbamoyl}-D-prolyl)amino]pyridin-3-yl}benzoic acid (5, free acid) as a white solid. Yield: 374 mg, 0.791 mmol, 92%. LCMS m/z 473.4 [M+H]⁺. ¹H NMR (600 MHz, DMSO-d₆), characteristic peaks: δ 10.67 (s, 1H), 8.74-8.72 (m, 1H), 8.22 (s, 1H), 8.19-8.17 (m, 2H), 8.02 (d, J=8.4 Hz, 2H), 7.85 (d, J=8.3 Hz, 2H), 7.39 (d, J=8.4 Hz, 2H), 7.09 (d, J=8.5 Hz, 2H), 4.66-4.58 (m, 1H), 3.67-3.61 (m, 1H), 3.54-3.48 (m, 1H, assumed; partially obscured by water peak), 2.85-2.76 (m, 1H), 2.21-2.13 (m, 1H), 1.16 (d, J=6.8 Hz, 6H).

### Example 6

### 3′-Fluoro-4′-[(1-{[4-(propan-2-yl)phenyl]carbamoyl}-D-prolyl)amino][1,1′-biphenyl]-4-carboxylic acid (6)

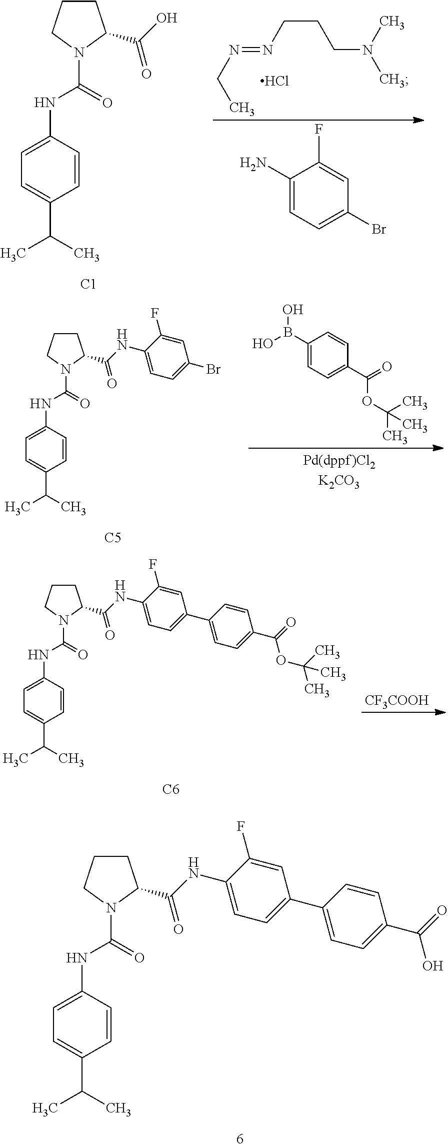

### Step 1. Synthesis of (2R)—N²-(4-bromo-2-fluorophenyl)-N¹-[4-(propan-2-yl)phenyl]pyrrolidine-1,2-dicarboxamide (C5)

1-[3-(Dimethylamino)propyl]-3-ethylcarbodiimide hydrochloride (416 mg, 2.17 mmol) was added to a 0° C. mixture of C1 (500 mg, 1.81 mmol) in dichloromethane (7.9 mL). The reaction mixture was stirred at 0° C. for 1 hour, whereupon 4-bromo-2-fluoroaniline (344 mg, 1.81 mmol) was added in a single portion, and stirring was continued at 0° C. for 80 minutes. The cooling bath was then removed; after the reaction mixture had been stirred overnight, it was poured into water and extracted three times with dichloromethane. The combined organic layers were washed sequentially with water and saturated aqueous sodium sulfate solution, dried over magnesium sulfate, filtered, and concentrated in vacuo to provide C5 as a white foam. Yield: 830 mg, quantitative. LCMS m/z 448.3 (bromine isotope pattern observed) [M+H]⁺. ¹H NMR (600 MHz, DMSO-d₆) δ 9.94 (s, 1H), 8.21 (s, 1H), 7.90 (t, J=8.6 Hz, 1H), 7.59 (dd, J=10.4, 2.2 Hz, 1H), 7.39 (d, J=8.4 Hz, 2H), 7.36 (br d, J=8.9 Hz, 1H), 7.09 (d, J=8.4 Hz, 2H), 4.60 (dd, J=8.2, 2.8 Hz, 1H), 3.64-3.59 (m, 1H), 3.51-3.45 (m, 1H), 2.81 (septet, J=7.0 Hz, 1H), 2.17-2.08 (m, 1H), 2.02-1.91 (m, 3H), 1.17 (d, J=6.9 Hz, 6H).

### Step 2. Synthesis of tert-butyl 3′-fluoro-4′-[(1-{[4-(propan-2-yl)phenyl]carbamoyl}-D-prolyl)amino][1,1′-biphenyl]-4-carboxylate (C6)

A mixture of C5 (400 mg, 0.892 mmol), [4-(tert-butoxycarbonyl)phenyl]boronic acid (218 mg, 0.982 mmol), potassium carbonate (370 mg, 2.68 mmol) and [1,1′-bis(diphenylphosphino)ferrocene]dichloropalladium(II) [Pd(dppf)Cl₂; 32.6 mg, 44.6 μmol] in a mixture of 1,4-dioxane (3.0 mL) and water (0.5 mL) was sparged three times with nitrogen, and then heated at 65° C. for 3 hours. After the reaction mixture had cooled to room temperature, it was filtered through a plug of diatomaceous earth. The filtrate was partitioned between ethyl acetate and water, and the aqueous layer was extracted twice with ethyl acetate. The combined organic layers were washed with saturated aqueous sodium sulfate solution, dried over magnesium sulfate, filtered, and concentrated in vacuo; silica gel chromatography (Gradient: 0% to 80% ethyl acetate in heptane) afforded C6 as a white solid. Yield: 101 mg, 0.185 mmol, 21%. LCMS m/z 546.5 [M+H]⁺. ¹H NMR (600 MHz, DMSO-d₆), characteristic peaks: δ 9.99 (s, 1H), 8.22 (s, 1H), 8.10 (t, J=8.4 Hz, 1H), 7.89 (AB quartet, J_{AB}=8.4 Hz, Δv_{AB}=72.3 Hz, 4H), 7.70 (br dd, J=12.2, 2 Hz, 1H), 7.58 (br d, J=8.4 Hz, 1H), 7.40 (d, J=8.3 Hz, 2H), 7.10 (d, J=8.4 Hz, 2H), 4.67-4.63 (m, 1H), 3.67-3.60 (m, 1H), 3.53-3.47 (m, 1H), 2.84-2.78 (m, 1H), 2.19-2.10 (m, 1H), 1.56 (s, 9H), 1.17 (d, J=6.8 Hz, 6H).

### Step 3. Synthesis of 3′-fluoro-4′-[(1-{[4-(propan-2-yl)phenyl]carbamoyl}-D-prolyl)amino][1,1′-biphenyl]-4-carboxylic acid (6)

Trifluoroacetic acid (0.2 mL) was added drop-wise to a solution of C6 (88.0 mg, 0.161 mmol) in dichloromethane (0.8 mL). The reaction mixture was stirred at room temperature for 2.5 hours, whereupon it was concentrated in vacuo and then co-evaporated three times with toluene. The resulting material was combined with the product of a similar reaction carried out using C6 (43 mg, 79 μmol) and purified using silica gel chromatography (Gradient: 0% to 10% methanol in dichloromethane), providing 3′-fluoro-4′-[(1-{[4-(propan-2-yl)phenyl]carbamoyl}-D-prolyl)amino][1,1′-biphenyl]-4-carboxylic acid (6) as a white solid. Combined yield: 60 mg, 0.12 mmol, 75%. LCMS m/z 490.5 [M+H]⁺. ¹H NMR (600 MHz, DMSO-d₆) δ 12.98 br (s, 1H), 9.99 (s, 1H), 8.22 (s, 1H), 8.10 (t, J=8.4 Hz, 1H), 8.00 (d, J=8.5 Hz, 2H), 7.83 (d, J=8.5 Hz, 2H), 7.70 (dd, J=12.3, 2.1 Hz, 1H), 7.58 (dd, J=8.5, 2.1 Hz, 1H), 7.41 (d, J=8.6 Hz, 2H), 7.10 (d, J=8.6 Hz, 2H), 4.65 (dd, J=8.2, 2.8 Hz, 1H), 3.67-3.60 (m, 1H), 3.53-3.47 (m, 1H), 2.81 (septet, J=6.9 Hz, 1H), 2.20-2.10 (m, 1H), 2.05-1.93 (m, 3H), 1.17 (d, J=6.9 Hz, 6H).

### Example 7

### Ammonium 4′-({1-[(4-cyclopropylphenyl)carbamoyl]-D-prolyl}amino)[1,1′-biphenyl]-4-carboxylate (7)

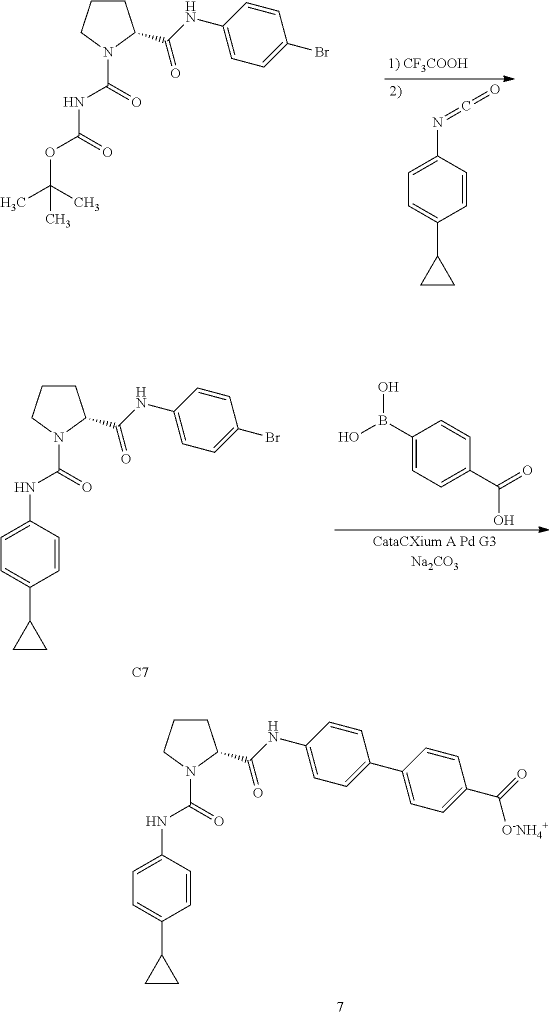

### Step 1. Synthesis of (2R)—N²-(4-bromophenyl)-N¹-(4-cyclopropylphenyl)pyrrolidine-1,2-dicarboxamide (C7)

Trifluoroacetic acid (0.414 mL, 5.37 mmol) was added to a 0° C. solution of tert-butyl (2R)-2-[(4-bromophenyl)carbamoyl]pyrrolidine-1-carboxylate (see V. H. Thorat et al., Eur. J. Org. Chem. 2013, 3529-3542; 100 mg, 0.271 mmol) in dichloromethane (2.7 mL). After the reaction mixture had been stirred at 0° C. for 30 minutes, then at room temperature for 1.5 hours, it was concentrated under a stream of nitrogen, then under high vacuum. The residue was dissolved in tetrahydrofuran (1.5 mL), treated with 1-cyclopropyl-4-isocyanatobenzene (64.7 mg, 0.406 mmol), and allowed to stir at room temperature for approximately 2 hours. After removal of solvent under a stream of nitrogen, the residue was purified via silica gel chromatography (Gradient: 0% to 50% ethyl acetate in dichloromethane) to provide C7 as a solid. Yield: 67 mg, 0.16 mmol, 59%. LCMS m/z 428.2 (bromine isotope pattern observed) [M+H]⁺. ¹H NMR (400 MHz, DMSO-d₆) δ 10.10 (s, 1H), 8.17 (s, 1H), 7.52 (AB quartet, J_{AB}=8.8 Hz, Δv_{AB}=43.6 Hz, 4H), 7.36 (d, J=8.6 Hz, 2H), 6.92 (d, J=8.6 Hz, 2H), 4.42 (dd, J=8.2, 3.7 Hz, 1H), 3.66-3.57 (m, 1H), 3.54-3.44 (m, 1H), 2.22-2.10 (m, 1H), 2.07-1.86 (m, 3H), 1.86-1.77 (m, 1H), 0.90-0.83 (m, 2H), 0.61-0.54 (m, 2H).

### Step 2. Synthesis of ammonium 4′-({1-[(4-cyclopropylphenyl)carbamoyl]-D-prolyl}amino)[1,1′-biphenyl]-4-carboxylate (7)

A mixture of C7 (67 mg, 0.16 mmol), 4-boronobenzoic acid (31.1 mg, 0.187 mmol), and mesylate[(di(1-adamantyl)-n-butylphosphine)-2-(2′-amino-1,1′-biphenyl)]palladium(II) (cataCXium® A Pd G3; 11.4 mg, 15.6 μmol) in 1,4-dioxane (1.6 mL) was sparged with nitrogen for 10 minutes. A degassed aqueous solution of sodium carbonate (2.0 M; 0.274 mL, 0.548 mmol) was added, and the reaction mixture was heated at 90° C. overnight. After the reaction mixture had been cooled and acidified by addition of 1 M hydrochloric acid, it was diluted with ethyl acetate. The aqueous layer was extracted with ethyl acetate, and the combined organic layers were dried over sodium sulfate, filtered, and concentrated in vacuo; purification via reversed-phase HPLC (Column: Waters XBridge C18, 19×100 mm, 5 μm; Mobile phase A: water containing 0.03% ammonium hydroxide; Mobile phase B: acetonitrile containing 0.03% ammonium hydroxide; Gradient: 5% to 95% B; Flow rate: 25 mL/minute) afforded ammonium 4′-({1-[(4-cyclopropylphenyl)carbamoyl]-D-prolyl}amino)[1,1′-biphenyl]-4-carboxylate (7). Yield: 8.1 mg, 17 μmol, 11%. LCMS m/z 470.5 [M+H]⁺. ¹H NMR (600 MHz, DMSO-d₆) δ 10.13 (s, 1H), 8.18 (s, 1H), 7.99 (d, J=8.5 Hz, 2H), 7.78 (d, J=8.5 Hz, 2H), 7.72 (AB quartet, J_{AB}=8.9 Hz, Δv_{AB}=16.1 Hz, 4H), 7.37 (d, J=8.6 Hz, 2H), 6.93 (d, J=8.7 Hz, 2H), 4.46 (dd, J=8.3, 3.7 Hz, 1H), 3.64 (ddd, J=9.3, 7.6, 4.7 Hz, 1H), 3.53-3.47 (m, 1H), 2.23-2.14 (m, 1H), 2.07-1.98 (m, 1H), 1.98-1.89 (m, 2H), 1.82 (tt, J=8.4, 5.1 Hz, 1H), 0.89-0.84 (m, 2H), 0.59-0.55 (m, 2H).

### Example 16

### Ammonium 4′-[(1-{[4-(trifluoromethyl)phenyl]carbamoyl}-D-prolyl)amino][1,1′-biphenyl]-4-carboxylate (16)

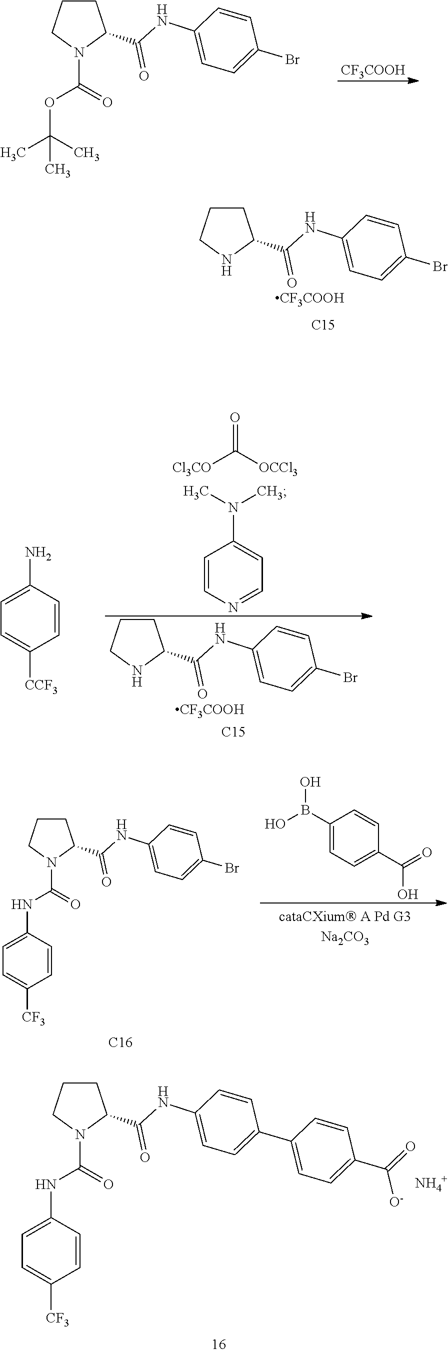

### Step 1. Synthesis of N-(4-bromophenyl)-D-prolinamide, trifluoroacetate salt (C15)

Trifluoroacetic acid (2.07 mL, 26.9 mmol) was added to a 0° C. solution of tert-butyl (2R)-2-[(4-bromophenyl)carbamoyl]pyrrolidine-1-carboxylate (see V. H. Thorat et al., Eur. J. Org. Chem. 2013, 3529-3542; 500 mg, 1.35 mmol) in dichloromethane (13.5 mL). After the reaction mixture had been stirred at 0° C. for 30 minutes, and then at room temperature for 1.5 hours, it was concentrated under a stream of nitrogen, providing C15 as a solid. Yield: 364 mg, 0.950 mmol, 70%. LCMS m/z 269.5 (bromine isotope pattern observed) [M+H]⁺.

### Step 2. Synthesis of (2R)—N²-(4-bromophenyl)-N¹-[4-(trifluoromethyl)phenyl]pyrrolidine-1,2-dicarboxamide (C16)

To a solution of 4-(trifluoromethyl)aniline (40.2 mg, 0.249 mmol) in dichloromethane (1 mL) was added a solution of bis(trichloromethyl) carbonate (25.6 mg, 86.3 μmol) in dichloromethane (0.5 mL), followed by 4-(dimethylamino)pyridine (93.8 mg, 0.768 mmol). After the reaction mixture had been stirred at room temperature for 1 hour, it was treated with a solution of C15 (73.5 mg, 0.192 mmol) in dichloromethane (1 mL). LCMS analysis, after an additional 40 minutes of stirring at room temperature, indicated conversion to C16: LCMS m/z 456.1 (bromine isotope pattern observed) [M+H]⁺. After an additional 30 minutes of stirring at room temperature, the reaction mixture was diluted with dichloromethane, washed sequentially with 1M hydrochloric acid and saturated aqueous sodium bicarbonate solution, dried over sodium sulfate, filtered, and concentrated in vacuo. Silica gel chromatography (Gradient: 0% to 50% ethyl acetate in dichloromethane) afforded C16 as a solid. Yield: 43 mg, 94 μmol, 49%. ¹H NMR (400 MHz, DMSO-d₆) δ 10.14 (s, 1H), 8.68 (s, 1H), 7.74 (d, half of AB quartet, J=8.5 Hz, 2H), 7.62-7.54 (m, 4H), 7.48 (d, half of AB quartet, J=8.9 Hz, 2H), 4.45 (dd, J=8.3, 3.8 Hz, 1H), 3.71-3.62 (m, 1H), 3.60-3.51 (m, 1H), 2.26-2.14 (m, 1H), 2.08-1.86 (m, 3H).

### Step 3. Synthesis of ammonium 4′-[(1-{[4-(trifluoromethyl)phenyl]carbamoyl}-D-prolyl)amino][1,1′-biphenyl]-4-carboxylate (16)

A mixture of C16 (43.0 mg, 94.2 μmol), 4-boronobenzoic acid (18.8 mg, 0.113 mmol), and mesylate[(di(1-adamantyl)-n-butylphosphine)-2-(2′-amino-1,1′-biphenyl)]palladium(II) (cataCXium® A Pd G3; 6.86 mg, 9.42 μmol) in 1,4-dioxane (0.9 mL) was sparged with nitrogen for 10 minutes, whereupon a degassed solution of aqueous sodium carbonate (2.0 M; 0.165 mL, 0.330 mmol) was added. After the reaction mixture had been heated at 90° C. overnight, it was acidified by addition of 1 M hydrochloric acid, and diluted with ethyl acetate. The aqueous layer was extracted with ethyl acetate, and the combined organic layers were dried over sodium sulfate, filtered, and concentrated in vacuo. The resulting solid was purified using reversed-phase HPLC (Column: Waters XBridge C18, 19×100 mm, 5 μm; Mobile phase A: 0.03% ammonium hydroxide in water (v/v); Mobile phase B: 0.03% ammonium hydroxide in acetonitrile (v/v); Gradient: 5.0% to 95% B; Flow rate: 25 mL/min) to provide ammonium 4′-[(1-{[4-(trifluoromethyl)phenyl]carbamoyl}-D-prolyl)amino][1,1′-biphenyl]-4-carboxylate (16). Yield: 17.9 mg, 36.0 μmol, 38%. LCMS m/z 498.4 [M+H]⁺. ¹H NMR (600 MHz, DMSO-d₆) δ 12.9 (v br s, 1H), 10.17 (s, 1H), 8.70 (s, 1H), 7.99 (d, J=8.5 Hz, 2H), 7.78 (d, J=8.5 Hz, 2H), 7.77-7.73 (m, 4H), 7.71 (d, half of AB quartet, J=8.9 Hz, 2H), 7.58 (d, J=8.7 Hz, 2H), 4.50 (dd, J=8.4, 3.9 Hz, 1H), 3.68 (ddd, J=9.4, 7.6, 4.9 Hz, 1H), 3.60-3.54 (m, 1H), 2.27-2.18 (m, 1H), 2.08-2.00 (m, 1H), 2.00-1.90 (m, 2H).

### Alternate Synthesis of Example 16 (free acid)

### 4′-[(1-{[4-(Trifluoromethyl)phenyl]carbamoyl}-D-prolyl)amino][1,1′-biphenyl]-4-carboxylic acid (16, free acid)

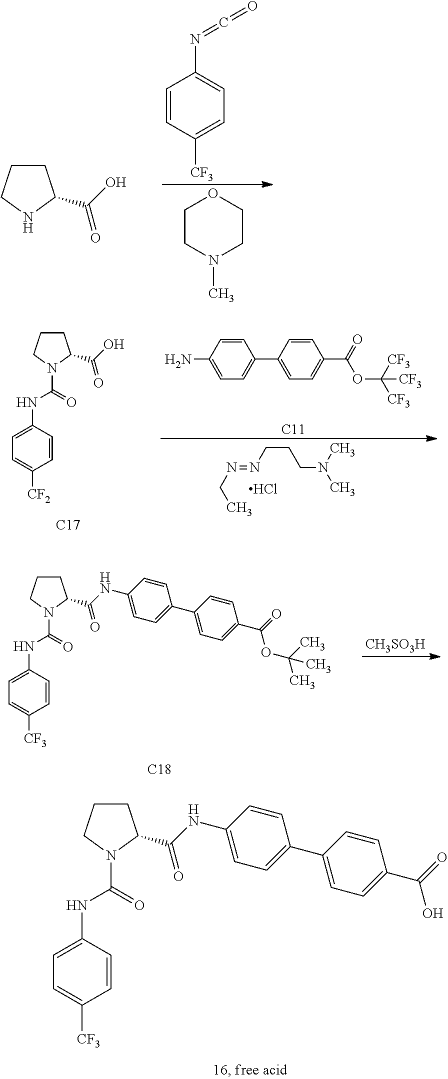

### Step 1. Synthesis of 1-{[4-(trifluoromethyl)phenyl]carbamoyl}-D-proline (C17)

4-Methylmorpholine (0.846 mL, 778 mg, 7.69 mmol) and 1-isocyanato-4-(trifluoromethyl)benzene (1.20 g, 6.41 mmol) were added to a solution of D-proline (886 mg, 7.70 mmol) in tetrahydrofuran (21.4 mL). The reaction mixture was stirred at room temperature for 3 hours, whereupon it was diluted with water (20 mL) and adjusted to a pH of 7 to 8 by addition of solid sodium bicarbonate. After the aqueous layer had been washed with methyl tert-butyl ether (2×20 mL), it was acidified to pH 2 to 3 with concentrated hydrochloric acid and extracted with ethyl acetate (3×20 mL). The combined ethyl acetate layers were washed with saturated aqueous sodium chloride solution (60 mL), dried over magnesium sulfate, and concentrated in vacuo, affording C17 as a white solid. Yield: 1.00 g, 3.31 mmol, 52%. LCMS m/z 301.1 [M−H]⁻. ¹H NMR (400 MHz, DMSO-d₆) δ 8.68 (s, 1H), 7.73 (d, J=8.5 Hz, 2H), 7.58 (d, J=8.6 Hz, 2H), 4.40-4.28 (m, 1H), 3.63-3.54 (m, 1H), 3.54-3.45 (m, 1H), 2.24-2.12 (m, 1H), 1.96-1.85 (m, 3H).

### Step 2. Synthesis of tert-butyl 4′-[(1-{[4-(trifluoromethyl)phenyl]carbamoyl}-D-prolyl)amino][1,1′-biphenyl]-4-carboxylate (C18)

A solution of C17 (400 mg, 1.32 mmol) and C11 (374 mg, 1.39 mmol) in N,N-dimethylacetamide (3.3 mL) was treated with 1-[3-(dimethylamino)propyl]-3-ethylcarbodiimide hydrochloride (304 mg, 1.59 mmol). After 3 hours at room temperature, the reaction mixture was diluted with water (5 mL). The aqueous layer was extracted with ethyl acetate (3×5 mL), and the combined organic layers were washed sequentially with water (2×15 mL) and saturated aqueous sodium chloride solution (15 mL), dried over magnesium sulfate, filtered, and concentrated in vacuo. Trituration of the resulting material with dichloromethane (5 mL) provided C18. Yield: 330 mg, 0.596 mmol, 45%. LCMS m/z 554.4 [M+H]⁺. ¹H NMR (400 MHz, DMSO-d₆) δ 10.17 (s, 1H), 8.70 (s, 1H), 7.95 (d, J=8.5 Hz, 2H), 7.81-7.73 (m, 6H), 7.70 (d, half of AB quartet, J=8.9 Hz, 2H), 7.58 (d, J=8.6 Hz, 2H), 4.51 (dd, J=8.4, 3.8 Hz, 1H), 3.74-3.64 (m, 1H), 3.63-3.52 (m, 1H), 2.29-2.16 (m, 1H), 2.11-1.89 (m, 3H), 1.56 (s, 9H).

### Step 3. Synthesis of 4′-[(1-{[4-(trifluoromethyl)phenyl]carbamoyl}-D-prolyl)amino][1,1′-biphenyl]-4-carboxylic acid (16, free acid)

Methanesulfonic acid (45.0 μL, 0.693 mmol) was added to a solution of C18 (320 mg, 0.578 mmol) in 1,1,1,3,3,3-hexafluoropropan-2-ol (5.8 mL). After the reaction mixture had been stirred for 15 minutes at room temperature, it was treated with additional methanesulfonic acid (20 μL, 0.31 mmol), and stirring was continued for 10 minutes. The reaction mixture was cooled to 0° C. and treated with methanol (5 mL; pre-cooled to 0° C.); over 30 minutes, a precipitate formed. Filtration afforded a solid, which was slurried in ethanol (3 mL) at 50° C. for 8 hours, then allowed to cool to room temperature and sit for 72 hours. Solids were collected via filtration to afford 4′-[(1-{[4-(trifluoromethyl)phenyl]carbamoyl}-D-prolyl)amino][1,1′-biphenyl]-4-carboxylic acid (16, free acid) as a white solid. Yield: 192 mg, 0.386 mmol, 67%. LCMS m/z 498.2 [M+H]⁺. ¹H NMR (400 MHz, DMSO-d₆) δ 10.17 (s, 1H), 8.70 (s, 1H), 7.99 (d, J=8.5 Hz, 2H), 7.80-7.73 (m, 6H), 7.71 (d, half of AB quartet, J=8.9 Hz, 2H), 7.58 (d, J=8.7 Hz, 2H), 4.50 (dd, J=8.3, 3.7 Hz, 1H), 3.74-3.64 (m, 1H), 3.62-3.52 (m, 1H), 2.28-2.16 (m, 1H), 2.11-1.89 (m, 3H).

### Example 17

### 5-{4-[(1-{[3-Methyl-4-(trifluoromethyl)phenyl]carbamoyl}-D-prolyl)amino]phenyl}pyridine-2-carboxylic acid (17)

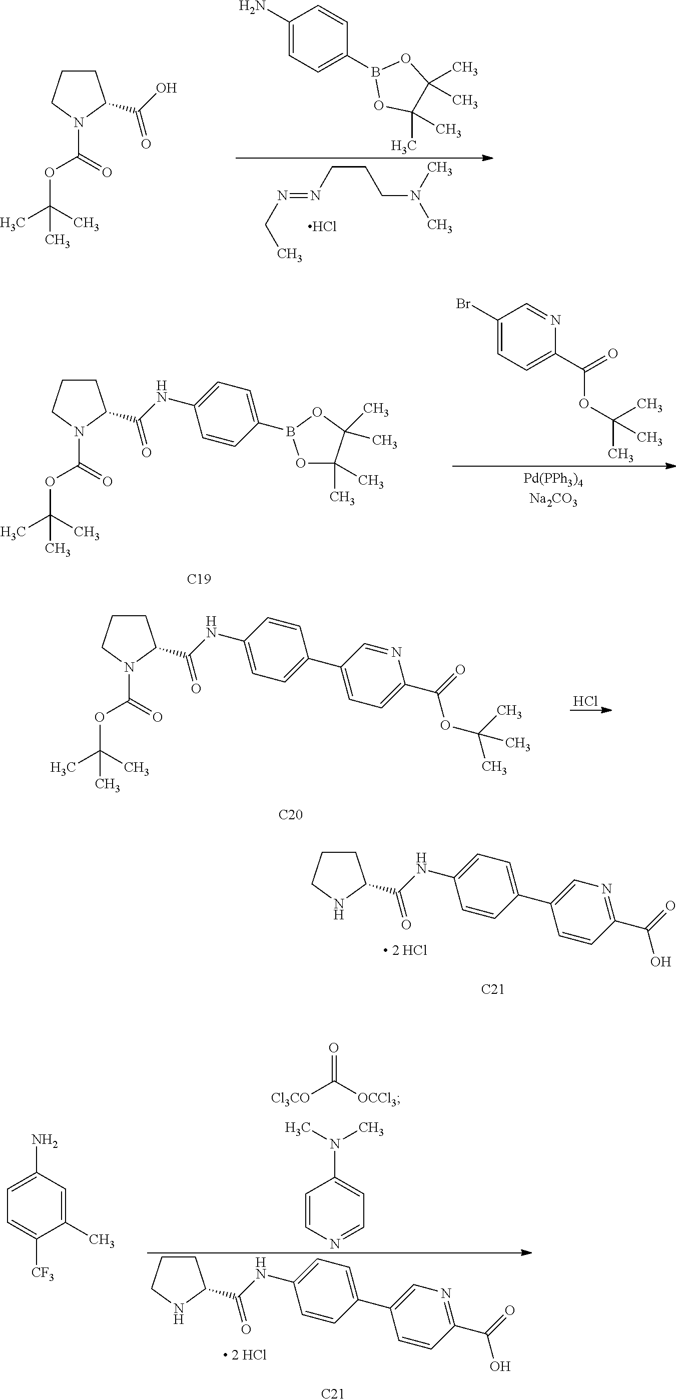

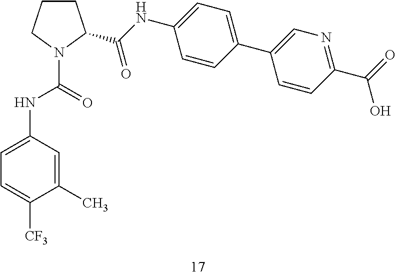 [SMILES: Cc1cc(NC(=O)N2CCC[C@@H]2C(=O)Nc2ccc(-c3ccc(C(=O)O)nc3)cc2)ccc1C(F)(F)F]

### Step 1. Synthesis of tert-butyl (2R)-2-{[4-(4,4,5,5-tetramethyl-1,3,2-dioxaborolan-2-yl)phenyl]carbamoyl}pyrrolidine-1-carboxylate (C19)

To a mixture of 4-(4,4,5,5-tetramethyl-1,3,2-dioxaborolan-2-yl)aniline (4.00 g, 18.3 mmol) and 1-(tert-butoxycarbonyl)-D-proline (3.93 g, 18.3 mmol) in dichloromethane (90 mL) was added 1-[3-(dimethylamino)propyl]-3-ethylcarbodiimide hydrochloride (4.55 g, 23.7 mmol) in one portion. After the reaction mixture had been stirred for 18 hours at room temperature, it was diluted with dichloromethane, washed with water, dried over magnesium sulfate, filtered, and concentrated in vacuo. The residue was triturated with diethyl ether to afford C19 as a white solid. By ¹H NMR analysis, this material exists as a mixture of rotamers at room temperature. Yield: 6.82 g, 16.4 mmol, 90%. LCMS m/z 417.4 [M+H]⁺. ¹H NMR (400 MHz, DMSO-d₆, 25° C.) δ 10.09 (s, 1H), 7.64-7.59 (m, 4H), [4.26 (br dd, J=8.4, 3.2 Hz) and 4.19 (dd, J=8.2, 4.5 Hz), total 1H], 3.46-3.37 (m, 1H), 3.37-3.3 (m, 1H, assumed; partially obscured by water peak), 2.25-2.10 (m, 1H), 1.96-1.72 (m, 3H), 1.39 (s, 3H), 1.28 (s, 12H), 1.25 (s, 6H).

¹H NMR (400 MHz, DMSO-d₆, 60° C.) δ 9.91 (s, 1H), 7.61 (s, 4H), 4.32-4.17 (m, 1H), 3.48-3.40 (m, 1H), 3.40-3.31 (m, 1H), 2.27-2.12 (m, 1H), 1.98-1.74 (m, 3H), 1.50-1.17 (m, 21H).

### Step 2. Synthesis of tert-butyl 5-(4-{[1-(tert-butoxycarbonyl)-D-prolyl]amino}phenyl)pyridine-2-carboxylate (C20)

A mixture of tert-butyl 5-bromopyridine-2-carboxylate (744 mg, 2.88 mmol), C19 (1.00 g, 2.40 mmol), sodium carbonate (764 mg, 7.21 mmol), and tetrakis(triphenylphosphine)palladium(0) (208 mg, 0.180 mmol) was treated with 1,2-dimethoxyethane (12 mL) and water (3 mL), whereupon the reaction vessel was evacuated and charged with nitrogen. After this evacuation cycle had been repeated twice, the reaction mixture was heated at 100° C. for 18 hours, cooled to room temperature, and filtered. The filter cake was washed with diethyl ether and ethyl acetate, then triturated with methanol to provide C20 as a white solid (634 mg). The filtrate was diluted with water and ethyl acetate, and the aqueous layer was extracted twice with ethyl acetate. The combined organic layers were dried over magnesium sulfate, filtered, and concentrated in vacuo; the resulting material was triturated with methanol, affording C20 as a white solid (92 mg). The two lots of product were combined and concentrated three times from dichloromethane, providing C20 as a white solid. Yield: 700 mg, 1.50 mmol, 62%. LCMS m/z 468.4 [M+H]⁺. ¹H NMR (400 MHz, DMSO-d₆) δ 10.20 (s, 1H), 9.01 (br s, 1H), 8.22 (dd, component of ABX system, J=8.2, 2.4 Hz, 1H), 8.03 (d, half of AB quartet, J=8.2 Hz, 1H), 7.78 (s, 4H), [4.29 (br dd, J=8.5, 3.1 Hz) and 4.22 (dd, J=8.3, 4.4 Hz), total 1H], 3.48-3.39 (m, 1H), 3.39-3.3 (m, 1H, assumed; partially obscured by water peak), 2.29-2.13 (m, 1H), 1.98-1.74 (m, 3H), 1.58 (s, 9H), 1.40 (s, 3H), 1.28 (s, 6H).

### Step 3. Synthesis of 5-[4-(D-prolylamino)phenyl]pyridine-2-carboxylic acid, dihydrochloride salt (C21)

To a suspension of C20 (700 mg, 1.50 mmol) in 1,4-dioxane (4 mL) was added a solution of hydrogen chloride in 1,4-dioxane (4 M; 3.74 mL, 15.0 mmol). After the reaction mixture had been stirred for 17 hours, LCMS analysis indicated conversion to C21: LCMS m/z 312.2 [M+H]⁺. Filtration afforded a filter cake, which was washed with 1,4-dioxane to provide C21 as a yellow solid (628 mg). A portion of this material was progressed to the following step. ¹H NMR (400 MHz, DMSO-d₆) δ 11.12 (s, 1H), 10.01-9.87 (m, 1H), 9.03 (br d, J=2 Hz, 1H), 8.77-8.66 (m, 1H), 8.27 (dd, component of ABX system, J=8.2, 2.4 Hz, 1H), 8.11 (d, half of AB quartet, J=8.2 Hz, 1H), 7.84 (AB quartet, J_{AB}=8.8 Hz, Δv_{AB}=15.4 Hz, 4H), 4.50-4.38 (m, 1H), 3.36-3.19 (m, 2H), 2.5-2.38 (m, 1H, assumed; partially obscured by solvent peak), 2.05-1.89 (m, 3H).

### Step 4. Synthesis of 5-{4-[(1-{[3-methyl-4-(trifluoromethyl)phenyl]carbamoyl}-D-prolyl)amino]phenyl}pyridine-2-carboxylic acid (17)

To a solution of 3-methyl-4-(trifluoromethyl)aniline (19.4 mg, 0.111 mmol) in dichloromethane (1 mL) was added a solution of bis(trichloromethyl) carbonate (11.5 mg, 38.8 μmol) in dichloromethane (0.5 mL), followed by 4-(dimethylamino)pyridine (67.8 mg, 0.555 mmol). After the reaction mixture had been stirred for 1 hour at room temperature, C21 (from the previous step; 38 mg, ≤91 μmol) was added in one portion, and stirring was continued for 45 minutes. Dichloromethane and water were added, whereupon the pH was adjusted to 4 to 5 by addition of 1 M hydrochloric acid. The organic layer was concentrated to dryness, azeotroped twice with dichloromethane, and purified via reversed-phase HPLC (Column: Waters Sunfire C18, 19×100 mm, 5 μm; Mobile phase A: 0.05% trifluoroacetic acid in water (v/v); Mobile phase B: 0.05% trifluoroacetic acid in acetonitrile (v/v); Gradient: 20% to 60% B over 8.5 minutes, then 60% to 95% B over 0.5 minutes, then 95% B for 1.0 minute; Flow rate: 25 mL/minute) to afford 5-{4-[(1-{[3-methyl-4-(trifluoromethyl)phenyl]carbamoyl}-D-prolyl)amino]phenyl}pyridine-2-carboxylic acid (17). Yield: 10.2 mg, 19.9 μmol, 22% over 2 steps. LCMS m/z 513.3 [M+H]⁺. ¹H NMR (600 MHz, DMSO-d₆) δ 10.22 (s, 1H), 9.01 (d, J=2.3 Hz, 1H), 8.61 (s, 1H), 8.24 (dd, component of ABX system, J=8.2, 2.4 Hz, 1H), 8.09 (br d, half of AB quartet, J=8.2 Hz, 1H), 7.79 (AB quartet, J_{AB}=9.0 Hz, Δv_{AB}=11.1 Hz, 4H), 7.61 (br s, 1H), 7.53 (AB quartet, downfield doublet is broadened, J_{AB}=8.8 Hz, Δv_{AB}=23.8 Hz, 2H), 4.49 (dd, J=8.3, 3.9 Hz, 1H), 3.70-3.64 (m, 1H), 3.59-3.53 (m, 1H), 2.36 (br s, 3H), 2.27-2.18 (m, 1H), 2.08-1.90 (m, 3H).

### Example 18

### 4-{5-[(1-{[4-(Propan-2-yl)phenyl]carbamoyl}-D-prolyl)amino]pyridin-2-yl}benzoic acid (18)

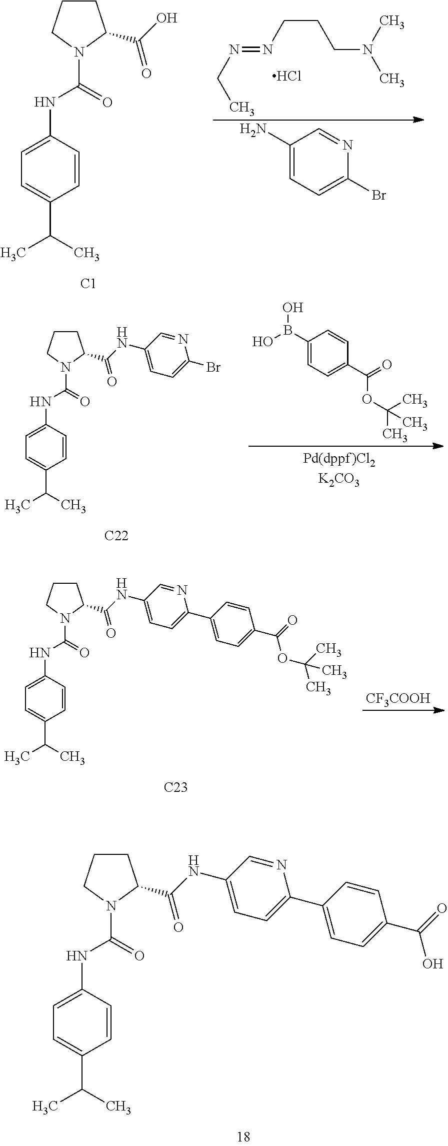

### Step 1. Synthesis of (2R)—N²-(6-bromopyridin-3-yl)-N¹-[4-(propan-2-yl)phenyl]pyrrolidine-1,2-dicarboxamide (C22)

1-[3-(Dimethylamino)propyl]-3-ethylcarbodiimide hydrochloride (166 mg, 0.866 mmol) was added in a single portion to a 0° C. solution of C1 (200 mg, 0.724 mmol) in dichloromethane (3.6 mL. The reaction mixture was stirred at 0° C. for 30 minutes, whereupon 6-bromopyridin-3-amine (125 mg, 0.723 mmol) was added, and stirring was continued at 0° C. for 4 hours. The reaction mixture was poured into water and the resulting mixture was extracted twice with dichloromethane; the combined organic layers were washed sequentially with water and saturated aqueous sodium chloride solution, dried over sodium sulfate, filtered, and concentrated in vacuo to provide C22 as a light-orange solid. Yield: 320 mg, assumed quantitative. LCMS m/z 431.3 (bromine isotope pattern observed) [M+H]⁺. ¹H NMR (600 MHz, DMSO-d₆) δ 10.35 (s, 1H), 8.62 (d, J=2.8 Hz, 1H), 8.21 (s, 1H), 7.99 (dd, J=8.7, 2.8 Hz, 1H), 7.58 (d, J=8.7 Hz, 1H), 7.38 (d, J=8.5 Hz, 2H), 7.08 (d, J=8.5 Hz, 2H), 4.43 (dd, J=8.4, 3.8 Hz, 1H), 3.66-3.60 (m, 1H), 3.54-3.47 (m, 1H), 2.80 (septet, J=6.9 Hz, 1H), 2.23-2.14 (m, 1H), 2.05-1.88 (m, 3H), 1.16 (d, J=6.9 Hz, 6H).

### Step 2. Synthesis of tert-butyl 4-{5-[(1-{[4-(propan-2-yl)phenyl]carbamoyl}-D-prolyl)amino]pyridin-2-yl}benzoate (C23)

A mixture of [4-(tert-butoxycarbonyl)phenyl]boronic acid (34.0 mg, 0.153 mmol), C22 (60.0 mg, 0.139 mmol), potassium carbonate (57.7 mg, 0.418 mmol), and [1,1′-bis(diphenylphosphino)ferrocene]dichloropalladium(II) (5.09 mg, 6.96 μmol) in a mixture of 1,4-dioxane (0.7 mL), and water (0.1 mL) was sparged three times with nitrogen, whereupon it was heated overnight at 60° C. The temperature was then increased to 80° C.; after 2.5 hours, the reaction mixture was allowed to cool to room temperature and filtered through a plug of diatomaceous earth. The filtrate was partitioned between ethyl acetate and water, and the aqueous layer was extracted twice with ethyl acetate. The combined organic layers were washed sequentially with water and saturated aqueous sodium chloride solution, dried over magnesium sulfate, filtered, and concentrated in vacuo to afford C23 as an orange oil (76.4 mg); the bulk of this material was progressed to the following step. LCMS m/z 529.5 [M+H]⁺.

### Step 3. Synthesis of 4-{5-[(1-{[4-(propan-2-yl)phenyl]carbamoyl}-D-prolyl)amino]pyridin-2-yl}benzoic acid (18)

Trifluoroacetic acid (0.2 mL) was added drop-wise to a solution of C23 (from the previous step; 74.0 mg, ≤0.135 mmol) in dichloromethane (1.0 mL). The reaction mixture was stirred at room temperature for 3.75 hours, whereupon it was concentrated in vacuo. Purification of the residue via reversed-phase HPLC (Column: Waters Sunfire C18, 19×100 mm, 5 μm; Mobile phase A: 0.05% trifluoroacetic acid in water (v/v); Mobile phase B: 0.05% trifluoroacetic acid in acetonitrile (v/v); Gradient: 20% to 60% B over 8.5 minutes, then 60% to 95% B over 0.5 minutes, then 95% B for 1.0 minute; Flow rate: 25 mL/minute) provided 4-{5-[(1-{[4-(propan-2-yl)phenyl]carbamoyl}-D-prolyl)amino]pyridin-2-yl}benzoic acid (18). Yield: 16.4 mg, 34.7 μmol, 26% over 2 steps. LCMS m/z 473.4 [M+H]⁺. ¹H NMR (600 MHz, DMSO-d₆) δ 10.40 (s, 1H), 8.89 (d, J=2.4 Hz, 1H), 8.23 (s, 1H), 8.20 (dd, J=8.7, 2.4 Hz, 1H), 8.17 (d, J=8.4 Hz, 2H), 8.06-8.01 (m, 3H), 7.40 (br d, J=8.3 Hz, 2H), 7.09 (d, J=8.6 Hz, 2H), 4.49 (dd, J=8.4, 3.8 Hz, 1H), 3.69-3.63 (m, 1H), 3.55-3.49 (m, 1H, assumed; partially obscured by water peak), 2.80 (septet, J=6.9 Hz, 1H), 2.26-2.18 (m, 1H), 2.09-1.92 (m, 3H), 1.16 (d, J=6.9 Hz, 6H).

### Alternate Synthesis of Example 18

### 4-{5-[(1-{[4-(Propan-2-yl)phenyl]carbamoyl}-D-prolyl)amino]pyridin-2-yl}benzoic acid (18)

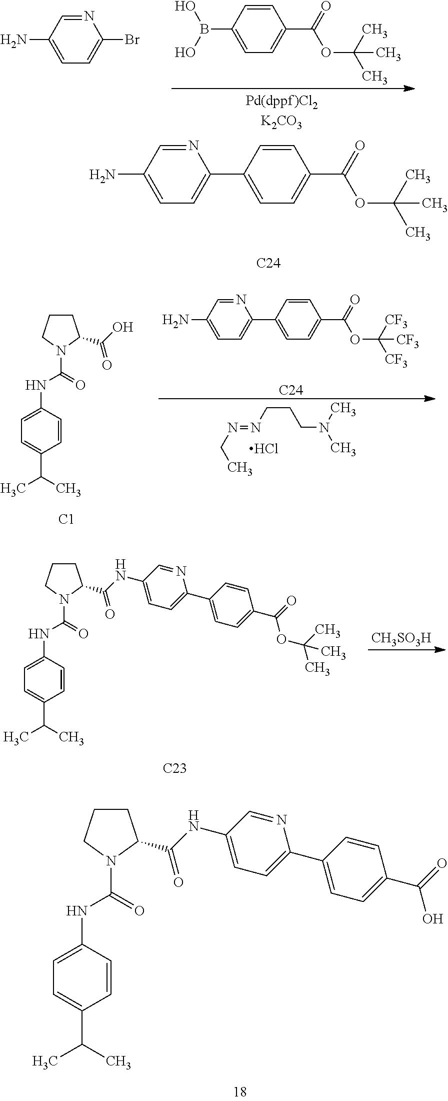

### Step 1. Synthesis of tert-butyl 4-(5-aminopyridin-2-yl)benzoate (C24)

A solution of [4-(tert-butoxycarbonyl)phenyl]boronic acid (750 mg, 3.38 mmol), 6-bromopyridin-3-amine (643 mg, 3.72 mmol), and potassium carbonate (1.40 g, 10.1 mmol) in a mixture of 1,4-dioxane (30 mL) and water (15 mL) was sparged with nitrogen for 10 minutes, whereupon [1,1′-bis(diphenylphosphino)ferrocene]dichloropalladium(II) (124 mg, 0.169 mmol) was added. After the reaction mixture had been heated at 95° C. for 3 hours, it was partitioned between ethyl acetate and water. The aqueous layer was extracted with ethyl acetate (5×20 mL), and the combined organic layers were washed with saturated aqueous sodium chloride solution, dried over sodium sulfate, filtered, and concentrated in vacuo. LCMS m/z 271.3 [M+H]⁺. Purification via silica gel chromatography (Gradient: 0% to 40% ethyl acetate in heptane) afforded C24 as a white powder. Yield: 669 mg, 2.47 mmol, 73%. ¹H NMR (400 MHz, DMSO-d₆) δ 8.05 (d, J=2.8 Hz, 1H), 8.03 (d, J=8.6 Hz, 2H), 7.90 (d, J=8.5 Hz, 2H), 7.73 (d, J=8.6 Hz, 1H), 7.00 (dd, J=8.6, 2.8 Hz, 1H), 5.64 (br s, 2H), 1.56 (s, 9H7).

### Step 2. Synthesis of tert-butyl 4-{5-[(1-{[4-(propan-2-yl)phenyl]carbamoyl}-D-prolyl)amino]pyridin-2-yl}benzoate (C23)

1-[3-(Dimethylamino)propyl]-3-ethylcarbodiimide hydrochloride (361 mg, 1.88 mmol) was added to a solution of C1 (400 mg, 1.45 mmol) and C24 (391 mg, 1.45 mmol) in dichloromethane (7 mL). After the reaction mixture had been stirred for 30 minutes, LCMS analysis indicated formation of C23: LCMS m/z 529.5 [M+H]⁺. The reaction mixture was diluted with dichloromethane, washed sequentially with water and saturated aqueous sodium bicarbonate solution, dried over sodium sulfate, filtered, and concentrated in vacuo. Silica gel chromatography (Gradient: 0% to 75% ethyl acetate in heptane) provided C23 as a white solid. Yield: 492 mg, 0.931 mmol, 64%. ¹H NMR (400 MHz, DMSO-d₆) δ 10.38 (s, 1H), 8.88 (d, J=2.5 Hz, 1H), 8.24-8.18 (m, 2H), 8.16 (d, J=8.5 Hz, 2H), 8.03 (d, J=8.7 Hz, 1H), 7.98 (d, J=8.5 Hz, 2H), 7.40 (d, J=8.6 Hz, 2H), 7.09 (d, J=8.6 Hz, 2H), 4.49 (dd, J=8.4, 3.6 Hz, 1H), 3.70-3.61 (m, 1H), 3.57-3.48 (m, 1H), 2.80 (septet, J=6.9 Hz, 1H), 2.27-2.15 (m, 1H), 2.11-1.89 (m, 3H), 1.57 (s, 9H), 1.16 (d, J=6.9 Hz, 6H).

### Step 3. Synthesis of 4-{5-[(1-{[4-(propan-2-yl)phenyl]carbamoyl}-D-prolyl)amino]pyridin-2-yl}benzoic acid (18)

Methanesulfonic acid (0.442 mL, 6.81 mmol) was added to a mixture of C23 (300 mg, 0.567 mmol) in acetonitrile (2.8 mL). After the reaction mixture had been stirred for one hour, LCMS analysis indicated complete cleavage of the ester: LCMS m/z 473.4 [M+H]⁺. The reaction mixture was concentrated in vacuo to remove half of the acetonitrile, whereupon water (10 mL) was added. The resulting mixture was adjusted to pH 7 to 8 by addition of saturated aqueous sodium bicarbonate solution, then allowed to stir for 10 minutes. Solids were isolated via filtration and washed with water to provide 4-{5-[(1-{[4-(propan-2-yl)phenyl]carbamoyl}-D-prolyl)amino]pyridin-2-yl}benzoic acid (18) as an off-white solid. Yield: 162 mg, 0.343 mmol, 60%. ¹H NMR (400 MHz, DMSO-d₆) δ 12.98 (br s, 1H), 10.37 (s, 1H), 8.88 (d, J=2.5 Hz, 1H), 8.24-8.17 (m, 2H), 8.17 (d, J=8.5 Hz, 2H), 8.03 (d, J=8.4 Hz, 1H), 8.02 (d, J=8.5 Hz, 2H), 7.40 (d, J=8.6 Hz, 2H), 7.09 (d, J=8.6 Hz, 2H), 4.49 (dd, J=8.5, 3.6 Hz, 1H), 3.71-3.61 (m, 1H), 3.58-3.48 (m, 1H), 2.79 (septet, J=6.9 Hz, 1H), 2.27-2.15 (m, 1H), 2.11-1.89 (m, 3H), 1.16 (d, J=6.9 Hz, 6H).

### Example 19

### 5-{4-[(1-{[3-Methyl-4-(propan-2-yl)phenyl]carbamoyl}-D-prolyl)amino]phenyl}pyridine-2-carboxylic acid (19)

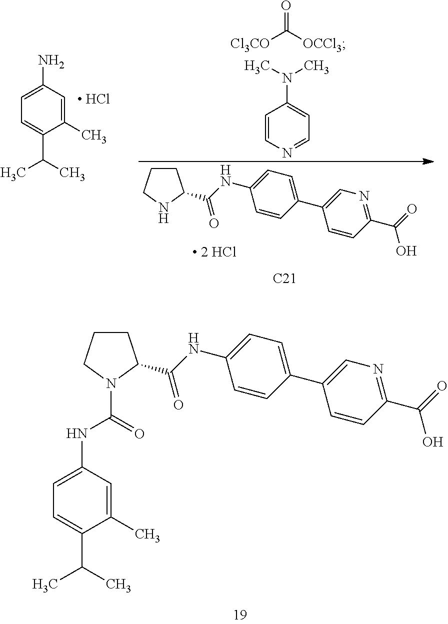

To a solution of 3-methyl-4-(propan-2-yl)aniline, hydrochloride salt (20.6 mg, 0.111 mmol) in dichloromethane (1 mL) was added a solution of bis(trichloromethyl) carbonate (11.5 mg, 38.8 μmol) in dichloromethane (0.5 mL), followed by 4-(dimethylamino)pyridine (67.8 mg, 0.555 mmol). After the reaction mixture had been stirred for 1 hour at room temperature, C21 (from Example 17, Step 3; 38 mg, ≤91 μmol) was added in one portion, and stirring was continued for 45 minutes. The reaction mixture was then diluted with dichloromethane and water, and the pH was adjusted to approximately 5 by addition of 1 M hydrochloric acid. The organic layer was collected using a pipette, and the remaining aqueous mixture was filtered; residues in the reaction vessel and on the filter paper were dissolved in a 1:1 mixture of dichloromethane and methanol, which was added to the organic layer. This solution was concentrated in vacuo, azeotroped twice with dichloromethane, and purified twice via reversed-phase HPLC (Purification #1. Column: Waters Sunfire C18, 19×100 mm, 5 μm; Mobile phase A: 0.05% trifluoroacetic acid in water (v/v); Mobile phase B: 0.05% trifluoroacetic acid in acetonitrile (v/v); Gradient: 15% to 55% B over 8.5 minutes, then 55% to 95% B over 0.5 minutes, then 95% B for 1.0 minute; Flow rate: 25 mL/minute. Purification #2. Column: Waters Sunfire C18, 19×100 mm, 5 μm; Mobile phase A: 0.05% trifluoroacetic acid in water (v/v); Mobile phase B: 0.05% trifluoroacetic acid in acetonitrile (v/v); Gradient: 20% to 60% B over 8.5 minutes, then 60% to 95% B over 0.5 minutes, then 95% B for 1.0 minute; Flow rate: 25 mL/minute) to afford 5-{4-[(1-{[3-methyl-4-(propan-2-yl)phenyl]carbamoyl}-D-prolyl)amino]phenyl}pyridine-2-carboxylic acid (19). Yield: 14.1 mg, 29.0 μmol, 32% over 2 steps. LCMS m/z 487.5 [M+H]⁺. ¹H NMR (600 MHz, DMSO-d₆), characteristic peaks: δ 10.19 (s, 1H), 9.02-8.98 (m, 1H), 8.24 (dd, component of ABX system, J=8.2, 2.4 Hz, 1H), 8.12 (s, 1H), 8.08 (br d, J=8.2 Hz, 1H), 7.78 (AB quartet, J_{AB}=9.0 Hz, Δv_{AB}=10.1 Hz, 4H), 7.28-7.24 (m, 2H), 7.06 (d, J=8.2 Hz, 1H), 4.46 (dd, J=8.3, 3.8 Hz, 1H), 3.66-3.60 (m, 1H), 3.01 (septet, J=6.9 Hz, 1H), 2.22 (s, 3H), 2.22-2.15 (m, 1H), 2.07-1.98 (m, 1H), 1.98-1.88 (m, 2H), 1.13 (d, J=6.8 Hz, 6H).

### Example 20

### 6-Methyl-5-{4-[(1-{[4-(propan-2-yl)phenyl]carbamoyl}-D-prolyl)amino]phenyl}pyridine-2-carboxylic acid (20)

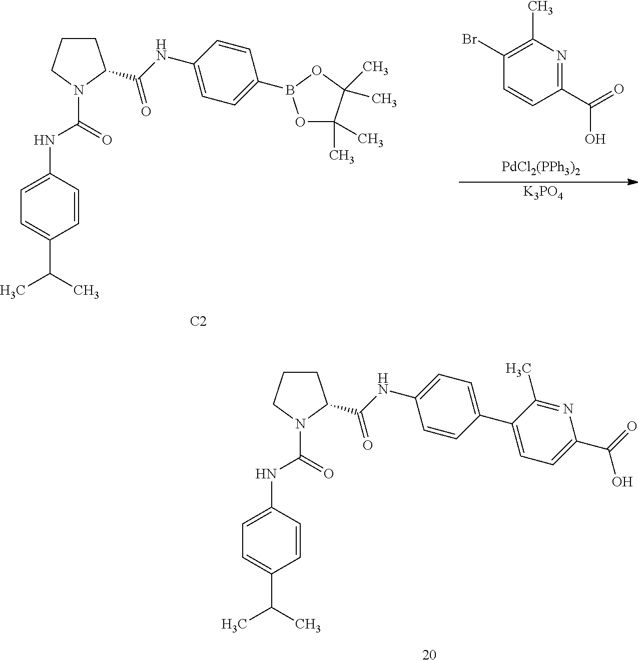

A 1.5 M aqueous solution of tripotassium phosphate was prepared and sparged with nitrogen. A solution of C2 (40.0 mg, 83.8 μmol), 5-bromo-6-methylpyridine-2-carboxylic acid (19.9 mg, 92.1 μmol), and bis(triphenylphosphine)palladium(II) dichloride (5.88 mg, 8.38 μmol) in 1,4-dioxane (1.0 mL) was sparged with nitrogen for 10 minutes, whereupon it was heated at 50° C. After 5 minutes, the aqueous solution of tripotassium phosphate (1.5 M; 0.168 mL, 0.252 mmol) was added; the reaction mixture was heated at 90° C. overnight, whereupon it was cooled to room temperature. Solvent was removed using a stream of nitrogen, and the residue was taken up in a 9:1 mixture of dichloromethane and methanol. This was acidified via addition of 1 M hydrochloric acid, and the organic layer was concentrated in vacuo. Purification via reversed-phase HPLC (Column: Waters Sunfire C18, 19×100 mm, 5 μm; Mobile phase A: 0.05% trifluoroacetic acid in water (v/v); Mobile phase B: 0.05% trifluoroacetic acid in acetonitrile (v/v); Gradient: 15% to 55% B over 8.5 minutes, then 55% to 95% B over 0.5 minutes, then 95% B for 1.0 minute; Flow rate: 25 mL/minute) afforded 6-methyl-5-{4-[(1-{[4-(propan-2-yl)phenyl]carbamoyl}-D-prolyl)amino]phenyl}pyridine-2-carboxylic acid (20). Yield: 19.3 mg, 39.7 μmol, 47%. LCMS m/z 487.5 [M+H]⁺. ¹H NMR (600 MHz, DMSO-d₆), characteristic peaks: δ 10.16 (s, 1H), 8.20 (s, 1H), 7.93 (d, J=7.9 Hz, 1H), 7.77 (d, J=7.9 Hz, 1H), 7.74 (d, J=8.6 Hz, 2H), 7.41-7.37 (m, 4H), 7.09 (d, J=8.6 Hz, 2H), 4.47 (dd, J=8.3, 3.8 Hz, 1H), 3.67-3.62 (m, 1H), 2.80 (septet, J=7.0 Hz, 1H), 2.51 (s, 3H, assumed; partially obscured by solvent peak), 2.24-2.15 (m, 1H), 2.08-1.99 (m, 1H), 1.99-1.90 (m, 2H), 1.16 (d, J=6.9 Hz, 6H).

### Alternate Synthesis of Example 20

### 6-Methyl-5-{4-[(1-{[4-(propan-2-yl)phenyl]carbamoyl}-D-prolyl)amino]phenyl}pyridine-2-carboxylic acid (20)

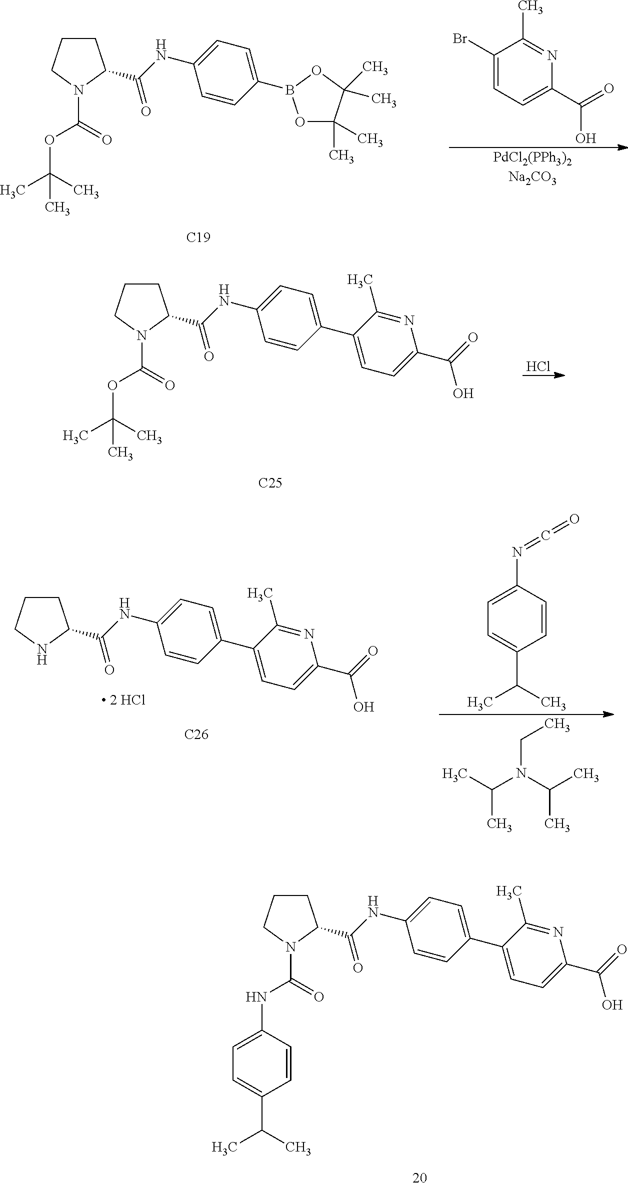

### Step 1. Synthesis of 5-(4-{[1-(tert-butoxycarbonyl)-D-prolyl]amino}phenyl)-6-methylpyridine-2-carboxylic acid (C25)

To a solution of C19 (3.00 g, 7.21 mmol) and 5-bromo-6-methylpyridine-2-carboxylic acid (1.71 g, 7.92 mmol) in 1,4-dioxane (50 mL) was added an aqueous solution of sodium carbonate (2.0 M; 14.4 mL, 28.8 mmol), followed by bis(triphenylphosphine)palladium(II) dichloride (253 mg, 0.360 mmol). The reaction mixture was degassed for 5 minutes, whereupon it was heated at 90° C. overnight. After cooling to room temperature, the reaction mixture was partitioned between ethyl acetate (100 mL) and water (50 mL), and the aqueous layer was adjusted to pH 4 by addition of 3 M hydrochloric acid. The resulting mixture was extracted with ethyl acetate (3×200 mL); the combined organic extracts were washed with saturated aqueous sodium chloride solution, dried over magnesium sulfate, filtered, and concentrated in vacuo, affording C25 as a yellow solid. By ¹H NMR analysis, this material exists as a mixture of rotamers. Yield: 2.95 g, 6.93 mmol, 96%. LCMS m/z 426.4 [M+H]⁺. ¹H NMR (400 MHz, methanol-d₄) δ 8.06 (d, J=7.9 Hz, 1H), 7.87 (d, J=7.8 Hz, 1H), 7.74 (br d, J=8 Hz, 2H), 7.44-7.33 (m, 2H), [4.41-4.34 (m) and 4.30 (dd, J=8.2, 4.7 Hz), total 1H], 3.62-3.53 (m, 1H), 3.53-3.43 (m, 1H), 2.58 (s, 3H), 2.41-2.22 (m, 1H), 2.11-1.97 (m, 2H), 1.97-1.85 (m, 1H), [1.48 (s) and 1.39 (s), total 9H].

### Step 2. Synthesis of 6-methyl-5-[4-(D-prolylamino)phenyl]pyridine-2-carboxylic acid, dihydrochloride salt (C26)

A solution of hydrogen chloride in 1,4-dioxane (4.0 M; 6.93 mL, 27.7 mmol) was added to a solution of C25 (2.95 g, 6.93 mmol) in a mixture of dichloromethane (40 mL) and 1,1,1,3,3,3-hexafluoropropan-2-ol (5 mL). The reaction mixture was stirred at room temperature for 18 hours, whereupon LCMS analysis indicated conversion to C26: LCMS m/z 326.3 [M+H]⁺. Collection of the precipitate via filtration provided C26 as a yellow solid. Yield: 2.55 g, 6.40 mmol, 92%. ¹H NMR (400 MHz, DMSO-d₆) δ 11.20 (s, 1H), 10.17-10.04 (m, 1H), 8.77-8.65 (m, 1H), 7.93 (AB quartet, J_{AB}=7.9 Hz, Δv_{AB}=46.6 Hz, 2H), 7.80 (d, J=8.6 Hz, 2H), 7.47 (d, J=8.6 Hz, 2H), 4.51-4.39 (m, 1H), 3.37-3.19 (m, 2H), 2.54 (s, 3H), 2.5-2.41 (m, 1H, assumed; partially obscured by solvent peak), 2.06-1.88 (m, 3H).

### Step 3. Synthesis of 6-methyl-5-{4-[(1-{[4-(propan-2-yl)phenyl]carbamoyl}-D-prolyl)amino]phenyl}pyridine-2-carboxylic acid (20)

A solution of C26 (320 mg, 0.803 mmol) and N,N-diisopropylethylamine (280 μL, 1.61 mmol) in acetonitrile (5 mL) was stirred at 22° C. for 30 minutes, whereupon it was added to 1-isocyanato-4-(propan-2-yl)benzene (155 mg, 0.962 mmol), and the reaction mixture was allowed to stir at room temperature for 5 hours. Water (25 mL) was added, and the resulting mixture was adjusted to pH 5 by addition of 1 M hydrochloric acid. The aqueous layer was extracted with ethyl acetate (2×40 mL), and the combined organic layers were concentrated in vacuo and purified via silica gel chromatography (Gradient: 0% to 10% methanol in dichloromethane). The resulting material (250 mg) was treated with ethyl acetate (3 mL) and heated until a solution was obtained, whereupon heptane was added until the mixture became cloudy. This was stirred at room temperature for 18 hours, concentrated in vacuo, and treated with propan-2-yl acetate (2 mL), heated to dissolution, and slowly cooled to room temperature. Heptane was added and the mixture was stirred for 1 hour, whereupon the solid was collected via filtration to afford 6-methyl-5-{4-[(1-{[4-(propan-2-yl)phenyl]carbamoyl}-D-prolyl)amino]phenyl}pyridine-2-carboxylic acid (20) as a white solid. Yield: 155 mg, 0.319 mmol, 40%. LCMS m/z 487.4 [M+H]⁺. ¹H NMR (400 MHz, DMSO-d₆) δ 10.15 (s, 1H), 8.19 (s, 1H), 7.92 (d, J=7.9 Hz, 1H), 7.75 (d, J=7.7 Hz, 1H), 7.74 (d, J=8.5 Hz, 2H), 7.40 (d, J=8.4 Hz, 2H), 7.39 (d, J=8.4 Hz, 2H), 7.09 (d, J=8.5 Hz, 2H), 4.48 (dd, J=8.3, 3.6 Hz, 1H), 3.70-3.60 (m, 1H), 3.56-3.46 (m, 1H), 2.80 (septet, J=6.9 Hz, 1H), 2.5 (s, 3H, assumed; overlaps with solvent peak), 2.26-2.13 (m, 1H), 2.10-1.88 (m, 3H), 1.16 (d, J=6.9 Hz, 6H).

### Example 21

### 3-Methoxy-4′-[(1-{[4-(propan-2-yl)phenyl]carbamoyl}-D-prolyl)amino][1,1′-biphenyl]-4-carboxylic acid (21)

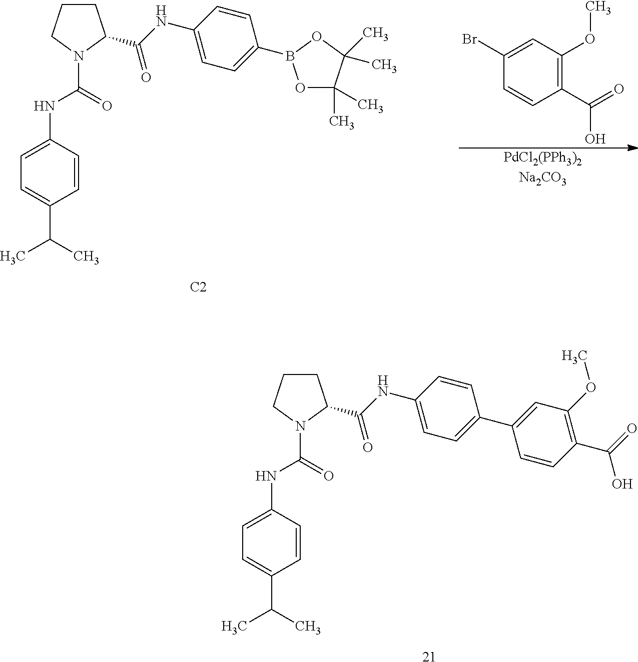 [SMILES: CC(C)c1ccc(NC(=O)N2CCC[C@@H]2C(=O)Nc2ccc(B3OC(C)(C)C(C)(C)O3)cc2)cc1 ; COc1cc(Br)ccc1C(=O)O ; "C2" ; COc1cc(-c2ccc(NC(=O)[C@H]3CCCN3C(=O)Nc3ccc(C(C)C)cc3)cc2)ccc1C(=O)O ; "21" ; "PdCl2(PPh3)2Na2CO3"]

To a mixture of C2 (400 mg, 0.838 mmol) and 4-bromo-2-methoxybenzoic acid (194 mg, 0.840 mmol) in 1,4-dioxane (6 mL) was added aqueous sodium carbonate solution (2.0 M; 1.68 mL, 3.36 mmol), followed by bis(triphenylphosphine)palladium(II) dichloride (47.0 mg, 67 μmol). After the reaction mixture had been degassed for 5 minutes, it was heated at 90° C. overnight, whereupon it was cooled to room temperature and adjusted to pH 5 by addition of 1 M hydrochloric acid. The resulting mixture was partitioned between ethyl acetate (50 mL) and water (10 mL); the organic layer was then washed sequentially with water (2×10 mL) and saturated aqueous sodium chloride solution, dried over magnesium sulfate, filtered, and concentrated in vacuo. Chromatography on silica gel (Gradient: 0% to 10% methanol in dichloromethane) provided a solid, which was heated at 70° C. for 10 minutes in ethyl acetate containing a few drops of methanol. The mixture was then stirred at room temperature for 20 hours. Solids were collected via filtration, washed with diethyl ether, and subjected once more to silica gel chromatography (Gradient: 0% to 40% ethyl acetate in dichloromethane), affording 3-methoxy-4′-[(1-{[4-(propan-2-yl)phenyl]carbamoyl}-D-prolyl)amino][1,1′-biphenyl]-4-carboxylic acid (21) as a white solid. Yield: 100 mg, 0.199 mmol, 24%. LCMS m/z 502.4 [M+H]⁺. ¹H NMR (400 MHz, methanol-d₄) δ 7.91 (d, J=8.1 Hz, 1H), 7.69 (AB quartet, J_{AB}=8.8 Hz, Δv_{AB}=17.9 Hz, 4H), 7.34-7.30 (m, 3H), 7.28 (dd, J=8.1, 1.6 Hz, 1H), 7.14 (d, J=8.5 Hz, 2H), 4.59 (dd, J=8.2, 3.4 Hz, 1H), 4.00 (s, 3H), 3.78-3.70 (m, 1H), 3.64-3.55 (m, 1H), 2.85 (septet, J=6.9 Hz, 1H), 2.38-2.24 (m, 1H), 2.23-2.03 (m, 3H), 1.21 (d, J=6.9 Hz, 6H).

### Alternate Synthesis of Example 21

### 3-Methoxy-4′-[(1-{[4-(propan-2-yl)phenyl]carbamoyl}-D-prolyl)amino][1,1′-biphenyl]-4-carboxylic acid (21)

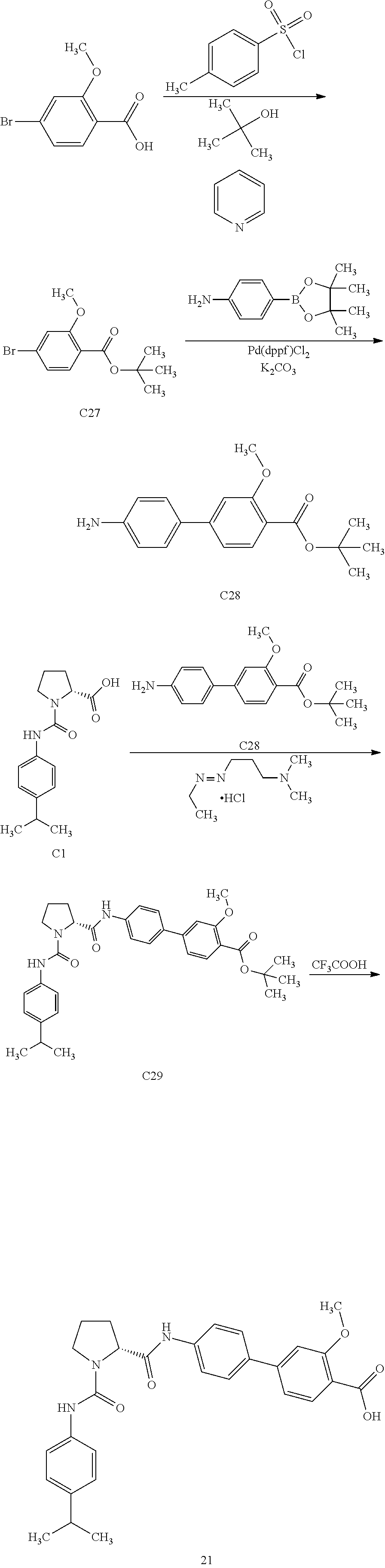

### Step 1. Synthesis of tert-butyl 4-bromo-2-methoxybenzoate (C27)

To a solution of 4-bromo-2-methoxybenzoic acid (40 g, 170 mmol) in tert-butanol (300 mL) were added pyridine (600 mL) and 4-methylbenzene-1-sulfonyl chloride (99 g, 520 mmol). The resulting mixture was allowed to stir at room temperature for 2 days, whereupon water (1 L) was added to the reaction mixture, followed by solid sodium hydroxide (3.0 g, 75 mmol). The resulting mixture had an approximate pH of 7 to 8. The mixture was extracted with ethyl acetate (2×1 L), and the combined organic layers were washed with saturated aqueous sodium chloride solution (3×500 mL), dried over sodium sulfate, filtered, and concentrated in vacuo, affording C27 as a yellow oil (50 g). This material was progressed directly to the following step. ¹H NMR (400 MHz, chloroform-d) δ 7.59 (d, J=8.7 Hz, 1H), 7.09-7.05 (m, 2H, assumed, partially obscured by solvent peak), 3.87 (s, 3H), 1.56 (s, 9H).

### Step 2. Synthesis of tert-butyl 4′-amino-3-methoxy[1,1′-biphenyl]-4-carboxylate (C28)

To a solution of 4-(4,4,5,5-tetramethyl-1,3,2-dioxaborolan-2-yl)aniline (38.1 g 174 mmol) in a mixture of 1,4-dioxane (800 mL) and water (160 mL) were added C27 (from the previous step; 50 g, 5170 mmol), potassium carbonate (48.1 g, 348 mmol), and [1,1′-bis(diphenylphosphino)ferrocene]dichloropalladium(II) (6.37 g, 8.71 mmol). After the reaction mixture had been heated at 90° C. for 2 hours, it was filtered, and the filtrate was concentrated under reduced pressure. The aqueous residue was diluted with ethyl acetate (1 L), washed sequentially with water (1 L) and saturated aqueous sodium chloride solution (2×1 L), dried over sodium sulfate, filtered, and concentrated in vacuo. Chromatography on silica gel (Gradient: 0% to 50% ethyl acetate in petroleum ether) provided a solid, which was stirred with a mixture of petroleum ether and ethyl acetate (5:1, 60 mL) for 30 minutes, then filtered. The filter cake was washed with a mixture of petroleum ether and ethyl acetate (5:1, 30 mL) to afford C28 as a yellow solid. Yield: 42.0 g, 140 mmol, 82% over 2 steps. LCMS m/z 300.3 [M+H]⁺. ¹H NMR (400 MHz, chloroform-d) δ 7.79 (d, J=8.0 Hz, 1H), 7.43 (d, J=8.5 Hz, 2H), 7.11 (dd, component of ABX system, J=8.1, 1.7 Hz, 1H), 7.09 (d, half of AB quartet, J=1.6 Hz, 1H), 6.76 (d, J=8.5 Hz, 2H), 3.95 (s, 3H), 3.80 (br s, 2H), 1.60 (s, 9H, assumed; partially obscured by water peak).

### Step 3. Synthesis of tert-butyl 3-methoxy-4′-[(1-{[4-(propan-2-yl)phenyl]carbamoyl}-D-prolyl)amino][1,1′-biphenyl]-4-carboxylate (C29)

To a solution of C1 (31.0 g, 112 mmol) and C28 (33.6 g, 112 mmol) in N,N-dimethylacetamide (250 mL) was added 1-[3-(dimethylamino)propyl]-3-ethylcarbodiimide hydrochloride (25.8 g, 135 mmol) in one portion. After 30 minutes, the reaction mixture was poured into water (1 L) with stirring; the resulting solid was collected via filtration, washed with water, and dissolved in ethyl acetate (700 mL). This solution was washed with saturated aqueous sodium chloride solution, dried over magnesium sulfate, filtered, and concentrated in vacuo. The residue was slurried in heptane and filtered, affording C29 as a pale-tan solid. Yield: 61.4 g, 110 mmol, 98%. LCMS m/z 558.6 [M+H]⁺. ¹H NMR (400 MHz, DMSO-d₆) δ 10.14 (s, 1H), 8.19 (s, 1H), 7.72 (AB quartet, J_{AB}=9.2 Hz, Δv_{AB}=6.6 Hz, 4H), 7.62 (d, J=8.1 Hz, 1H), 7.40 (d, J=8.6 Hz, 2H), 7.31 (d, half of AB quartet, J=1.6 Hz, 1H), 7.26 (dd, component of ABX system, J=8.0, 1.6 Hz, 1H), 7.09 (d, J=8.6 Hz, 2H), 4.48 (dd, J=8.3, 3.6 Hz, 1H), 3.90 (s, 3H), 3.69-3.61 (m, 1H), 3.56-3.47 (m, 1H), 2.80 (septet, J=6.9 Hz, 1H), 2.25-2.13 (m, 1H), 2.10-1.88 (m, 3H), 1.52 (s, 9H), 1.16 (d, J=6.9 Hz, 6H).

### Step 4. Synthesis of 3-methoxy-4′-[(1-{[4-(propan-2-yl)phenyl]carbamoyl}-D-prolyl)amino][1,1′-biphenyl]-4-carboxylic acid (21)

Trifluoroacetic acid (12 mL, 160 mmol) was added to a solution of C29 (12.4 g, 22.2 mmol) in dichloromethane (150 mL). After 30 minutes at room temperature, the reaction mixture was concentrated under reduced pressure. To the residue was added propan-2-yl acetate (100 mL) and the resulting solution was stirred for 20 minutes, whereupon the precipitate was collected via filtration. The filter cake was washed with propan-2-yl acetate (20 mL) to provide 3-methoxy-4′-[(1-{[4-(propan-2-yl)phenyl]carbamoyl}-D-prolyl)amino][1,1′-biphenyl]-4-carboxylic acid (21) as a colorless solid. Yield: 9.84 g, 19.6 mmol, 88%. LCMS m/z 502.4 [M+H]⁺. ¹H NMR (400 MHz, DMSO-d₆) δ 12.51 (v br s, 1H), 10.14 (s, 1H), 8.19 (s, 1H), 7.82-7.65 (m, 5H), 7.40 (d, J=8.1 Hz, 2H), 7.32 (s, 1H), 7.27 (d, J=8.2 Hz, 1H), 7.09 (d, J=8.1 Hz, 2H), 4.52-4.43 (m, 1H), 3.92 (s, 3H), 3.70-3.60 (m, 1H), 3.57-3.46 (m, 1H), 2.86-2.74 (m, 1H), 2.26-2.12 (m, 1H), 2.10-1.86 (m, 3H), 1.16 (d, J=6.9 Hz, 6H).

### Example 22

### 4-{5-[(1-{[4-(Trifluoromethyl)phenyl]carbamoyl}-D-prolyl)amino]pyridin-2-yl}benzoic acid (22)

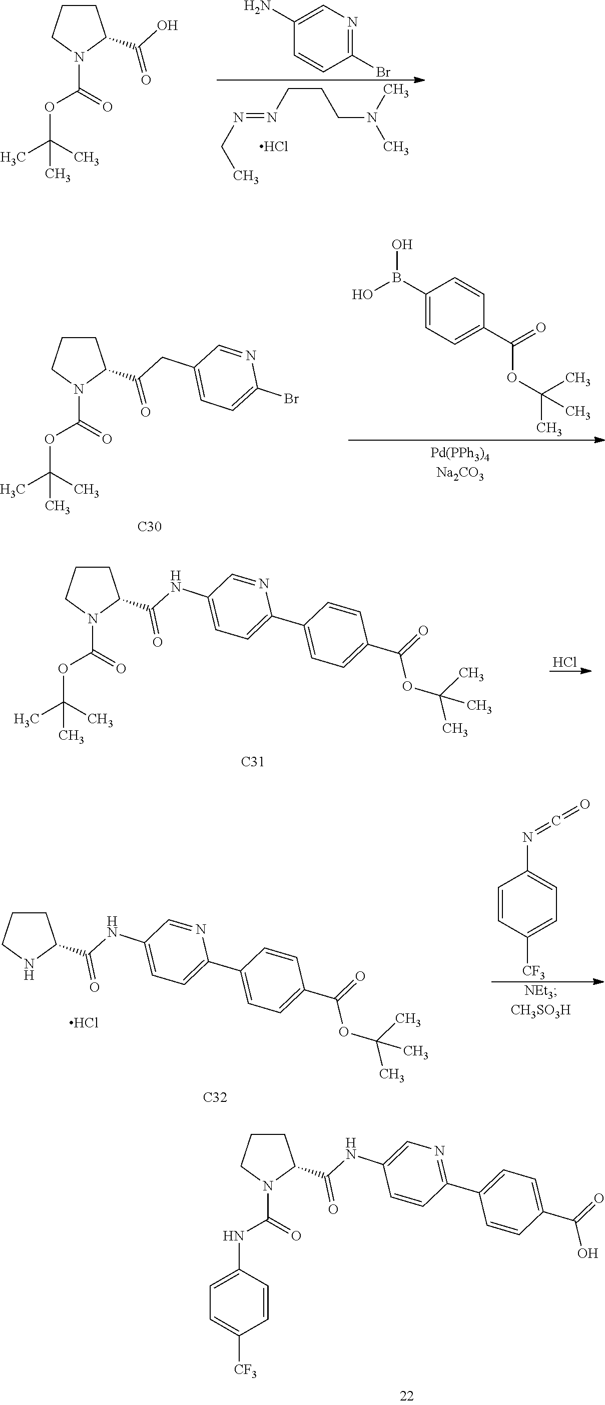

### Step 1. Synthesis of tert-butyl (2R)-2-[(6-bromopyridin-3-yl)carbamoyl]pyrrolidine-1-carboxylate (C30)

A solution of 1-(tert-butoxycarbonyl)-D-proline (2.15 g, 10.0 mmol), 6-bromopyridin-3-amine (1.73 g, 10.0 mmol), and 1-[3-(dimethylamino)propyl]-3-ethylcarbodiimide hydrochloride (2.49 g, 13.0 mmol) in dichloromethane (50 mL) was stirred at room temperature for 3 hours. The reaction mixture was then diluted with dichloromethane (50 mL); the organic layer was washed sequentially with water (2×50 mL) and saturated aqueous sodium chloride solution (40 mL), dried over sodium sulfate, filtered, and concentrated in vacuo. Trituration of the residue with diethyl ether, followed by washing of the filtered solid with diethyl ether (3×25 mL), provided C30 as an off-white solid. Yield: 2.29 g, 6.18 mmol, 62%. LCMS m/z 370.2 [M+H]⁺. ¹H NMR (400 MHz, DMSO-d₆) δ 10.35 (s, 1H), 8.62-8.58 (m, 1H), 8.03-7.96 (m, 1H), 7.64-7.56 (m, 1H), [4.25 (dd, J=8.4, 3.2 Hz) and 4.19 (dd, J=8.3, 4.4 Hz), total 1H], 3.47-3.38 (m, 1H), 3.38-3.3 (m, 1H, assumed; partially obscured by water peak), 2.29-2.11 (m, 1H), 1.95-1.74 (m, 3H), 1.39 (s, 3H), 1.26 (s, 6H).

### Step 2. Synthesis of tert-butyl (2R)-2-({6-[4-(tert-butoxycarbonyl)phenyl]pyridin-3-yl}carbamoyl)pyrrolidine-1-carboxylate (C31)

A vial was charged with C30 (370 mg, 1.00 mmol), [4-(tert-butoxycarbonyl)phenyl]boronic acid (266 mg, 1.20 mmol), sodium carbonate (324 mg, 3.06 mmol), and tetrakis(triphenylphosphine)palladium(0) (86.7 mg, 75.0 μmol), whereupon it was evacuated and charged with nitrogen; this evacuation cycle was repeated twice. 1,2-Dimethoxyethane (4.0 mL) and water (1.5 mL) were added via syringe, and the reaction mixture was heated at 90° C. for 18 hours. LCMS analysis indicated conversion to C31: LCMS m/z 468.4 [M+H]⁺, and the reaction mixture was cooled to room temperature and diluted with ethyl acetate (40 mL). The organic layer was washed sequentially with water (2×20 mL) and saturated aqueous sodium chloride solution (20 mL), dried over sodium sulfate, filtered, and concentrated in vacuo. Silica gel chromatography (Gradient: 50% to 80% ethyl acetate in heptane) afforded C31 as an off-white solid. Yield: 248 mg, 0.530 mmol, 53%. ¹H NMR (400 MHz, DMSO-d₆) δ 10.38 (s, 1H), 8.90-8.84 (m, 1H), 8.23-8.18 (m, 1H), 8.17 (d, J=8.5 Hz, 2H), 8.07-8.01 (m, 1H), 7.98 (d, J=8.5 Hz, 2H), [4.30 (dd, J=8.5, 3.2 Hz) and 4.23 (dd, J=8.2, 4.5 Hz), total 1H], 3.50-3.40 (m, 1H), 3.40-3.32 (m, 1H), 2.31-2.14 (m, 1H), 1.98-1.75 (m, 3H), 1.57 (s, 9H), 1.41 (s, 3H), 1.28 (s, 6H).

### Step 3. Synthesis of tert-butyl 4-[5-(D-prolylamino)pyridin-2-yl]benzoate, hydrochloride salt (C32)

A solution of hydrogen chloride in 1,4-dioxane (4.0 M; 1.33 mL, 5.32 mmol) was added to a solution of C31 (248 mg, 0.530 mmol) in a mixture of 1,4-dioxane (10 mL) and dichloromethane (3.0 mL). After the reaction mixture had been stirred for 3 days at room temperature, it was filtered; the filter cake was washed with methyl tert-butyl ether (2×30 mL) to provide C32 as a white solid. Yield: 196 mg, 0.485 mmol, 92%. LCMS m/z 368.3 [M+H]⁺. ¹H NMR (400 MHz, DMSO-d₆) δ 11.59 (s, 1H), 10.18-10.05 (m, 1H), 9.01 (d, J=2.4 Hz, 1H), 8.83-8.70 (m, 1H), 8.26 (dd, component of ABX system, J=8.7, 2.5 Hz, 1H), 8.18 (d, J=8.5 Hz, 2H), 8.13 (d, half of AB quartet, J=8.7 Hz, 1H), 8.00 (d, J=8.5 Hz, 2H), 4.54-4.43 (m, 1H), 3.36-3.21 (m, 2H), 2.5-2.41 (m, 1H, assumed; partially obscured by solvent peak), 2.09-1.89 (m, 3H), 1.57 (s, 9H).

### Step 4. Synthesis of 4-{5-[(1-{[4-(trifluoromethyl)phenyl]carbamoyl}-D-prolyl)amino]pyridin-2-yl}benzoic acid (22)

1-Isocyanato-4-(trifluoromethyl)benzene (18.7 mg, 0.100 mmol) was added to a solution of C32 (40.4 mg, 0.100 mmol) and triethylamine (16.7 μL, 0.120 mmol) in dichloromethane (0.5 mL). After the reaction mixture had been stirred at room temperature for 1 hour, LCMS analysis indicated the presence of the urea product, the tert-butyl ester of 22: LCMS m/z 555.4 [M+H]⁺. Saturated aqueous sodium chloride solution (1 mL) was added to the reaction mixture, and the aqueous layer was extracted with ethyl acetate (2×2 mL), whereupon the combined organic layers were dried via filtration through a cartridge packed with magnesium sulfate. Solvent was removed using a Genevac concentrator; the residue was dissolved in 1,1,1,3,3,3-hexafluoropropan-2-ol (0.5 mL) and then treated with methanesulfonic acid (9.6 mg, 0.100 mmol). After this reaction mixture had been stirred for 15 minutes at room temperature, it was concentrated in vacuo and purified using reversed-phase HPLC (Column: Waters Sunfire C18, 19×100 mm, 5 μm; Mobile phase A: 0.05% trifluoroacetic acid in water (v/v); Mobile phase B: 0.05% trifluoroacetic acid in acetonitrile (v/v); Gradient: 20% to 60% B over 8.5 minutes, then 60% to 95% B over 0.5 minutes, then 95% B for 1.0 minute; Flow rate: 25 mL/minute), providing 4-{5-[(1-{[4-(trifluoromethyl)phenyl]carbamoyl}-D-prolyl)amino]pyridin-2-yl}benzoic acid (22). Yield: 24.9 mg, 50.0 μmol, 50%. LCMS m/z 499.3 [M+H]⁺. ¹H NMR (600 MHz, DMSO-d₆) δ 10.42 (s, 1H), 8.88 (d, J=2.6 Hz, 1H), 8.72 (s, 1H), 8.20 (dd, J=8.7, 2.5 Hz, 1H), 8.16 (d, J=8.3 Hz, 2H), 8.05-8.00 (m, 3H), 7.75 (d, J=8.5 Hz, 2H), 7.58 (d, J=8.6 Hz, 2H), 4.51 (dd, J=8.3, 3.9 Hz, 1H), 3.72-3.65 (m, 1H), 3.61-3.54 (m, 1H), 2.29-2.19 (m, 1H), 2.09-1.93 (m, 3H).

### Alternate Synthesis of Example 22

### 4-{5-[(1-{[4-(Trifluoromethyl)phenyl]carbamoyl}-D-prolyl)amino]pyridin-2-yl}benzoic acid (22)

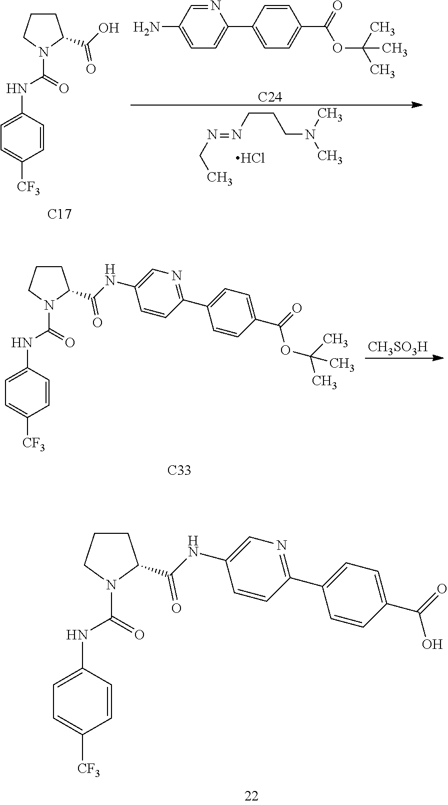

### Step 1. Synthesis of tert-butyl 4-{5-[(1-{[4-(trifluoromethyl)phenyl]carbamoyl}-D-prolyl)amino]pyridin-2-yl}benzoate (C33)

A mixture of C17 (350 mg, 1.16 mmol), C24 (313 mg, 1.16 mmol), and 1-[3-(dimethylamino)propyl]-3-ethylcarbodiimide hydrochloride (266 mg, 1.39 mmol) in dichloromethane (5.8 mL) was stirred at 25° C. for 2.5 hours. Filtration afforded C33 as a fluffy white solid, which was used in the following step without further purification. Yield: 365 mg, 0.658 mmol, 57%. LCMS m/z 555.5 [M+H]⁺.

### Step 2. Synthesis of 4-{5-[(1-{[4-(trifluoromethyl)phenyl]carbamoyl}-D-prolyl)amino]pyridin-2-yl}benzoic acid (22)

Methanesulfonic acid (0.513 mL, 7.91 mmol) was added to a solution of C33 (365 mg, 0.658 mmol) in acetonitrile (3.3 mL). After 3 hours at 25° C., LCMS analysis indicated the presence of 22: LCMS m/z 499.4 [M+H]⁺. The acetonitrile was removed via concentration under reduced pressure, and the residue was diluted with water (5 mL). The resulting mixture was adjusted to pH 8 by addition of saturated sodium bicarbonate solution, whereupon 1 M hydrochloric acid was added until a precipitate formed, near pH 6. This mixture was allowed to stir at room temperature overnight and was then brought to pH 3 by the further addition of 1 M hydrochloric acid. The resulting solid was isolated by filtration to afford 4-{5-[(1-{[4-(trifluoromethyl)phenyl]carbamoyl}-D-prolyl)amino]pyridin-2-yl}benzoic acid (22) as a white solid. Yield: 194 mg, 0.389 mmol, 59%. ¹H NMR (400 MHz, DMSO-d₆) δ 12.98 (br s, 1H), 10.41 (s, 1H), 8.88 (d, J=2.5 Hz, 1H), 8.72 (s, 1H), 8.20 (dd, J=8.8, 2.6 Hz, 1H), 8.17 (d, J=8.5 Hz, 2H), 8.04 (d, J=8.1 Hz, 1H), 8.02 (d, J=8.5 Hz, 2H), 7.76 (d, J=8.5 Hz, 2H), 7.58 (d, J=8.6 Hz, 2H), 4.52 (dd, J=8.5, 3.6 Hz, 1H), 3.74-3.64 (m, 1H), 3.63-3.54 (m, 1H), 2.28-2.19 (m, 1H), 2.11-1.92 (m, 3H).

### Example 23

### 4′-({1-[(4-Cyclobutylphenyl)carbamoyl]-D-prolyl}amino)[1,1′-biphenyl]-4-carboxylic acid (23)

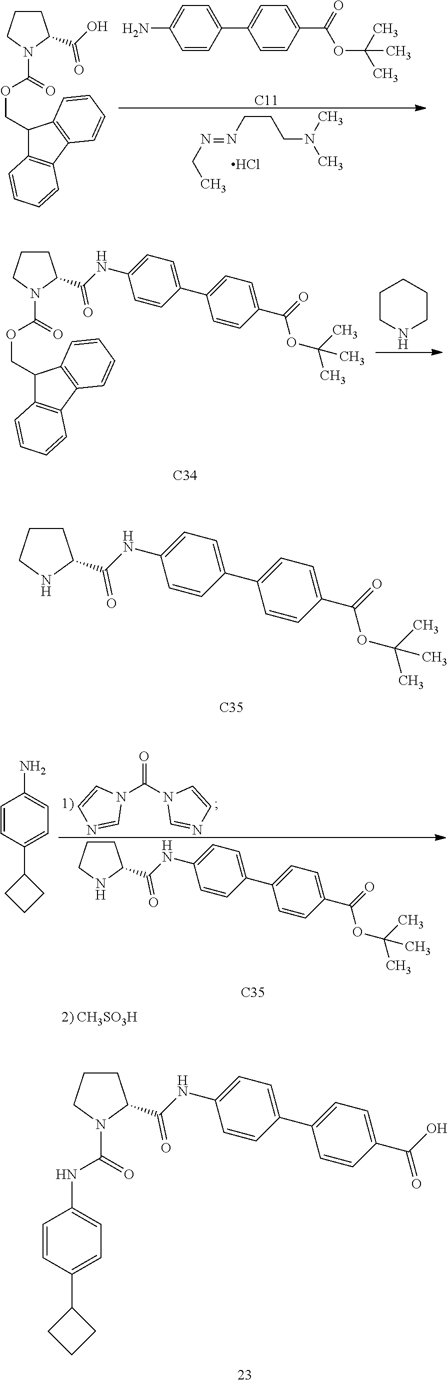

### Step 1. Synthesis of (9H-fluoren-9-yl)methyl (2R)-2-{[4′-(tert-butoxycarbonyl)[1,1′-biphenyl]-4-yl]carbamoyl}pyrrolidine-1-carboxylate (C34)

A mixture of 1-{[(9H-fluoren-9-yl)methoxy]carbonyl}-D-proline (7.52 g, 22.3 mmol), C11 (6.00 g, 22.3 mmol), and 1-[3-(dimethylamino)propyl]-3-ethylcarbodiimide hydrochloride (5.12 g, 26.7 mmol) in dichloromethane (200 mL) was stirred at room temperature for 16 hours, whereupon the reaction mixture was poured into water and extracted with dichloromethane. The organic layer was dried over magnesium sulfate, filtered, and concentrated in vacuo to provide C34 as a gray solid, which was progressed directly to the following step. LCMS m/z 589.4 [M+H]⁺.

### Step 2. Synthesis of tert-butyl 4′-(D-prolylamino)[1,1′-biphenyl]-4-carboxylate (C35)

Piperidine (10 mL, 110 mmol) was added to a solution of C34 (from the previous step; ≤22.3 mmol) in N,N-dimethylformamide (100 mL). After the reaction mixture had been stirred at 25° C. for 1 hour, it was partitioned between ethyl acetate and water. The organic layer was washed with saturated aqueous sodium chloride solution, dried over sodium sulfate, filtered, concentrated in vacuo, and purified via silica gel chromatography (Gradient: 0% to 10% methanol in dichloromethane) to afford C35 as a gray solid. Yield: 7.20 g, 19.6 mmol, 88% over 2 steps. LCMS m/z 367.3 [M+H]⁺. ¹H NMR (400 MHz, chloroform-d) δ 10.06 (s, 1H), 8.03 (d, J=8.5 Hz, 2H), 7.73 (d, J=8.7 Hz, 1H), 7.63-7.56 (m, 4H), 4.13 (dd, J=9.0, 5.5 Hz, 1H), 3.20 (dt, J=10.5, 6.8 Hz, 1H), 3.10 (dt, J=10.4, 6.4 Hz, 1H), 2.40-2.27 (m, 1H), 2.15-2.03 (m, 1H), 1.90-1.79 (m, 2H), 1.61 (s, 9H).

### Step 3. Synthesis of 4′-({1-[(4-cyclobutylphenyl)carbamoyl]-D-prolyl}amino)[1,1′-biphenyl]-4-carboxylic acid (23)

A solution of 4-cyclobutylaniline (100 mg, 0.680 mmol) and 1,1′-carbonyldiimidazole (111 mg, 0.685 mmol) in dichloromethane (5.0 mL) was stirred for 30 minutes at room temperature, whereupon C35 (250 mg, 0.682 mmol) was added. After the reaction mixture had been stirred for 1 hour at room temperature, saturated aqueous sodium chloride solution (15 mL) was added. The aqueous layer was extracted with ethyl acetate (2×50 mL), and the combined organic layers were concentrated in vacuo; the resulting solid was stirred in dichloromethane for 5 minutes, isolated via filtration, and taken up in 1,1,1,3,3,3-hexafluoropropan-2-ol (2 mL). This solution was treated with methanesulfonic acid (44.3 μL, 0.683 mmol) and stirred for 3 hours at room temperature. Upon addition of dichloromethane (2 mL), a solid precipitated; the mixture was stirred for 1 hour before collection of the solid by filtration. This material was stirred in a mixture of tetrahydrofuran and acetonitrile (10:1, 2 mL) for 1 hour, whereupon it was filtered to provide 4′-({1-[(4-cyclobutylphenyl)carbamoyl]-D-prolyl}amino)[1,1′-biphenyl]-4-carboxylic acid (23) as a white solid. Yield: 205 mg, 0.424 mmol, 62%. LCMS m/z 484.4 [M+H]⁺. ¹H NMR (400 MHz, DMSO-d₆) δ 12.91 (br s, 1H), 10.14 (s, 1H), 8.21 (s, 1H), 7.99 (d, J=8.5 Hz, 2H), 7.78 (d, J=8.5 Hz, 2H), 7.72 (AB quartet, J_{AB}=9.0 Hz, Δv_{AB}=12.5 Hz, 4H), 7.42 (d, J=8.6 Hz, 2H), 7.08 (d, J=8.5 Hz, 2H), 4.47 (dd, J=8.3, 3.6 Hz, 1H), 3.69-3.61 (m, 1H), 3.56-3.47 (m, 1H), 3.42 (pentet, J=8.6 Hz, 1H), 2.29-2.14 (m, 3H), 2.10-1.87 (m, 6H), 1.83-1.72 (m, 1H).

### Example 24

### 4-{5-Fluoro-6-[(1-{[4-(trifluoromethyl)phenyl]carbamoyl}-D-prolyl)amino]pyridin-3-yl}benzoic acid (24)

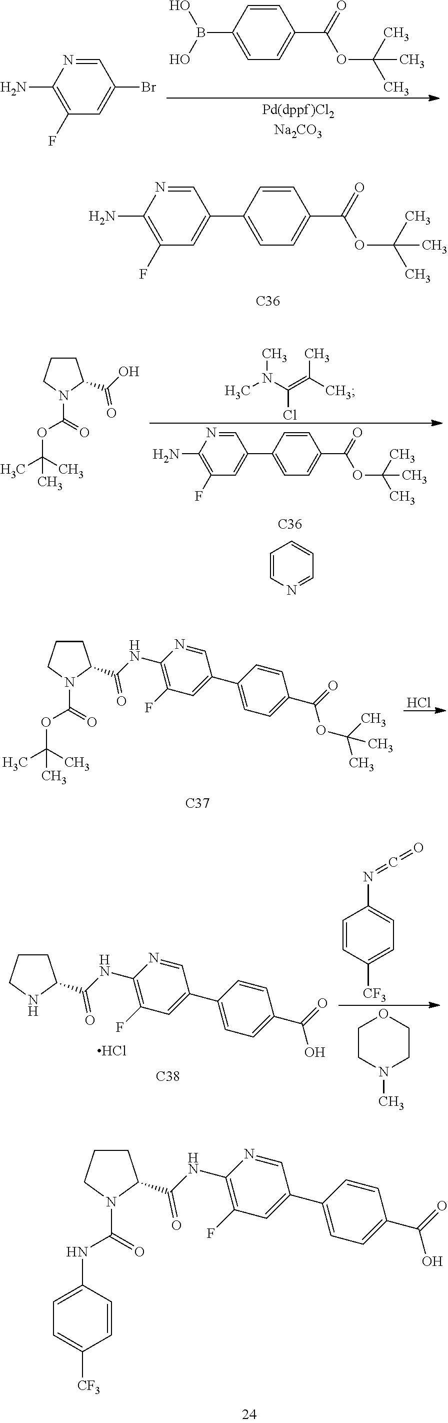

### Step 1. Synthesis of tert-butyl 4-(6-amino-5-fluoropyridin-3-yl)benzoate (C36)

A mixture of 5-bromo-3-fluoropyridin-2-amine (500 mg, 2.62 mmol), [4-(tert-butoxycarbonyl)phenyl]boronic acid (639 mg, 2.88 mmol), sodium carbonate (832 mg, 7.85 mmol), and [1,1′-bis(diphenylphosphino)ferrocene]dichloropalladium(II) (192 mg, 0.262 mmol) in a mixture of 1,4-dioxane (10 mL) and water (2 mL) was heated at 80° C. for 1.5 hours, then allowed to stand overnight. After the 1,4-dioxane had been removed in vacuo, the residue was partitioned between ethyl acetate and water, and the resulting mixture was filtered through diatomaceous earth. The organic layer of the filtrate was washed with saturated aqueous sodium chloride solution, dried over magnesium sulfate, filtered, and concentrated in vacuo; silica gel chromatography (Gradient: 20% to 50% ethyl acetate in heptane) provided C36 as a tan solid. Yield: 563 mg, 1.95 mmol, 74%. LCMS m/z 289.2 [M+H]⁺. ¹H NMR (400 MHz, DMSO-d₆) δ 8.24-8.21 (m, 1H), 7.91 (d, J=8.5 Hz, 2H), 7.81 (dd, J=12.7, 2.0 Hz, 1H), 7.76 (d, J=8.5 Hz, 2H), 6.52 (s, 2H), 1.56 (s, 9H).

### Step 2. Synthesis of tert-butyl (2R)-2-({5-[4-(tert-butoxycarbonyl)phenyl]-3-fluoropyridin-2-yl}carbamoyl)pyrrolidine-1-carboxylate (C37)

1-Chloro-N,N,2-trimethylprop-1-en-1-amine (Ghosez's reagent; 92 μL, 0.695 mmol) was added to a solution of 1-(tert-butoxycarbonyl)-D-proline (150 mg, 0.697 mmol) in dichloromethane (5 mL). After the reaction mixture had been stirred at room temperature for 30 minutes, C36 (201 mg, 0.697 mmol) was added, followed by pyridine (0.172 mL, 2.13 mmol), and stirring was continued overnight. The reaction mixture was partitioned between dichloromethane and water, and the organic layer was washed with saturated aqueous sodium chloride solution, dried over magnesium sulfate, filtered, and concentrated in vacuo. Silica gel chromatography (Gradient: 30% to 50% ethyl acetate in heptane) afforded C37 as a colorless foam; ¹H NMR analysis indicated that this material exists as a mixture of rotamers. Yield: 235 mg, 0.484 mmol, 69%. LCMS m/z 486.4 [M+H]⁺. ¹H NMR (400 MHz, DMSO-d₆) δ [10.45 (s) and 10.43 (s), total 1H], 8.69-8.64 (m, 1H), [8.22-8.16 (m) and 8.20 (dd, J=11.2, 2.0 Hz), total 1H], 7.96 (AB quartet, J_{AB}=8.5 Hz, Δv_{AB}=25.7 Hz, 4H), [4.46-4.39 (m) and 4.35 (dd, J=8.5, 4.0 Hz), total 1H], 3.49-3.39 (m, 1H), 3.39-3.3 (m, 1H, assumed; partially obscured by water peak), 2.32-2.14 (m, 1H), 1.98-1.74 (m, 3H), 1.57 (s, 9H), [1.40 (s) and 1.36 (s), total 9H].

### Step 3. Synthesis of 4-[5-fluoro-6-(D-prolylamino)pyridin-3-yl]benzoic acid, hydrochloride salt (C38)

A solution of hydrogen chloride in 1,4-dioxane (4.0 M; 4.0 mL, 16 mmol) was added to C37 (235 mg, 0.484 mmol) and the reaction mixture was allowed to stir at room temperature for 1.5 hours, whereupon it was diluted with diethyl ether (50 mL). Solids were collected via filtration and washed with diethyl ether to provide C38 as a colorless solid. Yield: 150 mg, 0.410 mmol, 85%. LCMS m/z 330.2 [M+H]⁺. ¹H NMR (400 MHz, DMSO-d₆) δ 11.16 (s, 1H), 10.01-9.88 (m, 1H), 8.79-8.68 (m, 2H), 8.29 (dd, J=11.2, 2.0 Hz, 1H), 8.00 (AB quartet, J_{AB}=8.5 Hz, Δv_{AB}=40.5 Hz, 4H), 4.58-4.45 (m, 1H), 3.34-3.21 (m, 2H), 2.5-2.41 (m, 1H, assumed; partially obscured by solvent peak), 2.06-1.88 (m, 3H).

### Step 4. Synthesis of 4-{5-fluoro-6-[(1-{[4-(trifluoromethyl)phenyl]carbamoyl}-D-prolyl)amino]pyridin-3-yl}benzoic acid (24)

To a solution of C38 (35 mg, 96 μmol) in N,N-dimethylacetamide (1 mL) was added 4-methylmorpholine (11 μL, 0.10 mmol); after the mixture had been stirred for 2 minutes, 1-isocyanato-4-(trifluoromethyl)benzene (18 mg, 96 μmol) was added, and the reaction mixture was stirred at room temperature for 15 minutes. It was then diluted with water (10 mL) and filtered. The resulting solid was washed with water to afford 4-{5-fluoro-6-[(1-{[4-(trifluoromethyl)phenyl]carbamoyl}-D-prolyl)amino]pyridin-3-yl}benzoic acid (24) as a colorless solid. Yield: 27 mg, 52 μmol, 54%. LCMS m/z 517.2 [M+H]⁺. ¹H NMR (400 MHz, DMSO-d₆) δ 13.08 (br s, 1H), 10.46 (s, 1H), 8.69 (s, 1H), 8.68-8.66 (m, 1H), 8.19 (dd, J=11.2, 2.0 Hz, 1H), 7.98 (AB quartet, J_{AB}=8.5 Hz, Δv_{AB}=45.1 Hz, 4H), 7.77 (d, J=8.6 Hz, 2H), 7.59 (d, J=8.7 Hz, 2H), 4.67-4.59 (m, 1H), 3.75-3.65 (m, 1H), 3.60-3.50 (m, 1H), 2.31-2.19 (m, 1H), 2.07-1.93 (m, 3H).

### Example 25

### 4-{5-Fluoro-6-[(1-{[4-(propan-2-yl)phenyl]carbamoyl}-D-prolyl)amino]pyridin-3-yl}benzoic acid (25)

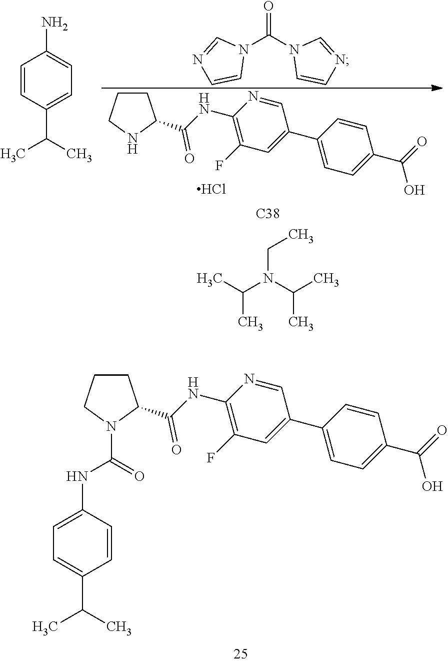

To a 0° C. solution of 4-(propan-2-yl)aniline (31.0 mg, 0.229 mmol) in acetonitrile (4.0 mL) was added 1,1′-carbonyldiimidazole (37.0 mg, 0.228 mmol). After the reaction mixture had been stirred at 0° C. for 20 minutes, C38 (75 mg, 0.21 mmol) was added, followed by N,N-diisopropylethylamine (40 μL, 30 mg, 0.23 mmol). The cooling bath was then removed, and the reaction mixture was allowed to warm to room temperature; after 1 hour, it was concentrated in vacuo and purified via reversed-phase HPLC (Column: Waters Sunfire C18, 19×100 mm, 5 μm; Mobile phase A: 0.05% trifluoroacetic acid in water (v/v); Mobile phase B: 0.05% trifluoroacetic acid in acetonitrile (v/v); Gradient: 20% to 60% B over 8.5 minutes, then 60% to 95% B over 0.5 minutes, then 95% B for 1.0 minute; Flow rate: 25 mL/minute) to afford 4-{5-fluoro-6-[(1-{[4-(propan-2-yl)phenyl]carbamoyl}-D-prolyl)amino]pyridin-3-yl}benzoic acid (25). Yield: 22.8 mg, 46.5 μmol, 22%. LCMS m/z 491.4 [M+H]⁺. ¹H NMR (600 MHz, DMSO-d₆), characteristic peaks: δ 10.42 (s, 1H), 8.66 (s, 1H), 8.21-8.16 (m, 2H), 8.03 (d, J=8.4 Hz, 2H), 7.92 (br d, J=8.3 Hz, 2H), 7.43-7.38 (m, 2H), 7.10 (d, J=8.4 Hz, 2H), 4.63-4.56 (m, 1H), 3.69-3.62 (m, 1H, assumed; partially obscured by water peak), 2.81 (septet, J=6.8 Hz, 1H), 2.25-2.17 (m, 1H), 2.05-1.94 (m, 3H), 1.16 (d, J=6.8 Hz, 6H).

### Example 26

### 4′-[(1-{[4-Cyclopropyl-3-(trifluoromethyl)phenyl]carbamoyl}-D-prolyl)amino][1,1′-biphenyl]-4-carboxylic acid (26)

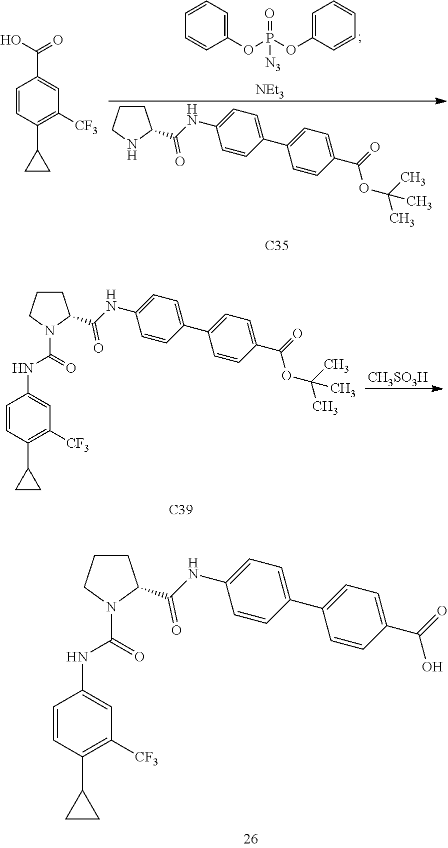

### Step 1. Synthesis of tert-butyl 4′-[(1-{[4-cyclopropyl-3-(trifluoromethyl)phenyl]carbamoyl}-D-prolyl)amino][1,1′-biphenyl]-4-carboxylate (C39)

Diphenyl phosphorazidate (21.4 μL, 99.3 μmol) was added to a solution of 4-cyclopropyl-3-(trifluoromethyl)benzoic acid (20 mg, 87 μmol) and triethylamine (12.7 μL, 91.1 μmol) in toluene (1 mL), whereupon the reaction mixture was gradually heated to 110° C. After stirring at 110° C. for one hour, it was cooled to 40° C., and C35 (31.8 mg, 86.8 μmol) was added. Stirring was continued at 40° C. for 1.5 hours, at which point the reaction mixture was allowed to cool to room temperature, stirred over the weekend, and concentrated in vacuo. Silica gel chromatography (Gradient: 0% to 100% ethyl acetate in heptane) afforded C39 as a solid. Yield: 31.0 mg, 52.2 μmol, 60%. LCMS m/z 594.5 [M+H]⁺.

### Step 2. Synthesis of 4′-[(1-{[4-cyclopropyl-3-(trifluoromethyl)phenyl]carbamoyl}-D-prolyl)amino][1,1′-biphenyl]-4-carboxylic acid (26)

Methanesulfonic acid (3.73 μL, 57.5 μmol) was added to a solution of C39 (31.0 mg, 52.2 μmol) in 1,1,1,3,3,3-hexafluoropropan-2-ol (0.5 mL). After 2 hours, the reaction mixture was concentrated under a stream of nitrogen; the residue was purified via reversed-phase HPLC (Column: Waters Sunfire C18, 19×100 mm, 5 μm; Mobile phase A: 0.05% trifluoroacetic acid in water (v/v); Mobile phase B: 0.05% trifluoroacetic acid in acetonitrile (v/v); Gradient: 5% to 95% B over 8.54 minutes, followed by 95% B for 1.46 minutes; Flow rate: 25 mL/minute) to afford 4′-[(1-{[4-cyclopropyl-3-(trifluoromethyl)phenyl]carbamoyl}-D-prolyl)amino][1,1′-biphenyl]-4-carboxylic acid (26). Yield: 16.3 mg, 30.3 μmol, 58%. LCMS m/z 538.4 [M+H]⁺. ¹H NMR (600 MHz, DMSO-d₆) δ 10.15 (s, 1H), 8.54 (s, 1H), 7.99 (d, J=8.4 Hz, 2H), 7.95-7.92 (m, 1H), 7.78 (d, J=8.4 Hz, 2H), 7.72 (AB quartet, J_{AB}=8.8 Hz, Δv_{AB}=15.2 Hz, 4H), 7.67 (br d, J=8.7 Hz, 1H), 7.04 (d, J=8.7 Hz, 1H), 4.47 (dd, J=8.4, 3.8 Hz, 1H), 3.68-3.62 (m, 1H), 3.56-3.50 (m, 1H, assumed; partially obscured by water peak), 2.25-2.16 (m, 1H), 2.08-1.90 (m, 4H), 0.97-0.91 (m, 2H), 0.73-0.68 (m, 2H).

### Example 27

### 5-{4-[(1-{[3-Fluoro-4-(propan-2-yl)phenyl]carbamoyl}-D-prolyl)amino]phenyl}-6-methylpyridine-2-carboxylic acid (27)

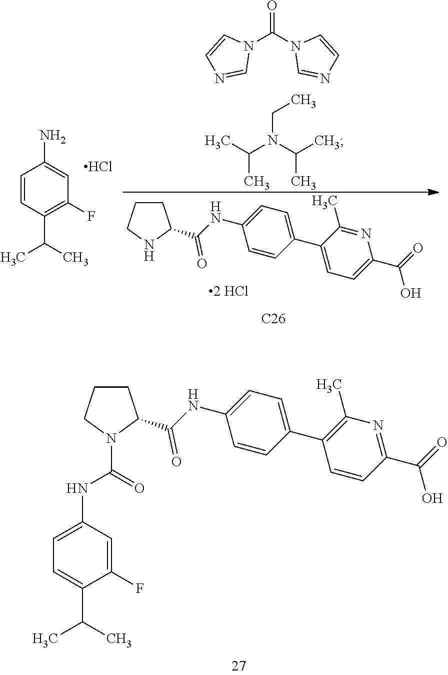

1,1′-Carbonyldiimidazole (358 mg, 2.21 mmol) was added to a solution of 3-fluoro-4-(propan-2-yl)aniline, hydrochloride salt (400 mg, 2.11 mmol) and N,N-diisopropylethylamine (0.367 mL, 2.11 mmol) in acetonitrile (10 mL). After the reaction mixture had been stirred at 22° C. for 30 minutes, it was added to a 0° C. solution of C26 (800 mg, 2.01 mmol) and N,N-diisopropylethylamine (700 μL, 4.02 mmol) in acetonitrile (5 mL). The reaction mixture was allowed to stir at room temperature for 18 hours, whereupon it was diluted with water (5 mL) and the pH was adjusted to 4 to 5 by addition of 1 M hydrochloric acid. The aqueous layer was extracted with ethyl acetate (2×100 mL), and the combined organic layers were concentrated in vacuo; silica gel chromatography (Gradient: 0% to 10% methanol in dichloromethane) provided a solid, which was stirred in methyl tert-butyl ether for three days, isolated via filtration, and washed with methyl tert-butyl ether, affording 5-{4-[(1-{[3-fluoro-4-(propan-2-yl)phenyl]carbamoyl}-D-prolyl)amino]phenyl}-6-methylpyridine-2-carboxylic acid (27) as a white solid. Yield: 360 mg, 0.713 mmol, 35%. LCMS m/z 503.4 [M−H]⁻. ¹H NMR (400 MHz, DMSO-d₆) δ 10.16 (s, 1H), 8.40 (s, 1H), 7.92 (d, J=7.9 Hz, 1H), 7.75 (d, J=7.8 Hz, 1H), 7.74 (d, J=8.5 Hz, 2H), 7.45-7.35 (m, 3H), 7.24 (dd, component of ABX system, J=8.5, 2.1 Hz, 1H), 7.15 (t, J=8.6 Hz, 1H), 4.48 (dd, J=8.4, 3.7 Hz, 1H), 3.70-3.59 (m, 1H), 3.57-3.46 (m, 1H), 3.12-2.99 (m, 1H, assumed; partially obscured by water peak), 2.50 (s, 3H, assumed; coincident with solvent peak), 2.27-2.13 (m, 1H), 2.11-1.87 (m, 3H), 1.17 (d, J=6.9 Hz, 6H).

### Alternate Synthesis of Example 27

### 5-{4-[(1-{[3-Fluoro-4-(propan-2-yl)phenyl]carbamoyl}-D-prolyl)amino]phenyl}-6-methylpyridine-2-carboxylic acid (27)

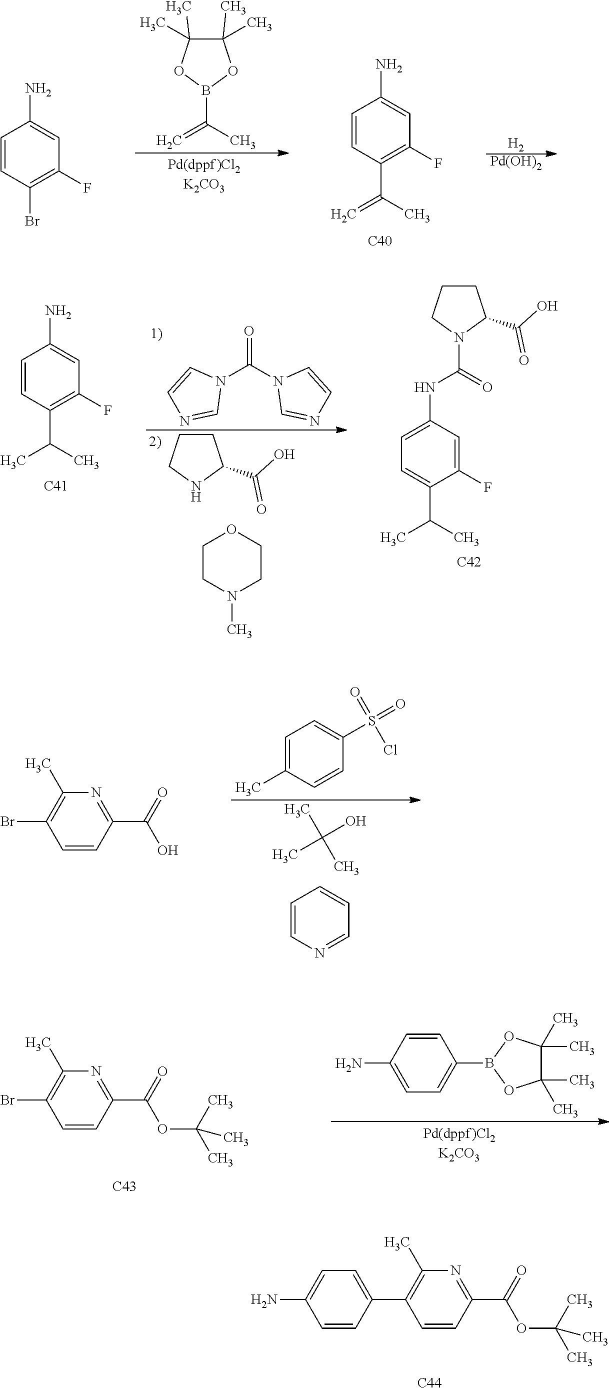

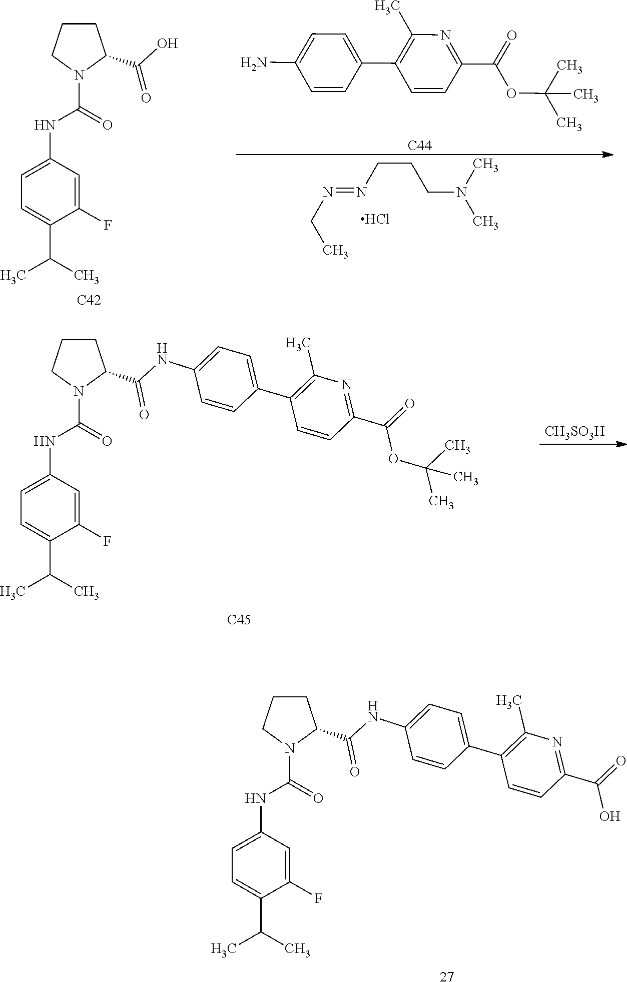

### Step 1. Synthesis of 3-fluoro-4-(prop-1-en-2-yl)aniline (C40)

To a solution of 4-bromo-3-fluoroaniline (5.25 g, 27.6 mmol) in a mixture of 1,4-dioxane (110 mL) and water (11 mL) were added 4,4,5,5-tetramethyl-2-(prop-1-en-2-yl)-1,3,2-dioxaborolane (4.64 g, 27.6 mmol), potassium carbonate (11.5 g, 83.2 mmol), and [1,1′-bis(diphenylphosphino)ferrocene]dichloropalladium(II) (0.607 g, 0.830 mmol). The reaction mixture was heated at 90° C. for 18 hours, whereupon it was diluted with water (100 mL) and extracted with ethyl acetate (3×50 mL). The combined organic layers were washed with saturated aqueous sodium chloride solution (3×60 mL), dried over sodium sulfate, filtered, and concentrated under reduced pressure. The residue was dissolved in diethyl ether (100 mL), treated with hydrochloric acid (1 M; 50 mL) and water (50 mL), and stirred at room temperature for 20 minutes. After the organic layer had been extracted with water (50 mL), the combined aqueous layers were basified to pH 9 by addition of sodium bicarbonate. The resulting mixture was extracted with ethyl acetate (3×60 mL), and the combined organic layers were dried over sodium sulfate, filtered, and concentrated in vacuo, providing an oil (5.0 g). A portion of this material (2.0 g) was purified via chromatography on silica gel (Gradient: 0% to 50% ethyl acetate in heptane) to afford C40 as a yellow oil (1.31 g). Adjusted yield: 0.940 g when corrected for residual ethyl acetate, 6.22 mmol. Adjusting for only 40% of the crude product being purified, reaction yield: 56%. GCMS: m/z 151.1 [M⁺]. ¹H NMR (400 MHz, chloroform-d) δ 7.10 (t, J=8.5 Hz, 1H), 6.40 (dd, component of ABX system, J=8.2, 2.4 Hz, 1H), 6.36 (dd, component of ABX system, J=12.9, 2.3 Hz, 1H), 5.19-5.15 (m, 1H), 5.12-5.08 (m, 1H), 2.12-2.07 (m, 3H).

### Step 2. Synthesis of 3-fluoro-4-(propan-2-yl)aniline (C41)

To a solution of C40 (21.0 g, 139 mmol) in methanol (150 mL) was added palladium hydroxide (20%, 10 g), and the mixture was hydrogenated at 50° C. and 50 psi until GCMS analysis indicated complete consumption of starting material. The reaction mixture was concentrated in vacuo, whereupon it was purified using silica gel chromatography (Gradient: 0% to 50% dichloromethane in hexanes) to afford C41 as a light-colored oil. Yield: 11.4 g, 74.4 mmol, 54%. GCMS m/z 153.1 [M⁺]. ¹H NMR (400 MHz, chloroform-d) δ 7.00 (t, J=8.4 Hz, 1H), 6.42 (dd, component of ABX system, J=8.2, 2.4 Hz, 1H), 6.35 (dd, component of ABX system, J=12.1, 2.4 Hz, 1H), 3.95-3.28 (br m, 2H), 3.11 (septet, J=6.9 Hz, 1H), 1.21 (d, J=6.9 Hz, 6H).

### Step 3. Synthesis of 1-{[3-fluoro-4-(propan-2-yl)phenyl]carbamoyl}-D-proline (C42)

To a 0° C. solution of 1,1′-carbonyldiimidazole (13.2 g, 81.4 mmol) in acetonitrile (170 mL) was added C41 (11.4 g, 74.4 mmol). After the reaction mixture had been stirred at room temperature for 1 hour, it was concentrated under reduced pressure and the residue was dissolved in tetrahydrofuran (5 mL).

To a solution of D-proline (10.3 g, 89.5 mmol) in tetrahydrofuran (10 mL) were added 4-methylmorpholine (9.80 mL, 89.1 mmol) and a solution of the crude intermediate from above in tetrahydrofuran (5 mL), whereupon the reaction mixture was stirred for 2 hours at room temperature. Saturated aqueous sodium bicarbonate solution (5 mL) was added, providing an internal pH of 7 to 8. The resulting mixture was washed with diethyl ether (2×30 mL), acidified to pH 3 with 4 M hydrochloric acid, and extracted with ethyl acetate (3×50 mL); the combined organic layers were washed with saturated aqueous sodium chloride solution (30 mL), dried over sodium sulfate, filtered, and concentrated under reduced pressure to a volume of approximately 30 mL. The resulting heterogeneous mixture was stirred at room temperature for 20 minutes, diluted with methyl tert-butyl ether (125 mL), and stirred for an additional 20 minutes, whereupon heptane (60 mL) was added. After 2 further hours of stirring, solids were collected via filtration and purified using chromatography on silica gel (Gradient: 0% to 5% methanol in dichloromethane) to afford C42 as a white solid (8.71 g). Mixed fractions were subjected to silica gel chromatography (Gradient: 50% to 100% ethyl acetate in heptane) to provide additional C42 as a white solid (3.96 g). Combined yield: 12.7 g, 43.1 mmol, 58%. LCMS m/z 295.3 [M+H]⁺. ¹H NMR (400 MHz, methanol-d₄) δ 7.23 (dd, J=13.0, 2.1 Hz, 1H), 7.15 (dd, component of ABX system, J=8.3, 9.1 Hz, 1H), 7.11 (dd, component of ABX system, J=8.5, 2.0 Hz, 1H), 4.47 (dd, J=8.2, 3.0 Hz, 1H), 3.69-3.59 (m, 1H), 3.58-3.49 (m, 1H), 3.14 (septet, J=7.0 Hz, 1H), 2.34-2.21 (m, 1H), 2.13-2.00 (m, 3H), 1.23 (d, J=6.9 Hz, 6H).

### Step 4. Synthesis of tert-butyl 5-bromo-6-methylpyridine-2-carboxylate (C43)

To a solution of 5-bromo-6-methylpyridine-2-carboxylic acid (20.0 g, 92.6 mmol) in 2-methylpropan-2-ol (450 mL) were added pyridine (35 mL) and 4-methylbenzene-1-sulfonyl chloride (52.9 g, 277 mmol). The reaction mixture was stirred at room temperature overnight, whereupon water (300 mL) was added, followed by solid sodium hydroxide (1.5 g, 38 mmol), providing a pH of 12. The resulting mixture was extracted with ethyl acetate (2×300 mL); the combined organic layers were washed with saturated aqueous sodium chloride solution (3×200 mL), dried over sodium sulfate, filtered, and concentrated in vacuo to afford C43 as a yellow oil. Yield: 24.8 g, 91.1 mmol, 98%. LCMS m/z 272.2 (bromine isotope pattern observed) [M+H]⁺. ¹H NMR (400 MHz, DMSO-d₆) δ 8.14 (d, J=8.2 Hz, 1H), 7.70 (d, J=8.2 Hz, 1H), 2.60 (s, 3H), 1.53 (s, 9H).

### Step 5. Synthesis of tert-butyl 5-(4-aminophenyl)-6-methylpyridine-2-carboxylate (C44)

To a solution of 4-(4,4,5,5-tetramethyl-1,3,2-dioxaborolan-2-yl)aniline (74.9 g, 342 mmol) in a mixture of 1,4-dioxane (1.5 L) and water (300 mL) were added C43 (93.0 g, 342 mmol), potassium carbonate (94.5 g, 684 mmol), and [1,1′-bis(diphenylphosphino)ferrocene]dichloropalladium(II) (12.5 g, 17.1 mmol). After the reaction mixture had been heated at 90° C. for 2 hours, it was combined with a similar reaction carried out using C43 (1.00 g, 3.67 mmol) and concentrated in vacuo. The residue was filtered, whereupon it was extracted with ethyl acetate (2×700 mL); the combined organic layers were washed sequentially with water (300 mL) and saturated aqueous sodium chloride solution (2×600 mL), dried over sodium sulfate, filtered, and concentrated under reduced pressure. Chromatography on silica gel (Gradient: 0% to 30% ethyl acetate in petroleum ether) provided a solid, which was stirred with petroleum ether (60 mL) for 10 minutes. Collection of the solid via filtration, followed by rinsing of the filter cake with petroleum ether (2×6 mL), afforded C44 as a light-yellow solid. Yield: 77.0 g, 271 mmol, 79%. LCMS m/z 285.3 [M+H]⁺. ¹H NMR (400 MHz, DMSO-d₆) δ 7.81 (d, J=7.9 Hz, 1H), 7.66 (d, J=7.9 Hz, 1H), 7.10 (d, J=8.4 Hz, 2H), 6.66 (d, J=8.5 Hz, 2H), 5.34 (s, 2H), 2.50 (s, 3H; assumed; coincident with solvent peak), 1.56 (s, 9H).

### Step 6. Synthesis of tert-butyl 5-{4-[(1-{[3-fluoro-4-(propan-2-yl)phenyl]carbamoyl}-D-prolyl)amino]phenyl}-6-methylpyridine-2-carboxylate (C45)

To a solution of C42 (22.5 g, 76.4 mmol) in dichloromethane (300 mL) was added 1-[3-(dimethylamino)propyl]-3-ethylcarbodiimide hydrochloride (16.4 g, 85.5 mmol), followed by C44 (21.7 g, 76.4 mmol). After the reaction mixture had been stirred at room temperature for 20 hours, LCMS analysis indicated conversion to C45: LCMS m/z 561.5 [M+H]⁺. The reaction mixture was diluted with water (300 mL) and dichloromethane (200 mL); the aqueous layer was extracted with dichloromethane (2×200 mL) and the combined organic layers were washed with saturated aqueous sodium chloride solution, dried over sodium sulfate, filtered, and concentrated in vacuo. The residue was heated in acetonitrile (250 mL) to provide a solution; upon cooling, a precipitate formed, and the slurry was stirred overnight. Collection of the solids via filtration afforded C45 as a white solid (29.5 g). The mother liquors were concentrated under reduced pressure and treated in the same manner with acetonitrile (50 mL) to afford additional C45 as a white solid (7.50 g). Combined yield: 37.0 g, 66.0 mmol, 86%. ¹H NMR (400 MHz, chloroform-d) δ 9.92 (s, 1H), 7.89 (d, J=7.9 Hz, 1H), 7.66 (d, J=8.6 Hz, 2H), 7.60 (d, J=7.9 Hz, 1H), 7.31 (dd, J=12.2, 2.2 Hz, 1H), 7.24 (d, J=8.8 Hz, 2H, assumed; partially obscured by solvent peak), 7.16 (t, J=8.3 Hz, 1H), 7.02 (dd, J=8.3, 2.2 Hz, 1H), 6.46 (s, 1H), 4.78 (d, J=7.7 Hz, 1H), 3.63-3.54 (m, 1H), 3.48-3.38 (m, 1H), 3.18 (septet, J=6.9 Hz, 1H), 2.67 (dd, J=12.6, 6.2 Hz, 1H), 2.58 (s, 3H), 2.34-2.20 (m, 1H), 2.20-2.09 (m, 1H), 2.00-1.85 (m, 1H), 1.64 (s, 9H), 1.24 (d, J=6.9 Hz, 6H).

### Step 7. Synthesis of 5-{4-[(1-{[3-fluoro-4-(propan-2-yl)phenyl]carbamoyl}-D-prolyl)amino]phenyl}-6-methylpyridine-2-carboxylic acid (27)

Methanesulfonic acid (25.5 mL, 393 mmol) was added to a solution of C45 (22.0 g, 39.2 mmol) in 1,1,1,3,3,3-hexafluoropropan-2-ol (170 mL). After the reaction mixture had been stirred at room temperature for 2 hours, it was treated with propan-2-yl acetate (250 mL); the resulting solid was collected via filtration and dried in vacuo, whereupon it was dissolved in a mixture of butan-2-one/ethyl acetate/n-butanol (1:1:0.1, 250 mL). This solution was treated with saturated aqueous sodium bicarbonate solution (250 mL), and the resulting heterogeneous mixture was stirred overnight at room temperature and filtered. The collected solids were partitioned between butan-2-one/ethyl acetate/n-butanol (1:1:0.1, 250 mL) and water (100 mL), and the resulting mixture was acidified to pH 4 by addition of concentrated hydrochloric acid (approximately 10 mL). After the aqueous layer had been extracted with a mixture of butan-2-one/ethyl acetate/n-butanol (1:1:0.1, 2×50 mL), the combined organic layers were dried over sodium sulfate, filtered, and concentrated in vacuo. The residue was triturated with diethyl ether (150 mL) to provide 5-{4-[(1-{[3-fluoro-4-(propan-2-yl)phenyl]carbamoyl}-D-prolyl)amino]phenyl}-6-methylpyridine-2-carboxylic acid (27) as a white solid. Yield: 17.9 g, 35.5 mmol, 91%. LCMS m/z 505.5 [M+H]⁺. ¹H NMR (400 MHz, DMSO-d₆) δ 10.16 (s, 1H), 8.40 (s, 1H), 7.92 (d, J=7.9 Hz, 1H), 7.79-7.71 (m, 3H), 7.45-7.36 (m, 3H), 7.24 (dd, component of ABX system, J=8.4, 2.1 Hz, 1H), 7.16 (dd, component of ABX system, J=8.7, 8.6 Hz, 1H), 4.48 (dd, J=8.3, 3.7 Hz, 1H), 3.69-3.60 (m, 1H), 3.57-3.47 (m, 1H), 3.06 (septet, J=6.9 Hz, 1H), 2.50 (s, 3H, assumed; largely obscured by solvent peak), 2.27-2.14 (m, 1H), 2.11-1.87 (m, 3H), 1.17 (d, J=6.9 Hz, 6H).

### Example 28

### 3-Fluoro-4′-[(1-{[4-(propan-2-yl)phenyl]carbamoyl}-D-prolyl)amino][1,1′-biphenyl]-4-carboxylic acid (28)

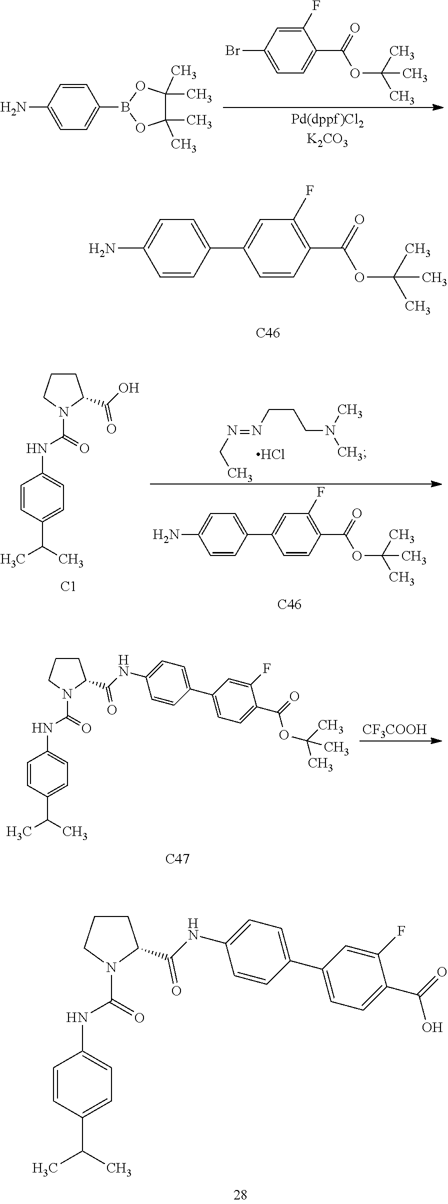

### Step 1. Synthesis of tert-butyl 4′-amino-3-fluoro[1,1′-biphenyl]-4-carboxylate (C46)

To a solution of 4-(4,4,5,5-tetramethyl-1,3,2-dioxaborolan-2-yl)aniline (83.6 g, 382 mmol) in a mixture of 1,4-dioxane (1.6 L) and water (160 mL) were added tert-butyl 4-bromo-2-fluorobenzoate (100 g, 363 mmol) and potassium carbonate (100 g, 724 mmol), followed by [1,1′-bis(diphenylphosphino)ferrocene]dichloropalladium(II) (10.6 g, 14.5 mmol), and the reaction mixture was heated at 85° C. After 4 hours, it was allowed to cool to room temperature and filtered through diatomaceous earth. The filtrate was diluted with water (500 mL), and extracted with ethyl acetate (2×500 mL); the combined organic layers were washed with saturated aqueous sodium chloride solution (2×500 mL), dried over sodium sulfate, filtered, and concentrated in vacuo. Silica gel chromatography (Gradient: 0% to 50% ethyl acetate in petroleum ether) provided material that was then treated with a mixture of petroleum ether and ethyl acetate (5:1, 50 mL), and stirred at 25° C. for 30 minutes. The solid was collected via filtration to provide C46 as a yellow solid (56.8 g). Concentration of the filtrate under reduced pressure, and stirring of the residue in a mixture of petroleum ether and ethyl acetate (4:1, 50 mL) for 30 minutes afforded additional C46 upon filtration, again as a yellow solid (13.2 g). Treatment of this filtrate in similar fashion, but with a 3:1 mixture of petroleum ether and ethyl acetate, gave a third crop of C46 as a yellow solid (15.3 g). Combined yield: 85.3 g, 297 mmol, 82%. LCMS m/z 287.9 [M+H]⁺. ¹H NMR (400 MHz, DMSO-d₆) δ 7.78 (t, J=8.1 Hz, 1H), 7.52-7.43 (m, 4H), 6.64 (d, J=8.6 Hz, 2H), 5.50 (s, 2H), 1.54 (s, 9H).

### Step 2. Synthesis of tert-butyl 3-fluoro-4′-[(1-{[4-(propan-2-yl)phenyl]carbamoyl}-D-prolyl)amino][1,1′-biphenyl]-4-carboxylate (C47)

To a chilled solution (1.1° C. internal temperature) of C1 (10.9 g, 39.4 mmol) in acetonitrile (180 mL) was added 1-[3-(dimethylamino)propyl]-3-ethylcarbodiimide hydrochloride (8.11 g, 42.3 mmol) in a portion-wise manner, such that the internal temperature remained below 1.2° C. over the course of the addition. After 1 hour, C46 (99.4%, 10.4 g, 36.0 mmol) was added portion-wise to the cold reaction mixture, whereupon stirring was continued for 45 minutes before the ice bath was removed; the reaction mixture was then allowed to stir for 5 hours at room temperature. Solids were collected via filtration, and the filter cake was rinsed with acetonitrile (2×60 mL), providing C47 as an off-white solid. Yield: 15.9 g, 29.1 mmol, 81%. LCMS m/z 546.5 [M+H]⁺. ¹H NMR (600 MHz, DMSO-d₆) δ 10.17 (s, 1H), 8.20 (s, 1H), 7.85 (t, J=8.0 Hz, 1H), 7.78-7.72 (m, 4H), 7.64-7.59 (m, 2H), 7.40 (d, J=8.5 Hz, 2H), 7.09 (d, J=8.6 Hz, 2H), 4.47 (dd, J=8.3, 3.7 Hz, 1H), 3.68-3.61 (m, 1H), 3.54-3.48 (m, 1H), 2.80 (septet, J=6.9 Hz, 1H), 2.24-2.14 (m, 1H), 2.08-1.99 (m, 1H), 1.99-1.89 (m, 2H), 1.55 (s, 9H), 1.16 (d, J=6.9 Hz, 6H).

### Step 3. Synthesis of 3-fluoro-4′-[(1-{[4-(propan-2-yl)phenyl]carbamoyl}-D-prolyl)amino][1,1′-biphenyl]-4-carboxylic acid (28)

To a 0° C. solution of C47 (69.5 g, 127 mmol) in dichloromethane (650 mL) was added trifluoroacetic acid (232 g, 2.03 mol). The reaction mixture was allowed to warm to room temperature and stir for 4 hours, whereupon it was concentrated in vacuo, and the residue was diluted with propan-2-yl acetate (400 mL) and stirred for 1 hour. The resulting solid was collected via filtration, rinsed sequentially with propan-2-yl acetate (2×50 mL) and methyl tert-butyl ether (2 25×50 mL), and taken up in a mixture of propan-2-yl acetate (300 mL) and methanol (10 mL). After this mixture had been stirred for 1 hour, it was filtered; the filter cake was rinsed sequentially with propan-2-yl acetate (2×30 mL) and methyl tert-butyl ether (2×40 mL) to provide, after lyophilization to remove residual solvent, 3-fluoro-4′-[(1-{[4-(propan-2-yl)phenyl]carbamoyl}-D-prolyl)amino][1,1′-biphenyl]-4-carboxylic acid (28) as a white solid. Yield: 55.4 g, 113 mmol, 89%. LCMS m/z 490.4 [M+H]⁺. ¹H NMR (400 MHz, DMSO-d₆) δ 13.18 (br s, 1H), 10.17 (s, 1H), 8.20 (s, 1H), 7.91 (t, J=8.3 Hz, 1H), 7.79-7.72 (m, 4H), 7.66-7.58 (m, 2H), 7.40 (d, J=8.6 Hz, 2H), 7.08 (d, J=8.6 Hz, 2H), 4.47 (dd, J=8.3, 3.6 Hz, 1H), 3.70-3.60 (m, 1H), 3.56-3.46 (m, 1H), 2.80 (septet, J=6.9 Hz, 1H), 2.26-2.13 (m, 1H), 2.10-1.87 (m, 3H), 1.16 (d, J=6.9 Hz, 6H).

### Example 29

### 2-Methoxy-4′-[(1-{[4-(propan-2-yl)phenyl]carbamoyl}-D-prolyl)amino][1,1′-biphenyl]-4-carboxylic acid (29)

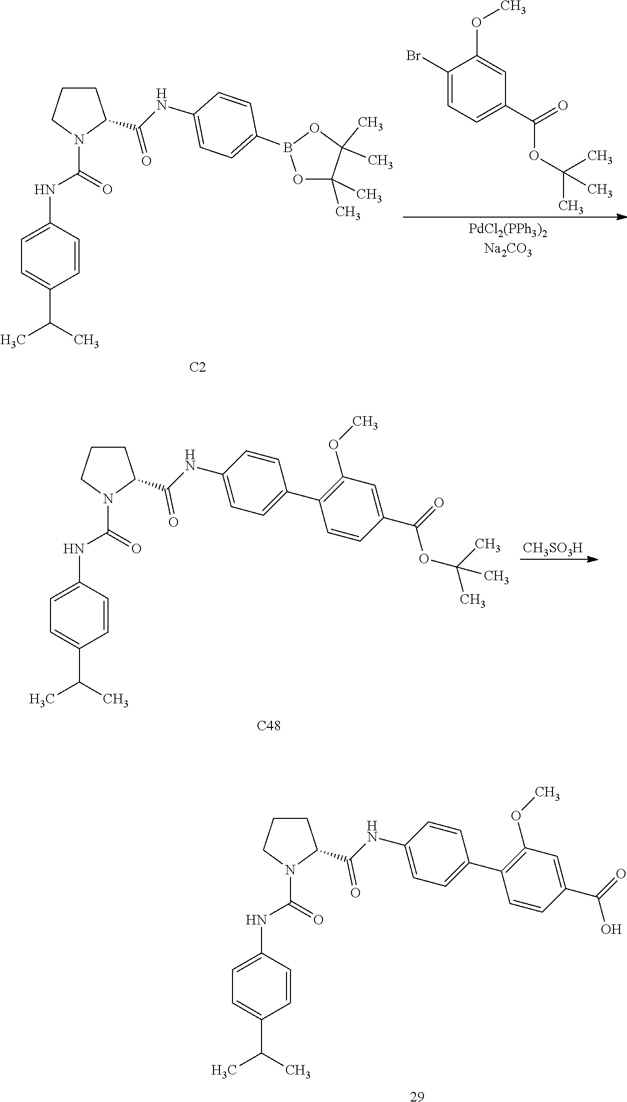

### Step 1. Synthesis of tert-butyl 2-methoxy-4′-[(1-{[4-(propan-2-yl)phenyl]carbamoyl}-D-prolyl)amino][1,1′-biphenyl]-4-carboxylate (C48)

A vial containing tert-butyl 4-bromo-3-methoxybenzoate (474 mg, 1.65 mmol), C2 (750 mg, 1.57 mmol), and bis(triphenylphosphine)palladium(II) dichloride (88.2 mg, 0.126 mmol) was evacuated and charged with nitrogen. This evacuation cycle was repeated twice, whereupon degassed 1,4-dioxane (10.5 mL) and aqueous sodium carbonate solution (2 M; 2.36 mL, 4.72 mmol) were added. The reaction mixture was further degassed with nitrogen for 10 minutes before it was placed in an aluminum heating block and maintained at 90° C. for 3 hours. After it had cooled to room temperature, the reaction mixture was diluted with water (10 mL). The aqueous layer was extracted with ethyl acetate (3×10 mL), and the combined organic layers were washed with saturated aqueous sodium chloride solution (40 mL), dried over magnesium sulfate, filtered, and concentrated in vacuo. Purification via chromatography on silica gel (Gradient: 0% to 50% ethyl acetate in heptane) afforded C48 as a white solid. Yield: 560 mg, 1.00 mmol, 64%. LCMS m/z 558.5 [M+H]⁺. ¹H NMR (400 MHz, chloroform-d) δ 9.87 (s, 1H), 7.62 (dd, J=7.8, 1.6 Hz, 1H), 7.61 (d, J=8.6 Hz, 2H), 7.58 (d, J=1.5 Hz, 1H), 7.48 (d, J=8.6 Hz, 2H), 7.32 (d, J=8.5 Hz, 2H), 7.32 (d, J=7.9 Hz, 1H), 7.20 (d, J=8.4 Hz, 2H), 6.28 (s, 1H), 4.77 (d, J=7.8 Hz, 1H), 3.84 (s, 3H), 3.59-3.51 (m, 1H), 3.47-3.36 (m, 1H), 2.89 (septet, J=6.9 Hz, 1H), 2.68 (br dd, J=12.5, 6.2 Hz, 1H), 2.31-2.19 (m, 1H), 2.19-2.08 (m, 1H), 1.97-1.84 (m, 1H), 1.61 (s, 9H), 1.24 (d, J=6.9 Hz, 6H).

### Step 2. Synthesis of 2-methoxy-4′-[(1-{[4-(propan-2-yl)phenyl]carbamoyl}-D-prolyl)amino][1,1′-biphenyl]-4-carboxylic acid (29)

Methanesulfonic acid (60.5 μL, 0.932 mmol) was added to a solution of C48 (400 mg, 0.717 mmol) in 1,1,1,3,3,3-hexafluoropropan-2-ol (7.2 mL). After 20 minutes at room temperature, the reaction mixture was concentrated in vacuo to remove the 1,1,1,3,3,3-hexafluoropropan-2-ol. The residue was suspended in a mixture of water (5 mL) and acetonitrile (3 mL); the resulting precipitate was collected via filtration, providing 2-methoxy-4′-[(1-{[4-(propan-2-yl)phenyl]carbamoyl}-D-prolyl)amino][1,1′-biphenyl]-4-carboxylic acid (29) as a solid. Yield: 283 mg, 0.564 mmol, 79%. LCMS m/z 502.5 [M+H]⁺. ¹H NMR (400 MHz, methanol-d₄) δ 7.70-7.66 (m, 2H), 7.63 (d, J=8.6 Hz, 2H), 7.49 (d, J=8.7 Hz, 2H), 7.39 (d, J=8.4 Hz, 1H), 7.32 (d, J=8.6 Hz, 2H), 7.13 (d, J=8.4 Hz, 2H), 4.58 (dd, J=8.3, 3.4 Hz, 1H), 3.85 (s, 3H), 3.78-3.69 (m, 1H), 3.63-3.54 (m, 1H), 2.84 (septet, J=6.9 Hz, 1H), 2.37-2.24 (m, 1H), 2.23-2.00 (m, 3H), 1.21 (d, J=6.9 Hz, 6H).

A portion of this material (17 mg) was subjected to reversed-phase HPLC (Column: Waters Sunfire C18, 19×100 mm, 5 μm; Mobile phase A: 0.05% trifluoroacetic acid in water (v/v); Mobile phase B: 0.05% trifluoroacetic acid in acetonitrile (v/v); Gradient: 5% to 95% B over 8.54 minutes, followed by 95% B for 1.46 minutes; Flow rate: 25 mL/minute), affording 2-methoxy-4′-[(1-{[4-(propan-2-yl)phenyl]carbamoyl}-D-prolyl)amino][1,1′-biphenyl]-4-carboxylic acid (29; 12 mg).

### Example 30

### 3-Methoxy-4′-[(1-{[3-methyl-4-(propan-2-yl)phenyl]carbamoyl}-D-prolyl)amino][1,1′-biphenyl]-4-carboxylic acid (30)

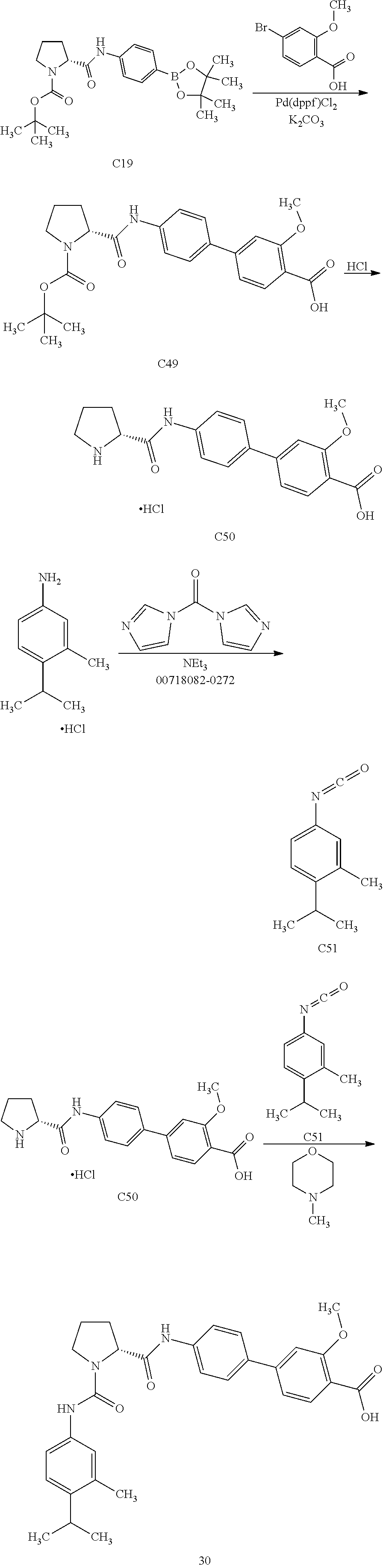

### Step 1. Synthesis of 4′-{[1-(tert-butoxycarbonyl)-D-prolyl]amino}-3-methoxy[1,1′-biphenyl]-4-carboxylic acid (C49)

To a 25° C. solution of C19 (1.00 g, 2.40 mmol) and 4-bromo-2-methoxybenzoic acid (610 mg, 2.64 mmol) in a mixture of 1,4-dioxane (12 mL) and water (3 mL) were added potassium carbonate (996 mg, 7.21 mmol) and [1,1′-bis(diphenylphosphino)ferrocene]dichloropalladium(II) (176 mg, 0.240 mmol), whereupon the reaction mixture was degassed with nitrogen for 1 minute and heated to 100° C. After 16 hours, LCMS analysis indicated conversion to C49: LCMS m/z 441.3 [M+H]⁺. The reaction mixture was cooled and filtered; the filtrate was diluted with water (50 mL), acidified to a pH of approximately 6 by addition of concentrated hydrochloric acid, and extracted with ethyl acetate (2×50 mL). The combined organic layers were washed with saturated aqueous sodium chloride solution (2×50 mL), dried over sodium sulfate, filtered, and concentrated in vacuo to provide C49 as a yellow solid (1.06 g). Most of this material was used directly in the next step.

### Step 2. Synthesis of 3-methoxy-4′-(D-prolylamino)[1,1′-biphenyl]-4-carboxylic acid, hydrochloride salt (C50)

To a solution of C49 (from the previous step; 1.00 g, ≤2.26 mmol) in dichloromethane (8 mL) was added a solution of hydrogen chloride in 1,4-dioxane (4 M; 4 mL, 16 mmol). After the reaction mixture had been stirred at 25° C. for 2 hours, it was concentrated in vacuo, diluted with dichloromethane (15 mL), and concentrated under reduced pressure; this dilution/concentration was carried out a total of 3 times. The resulting solid was stirred with a mixture of dichloromethane and ethyl acetate (1:1, 20 mL) for 30 minutes, whereupon the mixture was filtered. The filter cake was washed with ethyl acetate (10 mL) to afford C50 as a yellow solid (850 mg). A portion of this material was used in Step 4. LCMS m/z 341.2 [M+H]⁺.

### Step 3. Synthesis of 4-isocyanato-2-methyl-1-(propan-2-yl)benzene (C51)

Triethylamine (37.5 μL, 0.269 mmol) was added to a solution of 3-methyl-4-(propan-2-yl)aniline, hydrochloride salt (50 mg, 0.27 mmol) in acetonitrile (2 mL); after the mixture had been stirred at 25° C. for 5 minutes, 1,1′-carbonyldiimidazole (43.7 mg, 0.269 mmol) was added, and the reaction mixture was stirred at 25° C. for 1 hour. Removal of solvent in vacuo provided crude C51 as a brown solid, which was progressed directly to the following step.

### Step 4. Synthesis of 3-methoxy-4′-[(1-{[3-methyl-4-(propan-2-yl)phenyl]carbamoyl}-D-prolyl)amino][1,1′-biphenyl]-4-carboxylic acid (30)

To a solution of C50 (from Step 2; 101 mg, 50.268 mmol) in tetrahydrofuran (2.0 mL) was added 4-methylmorpholine (0.147 mL, 1.34 mmol). The mixture was stirred at 25° C. for 5 minutes, whereupon C51 (from the previous step; 50.27 mmol) was added, and stirring was continued at 25° C. for 3 days. After the reaction mixture had been diluted with water (30 mL), it was acidified to a pH of approximately 4 by addition of 1 M hydrochloric acid and extracted with ethyl acetate (2×30 mL). The combined organic layers were washed with saturated aqueous sodium chloride solution (2×50 mL), dried over sodium sulfate, filtered, and concentrated in vacuo; purification via reversed-phase HPLC (Column: C18, 40×150 mm; Mobile phase A: water containing 0.05% ammonium hydroxide and 10 mM ammonium bicarbonate in water; Mobile phase B: acetonitrile; Gradient: 8% to 48% B; Flow rate: 60 mL/min) afforded 3-methoxy-4′-[(1-{[3-methyl-4-(propan-2-yl)phenyl]carbamoyl}-D-prolyl)amino][1,1′-biphenyl]-4-carboxylic acid (30) as a white solid. Yield: 32.6 mg, 63.2 μmol, 23% over 3 steps. LCMS m/z 516.4 [M+H]⁺. ¹H NMR (400 MHz, DMSO-d₆) δ 10.14 (s, 1H), 8.12 (s, 1H), 7.73 (s, 4H), 7.70 (d, J=8.0 Hz, 1H), 7.31 (d, J=1.6 Hz, 1H), 7.30-7.24 (m, 3H), 7.09-7.04 (m, 1H), 4.47 (dd, J=8.4, 3.6 Hz, 1H), 3.91 (s, 3H), 3.68-3.59 (m, 1H), 3.55-3.46 (m, 1H), 3.01 (septet, J=6.9 Hz, 1H), 2.22 (s, 3H), 2.22-2.13 (m, 1H), 2.09-1.88 (m, 3H), 1.13 (d, J=6.8 Hz, 6H).

### Example 31

### 4-{6-[(1-{[4-(Trifluoromethyl)phenyl]carbamoyl}-D-prolyl)amino]pyridin-3-yl}benzoic acid (31)

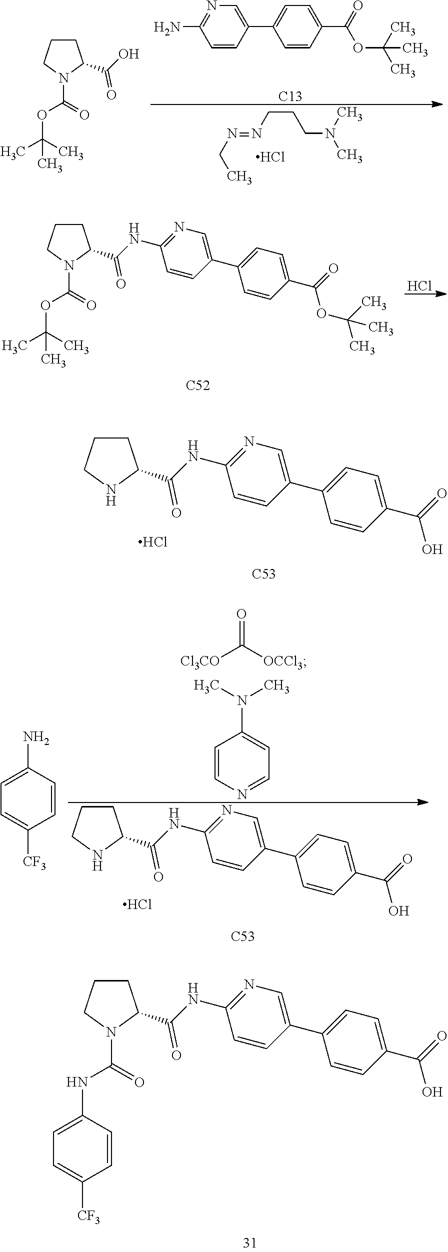

### Step 1. Synthesis of tert-butyl (2R)-2-({5-[4-(tert-butoxycarbonyl)phenyl]pyridin-2-yl}carbamoyl)pyrrolidine-1-carboxylate (C52)

A solution of 1-(tert-butoxycarbonyl)-D-proline (900 mg, 4.18 mmol), C13 (1.13 g, 4.18 mmol), and 1-[3-(dimethylamino)propyl]-3-ethylcarbodiimide hydrochloride (1.04 g, 5.42 mmol) in dichloromethane (21 mL) was stirred at room temperature overnight. The reaction mixture was then diluted with dichloromethane and washed sequentially with water and saturated aqueous sodium chloride solution, dried over sodium sulfate, filtered, and concentrated in vacuo. Silica gel chromatography (Gradient: 0% to 100% ethyl acetate in heptane) provided C52 as a white solid; by ¹H NMR analysis, this material exists as a mixture of rotamers. Yield: 1.39 g, 2.97 mmol, 71%. LCMS m/z 468.5 [M+H]⁺. ¹H NMR (400 MHz, DMSO-d₆) δ [10.76 (s) and 10.70 (s), total 1H], 8.73 (br s, 1H), 8.25-8.14 (m, 2H), 7.92 (AB quartet, J_{AB}=8.5 Hz, Δv_{AB}=47.2 Hz, 4H), [4.49-4.42 (m) and 4.40 (dd, J=8.3, 4.2 Hz), total 1H], 3.48-3.3 (m, 2H, assumed; partially obscured by water peak), 2.29-2.10 (m, 1H), 1.96-1.73 (m, 3H), 1.57 (s, 9H), [1.40 (s) and 1.27 (s), total 9H].

### Step 2. Synthesis of 4-[6-(D-prolylamino)pyridin-3-yl]benzoic acid, hydrochloride salt (C53)

A solution of hydrogen chloride in 1,4-dioxane (4.0 M; 20 mL, 80 mmol) was added to a solution of C52 (1.39 g, 2.97 mmol) in 1,4-dioxane (20 mL), whereupon the reaction mixture was stirred at room temperature overnight. A solution of hydrogen chloride in 1,4-dioxane (4.0 M; 20 mL, 80 mmol) was again added; after the reaction mixture had been stirred overnight once more, solids were collected via filtration. The filter cake was washed with 1,4-dioxane to afford C53 as a white solid (1.16 g). A portion of this material was used in the following experiment. LCMS m/z 312.2 [M+H]⁺. ¹H NMR (400 MHz, DMSO-d₆) δ 11.32 (s, 1H), 10.11-9.98 (m, 1H), 8.79 (br d, J=2.5 Hz, 1H), 8.76-8.65 (m, 1H), 8.26 (dd, J=8.7, 2.5 Hz, 1H), 8.16 (br d, J=8.7 Hz, 1H), 8.04 (d, J=8.5 Hz, 2H), 7.88 (d, J=8.5 Hz, 2H), 4.52-4.40 (m, 1H), 3.36-3.20 (m, 2H), 2.48-2.37 (m, 1H), 2.06-1.88 (m, 3H).

### Step 3. Synthesis of 4-{6-[(1-{[4-(trifluoromethyl)phenyl]carbamoyl}-D-prolyl)amino]pyridin-3-yl}benzoic acid (31)

A solution of 4-(trifluoromethyl)aniline (169 mg, 1.05 mmol) in dichloromethane (11.5 mL) was treated with a solution of bis(trichloromethyl) carbonate (109 mg, 0.367 mmol) in dichloromethane (1 mL), followed by 4-(dimethylamino)pyridine (639 mg, 5.23 mmol). After the reaction mixture had been stirred for 1 hour at room temperature, C53 (from the previous step; 450 mg, 51.15 mmol) was added in one portion, and stirring was continued at room temperature for 1 hour. The reaction mixture was acidified to a pH of approximately 3 by addition of 1 M hydrochloric acid, and then extracted with a 2:2:1 mixture of ethyl acetate/butan-2-one/butan-2-ol; the organic layer was washed with saturated aqueous sodium chloride solution, dried over sodium sulfate, filtered, and concentrated in vacuo. The resulting material was triturated in diethyl ether, then treated with a small amount of 10% dichloromethane in methanol and stirred for 30 minutes; collection of the resulting solid via filtration provided 4-{6-[(1-{[4-(trifluoromethyl)phenyl]carbamoyl}-D-prolyl)amino]pyridin-3-yl}benzoic acid (31) as a white solid (124 mg). The filtrate was concentrated almost to dryness, treated with a small amount of 10% dichloromethane in methanol and stirred for 30 minutes. Filtration provided a second batch of 4-{6-[(1-{[4-(trifluoromethyl)phenyl]carbamoyl}-D-prolyl)amino]pyridin-3-yl}benzoic acid (31) as a white solid (24 mg). Combined yield: 148 mg, 0.297 mmol, 26% over 2 steps. LCMS m/z 499.4 [M+H]⁺. ¹H NMR (400 MHz, DMSO-d₆) δ 12.99 (br s, 1H), 10.72 (s, 1H), 8.74 (t, J=1.7 Hz, 1H), 8.71 (s, 1H), 8.18 (d, J=1.7 Hz, 2H), 8.02 (d, J=8.5 Hz, 2H), 7.86 (d, J=8.5 Hz, 2H), 7.75 (d, J=8.5 Hz, 2H), 7.58 (d, J=8.6 Hz, 2H), 4.69-4.61 (m, 1H), 3.72-3.63 (m, 1H), 3.62-3.53 (m, 1H), 2.28-2.15 (m, 1H), 2.09-1.90 (m, 3H).

### Example 32

### 4′-[(1-{[3-Fluoro-4-(propan-2-yl)phenyl]carbamoyl}-D-prolyl)amino]-3-methoxy[1,1′-biphenyl]-4-carboxylic acid (32)

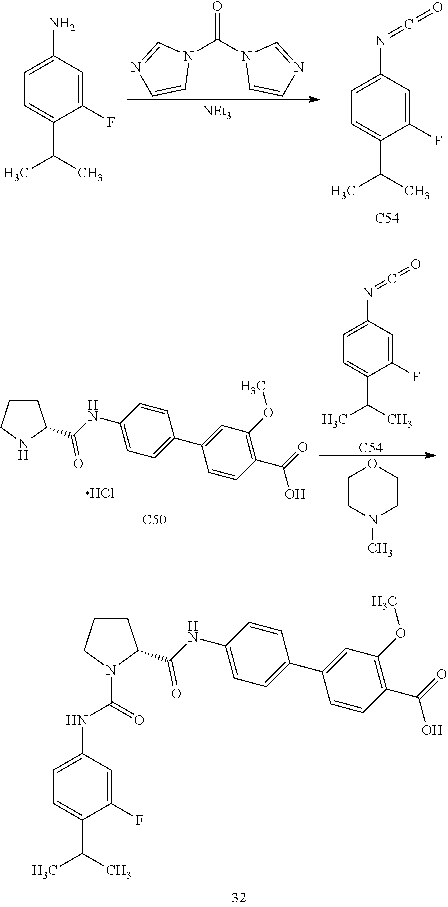

### Step 1. Synthesis of 2-fluoro-4-isocyanato-1-(propan-2-yl)benzene (C54)

Triethylamine (54.6 μL, 0.392 mmol) was added to a solution of 3-fluoro-4-(propan-2-yl)aniline (50 mg, 0.33 mmol) in acetonitrile (2 mL); after the mixture had been stirred for 10 minutes, 1,1′-carbonyldiimidazole (52.9 mg, 0.326 mmol) was added, and stirring was continued for an additional 10 minutes, whereupon the reaction mixture was concentrated in vacuo. The resulting crude C54 was used directly in the following step.

### Step 2. Synthesis of 4′-[(1-{[3-fluoro-4-(propan-2-yl)phenyl]carbamoyl}-D-prolyl)amino]-3-methoxy[1,1′-biphenyl]-4-carboxylic acid (32)

To a suspension of C50 (from Example 30, Step 2; 122 mg, ≤0.324 mmol) in tetrahydrofuran (1 mL) was added 4-methylmorpholine (0.214 mL, 1.95 mmol); after 10 minutes at 25° C., a solution of C54 (from the previous step; ≤0.33 mmol) in tetrahydrofuran (1 mL) was added, and stirring was continued at 25° C. for 1 hour. Water (5 mL) was added to the reaction mixture, whereupon the mixture was poured into water (10 mL) and extracted with ethyl acetate (3×5 mL). The combined organic layers were washed with saturated aqueous sodium chloride solution (10 mL), dried over sodium sulfate, filtered, and concentrated in vacuo. Purification via reversed-phase HPLC (Column: C18, 40×150 mm; Mobile phase A: water containing 0.05% ammonium hydroxide and 10 mM ammonium bicarbonate in water; Mobile phase B: acetonitrile; Gradient: 8% to 48% B; Flow rate: 60 mL/min) afforded 4′-[(1-{[3-fluoro-4-(propan-2-yl)phenyl]carbamoyl}-D-prolyl)amino]-3-methoxy[1,1′-biphenyl]-4-carboxylic acid (32) as a white solid. Yield: 9.3 mg, 17.9 μmol, 6% over 3 steps. LCMS m/z 520.3 [M+H]⁺. ¹H NMR (400 MHz, DMSO-d₆) δ 10.16 (s, 1H), 8.41 (s, 1H), 7.73 (s, 4H), 7.71 (d, J=8.0 Hz, 1H), 7.41 (dd, J=13.7, 2.1 Hz, 1H), 7.32 (d, J=1.6 Hz, 1H), 7.29-7.22 (m, 2H), 7.16 (t, J=8.7 Hz, 1H), 4.48 (dd, J=8.3, 3.7 Hz, 1H), 3.91 (s, 3H), 3.69-3.60 (m, 1H), 3.56-3.47 (m, 1H), 3.13-3.00 (m, 1H), 2.27-2.14 (m, 1H), 2.10-1.88 (m, 3H), 1.17 (d, J=6.9 Hz, 6H).

### Example 33

### 4-{3-Fluoro-5-[(1-{[4-(propan-2-yl)phenyl]carbamoyl}-D-prolyl)amino]pyridin-2-yl}benzoic acid (33)

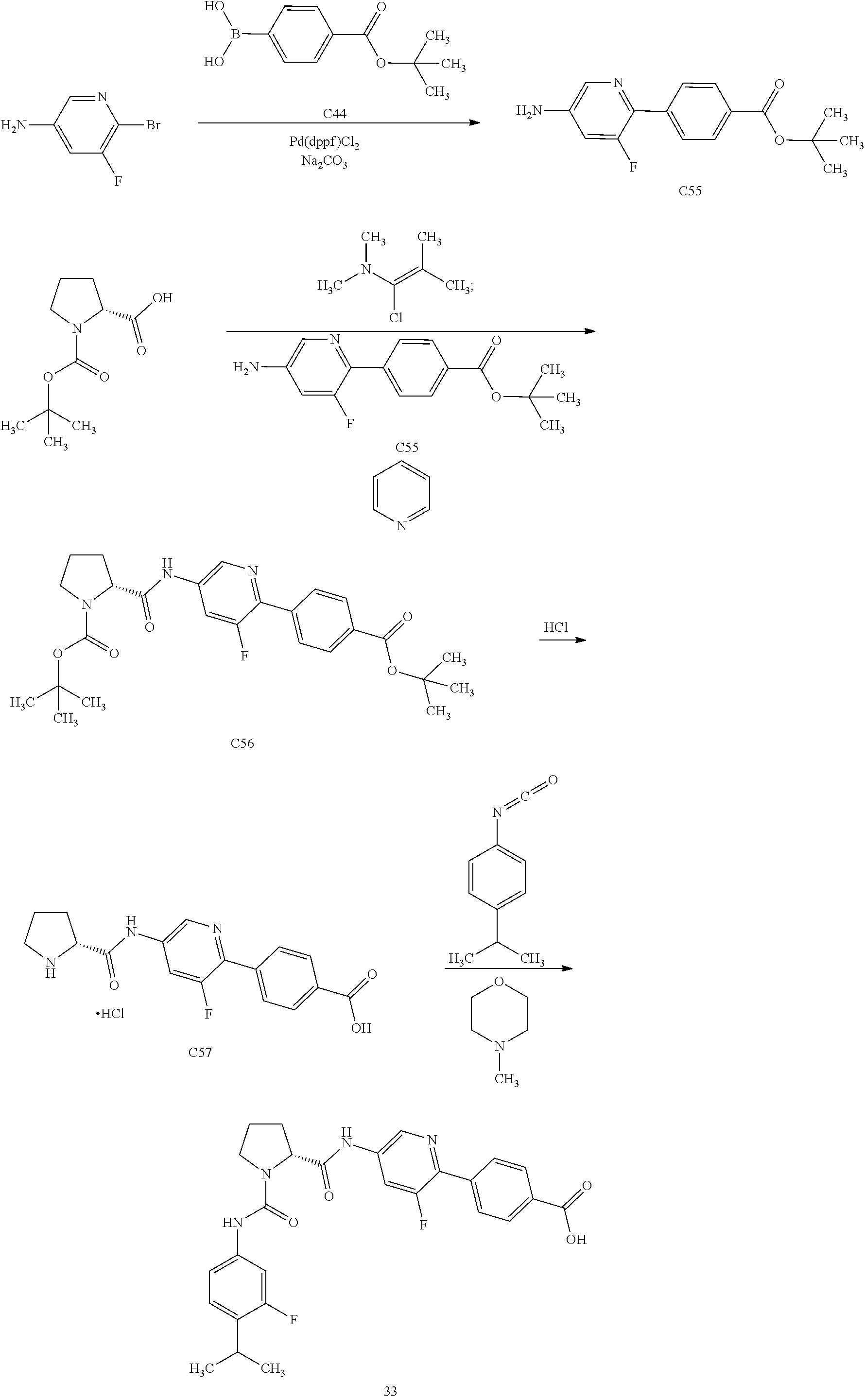

### Step 1. Synthesis of tert-butyl 4-(5-amino-3-fluoropyridin-2-yl)benzoate (C55)

A mixture of 6-bromo-5-fluoropyridin-3-amine (500 mg, 2.62 mmol), [4-(tert-butoxycarbonyl)phenyl]boronic acid (639 mg, 2.88 mmol), sodium carbonate (832 mg, 7.85 mmol), and [1,1′-bis(diphenylphosphino)ferrocene]dichloropalladium(II) (192 mg, 0.262 mmol) in a mixture of 1,4-dioxane (10 mL) and water (3 mL) was heated at 80° C. for 3 hours. After removal of 1,4-dioxane by concentration under reduced pressure, the residue was partitioned between ethyl acetate and water. The organic layer was washed with saturated aqueous sodium chloride solution, dried over magnesium sulfate, filtered, and concentrated in vacuo; silica gel chromatography (Gradient: 30% to 50% ethyl acetate in heptane) provided C55 as a pale-yellow oil. Yield: 720 mg, 2.50 mmol, 95%. LCMS m/z 289.2 [M+H]⁺. ¹H NMR (400 MHz, DMSO-d₆) δ 8.00-7.88 (m, 5H), 6.82 (dd, J=14.4, 2.2 Hz, 1H), 6.01 (br s, 2H), 1.56 (s, 9H).

### Step 2. Synthesis of tert-butyl (2R)-2-({6-[4-(tert-butoxycarbonyl)phenyl]-5-fluoropyridin-3-yl}carbamoyl)pyrrolidine-1-carboxylate (C56)

To a solution of 1-(tert-butoxycarbonyl)-D-proline (400 mg, 1.86 mmol) in dichloromethane (5.0 mL) was added 1-chloro-N,N,2-trimethylprop-1-en-1-amine (250 μL, 1.86 mmol), whereupon the reaction mixture was allowed to stir for 30 minutes. A solution of C55 (536 mg, 1.86 mmol) in dichloromethane (5 mL) was then added, followed by pyridine (450 μL, 5.6 mmol), and stirring was continued at room temperature for 1 hour. The reaction mixture was partitioned between dichloromethane and water, and the organic layer was washed with saturated aqueous sodium chloride solution, dried over magnesium sulfate, filtered, and concentrated in vacuo. Chromatography on silica gel (Gradient: 30% to 50% ethyl acetate in heptane) afforded C56 as a colorless oil. Yield: 800 mg, 1.65 mmol, 89%. LCMS m/z 484.4 [M−H]⁻. ¹H NMR (400 MHz, DMSO-d₆), characteristic peaks; integrations are approximate: δ 10.65 (br s, 1H), 8.24-8.17 (m, 1H), 8.06-7.99 (m, 4H), [4.30 (dd, J=8.4, 3.1 Hz) and 4.24 (dd, J=8.2, 4.4 Hz), total 1H], 3.49-3.33 (m, 2H), 2.32-2.14 (m, 1H), 1.99-1.76 (m, 3H), 1.57 (s, 9H), [1.41 (s) and 1.28 (s), total 9H].

### Step 3. Synthesis of 4-[3-fluoro-5-(D-prolylamino)pyridin-2-yl]benzoic acid, hydrochloride salt (C57)

A solution of hydrogen chloride in 1,4-dioxane (4 M; 5 mL, 20 mmol) was added to C56 (660 mg, 1.36 mmol) with stirring, and the reaction mixture was stirred at room temperature for 1 hour. The resulting solids were broken up manually, and the reaction mixture was heated at 50° C. for 30 minutes, whereupon it was concentrated in vacuo. The residue was stirred in diethyl ether for 5 minutes and filtered; the filter cake was washed with diethyl ether to provide C57 (579 mg; this material contained 1,4-dioxane and diethyl ether). Yield, corrected for solvents: 399 mg, 1.09 mmol, 80%. LCMS m/z 330.3 [M+H]⁺. ¹H NMR (400 MHz, DMSO-d₆) δ 11.86 (s, 1H), 10.20-10.04 (m, 1H), 8.84 (t, J=1.7 Hz, 1H), 8.83-8.74 (m, 1H), 8.20 (dd, J=13.4, 2.0 Hz, 1H), 8.05 (AB quartet, upfield doublet is broadened, J_{AB}=8.6 Hz, Δv_{AB}=12.1 Hz, 4H), 4.56-4.43 (m, 1H), 3.34-3.21 (m, 2H), 2.5-2.40 (m, 1H, assumed; partially obscured by solvent peak), 2.09-1.88 (m, 3H).

### Step 4. Synthesis of 4-{3-fluoro-5-[(1-{[4-(propan-2-yl)phenyl]carbamoyl}-D-prolyl)amino]pyridin-2-yl}benzoic acid (33)

4-Methylmorpholine (50 μL, 0.45 mmol) was added to a solution of C57 (150 mg, 0.410 mmol) in N,N-dimethylacetamide (2 mL). After the resulting solution had been stirred for 2 minutes, 1-isocyanato-4-(propan-2-yl)benzene (66.1 mg, 0.410 mmol) was added. Stirring was continued at room temperature for 20 minutes, whereupon the reaction mixture was diluted with water (20 mL), stirred for 10 minutes and filtered. The filter cake was washed with water, then purified via reversed-phase HPLC (Column: Waters Sunfire C18, 19×100 mm, 5 μm; Mobile phase A: 0.05% trifluoroacetic acid in water (v/v); Mobile phase B: 0.05% trifluoroacetic acid in acetonitrile (v/v); Gradient: 25% to 65% B over 8.5 minutes, then 65% to 95% B over 0.5 minutes, then 95% B for 1.0 minute; Flow rate: 25 mL/minute) to afford 4-{3-fluoro-5-[(1-{[4-(propan-2-yl)phenyl]carbamoyl}-D-prolyl)amino]pyridin-2-yl}benzoic acid (33). Yield: 37 mg, 75 μmol, 18%. LCMS m/z 491.3 [M+H]⁺. ¹H NMR (600 MHz, DMSO-d₆) δ 10.65 (s, 1H), 8.71 (br s, 1H), 8.24 (s, 1H), 8.20 (dd, J=13.7, 2.0 Hz, 1H), 8.04 (AB quartet, J_{AB}=8.3 Hz, Δv_{AB}=18.1 Hz, 4H), 7.39 (d, J=8.4 Hz, 2H), 7.09 (d, J=8.5 Hz, 2H), 4.47 (dd, J=8.3, 3.9 Hz, 1H), 3.68-3.62 (m, 1H), 3.56-3.48 (m, 1H, assumed; partially obscured by water peak), 2.81 (septet, J=6.9 Hz, 1H), 2.27-2.18 (m, 1H), 2.08-1.91 (m, 3H), 1.16 (d, J=6.9 Hz, 6H).

### Example 34

### 4′-{[(3S)-3-Methyl-1-{[4-(propan-2-yl)phenyl]carbamoyl}-D-prolyl]amino}[1,1′-biphenyl]-4-carboxylic acid or 4′-{[(3R)-3-Methyl-1-{[4-(propan-2-yl)phenyl]carbamoyl}-L-prolyl]amino}[1,1′-biphenyl]-4-carboxylic acid {34 [from C59 (DIAST-1)]}

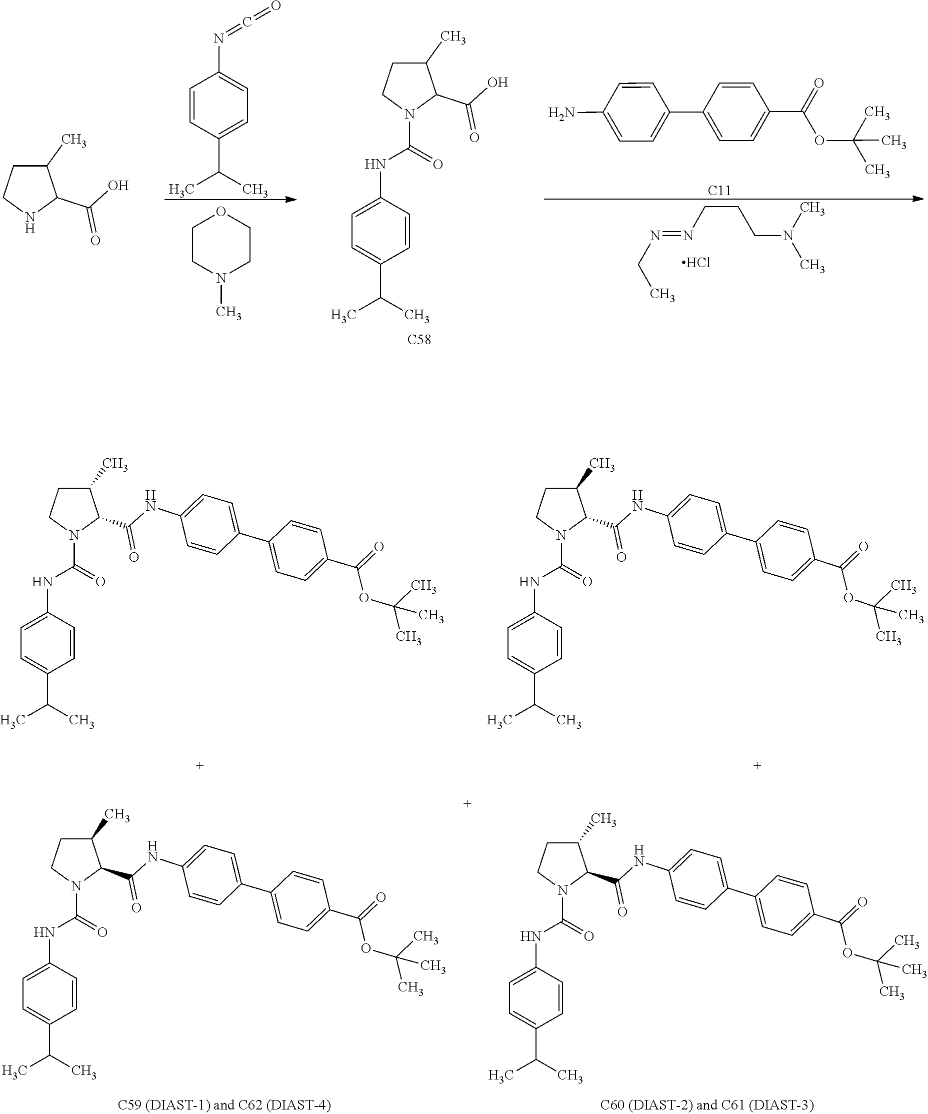

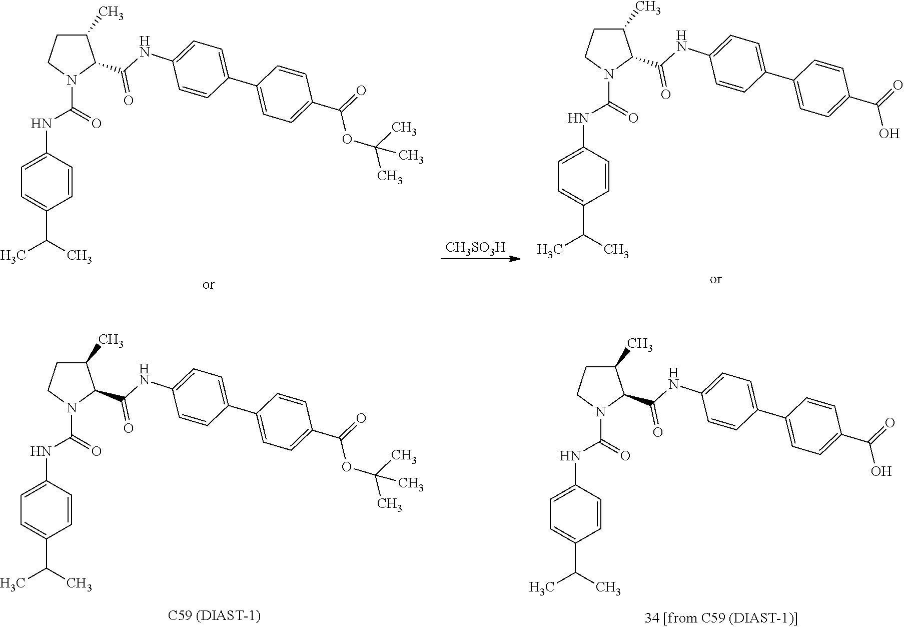 [SMILES: CC(C)c1ccc(NC(=O)N2CC[C@H](C)[C@@H]2C(=O)Nc2ccc(-c3ccc(C(=O)OC(C)(C)C)cc3)cc2)cc1 ; "CH3SO3H" ; CC(C)c1ccc(NC(=O)N2CC[C@H](C)[C@@H]2C(=O)Nc2ccc(-c3ccc(C(=O)O)cc3)cc2)cc1 ; CC(C)c1ccc(NC(=O)N2CC[C@@H](C)[C@H]2C(=O)Nc2ccc(-c3ccc(C(=O)OC(C)(C)C)cc3)cc2)cc1 ; CC(C)c1ccc(NC(=O)N2CC[C@@H](C)[C@H]2C(=O)Nc2ccc(-c3ccc(C(=O)O)cc3)cc2)cc1]

### Step 1. Synthesis of 3-methyl-1-{[4-(propan-2-yl)phenyl]carbamoyl}pyrrolidine-2-carboxylic acid (C58)

4-Methylmorpholine (1.28 mL, 11.6 mmol) and 1-isocyanato-4-(propan-2-yl)benzene (624 mg, 3.87 mmol) were added to a solution of 3-methylpyrrolidine-2-carboxylic acid (500 mg, 3.87 mmol) in tetrahydrofuran (19 mL), and the reaction mixture was stirred at 20° C. for 2 hours. After concentration under reduced pressure, the residue was diluted with water (20 mL), treated with sodium bicarbonate (488 mg, 5.81 mmol), and washed with methyl tert-butyl ether (2×10 mL). The aqueous layer was then acidified to a pH of approximately 2 by addition of 1 M hydrochloric acid; the resulting suspension was extracted with ethyl acetate (2×15 mL). The ethyl acetate layers were combined, dried over sodium sulfate, filtered, and concentrated in vacuo to provide a solid, which was triturated with a mixture of petroleum ether and ethyl acetate (3:1, 24 mL) for 15 minutes to afford C58 as a white solid; this material comprised a mixture of diastereomers. Yield: 491 mg, 1.69 mmol, 44%. LCMS m/z 291.0 [M+H]⁺. ¹H NMR (400 MHz, DMSO-d₆) δ 12.35 (br s, 1H), [8.17 (s) and 8.11 (s), total 1H], 7.37 (d, J=8.6 Hz, 2H), 7.08 (d, J=8.6 Hz, 2H), [4.22 (d, J=8.3 Hz) and 3.83 (d, J=5.7 Hz), total 1H], 3.68-3.36 (m, 2H), 2.80 (septet, J=6.9 Hz, 1H), [2.5-2.41 (m) and 2.29-2.17 (m), total 1H, assumed; partially obscured by solvent peak], 2.12-1.97 (m, 1H), 1.75-1.53 (m, 1H), 1.16 (d, J=6.9 Hz, 6H), [1.11 (d, J=6.8 Hz) and 0.99 (d, J=6.9 Hz), total 3H].

### Step 2. Synthesis of tert-butyl 4′-{[(3S)-3-methyl-1-{[4-(propan-2-yl)phenyl]carbamoyl}-D-prolyl]amino}[1,1′-biphenyl]-4-carboxylate and tert-butyl 4′-{[(3R)-3-methyl-1-{[4-(propan-2-yl)phenyl]carbamoyl}-L-prolyl]amino}[1,1′-biphenyl]-4-carboxylate [C59 (DIAST-1) and C62 (DIAST-4)]; and tert-butyl 4′-{[(3R)-3-methyl-1-{[4-(propan-2-yl)phenyl]carbamoyl}-D-prolyl]amino}[1,1′-biphenyl]-4-carboxylate and tert-butyl 4′-{[(3S)-3-methyl-1-{[4-(propan-2-yl)phenyl]carbamoyl}-L-prolyl]amino}[1,1′-biphenyl]-4-carboxylate [C60 (DIAST-2) and C61 (DIAST-3)]

To a solution of C58 (300 mg, 1.03 mmol) and C11 (278 mg, 1.03 mmol) in dichloromethane (5 mL) was added 1-[3-(dimethylamino)propyl]-3-ethylcarbodiimide hydrochloride (257 mg, 1.34 mmol). After the reaction mixture had been stirred at 25° C. for 2 hours, it was poured into water (20 mL) and extracted with dichloromethane (2×10 mL). The combined organic layers were washed with saturated aqueous sodium chloride solution (2×25 mL), dried over sodium sulfate, filtered, and concentrated in vacuo. The residue was stirred with a mixture of ethyl acetate and petroleum ether (1:2, 15 mL) for 10 minutes before being filtered; the collected solid contained a mixture of the four diastereomeric products as a white solid. Yield: 448 mg, 0.827 mmol, 80%. LCMS m/z 542.5 [M+H]⁺.

Separation of the individual isomers was carried out using supercritical fluid chromatography [Column: Regis Technologies, (S,S)-Whelk-O 1, 25×250 mm, 10 μm; Mobile phase: 45:55 carbon dioxide/(ethanol containing 0.1% ammonium hydroxide); Flow rate: 80 mL/minute], providing C59 (DIAST-1), C60 (DIAST-2), C61 (DIAST-3), and C62 (DIAST-4). All four products were obtained as yellow solids.

The indicated relative stereochemistries were assigned on the basis of NOE studies carried out using C61 (DIAST-3), and C62 (DIAST-4). In C62 (DIAST-4), the pyrrolidine proton geminal to the amide carbonyl exhibits an NOE interaction with the adjacent methine proton that is stronger than its interaction with the adjacent methyl group, indicating a cis configuration of the two substituents on the pyrrolidine. The interaction with that methyl group is stronger for the corresponding proton in C61 (DIAST-3), supporting a trans assignment. Comparison of the four NMR spectra allowed assignment of C59 (DIAST-1) and C62 (DIAST-4) as enantiomers of one another; C60 (DIAST-2) is the enantiomer of C61 (DIAST-3).

C59 (DIAST-1)—Yield: 61.0 mg, 0.113 mmol, 14% for the separation. Retention time: 1.73 minutes; Analytical conditions [Column: Regis Technologies, (S,S)-Whelk-O 1, 4.6×100 mm, 5.0 μm; Mobile phase: 1:1 carbon dioxide/(methanol containing 0.05% diethylamine); Flow rate: 2.5 mL/minute; Back pressure: 100 bar]. ¹H NMR (400 MHz, chloroform-d) δ 9.79 (br s, 1H), 7.91 (d, J=8.4 Hz, 2H), 7.44 (d, J=8.6 Hz, 2H), 7.36 (d, J=8.5 Hz, 2H), 7.36 (d, J=8.4 Hz, 2H), 7.20 (d, J=8.4 Hz, 2H), 7.12 (d, J=8.5 Hz, 2H), 6.38 (s, 1H), 4.86 (d, J=8.0 Hz, 1H), 3.84 (t, J=8.3 Hz, 1H), 3.58-3.49 (m, 1H), 2.84 (septet, J=6.9 Hz, 1H), 2.59-2.48 (m, 1H), 2.35-2.20 (m, 1H), 2.19-2.09 (m, 1H), 1.60 (s, 9H), 1.20 (d, J=6.9 Hz, 6H), 1.16 (d, J=6.8 Hz, 3H).
C60 (DIAST-2) -Yield: 76 mg, 0.140 mmol, 17% for the separation. Retention time: 2.26 minutes; Analytical conditions identical to those used for C59. ¹H NMR (400 MHz, chloroform-d) δ 9.80 (s, 1H), 7.98 (d, J=8.1 Hz, 2H), 7.59 (d, J=8.4 Hz, 2H), 7.51 (d, J=8.1 Hz, 2H), 7.43 (d, J=8.2 Hz, 2H), 7.34 (d, J=8.2 Hz, 2H), 7.16 (d, J=8.1 Hz, 2H), 6.40 (s, 1H), 4.36 (d, J=3.3 Hz, 1H), 3.60 (t, J=6.7 Hz, 2H), 2.94-2.79 (m, 2H), 2.43-2.29 (m, 1H), 1.83-1.7 (m, 1H, assumed; partially obscured by water peak), 1.61 (s, 9H), 1.22 (d, J=6.9 Hz, 6H), 1.14 (d, J=7.0 Hz, 3H).
C61 (DIAST-3)—Yield: 54 mg, 0.10 mmol, 12% for the separation. Retention time: 3.67 minutes; Analytical conditions identical to those used for C59. ¹H NMR (400 MHz, chloroform-d) δ 9.76 (s, 1H), 7.99 (d, J=8.4 Hz, 2H), 7.60 (d, J=8.6 Hz, 2H), 7.54 (d, J=8.4 Hz, 2H), 7.47 (d, J=8.5 Hz, 2H), 7.34 (d, J=8.4 Hz, 2H), 7.17 (d, J=8.5 Hz, 2H), 6.35 (s, 1H), 4.36 (d, J=2.9 Hz, 1H), 3.63-3.55 (m, 2H), 2.95-2.81 (m, 2H), 2.43-2.31 (m, 1H), 1.83-1.72 (m, 1H), 1.61 (s, 9H), 1.23 (d, J=6.9 Hz, 6H), 1.15 (d, J=7.1 Hz, 3H).
C62 (DIAST-4)—Yield: 52 mg, 96 μmol, 12% for the separation. Retention time: 4.45 minutes; Analytical conditions identical to those used for C59. ¹H NMR (400 MHz, chloroform-d) δ 9.86 (br s, 1H), 7.90 (d, J=8.1 Hz, 2H), 7.43 (d, J=8.3 Hz, 2H), 7.39-7.32 (m, 4H), 7.18 (d, J=8.3 Hz, 2H), 7.12 (d, J=8.2 Hz, 2H), 6.39 (s, 1H), 4.88 (d, J=8.0 Hz, 1H), 3.85 (t, J=8.4 Hz, 1H), 3.59-3.49 (m, 1H), 2.84 (septet, J=6.9 Hz, 1H), 2.60-2.46 (m, 1H), 2.36-2.20 (m, 1H), 2.19-2.09 (m, 1H), 1.61 (s, 9H), 1.20 (d, J=6.9 Hz, 6H), 1.16 (d, J=6.7 Hz, 3H).

### Step 3. Synthesis of 4′-{[(3S)-3-methyl-1-{[4-(propan-2-yl)phenyl]carbamoyl}-D-prolyl]amino}[1,1′-biphenyl]-4-carboxylic acid or 4′-{[(3R)-3-methyl-1-{[4-(propan-2-yl)phenyl]carbamoyl}-L-prolyl]amino}[1,1′-biphenyl]-4-carboxylic acid {34 [from C59 (DIAST-1)]}

To a solution of tert-butyl 4′-{[(3S)-3-methyl-1-{[4-(propan-2-yl)phenyl]carbamoyl}-D-prolyl]amino}[1,1′-biphenyl]-4-carboxylate or tert-butyl 4′-{[(3R)-3-methyl-1-{[4-(propan-2-yl)phenyl]carbamoyl}-L-prolyl]amino}[1,1′-biphenyl]-4-carboxylate C59 (DIAST-1) (61.0 mg, 0.113 mmol) in 1,1,1,3,3,3-hexafluoropropan-2-ol (2 mL) was added methanesulfonic acid (7.31 μL, 0.113 mmol), whereupon the reaction mixture was stirred at 20° C. for 1 hour. It was then basified to a pH of approximately 7 by addition of ammonium hydroxide solution, and concentrated in vacuo. Purification via reversed-phase HPLC (Column: C18, 40×150 mm; Mobile phase A: water containing 0.05% ammonium hydroxide and 10 mM ammonium bicarbonate in water; Mobile phase B: acetonitrile; Gradient: 11% to 51% B; Flow rate: 60 mL/min) afforded 4′-{[(3S)-3-methyl-1-{[4-(propan-2-yl)phenyl]carbamoyl}-D-prolyl]amino}[1,1′-biphenyl]-4-carboxylic acid or 4′-{[(3R)-3-methyl-1-{[4-(propan-2-yl)phenyl]carbamoyl}-L-prolyl]amino}[1,1′-biphenyl]-4-carboxylic acid {34 [from C59 (DIAST-1)]} as a white solid. Yield: 25.0 mg, 51.5 μmol, 46%. LCMS m/z 486.3 [M+H]⁺. ¹H NMR (400 MHz, DMSO-d₆), characteristic peaks: δ 10.17 (s, 1H), 8.13 (s, 1H), 7.99 (d, J=8.4 Hz, 2H), 7.80-7.73 (m, 4H), 7.70 (d, half of AB quartet, J=8.8 Hz, 2H), 7.40 (d, J=8.6 Hz, 2H), 7.08 (d, J=8.6 Hz, 2H), 4.44 (d, J=8.2 Hz, 1H), 3.73 (t, J=8.9 Hz, 1H), 3.51-3.40 (m, 1H, assumed; partially obscured by water peak), 2.80 (septet, J=6.9 Hz, 1H), 2.10-1.99 (m, 1H), 1.91-1.77 (m, 1H), 1.16 (d, J=6.9 Hz, 6H), 1.00 (d, J=6.8 Hz, 3H).

### Example 35

### 4′-{[(3R)-3-Methyl-1-{[4-(propan-2-yl)phenyl]carbamoyl}-D-prolyl]amino}[1,1′-biphenyl]-4-carboxylic acid or 4′-{[(3S)-3-Methyl-1-{[4-(propan-2-yl)phenyl]carbamoyl}-L-prolyl]amino}[1,1′-biphenyl]-4-carboxylic acid {35 [from C60 (DIAST-2)]}

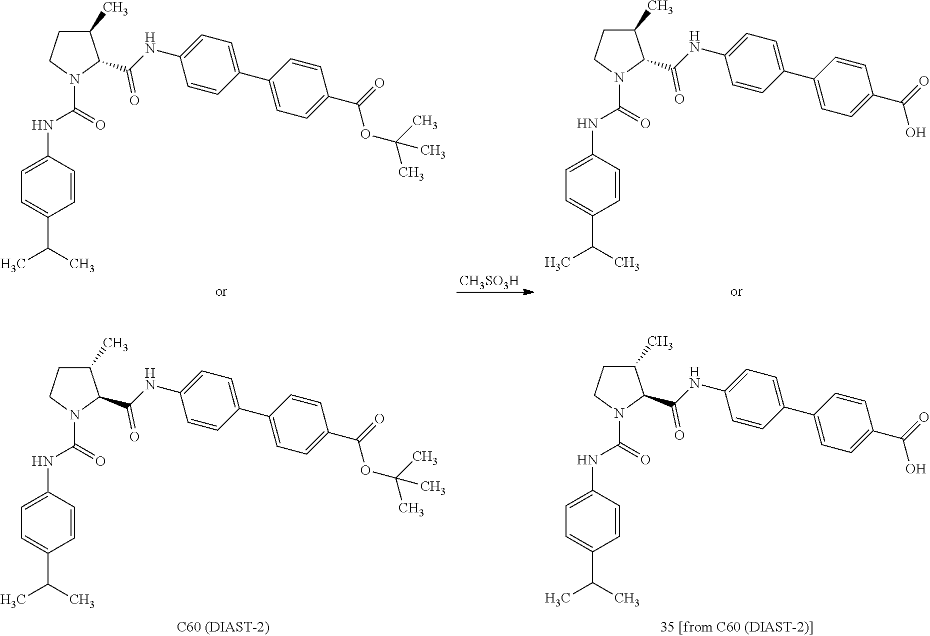 [SMILES: CC(C)c1ccc(NC(=O)N2CC[C@@H](C)[C@@H]2C(=O)Nc2ccc(-c3ccc(C(=O)OC(C)(C)C)cc3)cc2)cc1 ; "CH3SO3H" ; CC(C)c1ccc(NC(=O)N2CC[C@@H](C)[C@@H]2C(=O)Nc2ccc(-c3ccc(C(=O)O)cc3)cc2)cc1 ; CC(C)c1ccc(NC(=O)N2CC[C@H](C)[C@H]2C(=O)Nc2ccc(-c3ccc(C(=O)OC(C)(C)C)cc3)cc2)cc1 ; CC(C)c1ccc(NC(=O)N2CC[C@H](C)[C@H]2C(=O)Nc2ccc(-c3ccc(C(=O)O)cc3)cc2)cc1]

Methanesulfonic acid (9.11 μL, 0.140 mmol) was added to a solution of C60 (DIAST-2) (76.0 mg, 0.140 mmol) in 1,1,1,3,3,3-hexafluoropropan-2-ol (2 mL). After the reaction mixture had been stirred at 20° C. for 1 hour, it was basified to a pH of approximately 7 by addition of ammonium hydroxide solution, and concentrated in vacuo. Purification via reversed-phase HPLC (Column: C18, 40×150 mm; Mobile phase A: water containing 0.05% ammonium hydroxide and 10 mM ammonium bicarbonate in water; Mobile phase B: acetonitrile; Gradient: 11% to 51% B; Flow rate: 60 mL/min) provided 4′-{[(3R)-3-methyl-1-{[4-(propan-2-yl)phenyl]carbamoyl}-D-prolyl]amino}[1,1′-biphenyl]-4-carboxylic acid or 4′-{[(3S)-3-methyl-1-{[4-(propan-2-yl)phenyl]carbamoyl}-L-prolyl]amino}[1,1′-biphenyl]-4-carboxylic acid {35 [from C60 (DIAST-2)]} as a white solid. Yield: 38.8 mg, 79.9 μmol, 57%. LCMS m/z 486.3 [M+H]⁺. ¹H NMR (400 MHz, DMSO-d₆) δ 10.18 (s, 1H), 8.20 (s, 1H), 7.99 (d, J=8.5 Hz, 2H), 7.79-7.73 (m, 4H), 7.70 (d, half of AB quartet, J=8.9 Hz, 2H), 7.39 (d, J=8.6 Hz, 2H), 7.08 (d, J=8.6 Hz, 2H), 4.05 (d, J=5.2 Hz, 1H), 3.62 (t, J=6.8 Hz, 2H), 2.80 (septet, J=6.9 Hz, 1H), 2.35-2.24 (m, 1H), 2.21-2.11 (m, 1H), 1.69-1.58 (m, 1H), 1.18-1.12 (m, 9H).

### Example 36

### 4′-{[(4R)-4-Methoxy-1-{[4-(propan-2-yl)phenyl]carbamoyl}-D-prolyl]amino}[1,1′-biphenyl]-4-carboxylic acid (36)

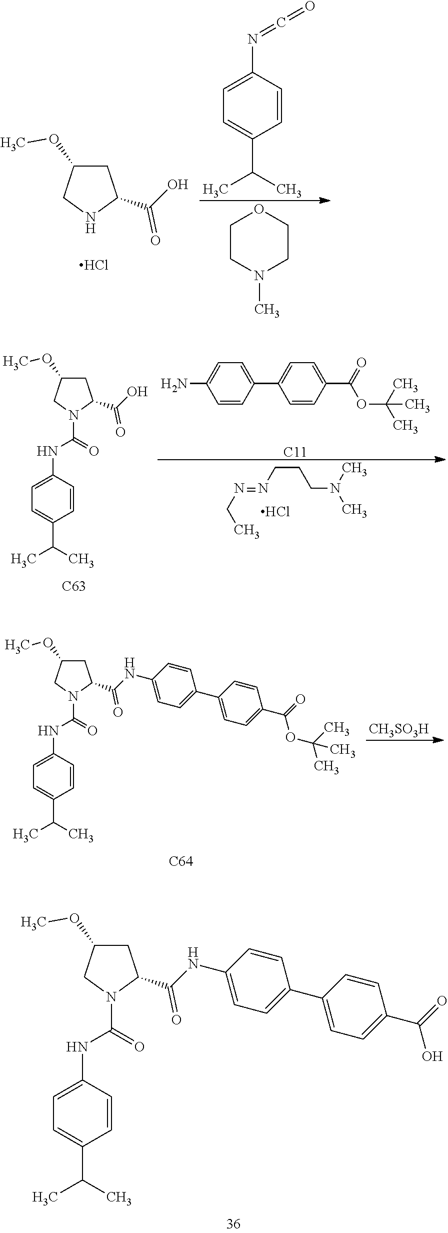

### Step 1. Synthesis of (4R)-4-methoxy-1-{[4-(propan-2-yl)phenyl]carbamoyl}-D-proline (C63)

4-Methylmorpholine (0.512 mL, 4.66 mmol) and 1-isocyanato-4-(propan-2-yl)benzene (250 mg, 1.55 mmol) were added to a 0° C. solution of (4R)-4-methoxy-D-proline, hydrochloride salt (282 mg, 1.55 mmol) in tetrahydrofuran (4.6 mL). The reaction mixture was stirred at 20° C. for 2 hours, whereupon LCMS analysis indicated conversion to C63: LCMS m/z 307.2 [M+H]⁺. Water (50 mL) was added to the reaction mixture, followed by sodium bicarbonate (195 mg, 2.32 mmol), resulting in a pH of 7 to 8. After the mixture had been washed with methyl tert-butyl ether (2×50 mL), the aqueous layer was acidified to a pH of approximately 3 by addition of concentrated hydrochloric acid, then extracted with ethyl acetate (2×30 mL). The combined ethyl acetate layers were washed with saturated aqueous sodium chloride solution (2×30 mL), dried over sodium sulfate, filtered, and concentrated in vacuo, affording C63 as a white solid. Yield: 400 mg, 1.31 mmol, 84%. ¹H NMR (400 MHz, DMSO-d₆) δ 12.35 (br s, 1H), 8.18 (s, 1H), 7.38 (d, J=8.5 Hz, 2H), 7.09 (d, J=8.6 Hz, 2H), 4.48-4.38 (m, 1H), 4.04-3.97 (m, 1H), 3.67 (dd, J=10.8, 5.5 Hz, 1H), 3.47 (dd, J=10.8, 3.0 Hz, 1H), 3.21 (s, 3H), 2.81 (septet, J=6.8 Hz, 1H), 2.31 (ddd, J=13.3, 9.1, 5.3 Hz, 1H), 2.14-2.05 (m, 1H), 1.17 (d, J=6.9 Hz, 6H).

### Step 2. Synthesis of tert-butyl 4′-{[(4R)-4-methoxy-1-{[4-(propan-2-yl)phenyl]carbamoyl}-D-prolyl]amino}[1,1′-biphenyl]-4-carboxylate (C64)

To a 25° C. solution of C63 (100 mg, 0.326 mmol) and C11 (87.9 mg, 0.326 mmol) in dichloromethane (2 mL) was added 1-[3-(dimethylamino)propyl]-3-ethylcarbodiimide hydrochloride (81.3 mg, 0.424 mmol). After the reaction mixture had been stirred at 25° C. for 2 hours, LCMS analysis indicated conversion to C64: LCMS m/z 558.4 [M+H]⁺. The reaction mixture was poured into water (30 mL) and extracted with dichloromethane (2×30 mL); the combined organic layers were washed with saturated aqueous sodium chloride solution (2×40 mL), dried over sodium sulfate, filtered, and concentrated in vacuo to provide C64 as a yellow gum. This material was used directly in the following step.

### Step 3. Synthesis of 4′-{[(4R)-4-methoxy-1-{[4-(propan-2-yl)phenyl]carbamoyl}-D-prolyl]amino}[1,1′-biphenyl]-4-carboxylic acid (36)

To a solution of C64 (from the previous step; 50.326 mmol) in 1,1,1,3,3,3-hexafluoropropan-2-ol (2 mL) was added methanesulfonic acid (84.8 μL, 1.31 mmol). After the reaction mixture had been stirred at 20° C. for 1 hour, it was diluted with water (30 mL) and extracted with ethyl acetate (2×30 mL). The combined organic layers were washed with saturated aqueous sodium chloride solution (2×30 mL), dried over sodium sulfate, filtered, concentrated in vacuo, and purified via reversed-phase HPLC (Column: C18, 40×150 mm; Mobile phase A: water containing 0.05% ammonium hydroxide and 10 mM ammonium bicarbonate in water; Mobile phase B: acetonitrile; Gradient: 5% to 45% B; Flow rate: 60 mL/min) to afford 4′-{[(4R)-4-methoxy-1-{[4-(propan-2-yl)phenyl]carbamoyl}-D-prolyl]amino}[1,1′-biphenyl]-4-carboxylic acid (36) as a white solid. Yield: 103 mg, 0.205 mmol, 63% over 2 steps. LCMS m/z 502.4 [M+H]⁺. ¹H NMR (400 MHz, DMSO-d₆) δ 9.91 (s, 1H), 8.28 (s, 1H), 7.99 (d, J=8.2 Hz, 2H), 7.77 (d, J=8.2 Hz, 2H), 7.71 (AB quartet, J_{AB}=9.2 Hz, Δv_{AB}=7.7 Hz, 4H), 7.41 (d, J=8.5 Hz, 2H), 7.10 (d, J=8.5 Hz, 2H), 4.48 (dd, J=8.8, 5.1 Hz, 1H), 4.13-4.04 (m, 1H), 3.80 (dd, J=10.3, 5.8 Hz, 1H), 3.57 (dd, J=10.3, 4.3 Hz, 1H), 3.25 (s, 3H), 2.80 (septet, J=6.9 Hz, 1H), 2.5-2.38 (m, 1H, assumed; partially obscured by solvent peak), 2.03 (dt, J=13.1, 5.0 Hz, 1H), 1.16 (d, J=6.9 Hz, 6H)

### Example 37

### 4′-{[1-({(1S)-1-[4-(Propan-2-yl)phenyl]ethyl}carbamoyl)-D-prolyl]amino}[1,1′-biphenyl]-4-carboxylic acid (37)

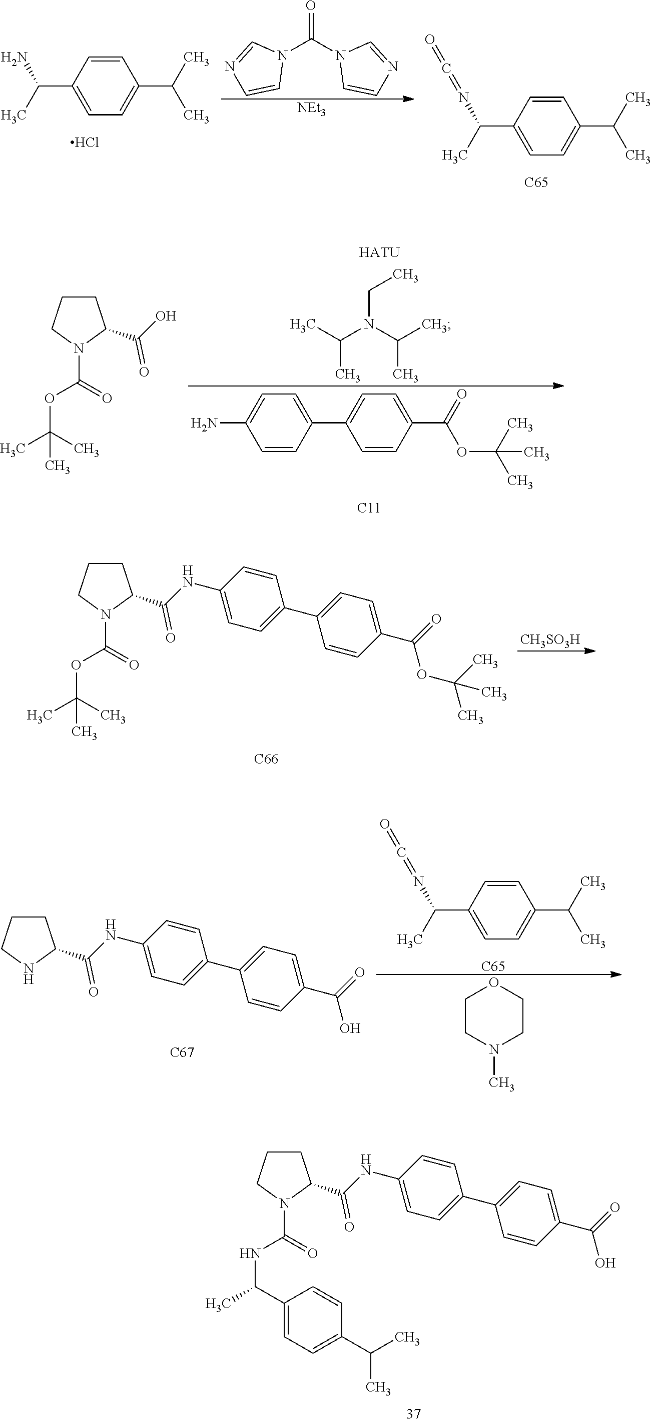

### Step 1. Synthesis of 1-[(1 S)-1-isocyanatoethyl]-4-(propan-2-yl)benzene (C65)

A mixture of (1S)-1-[4-(propan-2-yl)phenyl]ethan-1-amine, hydrochloride salt (100 mg, 0.501 mmol) and triethylamine (83.7 μL, 0.601 mmol) in acetonitrile (2.5 mL) was stirred for 10 minutes, whereupon 1,1′-carbonyldiimidazole (81.2 mg, 0.501 mmol) was added and the reaction mixture was stirred at 25° C. for 1 hour. Concentration in vacuo provided C65 as a colorless gum.

### Step 2. Synthesis of tert-butyl (2R)-2-{[4′-(tert-butoxycarbonyl)[1,1′-biphenyl]-4-yl]carbamoyl}pyrrolidine-1-carboxylate (C66)

To a solution of 1-(tert-butoxycarbonyl)-D-proline (1.50 g, 6.97 mmol) in N,N-dimethylformamide (35 mL) were added O-(7-azabenzotriazol-1-yl)-N,N,N′,N′-tetramethyluronium hexafluorophosphate (HATU; 5.30 g, 13.9 mmol) and N,N-diisopropylethylamine (2.43 mL, 14.0 mmol). After the reaction mixture had been stirred for 10 minutes at 25° C., C11 (1.88 g, 6.98 mmol) was added and stirring was continued at 25° C. for 3 days. The reaction mixture was then combined with a similar reaction carried out using 1-(tert-butoxycarbonyl)-D-proline (100 mg, 0.465 mmol) and poured into water (30 mL). Collection of the precipitate via filtration afforded C66 as a white solid. ¹H NMR analysis indicated that this material exists as a mixture of rotamers. Combined yield: 3.47 g, 7.44 mmol, 100%. LCMS m/z 489.2 [M+Na⁺]. ¹H NMR (400 MHz, DMSO-d₆), characteristic peaks: δ 10.14 (s, 1H), 7.95 (d, J=8.4 Hz, 2H), 7.78 (d, J=8.2 Hz, 2H), 7.76-7.68 (m, 4H), [4.31-4.25 (m) and 4.21 (dd, J=8.2, 4.4 Hz), total 1H], 3.48-3.39 (m, 1H, assumed; partially obscured by water peak), 2.29-2.12 (m, 1H), 1.97-1.73 (m, 3H), 1.56 (s, 9H), [1.40 (s) and 1.28 (s), total 9H].

### Step 3. Synthesis of 4′-(D-prolylamino)[1,1′-biphenyl]-4-carboxylic acid (C67)

To a solution of C66 (3.25 g, 6.97 mmol) in 1,1,1,3,3,3-hexafluoropropan-2-ol (34 mL) was added methanesulfonic acid (0.872 mL, 13.4 mmol), whereupon the reaction mixture was stirred at 20° C. for 16 hours. It was then concentrated in vacuo, and the residue was partitioned between ethyl acetate (40 mL) and water; solids were present in the organic layer, which were isolated via filtration to provide C67 as a white solid. Yield: 1.03 g, 3.32 mmol, 48%. LCMS m/z 311.1 [M+H]⁺. ¹H NMR (400 MHz, DMSO-d₆), characteristic peaks: δ 8.71 (br s, 1H), 8.00 (d, J=8.5 Hz, 2H), 7.80 (d, J=8.5 Hz, 2H), 7.77 (s, 4H), 4.48-4.35 (m, 1H), 2.49-2.36 (m, 1H), 2.07-1.88 (m, 3H).

### Step 4. Synthesis of 4′-{[1-({(1 S)-1-[4-(propan-2-yl)phenyl]ethyl}carbamoyl)-D-prolyl]amino}[1,1′-biphenyl]-4-carboxylic acid (37)

4-Methylmorpholine (0.162 mL, 1.47 mmol) was added to a suspension of C67 (100 mg, 0.246 mmol) in tetrahydrofuran (1 mL). After the mixture had been stirred for 5 minutes, C65 (50 mg, 0.26 mmol) was added and stirring was continued at 25° C. for 1 hour, whereupon water (5 mL) was added to the reaction mixture and the pH was adjusted to approximately 6. The mixture was extracted with ethyl acetate (3×5 mL), and the combined organic layers were washed with saturated aqueous sodium chloride solution (10 mL), dried over sodium sulfate, filtered, and concentrated in vacuo. Purification via reversed-phase HPLC (Column: C18, 40×150 mm; Mobile phase A: water containing 0.05% ammonium hydroxide and 10 mM ammonium bicarbonate in water; Mobile phase B: acetonitrile; Gradient: 11% to 51% B; Flow rate: 60 mL/min) afforded 4′-{[1-({(1S)-1-[4-(propan-2-yl)phenyl]ethyl}carbamoyl)-D-prolyl]amino}[1,1′-biphenyl]-4-carboxylic acid (37) as a white solid. Yield: 38.1 mg, 76.3 μmol, 31%. LCMS m/z 500.4 [M+H]⁺. ¹H NMR (400 MHz, DMSO-d₆), characteristic peaks: δ 10.07 (s, 1H), 7.98 (d, J=8.4 Hz, 2H), 7.75 (d, J=8.4 Hz, 2H), 7.71-7.63 (m, 4H), 7.21 (AB quartet, J_{AB}=8.1 Hz, Δv_{AB}=43.1 Hz, 4H), 6.50 (d, J=8.3 Hz, 1H), 4.86-4.76 (m, 1H), 4.44-4.37 (m, 1H), 3.56-3.47 (m, 1H), 2.90-2.78 (m, 1H), 2.15-2.03 (m, 1H), 2.03-1.86 (m, 3H), 1.36 (d, J=7.0 Hz, 3H), 1.17 (d, J=6.9 Hz, 6H).

### Example 38

### 3-[6-({1-[(4-Cyclobutylphenyl)carbamoyl]-D-prolyl}amino)pyridin-3-yl]benzoic acid (38)

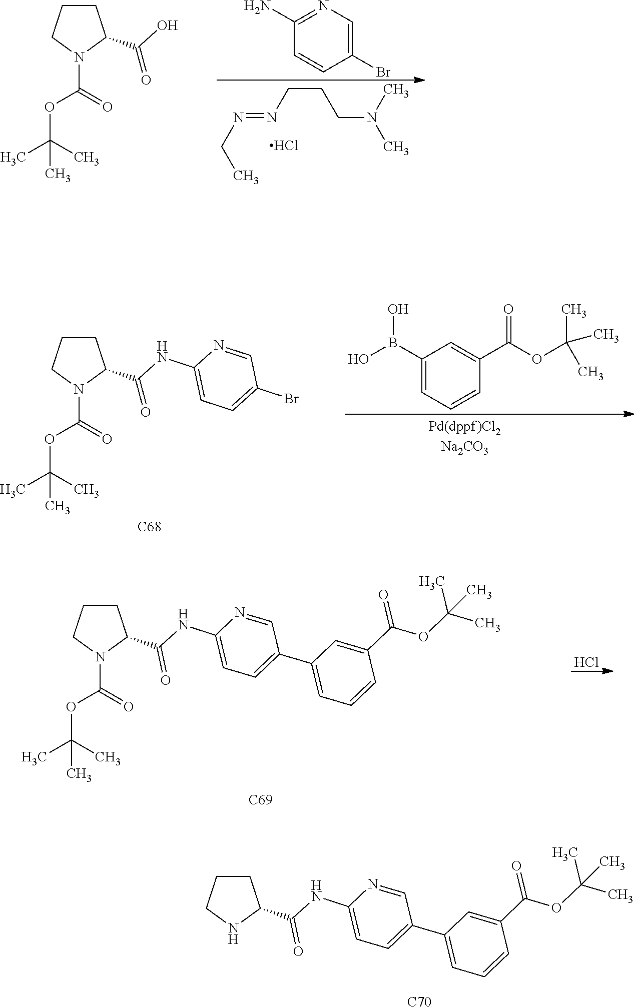

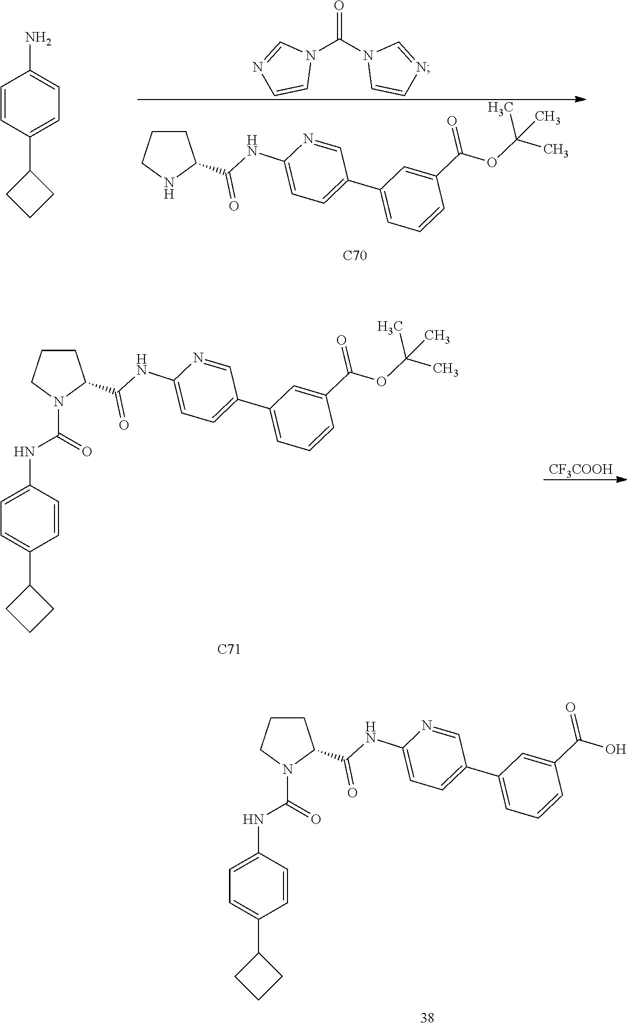

### Step 1. Synthesis of tert-butyl (2R)-2-[(5-bromopyridin-2-yl)carbamoyl]pyrrolidine-1-carboxylate (C68)

To a 0° C. solution of 1-(tert-butoxycarbonyl)-D-proline (70.0 g, 325 mmol) and 5-bromopyridin-2-amine (50.6 g, 292 mmol) in dichloromethane (1.2 L) was added 1-[3-(dimethylamino)propyl]-3-ethylcarbodiimide hydrochloride (81.0 g 423 mmol), whereupon the reaction mixture was heated to 20° C. and allowed to stir at 20° C. for 40 hours. It was then concentrated under reduced pressure, poured into water (800 mL), and extracted with dichloromethane (3×600 mL); the combined organic layers were washed sequentially with aqueous sodium bicarbonate solution (2×400 mL), water (5×700 mL), and saturated aqueous sodium chloride solution (600 mL), dried over sodium sulfate, filtered, and concentrated in vacuo. The residue was stirred for 1 hour in methyl tert-butyl ether (80 mL), whereupon it was filtered; the filter cake was washed with methyl tert-butyl ether (3×20 mL) to afford C68 (60.1 g). This material was combined with the product of a similar reaction carried out using 5-bromopyridin-2-amine (8.04 g, 46.5 mmol) to provide C68 as a white solid. By ¹H NMR analysis, this material exists as a mixture of rotamers at room temperature. Combined yield: 67.7 g, 183 mmol, 54%. LCMS m/z 372.0 (bromine isotope pattern observed) [M+H]⁺. ¹H NMR (400 MHz, DMSO-d₆) δ [10.76 (s) and 10.70 (s), total 1H], 8.44 (d, J=2.4 Hz, 1H), 8.12-7.96 (m, 2H), [4.40 (dd, J=8.6, 3.0 Hz) and 4.35 (dd, J=8.3, 4.3 Hz), total 1H], 3.45-3.28 (m, 2H), 2.25-2.08 (m, 1H), 1.94-1.71 (m, 3H), [1.39 (s) and 1.24 (s), total 9H].

### Step 2. Synthesis of tert-butyl (2R)-2-({5-[3-(tert-butoxycarbonyl)phenyl]pyridin-2-yl}carbamoyl)pyrrolidine-1-carboxylate (C69)

A mixture of C68 (500 mg, 1.35 mmol), [3-(tert-butoxycarbonyl)phenyl]boronic acid (360 mg, 1.62 mmol), sodium carbonate (286 mg, 2.70 mmol), and [1,1′-bis(diphenylphosphino)ferrocene]dichloropalladium(II) (98.8 mg, 0.135 mmol) in a mixture of 1,4-dioxane (6.8 mL), and water (1.0 mL) was sparged 3 times with nitrogen. After the reaction mixture had been heated at 80° C. overnight, it was allowed to cool to room temperature before being filtered through a plug of diatomaceous earth. The filtrate was concentrated under reduced pressure, and the residue was partitioned between ethyl acetate and water; the aqueous layer was extracted twice with ethyl acetate, and the combined organic layers were washed with saturated aqueous sodium chloride solution, dried over magnesium sulfate, filtered, and concentrated in vacuo. Silica gel chromatography (Gradient: 0% to 100% ethyl acetate in heptane) afforded C69 as a pale-yellow foam; by ¹H NMR analysis, this material exists as a mixture of rotamers at room temperature. Yield: 609 mg, 1.30 mmol, 96%. LCMS m/z 468.4 [M+H]⁺. ¹H NMR (600 MHz, DMSO-d₆) δ [10.73 (s) and 10.67 (s), total 1H], 8.70-8.66 (m, 1H), 8.24-8.12 (m, 3H), 7.97 (br d, J=7.8 Hz, 1H), 7.91 (d, J=7.8 Hz, 1H), 7.61 (t, J=7.7 Hz, 1H), [4.48-4.42 (m) and 4.42-4.37 (m), total 1H], 3.45-3.39 (m, 1H), 3.39-3.33 (m, 1H), 2.27-2.12 (m, 1H), 1.94-1.85 (m, 2H), 1.85-1.74 (m, 1H), 1.58 (s, 9H), [1.40 (s) and 1.27 (s), total 9H].

### Step 3. Synthesis of tert-butyl 3-[6-(D-prolylamino)pyridin-3-yl]benzoate (C70)

A solution of hydrogen chloride in 1,4-dioxane (4 M; 2.5 mL, 10 mmol) was added drop-wise to a solution of C69 (424 mg, 0.907 mmol) in ethyl acetate (2.5 mL). After the reaction mixture had been stirred for 80 minutes, the precipitate was collected via filtration to provide a white solid (350 mg). A portion of this material (252 mg) was dissolved in dichloromethane, washed with saturated aqueous sodium bicarbonate solution, dried over magnesium sulfate, filtered, and concentrated in vacuo to provide C70 as a colorless oil (165 mg). Yield: 165 mg, 0.449 mmol, 69% (yield adjusted to account for only part of the crude product being neutralized). LCMS m/z 368.4 [M+H]⁺. ¹H NMR (600 MHz, DMSO-d₆) δ 10.41 (s, 1H), 8.69-8.64 (m, 1H), 8.20 (AB quartet, upfield doublet is broadened, J_{AB}=8.6 Hz, Δv_{AB}=31.7 Hz, 2H), 8.15-8.13 (m, 1H), 7.96 (br d, J=7.8 Hz, 1H), 7.91 (br d, J=7.8 Hz, 1H), 7.61 (t, J=7.7 Hz, 1H), 3.79 (dd, J=8.6, 5.6 Hz, 1H), 2.99-2.92 (m, 1H), 2.89-2.83 (m, 1H), 2.13-2.04 (m, 1H), 1.86-1.78 (m, 1H), 1.70-1.63 (m, 2H), 1.57 (s, 9H).

### Step 4. Synthesis of tert-butyl 3-[6-({1-[(4-cyclobutylphenyl)carbamoyl]-D-prolyl}amino)pyridin-3-yl]benzoate (C71)

To a 0° C. solution of 1,1′-carbonyldiimidazole (21.3 mg, 0.131 mmol) in acetonitrile (0.24 mL) was added 4-cyclobutylaniline (19.3 mg, 0.131 mmol). The reaction mixture was stirred at 0° C. for 40 minutes, whereupon C70 (43.8 mg, 0.119 mmol) was added, and stirring was continued for 2.25 hours. After removal of solvent via concentration under reduced pressure, the residue was partitioned between water and ethyl acetate. The aqueous layer was extracted with ethyl acetate, and the combined organic layers were washed with saturated aqueous sodium chloride solution and concentrated in vacuo, providing C71 as a light-orange solid. Yield: 49.5 mg, 91.6 μmol, 77%. LCMS m/z 541.6 [M+H]⁺. ¹H NMR (600 MHz, DMSO-d₆), characteristic peaks: δ 10.64 (s, 1H), 8.68 (d, J=2.6 Hz, 1H), 8.23 (s, 1H), 8.20-8.12 (m, 3H), 7.96 (br d, J=8.0 Hz, 1H), 7.90 (br d, J=7.7 Hz, 1H), 7.61 (t, J=7.8 Hz, 1H), 7.41 (d, J=8.6 Hz, 2H), 7.09 (d, J=8.6 Hz, 2H), 4.65-4.60 (m, 1H), 3.67-3.61 (m, 1H), 3.55-3.48 (m, 1H), 3.47-3.39 (m, 1H), 1.57 (s, 9H).

### Step 5. Synthesis of 3-[6-({1-[(4-cyclobutylphenyl)carbamoyl]-D-prolyl}amino)pyridin-3-yl]benzoic acid (38)

A solution of C71 (49.0 mg, 90.6 μmol) in dichloromethane (0.45 mL) was treated drop-wise with trifluoroacetic acid (0.15 mL), whereupon the reaction mixture was stirred for 1 hour and 40 minutes. After concentration in vacuo, the residue was purified via reversed-phase HPLC (Column: Waters Sunfire C18, 19×100 mm, 5 μm; Mobile phase A: 0.05% trifluoroacetic acid in water (v/v); Mobile phase B: 0.05% trifluoroacetic acid in acetonitrile (v/v); Gradient: 5% to 95% B over 8.54 minutes, then 95% B for 1.46 minutes; Flow rate: 25 mL/minute), affording 3-[6-({1-[(4-cyclobutylphenyl)carbamoyl]-D-prolyl}amino)pyridin-3-yl]benzoic acid (38). Yield: 39.9 mg, 82.3 μmol, 91%. LCMS m/z 485.5 [M+H]⁺. ¹H NMR (600 MHz, DMSO-d₆), characteristic peaks: δ 10.64 (s, 1H), 8.70-8.65 (m, 1H), 8.24 (s, 1H), 8.19 (br s, 1H), 8.17 (d, half of AB quartet, J=8.9 Hz, 1H), 8.14 (dd, component of ABX system, J=8.8, 2.4 Hz, 1H), 7.99-7.92 (m, 2H), 7.61 (t, J=7.8 Hz, 1H), 7.43-7.38 (m, 2H), 7.08 (d, J=8.5 Hz, 2H), 4.64-4.59 (m, 1H), 3.67-3.60 (m, 1H, assumed; partially obscured by water peak), 2.27-2.20 (m, 2H), 2.20-2.14 (m, 1H), 2.07-1.89 (m, 6H), 1.81-1.74 (m, 1H).

Examples 8-15 and 39-229 were prepared using similar methods described herein with suitable starting materials, reactants, and reagents. Table 1 includes their structures and physicochemical data

TABLE 1
Structure and physicochemical data for Examples 8-15 and 39-229.

| | | ¹H NMR (600 MHz, DMSO-d₆) δ; Mass spectrum, observed |
|---|---|---|
| | | ion m/z [M + H]⁺ or HPLC retention time; Mass spectrum |
| Example | Structure | m/z [M + H]⁺ (unless otherwise indicated) |
| 8 | 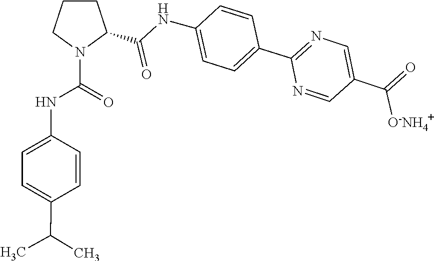 | ¹H NMR, characteristic peaks: δ 10.31 (s, 1H), 9.22 (s, 2H), 8.40 (d, J = 8.8 Hz, 2H), 8.21 (s, 1H), 7.80 (d, J = 8.7 Hz, 2H), 7.39 (d, J = 8.3 Hz, 2H), 7.08 (d, J = 8.6 Hz, 2H), 4.48 (dd, J = 8.3, 3.8 Hz, 1H), 3.68- 3.61 (m, 1H, assumed; partially obscured by water peak), 2.79 (septet, J = 7.0 Hz, 1H), 2.24-2.15 (m, 1H), 2.08-2.00 (m, 1H), 2.00-1.90 (m, 2H), 1.15 (d, J = 6.9 Hz, 6H); 474.4 |
| 9 | 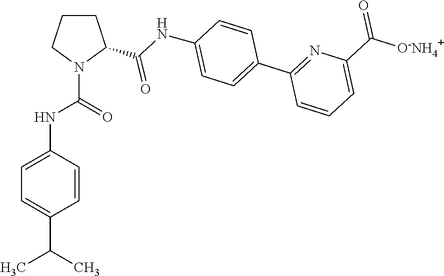 | ¹H NMR, characteristic peaks: δ 10.19 (s, 1H), 8.20 (s, 1H), 8.18-8.13 (m, 3H), 8.02 (t, J = 7.8 Hz, 1H), 7.94 (dd, component of ABX system, J = 7.6, 0.9 Hz, 1H), 7.76 (d, J = 8.1 Hz, 2H), 7.41- 7.37 (m, 2H), 7.08 (d, J = 8.5 Hz, 2H), 4.47 (dd, J = 8.3, 3.7 Hz, 1H), 3.68-3.61 (m, 1H, assumed; partially obscured by water peak), 2.79 (septet, J = 7.0 Hz, 1H), 2.24-2.14 (m, 1H), 2.08-1.99 (m, 1H), 1.99-1.89 (m, 2H), 1.16 (d, J = 6.9 Hz, 6H); 473.4 |
| 10 | 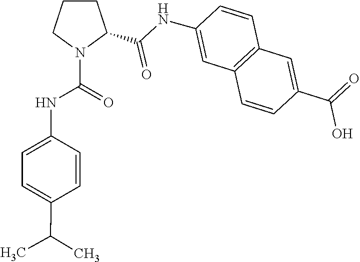 [SMILES: CC(C)c1ccc(NC(=O)N2CCC[C@@H]2C(=O)Nc2ccc3cc(C(=O)O)ccc3c2)cc1] | 12.93 (br s, 1H), 10.34 (s, 1H), 8.50 (br s, 1H), 8.39 (br s, 1H), 8.22 (s, 1H), 8.05 (d, J = 8.9 Hz, 1H), 7.91 (dd, component of ABX system, J = 8.5, 1.7 Hz, 1H), 7.86 (d, half of AB quartet, J = 8.7 Hz, 1H), 7.68 (dd, J = 8.9, 2.1 Hz, 1H), 7.40 (d, J = 8.5 Hz, 2H), 7.08 (d, J = 8.6 Hz, 2H), 4.52 (dd, J = 8.4, 3.6 Hz, 1H), 3.69-3.63 (m, 1H), 3.56-3.50 (m, 1H), 2.80 (septet, J = 6.9 Hz, 1H), 2.26-2.17 (m, 1H), 2.10- 2.01 (m, 1H), 2.01-1.92 (m, 2H), 1.15 (d, J = 6.9 Hz, 6H); 446.4 |
| 11 | 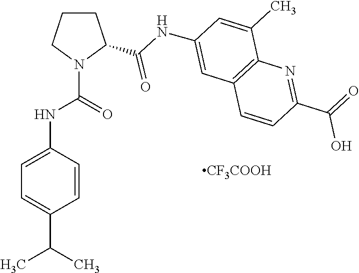 [SMILES: Cc1cc(NC(=O)[C@H]2CCCN2C(=O)Nc2ccc(C(C)C)cc2)cc2ccc(C(=O)O)nc12 ; "·CF3COOH"] | 10.35 (s, 1H), 8.37 (d, J = 8.5 Hz, 1H), 8.28 (d, J = 2.3 Hz, 1H), 8.22 (s, 1H), 8.04 (d, J = 8.5 Hz, 1H), 7.80-7.77 (m, 1H), 7.40 (d, J = 8.6 Hz, 2H), 7.08 (d, J = 8.7 Hz, 2H), 4.51 (dd, J = 8.4, 3.7 Hz, 1H), 3.69-3.63 (m, 1H), 3.56-3.50 (m, 1H), 2.80 (septet, J = 6.9 Hz, 1H), 2.75 (s, 3H), 2.25-2.17 (m, 1H), 2.09-2.00 (m, 1H), 2.00-1.92 (m, 2H), 1.15 (d, J = 6.9 Hz, 6H); 461.4 |
| 12 | 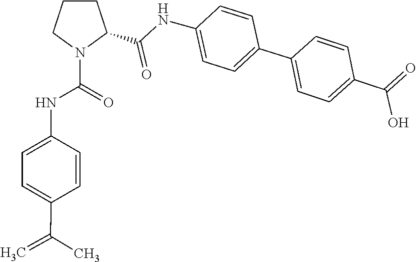 [SMILES: C=C(C)c1ccc(NC(=O)N2CCC[C@@H]2C(=O)Nc2ccc(-c3ccc(C(=O)O)cc3)cc2)cc1] | ¹H NMR (400 MHz, methanol-d₄) δ 8.07 (d, J = 8.4 Hz, 2H), 7.75-7.69 (m, 4H), 7.66 (d, half of AB quartet, J = 8.7 Hz, 2H), 7.40 (s, 4H), 5.31 (br s, 1H), 5.01- 4.97 (m, 1H), 4.59 (dd, J = 8.3, 3.5 Hz, 1H), 3.80-3.71 (m, 1H), 3.65-3.56 (m, 1H), 2.39-2.26 (m, 1H), 2.25-2.03 (m, 3H), 2.11 (br s, 3H); 470.3 |
| 13 | 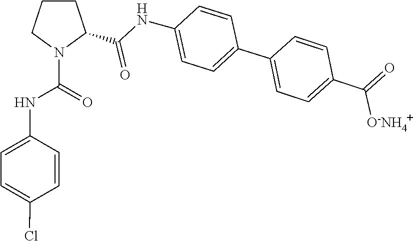 | 10.15 (s, 1H), 8.44 (s, 1H), 7.99 (d, J = 8.4 Hz, 2H), 7.78 (d, J = 8.4 Hz, 2H), 7.72 (AB quartet, J_{AB} = 8.8 Hz, Δv_{AB} = 15.0 Hz, 4H), 7.55 (d, J = 8.8 Hz, 2H), 7.27 (d, J = 8.9 Hz, 2H), 4.47 (dd, J = 8.3, 3.8 Hz, 1H), 3.68-3.62 (m, 1H), 3.56-3.50 (m, 1H), 2.24-2.16 (m, 1H), 2.07-1.99 (m, 1H), 1.99-1.89 (m, 2H); 464.4 (chlorine isotope pattern observed) |
| 14 | 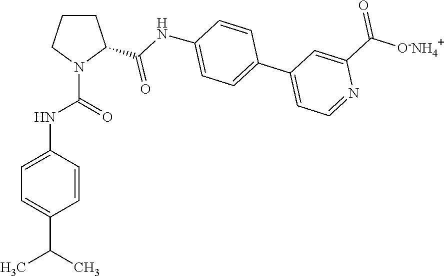 | 10.22 (s, 1H), 8.71 (d, J = 5.1 Hz, 1H), 8.28 (d, J = 1.9 Hz, 1H), 8.20 (s, 1H), 7.93 (dd, J = 5.2, 1.9 Hz, 1H), 7.82 (AB quartet, J_{AB} = 8.8 Hz, Δv_{AB} = 39.5 Hz, 4H), 7.39 (d, J = 8.5 Hz, 2H), 7.08 (d, J = 8.6 Hz, 2H), 4.47 (dd, J = 8.3, 3.8 Hz, 1H), 3.65 (ddd, J = 9.4, 7.7, 4.7 Hz, 1H), 3.54-3.48 (m, 1H, assumed; partially obscured by water peak), 2.80 (septet, J = 6.9 Hz, 1H), 2.24- 2.15 (m, 1H), 2.08-1.99 (m, 1H), 1.99- 1.90 (m, 2H), 1.16 (d, J = 6.9 Hz, 6H); 473.5 |
| 15 | 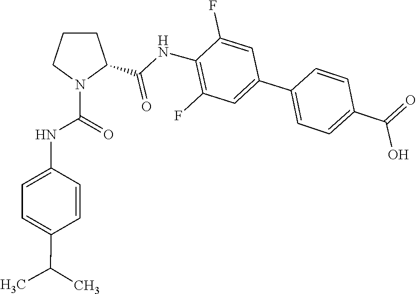 [SMILES: CC(C)c1ccc(NC(=O)N2CCC[C@@H]2C(=O)Nc2c(F)cc(-c3ccc(C(=O)O)cc3)cc2F)cc1] | 13.06 (br s, 1H), 9.76 (s, 1H), 8.18 (s, 1H), 7.95 (AB quartet, J_{AB} = 8.4 Hz, Δv_{AB} = 74.9 Hz, 4H), 7.60 (d, J = 9.0 Hz, 2H), 7.42 (d, J = 8.6 Hz, 2H), 7.10 (d, J = 8.5 Hz, 2H), 4.57 (dd, J = 8.6, 3.0 Hz, 1H), 3.70-3.63 (m, 1H), 3.51-3.45 (m, 1H), 2.81 (septet, J = 7.0 Hz, 1H), 2.26-2.17 (m, 1H), 2.04-1.92 (m, 3H), 1.17 (d, J = 6.9 Hz, 6H); 508.4 |
| 39 | 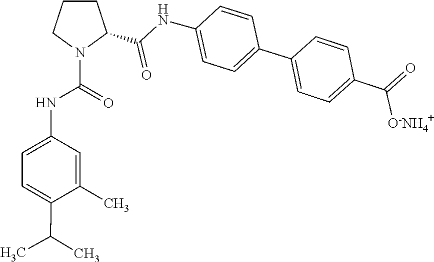 | 10.13 (s, 1H), 8.11 (s, 1H), 7.98 (d, J = 8.4 Hz, 2H), 7.77 (d, J = 8.5 Hz, 2H), 7.72 (AB quartet, J_{AB} = 8.8 Hz, Δv_{AB} = 17.9 Hz, 4H), 7.29-7.25 (m, 2H), 7.06 (d, J = 8.2 Hz, 1H), 4.46 (dd, J = 8.3, 3.7 Hz, 1H), 3.67-3.60 (m, 1H), 3.53-3.47 (m, 1H), 3.01 (septet, J = 6.8 Hz, 1H), 2.22 (s, 3H), 2.21-2.15 (m, 1H), 2.07-1.99 (m, 1H), 1.99-1.89 (m, 2H), 1.13 (d, J = 6.8 Hz, 6H); 486.4 |
| 40 | 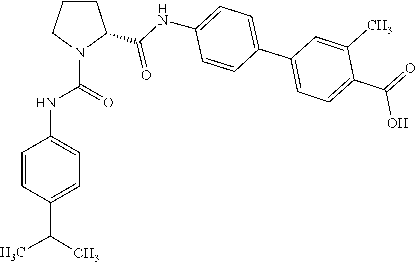 [SMILES: Cc1cc(-c2ccc(NC(=O)[C@H]3CCCN3C(=O)Nc3ccc(C(C)C)cc3)cc2)ccc1C(=O)O] | ¹H NMR, characteristic peaks: δ 10.12 (s, 1H), 8.19 (s, 1H), 7.89 (d, J = 8.2 Hz, 1H), 7.70 (AB quartet, J_{AB} = 8.9 Hz, Δv_{AB} = 14.7 Hz, 4H), 7.60 (br s, 1H), 7.56 (dd, J = 8.1, 2.0 Hz, 1H), 7.39 (d, J = 8.5 Hz, 2H), 7.08 (d, J = 8.6 Hz, 2H), 4.46 (dd, J = 8.3, 3.7 Hz, 1H), 3.67-3.61 (m, 1H), 2.80 (septet, J = 7.0 Hz, 1H), 2.58 (s, 3H), 2.23-2.15 (m, 1H), 2.07-1.99 (m, 1H), 1.99-1.89 (m, 2H), 1.16 (d, J = 6.9 Hz, 6H); 486.4 |
| 41 | 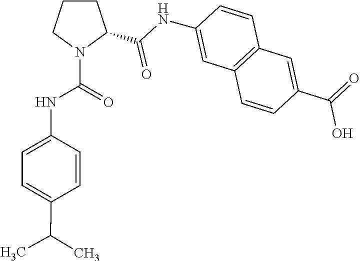 [SMILES: CC(C)c1ccc(NC(=O)N2CCC[C@@H]2C(=O)Nc2ccc3cc(C(=O)O)ccc3c2)cc1] | 10.35 (s, 1H), 8.50 (br s, 1H), 8.39 (br s, 1H), 8.22 (s, 1H), 8.05 (d, J = 8.9 Hz, 1H), 7.91 (dd, component of ABX system, J = 8.6, 1.7 Hz, 1H), 7.86 (d, half of AB quartet, J = 8.6 Hz, 1H), 7.68 (dd, J = 9.0, 2.1 Hz, 1H), 7.39 (d, J = 8.7 Hz, 2H), 7.08 (d, J = 8.6 Hz, 2H), 4.51 (dd, J = 8.4, 3.7 Hz, 1H), 3.69-3.62 (m, 1H), 3.56-3.50 (m, 1H, assumed; partially obscured by water peak), 2.79 (septet, J = 6.9 Hz, 1H), 2.25-2.17 (m, 1H), 2.09-2.01 (m, 1H), 2.01-1.92 (m, 2H), 1.15 (d, J = 6.9 Hz, 6H); 446.4 |
| 42 | 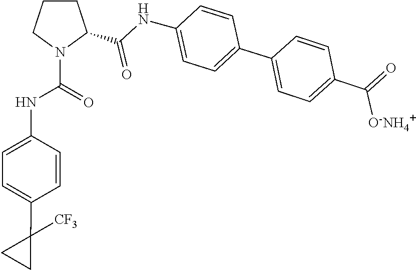 | 10.15 (s, 1H), 8.39 (s, 1H), 7.98 (d, J = 8.5 Hz, 2H), 7.77 (d, J = 8.5 Hz, 2H), 7.72 (AB quartet, J_{AB} = 8.9 Hz, Δv_{AB} = 16.2 Hz, 4H), 7.51 (d, J = 8.5 Hz, 2H), 7.30 (d, J = 8.6 Hz, 2H), 4.48 (dd, J = 8.3, 3.8 Hz, 1H), 3.69-3.63 (m, 1H), 3.56-3.50 (m, 1H), 2.24-2.16 (m, 1H), 2.08-1.99 (m, 1H), 1.99-1.90 (m, 2H), 1.29-1.24 (m, 2H), 1.07-1.01 (m, 2H); 538.4 |
| 43 | 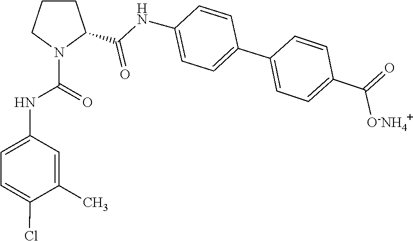 | 10.15 (s, 1H), 8.35 (s, 1H), 7.99 (d, J = 8.4 Hz, 2H), 7.78 (d, J = 8.5 Hz, 2H), 7.72 (AB quartet, J_{AB} = 8.9 Hz, Δv_{AB} = 15.3 Hz, 4H), 7.53 (br s, 1H), 7.38 (dd, component of ABX system, J = 8.8, 2.6 Hz, 1H), 7.23 (d, half of AB quartet, J = 8.8 Hz, 1H), 4.46 (dd, J = 8.3, 3.8 Hz, 1H), 3.67-3.61 (m, 1H), 3.55-3.49 (m, 1H), 2.25 (s, 3H), 2.22-2.16 (m, 1H), 2.07-1.99 (m, 1H), 1.99-1.89 (m, 2H); 478.4 (chlorine isotope pattern observed) |
| 44 | 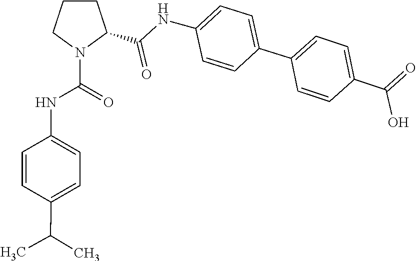 [SMILES: CC(C)c1ccc(NC(=O)N2CCC[C@@H]2C(=O)Nc2ccc(-c3ccc(C(=O)O)cc3)cc2)cc1] | ¹H NMR (400 MHz, DMSO-d₆) δ 12.90 (br s, 1H), 10.13 (s, 1H), 8.19 (s, 1H), 7.99 (d, J = 8.5 Hz, 2H), 7.78 (d, J = 8.5 Hz, 2H), 7.72 (AB quartet, J_{AB} = 9.0 Hz, Δv_{AB} = 12.3 Hz, 4H), 7.40 (d, J = 8.6 Hz, 2H), 7.09 (d, J = 8.6 Hz, 2H), 4.47 (dd, J = 8.3, 3.6 Hz, 1H), 3.70-3.60 (m, 1H), 3.56-3.47 (m, 1H), 2.80 (septet, J = 6.9 Hz, 1H), 2.25-2.13 (m, 1H), 2.10-1.88 (m, 3H), 1.16 (d, J = 6.9 Hz, 6H); 472.4 |
| 45 | 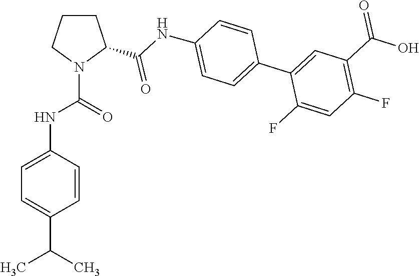 [SMILES: CC(C)c1ccc(NC(=O)N2CCC[C@@H]2C(=O)Nc2ccc(-c3cc(C(=O)O)c(F)cc3F)cc2)cc1] | 3.06 minutes¹; 508.4 |
| 46 | 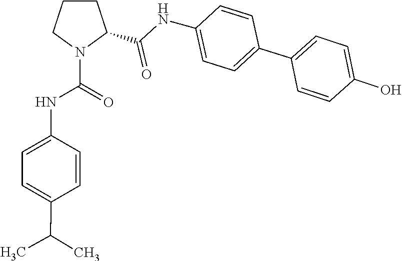 [SMILES: CC(C)c1ccc(NC(=O)N2CCC[C@@H]2C(=O)Nc2ccc(-c3ccc(O)cc3)cc2)cc1] | 2.98 minutes¹; 444.4 |
| 47 | 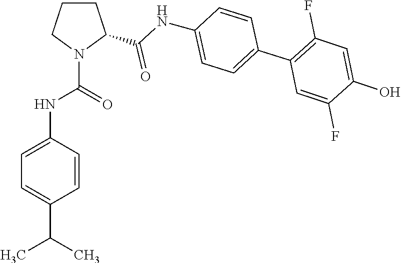 [SMILES: CC(C)c1ccc(NC(=O)N2CCC[C@@H]2C(=O)Nc2ccc(-c3cc(F)c(O)cc3F)cc2)cc1] | 3.12 minutes¹; 480.4 |
| 48 | 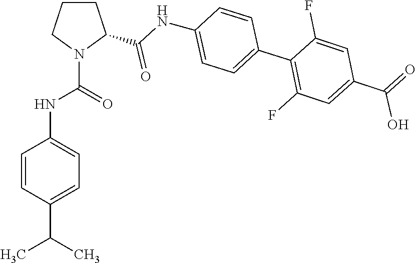 [SMILES: CC(C)c1ccc(NC(=O)N2CCC[C@@H]2C(=O)Nc2ccc(-c3c(F)cc(C(=O)O)cc3F)cc2)cc1] | 3.09 minutes¹; 508.4 |
| 49 | 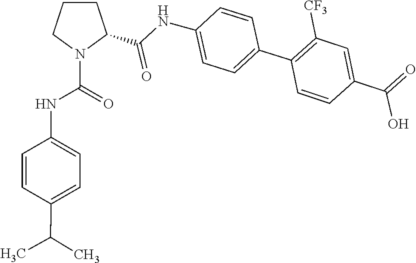 [SMILES: CC(C)c1ccc(NC(=O)N2CCC[C@@H]2C(=O)Nc2ccc(-c3ccc(C(=O)O)cc3C(F)(F)F)cc2)cc1] | 3.17 minutes¹; 540.4 |
| 50 | 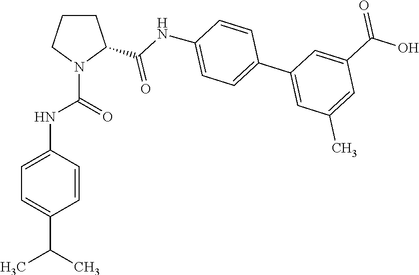 [SMILES: Cc1cc(C(=O)O)cc(-c2ccc(NC(=O)[C@H]3CCCN3C(=O)Nc3ccc(C(C)C)cc3)cc2)c1] | 3.12 minutes¹; 486.4 |
| 51 | 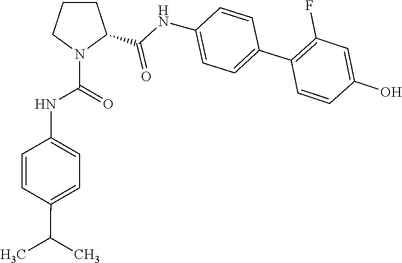 [SMILES: CC(C)c1ccc(NC(=O)N2CCC[C@@H]2C(=O)Nc2ccc(-c3ccc(O)cc3F)cc2)cc1] | 3.06 minutes¹; 462.4 |
| 52 | 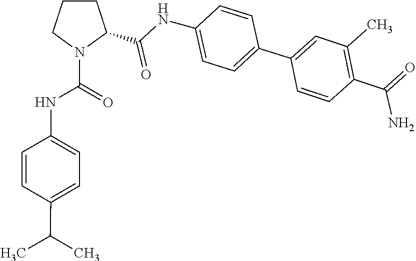 [SMILES: Cc1cc(-c2ccc(NC(=O)[C@H]3CCCN3C(=O)Nc3ccc(C(C)C)cc3)cc2)ccc1C(N)=O] | 2.82 minutes¹; 485.5 |
| 53 | 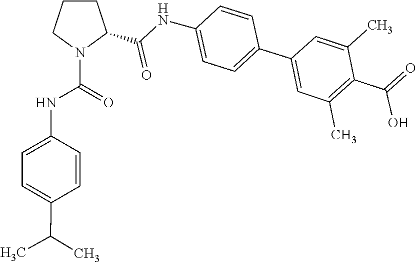 [SMILES: Cc1cc(-c2ccc(NC(=O)[C@H]3CCCN3C(=O)Nc3ccc(C(C)C)cc3)cc2)cc(C)c1C(=O)O] | 3.06 minutes¹; 500.5 |
| 54 | 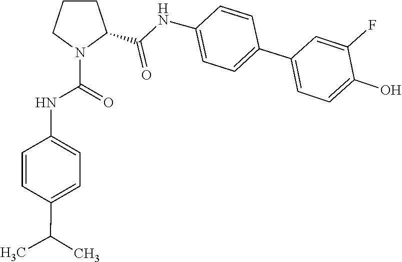 [SMILES: CC(C)c1ccc(NC(=O)N2CCC[C@@H]2C(=O)Nc2ccc(-c3ccc(O)c(F)c3)cc2)cc1] | 3.05 minutes¹; 462.4 |
| 55 | 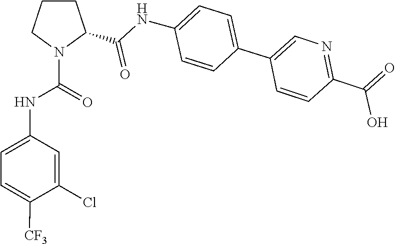 [SMILES: O=C(O)c1ccc(-c2ccc(NC(=O)[C@H]3CCCN3C(=O)Nc3ccc(C(F)(F)F)c(Cl)c3)cc2)cn1] | 10.24 (s, 1H), 9.01 (br d, J = 2.3 Hz, 1H), 8.89 (s, 1H), 8.24 (dd, J = 8.2, 2.4 Hz, 1H), 8.09 (dd, J = 8.2, 0.8 Hz, 1H), 7.97 (br s, 1H), 7.79 (AB quartet, J_{AB} = 8.9 Hz, Δv_{AB} = 14.3 Hz, 4H), 7.69 (AB quartet, upfield doublet is broadened, J_{AB} = 8.9 Hz, Δv_{AB} = 18.4 Hz, 2H), 4.49 (dd, J = 8.5, 3.9 Hz, 1H), 3.70-3.64 (m, 1H), 3.60-3.54 (m, 1H, assumed; partially obscured by water peak), 2.28-2.19 (m, 1H), 2.08-1.90 (m, 3H); 533.3 (chlorine isotope pattern observed) |
| 56 | 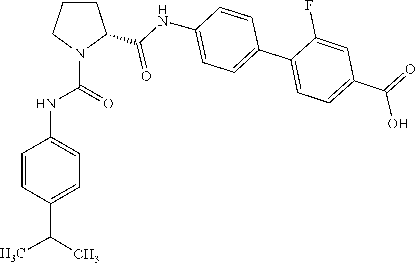 [SMILES: CC(C)c1ccc(NC(=O)N2CCC[C@@H]2C(=O)Nc2ccc(-c3ccc(C(=O)O)cc3F)cc2)cc1] | 10.17 (s, 1H), 8.20 (s, 1H), 7.83 (dd, J = 8.0, 1.6 Hz, 1H), 7.76-7.71 (m, 3H), 7.66 (t, J = 8.0 Hz, 1H), 7.56 (br d, J = 8.4 Hz, 2H), 7.39 (d, J = 8.5 Hz, 2H), 7.08 (d, J = 8.5 Hz, 2H), 4.47 (dd, J = 8.3, 3.7 Hz, 1H), 3.67-3.61 (m, 1H), 3.54-3.48 (m, 1H, assumed; partially obscured by water peak), 2.80 (septet, J = 6.9 Hz, 1H), 2.23-2.15 (m, 1H), 2.08-1.99 (m, 1H), 1.99-1.89 (m, 2H), 1.16 (d, J = 6.9 Hz, 6H); 490.4 |
| 57 | 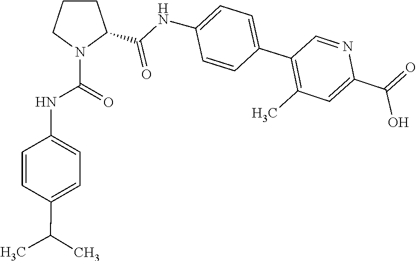 [SMILES: Cc1cc(C(=O)O)ncc1-c1ccc(NC(=O)[C@H]2CCCN2C(=O)Nc2ccc(C(C)C)cc2)cc1] | 10.16 (s, 1H), 8.48 (s, 1H), 8.20 (s, 1H), 8.00 (s, 1H), 7.75 (d, J = 8.6 Hz, 2H), 7.40 (d, J = 8.6 Hz, 2H), 7.40 (d, J = 8.6 Hz, 2H), 7.09 (d, J = 8.6 Hz, 2H), 4.48 (dd, J = 8.3, 3.7 Hz, 1H), 3.68-3.62 (m, 1H), 3.54-3.48 (m, 1H, assumed; partially obscured by water peak), 2.80 (septet, J = 7.0 Hz, 1H), 2.36 (s, 3H), 2.24-2.15 (m, 1H), 2.08-1.99 (m, 1H), 1.99- 1.90 (m, 2H), 1.16 (d, J = 6.9 Hz, 6H); 487.5 |
| 58 | 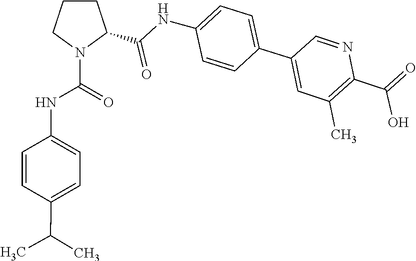 [SMILES: Cc1cc(-c2ccc(NC(=O)[C@H]3CCCN3C(=O)Nc3ccc(C(C)C)cc3)cc2)cnc1C(=O)O] | 10.18 (s, 1H), 8.78 (d, J = 2.2 Hz, 1H), 8.20 (s, 1H), 8.07 (br d, J = 2 Hz, 1H), 7.77 (AB quartet, J_{AB} = 9.2 Hz, Δv_{AB} = 7.5 Hz, 4H), 7.40 (d, J = 8.6 Hz, 2H), 7.09 (d, J = 8.6 Hz, 2H), 4.47 (dd, J = 8.3, 3.7 Hz, 1H), 3.65 (ddd, J = 9.3, 7.7, 4.7 Hz, 1H), 3.55-3.48 (m, 1H, assumed; partially obscured by water peak), 2.80 (septet, J = 7.0 Hz, 1H), 2.55 (s, 3H), 2.24-2.15 (m, 1H), 2.08-1.99 (m, 1H), 1.99- 1.89 (m, 2H), 1.16 (d, J = 6.9 Hz, 6H); 487.5 |
| 59 | 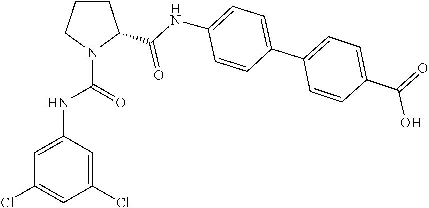 [SMILES: O=C(O)c1ccc(-c2ccc(NC(=O)[C@H]3CCCN3C(=O)Nc3cc(Cl)cc(Cl)c3)cc2)cc1] | 10.16 (s, 1H), 8.66 (s, 1H), 7.99 (d, J = 8.5 Hz, 2H), 7.78 (d, J = 8.5 Hz, 2H), 7.73 (AB quartet, J_{AB} = 8.9 Hz, Δv_{AB} = 12.5 Hz, 4H), 7.67 (d, J = 1.9 Hz, 2H), 7.11 (t, J = 1.9 Hz, 1H), 4.47 (dd, J = 8.3, 3.9 Hz, 1H), 3.67-3.61 (m, 1H), 3.57-3.50 (m, 1H), 2.26-2.17 (m, 1H), 2.07-1.89 (m, 3H); 498.3 (dichloro isotope pattern observed) |
| 60 | 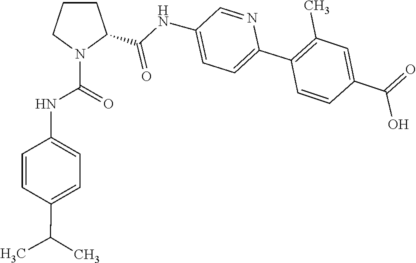 [SMILES: Cc1cc(C(=O)O)ccc1-c1ccc(NC(=O)[C@H]2CCCN2C(=O)Nc2ccc(C(C)C)cc2)cn1] | 10.35 (s, 1H), 8.88 (d, J = 2.6 Hz, 1H), 8.22 (s, 1H), 8.17 (dd, J = 8.5, 2.6 Hz, 1H), 7.87 (br s, 1H), 7.83 (br d, J = 8 Hz, 1H), 7.55 (d, J = 8.5 Hz, 1H), 7.50 (d, J = 7.9 Hz, 1H), 7.42-7.37 (m, 2H), 7.09 (d, J = 8.6 Hz, 2H), 4.48 (dd, J = 8.3, 3.8 Hz, 1H), 3.69-3.62 (m, 1H), 3.55- 3.49 (m, 1H, assumed; partially obscured by water peak), 2.80 (septet, J = 7.0 Hz, 1H), 2.37 (s, 3H), 2.25-2.17 (m, 1H), 2.08-2.00 (m, 1H), 2.00-1.91 (m, 2H), 1.16 (d, J = 6.9 Hz, 6H); 487.5 |
| 61 | 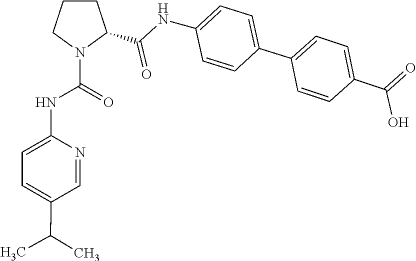 [SMILES: CC(C)c1ccc(NC(=O)N2CCC[C@@H]2C(=O)Nc2ccc(-c3ccc(C(=O)O)cc3)cc2)nc1] | 10.23 (s, 1H), 9.50 (br s, 1H), 8.11 (d, J = 2.3 Hz, 1H), 7.99 (d, J = 8.5 Hz, 2H), 7.96-7.86 (br m, 1H), 7.78 (d, J = 8.5 Hz, 2H), 7.75- 7.69 (m, 5H), 4.56-4.49 (m, 1H), 3.73-3.66 (m, 1H, assumed; partially obscured by water peak), 3.64-3.57 (m, 1H, assumed; partially obscured by water peak), 2.96-2.89 (m, 1H), 2.30-2.20 (m, 1H), 2.08-1.92 (m, 3H), 1.19 (d, J = 6.9 Hz, 6H); 473.3 |
| 62 | 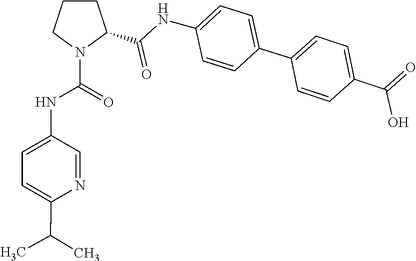 [SMILES: CC(C)c1ccc(NC(=O)N2CCC[C@@H]2C(=O)Nc2ccc(-c3ccc(C(=O)O)cc3)cc2)cn1] | 10.20 (s, 1H), 8.96 (br s, 1H), 8.82 (s, 1H), 8.29-8.19 (m, 1H), 7.99 (d, J = 8.4 Hz, 2H), 7.78 (d, J = 8.6 Hz, 2H), 7.72 (AB quartet, J_{AB} = 8.9 Hz, Δv_{AB} = 8.9 Hz, 4H), 7.67-7.59 (m, 1H), 4.50 (dd, J = 8.3, 3.8 Hz, 1H), 3.71- 3.63 (m, 1H, assumed; partially obscured by water peak), 3.59-3.53 (m, 1H, assumed; partially obscured by water peak), 3.12 (septet, J = 6.8 Hz, 1H, assumed; partially obscured by water peak), 2.28-2.20 (m, 1H), 2.09-1.92 (m, 3H), 1.26 (d, J = 6.9 Hz, 6H); 473.3 |
| 63 | 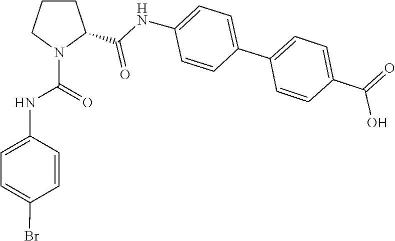 [SMILES: O=C(O)c1ccc(-c2ccc(NC(=O)[C@H]3CCCN3C(=O)Nc3ccc(Br)cc3)cc2)cc1] | 10.15 (s, 1H), 8.44 (s, 1H), 7.99 (d, J = 8.4 Hz, 2H), 7.78 (d, J = 8.5 Hz, 2H), 7.72 (AB quartet, J_{AB} = 8.9 Hz, Δv_{AB} = 14.0 Hz, 4H), 7.50 (d, J = 9.0 Hz, 2H), 7.39 (d, J = 9.0 Hz, 2H), 4.47 (dd, J = 8.3, 3.8 Hz, 1H), 3.68-3.61 (m, 1H), 3.56-3.49 (m, 1H, assumed; partially obscured by water peak), 2.24-2.16 (m, 1H), 2.07-1.89 (m, 3H); 508.2 (bromine isotope pattern observed) |
| 64 | 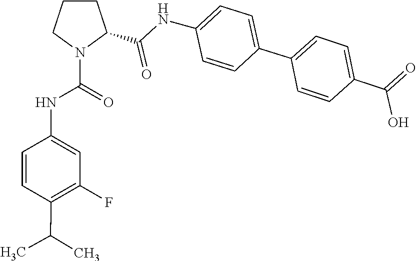 [SMILES: CC(C)c1ccc(NC(=O)N2CCC[C@@H]2C(=O)Nc2ccc(-c3ccc(C(=O)O)cc3)cc2)cc1F] | 10.15 (s, 1H), 8.40 (s, 1H), 7.99 (d, J = 8.4 Hz, 2H), 7.78 (d, J = 8.4 Hz, 2H), 7.72 (AB quartet, J_{AB} = 8.8 Hz, Δv_{AB} = 14.0 Hz, 4H), 7.40 (br d, J = 13.4 Hz, 1H), 7.23 (br d, half of AB quartet, J = 8.5 Hz, 1H), 7.16 (t, J = 8.7 Hz, 1H), 4.47 (dd, J = 8.4, 3.8 Hz, 1H), 3.67- 3.61 (m, 1H), 3.54-3.48 (m, 1H, assumed; partially obscured by water peak), 3.06 (septet, J = 6.9 Hz, 1H), 2.24-2.15 (m, 1H), 2.08-1.88 (m, 3H), 1.17 (d, J = 6.9 Hz, 6H); 490.3 |
| 65 | 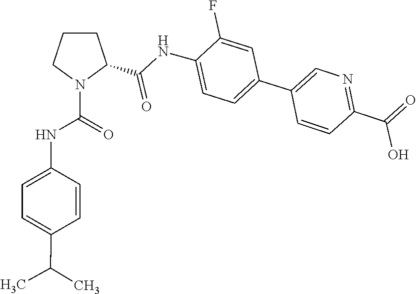 [SMILES: CC(C)c1ccc(NC(=O)N2CCC[C@@H]2C(=O)Nc2ccc(-c3ccc(C(=O)O)nc3)cc2F)cc1] | ¹H NMR, characteristic peaks: δ 10.04 (s, 1H), 9.06-9.03 (m, 1H), 8.29 (dd, J = 8.2, 2.4 Hz, 1H), 8.23 (s, 1H), 8.16-8.11 (m, 1H), 8.09 (d, J = 8.2 Hz, 1H), 7.82 (br d, J = 12.2 Hz, 1H), 7.66 (br d, J = 8.5 Hz, 1H), 7.42-7.37 (m, 2H), 7.10 (d, J = 8.5 Hz, 2H), 4.67-4.63 (m, 1H), 3.66-3.59 (m, 1H, assumed; partially obscured by water peak), 2.81 (septet, J = 6.9 Hz, 1H), 2.20-2.11 (m, 1H), 2.04-1.93 (m, 3H), 1.16 (d, J = 6.9 Hz, 6H); 491.4 |
| 66 | 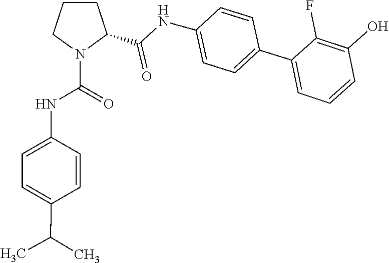 [SMILES: CC(C)c1ccc(NC(=O)N2CCC[C@@H]2C(=O)Nc2ccc(-c3cccc(O)c3F)cc2)cc1] | 3.01 minutes¹; 462.4 |
| 67 | 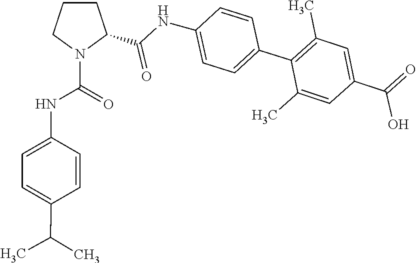 [SMILES: Cc1cc(C(=O)O)cc(C)c1-c1ccc(NC(=O)[C@H]2CCCN2C(=O)Nc2ccc(C(C)C)cc2)cc1] | 3.10 minutes¹; 500.4 |
| 68 | 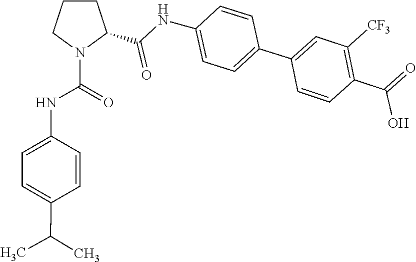 [SMILES: CC(C)c1ccc(NC(=O)N2CCC[C@@H]2C(=O)Nc2ccc(-c3ccc(C(=O)O)c(C(F)(F)F)c3)cc2)cc1] | 3.12 minutes¹; 540.4 |
| 69 | 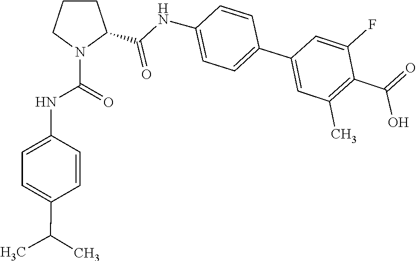 [SMILES: Cc1cc(-c2ccc(NC(=O)[C@H]3CCCN3C(=O)Nc3ccc(C(C)C)cc3)cc2)cc(F)c1C(=O)O] | 3.04 minutes¹; 504.4 |
| 70 | 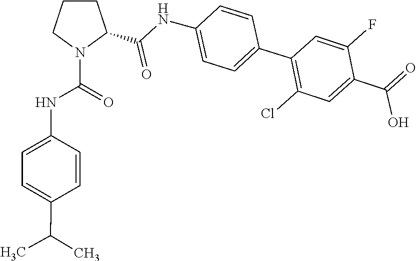 [SMILES: CC(C)c1ccc(NC(=O)N2CCC[C@@H]2C(=O)Nc2ccc(-c3cc(F)c(C(=O)O)cc3Cl)cc2)cc1] | 3.10 minutes¹; 524.4 (chlorine isotope pattern observed) |
| 71 | 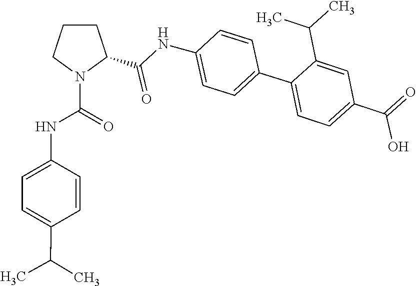 [SMILES: CC(C)c1ccc(NC(=O)N2CCC[C@@H]2C(=O)Nc2ccc(-c3ccc(C(=O)O)cc3C(C)C)cc2)cc1] | 3.21 minutes¹; 514.5 |
| 72 | 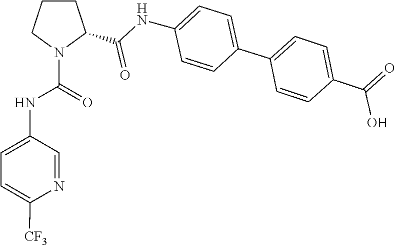 [SMILES: O=C(O)c1ccc(-c2ccc(NC(=O)[C@H]3CCCN3C(=O)Nc3ccc(C(F)(F)F)nc3)cc2)cc1] | 2.46 minutes¹; 499.2 |
| 73 | 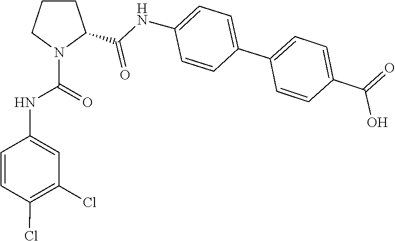 [SMILES: O=C(O)c1ccc(-c2ccc(NC(=O)[C@H]3CCCN3C(=O)Nc3ccc(Cl)c(Cl)c3)cc2)cc1] | 10.16 (s, 1H), 8.61 (s, 1H), 7.99 (d, J = 8.5 Hz, 2H), 7.91 (d, J = 2.5 Hz, 1H), 7.78 (d, J = 8.5 Hz, 2H), 7.72 (AB quartet, J_{AB} = 8.9 Hz, Δv_{AB} = 14.5 Hz, 4H), 7.52 (dd, component of ABX system, J = 8.9, 2.5 Hz, 1H), 7.47 (d, half of AB quartet, J = 8.9 Hz, 1H), 4.47 (dd, J = 8.3, 3.8 Hz, 1H), 3.64 (ddd, J = 9.2, 7.5, 4.8 Hz, 1H), 3.57-3.50 (m, 1H), 2.26-2.17 (m, 1H), 2.08-1.89 (m, 3H); 498.3 (dichloro isotope pattern observed) |
| 74 | 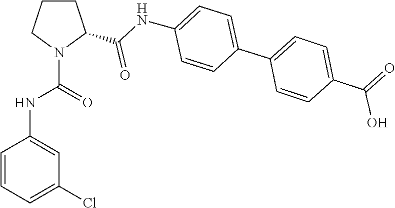 [SMILES: O=C(O)c1ccc(-c2ccc(NC(=O)[C@H]3CCCN3C(=O)Nc3cccc(Cl)c3)cc2)cc1] | 10.16 (s, 1H), 8.49 (s, 1H), 7.99 (d, J = 8.5 Hz, 2H), 7.78 (d, J = 8.5 Hz, 2H), 7.75-7.69 (m, 5H), 7.46-7.43 (m, 1H), 7.24 (t, J = 8.1 Hz, 1H), 6.97 (ddd, J = 7.9, 2.1, 0.9 Hz, 1H), 4.47 (dd, J = 8.4, 3.9 Hz, 1H), 3.64 (ddd, J = 9.3, 7.6, 4.9 Hz, 1H), 3.56-3.50 (m, 1H, assumed; partially obscured by water peak), 2.25-2.16 (m, 1H), 2.07-1.89 (m, 3H); 464.4 (chlorine isotope pattern observed) |
| 75 | 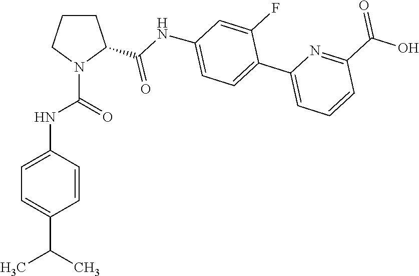 [SMILES: CC(C)c1ccc(NC(=O)N2CCC[C@@H]2C(=O)Nc2ccc(-c3cccc(C(=O)O)n3)c(F)c2)cc1] | 10.40 (s, 1H), 8.22 (s, 1H), 8.09-8.03 (m, 2H), 8.00-7.96 (m, 2H), 7.80 (dd, J = 14.3, 2.0 Hz, 1H), 7.46 (dd, J = 8.6, 2.0 Hz, 1H), 7.40 (d, J = 8.6 Hz, 2H), 7.09 (d, J = 8.6 Hz, 2H), 4.46 (dd, J = 8.3, 3.8 Hz, 1H), 3.65 (ddd, J = 9.3, 7.6, 4.7 Hz, 1H), 3.55-3.49 (m, 1H), 2.80 (septet, J = 6.9 Hz, 1H), 2.25-2.15 (m, 1H), 2.08-2.00 (m, 1H), 2.00-1.90 (m, 2H), 1.16 (d, J = 6.9 Hz, 6H); 491.4 |
| 76 | 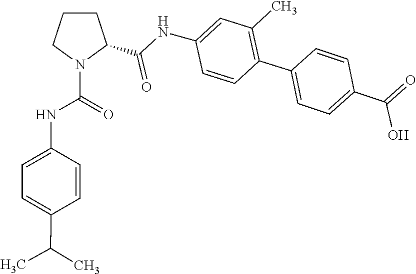 [SMILES: Cc1cc(NC(=O)[C@H]2CCCN2C(=O)Nc2ccc(C(C)C)cc2)ccc1-c1ccc(C(=O)O)cc1] | δ 10.01 (s, 1H), 8.18 (s, 1H), 7.98 (d, J = 8.4 Hz, 2H), 7.58 (br s, 1H), 7.52 (dd, J = 8.3, 2.2 Hz, 1H), 7.44 (d, J = 8.4 Hz, 2H), 7.40 (d, J = 8.4 Hz, 2H), 7.17 (d, J = 8.3 Hz, 1H), 7.09 (d, J = 8.5 Hz, 2H), 4.46 (dd, J = 8.4, 3.6 Hz, 1H), 3.67-3.61 (m, 1H), 3.54-3.47 (m, 1H), 2.80 (septet, J = 6.9 Hz, 1H), 2.22 (s, 3H), 2.21- 2.13 (m, 1H), 2.07-1.88 (m, 3H), 1.16 (d, J = 6.9 Hz, 6H); 486.5 |
| 77 | 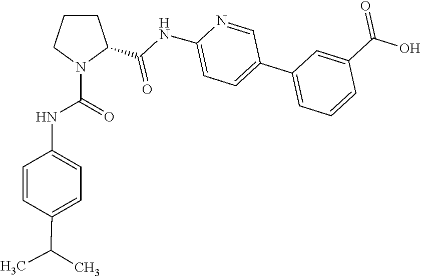 [SMILES: CC(C)c1ccc(NC(=O)N2CCC[C@@H]2C(=O)Nc2ccc(-c3cccc(C(=O)O)c3)cn2)cc1] | 10.64 (s, 1H), 8.71-8.65 (m, 1H), 8.22 (s, 1H), 8.20 (br s, 1H), 8.17 (d, half of AB quartet, J = 8.7 Hz, 1H), 8.14 (dd, component of ABX system, J = 8.7, 2.4 Hz, 1H), 7.98- 7.93 (m, 2H), 7.61 (t, J = 7.8 Hz, 1H), 7.39 (d, J = 8.7 Hz, 2H), 7.09 (d, J = 8.7 Hz, 2H), 4.62 (dd, J = 8.6, 3.4 Hz, 1H), 3.67-3.60 (m, 1H), 3.54-3.48 (m, 1H), 2.80 (septet, J = 6.9 Hz, 1H), 2.21-2.13 (m, 1H), 2.05-1.91 (m, 3H), 1.16 (d, J = 6.9 Hz, 6H); 473.5 |
| 78 | 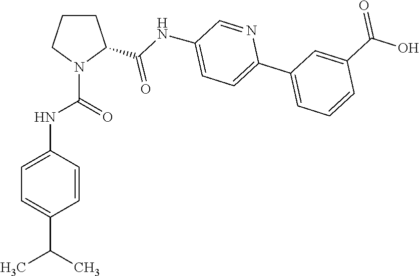 [SMILES: CC(C)c1ccc(NC(=O)N2CCC[C@@H]2C(=O)Nc2ccc(-c3cccc(C(=O)O)c3)nc2)cc1] | 10.35 (s, 1H), 8.87 (d, J = 2.6 Hz, 1H), 8.63 (t, J = 1.9 Hz, 1H), 8.28-8.25 (m, 1H), 8.22 (s, 1H), 8.19 (dd, J = 8.7, 2.6 Hz, 1H), 8.00 (d, J = 8.7 Hz, 1H), 7.95 (dt, J = 7.7, 1.4 Hz, 1H), 7.60 (t, J = 7.8 Hz, 1H), 7.40 (d, J = 8.6 Hz, 2H), 7.09 (d, J = 8.7 Hz, 2H), 4.48 (dd, J = 8.4, 3.8 Hz, 1H), 3.69-3.62 (m, 1H), 3.55-3.49 (m, 1H, assumed; partially obscured by water peak), 2.79 (septet, J = 6.9 Hz, 1H), 2.25- 2.16 (m, 1H), 2.09-1.90 (m, 3H), 1.16 (d, J = 6.9 Hz, 6H); 473.5 |
| 79 | 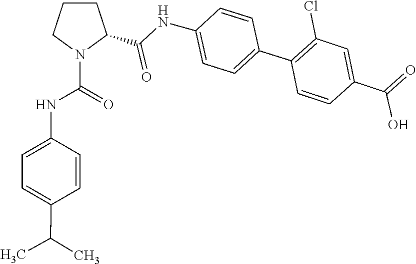 [SMILES: CC(C)c1ccc(NC(=O)N2CCC[C@@H]2C(=O)Nc2ccc(-c3ccc(C(=O)O)cc3Cl)cc2)cc1] | 3.16 minutes¹; 506.4 (chlorine isotope pattern observed) |
| 80 | 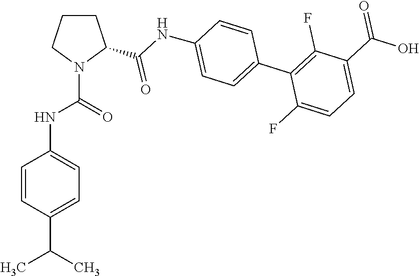 [SMILES: CC(C)c1ccc(NC(=O)N2CCC[C@@H]2C(=O)Nc2ccc(-c3c(F)ccc(C(=O)O)c3F)cc2)cc1] | 3.06 minutes¹; 508.4 |
| 81 | 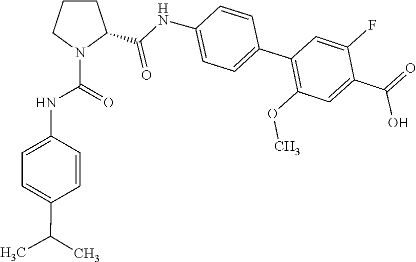 [SMILES: COc1cc(C(=O)O)c(F)cc1-c1ccc(NC(=O)[C@H]2CCCN2C(=O)Nc2ccc(C(C)C)cc2)cc1] | 3.05 minutes¹; 520.4 |
| 82 | 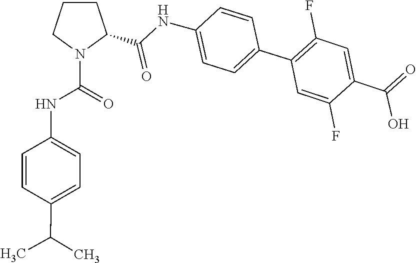 [SMILES: CC(C)c1ccc(NC(=O)N2CCC[C@@H]2C(=O)Nc2ccc(-c3cc(F)c(C(=O)O)cc3F)cc2)cc1] | 3.09 minutes¹; 508.4 |
| 83 | 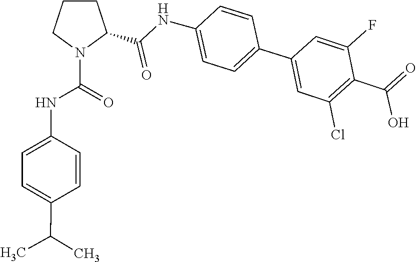 [SMILES: CC(C)c1ccc(NC(=O)N2CCC[C@@H]2C(=O)Nc2ccc(-c3cc(F)c(C(=O)O)c(Cl)c3)cc2)cc1] | 3.15 minutes¹; 524.3 (chlorine isotope pattern observed) |
| 84 | 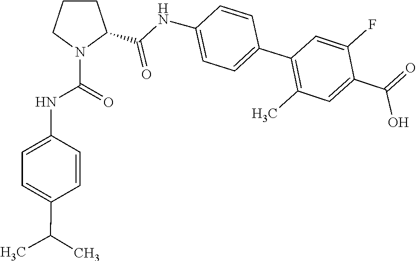 [SMILES: Cc1cc(C(=O)O)c(F)cc1-c1ccc(NC(=O)[C@H]2CCCN2C(=O)Nc2ccc(C(C)C)cc2)cc1] | 3.11 minutes¹; 504.4 |
| 85 | 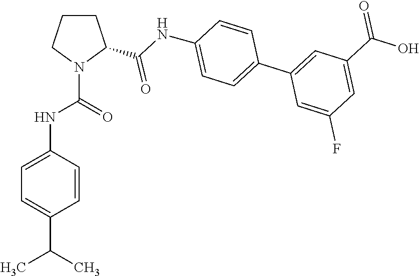 [SMILES: CC(C)c1ccc(NC(=O)N2CCC[C@@H]2C(=O)Nc2ccc(-c3cc(F)cc(C(=O)O)c3)cc2)cc1] | 3.15 minutes¹; 490.4 |
| 86 | 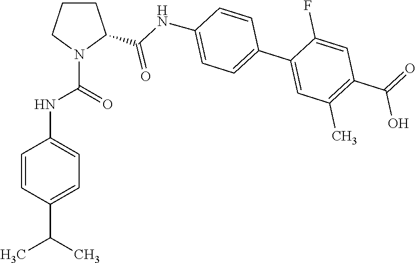 [SMILES: Cc1cc(-c2ccc(NC(=O)[C@H]3CCCN3C(=O)Nc3ccc(C(C)C)cc3)cc2)c(F)cc1C(=O)O] | 3.19 minutes¹; 504.4 |
| 87 | 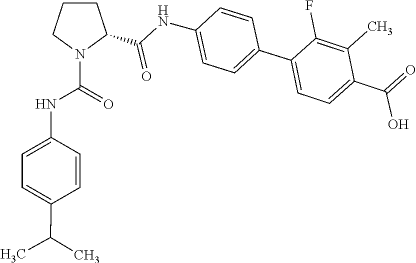 [SMILES: Cc1c(C(=O)O)ccc(-c2ccc(NC(=O)[C@H]3CCCN3C(=O)Nc3ccc(C(C)C)cc3)cc2)c1F] | 3.17 minutes¹; 504.4 |
| 88 | 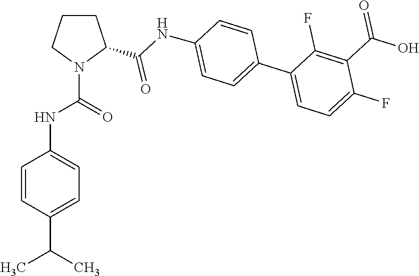 [SMILES: CC(C)c1ccc(NC(=O)N2CCC[C@@H]2C(=O)Nc2ccc(-c3ccc(F)c(C(=O)O)c3F)cc2)cc1] | 3.04 minutes¹; 508.4 |
| 89 | 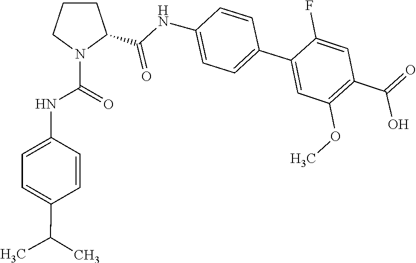 [SMILES: COc1cc(-c2ccc(NC(=O)[C@H]3CCCN3C(=O)Nc3ccc(C(C)C)cc3)cc2)c(F)cc1C(=O)O] | 3.06 minutes¹; 520.4 |
| 90 | 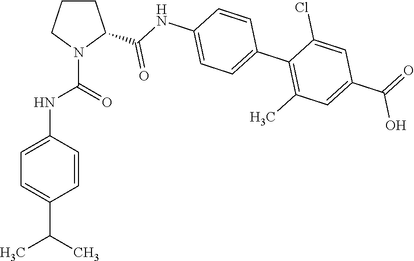 [SMILES: Cc1cc(C(=O)O)cc(Cl)c1-c1ccc(NC(=O)[C@H]2CCCN2C(=O)Nc2ccc(C(C)C)cc2)cc1] | 3.23 minutes¹; 520.4 (chlorine isotope pattern observed) |
| 91 | 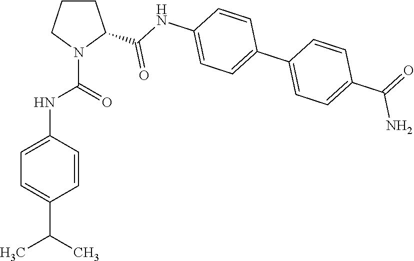 [SMILES: CC(C)c1ccc(NC(=O)N2CCC[C@@H]2C(=O)Nc2ccc(-c3ccc(C(N)=O)cc3)cc2)cc1] | 2.60 minutes¹; 471.4 |
| 92 | 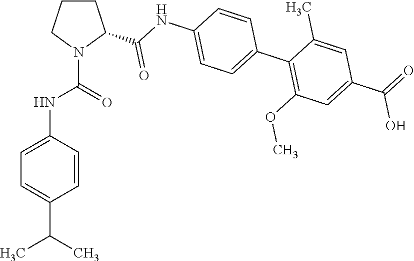 [SMILES: COc1cc(C(=O)O)cc(C)c1-c1ccc(NC(=O)[C@H]2CCCN2C(=O)Nc2ccc(C(C)C)cc2)cc1] | 2.85 minutes¹; 516.3 |
| 93 | 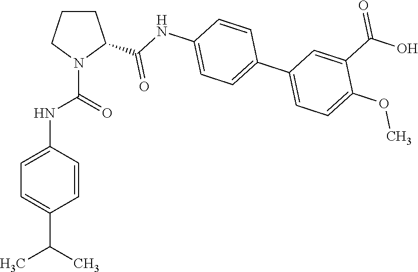 [SMILES: COc1ccc(-c2ccc(NC(=O)[C@H]3CCCN3C(=O)Nc3ccc(C(C)C)cc3)cc2)cc1C(=O)O] | 2.75 minutes¹; 502.3 |
| 94 | 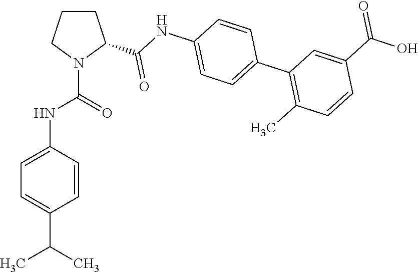 [SMILES: Cc1ccc(C(=O)O)cc1-c1ccc(NC(=O)[C@H]2CCCN2C(=O)Nc2ccc(C(C)C)cc2)cc1] | 2.89 minutes¹; 486.3 |
| 95 | 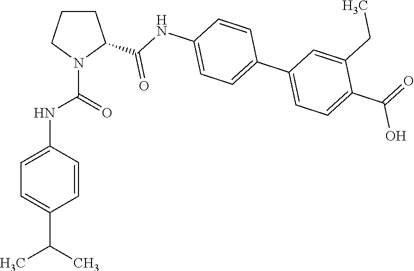 [SMILES: CCc1cc(-c2ccc(NC(=O)[C@H]3CCCN3C(=O)Nc3ccc(C(C)C)cc3)cc2)ccc1C(=O)O] | 2.98 minutes¹; 500.3 |
| 96 | 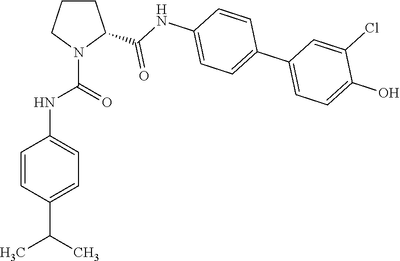 [SMILES: CC(C)c1ccc(NC(=O)N2CCC[C@@H]2C(=O)Nc2ccc(-c3ccc(O)c(Cl)c3)cc2)cc1] | 2.98 minutes¹; 478.3 (chlorine isotope pattern observed) |
| 97 | 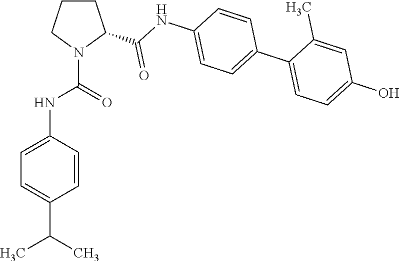 [SMILES: Cc1cc(O)ccc1-c1ccc(NC(=O)[C@H]2CCCN2C(=O)Nc2ccc(C(C)C)cc2)cc1] | 2.89 minutes¹; 458.3 |
| 98 | 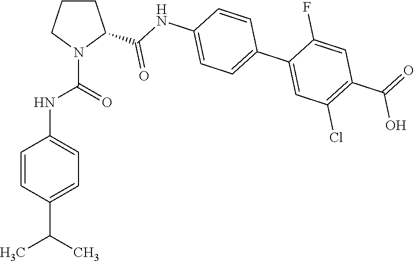 [SMILES: CC(C)c1ccc(NC(=O)N2CCC[C@@H]2C(=O)Nc2ccc(-c3cc(Cl)c(C(=O)O)cc3F)cc2)cc1] | 2.96 minutes¹; 524.2 (chlorine isotope pattern observed) |
| 99 | 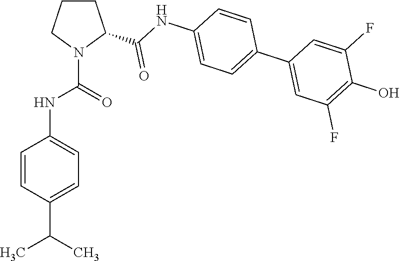 [SMILES: CC(C)c1ccc(NC(=O)N2CCC[C@@H]2C(=O)Nc2ccc(-c3cc(F)c(O)c(F)c3)cc2)cc1] | 2.94 minutes¹; 480.3 |
| 100 | 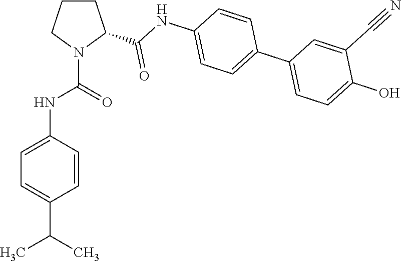 [SMILES: CC(C)c1ccc(NC(=O)N2CCC[C@@H]2C(=O)Nc2ccc(-c3ccc(O)c(C#N)c3)cc2)cc1] | 2.84 minutes¹; 469.3 |
| 101 | 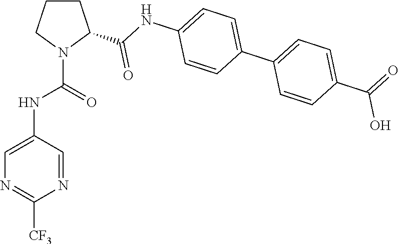 [SMILES: O=C(O)c1ccc(-c2ccc(NC(=O)[C@H]3CCCN3C(=O)Nc3cnc(C(F)(F)F)nc3)cc2)cc1] | 2.46 minutes¹; 500.3 |
| 102 | 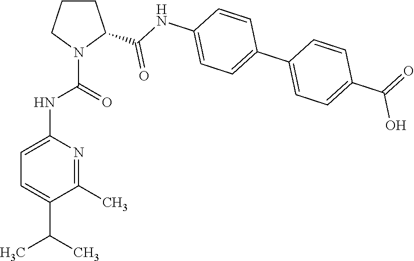 [SMILES: Cc1nc(NC(=O)N2CCC[C@@H]2C(=O)Nc2ccc(-c3ccc(C(=O)O)cc3)cc2)ccc1C(C)C] | 2.25 minutes¹; 487.5 |
| 103 | 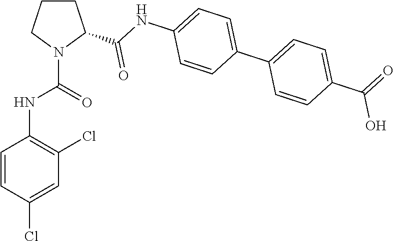 [SMILES: O=C(O)c1ccc(-c2ccc(NC(=O)[C@H]3CCCN3C(=O)Nc3ccc(Cl)cc3Cl)cc2)cc1] | 12.92 (v br s, 1H), 10.17 (s, 1H), 7.99 (d, J = 8.1 Hz, 2H), 7.85 (br s, 1H), 7.78 (d, J = 8.2 Hz, 2H), 7.75-7.69 (m, 5H), 7.61 (d, J = 2.4 Hz, 1H), 7.36 (dd, J = 8.8, 2.4 Hz, 1H), 4.49 (dd, J = 8.7, 3.4 Hz, 1H), 3.70-3.64 (m, 1H), 3.59-3.53 (m, 1H), 2.27-2.18 (m, 1H), 2.09- 1.93 (m, 3H); 498.3 (dichloro isotope pattern observed) |
| 104 | 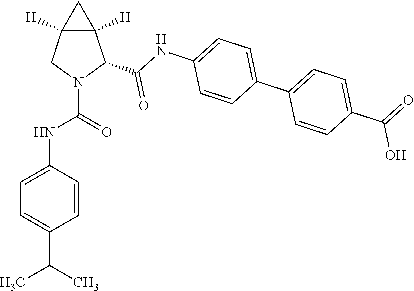 [SMILES: CC(C)c1ccc(NC(=O)N2C[C@H]3C[C@H]3[C@@H]2C(=O)Nc2ccc(-c3ccc(C(=O)O)cc3)cc2)cc1] | 12.92 (v br s, 1H), 10.33 (s, 1H), 8.16 (s, 1H), 7.99 (d, J = 8.4 Hz, 2H), 7.78 (d, J = 8.4 Hz, 2H), 7.74 (AB quartet, J_{AB} = 8.8 Hz, Δv_{AB} = 24.0 Hz, 4H), 7.37 (d, J = 8.5 Hz, 2H), 7.08 (d, J = 8.6 Hz, 2H), 4.55 (s, 1H), 3.73 (d, half of AB quartet, J = 9.3 Hz, 1H), 3.70 (dd, component of ABX system, J = 9.3, 3.9 Hz, 1H), 2.79 (septet, J = 6.9 Hz, 1H), 1.76-1.70 (m, 1H), 1.68-1.63 (m, 1H), 1.15 (d, J = 6.9 Hz, 6H), 0.82-0.76 (m, 1H), 0.33-0.28 (m, 1H); 484.4 |
| 105 | 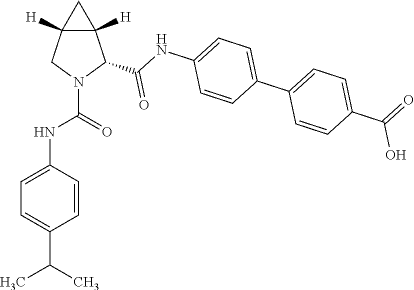 [SMILES: CC(C)c1ccc(NC(=O)N2C[C@@H]3C[C@@H]3[C@@H]2C(=O)Nc2ccc(-c3ccc(C(=O)O)cc3)cc2)cc1] | 12.92 (v br s, 1H), 10.04 (s, 1H), 8.15 (s, 1H), 7.99 (d, J = 8.2 Hz, 2H), 7.78 (d, J = 8.3 Hz, 2H), 7.73 (AB quartet, J_{AB} = 8.7 Hz, Δv_{AB} = 19.2 Hz, 4H), 7.36 (d, J = 8.3 Hz, 2H), 7.08 (d, J = 8.3 Hz, 2H), 4.49 (d, J = 5.2 Hz, 1H), 3.83 (d, J = 9.3 Hz, 1H), 3.58 (dd, J = 9.3, 5.1 Hz, 1H), 2.79 (septet, J = 6.9 Hz, 1H), 1.97-1.89 (m, 1H), 1.80-1.73 (m, 1H), 1.15 (d, J = 6.9 Hz, 6H), 0.82-0.78 (m, 1H), 0.65-0.59 (m, 1H); 484.4 |
| 106 | 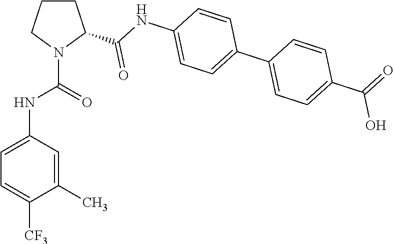 [SMILES: Cc1cc(NC(=O)N2CCC[C@@H]2C(=O)Nc2ccc(-c3ccc(C(=O)O)cc3)cc2)ccc1C(F)(F)F] | 2.83 minutes¹; 512.2 |
| 107 | 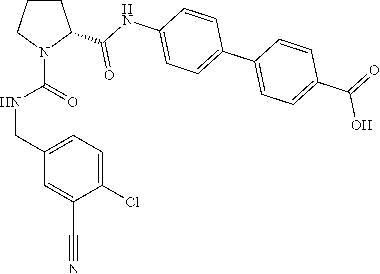 [SMILES: N#Cc1cc(CNC(=O)N2CCC[C@@H]2C(=O)Nc2ccc(-c3ccc(C(=O)O)cc3)cc2)ccc1Cl] | 2.49 minutes¹; 503.2 (chlorine isotope pattern observed) |
| 108 | 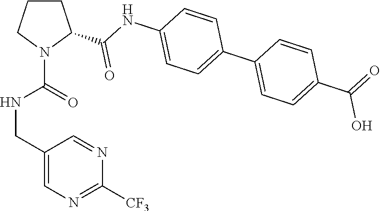 [SMILES: O=C(O)c1ccc(-c2ccc(NC(=O)[C@H]3CCCN3C(=O)NCc3cnc(C(F)(F)F)nc3)cc2)cc1] | 2.31 minutes¹; 514.2 |
| 109 | 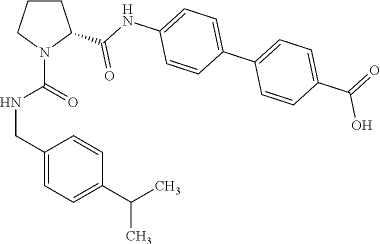 [SMILES: CC(C)c1ccc(CNC(=O)N2CCC[C@@H]2C(=O)Nc2ccc(-c3ccc(C(=O)O)cc3)cc2)cc1] | 2.80 minutes¹; 486.3 |
| 110 | 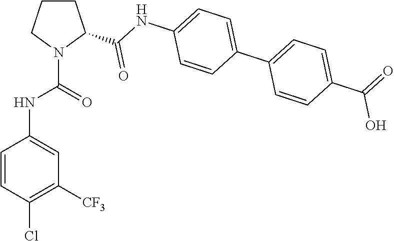 [SMILES: O=C(O)c1ccc(-c2ccc(NC(=O)[C@H]3CCCN3C(=O)Nc3ccc(Cl)c(C(F)(F)F)c3)cc2)cc1] | 2.87 minutes¹; 532.3 (chlorine isotope pattern observed) |
| 111 | 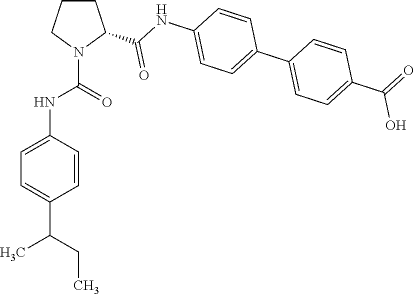 [SMILES: CCC(C)c1ccc(NC(=O)N2CCC[C@@H]2C(=O)Nc2ccc(-c3ccc(C(=O)O)cc3)cc2)cc1] | 2.95 minutes¹; 486.3 |
| 112 | 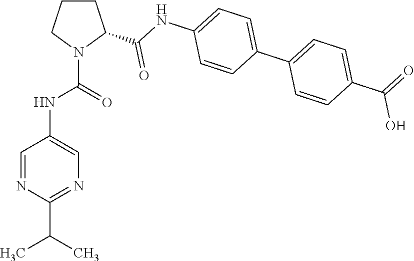 [SMILES: CC(C)c1ncc(NC(=O)N2CCC[C@@H]2C(=O)Nc2ccc(-c3ccc(C(=O)O)cc3)cc2)cn1] | 2.17 minutes¹; 474.3 |
| 113 | 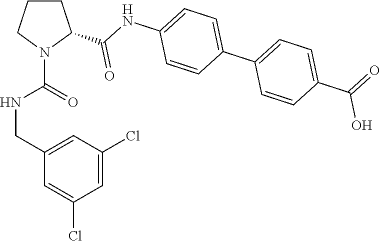 [SMILES: O=C(O)c1ccc(-c2ccc(NC(=O)[C@H]3CCCN3C(=O)NCc3cc(Cl)cc(Cl)c3)cc2)cc1] | 2.76 minutes¹; 512.2 (dichloro isotope pattern observed) |
| 114 | 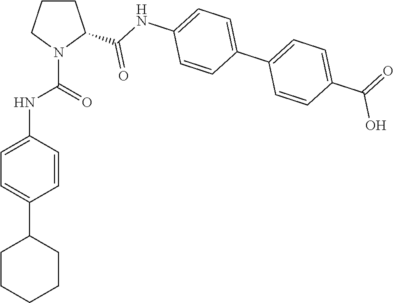 [SMILES: O=C(O)c1ccc(-c2ccc(NC(=O)[C@H]3CCCN3C(=O)Nc3ccc(C4CCCCC4)cc3)cc2)cc1] | ¹H NMR (400 MHz, DMSO-d₆), characteristic peaks: δ 12.89 (br s, 1H), 10.13 (s, 1H), 8.18 (s, 1H), 7.99 (d, J = 8.5 Hz, 2H), 7.78 (d, J = 8.5 Hz, 2H), 7.72 (AB quartet, J_{AB} = 9.0 Hz, Δv_{AB} = 11.8 Hz, 4H), 7.39 (d, J = 8.6 Hz, 2H), 7.06 (d, J = 8.6 Hz, 2H), 4.47 (dd, J = 8.3, 3.6 Hz, 1H), 3.69-3.60 (m, 1H), 3.56-3.46 (m, 1H), 2.44-2.35 (m, 1H), 2.25-2.13 (m, 1H), 1.81-1.63 (m, 5H), 1.42-1.26 (m, 4H); 512.4 |
| 115 | 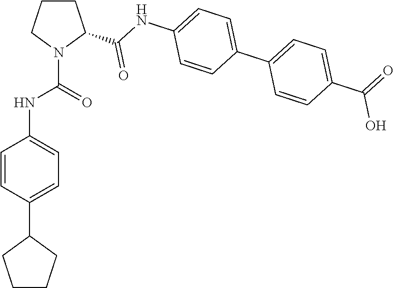 [SMILES: O=C(O)c1ccc(-c2ccc(NC(=O)[C@H]3CCCN3C(=O)Nc3ccc(C4CCCC4)cc3)cc2)cc1] | 10.14 (s, 1H), 8.20 (s, 1H), 7.99 (d, J = 8.2 Hz, 2H), 7.78 (d, J = 8.2 Hz, 2H), 7.75-7.72 (m, 2H), 7.70 (d, half of AB quartet, J = 8.6 Hz, 2H), 7.41-7.36 (m, 2H), 7.09 (d, J = 8.3 Hz, 2H), 4.49-4.44 (m, 1H), 3.68-3.60 (m, 1H), 3.54-3.48 (m, 1H, assumed; partially obscured by water peak), 2.93-2.83 (m, 1H), 2.23-2.14 (m, 1H), 2.07-1.99 (m, 1H), 1.99- 1.89 (m, 4H), 1.78-1.69 (m, 2H), 1.66- 1.56 (m, 2H), 1.53-1.42 (m, 2H); 498.4 |
| 116 | 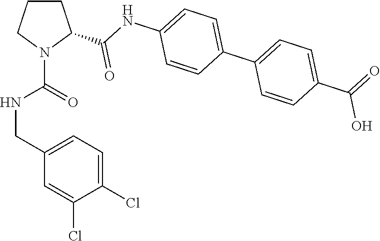 [SMILES: O=C(O)c1ccc(-c2ccc(NC(=O)[C@H]3CCCN3C(=O)NCc3ccc(Cl)c(Cl)c3)cc2)cc1] | 2.71 minutes¹; 512.1 (dichloro isotope pattern observed) |
| 117 | 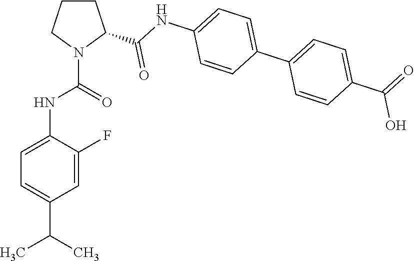 [SMILES: CC(C)c1ccc(NC(=O)N2CCC[C@@H]2C(=O)Nc2ccc(-c3ccc(C(=O)O)cc3)cc2)c(F)c1] | 12.92 (v br s, 1H), 10.14 (s, 1H), 7.99 (d, J = 8.5 Hz, 2H), 7.93 (s, 1H), 7.78 (d, J = 8.5 Hz, 2H), 7.71 (AB quartet, J_{AB} = 9.0 Hz, Δv_{AB} = 9.7 Hz, 4H), 7.39 (t, J = 8.3 Hz, 1H), 7.06 (dd, J = 12.1, 2.0 Hz, 1H), 6.98 (dd, J = 8.3, 2.0 Hz, 1H), 4.47 (dd, J = 8.4, 3.6 Hz, 1H), 3.67-3.61 (m, 1H), 3.55-3.48 (m, 1H), 2.86 (septet, J = 6.9 Hz, 1H), 2.24-2.15 (m, 1H), 2.08-1.99 (m, 1H), 1.99-1.91 (m, 2H), 1.17 (d, J = 6.9 Hz, 6H); 490.4 |
| 118 | 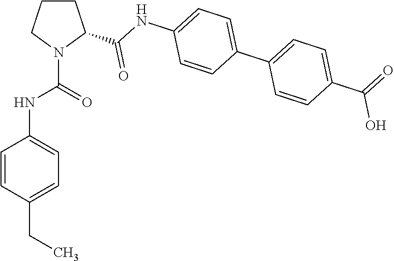 [SMILES: CCc1ccc(NC(=O)N2CCC[C@@H]2C(=O)Nc2ccc(-c3ccc(C(=O)O)cc3)cc2)cc1] | ¹H NMR, characteristic peaks: δ 10.14 (s, 1H), 8.19 (s, 1H), 7.98 (d, J = 8.4 Hz, 2H), 7.78 (d, J = 8.4 Hz, 2H), 7.72 (AB quartet, J_{AB} = 8.8 Hz, Δv_{AB} = 15.2 Hz, 4H), 7.39 (d, J = 8.2 Hz, 2H), 7.05 (d, J = 8.3 Hz, 2H), 4.46 (dd, J = 8.3, 3.8 Hz, 1H), 3.68-3.60 (m, 1H, assumed; partially obscured by water peak), 2.23-2.14 (m, 1H), 2.07-1.99 (m, 1H), 1.99-1.88 (m, 2H), 1.13 (t, J = 7.6 Hz, 3H); 458.4 |
| 119 | 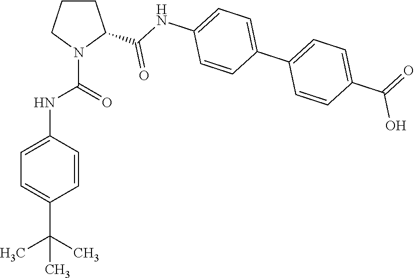 [SMILES: CC(C)(C)c1ccc(NC(=O)N2CCC[C@@H]2C(=O)Nc2ccc(-c3ccc(C(=O)O)cc3)cc2)cc1] | ¹H NMR, characteristic peaks: δ 10.14 (s, 1H), 8.20 (s, 1H), 7.98 (d, J = 8.4 Hz, 2H), 7.78 (d, J = 8.5 Hz, 2H), 7.72 (AB quartet, J_{AB} = 8.9 Hz, Δv_{AB} = 14.2 Hz, 4H), 7.39 (d, J = 8.7 Hz, 2H), 7.23 (d, J = 8.8 Hz, 2H), 4.47 (dd, J = 8.3, 3.7 Hz, 1H), 2.22-2.14 (m, 1H), 2.07-1.99 (m, 1H), 1.99-1.89 (m, 2H), 1.23 (s, 9H); 486.4 |
| 120 | 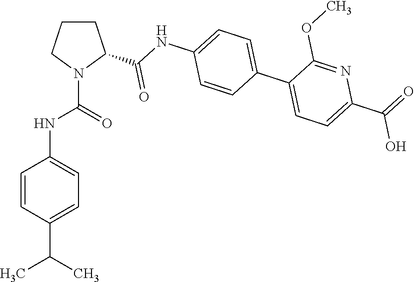 [SMILES: COc1nc(C(=O)O)ccc1-c1ccc(NC(=O)[C@H]2CCCN2C(=O)Nc2ccc(C(C)C)cc2)cc1] | 10.13 (s, 1H), 8.19 (s, 1H), 7.88 (d, J = 7.5 Hz, 1H), 7.73 (d, J = 7.5 Hz, 1H), 7.69 (d, J = 8.4 Hz, 2H), 7.57 (d, J = 8.6 Hz, 2H), 7.41-7.37 (m, 2H), 7.09 (d, J = 8.5 Hz, 2H), 4.47 (dd, J = 8.5, 3.7 Hz, 1H), 3.94 (s, 3H), 3.68-3.61 (m, 1H, assumed; partially obscured by water peak), 3.53-3.47 (m, 1H, assumed; partially obscured by water peak), 2.79 (septet, J = 6.8 Hz, 1H), 2.23-2.14 (m, 1H), 2.07-1.98 (m, 1H), 1.98-1.88 (m, 2H), 1.16 (d, J = 6.9 Hz, 6H); 503.3 |
| 121 | 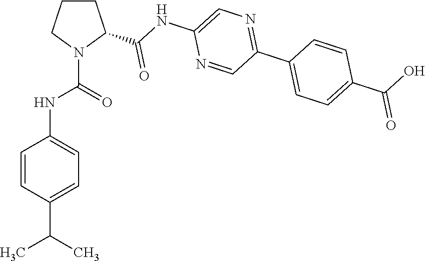 [SMILES: CC(C)c1ccc(NC(=O)N2CCC[C@@H]2C(=O)Nc2cnc(-c3ccc(C(=O)O)cc3)cn2)cc1] | 11.03 (s, 1H), 9.41 (s, 1H), 9.10-9.08 (m, 1H), 8.24 (s, 1H), 8.23-8.20 (m, 2H), 8.05 (d, J = 8.1 Hz, 2H), 7.41-7.36 (m, 2H), 7.09 (d, J = 8.2 Hz, 2H), 4.68-4.60 (m, 1H), 3.69- 3.60 (m, 1H, assumed; partially obscured by water peak), 3.55-3.48 (m, 1H, assumed; partially obscured by water peak), 2.84-2.76 (m, 1H), 2.24-2.15 (m, 1H), 2.07-1.92 (m, 3H), 1.16 (br d, J = 6.9 Hz, 6H); 474.4 |
| 122 | 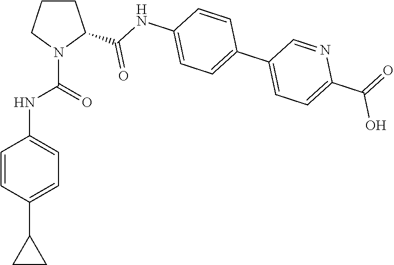 [SMILES: O=C(O)c1ccc(-c2ccc(NC(=O)[C@H]3CCCN3C(=O)Nc3ccc(C4CC4)cc3)cc2)cn1] | ¹H NMR, characteristic peaks: δ 10.19 (s, 1H), 9.01 (br s, 1H), 8.24 (dd, J = 8.2, 2.4 Hz, 1H), 8.19 (s, 1H), 8.08 (d, J = 8.2 Hz, 1H), 7.78 (AB quartet, J_{AB} = 8.9 Hz, Δv_{AB} = 10.5 Hz, 4H), 7.39-7.34 (m, 2H), 6.93 (d, J = 8.4 Hz, 2H), 4.46 (dd, J = 8.4, 3.7 Hz, 1H), 3.67-3.60 (m, 1H, assumed; partially obscured by water peak), 2.23-2.15 (m, 1H), 2.07-1.98 (m, 1H), 1.98-1.89 (m, 2H), 1.85-1.79 (m, 1H), 0.89-0.83 (m, 2H), 0.59-0.55 (m, 2H); 471.4 |
| 123 | 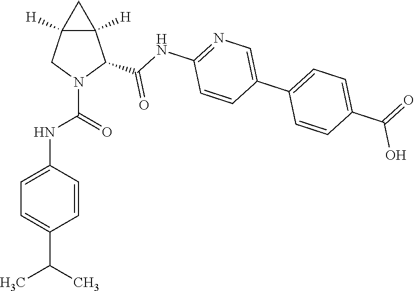 [SMILES: CC(C)c1ccc(NC(=O)N2C[C@H]3C[C@H]3[C@@H]2C(=O)Nc2ccc(-c3ccc(C(=O)O)cc3)cn2)cc1] | 10.86 (s, 1H), 8.76-8.73 (m, 1H), 8.20-8.15 (m, 3H), 8.02 (d, J = 8.4 Hz, 2H), 7.86 (d, J = 8.3 Hz, 2H), 7.35 (br d, J = 8.2 Hz, 2H), 7.08 (d, J = 8.6 Hz, 2H), 4.69 (s, 1H), 3.73 (d, half of AB quartet, J = 9.3 Hz, 1H), 3.69 (dd, component of ABX system, J = 9.3, 4.0 Hz, 1H), 2.79 (septet, J = 6.9 Hz, 1H), 1.74-1.68 (m, 1H), 1.68-1.63 (m, 1H), 1.15 (d, J = 6.9 Hz, 6H), 0.82-0.75 (m, 1H), 0.32-0.27 (m, 1H); 485.4 |
| 124 | 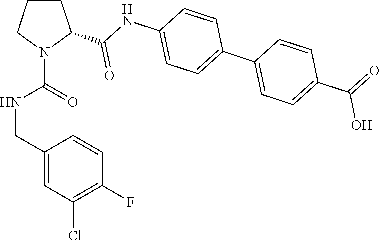 [SMILES: O=C(O)c1ccc(-c2ccc(NC(=O)[C@H]3CCCN3C(=O)NCc3ccc(F)c(Cl)c3)cc2)cc1] | 2.61 minutes¹; 496.2 (chlorine isotope pattern observed) |
| 125 | 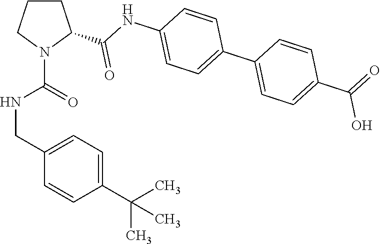 [SMILES: CC(C)(C)c1ccc(CNC(=O)N2CCC[C@@H]2C(=O)Nc2ccc(-c3ccc(C(=O)O)cc3)cc2)cc1] | 2.89 minutes¹; 500.3 |
| 126 | 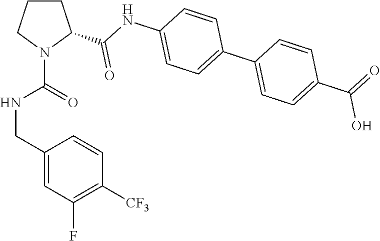 [SMILES: O=C(O)c1ccc(-c2ccc(NC(=O)[C@H]3CCCN3C(=O)NCc3ccc(C(F)(F)F)c(F)c3)cc2)cc1] | 2.69 minutes¹; 530.2 |
| 127 | 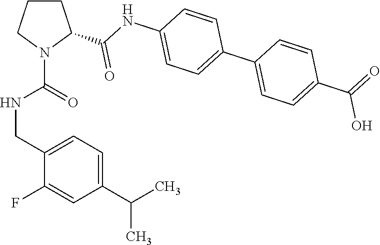 [SMILES: CC(C)c1ccc(CNC(=O)N2CCC[C@@H]2C(=O)Nc2ccc(-c3ccc(C(=O)O)cc3)cc2)c(F)c1] | 3.01 minutes¹; 504.4 |
| 128 | 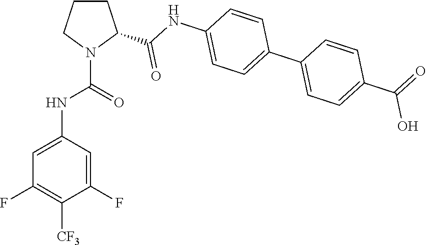 [SMILES: O=C(O)c1ccc(-c2ccc(NC(=O)[C@H]3CCCN3C(=O)Nc3cc(F)c(C(F)(F)F)c(F)c3)cc2)cc1] | 2.89 minutes¹; 534.2 |
| 129 | 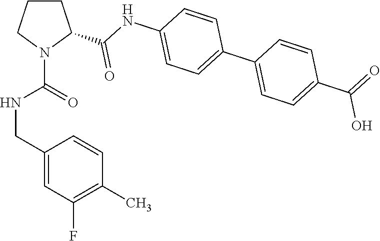 [SMILES: Cc1ccc(CNC(=O)N2CCC[C@@H]2C(=O)Nc2ccc(-c3ccc(C(=O)O)cc3)cc2)cc1F] | 2.60 minutes¹; 476.2 |
| 130 | 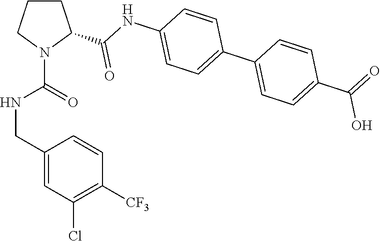 [SMILES: O=C(O)c1ccc(-c2ccc(NC(=O)[C@H]3CCCN3C(=O)NCc3ccc(C(F)(F)F)c(Cl)c3)cc2)cc1] | 2.78 minutes¹; 546.2 (chlorine isotope pattern observed) |
| 131 | 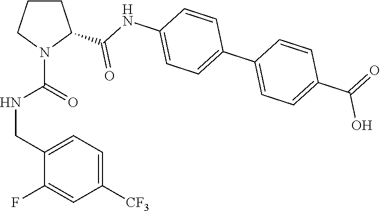 [SMILES: O=C(O)c1ccc(-c2ccc(NC(=O)[C@H]3CCCN3C(=O)NCc3ccc(C(F)(F)F)cc3F)cc2)cc1] | 2.71 minutes¹; 530.2 |
| 132 | 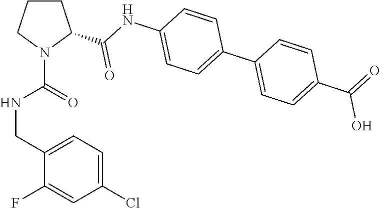 [SMILES: O=C(O)c1ccc(-c2ccc(NC(=O)[C@H]3CCCN3C(=O)NCc3ccc(Cl)cc3F)cc2)cc1] | 2.78 minutes¹; 496.4 (chlorine isotope pattern observed) |
| 133 | 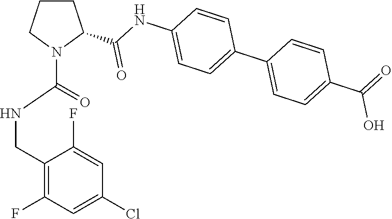 [SMILES: O=C(O)c1ccc(-c2ccc(NC(=O)[C@H]3CCCN3C(=O)NCc3c(F)cc(Cl)cc3F)cc2)cc1] | 2.79 minutes¹; 514.3 (chlorine isotope pattern observed) |
| 134 | 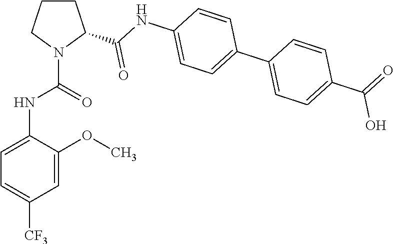 [SMILES: COc1cc(C(F)(F)F)ccc1NC(=O)N1CCC[C@@H]1C(=O)Nc1ccc(-c2ccc(C(=O)O)cc2)cc1] | 3.05 minutes¹; 528.3 |
| 135 | 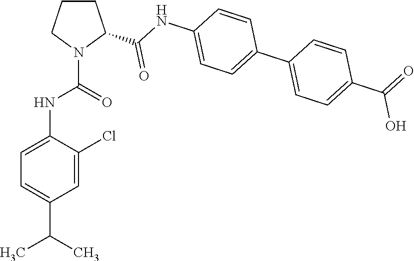 [SMILES: CC(C)c1ccc(NC(=O)N2CCC[C@@H]2C(=O)Nc2ccc(-c3ccc(C(=O)O)cc3)cc2)c(Cl)c1] | 3.21 minutes¹; 506.4 (chlorine isotope pattern observed) |
| 136 | 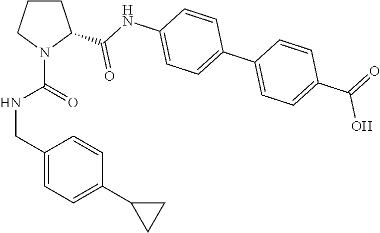 [SMILES: O=C(O)c1ccc(-c2ccc(NC(=O)[C@H]3CCCN3C(=O)NCc3ccc(C4CC4)cc3)cc2)cc1] | 2.83 minutes¹; 484.4 |
| 137 | 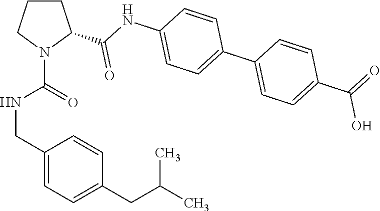 [SMILES: CC(C)Cc1ccc(CNC(=O)N2CCC[C@@H]2C(=O)Nc2ccc(-c3ccc(C(=O)O)cc3)cc2)cc1] | 3.12 minutes¹; 500.4 |
| 138 | 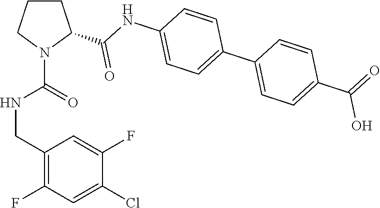 [SMILES: O=C(O)c1ccc(-c2ccc(NC(=O)[C@H]3CCCN3C(=O)NCc3cc(F)c(Cl)cc3F)cc2)cc1] | 2.80 minutes¹; 514.3 (chlorine isotope pattern observed) |
| 139 | 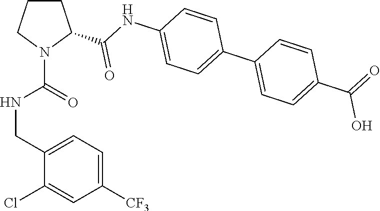 [SMILES: O=C(O)c1ccc(-c2ccc(NC(=O)[C@H]3CCCN3C(=O)NCc3ccc(C(F)(F)F)cc3Cl)cc2)cc1] | 2.97 minutes¹; 546.3 (chlorine isotope pattern observed) |
| 140 | 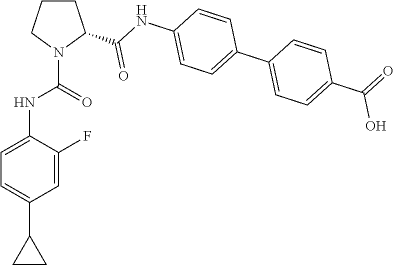 [SMILES: O=C(O)c1ccc(-c2ccc(NC(=O)[C@H]3CCCN3C(=O)Nc3ccc(C4CC4)cc3F)cc2)cc1] | 2.86 minutes¹; 488.4 |
| 141 | 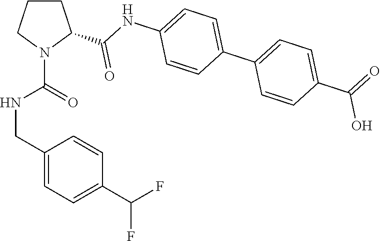 [SMILES: O=C(O)c1ccc(-c2ccc(NC(=O)[C@H]3CCCN3C(=O)NCc3ccc(C(F)F)cc3)cc2)cc1] | 2.65 minutes¹; 494.4 |
| 142 | 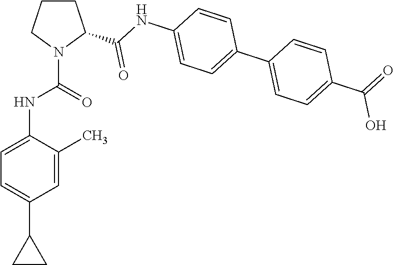 [SMILES: Cc1cc(C2CC2)ccc1NC(=O)N1CCC[C@@H]1C(=O)Nc1ccc(-c2ccc(C(=O)O)cc2)cc1] | 2.87 minutes¹; 484.4 |
| 143 | 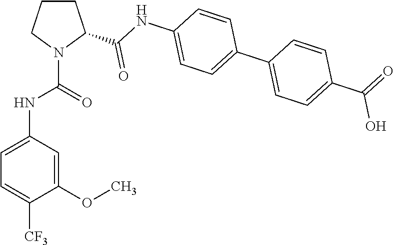 [SMILES: COc1cc(NC(=O)N2CCC[C@@H]2C(=O)Nc2ccc(-c3ccc(C(=O)O)cc3)cc2)ccc1C(F)(F)F] | 2.91 minutes¹; 528.4 |
| 144 | 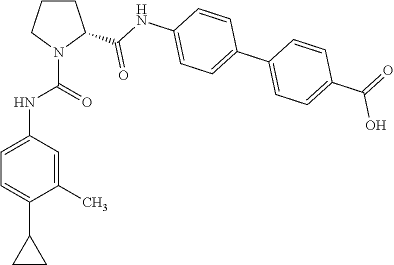 [SMILES: Cc1cc(NC(=O)N2CCC[C@@H]2C(=O)Nc2ccc(-c3ccc(C(=O)O)cc3)cc2)ccc1C1CC1] | 2.97 minutes¹; 484.4 |
| 145 | 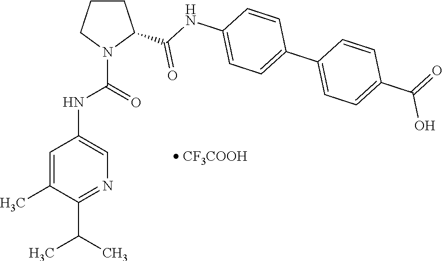 [SMILES: Cc1cc(NC(=O)N2CCC[C@@H]2C(=O)Nc2ccc(-c3ccc(C(=O)O)cc3)cc2)cnc1C(C)C ; "· CF3COOH"] | ¹H NMR, characteristic peaks: δ 10.19 (s, 1H), 8.90 (v br s, 1H), 8.68 (br s, 1H), 8.11 (v br s, 1H), 7.99 (d, J = 8.4 Hz, 2H), 7.78 (d, J = 8.5 Hz, 2H), 7.72 (AB quartet, J_{AB} = 8.8 Hz, Δv_{AB} = 8.9 Hz, 4H), 4.53-4.47 (m, 1H), 3.69-3.63 (m, 1H, assumed; partially obscured by water peak), 3.59-3.52 (m, 1H, assumed; partially obscured by water peak), 2.38 (s, 3H), 2.28- 2.19 (m, 1H), 2.09-1.91 (m, 3H), 1.24 (d, J = 6.9 Hz, 6H); 487.5 |
| 146 | 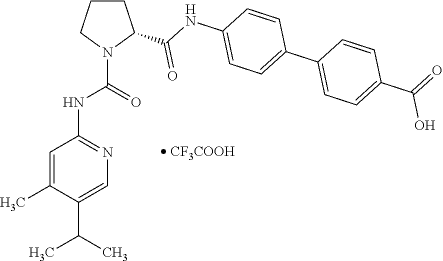 [SMILES: Cc1cc(NC(=O)N2CCC[C@@H]2C(=O)Nc2ccc(-c3ccc(C(=O)O)cc3)cc2)ncc1C(C)C ; "· CF3COOH"] | ¹H NMR, characteristic peaks: δ 10.26 (s, 1H), 8.03 (s, 1H), 7.99 (d, J = 8.4 Hz, 2H), 7.78 (d, J = 8.4 Hz, 2H), 7.75-7.70 (m, 4H), 7.56- 7.51 (m, 1H), 4.57-4.49 (m, 1H), 3.73-3.66 (m, 1H, assumed; partially obscured by water peak), 3.65-3.58 (m, 1H, assumed; partially obscured by water peak), 3.09 (septet, J = 6.9 Hz, 1H), 2.41 (s, 3H), 2.31-2.22 (m, 1H), 2.10-1.93 (m, 3H), 1.19 (d, J = 6.9 Hz, 6H); 487.5 |
| 147 | 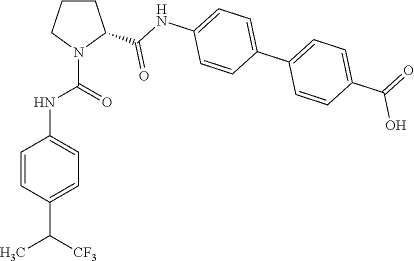 [SMILES: CC(c1ccc(NC(=O)N2CCC[C@@H]2C(=O)Nc2ccc(-c3ccc(C(=O)O)cc3)cc2)cc1)C(F)(F)F] | 10.15 (s, 1H), 8.35 (s, 1H), 7.99 (d, J = 8.4 Hz, 2H), 7.78 (d, J = 8.4 Hz, 2H), 7.72 (AB quartet, J_{AB} = 8.8 Hz, Δv_{AB} = 16.9 Hz, 4H), 7.51 (d, J = 8.3 Hz, 2H), 7.23 (d, J = 8.4 Hz, 2H), 4.48 (dd, J = 8.4, 3.8 Hz, 1H), 3.70-3.62 (m, 2H), 3.56-3.50 (m, 1H), 2.24-2.16 (m, 1H), 2.08-2.00 (m, 1H), 2.00-1.90 (m, 2H), 1.40 (d, J = 7.2 Hz, 3H); 526.4 |
| 148 | 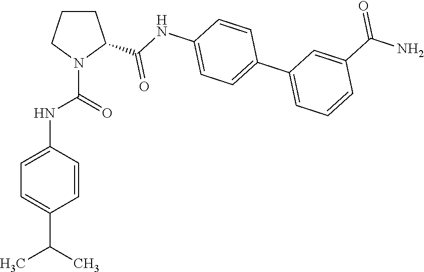 [SMILES: CC(C)c1ccc(NC(=O)N2CCC[C@@H]2C(=O)Nc2ccc(-c3cccc(C(N)=O)c3)cc2)cc1] | 2.66 minutes¹; 471.3 |
| 149 | 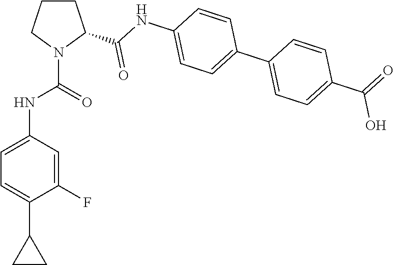 [SMILES: O=C(O)c1ccc(-c2ccc(NC(=O)[C@H]3CCCN3C(=O)Nc3ccc(C4CC4)c(F)c3)cc2)cc1] | ¹H NMR (400 MHz, DMSO-d₆) δ 12.91 (br s, 1H), 10.14 (s, 1H), 8.39 (s, 1H), 7.99 (d, J = 8.5 Hz, 2H), 7.78 (d, J = 8.5 Hz, 2H), 7.72 (AB quartet, J_{AB} = 9.1 Hz, Δv_{AB} = 10.8 Hz, 4H), 7.43 (dd, J = 13.4, 2.1 Hz, 1H), 7.20 (dd, J = 8.6, 2.1 Hz, 1H), 6.84 (t, J = 8.8 Hz, 1H), 4.47 (dd, J = 8.3, 3.7 Hz, 1H), 3.69-3.59 (m, 1H), 3.57-3.46 (m, 1H), 2.26-2.13 (m, 1H), 2.10- 1.86 (m, 4H), 0.93-0.84 (m, 2H), 0.67- 0.59 (m, 2H); 488.3 |
| 150 | 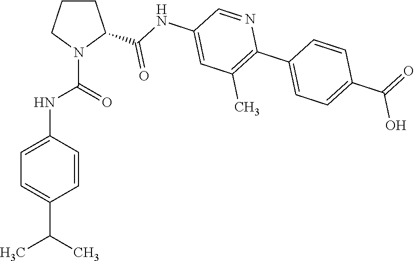 [SMILES: Cc1cc(NC(=O)[C@H]2CCCN2C(=O)Nc2ccc(C(C)C)cc2)cnc1-c1ccc(C(=O)O)cc1] | ¹H NMR, characteristic peaks: δ 10.29 (s, 1H), 8.70 (d, J = 2.4 Hz, 1H), 8.22 (s, 1H), 8.04 (br s, 1H), 8.01 (d, J = 8.4 Hz, 2H), 7.65 (d, J = 8.4 Hz, 2H), 7.39 (d, J = 8.5 Hz, 2H), 7.09 (d, J = 8.8 Hz, 2H), 4.47 (dd, J = 8.4, 3.8 Hz, 1H), 3.67-3.61 (m, 1H, assumed; partially obscured by water peak), 2.80 (septet, J = 6.9 Hz, 1H), 2.32 (s, 3H), 2.25-2.16 (m, 1H), 2.08-1.89 (m, 3H), 1.16 (d, J = 6.9 Hz, 6H); 487.5 |
| 151 | 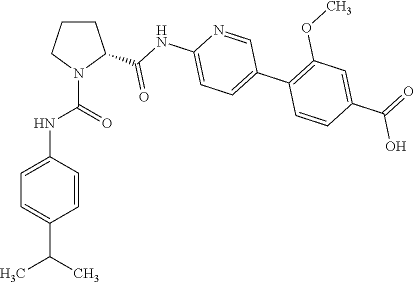 [SMILES: COc1cc(C(=O)O)ccc1-c1ccc(NC(=O)[C@H]2CCCN2C(=O)Nc2ccc(C(C)C)cc2)nc1] | ¹H NMR, characteristic peaks: δ 10.57 (s, 1H), 8.49-8.45 (m, 1H), 8.24-8.20 (m, 1H), 8.12 (d, J = 8.1 Hz, 1H), 7.97-7.93 (m, 1H), 7.65- 7.61 (m, 1H), 7.60 (br s, 1H), 7.50-7.46 (m, 1H), 7.39 (br d, J = 8.7 Hz, 2H), 7.09 (br d, J = 8.7 Hz, 2H), 4.64-4.59 (m, 1H), 3.84 (br s, 3H), 3.68-3.60 (m, 1H), 2.85-2.75 (m, 1H), 2.22-2.13 (m, 1H), 2.06-1.91 (m, 3H), 1.16 (br d, J = 6.9 Hz, 6H); 503.5 |
| 152 | 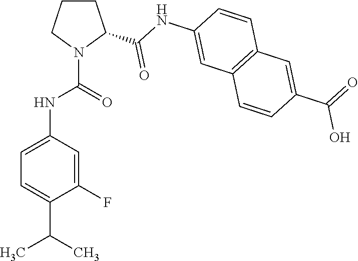 [SMILES: CC(C)c1ccc(NC(=O)N2CCC[C@@H]2C(=O)Nc2ccc3cc(C(=O)O)ccc3c2)cc1F] | ¹H NMR, characteristic peaks: δ 10.36 (s, 1H), 8.50 (s, 1H), 8.43 (s, 1H), 8.38 (s, 1H), 8.05 (d, J = 8.9 Hz, 1H), 7.91 (dd, component of ABX system, J = 8.6, 1.7 Hz, 1H), 7.86 (d, half of AB quartet, J = 8.6 Hz, 1H), 7.68 (br d, J = 9.0 Hz, 1H), 7.42-7.35 (m, 1H), 7.25-7.20 (m, 1H), 7.15 (t, J = 8.6 Hz, 1H), 4.55-4.48 (m, 1H), 3.69-3.61 (m, 1H, assumed; largely obscured by solvent peak), 3.09-3.01 (m, 1H), 2.27-2.18 (m, 1H), 2.09-1.91 (m, 3H), 1.16 (dd, J = 6.9, 1.8 Hz, 6H); 464.4 |
| 153 | 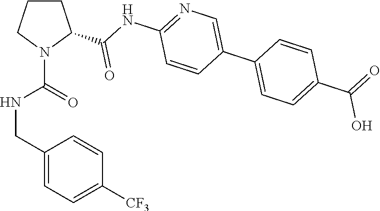 [SMILES: O=C(O)c1ccc(-c2ccc(NC(=O)[C@H]3CCCN3C(=O)NCc3ccc(C(F)(F)F)cc3)nc2)cc1] | ¹H NMR, characteristic peaks: δ 10.60 (s, 1H), 8.74-8.69 (m, 1H), 8.19-8.15 (m, 2H), 8.02 (d, J = 8.5 Hz, 2H), 7.85 (d, J = 8.5 Hz, 2H), 7.64 (d, J = 8.0 Hz, 2H), 7.49 (d, J = 8.0 Hz, 2H), 7.05 (br t, J = 6.0 Hz, 1H), 4.56-4.51 (m, 1H), 4.36 (dd, component of ABX system, J = 16.0, 5.9 Hz, 1H), 4.28 (dd, component of ABX system, J = 16.0, 5.8 Hz, 1H), 2.17- 2.06 (m, 1H), 2.01-1.87 (m, 3H); 513.4 |
| 154 | 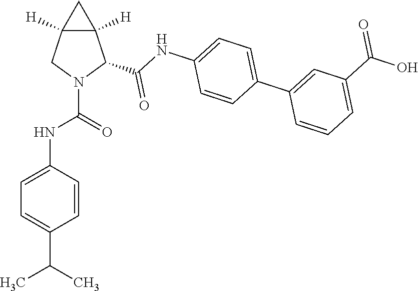 [SMILES: CC(C)c1ccc(NC(=O)N2C[C@H]3C[C@H]3[C@@H]2C(=O)Nc2ccc(-c3cccc(C(=O)O)c3)cc2)cc1] | 10.31 (s, 1H), 8.16 (br s, 2H), 7.92-7.87 (m, 2H), 7.75 (d, J = 8.2 Hz, 2H), 7.67 (d, J = 8.3 Hz, 2H), 7.57 (t, J = 7.7 Hz, 1H), 7.36 (d, J = 8.1 Hz, 2H), 7.07 (d, J = 8.3 Hz, 2H), 4.55 (s, 1H), 3.72 (d, half of AB quartet, J = 9.4 Hz, 1H), 3.69 (dd, component of ABX system, J = 9.4, 3.6 Hz, 1H), 2.79 (septet, J = 6.8 Hz, 1H), 1.76-1.69 (m, 1H), 1.68-1.92 (m, 1H), 1.15 (d, J = 6.9 Hz, 6H), 0.82-0.76 (m, 1H), 0.33- 0.27 (m, 1H); 484.5 |
| 155 | 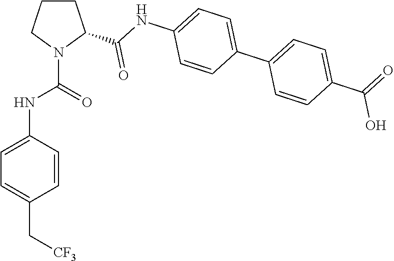 [SMILES: O=C(O)c1ccc(-c2ccc(NC(=O)[C@H]3CCCN3C(=O)Nc3ccc(CC(F)(F)F)cc3)cc2)cc1] | 12.92 (br s, 1H), 10.15 (s, 1H), 8.35 (s, 1H), 7.99 (d, J = 8.0 Hz, 2H), 7.78 (d, J = 8.1 Hz, 2H), 7.72 (AB quartet, J_{AB} = 8.6 Hz, Δv_{AB} = 17.6 Hz, 4H), 7.51 (d, J = 8.2 Hz, 2H), 7.20 (d, J = 8.2 Hz, 2H), 4.48 (dd, J = 8.4, 3.8 Hz, 1H), 3.70-3.62 (m, 1H), 3.57-3.47 (m, 3H), 2.25- 2.15 (m, 1H), 2.09-1.89 (m, 3H); 512.4 |
| 156 | 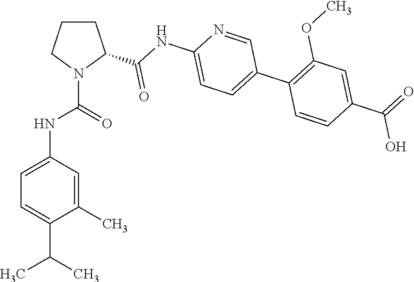 [SMILES: COc1cc(C(=O)O)ccc1-c1ccc(NC(=O)[C@H]2CCCN2C(=O)Nc2ccc(C(C)C)c(C)c2)nc1] | ¹H NMR, characteristic peaks: δ 10.56 (s, 1H), 8.46 (d, J = 2.4 Hz, 1H), 8.13 (s, 1H), 8.11 (d, J = 9.0 Hz, 1H), 7.95 (dd, J = 8.7, 2.5 Hz, 1H), 7.63 (br d, J = 7.7 Hz, 1H), 7.60 (s, 1H), 7.48 (d, J = 7.8 Hz, 1H), 7.28-7.23 (m, 2H), 7.07 (d, J = 8.2 Hz, 1H), 4.63-4.57 (m, 1H), 3.84 (s, 3H), 3.66-3.59 (m, 1H, assumed; partially obscured by water peak), 3.05-2.97 (m, 1H), 2.22 (s, 3H), 2.20-2.12 (m, 1H), 2.04-1.91 (m, 3H), 1.13 (d, J = 6.8 Hz, 6H); 517.5 |
| 157 | 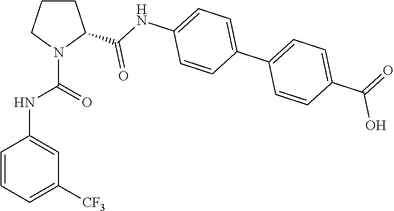 [SMILES: O=C(O)c1ccc(-c2ccc(NC(=O)[C@H]3CCCN3C(=O)Nc3cccc(C(F)(F)F)c3)cc2)cc1] | ¹H NMR, characteristic peaks: δ 10.08 (s, 1H), 7.99 (d, J = 8.1 Hz, 2H), 7.78 (d, J = 8.1 Hz, 2H), 7.74-7.67 (m, 4H), 7.65 (d, J = 8.0 Hz, 2H), 7.49 (d, J = 8.0 Hz, 2H), 7.02 (t, J = 6.1 Hz, 1H), 4.42-4.32 (m, 2H), 4.28 (dd, component of ABX system, J = 16.1, 6.0 Hz, 1H), 3.56-3.48 (m, 1H), 2.19-2.08 (m, 1H), 2.04-1.87 (m, 3H); 512.3 |
| 158 | 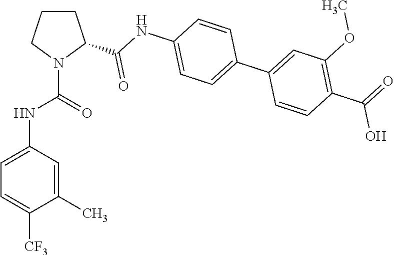 [SMILES: COc1cc(-c2ccc(NC(=O)[C@H]3CCCN3C(=O)Nc3ccc(C(F)(F)F)c(C)c3)cc2)ccc1C(=O)O] | ¹H NMR (400 MHz, DMSO-d₆) δ 10.18 (s, 1H), 8.62 (s, 1H), 7.73 (s, 4H), 7.71 (d, J = 8.0 Hz, 1H), 7.61 br (s, 1H), 7.53 (AB quartet, downfield doublet is broadened, J_{AB} = 8.9 Hz, Δv_{AB} = 17.9 Hz, 2H), 7.32 (d, half of AB quartet, J = 1.6 Hz, 1H), 7.27 (dd, component of ABX system, J = 8.0, 1.6 Hz, 1H), 4.50 (dd, J = 8.3, 3.8 Hz, 1H), 3.91 (s, 3H), 3.72-3.63 (m, 1H), 3.60-3.51 (m, 1H), 2.36 (br s, 3H), 2.28-2.16 (m, 1H), 2.11-1.88 (m, 3H); 542.3 |
| 159 | 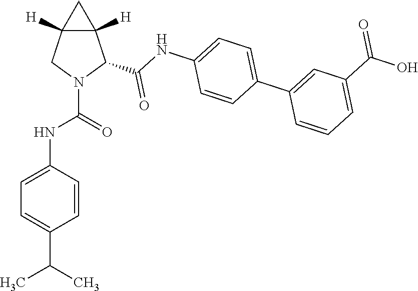 [SMILES: CC(C)c1ccc(NC(=O)N2C[C@@H]3C[C@@H]3[C@@H]2C(=O)Nc2ccc(-c3cccc(C(=O)O)c3)cc2)cc1] | 10.02 (s, 1H), 8.17-8.15 (m, 1H), 8.15 (s, 1H), 7.92-7.87 (m, 2H), 7.70 (AB quartet, J_{AB} = 8.7 Hz, Δv_{AB} = 43.1 Hz, 4H), 7.57 (t, J = 7.7 Hz, 1H), 7.36 (d, J = 8.5 Hz, 2H), 7.07 (d, J = 8.6 Hz, 2H), 4.49 (d, J = 5.2 Hz, 1H), 3.82 (d, J = 9.3 Hz, 1H), 3.58 (dd, J = 9.3, 5.1 Hz, 1H), 2.79 (septet, J = 6.9 Hz, 1H), 1.96-1.90 (m, 1H), 1.80-1.73 (m, 1H), 1.15 (d, J = 6.9 Hz, 6H), 0.82-0.77 (m, 1H), 0.66-0.59 (m, 1H); 484.4 |
| 160 | 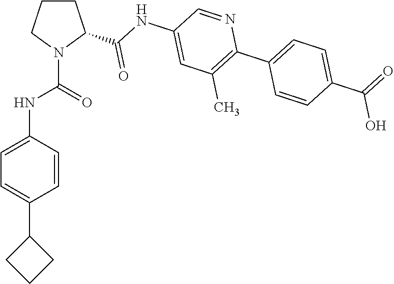 [SMILES: Cc1cc(NC(=O)[C@H]2CCCN2C(=O)Nc2ccc(C3CCC3)cc2)cnc1-c1ccc(C(=O)O)cc1] | ¹H NMR, characteristic peaks: δ 10.30 (s, 1H), 8.71 (s, 1H), 8.23 (s, 1H), 8.05 (s, 1H), 8.01 (d, J = 8.0 Hz, 2H), 7.66 (d, J = 8.0 Hz, 2H), 7.42 (d, J = 8.3 Hz, 2H), 7.09 (d, J = 8.3 Hz, 2H), 4.47 (dd, J = 8.3, 3.7 Hz, 1H), 3.68-3.61 (m, 1H), 3.55-3.49 (m, 1H, assumed; partially obscured by water peak), 2.33 (s, 3H), 2.27-2.17 (m, 3H), 2.09-1.87 (m, 6H), 1.82-1.73 (m, 1H); 499.6 |
| 161 | 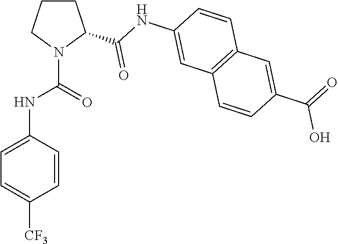 [SMILES: O=C(O)c1ccc2cc(NC(=O)[C@H]3CCCN3C(=O)Nc3ccc(C(F)(F)F)cc3)ccc2c1] | 10.38 (s, 1H), 8.72 (s, 1H), 8.50 (s, 1H), 8.38 (s, 1H), 8.05 (d, J = 8.9 Hz, 1H), 7.89 (AB quartet, J_{AB} = 8.7 Hz, Δv_{AB} = 32.1 Hz, 2H), 7.75 (d, J = 8.6 Hz, 2H), 7.68 (br d, J = 8.9 Hz, 1H), 7.57 (d, J = 8.5 Hz, 2H), 4.54 (dd, J = 8.4, 3.8 Hz, 1H), 3.74-3.66 (m, 1H), 3.62-3.55 (m, 1H, assumed; partially obscured by water peak), 2.29-2.20 (m, 1H), 2.11-1.92 (m, 3H); 472.2 |
| 162 | 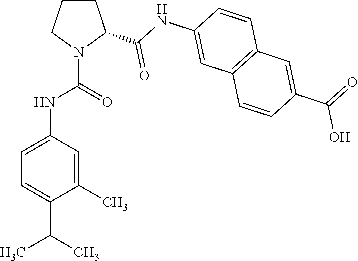 [SMILES: Cc1cc(NC(=O)N2CCC[C@@H]2C(=O)Nc2ccc3cc(C(=O)O)ccc3c2)ccc1C(C)C] | 10.34 (s, 1H), 8.50 (s, 1H), 8.39 (s, 1H), 8.13 (s, 1H), 8.05 (d, J = 8.9 Hz, 1H), 7.89 (AB quartet, J_{AB} = 8.6 Hz, Δv_{AB} = 33.0 Hz, 2H), 7.68 (d, J = 8.8 Hz, 1H), 7.30-7.23 (m, 2H), 7.06 (d, J = 8.2 Hz, 1H), 4.50 (dd, J = 8.4, 3.6 Hz, 1H), 3.69-3.61 (m, 1H), 3.56-3.47 (m, 1H, assumed; partially obscured by water peak), 3.00 (septet, J = 6.9 Hz, 1H), 2.25- 2.16 (m, 1H), 2.21 (s, 3H), 2.10-2.00 (m, 1H), 2.00-1.91 (m, 2H), 1.12 (d, J = 6.8 Hz, 6H); 460.3 |
| 163 | 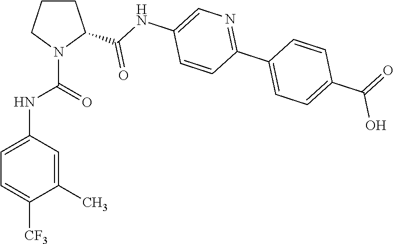 [SMILES: Cc1cc(NC(=O)N2CCC[C@@H]2C(=O)Nc2ccc(-c3ccc(C(=O)O)cc3)nc2)ccc1C(F)(F)F] | ¹H NMR (400 MHz, DMSO-d₆) δ 10.43 (s, 1H), 8.88 (d, J = 2.5 Hz, 1H), 8.64 (s, 1H), 8.20 (dd, J = 8.8, 2.6 Hz, 1H), 8.15 (d, J = 8.6 Hz, 2H), 8.05-7.99 (m, 3H), 7.61 (br s, 1H), 7.53 (AB quartet, downfield doublet is broadened, J_{AB} = 8.9 Hz, Δv_{AB} = 16.9 Hz, 2H), 4.51 (dd, J = 8.6, 3.6 Hz, 1H), 3.72-3.63 (m, 1H), 3.61- 3.52 (m, 1H), 2.38-2.34 (m, 3H), 2.29-2.18 (m, 1H), 2.09-1.91 (m, 3H); 513.3 |
| 164 | 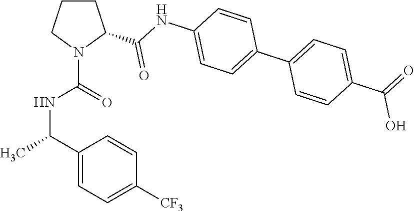 [SMILES: C[C@H](NC(=O)N1CCC[C@@H]1C(=O)Nc1ccc(-c2ccc(C(=O)O)cc2)cc1)c1ccc(C(F)(F)F)cc1] | ¹H NMR (400 MHz, DMSO-d₆) δ 10.04 (s, 1H), 7.98 (d, J = 8.5 Hz, 2H), 7.75 (d, J = 8.5 Hz, 2H), 7.69-7.61 (m, 6H), 7.58 (d, half of AB quartet, J = 8.1 Hz, 2H), 6.71 (d, J = 8.0 Hz, 1H), 4.95-4.85 (m, 1H), 4.44-4.37 (m, 1H), 3.61-3.52 (m, 1H), 3.42-3.35 (m, 1H, assumed; partially obscured by water peak), 2.16-2.04 (m, 1H), 2.04-1.87 (m, 3H), 1.40 (d, J = 7.1 Hz, 3H); 526.3 |
| 165 | 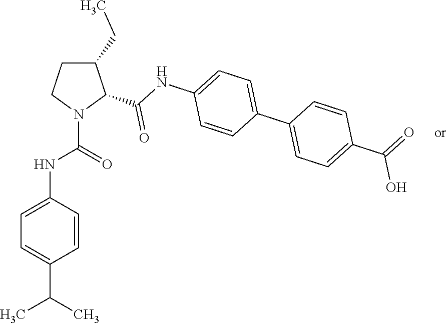 [SMILES: CC[C@H]1CCN(C(=O)Nc2ccc(C(C)C)cc2)[C@H]1C(=O)Nc1ccc(-c2ccc(C(=O)O)cc2)cc1] | ¹H NMR (400 MHz, DMSO-d₆) δ 10.22 (s, 1H), 8.13 (s, 1H), 7.99 (d, J = 8.4 Hz, 2H), 7.77 (d, J = 8.4 Hz, 2H), 7.72 (AB quartet, J_{AB} = 8.7 Hz, Δv_{AB} = 20.7 Hz, 4H), 7.40 (d, J = 8.5 Hz, 2H), 7.08 (d, J = 8.6 Hz, 2H), 4.49 (d, J = 8.0 Hz, 1H), 3.75 (t, J = 9.0 Hz, 1H), 3.51-3.39 (m, 1H, assumed; partially obscured by water peak), 2.80 (septet, J = 6.9 Hz, 1H), 2.35- 2.21 (m, 1H), 2.16-2.05 (m, 1H), 1.92-1.77 (m, 1H), 1.65-1.51 (m, 1H), 1.20-1.05 (m, 1H), 1.16 (d, J = 6.9 Hz, 6H), 0.94 (t, J = 7.3 Hz, 3H); 500.4 |
| | 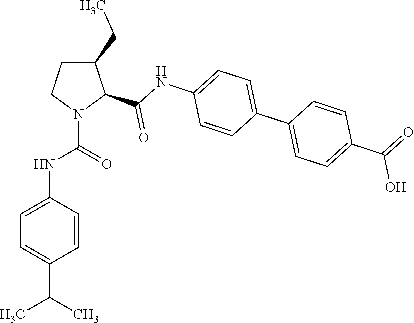 [SMILES: CC[C@@H]1CCN(C(=O)Nc2ccc(C(C)C)cc2)[C@@H]1C(=O)Nc1ccc(-c2ccc(C(=O)O)cc2)cc1] | |
| | From DIAST-1 of precursor (see footnote 27 in Table 2 | |
| 166 | 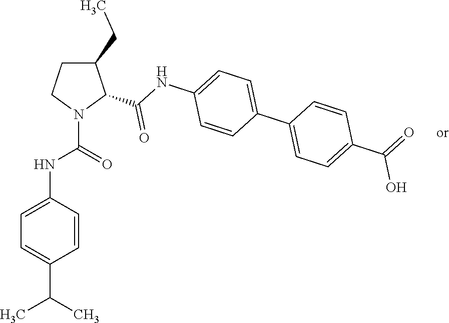 [SMILES: CC[C@@H]1CCN(C(=O)Nc2ccc(C(C)C)cc2)[C@H]1C(=O)Nc1ccc(-c2ccc(C(=O)O)cc2)cc1] | ¹H NMR (400 MHz, DMSO-d₆) δ 10.17 (s, 1H), 8.18 (s, 1H), 7.99 (d, J = 8.4 Hz, 2H), 7.78 (d, J = 8.4 Hz, 2H), 7.73 (AB quartet, J_{AB} = 8.8 Hz, Δv_{AB} = 20.8 Hz, 4H), 7.38 (d, J = 8.6 Hz, 2H), 7.08 (d, J = 8.5 Hz, 2H), 4.10 (d, J = 5.3 Hz, 1H), 3.65-3.56 (m, 2H), 2.86-2.73 (m, 1H), 2.22-2.05 (m, 2H), 1.73-1.56 (m, 2H), 1.46-1.33 (m, 1H), 1.15 (d, J = 6.9 Hz, 6H), 0.95 (t, J = 7.4 Hz, 3H); 500.1 |
| | 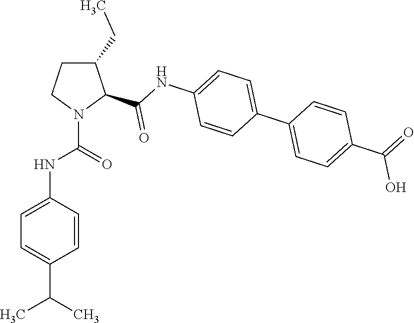 [SMILES: CC[C@H]1CCN(C(=O)Nc2ccc(C(C)C)cc2)[C@@H]1C(=O)Nc1ccc(-c2ccc(C(=O)O)cc2)cc1] | |
| | From DIAST-2 of precursor (see footnote 27 in Table 2 | |
| 167 | 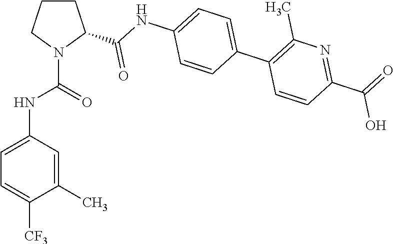 [SMILES: Cc1cc(NC(=O)N2CCC[C@@H]2C(=O)Nc2ccc(-c3ccc(C(=O)O)nc3C)cc2)ccc1C(F)(F)F] | 10.18 (s, 1H), 8.61 (s, 1H), 7.93 (d, J = 7.8 Hz, 1H), 7.77 (d, J = 7.9 Hz, 1H), 7.74 (d, J = 8.6 Hz, 2H), 7.61 (br s, 1H), 7.53 (AB quartet, downfield doublet is broadened, J_{AB} = 8.8 Hz, Δv_{AB} = 25.7 Hz, 2H), 7.40 (d, J = 8.7 Hz, 2H), 4.49 (dd, J = 8.3, 3.9 Hz, 1H), 3.71-3.64 (m, 1H, assumed; partially obscured by water peak), 3.59-3.53 (m, 1H, assumed; partially obscured by water peak), 2.51 (s, 3H), 2.36 (br s, 3H), 2.27- 2.18 (m, 1H), 2.09-2.00 (m, 1H), 2.00-1.91 (m, 2H); 527.4 |
| 168 | 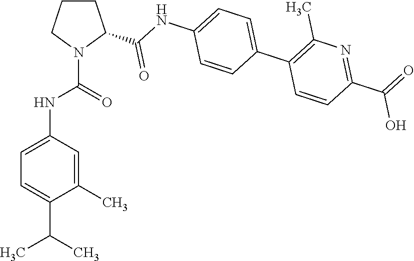 [SMILES: Cc1cc(NC(=O)N2CCC[C@@H]2C(=O)Nc2ccc(-c3ccc(C(=O)O)nc3C)cc2)ccc1C(C)C] | 10.15 (s, 1H), 8.11 (s, 1H), 7.92 (d, J = 7.9 Hz, 1H), 7.76 (d, J = 7.9 Hz, 1H), 7.74 (d, J = 8.6 Hz, 2H), 7.39 (d, J = 8.6 Hz, 2H), 7.29-7.25 (m, 2H), 7.07 (d, J = 8.2 H, 1H), 4.46 (dd, J = 8.2, 3.8 Hz, 1H), 3.67-3.61 (m, 1H), 3.53- 3.47 (m, 1H, assumed; partially obscured by water peak), 3.05-2.97 (m, 1H), 2.50 (s, 3H, assumed; partially obscured by solvent peak), 2.22 (s, 3H), 2.21-2.15 (m, 1H), 2.08-1.99 (m, 1H), 1.99-1.89 (m, 2H), 1.13 (d, J = 6.8 Hz, 6H); 501.5 |
| 169 | 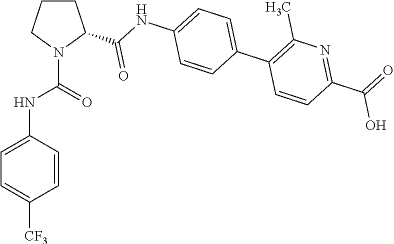 [SMILES: Cc1nc(C(=O)O)ccc1-c1ccc(NC(=O)[C@H]2CCCN2C(=O)Nc2ccc(C(F)(F)F)cc2)cc1] | 10.19 (s, 1H), 8.70 (s, 1H), 7.93 (d, J = 7.9 Hz, 1H), 7.78-7.72 (m, 5H), 7.58 (d, J = 8.6 Hz, 2H), 7.40 (d, J = 8.6 Hz, 2H), 4.50 (dd, J = 8.3, 3.9 Hz, 1H), 3.72-3.66 (m, 1H), 3.60-3.54 (m, 1H, assumed; partially obscured by water peak), 2.51 (s, 3H, assumed; partially obscured by solvent peak), 2.27-2.18 (m, 1H), 2.10-1.88 (m, 3H); 513.2 |
| 170 | 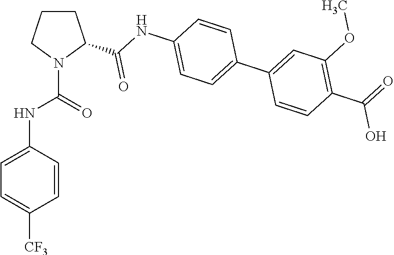 [SMILES: COc1cc(-c2ccc(NC(=O)[C@H]3CCCN3C(=O)Nc3ccc(C(F)(F)F)cc3)cc2)ccc1C(=O)O] | ¹H NMR (400 MHz, DMSO-d₆) δ 10.18 (s, 1H), 8.71 (s, 1H), 7.76 (d, J = 8.7 Hz, 2H), 7.73 (s, 4H), 7.71 (d, J = 8.0 Hz, 1H), 7.58 (d, J = 8.6 Hz, 2H), 7.32 (d, half of AB quartet, J = 1.6 Hz, 1H), 7.27 (dd, component of ABX system, J = 8.1, 1.6 Hz, 1H), 4.51 (dd, J = 8.3, 3.8 Hz, 1H), 3.91 (s, 3H), 3.74-3.65 (m, 1H), 3.61- 3.53 (m, 1H), 2.29-2.17 (m, 1H), 2.11-1.89 (m, 3H); 528.3 |
| 171 | 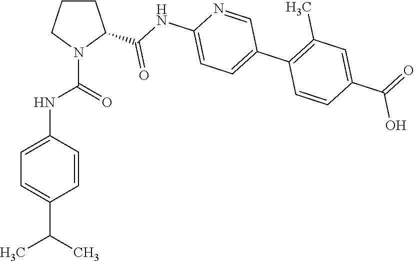 [SMILES: Cc1cc(C(=O)O)ccc1-c1ccc(NC(=O)[C@H]2CCCN2C(=O)Nc2ccc(C(C)C)cc2)nc1] | ¹H NMR, characteristic peaks: δ 10.61 (s, 1H), 8.33 (d, J = 2.4 Hz, 1H), 8.22 (s, 1H), 8.15 (d, J = 8.6 Hz, 1H), 7.89 (s, 1H), 7.86-7.81 (m, 2H), 7.39 (br d, J = 8 Hz, 2H), 7.37 (d, J = 8.2 Hz, 1H), 7.09 (d, J = 8.4 Hz, 2H), 4.64-4.59 (m, 1H), 3.67-3.60 (m, 1H, assumed; partially obscured by water peak), 2.80 (septet, J = 6.9 Hz, 1H), 2.31 (s, 3H), 2.21- 2.13 (m, 1H), 2.05-1.91 (m, 3H), 1.16 (d, J = 6.9 Hz, 6H); 487.4 |
| 172 | 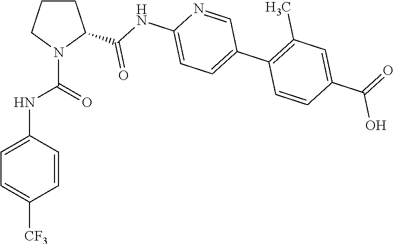 [SMILES: Cc1cc(C(=O)O)ccc1-c1ccc(NC(=O)[C@H]2CCCN2C(=O)Nc2ccc(C(F)(F)F)cc2)nc1] | 10.67 (s, 1H), 8.71 (s, 1H), 8.34 (d, J = 2.4 Hz, 1H), 8.15 (d, J = 8.6 Hz, 1H), 7.89 (br s, 1H), 7.86-7.82 (m, 2H), 7.74 (d, J = 8.5 Hz, 2H), 7.58 (d, J = 8.6 Hz, 2H), 7.37 (d, J = 7.9 Hz, 1H), 4.68-4.60 (m, 1H), 3.71-3.64 (m, 1H, assumed; partially obscured by water peak), 3.60-3.54 (m, 1H, assumed; partially obscured by water peak), 2.31 (s, 3H), 2.26- 2.18 (m, 1H), 2.07-1.91 (m, 3H); 513.4 |
| 173 | 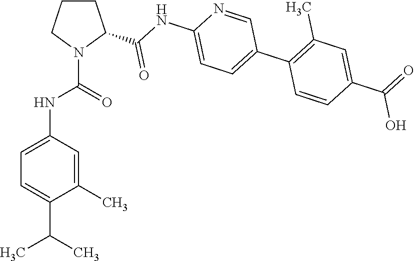 [SMILES: Cc1cc(C(=O)O)ccc1-c1ccc(NC(=O)[C@H]2CCCN2C(=O)Nc2ccc(C(C)C)c(C)c2)nc1] | ¹H NMR, characteristic peaks: δ 10.61 (s, 1H), 8.33 (d, J = 2.5 Hz, 1H), 8.17-8.12 (m, 2H), 7.89 (br s, 1H), 7.86-7.81 (m, 2H), 7.37 (d, J = 7.9 Hz, 1H), 7.28-7.23 (m, 2H), 7.07 (d, J = 8.2 Hz, 1H), 4.64-4.57 (m, 1H), 3.66- 3.59 (m, 1H, assumed; partially obscured by water peak), 3.01 (septet, J = 6.8 Hz, 1H), 2.31 (s, 3H), 2.22 (s, 3H), 2.21-2.13 (m, 1H), 2.05-1.91 (m, 3H), 1.13 (d, J = 6.8 Hz, 6H); 501.4 |
| 174 | 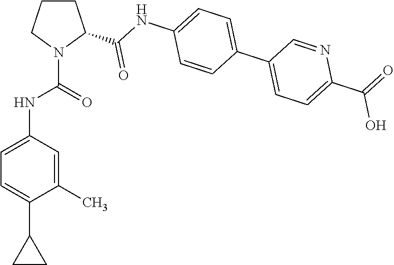 [SMILES: Cc1cc(NC(=O)N2CCC[C@@H]2C(=O)Nc2ccc(-c3ccc(C(=O)O)nc3)cc2)ccc1C1CC1] | ¹H NMR, characteristic peaks: δ 10.19 (s, 1H), 9.00 (br s, 1H), 8.24 (dd, J = 8.2, 2.4 Hz, 1H), 8.13 (s, 1H), 8.09 (dd, J = 8.2, 0.8 Hz, 1H), 7.78 (AB quartet, J_{AB} = 8.8 Hz, Δv_{AB} = 10.8 Hz, 4H), 7.29 (br s, 1H), 7.21 (br d, J = 8.4 Hz, 1H), 6.79 (d, J = 8.4 Hz, 1H), 4.46 (dd, J = 8.3, 3.8 Hz, 1H), 3.65-3.59 (m, 1H, assumed; partially obscured by water peak), 2.30 (s, 3H), 2.23-2.14 (m, 1H), 2.07-1.98 (m, 1H), 1.98-1.88 (m, 2H), 1.83-1.76 (m, 1H), 0.86- 0.80 (m, 2H), 0.53-0.47 (m, 2H); 485.4 |
| 175 | 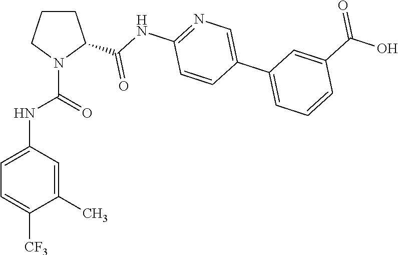 [SMILES: Cc1cc(NC(=O)N2CCC[C@@H]2C(=O)Nc2ccc(-c3cccc(C(=O)O)c3)cn2)ccc1C(F)(F)F] | ¹H NMR (400 MHz, DMSO-d₆) δ 10.69 (s, 1H), 8.69 (dd, J = 2.2, 1.1 Hz, 1H), 8.62 (s, 1H), 8.20 (t, J = 1.8 Hz, 1H), 8.18 (d, half of AB quartet, J = 8.8 Hz, 1H), 8.14 (dd, component of ABX system, J = 8.8, 2.4 Hz, 1H), 7.98- 7.93 (m, 2H), 7.63-7.58 (m, 2H), 7.53 (AB quartet, downfield doublet is broadened, J_{AB} = 8.8 Hz, Δv_{AB} = 14.4 Hz, 2H), 4.69-4.59 (m, 1H), 3.71-3.62 (m, 1H), 3.61-3.52 (m, 1H), 2.36 (br s, 3H), 2.27-2.15 (m, 1H), 2.08- 1.89 (m, 3H); 513.3 |
| 176 | 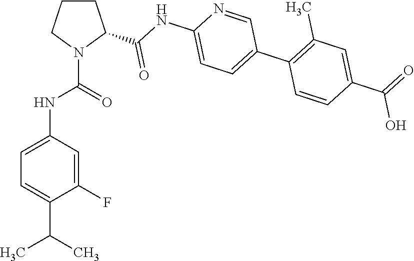 [SMILES: Cc1cc(C(=O)O)ccc1-c1ccc(NC(=O)[C@H]2CCCN2C(=O)Nc2ccc(C(C)C)c(F)c2)nc1] | ¹H NMR, characteristic peaks: δ 10.63 (s, 1H), 8.42 (s, 1H), 8.35-8.32 (m, 1H), 8.15 (d, J = 8.6 Hz, 1H), 7.89 (s, 1H), 7.86-7.81 (m, 2H), 7.42-7.35 (m, 2H), 7.25-7.21 (m, 1H), 7.16 (t, J = 8.6 Hz, 1H), 4.65-4.58 (m, 1H), 3.67- 3.59 (m, 1H, assumed; partially obscured by water peak), 3.10-3.01 (m, 1H), 2.31 (s, 3H), 2.23-2.13 (m, 1H), 2.06-1.91 (m, 3H), 1.17 (d, J = 6.9 Hz, 6H); 505.5 |
| 177 | 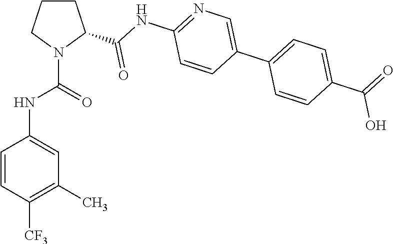 [SMILES: Cc1cc(NC(=O)N2CCC[C@@H]2C(=O)Nc2ccc(-c3ccc(C(=O)O)cc3)cn2)ccc1C(F)(F)F] | 10.71 (s, 1H), 8.73 (d, J = 1.9 Hz, 1H), 8.62 (s, 1H), 8.20-8.15 (m, 2H), 8.02 (d, J = 8.4 Hz, 2H), 7.86 (d, J = 8.4 Hz, 2H), 7.59 (br s, 1H), 7.52 (AB quartet, downfield doublet is broadened, J_{AB} = 8.8 Hz, Δv_{AB} = 17.3 Hz, 2H), 4.67-4.59 (m, 1H), 3.69-3.62 (m, 1H), 3.59- 3.52 (m, 1H, assumed; partially obscured by water peak), 2.36 (s, 3H), 2.26-2.17 (m, 1H), 2.06-1.89 (m, 3H); 513.4 |
| 178 | 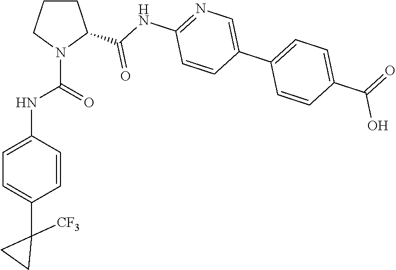 [SMILES: O=C(O)c1ccc(-c2ccc(NC(=O)[C@H]3CCCN3C(=O)Nc3ccc(C4(C(F)(F)F)CC4)cc3)nc2)cc1] | ¹H NMR, characteristic peaks: δ 10.67 (s, 1H), 8.73 (d, J = 1.9 Hz, 1H), 8.41 (s, 1H), 8.20- 8.15 (m, 2H), 8.02 (d, J = 8.3 H, 2H), 7.85 (d, J = 8.1 Hz, 2H), 7.52-7.47 (m, 2H), 7.30 (d, J = 8.3 Hz, 2H), 4.66-4.59 (m, 1H), 3.69- 3.60 (m, 1H, assumed; partially obscured by water peak), 2.24-2.14 (m, 1H), 2.06-1.90 (m, 3H), 1.30-1.24 (m, 2H), 1.07-1.01 (m, 2H); 539.4 |
| 179 | 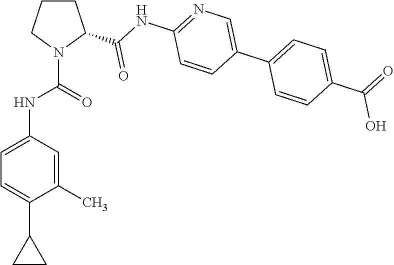 [SMILES: Cc1cc(NC(=O)N2CCC[C@@H]2C(=O)Nc2ccc(-c3ccc(C(=O)O)cc3)cn2)ccc1C1CC1] | 10.65 (s, 1H), 8.74-8.71 (m, 1H), 8.20-8.13 (m, 3H), 8.02 (d, J = 8.4 Hz, 2H), 7.85 (d, J = 8.4 Hz, 2H), 7.29 (br s, 1H), 7.20 (dd, J = 8.4, 2.3 Hz, 1H), 6.80 (d, J = 8.4 Hz, 1H), 4.60 (dd, J = 8.7, 3.5 Hz, 1H), 3.65-3.58 (m, 1H), 3.53- 3.46 (m, 1H, assumed; partially obscured by water peak), 2.30 (s, 3H), 2.21-2.12 (m, 1H), 2.05-1.89 (m, 3H), 1.83-1.77 (m, 1H), 0.86- 0.80 (m, 2H), 0.53-0.48 (m, 2H); 485.4 |
| 180 | 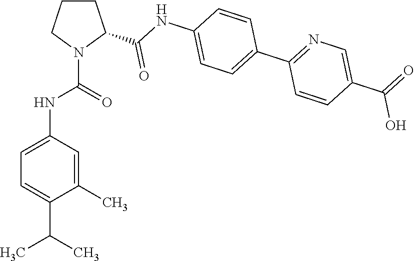 [SMILES: Cc1cc(NC(=O)N2CCC[C@@H]2C(=O)Nc2ccc(-c3ccc(C(=O)O)cn3)cc2)ccc1C(C)C] | ¹H NMR, characteristic peaks: δ 10.22 (s, 1H), 9.12-9.07 (m, 1H), 8.28 (dd, J = 8.4, 2.3 Hz, 1H), 8.16-8.10 (m, 3H), 8.05 (d, J = 8.4 Hz, 1H), 7.76 (d, J = 8.4 Hz, 2H), 7.29-7.23 (m, 2H), 7.06 (d, J = 8.2 Hz, 1H), 4.46 (dd, J = 8.3, 3.7 Hz, 1H), 3.67-3.59 (m, 1H, assumed; partially obscured by water peak), 3.05-2.95 (m, 1H), 2.23-2.14 (m, 1H), 2.22 (s, 3H), 2.07-1.98 (m, 1H), 1.98-1.89 (m, 2H), 1.13 (d, J = 6.9 Hz, 6H); 487.5 |
| 181 | 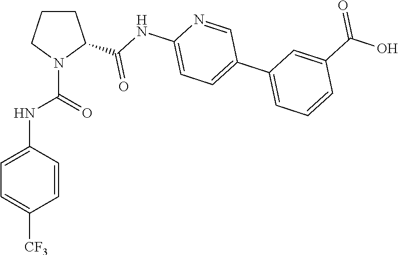 [SMILES: O=C(O)c1cccc(-c2ccc(NC(=O)[C@H]3CCCN3C(=O)Nc3ccc(C(F)(F)F)cc3)nc2)c1] | 10.69 (s, 1H), 8.71 (s, 1H), 8.69 (br s, 1H), 8.21-8.18 (m, 1H), 8.17 (d, half of AB quartet, J = 8.7 Hz, 1H), 8.15 (dd, component of ABX system, J = 8.7, 2.3 Hz, 1H), 7.99- 7.93 (m, 2H), 7.74 (br d, J = 8.3 Hz, 2H), 7.61 (t, J = 7.8 Hz, 1H), 7.58 (d, J = 8.5 Hz, 2H), 4.67-4.61 (m, 1H), 3.71-3.64 (m, 1H, assumed; partially obscured by water peak), 3.61-3.53 (m, 1H, assumed; partially obscured by water peak), 2.26-2.18 (m, 1H), 2.07-1.91 (m, 3H); 499.4 |
| 182 | 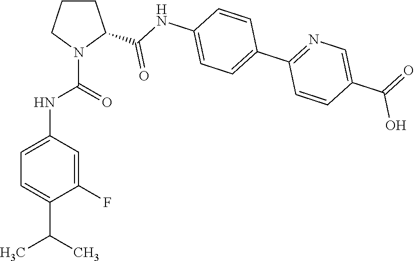 [SMILES: CC(C)c1ccc(NC(=O)N2CCC[C@@H]2C(=O)Nc2ccc(-c3ccc(C(=O)O)cn3)cc2)cc1F] | ¹H NMR, characteristic peaks: δ 10.23 (s, 1H), 9.11-9.07 (m, 1H), 8.41 (s, 1H), 8.28 (dd, J = 8.4, 2.3 Hz, 1H), 8.13 (d, J = 8.4 Hz, 2H), 8.06 (d, J = 8.4 Hz, 1H), 7.76 (d, J = 8.4 Hz, 2H), 7.39 (br d, J = 13.6 Hz, 1H), 7.23 (br d, half of AB quarter, J = 8.5 Hz, 1H), 7.15 (dd, component of ABX system, J = 8.7, 8.7 Hz, 1H), 4.47 (dd, J = 8.4, 3.9 Hz, 1H), 3.67-3.60 (m, 1H, assumed; partially obscured by water peak), 3.06 (septet, J = 6.9 Hz, 1H), 2.25- 2.16 (m, 1H), 2.07-1.99 (m, 1H), 1.99-1.89 (m, 2H), 1.16 (d, J = 6.9 Hz, 6H); 491.5 |
| 183 | 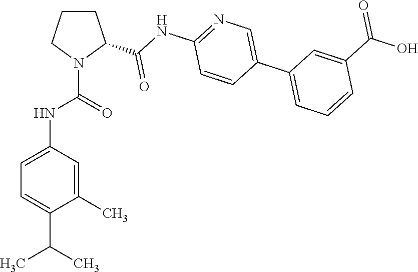 [SMILES: Cc1cc(NC(=O)N2CCC[C@@H]2C(=O)Nc2ccc(-c3cccc(C(=O)O)c3)cn2)ccc1C(C)C] | ¹H NMR (400 MHz, DMSO-d₆) δ 10.63 (s, 1H), 8.68 (dd, J = 2.3, 1.1 Hz, 1H), 8.21-8.14 (m, 3H), 8.13 (s, 1H), 7.98-7.93 (m, 2H), 7.61 (t, J = 7.7 Hz, 1H), 7.29-7.25 (m, 2H), 7.07 (d, J = 8.7 Hz, 1H), 4.65-4.58 (m, 1H), 3.67- 3.59 (m, 1H), 3.55-3.46 (m, 1H), 3.01 (septet, J = 6.8 Hz, 1H), 2.23 (s, 3H), 2.22- 2.10 (m, 1H), 2.07-1.89 (m, 3H), 1.14 (d, J = 6.8 Hz, 6H); 487.4 |
| 184 | 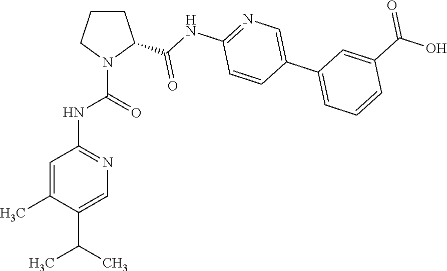 [SMILES: Cc1cc(NC(=O)N2CCC[C@@H]2C(=O)Nc2ccc(-c3cccc(C(=O)O)c3)cn2)ncc1C(C)C] | ¹H NMR (400 MHz, chloroform-d), characteristic peaks: δ 10.18 (s, 1H), 8.96 (s, 1H), 8.86 (s, 1H), 8.33 (d, J = 8.7 Hz, 1H), 8.10 (d, J = 7.7 Hz, 1H), 8.05-7.98 (m, 2H), 7.89 (s, 1H), 7.76 (d, J = 7.7 Hz, 1H), 7.55 (t, J = 7.8 Hz, 1H), 4.94-4.87 (m, 1H), 3.93- 3.84 (m, 1H), 3.78-3.68 (m, 1H), 3.12-3.02 (m, 1H), 2.40 (s, 3H), 2.30-2.23 (m, 3H), 2.05-1.95 (m, 1H), 1.25 (d, J = 6.9 Hz, 3H), 1.22 (d, J = 6.9 Hz, 3H); 488.4 |
| 185 | 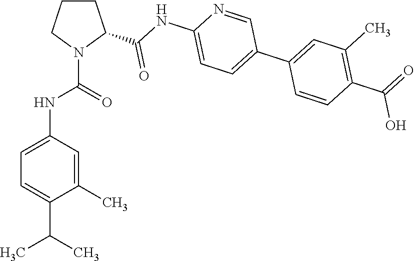 [SMILES: Cc1cc(-c2ccc(NC(=O)[C@H]3CCCN3C(=O)Nc3ccc(C(C)C)c(C)c3)nc2)ccc1C(=O)O] | ¹H NMR, characteristic peaks: δ 10.64 (s, 1H), 8.71 (s, 1H), 8.18-8.15 (m, 2H), 8.13 (s, 1H), 7.91 (d, J = 8.1 Hz, 1H), 7.68 (br s, 1H), 7.63 (dd, J = 8.2, 1.9 Hz, 1H), 7.28-7.23 (m, 2H), 7.07 (d, J = 8.2 Hz, 1H), 4.64-4.57 (m, 1H), 3.66-3.58 (m, 1H, assumed; partially obscured by water peak), 3.05-2.97 (m, 1H), 2.59 (s, 3H), 2.22 (s, 3H), 2.20-2.12 (m, 1H), 2.05-1.90 (m, 3H), 1.13 (d, J = 6.8 Hz, 6H); 501.5 |
| 186 | 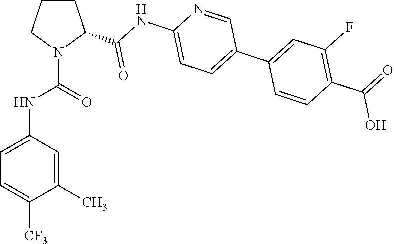 [SMILES: Cc1cc(NC(=O)N2CCC[C@@H]2C(=O)Nc2ccc(-c3ccc(C(=O)O)c(F)c3)cn2)ccc1C(F)(F)F] | ¹H NMR, characteristic peaks: δ 10.75 (s, 1H), 8.80-8.77 (m, 1H), 8.62 (s, 1H), 8.19 (AB quartet, downfield doublet is broadened, J_{AB} = 8.7 Hz, Δv_{AB} = 32.1 Hz, 2H), 7.94 (t, J = 8.0 Hz, 1H), 7.74 (br d, J = 12.2 Hz, 1H), 7.69 (br d, J = 8.2 Hz, 1H), 7.59 (br s, 1H), 7.52 (AB quartet, downfield doublet is broadened, J_{AB} = 8.9 Hz, Δv_{AB} = 15.9 Hz, 2H), 4.66-4.59 (m, 1H), 3.69-3.62 (m, 1H, assumed; partially obscured by water peak), 2.36 (s, 3H), 2.24- 2.17 (m, 1H), 2.05-1.89 (m, 3H); 531.4 |
| 187 | 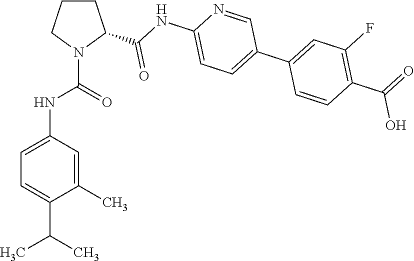 [SMILES: Cc1cc(NC(=O)N2CCC[C@@H]2C(=O)Nc2ccc(-c3ccc(C(=O)O)c(F)c3)cn2)ccc1C(C)C] | ¹H NMR, characteristic peaks: δ 10.69 (s, 1H), 8.79-8.75 (m, 1H), 8.19 (AB quartet, downfield doublet is broadened, J_{AB} = 8.6 Hz Δv_{AB} = 30.1 Hz, 2H), 8.14 (s, 1H), 7.94 (t, J = 8.0 Hz, 1H), 7.73 (br d, J = 12.3 Hz, 1H), 7.68 (br d, J = 8.2 Hz, 1H), 7.28-7.23 (m, 2H), 7.07 (d, J = 8.2 Hz, 1H), 4.64-4.57 (m, 1H), 3.66-3.57 (m, 1H, assumed; partially obscured by water peak), 3.05-2.97 (m, 1H), 2.22 (s, 3H), 2.20-2.13 (m, 1H), 2.04-1.90 (m, 3H), 1.13 (d, J = 6.8 Hz, 6H); 505.5 |
| 188 | 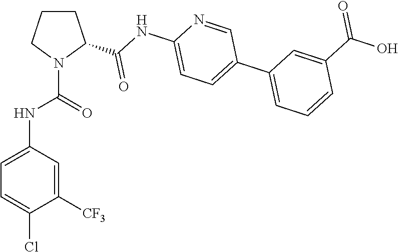 [SMILES: O=C(O)c1cccc(-c2ccc(NC(=O)[C@H]3CCCN3C(=O)Nc3ccc(Cl)c(C(F)(F)F)c3)nc2)c1] | ¹H NMR, characteristic peaks: δ 10.70 (s, 1H), 8.79 (s, 1H), 8.70-8.67 (m, 1H), 8.21-8.18 (m, 1H), 8.18-8.13 (m, 2H), 8.12 (br s, 1H), 7.99-7.93 (m, 2H), 7.83 (br d, J = 8.8 Hz, 1H), 7.61 (t, J = 7.7 Hz, 1H), 7.56 (d, J = 8.9 Hz, 1H), 4.67-4.59 (m, 1H), 3.68-3.60 (m, 1H, assumed; partially obscured by water peak), 2.26-2.17 (m, 1H), 2.07-1.90 (m, 3H); 533.4 (chlorine isotope pattern observed) |
| 189 | 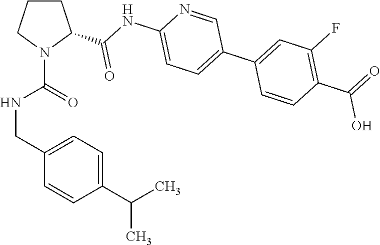 [SMILES: CC(C)c1ccc(CNC(=O)N2CCC[C@@H]2C(=O)Nc2ccc(-c3ccc(C(=O)O)c(F)c3)cn2)cc1] | ¹H NMR, characteristic peaks: δ 10.65 (s, 1H), 8.77 (d, J = 2.6 Hz, 1H), 8.23 (dd, component of ABX quartet, J = 8.7, 2.6 Hz, 1H), 8.17 (d, half of AB quartet, J = 8.7 Hz, 1H), 7.94 (t, J = 8.0 Hz, 1H), 7.74 (dd, J = 12.3, 1.7 Hz, 1H), 7.69 (dd, J = 8.2, 1.7 Hz, 1H), 7.16 (AB quartet, J_{AB} = 8.0 Hz, Δv_{AB} = 25.2 Hz, 4H), 6.86 (br t, J = 6.0 Hz, 1H), 4.54 (dd, J = 8.4, 2.9 Hz, 1H), 4.24 (dd, component of ABX system, J = 15.3, 6.1 Hz, 1H), 4.16 (dd, component of ABX system, J = 15.3, 5.8 Hz, 1H), 2.83 (septet, J = 6.9 Hz, 1H), 2.15-2.04 (m, 1H), 2.00-1.86 (m, 3H), 1.16 (d, J = 6.9 Hz, 6H); 505.6 |
| 190 | 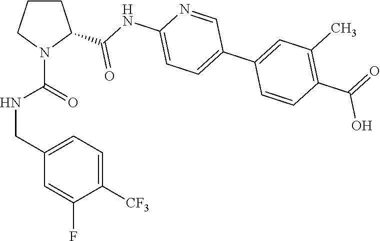 [SMILES: Cc1cc(-c2ccc(NC(=O)[C@H]3CCCN3C(=O)NCc3ccc(C(F)(F)F)c(F)c3)nc2)ccc1C(=O)O] | ¹H NMR, characteristic peaks: δ 10.60 (s, 1H), 8.72-8.68 (m, 1H), 8.16 (br s, 2H), 7.91 (d, J = 8.1 Hz, 1H), 7.70 (t, J = 8.0 Hz, 1H), 7.68 (br s, 1H), 7.63 (dd, J = 8.1, 2.0 Hz, 1H), 7.37 (d, J = 11.9 Hz, 1H), 7.31 (d, J = 8.0 Hz, 1H), 7.07 (br t, J = 6.1 Hz, 1H), 4.56-4.51 (m, 1H), 4.35 (dd, component of ABX system, J = 16.4, 5.9 Hz, 1H), 4.29 (dd, component of ABX system, J = 16.4, 6.0 Hz, 1H), 3.53- 3.47 (m, 1H), 2.60 (s, 3H), 2.18-2.08 (m, 1H), 2.00-1.89 (m, 3H); 545.5 |
| 191 | 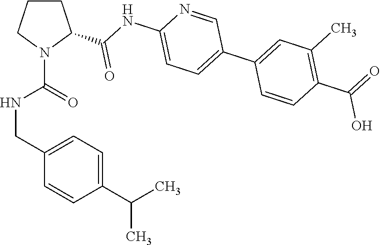 [SMILES: Cc1cc(-c2ccc(NC(=O)[C@H]3CCCN3C(=O)NCc3ccc(C(C)C)cc3)nc2)ccc1C(=O)O] | ¹H NMR, characteristic peaks: δ 10.60 (s, 1H), 8.72-8.70 (m, 1H), 8.17 (s, 2H), 7.92 (d, J = 8.1 Hz, 1H), 7.68 (s, 1H), 7.64 (br d, J = 8.2 Hz, 1H), 7.16 (AB quartet, J_{AB} = 7.9 Hz, Δv_{AB} = 26.2 Hz, 4H), 6.86 (br t, J = 6.0 Hz, 1H), 4.54 (dd, J = 8.4, 2.9 Hz, 1H), 4.24 (dd, component of ABX system, J = 15.3, 6.1 Hz, 1H), 4.16 (dd, component of ABX system, J = 15.3, 5.8 Hz, 1H), 3.50-3.43 (m, 1H, assumed; partially obscured by water peak), 2.83 (septet, J = 6.9 Hz, 1H), 2.60 (s, 3H), 2.14- 2.04 (m, 1H), 2.01-1.86 (m, 3H), 1.16 (d, J = 6.9 Hz, 6H); 501.6 |
| 192 | 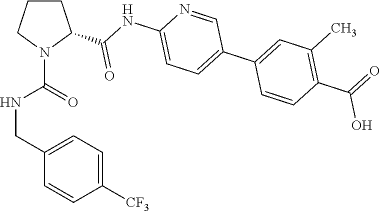 [SMILES: Cc1cc(-c2ccc(NC(=O)[C@H]3CCCN3C(=O)NCc3ccc(C(F)(F)F)cc3)nc2)ccc1C(=O)O] | ¹H NMR, characteristic peaks: δ 10.60 (s, 1H), 8.72-8.69 (m, 1H), 8.16 (s, 2H), 7.91 (d, J = 8.1 Hz, 1H), 7.68 (br s, 1H), 7.66-7.62 (m, 3H), 7.49 (d, J = 8.0 Hz, 2H), 7.04 (br t, J = 6.0 Hz, 1H), 4.56-4.51 (m, 1H), 4.36 (dd, component of ABX system, J = 16.0, 5.9 Hz, 1H), 4.28 (dd, component of ABX system, J = 16.1, 5.8 Hz, 1H), 3.53-3.46 (m, 1H, assumed; partially obscured by water peak), 2.60 (s, 3H), 2.16-2.07 (m, 1H), 2.00-1.88 (m, 3H); 527.5 |
| 193 | 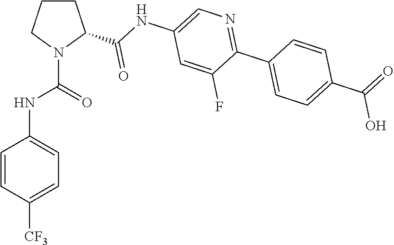 [SMILES: O=C(O)c1ccc(-c2ncc(NC(=O)[C@H]3CCCN3C(=O)Nc3ccc(C(F)(F)F)cc3)cc2F)cc1] | ¹H NMR, characteristic peaks: δ 10.69 (s, 1H), 8.74 (s, 1H), 8.71 (s, 1H), 8.20 (dd, J = 13.8, 2.0 Hz, 1H), 8.04 (AB quartet, upfield doublet is broadened, J_{AB} = 8.3 Hz, Δv_{AB} = 18.6 Hz, 4H), 7.75 (d, J = 8.5 Hz, 2H), 7.58 (d, J = 8.6 Hz, 2H), 4.53-4.48 (m, 1H), 3.73-3.65 (m, 1H, assumed; partially obscured by water peak), 2.30-2.20 (m, 1H); 517.3 |
| 194 | 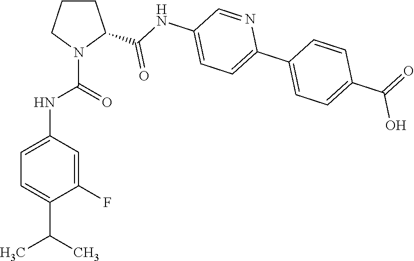 [SMILES: CC(C)c1ccc(NC(=O)N2CCC[C@@H]2C(=O)Nc2ccc(-c3ccc(C(=O)O)cc3)nc2)cc1F] | 10.40 (s, 1H), 8.88 (d, J = 2.5 Hz, 1H), 8.43 (s, 1H), 8.21-8.18 (m, 1H), 8.16 (d, J = 8.3 Hz, 2H), 8.05-8.00 (m, 3H), 7.40 (br d, J = 13.6 Hz, 1H), 7.26-7.21 (m, 1H), 7.16 (t, J = 8.7 Hz, 1H), 4.48 (dd, J = 8.4, 3.8 Hz, 1H), 3.68- 3.61 (m, 1H), 3.56-3.49 (m, 1H, assumed; partially obscured by water peak), 3.06 (septet, J = 6.9 Hz, 1H), 2.26-2.18 (m, 1H), 2.08-1.91 (m, 3H), 1.17 (d, J = 6.9 Hz, 6H); 491.5 |
| 195 | 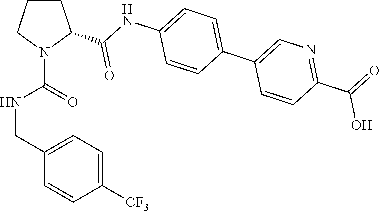 [SMILES: O=C(O)c1ccc(-c2ccc(NC(=O)[C@H]3CCCN3C(=O)NCc3ccc(C(F)(F)F)cc3)cc2)cn1] | ¹H NMR, characteristic peaks: δ 10.14 (s, 1H), 9.02-8.97 (m, 1H), 8.23 (dd, J = 8.2, 2.3 Hz, 1H), 8.09 (br d, J = 8.2 Hz, 1H), 7.80-7.76 (m, 2H), 7.76-7.72 (m, 2H), 7.66-7.62 (m, 2H), 7.49 (d, J = 7.8 Hz, 2H), 7.02 (br t, J = 6.0 Hz, 1H), 4.42-4.37 (m, 1H), 4.35 (dd, component of ABX system, J = 15.8, 5.4 Hz, 1H), 4.28 (dd, component of ABX system, J = 16.0, 5.9 Hz, 1H), 2.19-2.00 (m, 1H), 2.04- 1.88 (m, 3H); 513.4 |
| 196 | 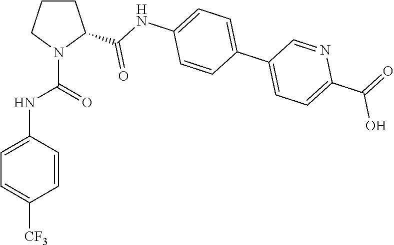 [SMILES: O=C(O)c1ccc(-c2ccc(NC(=O)[C@H]3CCCN3C(=O)Nc3ccc(C(F)(F)F)cc3)cc2)cn1] | ¹H NMR, characteristic peaks: δ 10.23 (s, 1H), 9.02-8.99 (m, 1H), 8.71 (s, 1H), 8.24 (dd, J = 8.2, 2.4 Hz, 1H), 8.08 (d, J = 8.2 Hz, 1H), 7.79 (br AB quartet, J_{AB} = 8.7 Hz, Δv_{AB} = 10.7 Hz, 4H), 7.75 (br d, J = 8 Hz, 2H), 7.58 (d, J = 8.5 Hz, 2H), 4.53-4.47 (m, 1H), 3.72-3.65 (m, 1H, assumed; partially obscured by water peak), 2.27-2.18 (m, 1H), 2.09-1.90 (m, 3H); 499.3 |
| 197 | 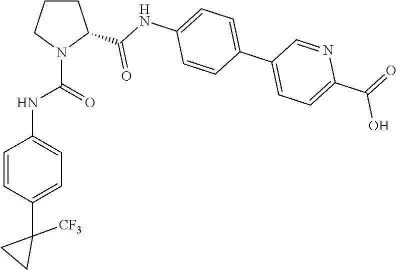 [SMILES: O=C(O)c1ccc(-c2ccc(NC(=O)[C@H]3CCCN3C(=O)Nc3ccc(C4(C(F)(F)F)CC4)cc3)cc2)cn1] | ¹H NMR, characteristic peaks: δ 10.21 (s, 1H), 9.02-8.98 (m, 1H), 8.39 (s, 1H), 8.23 (dd, J = 8.2, 2.4 Hz, 1H), 8.08 (d, J = 8.1 Hz, 1H), 7.81- 7.75 (m, 4H), 7.53-7.48 (m, 2H), 7.30 (d, J = 8.3 Hz, 2H), 4.51-4.45 (m, 1H), 3.69- 3.62 (m, 1H, assumed; partially obscured by water peak), 2.25-2.17 (m, 1H), 2.08-1.89 (m, 3H), 1.29-1.24 (m, 2H), 1.07-1.01 (m, 2H); 539.3 |
| 198 | 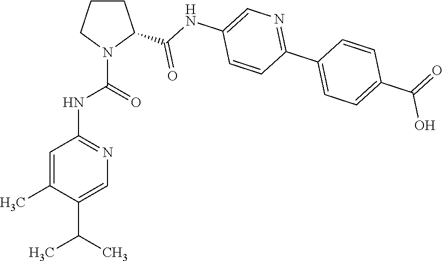 [SMILES: Cc1cc(NC(=O)N2CCC[C@@H]2C(=O)Nc2ccc(-c3ccc(C(=O)O)cc3)nc2)ncc1C(C)C] | ¹H NMR (400 MHz, DMSO-d₆) δ 10.38 (s, 1H), 8.90-8.86 (m, 1H), 8.59 (s, 1H), 8.24-8.12 (m, 3H), 8.08-7.98 (m, 4H), 7.63 (s, 1H), 4.56-4.46 (m, 1H), 3.74-3.63 (m, 1H), 3.62- 3.53 (m, 1H, assumed; partially obscured by water peak), 3.09-2.97 (m, 1H), 2.24 (s, 3H), 2.08-1.88 (m, 4H), 1.20 (d, J = 6.9 Hz, 6H); 488.4 |
| 199 | 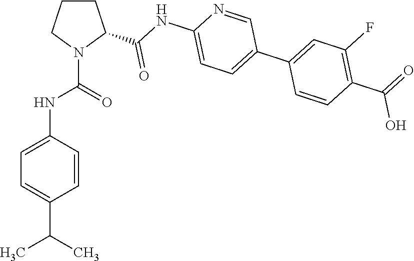 [SMILES: CC(C)c1ccc(NC(=O)N2CCC[C@@H]2C(=O)Nc2ccc(-c3ccc(C(=O)O)c(F)c3)cn2)cc1] | ¹H NMR, characteristic peaks: δ 10.72-10.66 (m, 1H), 8.79-8.74 (m, 1H), 8.25-8.19 (m, 2H), 8.16 (d, half of AB quartet, J = 8.7 Hz, 1H), 7.94 (t, J = 8.1 Hz, 1H), 7.75-7.70 (m, 1H), 7.68 (br d, J = 8.0 Hz, 1H), 7.41-7.35 (m, 2H), 7.09 (d, J = 8.3 Hz, 2H), 4.66-4.57 (m, 1H), 2.79 (septet, J = 7.0 Hz, 1H), 2.22- 2.12 (m, 1H), 2.05-1.90 (m, 3H), 1.18-1.13 (m, 6H); 491.3 |
| 200 | 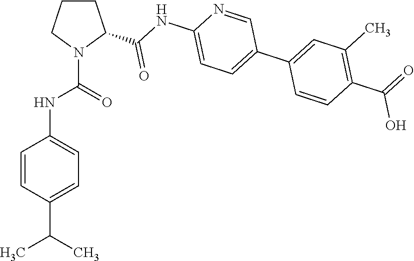 [SMILES: Cc1cc(-c2ccc(NC(=O)[C@H]3CCCN3C(=O)Nc3ccc(C(C)C)cc3)nc2)ccc1C(=O)O] | ¹H NMR, characteristic peaks: δ 10.66-10.62 (m, 1H), 8.73-8.68 (m, 1H), 8.22 (s, 1H), 8.16 (br s, 2H), 7.91 (d, J = 8.1 Hz, 1H), 7.68 (br s, 1H), 7.63 (br d, J = 8.2 Hz, 1H), 7.40- 7.36 (m, 2H), 7.09 (d, J = 8.5 Hz, 2H), 4.65- 4.58 (m, 1H), 3.67-3.59 (m, 1H, assumed; partially obscured by water peak), 2.80 (septet, J = 6.8 Hz, 1H), 2.59 (s, 3H), 2.22- 2.12 (m, 1H), 2.05-1.90 (m, 3H), 1.16 (d, J = 6.9 Hz, 6H); 487.3 |
| 201 | 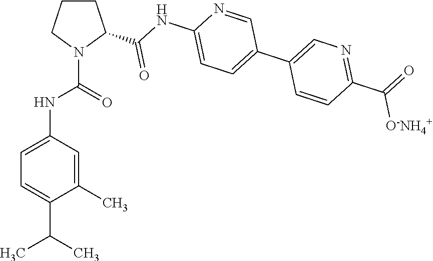 | ¹H NMR, characteristic peaks: δ 10.70 (s, 1H), 9.08-9.04 (m, 1H), 8.81-8.78 (m, 1H), 8.31 (dd, J = 8.2, 2.3 Hz, 1H), 8.26 (dd, component of ABX system, J = 8.7, 2.5 Hz, 1H), 8.20 (d, half of AB quartet, J = 8.7 Hz, 1H), 8.14 (s, 1H), 8.11 (d, J = 8.2 Hz, 1H), 7.28-7.22 (m, 2H), 7.06 (d, J = 8.2 Hz, 1H), 4.64-4.58 (m, 1H), 3.01 (septet, J = 6.8 Hz, 1H), 2.22 (s, 3H), 2.21-2.12 (m, 1H), 2.05-1.90 (m, 3H), 1.12 (d, J = 6.8 Hz, 6H); 488.6 |
| 202 | 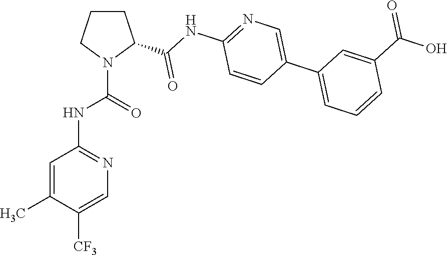 [SMILES: Cc1cc(NC(=O)N2CCC[C@@H]2C(=O)Nc2ccc(-c3cccc(C(=O)O)c3)cn2)ncc1C(F)(F)F] | ¹H NMR (400 MHz, DMSO-d₆) δ 10.73 (s, 1H), 9.34 (s, 1H), 8.70 (br t, J = 1.7 Hz, 1H), 8.47 (s, 1H), 8.21 (br t, J = 1.8 Hz, 1H), 8.19-8.13 (m, 2H), 7.99-7.91 (m, 3H), 7.61 (t, J = 7.8 Hz, 1H), 4.73-4.61 (m, 1H), 3.76-3.66 (m, 1H), 3.66-3.56 (m, 1H), 2.39 (s, 3H), 2.29- 2.16 (m, 1H), 2.06-1.87 (m, 3H); 514.3 |
| 203 | 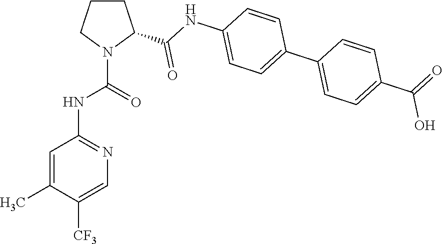 [SMILES: Cc1cc(NC(=O)N2CCC[C@@H]2C(=O)Nc2ccc(-c3ccc(C(=O)O)cc3)cc2)ncc1C(F)(F)F] | ¹H NMR (400 MHz, DMSO-d₆) δ 10.19 (s, 1H), 9.33 (br s, 1H), 8.47 (s, 1H), 7.98 (d, J = 8.2 Hz, 2H), 7.95 (s, 1H), 7.76 (d, J = 8.2 Hz, 2H), 7.75-7.68 (m, 4H), 4.58-4.48 (m, 1H), 3.77- 3.66 (m, 1H), 3.66-3.56 (m, 1H), 2.39 (s, 3H), 2.30-2.17 (m, 1H), 2.07-1.87 (m, 3H); 513.3 |
| 204 | 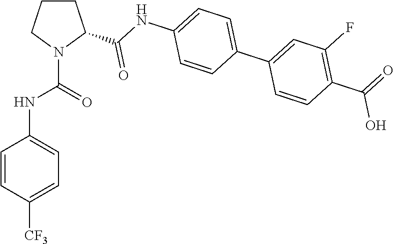 [SMILES: O=C(O)c1ccc(-c2ccc(NC(=O)[C@H]3CCCN3C(=O)Nc3ccc(C(F)(F)F)cc3)cc2)cc1F] | ¹H NMR, characteristic peaks: δ 10.20 (s, 1H), 8.70 (s, 1H), 7.91 (t, J = 8.0 Hz, 1H), 7.78- 7.71 (m, 6H), 7.64-7.59 (m, 2H), 7.57 (d, J = 8.6 Hz, 2H), 4.52-4.46 (m, 1H), 3.72-3.64 (m, 1H, assumed; partially obscured by water peak), 2.26-2.18 (m, 1H), 2.08-1.89 (m, 3H); 516.3 |
| 205 | 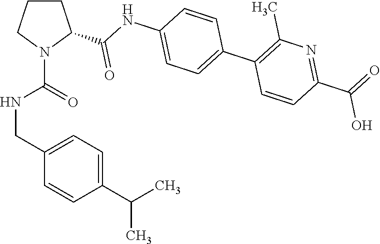 [SMILES: Cc1nc(C(=O)O)ccc1-c1ccc(NC(=O)[C@H]2CCCN2C(=O)NCc2ccc(C(C)C)cc2)cc1] | ¹H NMR, characteristic peaks: δ 10.10 (s, 1H), 7.93 (d, J = 7.9 Hz, 1H), 7.76 (d, J = 7.9 Hz, 1H), 7.73-7.69 (m, 2H), 7.39 (d, J = 8.2 Hz, 2H), 7.16 (AB quartet, J_{AB} = 7.7 Hz, Δv_{AB} = 27.7 Hz, 4H), 6.83 (br t, J = 6.0 Hz, 1H), 4.39 (dd, J = 8.3, 2.9 Hz, 1H), 4.23 (dd, component of ABX system, J = 15.3, 5.8 Hz, 1H), 4.16 (dd, component of ABX system, J = 15.3, 5.7 Hz, 1H), 2.83 (septet, J = 6.9 Hz, 1H), 2.17- 2.07 (m, 1H), 2.03-1.86 (m, 3H), 1.19-1.13 (m, 6H); 501.6 |
| 206 | 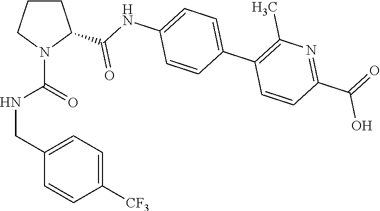 [SMILES: Cc1nc(C(=O)O)ccc1-c1ccc(NC(=O)[C@H]2CCCN2C(=O)NCc2ccc(C(F)(F)F)cc2)cc1] | ¹H NMR, characteristic peaks: δ 10.09 (s, 1H), 7.93 (d, J = 7.8 Hz, 1H), 7.76 (d, J = 7.9 Hz, 1H), 7.71 (d, J = 8.5 Hz, 2H), 7.64 (d, J = 8.0 Hz, 2H), 7.49 (d, J = 7.9 Hz, 2H), 7.38 (d, J = 8.6 Hz, 2H), 7.01 (t, J = 6.0 Hz, 1H), 4.39 (dd, J = 8.5, 3.1 Hz, 1H), 4.35 (dd, component of ABX system, J = 16.1, 6.0 Hz, 1H), 4.28 (dd, component of ABX system J = 16.0, 5.9 Hz, 1H), 2.19-2.09 (m, 1H), 2.04-1.88 (m, 3H); 527.5 |
| 207 | 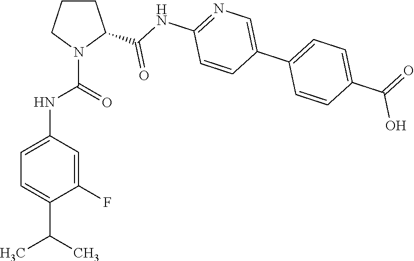 [SMILES: CC(C)c1ccc(NC(=O)N2CCC[C@@H]2C(=O)Nc2ccc(-c3ccc(C(=O)O)cc3)cn2)cc1F] | ¹H NMR, characteristic peaks: δ 10.68 (s, 1H), 8.74-8.71 (m, 1H), 8.42 (s, 1H), 8.17 (br s, 2H), 8.02 (d, J = 8.4 Hz, 2H), 7.85 (d, J = 8.2 Hz, 2H), 7.39 (br d, J = 13 Hz, 1H), 7.23 (br d, half of AB quartet, J = 8.7 Hz, 1H), 7.16 (dd, component of ABX system, J = 8.8, 8.6 Hz, 1H), 4.65-4.58 (m, 1H), 3.10-3.01 (m, 1H), 2.23-2.14 (m, 1H), 2.05-1.89 (m, 3H), 1.17 (d, J = 6.9 Hz, 6H); 491.3 |
| 208 | 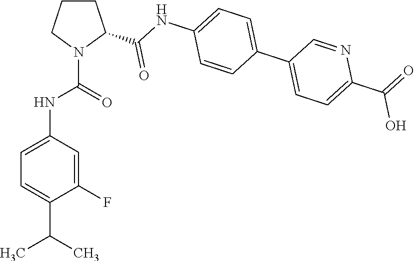 [SMILES: CC(C)c1ccc(NC(=O)N2CCC[C@@H]2C(=O)Nc2ccc(-c3ccc(C(=O)O)nc3)cc2)cc1F] | 10.20 (s, 1H), 9.01 (dd, J = 2.3, 0.8 Hz, 1H), 8.41 (s, 1H), 8.24 (dd, J = 8.2, 2.4 Hz, 1H), 8.09 (br d, J = 8.2 Hz, 1H), 7.79 (AB quartet, J_{AB} = 9.0 Hz, Δv_{AB} = 11.2 Hz, 4H), 7.40 (dd, J = 13.6, 2.1 Hz, 1H), 7.24 (dd, half of AB quartet, J = 8.5, 2.1 Hz, 1H), 7.16 (dd, component of ABX system, J = 8.6, 8.6 Hz, 1H), 4.47 (dd, J = 8.4, 3.9 Hz, 1H), 3.68-3.61 (m, 1H), 3.56-3.48 (m, 1H, assumed; partially obscured by water peak), 3.06 (septet, J = 6.9 Hz, 1H), 2.25-2.16 (m, 1H), 2.08-1.89 (m, 3H), 1.17 (d, J = 6.9 Hz, 6H); 491.4 |
| 209 | 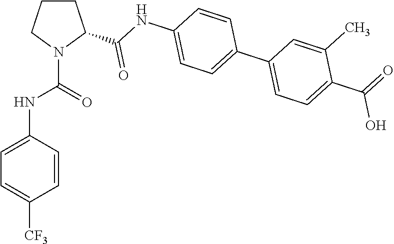 [SMILES: Cc1cc(-c2ccc(NC(=O)[C@H]3CCCN3C(=O)Nc3ccc(C(F)(F)F)cc3)cc2)ccc1C(=O)O] | 10.15 (s, 1H), 8.70 (s, 1H), 7.89 (d, J = 8.1 Hz, 1H), 7.75 (d, J = 8.6 Hz, 2H), 7.71 (AB quartet, J_{AB} = 8.9 Hz, Δv_{AB} = 15.1 Hz, 4H), 7.61-7.55 (m, 4H), 4.49 (dd, J = 8.3, 3.9 Hz, 1H), 3.72-3.65 (m, 1H), 3.60-3.53 (m, 1H), 2.59 (s, 3H), 2.27-2.18 (m, 1H), 2.09-2.00 (m, 1H), 2.00-1.90 (m, 2H); 512.4 |
| 210 | 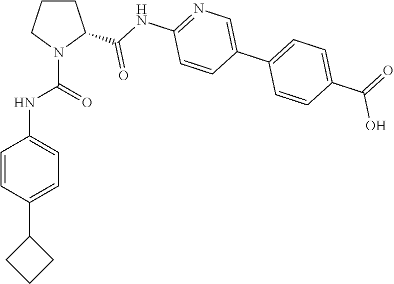 [SMILES: O=C(O)c1ccc(-c2ccc(NC(=O)[C@H]3CCCN3C(=O)Nc3ccc(C4CCC4)cc3)nc2)cc1] | ¹H NMR, characteristic peaks: δ 10.67-10.63 (m, 1H), 8.75-8.70 (m, 1H), 8.24 (s, 1H), 8.17 (br s, 2H), 8.02 (d, J = 8.4 Hz, 2H), 7.85 (br d, J = 8.2 Hz, 2H), 7.42-7.37 (m, 2H), 7.08 (d, J = 8.4 Hz, 2H), 4.65-4.57 (m, 1H), 2.27-2.20 (m, 2H), 2.20-2.13 (m, 1H), 2.07- 1.89 (m, 6H), 1.81-1.73 (m, 1H); 485.3 |
| 211 | 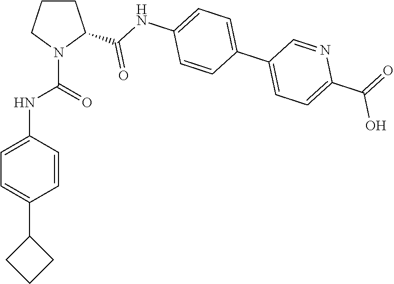 [SMILES: O=C(O)c1ccc(-c2ccc(NC(=O)[C@H]3CCCN3C(=O)Nc3ccc(C4CCC4)cc3)cc2)cn1] | ¹H NMR, characteristic peaks: δ 10.20 (s, 1H), 9.01 (d, J = 2.4 Hz, 1H), 8.24 (dd, J = 8.2, 2.4 Hz, 1H), 8.22 (s, 1H), 8.08 (d, J = 8.2 Hz, 1H), 7.79 (AB quartet, J_{AB} = 9.0 Hz, Δv_{AB} = 9.8 Hz, 4H), 7.41 (d, J = 8.5 Hz, 2H), 7.08 (d, J = 8.6 Hz, 2H), 4.47 (dd, J = 8.2, 3.8 Hz, 1H), 3.67- 3.61 (m, 1H), 3.55-3.48 (m, 1H, assumed; partially obscured by water peak), 2.27-2.15 (m, 3H), 2.07-1.88 (m, 6H), 1.81-1.73 (m, 1H); 485.4 |
| 212 | 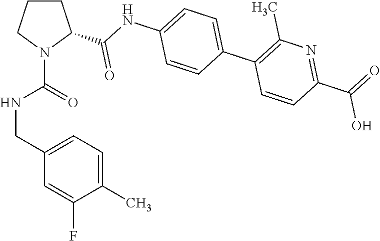 [SMILES: Cc1ccc(CNC(=O)N2CCC[C@@H]2C(=O)Nc2ccc(-c3ccc(C(=O)O)nc3C)cc2)cc1F] | ¹H NMR, characteristic peaks: δ 10.09 (s, 1H), 7.92 (d, J = 7.8 Hz, 1H), 7.76 (d, J = 7.9 Hz, 1H), 7.71 (d, J = 8.6 Hz, 2H), 7.38 (d, J = 8.6 Hz, 2H), 7.18 (t, J = 7.9 Hz, 1H), 7.02 (br d, J = 11.0 Hz, 1H), 7.00 (br d, J = 7.7 Hz, 1H), 6.90 (t, J = 6.1 Hz, 1H), 4.39 (dd, J = 8.5, 3.1 Hz, 1H), 4.23 (dd, component of ABX system, J = 15.7, 6.0 Hz, 1H), 4.18 (dd, component of ABX system, J = 15.7, 6.0 Hz, 1H), 2.18 (s, 3H), 2.17-2.09 (m, 1H), 2.01-1.88 (m, 3H); 491.5 |
| 213 | 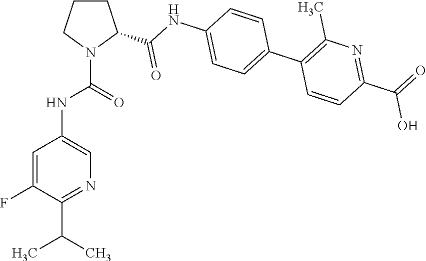 [SMILES: Cc1nc(C(=O)O)ccc1-c1ccc(NC(=O)[C@H]2CCCN2C(=O)Nc2cnc(C(C)C)c(F)c2)cc1] | ¹H NMR, characteristic peaks: δ 10.19 (s, 1H), 8.70 (s, 1H), 8.46 (s, 1H), 7.93 (d, J = 7.8 Hz, 1H), 7.86-7.81 (m, 1H), 7.77 (d, J = 7.9 Hz, 1H), 7.73 (d, J = 8.6 Hz, 2H), 7.39 (d, J = 8.6 Hz, 2H), 4.49 (dd, J = 8.4, 3.8 Hz, 1H), 3.69- 3.62 (m, 1H, assumed; partially obscured by water peak), 3.25-3.19 (m, 1H, assumed; partially obscured by water peak), 2.26-2.18 (m, 1H), 2.09-1.91 (m, 3H), 1.19 (d, J = 6.8 Hz, 6H); 506.6 |
| 214 | 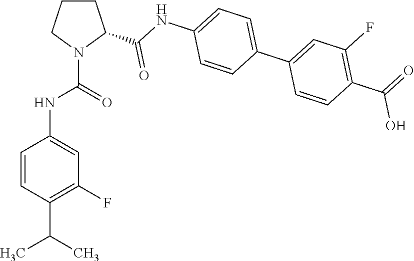 [SMILES: CC(C)c1ccc(NC(=O)N2CCC[C@@H]2C(=O)Nc2ccc(-c3ccc(C(=O)O)c(F)c3)cc2)cc1F] | ¹H NMR, characteristic peaks: δ 10.19 (s, 1H), 8.41 (s, 1H), 7.91 (t, J = 8.1 Hz, 1H), 7.74 (AB quartet, J_{AB} = 9.0 Hz, Δv_{AB} = 13.5 Hz, 4H), 7.62 (s, 1H), 7.61-7.59 (m, 1H), 7.38 (dd, J = 13.6, 2.1 Hz, 1H), 7.22 (dd, half of AB quartet, J = 8.5, 2.1 Hz, 1H), 7.15 (dd, component of ABX system, J = 8.7, 8.7 Hz, 1H), 4.46 (dd, J = 8.4, 4.0 Hz, 1H), 3.05 (septet, J = 7.0 Hz, 1H), 2.24-2.15 (m, 1H), 2.06-1.87 (m, 3H), 1.16 (d, J = 6.9 Hz, 6H); 508.4 |
| 215 | 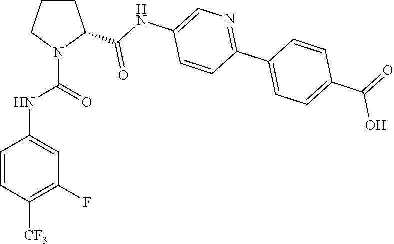 [SMILES: O=C(O)c1ccc(-c2ccc(NC(=O)[C@H]3CCCN3C(=O)Nc3ccc(C(F)(F)F)c(F)c3)cn2)cc1] | ¹H NMR (400 MHz, DMSO-d₆) δ 12.98 (br s, 1H), 10.42 (s, 1H), 8.94 (s, 1H), 8.88 (d, J = 2.6 Hz, 1H), 8.20 (dd, J = 8.8, 2.6 Hz, 1H), 8.17 (d, J = 8.7 Hz, 2H), 8.04 (d, J = 8.4 Hz, 1H), 8.02 (d, J = 8.6 Hz, 2H), 7.76 (br d, J = 14.3 Hz, 1H), 7.62 (dd, component of ABX system, J = 8.7, 8.7 Hz, 1H), 7.52 (br d, half of AB quartet, J = 8.9 Hz, 1H), 4.52 (dd, J = 8.6, 3.6 Hz, 1H), 3.73-3.63 (m, 1H), 3.63-3.53 (m, 1H), 2.31-2.19 (m, 1H), 2.11-1.92 (m, 3H); 517.4 |
| 216 | 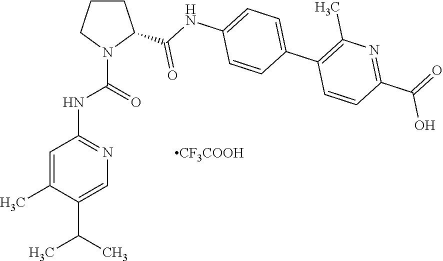 [SMILES: Cc1cc(NC(=O)N2CCC[C@@H]2C(=O)Nc2ccc(-c3ccc(C(=O)O)nc3C)cc2)ncc1C(C)C ; "·CF3COOH"] | ¹H NMR, characteristic peaks: δ 10.29 (s, 1H), 8.03 (s, 1H), 7.93 (d, J = 7.8 Hz, 1H), 7.76 (d, J = 7.9 Hz, 1H), 7.73 (d, J = 8.6 Hz, 2H), 7.55 (s, 1H), 7.40 (d, J = 8.6 Hz, 2H), 4.59-4.50 (m, 1H), 3.73-3.66 (m, 1H, assumed; partially obscured by water peak), 3.09 (septet, J = 6.9 Hz, 1H), 2.42 (s, 3H), 2.32- 2.23 (m, 1H), 2.11-1.94 (m, 3H), 1.20 (d, J = 6.8 Hz, 6H); 502.4 |
| 217 | 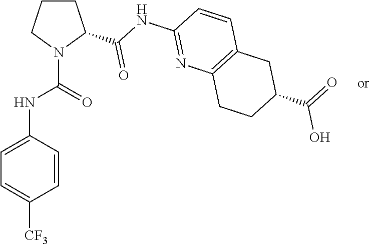 [SMILES: O=C(O)[C@@H]1CCc2nc(NC(=O)[C@H]3CCCN3C(=O)Nc3ccc(C(F)(F)F)cc3)ccc2C1] | 10.36 (s, 1H), 8.68 (s, 1H), 7.79 (d, J = 8.4 Hz, 1H), 7.73 (d, J = 8.6 Hz, 2H), 7.57 (d, J = 8.6 Hz, 2H), 7.47 (d, J = 8.5 Hz, 1H), 4.58 (dd, J = 8.5, 4.0 Hz, 1H), 3.68-3.62 (m, 1H), 3.57- 3.51 (m, 1H, assumed; partially obscured by water peak), 2.87 (dd, component of ABX system, J = 16.7, 5.4 Hz, 1H), 2.84-2.75 (m, 3H), 2.66-2.59 (m, 1H), 2.21-2.08 (m, 2H), 2.02-1.85 (m, 3H), 1.85-1.77 (m, 1H); 477.3 |
| | 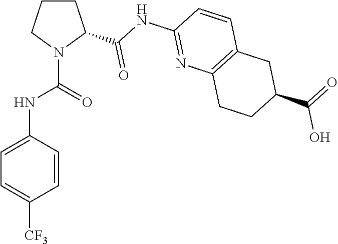 [SMILES: O=C(O)[C@H]1CCc2nc(NC(=O)[C@H]3CCCN3C(=O)Nc3ccc(C(F)(F)F)cc3)ccc2C1] | |
| | DIAST-1 (see footnote 43 in Table 2) | |
| 218 | 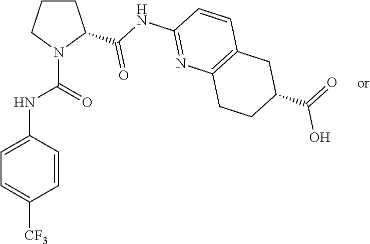 [SMILES: O=C(O)[C@@H]1CCc2nc(NC(=O)[C@H]3CCCN3C(=O)Nc3ccc(C(F)(F)F)cc3)ccc2C1] | ¹H NMR (600 MHz, DMSO_ASAP) δ 10.36 (s, 1H), 8.68 (s, 1H), 7.79 (d, J = 8.4 Hz, 1H), 7.73 (d, J = 8.5 Hz, 2H), 7.57 (d, J = 8.7 Hz, 2H), 7.46 (d, J = 8.4 Hz, 1H), 4.62-4.55 (m, 1H), 3.68-3.61 (m, 1H, assumed; partially obscured by water peak), 3.57-3.51 (m, 1H, assumed; partially obscured by water peak), 2.86 (dd, component of ABX system, J = 16.6, 5.5 Hz, 1H), 2.83-2.75 (m, 3H), 2.64-2.58 (m, 1H), 2.22-2.07 (m, 2H), 2.02-1.85 (m, 3H), 1.85-1.76 (m, 1H); 477.3 |
| | 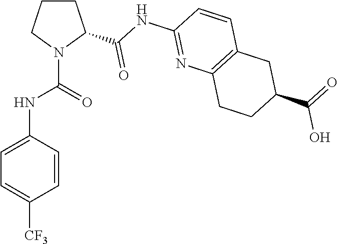 [SMILES: O=C(O)[C@H]1CCc2nc(NC(=O)[C@H]3CCCN3C(=O)Nc3ccc(C(F)(F)F)cc3)ccc2C1] | |
| | DIAST-2 (see footnote 4 in Table 2) | |
| 219 | 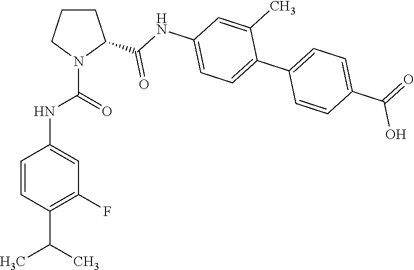 [SMILES: Cc1cc(NC(=O)[C@H]2CCCN2C(=O)Nc2ccc(C(C)C)c(F)c2)ccc1-c1ccc(C(=O)O)cc1] | ¹H NMR, characteristic peaks: δ 10.03 (s, 1H), 8.40 (s, 1H), 7.98 (d, J = 8.3 Hz, 2H), 7.57 (d, J = 2.2 Hz, 1H), 7.51 (dd, J = 8.4, 2.2 Hz, 1H), 7.44 (d, J = 8.4 Hz, 2H), 7.40 (dd, J = 13.6, 2.2 Hz, 1H), 7.23 (dd, J = 8.5, 2.1 Hz, 1H), 7.17 (d, J = 8.3 Hz, 1H), 7.15 (t, J = 8.6 Hz, 1H), 4.46 (dd, J = 8.3, 3.9 Hz, 1H), 3.66-3.60 (m, 1H, assumed; partially obscured by water peak), 3.06 (septet, J = 6.9 Hz, 1H), 2.21 (s, 3H), 2.21-2.15 (m, 1H), 2.07-1.87 (m, 3H), 1.17 (d, J = 6.9 Hz, 6H); 504.5 |
| 220 | 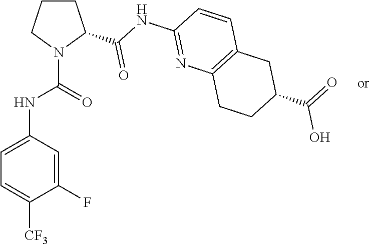 [SMILES: O=C(O)[C@@H]1CCc2nc(NC(=O)[C@H]3CCCN3C(=O)Nc3ccc(C(F)(F)F)c(F)c3)ccc2C1] | 10.39 (s, 1H), 8.92 (s, 1H), 7.78 (d, J = 8.3 Hz, 1H), 7.73 (d, J = 14.3 Hz, 1H), 7.60 (t, J = 8.7 Hz, 1H), 7.51-7.44 (m, 2H), 4.62-4.54 (m, 1H), 3.67-3.60 (m, 1H, assumed; partially obscured by water peak), 3.57-3.51 (m, 1H, assumed; partially obscured by water peak), 2.91-2.74 (m, 4H), 2.65-2.58 (m, 1H), 2.24- 2.07 (m, 2H), 2.03-1.76 (m, 4H); 495.3 |
| | 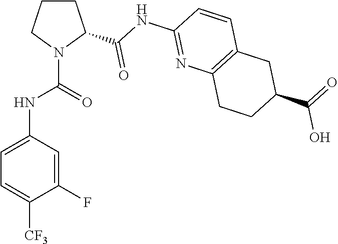 [SMILES: O=C(O)[C@H]1CCc2nc(NC(=O)[C@H]3CCCN3C(=O)Nc3ccc(C(F)(F)F)c(F)c3)ccc2C1] | |
| | DIAST-1 (see footnote 46 in Table 2) | |
| 221 | 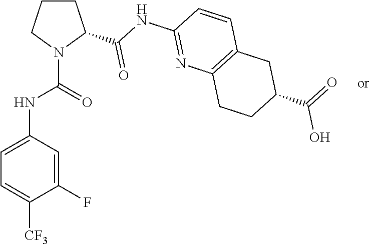 [SMILES: O=C(O)[C@@H]1CCc2nc(NC(=O)[C@H]3CCCN3C(=O)Nc3ccc(C(F)(F)F)c(F)c3)ccc2C1] | 10.40 (s, 1H), 8.92 (s, 1H), 7.78 (d, J = 8.4 Hz, 1H), 7.73 (d, J = 14.3, Hz, 1H), 7.60 (t, J = 8.7 Hz, 1H), 7.51-7.44 (m, 2H), 4.62-4.55 (m, 1H), 3.67-3.60 (m, 1H, assumed; partially obscured by water peak), 3.58-3.50 (m, 1H, assumed; partially obscured by water peak), 2.91-2.74 (m, 4H), 2.66-2.59 (m, 1H), 2.23- 2.07 (m, 2H), 2.02-1.76 (m, 4H); 495.3 |
| | 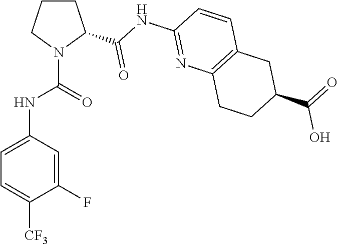 [SMILES: O=C(O)[C@H]1CCc2nc(NC(=O)[C@H]3CCCN3C(=O)Nc3ccc(C(F)(F)F)c(F)c3)ccc2C1] | |
| | DIAST-2 (see footnote 46 in Table 2) | |
| 222 | 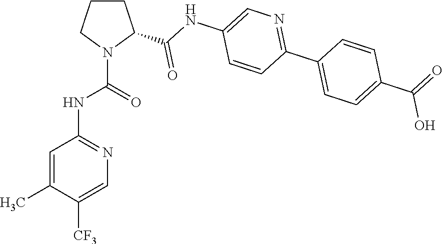 [SMILES: Cc1cc(NC(=O)N2CCC[C@@H]2C(=O)Nc2ccc(-c3ccc(C(=O)O)cc3)nc2)ncc1C(F)(F)F] | ¹H NMR (400 MHz, DMSO-d₆) δ 10.44 (s, 1H), 9.37 (s, 1H), 8.88 (d, J = 2.5 Hz, 1H), 8.47 (s, 1H), 8.20 (dd, J = 8.8, 2.6 Hz, 1H), 8.16 (d, J = 8.5 Hz, 2H), 8.06-7.99 (m, 3H), 7.94 (s, 1H), 4.60-4.49 (m, 1H), 3.78-3.67 (m, 1H), 3.67- 3.57 (m, 1H), 2.39 (s, 3H), 2.30-2.19 (m, 1H), 2.08-1.89 (m, 3H); 514.3 |
| 223 | 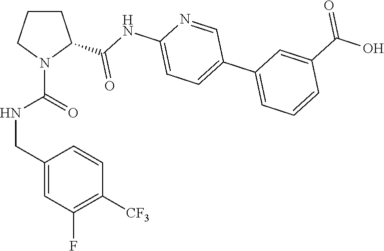 [SMILES: O=C(O)c1cccc(-c2ccc(NC(=O)[C@H]3CCCN3C(=O)NCc3ccc(C(F)(F)F)c(F)c3)nc2)c1] | ¹H NMR, characteristic peaks: δ 10.59 (s, 1H), 8.69-8.64 (m, 1H), 8.20 (br s, 1H), 8.17 (d, half of AB quartet, J = 8.8 Hz, 1H), 8.14 (dd, component of ABX system, J = 8.7, 2.4 Hz, 1H), 7.98-7.93 (m, 2H), 7.70 (t, J = 7.8 Hz, 1H), 7.61 (t, J = 7.8 Hz, 1H), 7.37 (d, J = 12.0 Hz, 1H), 7.31 (d, J = 8.0 Hz, 1H), 7.07 (br t, J = 6.0 Hz, 1H), 4.56-4.51 (m, 1H), 4.35 (dd, component of ABX system, J = 16.4, 5.8 Hz, 1H), 4.29 (dd, component of ABX system, J = 16.3, 6.0 Hz, 1H), 2.18-2.08 (m, 1H), 2.00- 1.88 (m, 3H); 531.3 |
| 224 | 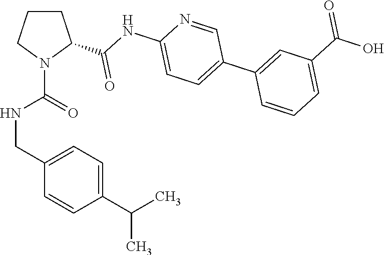 [SMILES: CC(C)c1ccc(CNC(=O)N2CCC[C@@H]2C(=O)Nc2ccc(-c3cccc(C(=O)O)c3)cn2)cc1] | ¹H NMR, characteristic peaks: δ 10.58 (s, 1H), 8.69-8.65 (m, 1H), 8.21-8.19 (m, 1H), 8.18 (d, half of AB quartet, J = 8.8 Hz, 1H), 8.15 (dd, component of ABX system, J = 8.7, 2.5 Hz, 1H), 7.99-7.93 (m, 2H), 7.62 (t, J = 7.8 Hz, 1H), 7.16 (AB quartet, J_{AB} = 8.0 Hz, Δv_{AB} = 27.4 Hz, 4H), 6.86 (br t, J = 6.0 Hz, 1H), 4.54 (dd, J = 8.4, 3.0 Hz, 1H), 4.24 (dd, component of ABX system, J = 15.3, 6.0 Hz, 1H), 4.16 (dd, component of ABX system, J = 15.3, 5.7 Hz, 1H), 2.83 (septet, J = 6.9 Hz, 1H), 2.15- 2.04 (m, 1H), 2.01-1.86 (m, 3H), 1.16 (d, J = 6.9 Hz, 6H); 487.4 |
| 225 | 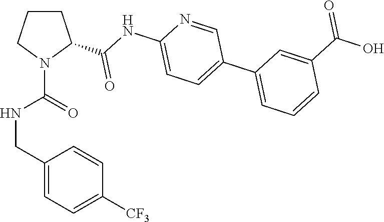 [SMILES: O=C(O)c1cccc(-c2ccc(NC(=O)[C@H]3CCCN3C(=O)NCc3ccc(C(F)(F)F)cc3)nc2)c1] | ¹H NMR, characteristic peaks: δ 10.58 (s, 1H), 8.69-8.65 (m, 1H), 8.21-8.19 (m, 1H), 8.17 (d, half of AB quartet, J = 8.8 Hz, 1H), 8.15 (dd, component of ABX system, J = 8.7, 2.5 Hz, 1H), 7.98-7.93 (m, 2H), 7.65 (d, J = 8.0 Hz, 2H), 7.61 (t, J = 7.8 Hz, 1H), 7.49 (d, J = 8.0 Hz, 2H), 7.04 (br t, J = 6.0 Hz, 1H), 4.56- 4.52 (m, 1H), 4.37 (dd, component of ABX system, J = 16.0, 6.0 Hz, 1H), 4.29 (dd, component of ABX system, J = 16.0, 5.8 Hz, 1H), 3.53-3.47 (m, 1H, assumed; partially obscured by water peak), 2.16-2.06 (m, 1H), 2.01-1.88 (m, 3H); 513.3 |
| 226 | 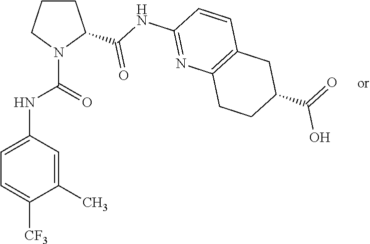 [SMILES: Cc1cc(NC(=O)N2CCC[C@@H]2C(=O)Nc2ccc3c(n2)CC[C@@H](C(=O)O)C3)ccc1C(F)(F)F] | ¹H NMR, characteristic peaks: δ 10.34 (s, 1H), 8.62 (s, 1H), 7.77 (d, J = 8.3 Hz, 1H), 7.59 (s, 1H), 7.51 (AB quartet, downfield doublet is broadened, J_{AB} = 8.8 Hz, Δv_{AB} = 20.2 Hz, 2H), 7.43 (d, J = 8.4 Hz, 1H), 4.62-4.55 (m, 1H), 3.67-3.60 (m, 1H, assumed; partially obscured by water peak), 2.85-2.68 (m, 3H), 2.35 (s, 3H), 2.19-2.13 (m, 1H), 2.12-2.05 (m, 1H), 2.01-1.94 (m, 1H), 1.94-1.84 (m, 2H), 1.81-1.72 (m, 1H); 491.4 |
| | 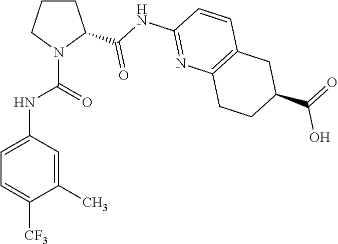 [SMILES: Cc1cc(NC(=O)N2CCC[C@@H]2C(=O)Nc2ccc3c(n2)CC[C@H](C(=O)O)C3)ccc1C(F)(F)F] | |
| | DIAST-1 (see footnote 48 in Table 2) | |
| 227 | 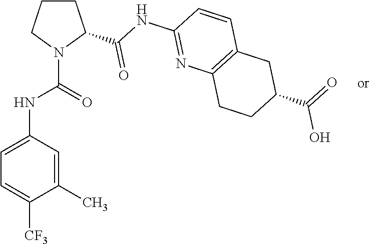 [SMILES: Cc1cc(NC(=O)N2CCC[C@@H]2C(=O)Nc2ccc3c(n2)CC[C@@H](C(=O)O)C3)ccc1C(F)(F)F] | ¹H NMR, characteristic peaks: δ 10.32 (s, 1H), 8.63 (s, 1H), 7.76 (d, J = 8.3 Hz, 1H), 7.59 (s, 1H), 7.51 (AB quartet, downfield doublet is broadened, J_{AB} = 8.7 Hz, Δv_{AB} = 20.7 Hz, 2H), 7.41 (d, J = 8.4 Hz, 1H), 4.63-4.54 (m, 1H), 3.67-3.60 (m, 1H), 3.56-3.49 (m, 1H, assumed; partially obscured by water peak), 2.35 (s, 3H), 2.21-2.12 (m, 1H), 2.08-2.01 (m, 1H), 2.01-1.94 (m, 1H), 1.94-1.85 (m, 2H), 1.79-1.69 (m, 1H); 491.4 |
| | 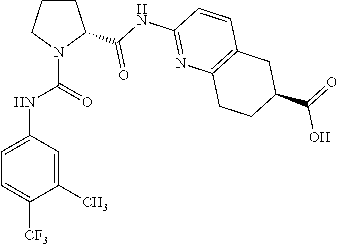 [SMILES: Cc1cc(NC(=O)N2CCC[C@@H]2C(=O)Nc2ccc3c(n2)CC[C@H](C(=O)O)C3)ccc1C(F)(F)F] | |
| | DIAST-2 (see footnote 48 in Table 2) | |
| 228 | 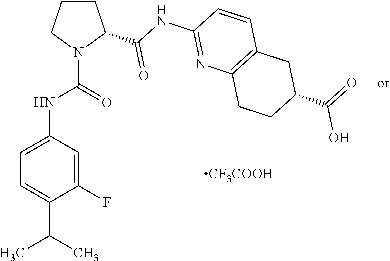 [SMILES: CC(C)c1ccc(NC(=O)N2CCC[C@@H]2C(=O)Nc2ccc3c(n2)CC[C@@H](C(=O)O)C3)cc1F ; "·CF3COOH"] | ¹H NMR, characteristic peaks: δ 10.31 (s, 1H), 8.38 (s, 1H), 7.79 (d, J = 8.4 Hz, 1H), 7.47 (d, J = 8.4 Hz, 1H), 7.38 (dd, J = 13.6, 2.1 Hz, 1H), 7.21 (dd, J = 8.5, 2.2 Hz, 1H), 7.15 (t, J = 8.7 Hz, 1H), 4.55 (dd, J = 8.4, 3.9 Hz, 1H), 3.64-3.57 (m, 1H, assumed; partially obscured by water peak), 3.06 (septet, J = 6.9 Hz, 1H), 2.88 (dd, component of ABX system, J = 16.6, 5.4 Hz, 1H), 2.84-2.75 (m, 3H), 1.17 (d, J = 6.9 Hz, 6H); 469.4 |
| | 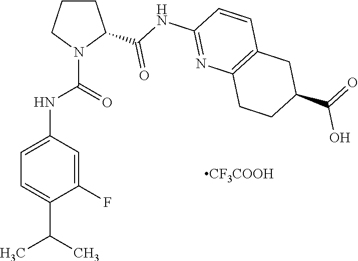 [SMILES: CC(C)c1ccc(NC(=O)N2CCC[C@@H]2C(=O)Nc2ccc3c(n2)CC[C@H](C(=O)O)C3)cc1F ; "·CF3COOH"] | |
| | DIAST-1 (see footnote 49 in Table 2) | |
| 229 | 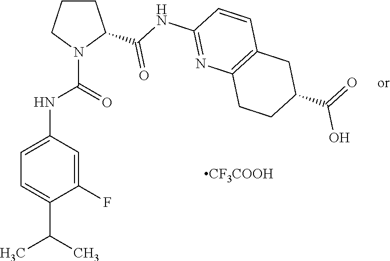 [SMILES: CC(C)c1ccc(NC(=O)N2CCC[C@@H]2C(=O)Nc2ccc3c(n2)CC[C@@H](C(=O)O)C3)cc1F ; "·CF3COOH"] | ¹H NMR, characteristic peaks: δ 10.31 (s, 1H), 8.38 (s, 1H), 7.79 (d, J = 8.4 Hz, 1H), 7.47 (d, J = 8.4 Hz, 1H), 7.37 (dd, J = 13.6, 2.1 Hz, 1H), 7.24-7.19 (m, 1H), 7.15 (t, J = 8.7 Hz, 1H), 4.55 (dd, J = 8.4, 3.9 Hz, 1H), 3.64-3.57 (m, 1H, assumed; partially obscured by water peak), 3.06 (septet, J = 6.9 Hz, 1H), 2.88 (dd, component of ABX system, J = 16.6, 5.5 Hz, 1H), 2.84-2.74 (m, 3H), 1.17 (d, J = 6.9 Hz, 6H); 469.4 |
| | 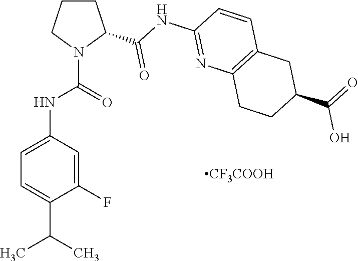 [SMILES: CC(C)c1ccc(NC(=O)N2CCC[C@@H]2C(=O)Nc2ccc3c(n2)CC[C@H](C(=O)O)C3)cc1F ; "·CF3COOH"] | |
| | DIAST-2 (see footnote 49 in Table 2) | |

¹Analytical conditions. Column: Waters Atlantis dC18, 4.6 × 50 mm, 5 μm; Mobile phase A: water containing 0.05% trifluoroacetic acid (v/v); Mobile phase B:
acetonitrile containing 0.05% trifluoroacetic acid (v/v); Gradient: 5.0% to 95% B, linear over 4.0 minutes, then 95% B for 1.0 minute; Flow rate: 2 mL/minute.

TABLE 2
Method of synthesis for Examples 8-15 and 39-229.

| Example | Method of synthesis; Non- |
|---|---|
| Number | commercial starting materials |
| 8 | Example 2; C2 |
| 9 | Example 2; C2 |
| 10 | C1¹ |
| 11 | Example 10²; C1 |
| 12 | Example 4³; C15 |
| 13 | Example 7 |
| 14 | Example 21; C2 |
| 15 | Example 6; C1 |
| 39 | Example 16; C15 |
| 40 | Example 21⁴; C2 |
| 41 | C1^{5,6} |
| 42 | Example 16; C15 |
| 43 | Example 21⁷; C15 |
| 44 | Example 36⁵; C11 |
| 45 | C2⁸ |
| 46 | C2⁸ |
| 47 | C2⁸ |
| 48 | C2⁸ |
| 49 | C2⁸ |
| 50 | C2⁸ |
| 51 | C2⁸ |
| 52 | C2⁸ |
| 53 | C2⁸ |
| 54 | C2⁸ |
| 55 | Example 19; C21 |
| 56 | Example 2; C2 |
| 57 | Example 21⁴; C2 |
| 58 | Example 21⁴; C2 |
| 59 | Example 19¹⁰; C35 |
| 60 | C22¹¹ |
| 61 | Example 25; C67 |
| 62 | Example 25; C67 |
| 63 | Example 19; C67 |
| 64 | Example 19; C67 |
| 65 | Example 6; C5 |
| 66 | C2⁸ |
| 67 | C2⁸ |
| 68 | C2⁸ |
| 69 | C2⁸ |
| 70 | C2⁸ |
| 71 | C2⁸ |
| 72 | Example 23; C35 |
| 73 | Example 19¹⁰; C35 |
| 74 | Example 19¹⁰; C35 |
| 75 | Example 4¹²; C1 |
| 76 | Example 3¹³; C1 |
| 77 | Example 3¹³; C1 |
| 78 | Example 3¹³; C1 |
| 79 | C2⁸ |
| 80 | C2⁸ |
| 81 | C2⁸ |
| 82 | C2⁸ |
| 83 | C2⁸ |
| 84 | C2⁸ |
| 85 | C2⁸ |
| 86 | C2⁸ |
| 87 | C2⁸ |
| 88 | C2⁸ |
| 89 | C2⁸ |
| 90 | C2⁸ |
| 91 | C2⁸ |
| 92 | C2⁸ |
| 93 | C2⁸ |
| 94 | C2⁸ |
| 95 | C2⁸ |
| 96 | C2⁸ |
| 97 | C2⁸ |
| 98 | C2⁸ |
| 99 | C2⁸ |
| 100 | C2⁸ |
| 101 | Example 19¹⁴; C35 |
| 102 | Example 19¹⁰; C35 |
| 103 | Example 19¹⁰; C35 |
| 104 | Example 24¹⁵; C11 |
| 105 | Example 104; C11 |
| 106 | Example 19¹⁰; C35 |
| 107 | Example 19¹⁰; C35 |
| 108 | Example 19¹⁰; C35 |
| 109 | Example 23; C35 |
| 110 | Example 22; C35 |
| 111 | Example 23; C35 |
| 112 | Example 23; C35 |
| 113 | Example 23; C35 |
| 114 | Example 23; C35 |
| 115 | Example 23; C35 |
| 116 | Example 23; C35 |
| 117 | Example 19¹⁴; C35 |
| 118 | C35¹⁶ |
| 119 | C35¹⁶ |
| 120 | C2¹⁷ |
| 121 | Example 25¹⁸ |
| 122 | Alternate Example 16; C8 |
| 123 | Example 24¹⁹; C13 |
| 124 | Example 19¹⁰; C35 |
| 125 | Example 19¹⁰; C35 |
| 126 | Example 19¹⁰; C35 |
| 127 | Example 19¹⁰; C35 |
| 128 | Example 19¹⁰; C35 |
| 129 | Example 19¹⁰; C35 |
| 130 | Example 19¹⁰; C35 |
| 131 | Example 19¹⁰; C35 |
| 132 | Example 19¹⁰; C35 |
| 133 | Example 19¹⁰; C35 |
| 134 | Example 19¹⁰; C35 |
| 135 | Example 19¹⁰; C35 |
| 136 | Example 19¹⁰; C35 |
| 137 | Example 19¹⁰; C35 |
| 138 | Example 19¹⁰; C35 |
| 139 | Example 19¹⁰; C35 |
| 140 | Example 19¹⁰; C35 |
| 141 | Example 19¹⁰; C35 |
| 142 | Example 19¹⁰; C35 |
| 143 | Example 19¹⁰; C35 |
| 144 | Example 19¹⁰; C35 |
| 145 | Example 19¹⁰; C35 |
| 146 | Example 19¹⁰; C35 |
| 147 | Example 26; C35 |
| 148 | C2⁸ |
| 149 | C35^{20,10} |
| 150 | Example 4²¹; C1 |
| 151 | Example 24^{22,23} |
| 152 | C42¹ |
| 153 | Example 25; C53 |
| 154 | Example 33²⁴ |
| 155 | Example 149; C35 |
| 156 | Example 25²² |
| 157 | Example 25; C67 |
| 158 | Example 30; C50 |
| 159 | Example 154 |
| 160 | Footnote 25 |
| 161 | C17¹ |
| 162 | Footnotes 26,¹ |
| 163 | Example 30; C30 |
| 164 | Example 30; C67 |
| 165 | Example 34²⁷; C11 |
| 166 | Example 34²⁷; C11 |
| 167 | Example 25; C26 |
| 168 | Example 25; C26 |
| 169 | Example 25; C26 |
| 170 | Example 30; C50 |
| 171 | Example 151 |
| 172 | Example 151 |
| 173 | Example 25²⁸ |
| 174 | Example 25; C21 |
| 175 | Example 30; C68 |
| 176 | Example 25²⁸ |
| 177 | Example 31; C53 |
| 178 | Example 31; C53 |
| 179 | Example 31; C53 |
| 180 | Example 17^{29,14} |
| 181 | Example 24¹⁰; C70 |
| 182 | Example 17^{29,14} |
| 183 | Example 30; C68 |
| 184 | Example 30³⁰; C68 |
| 185 | Example 38; C68 |
| 186 | Example 38³¹; C68 |
| 187 | Example 186; C68 |
| 188 | Example 24¹⁰; C70 |
| 189 | Example 186; C68 |
| 190 | Example 38; C68 |
| 191 | Example 38; C68 |
| 192 | Example 38; C68 |
| 193 | Example 24; C57 |
| 194 | Example 17¹⁴; C32 |
| 195 | Alternate Synthesis of Example |
| | 1³²; C8 |
| 196 | Example 17³³; C20 |
| 197 | Example 196 |
| 198 | Example 30³⁴; C30 |
| 199 | Example 24^{31,10}; C68 |
| 200 | Example 24^{35,10}; C68 |
| 201 | Example 17^{36,14} |
| 202 | Example 30³⁷; C68 |
| 203 | Example 30³⁷; C67 |
| 204 | Example 28; C17, C46 |
| 205 | Example 25; C26 |
| 206 | Example 25; C26 |
| 207 | Example 17; C53 |
| 208 | Example 196; C20 |
| 209 | Example 21³⁸; C17 |
| 210 | Example 19; C53 |
| 211 | Example 196; C20 |
| 212 | Example 19; C26 |
| 213 | Example 19³⁹; C26 |
| 214 | Example 5⁴⁵; C42, C46 |
| 215 | Alternate Synthesis of Example |
| | 22⁴⁰; C24 |
| 216 | Example 25³⁰; C26 |
| 217 | Footnotes 41, 42, 43 |
| 218 | Footnotes 41, 42, 43 |
| 219 | Example 5^{44,45}; C42 |
| 220 | Example 217⁴⁶ |
| 221 | Example 217⁴⁶ |
| 222 | Example 198³⁷; C30 |
| 223 | Example 25¹⁰; C70 |
| 224 | Example 25¹⁰; C70 |
| 225 | Example 25¹⁰; C70 |
| 226 | Footnotes 41, 47, 48 |
| 227 | Footnotes 41, 47, 48 |
| 228 | Example 226⁴⁹ |
| 229 | Example 226⁴⁹ |

¹The amide coupling was carried out using 2,4,6-tripropyl-1,3,5,2,4,6-trioxatriphosphinane 2,4,6-trioxide and 4-methylmorpholine.
²2-Chloro-8-methyl-6-nitroquinoline (see W. E. Blankenstein and J. D. Capps, J. Am. Chem. Soc. 1954, 76, 3211-3213) may be converted to the requisite 6-amino-8-methylquinoline-2-carboxylic acid using the method described by A. L Marzinzik et al., ChemMedChem 2015, 10, 1884-1891.
³Reaction of 4-(prop-1-en-2-yl)aniline with 4-nitrophenyl carbonochloridate in the presence of pyridine provided 4-nitrophenyl [4-(prop-1-en-2-yl)phenyl]carbamate, which was used to acylate C15 in the presence of N,N-diisopropylethylamine; this afforded the requisite (2R)-N²-(4-bromophenyl)-N¹-[4-(prop-1-en-2-yl)phenyl]pyrrolidine-1,2-dicarboxamide.
⁴The coupling was carried out using tripotassium phosphate rather than sodium carbonate.
⁵A racemic sample of 1-{[4-(propan-2-yl)phenyl]carbamoyl}proline was prepared by admixture of equal amounts of C1 and its enantiomer (synthesized in the same manner from L-proline).
⁶1-{[4-(Propan-2-yl)phenyl]carbamoyl}proline was coupled with 6-aminonaphthalene-2-carboxylic acid using 2,4,6-tripropyl-1,3,5,2,4,6-trioxatriphosphinane 2,4,6-trioxide, bromotripyrrolidinophosphonium hexafluorophosphate, and 4-methylmorpholine to afford Example 41.
⁷(2R)-N²-(4-Bromophenyl)-N¹-(4-chloro-3-methylphenyl)pyrrolidine-1,2-dicarboxamide was prepared from C15 using the method described in Step 2 of Example 16.
⁸Coupling of C2 with the appropriate chloro- or bromo-substituted phenol, amide, or carboxylic acid reactant was carried out using tetrakis(triphenylphosphine)palladium(0) and sodium carbonate.
¹⁰The final step was removal of the protecting group via treatment with trifluoroacetic acid.
¹¹Coupling with the appropriate boronic acid was carried out using [1,1′-bis(diphenylphosphino)ferrocene]dichloropalladium(II) and potassium carbonate.
¹²1-[3-(Dimethylamino)propyl]-3-ethylcarbodiimide hydrochloride-mediated reaction of C1 with 3-fluoro-4-(4,4,5,5-tetramethyl-1,3,2-dioxaborolan-2-yl)aniline afforded the requisite (2R)-N²-[3-fluoro-4-(4,4,5,5-tetramethyl-1,3,2-dioxaborolan-2-yl)phenyl]-N¹-[4-(propan-2-yl)phenyl]pyrrolidine-1,2-dicarboxamide.
¹³In this case, the carboxylic acid moiety on the boronic acid reactant was protected as its tert-butyl ester; the final deprotection step was therefore carried out with trifluoroacetic acid.
¹⁴The final step was removal of the protecting group via treatment with methanesulfonic acid.
¹⁵Reaction of (1R,2R,5S)-3-(tert-butoxycarbonyl)-3-azabicyclo[3.1.0]hexane-2-carboxylic acid with C11 was carried out using 1-[3-(dimethylamino)propyl]-3-ethylcarbodiimide hydrochloride to afford the requisite tert-butyl (1R,2R,5S)-2-{[4′-(tert-butoxycarbonyl)[1,1′-biphenyl]-4-yl]carbamoyl}-3-azabicyclo[3.1.0]hexane-3-carboxylate.
¹⁶Reaction of C35 with the appropriate isocyanate reactant was followed by deprotection with trifluoroacetic acid.
¹⁷Reaction of C2 with the appropriate bromo-substituted carboxylic acid reagent was carried out using [1,1′-bis(diphenylphosphino)ferrocene]dichloropalladium(II) and cesium carbonate.
¹⁸4-[5-(D-Prolylamino)pyrazin-2-yl]benzoic acid was synthesized using the method described for preparation of C38 in Example 24.
¹⁹In this case, the initial amide formation was carried out with O-(7-azabenzotriazol-1-yl)-N,N,N′,N′-tetramethyluronium hexafluorophosphate (HATU) and N,N-diisopropylethylamine.
²⁰Reaction of 4-cyclopropyl-3-fluoroaniline with 4-nitrophenyl carbonochloridate in the presence of pyridine, followed by addition of C35 and N,N-diisopropylethylamine, provided tert-butyl 4′-({1-[(4-cyclopropyl-3-fluorophenyl)carbamoyl]-D-prolyl}amino)[1,1′-biphenyl]-4-carboxylate.
²¹In this case, the initial amide coupling was carried out using 1-[3-(dimethylamino)propyl]-3-ethylcarbodiimide hydrochloride.
²²Coupling of (6-aminopyridin-3-yl)boronic acid and tert-butyl 4-bromo-3-methoxybenzoate was carried out using tetrakis(triphenylphosphine)palladium(0) and tripotassium phosphate to generate tert-butyl 4-(6-aminopyridin-3-yl)-3-methoxybenzoate.
²³tert-Butyl 4-(6-aminopyridin-3-yl)-3-methoxybenzoate (see footnote 22) was reacted with 1-(tert-butoxycarbonyl)-D-proline, O-(7-azabenzotriazol-1-yl)-N,N,N′,N′-tetramethyluronium hexafluorophosphate (HATU), and N,N-diisopropylethylamine. Removal of the protecting group with hydrogen chloride afforded the requisite 3-methoxy-4-[6-(D-prolylamino)pyridin-3-yl]benzoic acid.
²⁴Reaction of (4-aminophenyl)boronic acid and tert-butyl 3-bromobenzoate, in the presence of [1,1′-bis(diphenylphosphino)ferrocene]dichloropalladium(II) and potassium carbonate, provided tert-butyl 4′-amino[1,1′-biphenyl]-3-carboxylate; this material was coupled to (1R,2R,5S)-3-(tert-butoxycarbonyl)-3-azabicyclo[ 3.1.0]hexane-2-carboxylic acid using 1-[3-(dimethylamino)propyl]-3-ethylcarbodiimide hydrochloride to afford the requisite tert-butyl (1R,2R,5S)-2-{[3′-(tert-butoxycarbonyl)[1,1′-biphenyl]-4-yl]carbamoyl}-3-azabicyclo[3.1.0]hexane-3-carboxylate.
25. Coupling of 1-(tert-butoxycarbonyl)-D-proline and 6-bromo-5-methylpyridin-3-amine using 1-[3-(dimethylamino)propyl]-3-ethylcarbodiimide hydrochloride, followed by protecting group removal with methanesulfonic acid, provided N-(6-bromo-5-methylpyridin-3-yl)-D-prolinamide. This material was reacted with 4-cyclobutylaniline and bis(trichloromethyl) carbonate to afford (2R)-N²-(6-bromo-5-methylpyridin-3-yl)-N¹-(4-cyclobutylphenyl)pyrrolidine-1,2-dicarboxamide, which was coupled with 4-boronobenzoic acid, in the presence of [1,1′-bis(diphenylphosphino)ferrocene]dichloropalladium(II) and cesium carbonate, to yield Example 160.
26. The requisite 1-{[3-methyl-4-(propan-2-yl)phenyl]carbamoyl}-D-proline was prepared via reaction of 3-methyl-4-(propan-2-yl)aniline with D-proline, using 1,1′-carbonyldiimidazole and N,N-diisopropylethylamine.
²⁷Separation of the four individual isomers of penultimate intermediate tert-butyl 4′-[(3-ethyl-1-{[4-(propan-2-yl)phenyl]carbamoyl}-D-prolyl)amino][1,1′-biphenyl]-4-carboxylate was carried out using supercritical fluid chromatography [Column: Regis Technologies, (S,S)-Whelk-O 1, 25 × 250 mm, 10 μm; Mobile phase: 55:45 carbon dioxide/(ethanol containing 0.1% ammonium hydroxide); Flow rate: 80 mL/minute]
DIAST-1 - Retention time: 1.63 minutes; Analytical conditions [Column: Regis Technologies, (S,S)-Whelk-O 1, 4.6 × 100 mm, 5.0 μm; Mobile phase: 1:1 carbon dioxide/(ethanol containing 0.05% diethylamine); Flow rate: 2.5 mL/minute; Back pressure: 100 bar].
DIAST-2 - Retention time 2.32 minutes (Analytical conditions identical to those used for DIAST-1)
DIAST-3 - Retention time 3.45 minutes (Analytical conditions identical to those used for DIAST-1)
DIAST-4 - Retention time 4.12 minutes (Analytical conditions identical to those used for DIAST-1)
Comparison of the ¹H NMR spectra of these materials with those of C59 (DIAST-1), C60 (DIAST-2), C61 (DIAST-3), and C62 (DIAST-4) from Example 34 clearly indicated that DIAST-1 and DIAST-4 are enantiomers, of the same relative configuration as C59 and C62. Similarly, DIAST-2 and DIAST-3 are enantiomers of one another, and of the same relative configuration as C60 and C61.
Example 165 was synthesized from DIAST-1, and Example 166 was synthesized from DIAST-2.
²⁸The requisite 3-methyl-4-[6-(D-prolylamino)pyridin-3-yl]benzoic acid was prepared using the general method described in footnote 22.
²⁹Coupling of tert-butyl 6-bromopyridine-3-carboxylate and (4-aminophenyl)boronic acid, using [1,1′-bis(diphenylphosphino)ferrocene]dichloropalladium(II) and potassium carbonate, provided tert-butyl 6-(4-aminophenyl)pyridine-3-carboxylate. This material was converted to the requisite tert-butyl 6-[4-(D-prolylamino)phenyl]pyridine-3-carboxylate according to the procedure outlined for synthesis of C35 in Example 23.
³⁰5-Bromo-4-methylpyridin-2-amine and 4,4,5,5-tetramethyl-2-(prop-1-en-2-yl)-1,3,2-dioxaborolane were coupled using tetrakis(triphenylphosphine)palladium(0) and potassium carbonate. Hydrogenation of the product over palladium on carbon afforded the requisite 4-methyl-5-(propan-2-yl)pyridin-2-amine.
³¹Reaction between C68 and tert-butyl 2-fluoro-4-(4,4,5,5-tetramethyl-1,3,2-dioxaborolan-2-yl)benzoate was catalyzed by mesylate[(di(1-adamantyl)-n-butylphosphine)-2-(2′-amino-1,1′-biphenyl)]palladium(II) (cataCXium ® A Pd G3), in the presence of sodium carbonate, affording tert-butyl (2R)-2-({5-[4-(tert-butoxycarbonyl)-3-fluorophenyl]pyridin-2-yl}carbamoyl)pyrrolidine-1-carboxylate.
³²Amide couple of D-proline and 1-[4-(trifluoromethyl)phenyl]methanamine with bis(trichloromethyl) carbonate and 4-(dimethylamino)pyridine provided 1-({[4-(trifluoromethyl)phenyl]methyl}carbamoyl)-D-proline.
³³In this case, removal of the protecting group from C20 provided tert-butyl 5-[4-(D-prolylamino)phenyl]pyridine-2-carboxylate, so after the bis(trichloromethyl) carbonate step, the product was deprotected with methanesulfonic acid.
³⁴Reaction of C30 and 4-boronobenzoic acid, mediated by tetrakis(triphenylphosphine)palladium(0) and sodium carbonate, was followed by deprotection with hydrogen chloride, affording the requisite 4-[5-(D-prolylamino)pyridin-2-yl]benzoic acid.
³⁵Coupling of C68 with tert-butyl 2-methyl-4-(4,4,5,5-tetramethyl-1,3,2-dioxaborolan-2-yl)benzoate, using [1,1′-bis(diphenylphosphino)ferrocene]dichloropalladium(II) and sodium carbonate, provided the requisite tert-butyl (2R)-2-({5-[ 4-(tert-butoxycarbonyl)-3-methylphenyl]pyridin-2-yl}carbamoyl)pyrrolidine-1-carboxylate. Subsequent deprotection using hydrochloric acid in ethyl acetate provided the requisite tert-butyl 2-methyl-4-[6-(D-prolylamino)pyridin-3-yl]benzoate.
³⁶Reaction between 5-(4,4,5,5-tetramethyl-1,3,2-dioxaborolan-2-yl)pyridin-2-amine and tert-butyl 5-bromopyridine-2-carboxylate was catalyzed by mesylate[(di(1-adamantyl)-n-butylphosphine)-2-(2′-amino-1,1′-biphenyl)]palladium(II) (cataCXium ® A Pd G3), in the presence of sodium carbonate, affording tert-butyl 6′-amino[3,3′-bipyridine]-6-carboxylate. This material was subjected to amide formation with 1-(tert-butoxycarbonyl)-D-proline, mediated by 1-[3-(dimethylamino)propyl]-3-ethylcarbodiimide hydrochloride, to afford tert-butyl 6′-{[1-(tert-butoxycarbonyl)-D-prolyl]amino}[3,3′-bipyridine]-6-carboxylate. Deprotection with hydrogen chloride provided the requisite tert-butyl 6′-(D-prolylamino)[3,3′-bipyridine]-6-carboxylate.
³⁷Reaction of 2-chloro-4-methyl-5-(trifluoromethyl)pyridine and 1-(4-methoxyphenyl)methanamine in the presence of N,N-diisopropylethylamine, followed by protecting group cleavage with methanesulfonic acid, provided 4-methyl-5-(trifluoromethyl)pyridin-2-amine. Conversion to the requisite 2-isocyanato-4-methyl-5-(trifluoromethyl)pyridine was carried out using 1,1′-carbonyldiimidazole and triethylamine.
³⁸Amide coupling of C17 and 4-(4,4,5,5-tetramethyl-1,3,2-dioxaborolan-2-yl)aniline, using 1-[3-(dimethylamino)propyl]-3-ethylcarbodiimide hydrochloride, provided the requisite (2R)-N²-[4-(4,4,5,5-tetramethyl-1,3,2-dioxaborolan-2-yl)phenyl]-N¹-[4-(trifluoromethyl)phenyl]pyrrolidine-1,2-dicarboxamide.
³⁹6-Bromo-5-fluoropyridin-3-amine and 4,4,5,5-tetramethyl-2-(prop-1-en-2-yl)-1,3,2-dioxaborolane were coupled using tetrakis(triphenylphosphine)palladium(0) and potassium carbonate. Hydrogenation of the product over palladium on carbon afforded the requisite 5-fluoro-6-(propan-2-yl)pyridin-3-amine.
⁴⁰Reaction of D-proline with 3-fluoro-4-(trifluoromethyl)aniline, 1,1′-carbonyldiimidazole, and 4-methylmorpholine provided 1-{[3-fluoro-4-(trifluoromethyl)phenyl]carbamoyl}-D-proline.
41. Treatment of 2-aminoquinoline-6-carboxylic acid with acetic anhydride and acetic acid afforded 2-acetamidoquinoline-6-carboxylic acid; this material was esterified using iodomethane and potassium carbonate, whereupon hydrogenation over platinum(IV) oxide at 65° C. in trifluoroacetic acid provided methyl 2-acetamido-5,6,7,8-tetrahydroquinoline-6-carboxylate. Ester exchange via treatment with sulfuric acid in butan-1-ol yielded butyl 2-amino-5,6,7,8-tetrahydroquinoline-6-carboxylate.
42. Reaction of 1-(tert-butoxycarbonyl)-D-proline with butyl 2-amino-5,6,7,8-tetrahydroquinoline-6-carboxylate using 1-[3-(dimethylamino)propyl]-3-ethylcarbodiimide hydrochloride and N,N-diisopropylethylamine, followed by treatment with trifluoroacetic acid, provided butyl 2-(D-prolylamino)-5,6,7,8-tetrahydroquinoline-6-carboxylate. This material was reacted with 1-isocyanato-4-(trifluoromethyl)benzene, in the presence of 4-methylmorpholine, whereupon the product was subjected to ester hydrolysis with lithium hydroxide, affording a diastereomeric mixture of Examples 217 and 218.
43. The product was separated into its component diastereomers using supercritical fluid chromatography (Column: Chiral Technologies Chiralpak AS-H, 21 × 250 mm, 5 μm; Mobile phase: 4:1 carbon dioxide/methanol; Flow rate: 75 mL/minute; Back pressure: 120 bar).
The first-eluting diastereomer was designated as Example 217, and the second-eluting diastereomer as Example 218. On analytical supercritical fluid chromatography (Column: Chiral Technologies Chiralpak AS-H, 4.6 × 100 mm, 5 μm; Mobile phase: 3:1 carbon dioxide/methanol; Flow rate: 1.5 mL/minute; Back pressure: 120 bar), Example 217 exhibited a retention time of 5.51 minutes. Example 218 had a retention time of 6.97 minutes under the same conditions.
⁴⁴Coupling of [4-(tert-butoxycarbonyl)phenyl]boronic acid and 4-bromo-3-methylaniline was carried out using tetrakis(triphenylphosphine)palladium(0) and tripotassium phosphate to provide the requisite tert-butyl 4′-amino-2′-methyl[1,1′-biphenyl]-4-carboxylate.
⁴⁵The final step was removal of the protecting group via treatment with hydrogen chloride.
⁴⁶The product was separated into its component diastereomers using supercritical fluid chromatography (Column: Chiral Technologies Chiralpak AS-H, 21 × 250 mm, 5 μm; Mobile phase: 4:1 carbon dioxide/methanol; Flow rate: 75 mL/minute; Back pressure: 120 bar).
The first-eluting diastereomer was designated as Example 220, and the second-eluting diastereomer as Example 221. On analytical supercritical fluid chromatography (Column: Chiral Technologies Chiralpak AS-H, 4.6 × 100 mm, 5 μm; Mobile phase: 3:1 carbon dioxide/methanol; Flow rate: 1.5 mL/minute; Back pressure: 120 bar), Example 220 exhibited a retention time of 5.6 minutes. Example 221 had a retention time of 7.22 minutes under the same conditions.
47 Reaction of butyl 2-(D-prolylamino)-5,6,7,8-tetrahydroquinoline-6-carboxylate (see footnote 42) with 3-methyl-4-(trifluoromethyl)aniline and bis(trichloromethyl) carbonate in the presence of 4-methylmorpholine and 4-(dimethylamino)pyridine provided butyl 2-[(1-{[3-methyl-4-(trifluoromethyl)phenyl]carbamoyl}-D-prolyl)amino]-5,6,7,8-tetrahydroquinoline-6-carboxylate. Subsequent ester hydrolysis using lithium hydroxide afforded a diastereomeric mixture of Examples 226 and 227.
48 The product was separated into its component diastereomers using supercritical fluid chromatography (Column: Chiral Technologies Chiralpak IA, 21 × 250 mm, 5 μm; Mobile phase: 3:1 carbon dioxide/methanol; Flow rate: 75 mL/minute; Back pressure: 120 bar).
The first-eluting diastereomer was designated as Example 226, and the second-eluting diastereomer as Example 227. On analytical supercritical fluid chromatography (Column: Chiral Technologies Chiralpak IA, 4.6 × 100 mm, 5 μm; Mobile phase: 65:35 carbon dioxide/methanol; Flow rate: 1.5 mL/minute; Back pressure: 120 bar), Example 226 exhibited a retention time of 6.69 minutes. Example 227 had a retention time of 7.7 minutes under the same conditions.
⁴⁹The product was separated into its component diastereomers using supercritical fluid chromatography [Column: Chiral Technologies Chiralpak IH, 21 × 250 mm, 5 μm; Mobile phase: 7:3 carbon dioxide/(0.1% trifluoroacetic acid in a 1:1:1 mixture of propan-2-ol/ethanol/methanol); Flow rate: 75 mL/minute; Back pressure: 120 bar].

The first-eluting diastereomer was designated as Example 228, and the second-eluting diastereomer as Example 229. On analytical supercritical fluid chromatography [Column: Chiral Technologies Chiralpak IH, 4.6×100 mm, 5 μm; Mobile phase: 65:35 carbon dioxide/(0.1% trifluoroacetic acid in a 1:1:1 mixture of propan-2-ol/ethanol/methanol); Flow rate: 1.5 mL/minute; Back pressure: 120 bar], Example 228 exhibited a retention time of 5.24 minutes. Example 229 had a retention time of 6.01 minutes under the same conditions.

### Example AA. Functional In Vitro GIPR Antagonist Potency Assay

The functional in vitro antagonist potency for test compounds was determined by monitoring intracellular cyclic adenosine monophosphate (cAMP) levels in Chinese hamster ovary (CHO)—K1 cells stably expressing the human Glucose-dependent Insulinotropic Polypeptide Receptor (hGIPR). Following agonist activation, hGIPR associates with the G-protein complex causing the Gαs subunit to exchange bound guanosine diphosphate (GDP) for guanosine triphosphate (GTP), followed by dissociation of the Gαs-GTP complex. The activated Gas subunit can couple to downstream effectors to regulate the levels of second messengers or cAMP within the cell. Thereby, determination of intracellular cAMP levels allows for pharmacological characterization. Intracellular cAMP levels are quantitated using a homogenous assay utilizing the Homogeneous Time Resolved Fluorescence (HTRF) technology from Perkin Elmer. The method is a competitive immunoassay between native cAMP produced by the cells and cAMP labelled with the acceptor dye, d2. The two entities compete for binding to a monoclonal anti-cAMP antibody labeled with cryptate. The specific signal is inversely proportional to the concentration of cAMP in the cells.

Test compounds were solubilized to a concentration of 30 mM in 100% dimethyl sulfoxide (DMSO). An 11-point dilution series using 1 in 3.162-fold serial dilutions was created in 100% DMSO with a top concentration of 8 mM. The serially diluted compound was spotted with an Echo Acoustic liquid handler (Beckman Coulter) into a 384-well assay plate (Corning, Cat No. 3824) at 50 nL/well with duplicate points at each concentration, at a 200× final assay concentration (FAC). The final compound concentration range in the assay was 40 μM to 400 μM, with a final DMSO concentration of 0.5%.

Frozen assay-ready vials (at 1×10⁷ cells/vial) of CHO-K1 cells stably expressing the Gs-coupled human GIPR receptor (Eurofins, DiscoverX, Cat No. 95-0146C2) were thawed, counted, and resuspended in assay buffer consisting of Hank's Balanced Salt Solution (HBSS, Lonza Cat No. 10-527) containing 20 mM (4-(2-hydroxyethyl)-1-piperazineethanesulfonic acid (HEPES, Lonza, Cat No. 17-737E), 0.1% bovine serum albumin (BSA, Sigma, Cat No. A7979), and 200 μM 3-isobutyl-1-methylxanthine (IBMX, Sigma, Cat No. 15879) at a density of 4×10⁵ cells/mL. Cells were added to assay plate (5 μL/well of 4×10⁵ cells/mL stock for 2,000 cells/well final) containing 50 nL of 200× FAC test compound, and incubated at 37° C. (95% O₂: 5% CO₂) for 2 hours, with micro-clime lids (Labcyte, Cat No. LLS-0310). Following the 2-hour cell and compound incubation, a stimulation mix comprised of hGIPR agonist human glucose-dependent insulinotropic polypeptide (hGIP, full length, Sigma Cat No. G2269) in assay buffer/0.1% DMSO was added to the assay plate (5 μL/well) at an estimated EC₈₀ FAC (based on previous hGIP agonist curves) and incubated for another 30 minutes with micro-clime lids at 37° C. (95% O₂: 5% CO₂), after which intracellular cAMP levels were quantified as per Perkin Elmer's protocol (5 μL of d2 and then 5 μL cryptate, incubated for 1 hour at room temperature). Emission spectra of samples were measured on a Pherastar plate reader (BMG Labtech Inc) using a HTRF protocol (excitation, 320 nm; emission, 665 nm/620 nm).

hGIP EC₅₀ was determined daily by incubating cells (5 μL/well of 4×10⁵ cells/mL stock, for 2,000 cells/well final) with 50 nL 100% DMSO for 2 hours at 37° C. (95% O₂: 5% CO₂), with a micro-clime lid. Following the 2-hour cell and DMSO incubation, a hGIP concentration response curve at 2× FAC (12-point curve using 1 in 3 serial dilutions, with triplicate points at each concentration, 100 nM final top concentration) in assay buffer/1% DMSO was added (5 μL/well) and incubated for a further 30 minutes with a micro-clime lid at 37° C. (95% O₂: 5% CO₂), after which intracellular cAMP levels were quantified and samples measured as described previously. Experiments passed quality control if the agonist concentration used for stimulation fell between the on-the-day EC₅₀-EC₉₀.

Data were analyzed using the ratio of fluorescence intensity at 620 and 665 nm for each well, extrapolated from the cAMP standard curve to express data as nanomolar (nM) cAMP for each well. Data expressed as nM cAMP were then normalized to control wells using ActivityBase (IDBS data management software). Zero percent effect (ZPE) was defined as nM cAMP generated from the hGIP stimulation mix, while 100% effect, or one hundred percent effect (HPE), was defined as nM cAMP generated from the combined effects of hGIP simulation mix+antagonism by 80 μM of (−)-3-(6-(2-methyl-1-(4′-(trifluoromethyl)biphenyl-4-yl)propylamino)nicotinamido)propanoic acid as GIPR antagonist. The concentration and % effect values for each compound were plotted by ActivityBase using a four-parameter logistic dose response equation, and the concentration required for 50% inhibition (IC₅₀) was determined.

Table 3 lists biological activities (IC₅₀ values) and compound names for Examples 1-229.

TABLE 3
Biological activity and Compound name for Examples 1-229.

| | | hGIPR | |
|---|---|---|---|
| | | antagonist | |
| | hGIPR | IC₅₀ | |
| | antagonist | replicate | |
| Example | IC₅₀ (nM)¹ | count | Compound Name |
| 1² | 16 | 12 | ammonium 5-{4-[(1-{[4-(propan-2- |
| | | | yl)phenyl]carbamoyl}-D-prolyl)amino]phenyl}pyridine- |
| | | | 2-carboxylate |
| 2 | 74 | 3 | 6-{4-[(1-{[4-(propan-2-yl)phenyl]carbamoyl}-D- |
| | | | prolyl)amino]phenyl}pyridine-3-carboxylic acid |
| 3 | 17 | 6 | 4′-[(1-{[4-(propan-2-yl)phenyl]carbamoyl}-D- |
| | | | prolyl)amino][1,1′-biphenyl]-3-carboxylic acid |
| 4 | 4.1 | 14 | 4′-[(1-{[4-(propan-2-yl)phenyl]carbamoyl}-D- |
| | | | prolyl)amino][1,1′-biphenyl]-4-carboxylic acid |
| 5² | 9.1 | 6 | ammonium 4-{6-[(1-{[4-(propan-2- |
| | | | yl)phenyl]carbamoyl}-D-prolyl)amino]pyridin-3- |
| | | | yl}benzoate |
| 6 | 6.5 | 5 | 3′-fluoro-4′-[(1-{[4-(propan-2-yl)phenyl]carbamoyl}-D- |
| | | | prolyl)amino][1,1′-biphenyl]-4-carboxylic acid |
| 7 | 8.1 | 5 | ammonium 4′-({1-[(4-cyclopropylphenyl)carbamoyl]-D- |
| | | | prolyl}amino)[1,1′-biphenyl]-4-carboxylate |
| 8² | 150 | 5 | ammonium 2-{4-[(1-{[4-(propan-2- |
| | | | yl)phenyl]carbamoyl}-D- |
| | | | prolyl)amino]phenyl}pyrimidine-5-carboxylate |
| 9 | 130 | 3 | ammonium 6-{4-[(1-{[4-(propan-2- |
| | | | yl)phenyl]carbamoyl}-D-prolyl)amino]phenyl}pyridine- |
| | | | 2-carboxylate |
| 10² | 67 | 3 | 6-[(1-{[4-(propan-2-yl)phenyl]carbamoyl}-D- |
| | | | prolyl)amino]naphthalene-2-carboxylic acid |
| 11² | 180 | 3 | 8-methyl-6-[(1-{[4-(propan-2-yl)phenyl]carbamoyl}-D- |
| | | | prolyl)amino]quinoline-2-carboxylic acid, |
| | | | trifluoroacetate salt |
| 12 | 10 | 5 | 4′-[(1-{[4-(prop-1-en-2-yl)phenyl]carbamoyl}-D- |
| | | | prolyl)amino][1,1′-biphenyl]-4-carboxylic acid |
| 13 | 12 | 9 | ammonium 4′-({1-[(4-chlorophenyl)carbamoyl]-D- |
| | | | prolyl}amino)[1,1′-biphenyl]-4-carboxylate |
| 14 | 88 | 3 | ammonium 4-{4-[(1-{[4-(propan-2- |
| | | | yl)phenyl]carbamoyl}-D-prolyl)amino]phenyl}pyridine- |
| | | | 2-carboxylate |
| 15 | 8.1 | 6 | 3′,5′-difluoro-4′-[(1-{[4-(propan-2- |
| | | | yl)phenyl]carbamoyl}-D-prolyl)amino][1,1′-biphenyl]-4- |
| | | | carboxylic acid |
| 16 | 3.7 | 7 | ammonium 4′-[(1-{[4- |
| | | | (trifluoromethyl)phenyl]carbamoyl}-D- |
| | | | prolyl)amino][1,1′-biphenyl]-4-carboxylate |
| 17 | 14 | 9 | 5-{4-[(1-{[3-methyl-4- |
| | | | (trifluoromethyl)phenyl]carbamoyl}-D- |
| | | | prolyl)amino]phenyl}pyridine-2-carboxylic acid |
| 18 | 15 | 9 | 4-{5-[(1-{[4-(propan-2-yl)phenyl]carbamoyl}-D- |
| | | | prolyl)amino]pyridin-2-yl}benzoic acid |
| 19 | 11 | 10 | 5-{4-[(1-{[3-methyl-4-(propan-2-yl)phenyl]carbamoyl}- |
| | | | D-prolyl)amino]phenyl}pyridine-2-carboxylic acid |
| 20 | 12 | 14 | 6-methyl-5-{4-[(1-{[4-(propan-2-yl)phenyl]carbamoyl}- |
| | | | D-prolyl)amino]phenyl}pyridine-2-carboxylic acid |
| 21² | 11 | 19 | 3-methoxy-4′-[(1-{[4-(propan-2-yl)phenyl]carbamoyl}- |
| | | | D-prolyl)amino][1,1′-biphenyl]-4-carboxylic acid |
| 22 | 62 | 10 | 4-{5-[(1-{[4-(trifluoromethyl)phenyl]carbamoyl}-D- |
| | | | prolyl)amino]pyridin-2-yl}benzoic acid |
| 23 | 2.7 | 5 | 4′-({1-[(4-cyclobutylphenyl)carbamoyl]-D- |
| | | | prolyl}amino)[1,1′-biphenyl]-4-carboxylic acid |
| 24 | 110 | 4 | 4-{5-fluoro-6-[(1-{[4- |
| | | | (trifluoromethyl)phenyl]carbamoyl}-D- |
| | | | prolyl)amino]pyridin-3-yl}benzoic acid |
| 25 | 86 | 4 | 4-{5-fluoro-6-[(1-{[4-(propan-2-yl)phenyl]carbamoyl}- |
| | | | D-prolyl)amino]pyridin-3-yl}benzoic acid |
| 26 | 2.1 | 4 | 4′-[(1-{[4-cyclopropyl-3- |
| | | | (trifluoromethyl)phenyl]carbamoyl}-D- |
| | | | prolyl)amino][1,1′-biphenyl]-4-carboxylic acid |
| 27² | 4.8 | 18 | 5-{4-[(1-{[3-fluoro-4-(propan-2-yl)phenyl]carbamoyl}- |
| | | | D-prolyl)amino]phenyl}-6-methylpyridine-2-carboxylic |
| | | | acid |
| 28² | 10 | 16 | 3-fluoro-4′-[(1-{[4-(propan-2-yl)phenyl]carbamoyl}-D- |
| | | | prolyl)amino][1,1′-biphenyl]-4-carboxylic acid |
| 29 | 17 | 14 | 2-methoxy-4′-[(1-{[4-(propan-2-yl)phenyl]carbamoyl}- |
| | | | D-prolyl)amino][1,1′-biphenyl]-4-carboxylic acid |
| 30 | 5.7 | 10 | 3-methoxy-4′-[(1-{[3-methyl-4-(propan-2- |
| | | | yl)phenyl]carbamoyl}-D-prolyl)amino][1,1′-biphenyl]-4- |
| | | | carboxylic acid |
| 31 | 7.8 | 10 | 4-{6-[(1-{[4-(trifluoromethyl)phenyl]carbamoyl}-D- |
| | | | prolyl)amino]pyridin-3-yl}benzoic acid |
| 32 | 6.1 | 3 | 4′-[(1-{[3-fluoro-4-(propan-2-yl)phenyl]carbamoyl}-D- |
| | | | prolyl)amino]-3-methoxy[1,1′-biphenyl]-4-carboxylic |
| | | | acid |
| 33 | 6.3 | 6 | 4-{3-fluoro-5-[(1-{[4-(propan-2-yl)phenyl]carbamoyl}- |
| | | | D-prolyl)amino]pyridin-2-yl}benzoic acid |
| 34 | 10 | 5 | 4′-{[(3S)-3-methyl-1-{[4-(propan-2- |
| | | | yl)phenyl]carbamoyl}-D-prolyl]amino}[1,1′-biphenyl]-4- |
| | | | carboxylic acid |
| | | | or |
| | | | 4′-{[(3R)-3-methyl-1-{[4-(propan-2- |
| | | | yl)phenyl]carbamoyl}-L-prolyl]amino}[1,1′-biphenyl]-4- |
| | | | carboxylic acid |
| 35 | 4.6 | 4 | 4′-{[(3R)-3-methyl-1-{[4-(propan-2- |
| | | | yl)phenyl]carbamoyl}-D-prolyl]amino}[1,1′-biphenyl]-4- |
| | | | carboxylic acid |
| | | | or |
| | | | 4′-{[(3S)-3-methyl-1-{[4-(propan-2- |
| | | | yl)phenyl]carbamoyl}-L-prolyl]amino}[1,1′-biphenyl]-4- |
| | | | carboxylic acid |
| 36 | 46 | 3 | 4′-{[(4R)-4-methoxy-1-{[4-(propan-2- |
| | | | yl)phenyl]carbamoyl}-D-prolyl]amino}[1,1′-biphenyl]-4- |
| | | | carboxylic acid |
| 37 | 58 | 3 | 4′-{[1-({(1S)-1-[4-(propan-2- |
| | | | yl)phenyl]ethyl}carbamoyl)-D-prolyl]amino}[1,1′- |
| | | | biphenyl]-4-carboxylic acid |
| 38 | 17 | 3 | 3-[6-({1-[(4-cyclobutylphenyl)carbamoyl]-D- |
| | | | prolyl}amino)pyridin-3-yl]benzoic acid |
| 39 | 3.7 | 3 | ammonium 4′-[(1-{[3-methyl-4-(propan-2- |
| | | | yl)phenyl]carbamoyl}-D-prolyl)amino][1,1′-biphenyl]-4- |
| | | | carboxylate |
| 40 | 7.3 | 7 | 3-methyl-4′-[(1-{[4-(propan-2-yl)phenyl]carbamoyl}-D- |
| | | | prolyl)amino][1,1′-biphenyl]-4-carboxylic acid |
| 41 | 160 | 3 | 6-[(1-{[4-(propan-2- |
| | | | yl)phenyl]carbamoyl}prolyl)amino]naphthalene-2- |
| | | | carboxylic acid |
| 42² | 15 | 5 | ammonium 4′-{[1-({4-[1- |
| | | | (trifluoromethyl)cyclopropyl]phenyl}carbamoyl)-D- |
| | | | prolyl]amino}[1,1′-biphenyl]-4-carboxylate |
| 43 | 49 | 3 | ammonium 4′-({1-[(4-chloro-3- |
| | | | methylphenyl)carbamoyl]-D-prolyl}amino)[1,1′- |
| | | | biphenyl]-4-carboxylate |
| 44 | 6.8 | 4 | 4′-[(1-{[4-(propan-2- |
| | | | yl)phenyl]carbamoyl}prolyl)amino][1,1′-biphenyl]-4- |
| | | | carboxylic acid |
| 45 | 540 | 5 | 4,6-difluoro-4′-[(1-{[4-(propan-2-yl)phenyl]carbamoyl}- |
| | | | D-prolyl)amino][1,1′-biphenyl]-3-carboxylic acid |
| 46 | 160 | 3 | (2R)-N²-(4′-hydroxy[1,1′-biphenyl]-4-yl)-N¹-[4-(propan- |
| | | | 2-yl)phenyl]pyrrolidine-1,2-dicarboxamide |
| 47 | 47 | 3 | (2R)-N²-(2′,5′-difluoro-4′-hydroxy[1,1′-biphenyl]-4-yl)- |
| | | | N¹-[4-(propan-2-yl)phenyl]pyrrolidine-1,2- |
| | | | dicarboxamide |
| 48 | 23 | 3 | 2,6-difluoro-4′-[(1-{[4-(propan-2-yl)phenyl]carbamoyl}- |
| | | | D-prolyl)amino][1,1′-biphenyl]-4-carboxylic acid |
| 49 | 14 | 4 | 4′-[(1-{[4-(propan-2-yl)phenyl]carbamoyl}-D- |
| | | | prolyl)amino]-2-(trifluoromethyl)[1,1′-biphenyl]-4- |
| | | | carboxylic acid |
| 50 | 83 | 2 | 5-methyl-4′-[(1-{[4-(propan-2-yl)phenyl]carbamoyl}-D- |
| | | | prolyl)amino][1,1′-biphenyl]-3-carboxylic acid |
| 51 | 280 | 4 | (2R)-N²-(2′-fluoro-4′-hydroxy[1,1′-biphenyl]-4-yl)-N¹-[4- |
| | | | (propan-2-yl)phenyl]pyrrolidine-1,2-dicarboxamide |
| 52 | 120 | 3 | (2R)-N²-(4′-carbamoyl-3′-methyl[1,1′-biphenyl]-4-yl)- |
| | | | N¹-[4-(propan-2-yl)phenyl]pyrrolidine-1,2- |
| | | | dicarboxamide |
| 53 | 15 | 1 | 3,5-dimethyl-4′-[(1-{[4-(propan-2- |
| | | | yl)phenyl]carbamoyl}-D-prolyl)amino][1,1′-biphenyl]-4- |
| | | | carboxylic acid |
| 54 | 140 | 4 | (2R)-N²-(3′-fluoro-4′-hydroxy[1,1′-biphenyl]-4-yl)-N¹-[4- |
| | | | (propan-2-yl)phenyl]pyrrolidine-1,2-dicarboxamide |
| 55 | 7.6 | 3 | 5-{4-[(1-{[3-chloro-4- |
| | | | (trifluoromethyl)phenyl]carbamoyl}-D- |
| | | | prolyl)amino]phenyl}pyridine-2-carboxylic acid |
| 56 | 6.3 | 7 | 2-fluoro-4′-[(1-{[4-(propan-2-yl)phenyl]carbamoyl}-D- |
| | | | prolyl)amino][1,1′-biphenyl]-4-carboxylic acid |
| 57 | 65 | 3 | 4-methyl-5-{4-[(1-{[4-(propan-2-yl)phenyl]carbamoyl}- |
| | | | D-prolyl)amino]phenyl}pyridine-2-carboxylic acid |
| 58 | 50 | 3 | 3-methyl-5-{4-[(1-{[4-(propan-2-yl)phenyl]carbamoyl}- |
| | | | D-prolyl)amino]phenyl}pyridine-2-carboxylic acid |
| 59 | 35 | 3 | 4′-({1-[(3,5-dichlorophenyl)carbamoyl]-D- |
| | | | prolyl}amino)[1,1′-biphenyl]-4-carboxylic acid |
| 60 | 21 | 4 | 3-methyl-4-{5-[(1-{[4-(propan-2-yl)phenyl]carbamoyl}- |
| | | | D-prolyl)amino]pyridin-2-yl}benzoic acid |
| 61 | 150 | 4 | 4′-[(1-{[5-(propan-2-yl)pyridin-2-yl]carbamoyl}-D- |
| | | | prolyl)amino][1,1′-biphenyl]-4-carboxylic acid |
| 62 | 110 | 5 | 4′-[(1-{[6-(propan-2-yl)pyridin-3-yl]carbamoyl}-D- |
| | | | prolyl)amino][1,1′-biphenyl]-4-carboxylic acid |
| 63 | 6.2 | 5 | 4′-({1-[(4-bromophenyl)carbamoyl]-D- |
| | | | prolyl}amino)[1,1′-biphenyl]-4-carboxylic acid |
| 64 | 2.9 | 10 | 4′-[(1-{[3-fluoro-4-(propan-2-yl)phenyl]carbamoyl}-D- |
| | | | prolyl)amino][1,1′-biphenyl]-4-carboxylic acid |
| 65 | 13 | 4 | 5-{3-fluoro-4-[(1-{[4-(propan-2-yl)phenyl]carbamoyl}- |
| | | | D-prolyl)amino]phenyl}pyridine-2-carboxylic acid |
| 66 | 390 | 54 | (2R)-N²-(2′-fluoro-3′-hydroxy[1,1′-biphenyl]-4-yl)-N¹-[4- |
| | | | (propan-2-yl)phenyl]pyrrolidine-1,2-dicarboxamide |
| 67 | 14 | 3 | 2,6-dimethyl-4′-[(1-{[4-(propan-2- |
| | | | yl)phenyl]carbamoyl}-D-prolyl)amino][1,1′-biphenyl]-4- |
| | | | carboxylic acid |
| 68 | 38 | 3 | 4′-[(1-{[4-(propan-2-yl)phenyl]carbamoyl}-D- |
| | | | prolyl)amino]-3-(trifluoromethyl)[1,1′-biphenyl]-4- |
| | | | carboxylic acid |
| 69 | 47 | 2 | 3-fluoro-5-methyl-4′-[(1-{[4-(propan-2- |
| | | | yl)phenyl]carbamoyl}-D-prolyl)amino][1,1′-biphenyl]-4- |
| | | | carboxylic acid |
| 70 | 49 | 1 | 2-chloro-5-fluoro-4′-[(1-{[4-(propan-2- |
| | | | yl)phenyl]carbamoyl}-D-prolyl)amino][1,1′-biphenyl]-4- |
| | | | carboxylic acid |
| 71 | 2.5 | 3 | 2-(propan-2-yl)-4′-[(1-{[4-(propan-2- |
| | | | yl)phenyl]carbamoyl}-D-prolyl)amino][1,1′-biphenyl]-4- |
| | | | carboxylic acid |
| 72 | 69 | 1 | 4′-[(1-{[6-(trifluoromethyl)pyridin-3-yl]carbamoyl}-D- |
| | | | prolyl)amino][1,1′-biphenyl]-4-carboxylic acid |
| 73 | 12 | 4 | 4′-({1-[(3,4-dichlorophenyl)carbamoyl]-D- |
| | | | prolyl}amino)[1,1′-biphenyl]-4-carboxylic acid |
| 74 | 82 | 4 | 4′-({1-[(3-chlorophenyl)carbamoyl]-D- |
| | | | prolyl}amino)[1,1′-biphenyl]-4-carboxylic acid |
| 75 | 140 | 4 | 6-{2-fluoro-4-[(1-{[4-(propan-2-yl)phenyl]carbamoyl}- |
| | | | D-prolyl)amino]phenyl}pyridine-2-carboxylic acid |
| 76 | 9.9 | 7 | 2′-methyl-4′-[(1-{[4-(propan-2-yl)phenyl]carbamoyl}-D- |
| | | | prolyl)amino][1,1′-biphenyl]-4-carboxylic acid |
| 77 | 16 | 7 | 3-{6-[(1-{[4-(propan-2-yl)phenyl]carbamoyl}-D- |
| | | | prolyl)amino]pyridin-3-yl}benzoic acid |
| 78 | 145 | 4 | 3-{5-[(1-{[4-(propan-2-yl)phenyl]carbamoyl}-D- |
| | | | prolyl)amino]pyridin-2-yl}benzoic acid |
| 79 | 15 | 3 | 2-chloro-4′-[(1-{[4-(propan-2-yl)phenyl]carbamoyl}-D- |
| | | | prolyl)amino][1,1′-biphenyl]-4-carboxylic acid |
| 80 | 400 | 4 | 2,6-difluoro-4′-[(1-{[4-(propan-2-yl)phenyl]carbamoyl}- |
| | | | D-prolyl)amino][1,1′-biphenyl]-3-carboxylic acid |
| 81 | 210 | 4 | 5-fluoro-2-methoxy-4′-[(1-{[4-(propan-2- |
| | | | yl)phenyl]carbamoyl}-D-prolyl)amino][1,1′-biphenyl]-4- |
| | | | carboxylic acid |
| 82 | 27 | 3 | 2,5-difluoro-4′-[(1-{[4-(propan-2-yl)phenyl]carbamoyl}- |
| | | | D-prolyl)amino][1,1′-biphenyl]-4-carboxylic acid |
| 83 | 230 | 3 | 3-chloro-5-fluoro-4′-[(1-{[4-(propan-2- |
| | | | yl)phenyl]carbamoyl}-D-prolyl)amino][1,1′-biphenyl]-4- |
| | | | carboxylic acid |
| 84 | 19 | 5 | 5-fluoro-2-methyl-4′-[(1-{[4-(propan-2- |
| | | | yl)phenyl]carbamoyl}-D-prolyl)amino][1,1′-biphenyl]-4- |
| | | | carboxylic acid |
| 85 | 260 | 4 | 5-fluoro-4′-[(1-{[4-(propan-2-yl)phenyl]carbamoyl}-D- |
| | | | prolyl)amino][1,1′-biphenyl]-3-carboxylic acid |
| 86 | 7.5 | 4 | 2-fluoro-5-methyl-4′-[(1-{[4-(propan-2- |
| | | | yl)phenyl]carbamoyl}-D-prolyl)amino][1,1′-biphenyl]-4- |
| | | | carboxylic acid |
| 87 | 11 | 3 | 2-fluoro-3-methyl-4′-[(1-{[4-(propan-2- |
| | | | yl)phenyl]carbamoyl}-D-prolyl)amino][1,1′-biphenyl]-4- |
| | | | carboxylic acid |
| 88 | 430 | 5 | 2,4-difluoro-4′-[(1-{[4-(propan-2-yl)phenyl]carbamoyl}- |
| | | | D-prolyl)amino][1,1′-biphenyl]-3-carboxylic acid |
| 89 | 18 | 3 | 2-fluoro-5-methoxy-4′-[(1-{[4-(propan-2- |
| | | | yl)phenyl]carbamoyl}-D-prolyl)amino][1,1′-biphenyl]-4- |
| | | | carboxylic acid |
| 90 | 45 | 5 | 2-chloro-6-methyl-4′-[(1-{[4-(propan-2- |
| | | | yl)phenyl]carbamoyl}-D-prolyl)amino][1,1′-biphenyl]-4- |
| | | | carboxylic acid |
| 91 | 39 | 2 | (2R)-N²-(4′-carbamoyl[1,1′-biphenyl]-4-yl)-N¹-[4- |
| | | | (propan-2-yl)phenyl]pyrrolidine-1,2-dicarboxamide |
| 92 | 87 | 3 | 2-methoxy-6-methyl-4′-[(1-{[4-(propan-2- |
| | | | yl)phenyl]carbamoyl}-D-prolyl)amino][1,1′-biphenyl]-4- |
| | | | carboxylic acid |
| 93 | 79 | 2 | 4-methoxy-4′-[(1-{[4-(propan-2-yl)phenyl]carbamoyl}- |
| | | | D-prolyl)amino][1,1′-biphenyl]-3-carboxylic acid |
| 94 | 190 | 3 | 6-methyl-4′-[(1-{[4-(propan-2-yl)phenyl]carbamoyl}-D- |
| | | | prolyl)amino][1,1′-biphenyl]-3-carboxylic acid |
| 95 | 13 | 3 | 3-ethyl-4′-[(1-{[4-(propan-2-yl)phenyl]carbamoyl}-D- |
| | | | prolyl)amino][1,1′-biphenyl]-4-carboxylic acid |
| 96 | 180 | 4 | (2R)-N²-(3′-chloro-4′-hydroxy[1,1′-biphenyl]-4-yl)-N¹- |
| | | | [4-(propan-2-yl)phenyl]pyrrolidine-1,2-dicarboxamide |
| 97 | 230 | 3 | (2R)-N²-(4′-hydroxy-2′-methyl[1,1′-biphenyl]-4-yl)-N¹- |
| | | | [4-(propan-2-yl)phenyl]pyrrolidine-1,2-dicarboxamide |
| 98 | 30 | 3 | 5-chloro-2-fluoro-4′-[(1-{[4-(propan-2- |
| | | | yl)phenyl]carbamoyl}-D-prolyl)amino][1,1′-biphenyl]-4- |
| | | | carboxylic acid |
| 99 | 18 | 3 | (2R)-N²-(3′,5′-difluoro-4′-hydroxy[1,1′-biphenyl]-4-yl)- |
| | | | N¹-[4-(propan-2-yl)phenyl]pyrrolidine-1,2- |
| | | | dicarboxamide |
| 100 | 27 | 3 | (2R)-N²-(3′-cyano-4′-hydroxy[1,1′-biphenyl]-4-yl)-N¹- |
| | | | [4-(propan-2-yl)phenyl]pyrrolidine-1,2-dicarboxamide |
| 101 | 92 | 3 | 4′-[(1-{[2-(trifluoromethyl)pyrimidin-5-yl]carbamoyl}-D- |
| | | | prolyl)amino][1,1′-biphenyl]-4-carboxylic acid |
| 102 | 690 | 5 | 4′-[(1-{[6-methyl-5-(propan-2-yl)pyridin-2- |
| | | | yl]carbamoyl}-D-prolyl)amino][1,1′-biphenyl]-4- |
| | | | carboxylic acid |
| 103 | 29 | 3 | 4′-({1-[(2,4-dichlorophenyl)carbamoyl]-D- |
| | | | prolyl}amino)[1,1′-biphenyl]-4-carboxylic acid |
| 104 | 2.9 | 4 | 4′-{[(1R,2R,5S)-3-{[4-(propan-2-yl)phenyl]carbamoyl}- |
| | | | 3-azabicyclo[3.1.0]hexane-2-carbonyl]amino}[1,1′- |
| | | | biphenyl]-4-carboxylic acid |
| 105 | 8.8 | 7 | 4′-{[(1S,2R,5R)-3-{[4-(propan-2-yl)phenyl]carbamoyl}- |
| | | | 3-azabicyclo[3.1.0]hexane-2-carbonyl]amino}[1,1′- |
| | | | biphenyl]-4-carboxylic acid |
| 106 | 3.0 | 3 | 4′-[(1-{[3-methyl-4-(trifluoromethyl)phenyl]carbamoyl}- |
| | | | D-prolyl)amino][1,1′-biphenyl]-4-carboxylic acid |
| 107 | 720 | 6 | 4′-[(1-{[(4-chloro-3-cyanophenyl)methyl]carbamoyl}-D- |
| | | | prolyl)amino][1,1′-biphenyl]-4-carboxylic acid |
| 108 | 260 | 4 | 4′-{[1-({[2-(trifluoromethyl)pyrimidin-5- |
| | | | yl]methyl}carbamoyl)-D-prolyl]amino}[1,1′-biphenyl]-4- |
| | | | carboxylic acid |
| 109 | 5.1 | 3 | 4′-{[1-{{[4-(propan-2-yl)phenyl]methyl}carbamoyl)-D- |
| | | | prolyl]amino}[1,1′-biphenyl]-4-carboxylic acid |
| 110 | 3.7 | 5 | 4′-[(1-{[4-chloro-3-(trifluoromethyl)phenyl]carbamoyl}- |
| | | | D-prolyl)amino][1,1′-biphenyl]-4-carboxylic acid |
| 111 | 11 | 4 | 4′-[(1-{[4-(butan-2-yl)phenyl]carbamoyl}-D- |
| | | | prolyl)amino][1,1′-biphenyl]-4-carboxylic acid |
| 112 | 840 | 4 | 4′-[(1-{[2-(propan-2-yl)pyrimidin-5-yl]carbamoyl}-D- |
| | | | prolyl)amino][1,1′-biphenyl]-4-carboxylic acid |
| 113 | 21 | 4 | 4′-[(1-{[(3,5-dichlorophenyl)methyl]carbamoyl}-D- |
| | | | prolyl)amino][1,1′-biphenyl]-4-carboxylic acid |
| 114 | 40 | 5 | 4′-({1-[(4-cyclohexylphenyl)carbamoyl]-D- |
| | | | prolyl}amino)[1,1′-biphenyl]-4-carboxylic acid |
| 115 | 4.8 | 4 | 4′-({1-[(4-cyclopentylphenyl)carbamoyl]-D- |
| | | | prolyl}amino)[1,1′-biphenyl]-4-carboxylic acid |
| 116 | 12 | 3 | 4′-[(1-{[(3,4-dichlorophenyl)methyl]carbamoyl}-D- |
| | | | prolyl)amino][1,1′-biphenyl]-4-carboxylic acid |
| 117 | 26 | 3 | 4′-[(1-{[2-fluoro-4-(propan-2-yl)phenyl]carbamoyl}-D- |
| | | | prolyl)amino][1,1′-biphenyl]-4-carboxylic acid |
| 118 | 15 | 3 | 4′-({1-[(4-ethylphenyl)carbamoyl]-D-prolyl}amino)[1,1′- |
| | | | biphenyl]-4-carboxylic acid |
| 119 | 24 | 3 | 4′-({1-[(4-tert-butylphenyl)carbamoyl]-D- |
| | | | prolyl}amino)[1,1′-biphenyl]-4-carboxylic acid |
| 120 | 26 | 4 | 6-methoxy-5-{4-[(1-{[4-(propan-2- |
| | | | yl)phenyl]carbamoyl}-D-prolyl)amino]phenyl}pyridine- |
| | | | 2-carboxylic acid |
| 121 | 27 | 4 | 4-{5-[(1-{[4-(propan-2-yl)phenyl]carbamoyl}-D- |
| | | | prolyl)amino]pyrazin-2-yl}benzoic acid |
| 122 | 99 | 4 | 5-[4-({1-[(4-cyclopropylphenyl)carbamoyl]-D- |
| | | | prolyl}amino)phenyl]pyridine-2-carboxylic acid |
| 123 | 11 | 4 | 4-(6-{[(1R,2R,5S)-3-{[4-(propan-2- |
| | | | yl)phenyl]carbamoyl}-3-azabicyclo[3.1.0]hexane-2- |
| | | | carbonyl]amino}pyridin-3-yl)benzoic acid |
| 124 | 74 | 3 | 4′-[(1-{[(3-chloro-4-fluorophenyl)methyl]carbamoyl}-D- |
| | | | prolyl)amino][1,1′-biphenyl]-4-carboxylic acid |
| 125 | 19 | 6 | 4′-[(1-{[(4-tert-butylphenyl)methyl]carbamoyl}-D- |
| | | | prolyl)amino][1,1′-biphenyl]-4-carboxylic acid |
| 126 | 6.6 | 4 | 4′-{[1-({[3-fluoro-4- |
| | | | (trifluoromethyl)phenyl]methyl}carbamoyl)-D- |
| | | | prolyl]amino}[1,1′-biphenyl]-4-carboxylic acid |
| 127 | 69 | 3 | 4′-{[1-({[2-fluoro-4-(propan-2- |
| | | | yl)phenyl]methyl}carbamoyl)-D-prolyl]amino}[1,1′- |
| | | | biphenyl]-4-carboxylic acid |
| 128 | 9.3 | 3 | 4′-[(1-{[3,5-difluoro-4- |
| | | | (trifluoromethyl)phenyl]carbamoyl}-D- |
| | | | prolyl)amino][1,1′-biphenyl]-4-carboxylic acid |
| 129 | 14 | 4 | 4′-[(1-{[(3-fluoro-4-methylphenyl)methyl]carbamoyl}-D- |
| | | | prolyl)amino][1,1′-biphenyl]-4-carboxylic acid |
| 130 | 11 | 4 | 4′-{[1-({[3-chloro-4- |
| | | | (trifluoromethyl)phenyl]methyl}carbamoyl)-D- |
| | | | prolyl]amino}[1,1′-biphenyl]-4-carboxylic acid |
| 131 | 140 | 3 | 4′-{[1-({[2-fluoro-4- |
| | | | (trifluoromethyl)phenyl]methyl}carbamoyl)-D- |
| | | | prolyl]amino}[1,1′-biphenyl]-4-carboxylic acid |
| 132 | 77 | 4 | 4′-[(1-{[(4-chloro-2-fluorophenyl)methyl]carbamoyl}-D- |
| | | | prolyl)amino][1,1′-biphenyl]-4-carboxylic acid |
| 133 | 180 | 4 | 4′-[(1-{[(4-chloro-2,6- |
| | | | difluorophenyl)methyl]carbamoyl}-D- |
| | | | prolyl)amino][1,1′-biphenyl]-4-carboxylic acid |
| 134 | 140 | 4 | 4′-[(1-{[2-methoxy-4- |
| | | | (trifluoromethyl)phenyl]carbamoyl}-D- |
| | | | prolyl)amino][1,1′-biphenyl]-4-carboxylic acid |
| 135 | 52 | 3 | 4′-[(1-{[2-chloro-4-(propan-2-yl)phenyl]carbamoyl}-D- |
| | | | prolyl)amino][1,1′-biphenyl]-4-carboxylic acid |
| 136 | 7.8 | 3 | 4′-[(1-{[(4-cyclopropylphenyl)methyl]carbamoyl}-D- |
| | | | prolyl)amino][1,1′-biphenyl]-4-carboxylic acid |
| 137 | 9.8 | 3 | 4′-{[1-{{[4-(2-methylpropyl)phenyl]methyl}carbamoyl)- |
| | | | D-prolyl]amino}[1,1′-biphenyl]-4-carboxylic acid |
| 138 | 42 | 3 | 4′-[(1-{[(4-chloro-2,5- |
| | | | difluorophenyl)methyl]carbamoyl}-D- |
| | | | prolyl)amino][1,1′-biphenyl]-4-carboxylic acid |
| 139 | 93 | 3 | 4′-{[1-({[2-chloro-4- |
| | | | (trifluoromethyl)phenyl]methyl}carbamoyl)-D- |
| | | | prolyl]amino}[1,1′-biphenyl]-4-carboxylic acid |
| 140 | 67 | 3 | 4′-({1-[(4-cyclopropyl-2-fluorophenyl)carbamoyl]-D- |
| | | | prolyl}amino)[1,1′-biphenyl]-4-carboxylic acid |
| 141 | 97 | 3 | 4′-{[1-({[4-(difluoromethyl)phenyl]methyl}carbamoyl)- |
| | | | D-prolyl]amino}[1,1′-biphenyl]-4-carboxylic acid |
| 142 | 130 | 3 | 4′-({1-[(4-cyclopropyl-2-methylphenyl)carbamoyl]-D- |
| | | | prolyl}amino)[1,1′-biphenyl]-4-carboxylic acid |
| 143 | 35 | 1 | 4′-[(1-{[3-methoxy-4- |
| | | | (trifluoromethyl)phenyl]carbamoyl}-D- |
| | | | prolyl)amino][1,1′-biphenyl]-4-carboxylic acid |
| 144 | 6.1 | 7 | 4′-({1-[(4-cyclopropyl-3-methylphenyl)carbamoyl]-D- |
| | | | prolyl}amino)[1,1′-biphenyl]-4-carboxylic acid |
| 145 | 70 | 3 | 4′-[(1-{[5-methyl-6-(propan-2-yl)pyridin-3- |
| | | | yl]carbamoyl}-D-prolyl)amino][1,1′-biphenyl]-4- |
| | | | carboxylic acid, trifluoroacetate salt |
| 146 | 9.7 | 4 | 4′-[(1-{[4-methyl-5-(propan-2-yl)pyridin-2- |
| | | | yl]carbamoyl}-D-prolyl)amino][1,1′-biphenyl]-4- |
| | | | carboxylic acid, trifluoroacetate salt |
| 147 | 13 | 3 | 4′-[(1-{[4-(1,1, 1-trifluoropropan-2- |
| | | | yl)phenyl]carbamoyl}-D-prolyl)amino][1,1′-biphenyl]-4- |
| | | | carboxylic acid |
| 148 | 240 | 3 | (2R)-N²-(3′-carbamoyl[1,1′-biphenyl]-4-yl)-N¹-[4- |
| | | | (propan-2-yl)phenyl]pyrrolidine-1,2-dicarboxamide |
| 149 | 21 | 5 | 4′-({1-[(4-cyclopropyl-3-fluorophenyl)carbamoyl]-D- |
| | | | prolyl}amino)[1,1′-biphenyl]-4-carboxylic acid |
| 150 | 86 | 3 | 4-{3-methyl-5-[(1-{[4-(propan-2-yl)phenyl]carbamoyl}- |
| | | | D-prolyl)amino]pyridin-2-yl}benzoic acid |
| 151 | 110 | 3 | 3-methoxy-4-{6-[(1-{[4-(propan-2- |
| | | | yl)phenyl]carbamoyl}-D-prolyl)amino]pyridin-3- |
| | | | yl}benzoic acid |
| 152 | 44 | 3 | 6-[(1-{[3-fluoro-4-(propan-2-yl)phenyl]carbamoyl}-D- |
| | | | prolyl)amino]naphthalene-2-carboxylic acid |
| 153 | 11 | 5 | 4-(6-{[1-({[4- |
| | | | (trifluoromethyl)phenyl]methyl}carbamoyl)-D- |
| | | | prolyl]amino}pyridin-3-yl)benzoic acid |
| 154 | 58 | 3 | 4′-{[(1R,2R,5S)-3-{[4-(propan-2-yl)phenyl]carbamoyl}- |
| | | | 3-azabicyclo[3.1.0]hexane-2-carbonyl]amino}[1,1′- |
| | | | biphenyl]-3-carboxylic acid |
| 155 | 22 | 4 | 4′-[(1-{[4-(2,2,2-trifluoroethyl)phenyl]carbamoyl}-D- |
| | | | prolyl)amino][1,1′-biphenyl]-4-carboxylic acid |
| 156 | 45 | 4 | 3-methoxy-4-{6-[(1-{[3-methyl-4-(propan-2- |
| | | | yl)phenyl]carbamoyl}-D-prolyl)amino]pyridin-3- |
| | | | yl}benzoic acid |
| 157 | 10 | 7 | 4′-{[1-({[4-(trifluoromethyl)phenyl]methyl}carbamoyl)- |
| | | | D-prolyl]amino}[1,1′-biphenyl]-4-carboxylic acid |
| 158 | 7.7 | 6 | 3-methoxy-4′-[(1-{[3-methyl-4- |
| | | | (trifluoromethyl)phenyl]carbamoyl}-D- |
| | | | prolyl)amino][1,1′-biphenyl]-4-carboxylic acid |
| 159 | 110 | 4 | 4′-{[(1S,2R,5R)-3-{[4-(propan-2-yl)phenyl]carbamoyl}- |
| | | | 3-azabicyclo[3.1.0]hexane-2-carbonyl]amino}[1,1′- |
| | | | biphenyl]-3-carboxylic acid |
| 160 | 75 | 3 | 4-[5-({1-[(4-cyclobutylphenyl)carbamoyl]-D- |
| | | | prolyl}amino)-3-methylpyridin-2-yl]benzoic acid |
| 161 | 77 | 3 | 6-[(1-{[4-(trifluoromethyl)phenyl]carbamoyl}-D- |
| | | | prolyl)amino]naphthalene-2-carboxylic acid |
| 162 | 44 | 3 | 6-[(1-{[3-methyl-4-(propan-2-yl)phenyl]carbamoyl}-D- |
| | | | prolyl)amino]naphthalene-2-carboxylic acid |
| 163 | 19 | 5 | 4-{5-[(1-{[3-methyl-4- |
| | | | (trifluoromethyl)phenyl]carbamoyl}-D- |
| | | | prolyl)amino]pyridin-2-yl}benzoic acid |
| 164 | 22 | 4 | 4′-{[1-({(1S)-1-[4- |
| | | | (trifluoromethyl)phenyl]ethyl}carbamoyl)-D- |
| | | | prolyl]amino}[1,1′-biphenyl]-4-carboxylic acid |
| 165 | 4.6 | 3 | 4′-{[(3R)-3-ethyl-1-{[4-(propan-2-yl)phenyl]carbamoyl}- |
| | | | L-prolyl]amino}[1,1′-biphenyl]-4-carboxylic acid |
| | | | or |
| | | | 4′-{[(3S)-3-ethyl-1-{[4-(propan-2-yl)phenyl]carbamoyl}- |
| | | | D-prolyl]amino}[1,1′-biphenyl]-4-carboxylic acid |
| | | | [From DIAST-2 of precursor (see footnote 27 in Table |
| | | | 2] |
| 166 | 5.7 | 3 | 4′-{[(3R)-3-ethyl-1-{[4-(propan-2-yl)phenyl]carbamoyl}- |
| | | | D-prolyl]amino}[1,1′-biphenyl]-4-carboxylic acid |
| | | | or |
| | | | 4′-{[(3S)-3-ethyl-1-{[4-(propan-2-yl)phenyl]carbamoyl}- |
| | | | L-prolyl]amino}[1,1′-biphenyl]-4-carboxylic acid |
| | | | [From DIAST-2 of precursor (see footnote 27 in Table |
| | | | 2] |
| 167 | 7.2 | 7 | 6-methyl-5-{4-[(1-{[3-methyl-4- |
| | | | (trifluoromethyl)phenyl]carbamoyl}-D- |
| | | | prolyl)amino]phenyl}pyridine-2-carboxylic acid |
| 168 | 15 | 5 | 6-methyl-5-{4-[(1-{[3-methyl-4-(propan-2- |
| | | | yl)phenyl]carbamoyl}-D-prolyl)amino]phenyl}pyridine- |
| | | | 2-carboxylic acid |
| 169 | 49 | 3 | 6-methyl-5-{4-[(1-{[4- |
| | | | (trifluoromethyl)phenyl]carbamoyl}-D- |
| | | | prolyl)amino]phenyl}pyridine-2-carboxylic acid |
| 170 | 40 | 3 | 3-methoxy-4′-[(1-{[4- |
| | | | (trifluoromethyl)phenyl]carbamoyl}-D- |
| | | | prolyl)amino][1,1′-biphenyl]-4-carboxylic acid |
| 171 | 16 | 4 | 3-methyl-4-{6-[(1-{[4-(propan-2-yl)phenyl]carbamoyl}- |
| | | | D-prolyl)amino]pyridin-3-yl}benzoic acid |
| 172 | 31 | 4 | 3-methyl-4-{6-[(1-{[4- |
| | | | (trifluoromethyl)phenyl]carbamoyl}-D- |
| | | | prolyl)amino]pyridin-3-yl}benzoic acid |
| 173 | 4.4 | 3 | 3-methyl-4-{6-[(1-{[3-methyl-4-(propan-2- |
| | | | yl)phenyl]carbamoyl}-D-prolyl)amino]pyridin-3- |
| | | | yl}benzoic acid |
| 174 | 49 | 5 | 5-[4-({1-[(4-cyclopropyl-3-methylphenyl)carbamoyl]-D- |
| | | | prolyl}amino)phenyl]pyridine-2-carboxylic acid |
| 175 | 8.3 | 4 | 3-{6-[(1-{[3-methyl-4- |
| | | | (trifluoromethyl)phenyl]carbamoyl}-D- |
| | | | prolyl)amino]pyridin-3-yl}benzoic acid |
| 176 | 11 | 3 | 4-{6-[(1-{[3-fluoro-4-(propan-2-yl)phenyl]carbamoyl}- |
| | | | D-prolyl)amino]pyridin-3-yl}-3-methylbenzoic acid |
| 177 | 8.2 | 5 | 4-{6-[(1-{[3-methyl-4- |
| | | | (trifluoromethyl)phenyl]carbamoyl}-D- |
| | | | prolyl)amino]pyridin-3-yl}benzoic acid |
| 178 | 11 | 4 | 4-(6-{[1-({4-[1- |
| | | | (trifluoromethyl)cyclopropyl]phenyl}carbamoyl)-D- |
| | | | prolyl]amino}pyridin-3-yl)benzoic acid |
| 179 | 3.7 | 3 | 4-[6-({1-[(4-cyclopropyl-3-methylphenyl)carbamoyl]-D- |
| | | | prolyl}amino)pyridin-3-yl]benzoic acid |
| 180 | 53 | 4 | 6-{4-[(1-{[3-methyl-4-(propan-2-yl)phenyl]carbamoyl}- |
| | | | D-prolyl)amino]phenyl}pyridine-3-carboxylic acid |
| 181 | 24 | 4 | 3-{6-[(1-{[4-(trifluoromethyl)phenyl]carbamoyl}-D- |
| | | | prolyl)amino]pyridin-3-yl}benzoic acid |
| 182 | 23 | 4 | 6-{4-[(1-{[3-fluoro-4-(propan-2-yl)phenyl]carbamoyl}- |
| | | | D-prolyl)amino]phenyl}pyridine-3-carboxylic acid |
| 183 | 9.3 | 5 | 3-{6-[(1-{[3-methyl-4-(propan-2-yl)phenyl]carbamoyl}- |
| | | | D-prolyl)amino]pyridin-3-yl}benzoic acid |
| 184 | 43 | 3 | 3-{6-[(1-{[4-methyl-5-(propan-2-yl)pyridin-2- |
| | | | yl]carbamoyl}-D-prolyl)amino]pyridin-3-yl}benzoic acid |
| 185 | 4.2 | 4 | 2-methyl-4-{6-[(1-{[3-methyl-4-(propan-2- |
| | | | yl)phenyl]carbamoyl}-D-prolyl)amino]pyridin-3- |
| | | | yl}benzoic acid |
| 186 | 36 | 3 | 2-fluoro-4-{6-[(1-{[3-methyl-4- |
| | | | (trifluoromethyl)phenyl]carbamoyl}-D- |
| | | | prolyl)amino]pyridin-3-yl}benzoic acid |
| 187 | 51 | 4 | 2-fluoro-4-{6-[(1-{[3-methyl-4-(propan-2- |
| | | | yl)phenyl]carbamoyl}-D-prolyl)amino]pyridin-3- |
| | | | yl}benzoic acid |
| 188 | 32 | 3 | 3-{6-[(1-{[4-chloro-3- |
| | | | (trifluoromethyl)phenyl]carbamoyl}-D- |
| | | | prolyl)amino]pyridin-3-yl}benzoic acid |
| 189 | 42 | 3 | 2-fluoro-4-(6-{[1-({[4-(propan-2- |
| | | | yl)phenyl]methyl}carbamoyl)-D-prolyl]amino}pyridin-3- |
| | | | yl)benzoic acid |
| 190 | 9.9 | 3 | 4-(6-{[1-({[3-fluoro-4- |
| | | | (trifluoromethyl)phenyl]methyl}carbamoyl)-D- |
| | | | prolyl]amino}pyridin-3-yl)-2-methylbenzoic acid |
| 191 | 14 | 3 | 2-methyl-4-(6-{[1-{{[4-(propan-2- |
| | | | yl)phenyl]methyl}carbamoyl)-D-prolyl]amino}pyridin-3- |
| | | | yl)benzoic acid |
| 192 | 12 | 3 | 2-methyl-4-(6-{[1-({[4- |
| | | | (trifluoromethyl)phenyl]methyl}carbamoyl)-D- |
| | | | prolyl]amino}pyridin-3-yl)benzoic acid |
| 193 | 17 | 3 | 4-{3-fluoro-5-[(1-{[4- |
| | | | (trifluoromethyl)phenyl]carbamoyl}-D- |
| | | | prolyl)amino]pyridin-2-yl}benzoic acid |
| 194 | 14 | 4 | 4-{5-[(1-{[3-fluoro-4-(propan-2-yl)phenyl]carbamoyl}- |
| | | | D-prolyl)amino]pyridin-2-yl}benzoic acid |
| 195 | 35 | 6 | 5-(4-{[1-({[4- |
| | | | (trifluoromethyl)phenyl]methyl}carbamoyl)-D- |
| | | | prolyl]amino}phenyl)pyridine-2-carboxylic acid |
| 196 | 49 | 3 | 5-{4-[(1-{[4-(trifluoromethyl)phenyl]carbamoyl}-D- |
| | | | prolyl)amino]phenyl}pyridine-2-carboxylic acid |
| 197 | 29 | 3 | 5-(4-{[1-({4-[1- |
| | | | (trifluoromethyl)cyclopropyl]phenyl}carbamoyl)-D- |
| | | | prolyl]amino}phenyl)pyridine-2-carboxylic acid |
| 198 | 69 | 3 | 4-{5-[(1-{[4-methyl-5-(propan-2-yl)pyridin-2- |
| | | | yl]carbamoyl}-D-prolyl)amino]pyridin-2-yl}benzoic acid |
| 199 | 71 | 2 | 2-fluoro-4-{6-[(1-{[4-(propan-2-yl)phenyl]carbamoyl}- |
| | | | D-prolyl)amino]pyridin-3-yl}benzoic acid |
| 200 | 9.9 | 4 | 2-methyl-4-{6-[(1-{[4-(propan-2-yl)phenyl]carbamoyl}- |
| | | | D-prolyl)amino]pyridin-3-yl}benzoic acid |
| 201 | 91 | 3 | ammonium 6′-[(1-{[3-methyl-4-(propan-2- |
| | | | yl)phenyl]carbamoyl}-D-prolyl)amino][3,3′-bipyridine]- |
| | | | 6-carboxylate |
| 202 | 22 | 3 | 3-{6-[(1-{[4-methyl-5-(trifluoromethyl)pyridin-2- |
| | | | yl]carbamoyl}-D-prolyl)amino]pyridin-3-yl}benzoic acid |
| 203 | 8.4 | 4 | 4′-[(1-{[4-methyl-5-(trifluoromethyl)pyridin-2- |
| | | | yl]carbamoyl}-D-prolyl)amino][1,1′-biphenyl]-4- |
| | | | carboxylic acid |
| 204 | 14 | 4 | 3-fluoro-4′-[(1-{[4-(trifluoromethyl)phenyl]carbamoyl}- |
| | | | D-prolyl)amino][1,1′-biphenyl]-4-carboxylic acid |
| 205 | 14 | 3 | 6-methyl-5-(4-{[1-({[4-(propan-2- |
| | | | yl)phenyl]methyl}carbamoyl)-D- |
| | | | prolyl]amino}phenyl)pyridine-2-carboxylic acid |
| 206 | 14 | 4 | 6-methyl-5-(4-{[1-{{[4- |
| | | | (trifluoromethyl)phenyl]methyl}carbamoyl)-D- |
| | | | prolyl]amino}phenyl)pyridine-2-carboxylic acid |
| 207 | 1.3 | 5 | 4-{6-[(1-{[3-fluoro-4-(propan-2-yl)phenyl]carbamoyl}- |
| | | | D-prolyl)amino]pyridin-3-yl}benzoic acid |
| 208 | 4.9 | 5 | 5-{4-[(1-{[3-fluoro-4-(propan-2-yl)phenyl]carbamoyl}- |
| | | | D-prolyl)amino]phenyl}pyridine-2-carboxylic acid |
| 209 | 7.4 | 4 | 3-methyl-4′-[(1-{[4-(trifluoromethyl)phenyl]carbamoyl}- |
| | | | D-prolyl)amino][1,1′-biphenyl]-4-carboxylic acid |
| 210 | 9.3 | 3 | 4-[6-({1-[(4-cyclobutylphenyl)carbamoyl]-D- |
| | | | prolyl}amino)pyridin-3-yl]benzoic acid |
| 211 | 7.0 | 4 | 5-[4-({1-[(4-cyclobutylphenyl)carbamoyl]-D- |
| | | | prolyl}amino)phenyl]pyridine-2-carboxylic acid |
| 212 | 18 | 3 | 5-{4-[(1-{[(3-fluoro-4-methylphenyl)methyl]carbamoyl}- |
| | | | D-prolyl)amino]phenyl}-6-methylpyridine-2-carboxylic |
| | | | acid |
| 213 | 90 | 3 | 5-{4-[(1-{[5-fluoro-6-(propan-2-yl)pyridin-3- |
| | | | yl]carbamoyl}-D-prolyl)amino]phenyl}-6- |
| | | | methylpyridine-2-carboxylic acid |
| 214 | 6.7 | 4 | 3-fluoro-4′-[(1-{[3-fluoro-4-(propan-2- |
| | | | yl)phenyl]carbamoyl}-D-prolyl)amino][1,1′-biphenyl]-4- |
| | | | carboxylic acid |
| 215 | 15 | 3 | 4-{5-[(1-{[3-fluoro-4- |
| | | | (trifluoromethyl)phenyl]carbamoyl}-D- |
| | | | prolyl)amino]pyridin-2-yl}benzoic acid |
| 216 | 65 | 3 | 6-methyl-5-{4-[(1-{[4-methyl-5-(propan-2-yl)pyridin-2- |
| | | | yl]carbamoyl}-D-prolyl)amino]phenyl}pyridine-2- |
| | | | carboxylic acid, trifluoroacetate salt |
| 217 | 140 | 2 | 2-[(1-{[4-(trifluoromethyl)phenyl]carbamoyl}-D- |
| | | | prolyl)amino]-5,6,7,8-tetrahydroquinoline-6-carboxylic |
| | | | acid, DIAST-1 (see footnote 43 in Table 2) |
| 218 | 33 | 3 | 2-[(1-{[4-(trifluoromethyl)phenyl]carbamoyl}-D- |
| | | | prolyl)amino]-5,6,7,8-tetrahydroquinoline-6-carboxylic |
| | | | acid, DIAST-2 (see footnote 43 in Table 2) |
| 219 | 2.9 | 3 | 4′-[(1-{[3-fluoro-4-(propan-2-yl)phenyl]carbamoyl}-D- |
| | | | prolyl)amino]-2′-methyl[1,1′-biphenyl]-4-carboxylic |
| | | | acid |
| 220 | 47 | 3 | 2-[(1-{[3-fluoro-4-(trifluoromethyl)phenyl]carbamoyl}- |
| | | | D-prolyl)amino]-5,6,7,8-tetrahydroquinoline-6- |
| | | | carboxylic acid, DIAST-1 (see footnote 46 in Table 2) |
| 221 | 11 | 4 | 2-[(1-{[3-fluoro-4-(trifluoromethyl)phenyl]carbamoyl}- |
| | | | D-prolyl)amino]-5,6,7,8-tetrahydroquinoline-6- |
| | | | carboxylic acid, DIAST-2 |
| | | | (see footnote 46 in Table 2) |
| 222 | 100 | 3 | 4-{5-[(1-{[4-methyl-5-(trifluoromethyl)pyridin-2- |
| | | | yl]carbamoyl}-D-prolyl)amino]pyridin-2-yl}benzoic acid |
| 223 | 51 | 3 | 3-(6-{[1-({[3-fluoro-4- |
| | | | (trifluoromethyl)phenyl]methyl}carbamoyl)-D- |
| | | | prolyl]amino}pyridin-3-yl)benzoic acid |
| 224 | 79 | 5 | 3-(6-{[1-{{[4-(propan-2-yl)phenyl]methyl}carbamoyl)- |
| | | | D-prolyl]amino}pyridin-3-yl) benzoic acid |
| 225 | 51 | 3 | 3-(6-{[1-({[4- |
| | | | (trifluoromethyl)phenyl]methyl}carbamoyl)-D- |
| | | | prolyl]amino}pyridin-3-yl)benzoic acid |
| 226 | 81 | 3 | 2-[(1-{[3-methyl-4-(trifluoromethyl)phenyl]carbamoyl}- |
| | | | D-prolyl)amino]-5,6,7,8-tetrahydroquinoline-6- |
| | | | carboxylic acid, DIAST-1 (see footnote 48 in Table 2) |
| 227 | 53 | 3 | 2-[(1-{[3-methyl-4-(trifluoromethyl)phenyl]carbamoyl}- |
| | | | D-prolyl)amino]-5,6,7,8-tetrahydroquinoline-6- |
| | | | carboxylic acid, DIAST-2 (see footnote 48 in Table 2) |
| 228 | 36 | 3 | 2-[(1-{[3-fluoro-4-(propan-2-yl)phenyl]carbamoyl}-D- |
| | | | prolyl)amino]-5,6,7,8-tetrahydroquinoline-6-carboxylic |
| | | | acid, trifluoroacetate salt, DIAST-1 (see footnote 49 in |
| | | | Table 2) |
| 229 | 11 | 3 | 2-[(1-{[3-fluoro-4-(propan-2-yl)phenyl]carbamoyl}-D- |
| | | | prolyl)amino]-5,6,7,8-tetrahydroquinoline-6-carboxylic |
| | | | acid, trifluoroacetate salt, DIAST-2 (see footnote 49 in |
| | | | Table 2) |

¹Values represent the geometric mean
²Values include the overall geometric mean of multiple forms (including salts) tested

Throughout this application, various publications are referenced. The disclosures of these publications in their entireties are hereby incorporated by reference into this application for all purposes.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

1. A compound of Formula I: 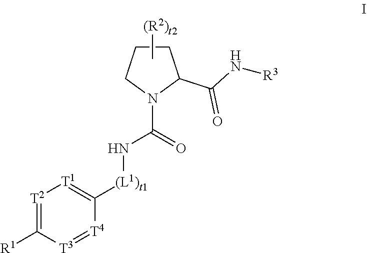
or a pharmaceutically acceptable salt thereof, wherein:
R¹ is H, halogen, —CN, C₁₋₈ alkyl, C₂₋₈ alkenyl, (C₃₋₆ cycloalkyl)-C₁₋₄ alkyl-, or C₃₋₆ cycloalkyl, wherein each of the C₁₋₈ alkyl, C₂₋₈ alkenyl, (C₃₋₆ cycloalkyl)-C₁₋₄ alkyl-, or C₃₋₆ cycloalkyl is optionally substituted with 1, 2, 3, 4, 5, or 6 substituents each independently selected from halogen, —OH, —CN, C₁₋₄ alkyl, C₁₋₄ haloalkyl, C₁₋₄ alkoxy, and C₁₋₄ haloalkoxy;
each R² is independently halogen, —OH, C₁₋₄ alkyl, C₁₋₄ haloalkyl, C₁₋₄ alkoxy, C₁₋₄ haloalkoxy, C₃₋₄ cycloalkyl, or (C₃₋₄ cycloalkyl)-C₁₋₄ alkyl-, wherein each of the C₁₋₄ alkyl, C₁₋₄ haloalkyl, C₁₋₄ alkoxy, C₁₋₄ haloalkoxy, C₃₋₄ cycloalkyl, or (C₃₋₄ cycloalkyl)-C₁₋₄ alkyl- is optionally substituted with 1, 2, or 3 substituents each independently selected from halogen, —OH, C₁₋₄ alkyl, C₁₋₄ haloalkyl, C₁₋₄ alkoxy, and C₁₋₄ haloalkoxy;
or two R², when attached to a same ring carbon atom of the proline ring in Formula I, together with the ring carbon atom to which they are attached, optionally form C₃₋₆ cycloalkyl or a 4- to 7-membered heterocycloalkyl, each of which is optionally substituted with 1, 2, 3, or 4 substituents each independently selected from halogen, —OH, C₁₋₄ alkyl, C₁₋₄ haloalkyl, C₁₋₄ alkoxy, and C₁₋₄ haloalkoxy;
or two R², when attached to two adjacent ring carbon atoms of the proline ring in Formula I, together with the two ring carbon atoms to which they are attached, optionally form C₃₋₆ cycloalkyl or a 4- to 7-membered heterocycloalkyl, each of which is optionally substituted with 1, 2, 3, or 4 substituents each independently selected from halogen, —OH, C₁₋₄ alkyl, C₁₋₄ haloalkyl, C₁₋₄ alkoxy, and C₁₋₄ haloalkoxy;
R³ is R^{3a}, R^{3b}, R^{3c}, or R^{3d}: 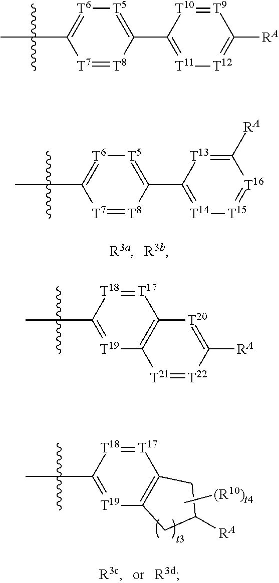
each of T¹, T², T³, and T⁴ is independently CR⁴ or N, provided that only 0, 1, or 2 of T¹, T², T³, and T⁴ can be N;
each R⁴ is independently H, halogen, —CN, C₃₋₆ cycloalkyl, (C₃₋₆ cycloalkyl)-C₁₋₂ alkyl-, C₁₋₄ alkyl, C₁₋₄ cyanoalkyl, C₁₋₄ haloalkyl, C₁₋₄ alkoxy, or C₁₋₄ haloalkoxy;
each of T⁵, T⁶, T⁷, and T⁸ is independently CR⁵ or N, provided that only 0, 1, or 2 of T⁵, T⁶, T⁷, and T⁸ can be N;
each R⁵ is independently H, halogen, —CN, C₃₋₆ cycloalkyl, (C₃₋₆ cycloalkyl)-C₁₋₂ alkyl-, C₁₋₄ alkyl, C₁₋₄ cyanoalkyl, C₁₋₄ haloalkyl, C₁₋₄ alkoxy, or C₁₋₄ haloalkoxy;
each of T⁹, T¹⁰, T¹¹, and T¹² is independently CR⁶ or N, provided that only 0, 1, or 2 of T⁹, T¹⁰, T¹¹, and T¹² can be N;
each R⁶ is independently H, halogen, —CN, C₃₋₆ cycloalkyl, (C₃₋₆ cycloalkyl)-C₁₋₂ alkyl-, C₁₋₄ alkyl, C₁₋₄ cyanoalkyl, C₁₋₄ haloalkyl, C₁₋₄ alkoxy, or C₁₋₄ haloalkoxy;
each of T¹³, T¹⁴, T¹⁵, and T¹⁶ is independently CR⁷ or N, provided that only 0, 1, or 2 of T¹³, T¹⁴, T¹⁵, and T¹⁶ can be N;
each R⁷ is independently H, halogen, —CN, C₃₋₆ cycloalkyl, (C₃₋₆ cycloalkyl)-C₁₋₂ alkyl-, C₁₋₄ alkyl, C₁₋₄ cyanoalkyl, C₁₋₄ haloalkyl, C₁₋₄ alkoxy, or C₁₋₄ haloalkoxy;
each of T¹⁷, T¹⁸, and T¹⁹ is independently CR⁸ or N, provided that only 0, 1, or 2 of T¹⁷, T¹⁸, and T¹⁹ can be N;
each R⁸ is independently H, halogen, —CN, C₃₋₆ cycloalkyl, (C₃₋₆ cycloalkyl)-C₁₋₂ alkyl-, C₁₋₄ alkyl, C₁₋₄ cyanoalkyl, C₁₋₄ haloalkyl, C₁₋₄ alkoxy, or C₁₋₄ haloalkoxy;
each of T²⁰, T²¹, and T²² is independently CR⁹ or N, provided that only 0, 1, or 2 of T²⁰, T²¹, and T²² can be N;
each R⁹ is independently H, halogen, —CN, C₃₋₆ cycloalkyl, (C₃₋₆ cycloalkyl)-C₁₋₂ alkyl-, C₁₋₄ alkyl, C₁₋₄ cyanoalkyl, C₁₋₄ haloalkyl, C₁₋₄ alkoxy, or C₁₋₄ haloalkoxy;
each R¹⁰ is independently halogen, —OH, C₁₋₄ alkyl, C₁₋₄ haloalkyl, C₁₋₄ alkoxy, C₁₋₄ haloalkoxy, C₃₋₄ cycloalkyl, or (C₃₋₄ cycloalkyl)-C₁₋₄ alkyl-, wherein each of the C₁₋₄ alkyl, C₁₋₄ haloalkyl, C₁₋₄ alkoxy, C₁₋₄ haloalkoxy, C₃₋₄ cycloalkyl, or (C₃₋₄ cycloalkyl)-C₁₋₄ alkyl- is optionally substituted with 1, 2, or 3 substituents each independently selected from halogen, —OH, C₁₋₄ alkyl, C₁₋₄ haloalkyl, C₁₋₄ alkoxy, and C₁₋₄ haloalkoxy;
R^{A} is —C(═O)—OH, 1H-tetrazol-5-yl, OH, —C(═O)—N(R¹¹)(R¹²), —C(═O)—OR¹³, 3-hydroxyisoxazol-5-yl, or —S(═O)₂NHCF₃;
each of R¹¹ and R¹² is independently H, C₁₋₆ alkyl, C₃₋₆ cycloalkyl, (C₃₋₆ cycloalkyl)-C₁₋₄ alkyl-, phenyl, or phenyl-C₁₋₄ alkyl-, wherein each of the C₁₋₆ alkyl, C₃₋₆ cycloalkyl, (C₃₋₆ cycloalkyl)-C₁₋₄ alkyl-, phenyl, or phenyl-C₁₋₄ alkyl- is optionally substituted with 1, 2, 3, 4, or 5 substituents each independently selected from halogen, —OH, —CN, C₁₋₄ alkyl, C₁₋₄ haloalkyl, C₁₋₄ alkoxy, C₁₋₄ haloalkoxy, C₃₋₄ cycloalkyl, or (C₃₋₄ cycloalkyl)-C₁₋₄ alkyl-;
or R¹¹ and R¹² together with the nitrogen atom to which they are attached form a 4-to 8-membered heterocycloalkyl optionally substituted with 1, 2, 3, 4, or 5 substituents each independently selected from halogen, —OH, —CN, C₁₋₄ alkyl, C₁₋₄ haloalkyl, C₁₋₄ alkoxy, C₁₋₄ haloalkoxy, C₃₋₄ cycloalkyl, or (C₃₋₄ cycloalkyl)-C₁₋₄ alkyl-, wherein each of the C₁₋₄ alkyl, C₁₋₄ haloalkyl, C₁₋₄ alkoxy, C₁₋₄ haloalkoxy, C₃₋₄ cycloalkyl, or (C₃₋₄ cycloalkyl)-C₁₋₄ alkyl- is optionally substituted with 1, 2, or 3 substituents each independently selected from halogen, —OH, C₁₋₄ alkyl, C₁₋₄ haloalkyl, C₁₋₄ alkoxy, and C₁₋₄ haloalkoxy;
R¹³ is C₁₋₆ alkyl, C₃₋₆ cycloalkyl, (C₃₋₆ cycloalkyl)-C₁₋₄ alkyl-, phenyl, or phenyl-C₁₋₄ alkyl-, each of which is optionally substituted with 1, 2, 3, 4, or 5 substituents each independently selected from halogen, —OH, —CN, C₁₋₄ alkyl, C₁₋₄ haloalkyl, C₁₋₄ alkoxy, C₁₋₄ haloalkoxy, C₃₋₄ cycloalkyl, or (C₃₋₄ cycloalkyl)-C₁₋₄ alkyl-;
L¹ is C(R^{L})₂;
each R^{L} is independently H, C₁₋₂ alkyl, C₁₋₂ haloalkyl, C₁₋₂ alkoxy, or C₁₋₂ haloalkoxy;
or two R^{L} together with the carbon atom to which they are attached, optionally form C₃₋₆ cycloalkyl or a 3- to 6-membered heterocycloalkyl, each of which is optionally substituted with 1, 2, 3, or 4 substituents each independently selected from halogen, —OH, C₁₋₄ alkyl, C₁₋₄ haloalkyl, C₁₋₄ alkoxy, and C₁₋₄ haloalkoxy;
t1 is 0 or 1;
t2 is 0, 1, 2, 3, or 4;
t3 is 1 or 2; and
t4 is 0, 1, 2, 3, or 4.

2. The compound of claim 1, wherein the compound is a compound of Formula Ia: 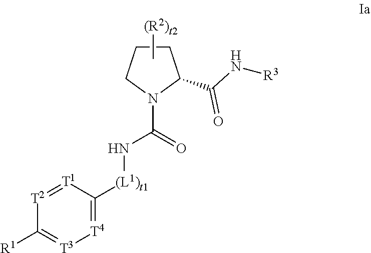
or a pharmaceutically acceptable salt thereof.

3. The compound of claim 1, wherein the compound is a compound of Formula II: 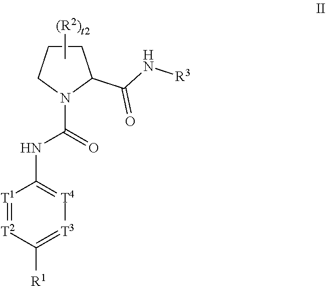
or a pharmaceutically acceptable salt thereof.

4. The compound of claim 1, wherein the compound is a compound of Formula IIa: 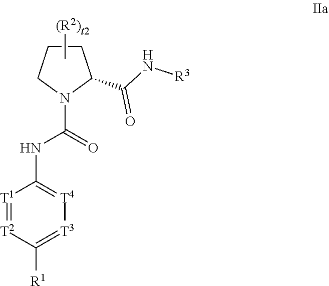
or a pharmaceutically acceptable salt thereof.

5. The compound of claim 1, wherein the compound is a compound of Formula III or IIIa: 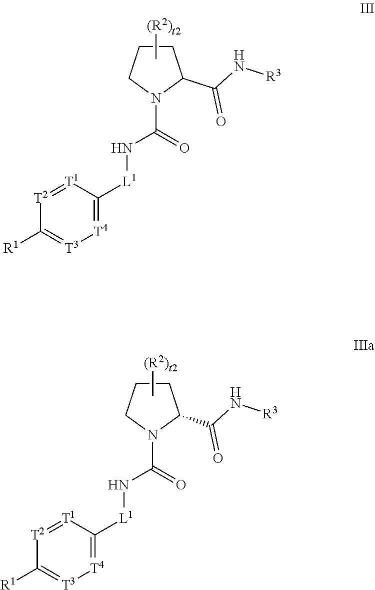
or a pharmaceutically acceptable salt thereof.

6. The compound of claim 1, wherein the compound is a compound of Formula IV or IVa: 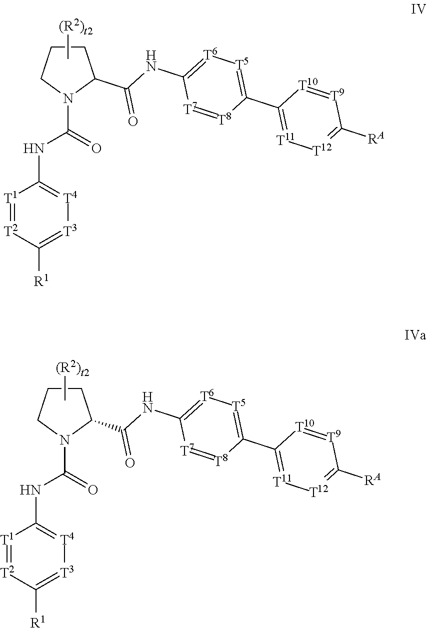
or a pharmaceutically acceptable salt thereof.

7. The compound of claim 1, wherein the compound is a compound of Formula V or Va: 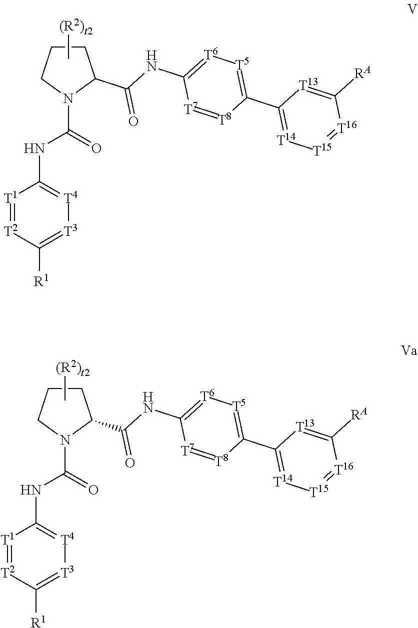
or a pharmaceutically acceptable salt thereof.

8. The compound of claim 1, wherein the compound is a compound of Formula VI or VIa: 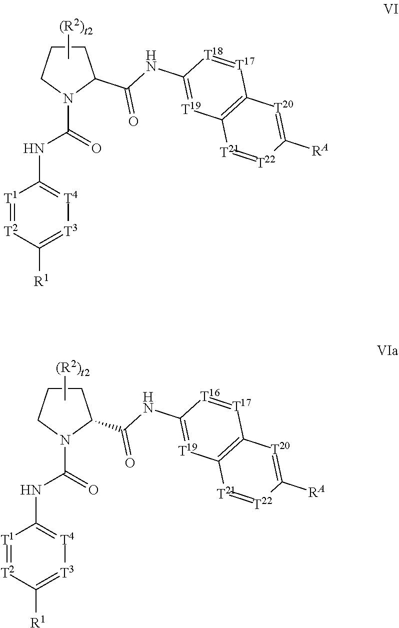
or a pharmaceutically acceptable salt thereof.

9. The compound of claim 1, wherein the compound is a compound of Formula VII or VIIa: 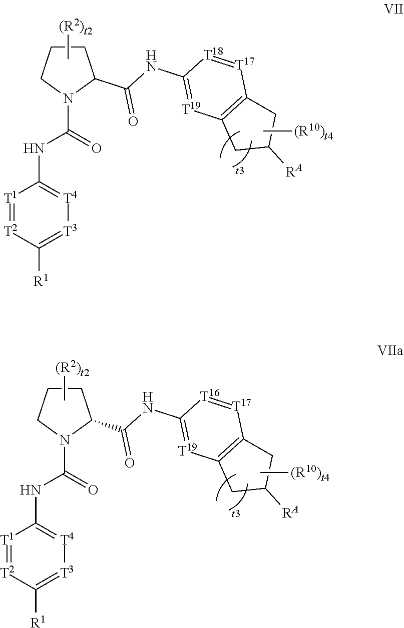
or a pharmaceutically acceptable salt thereof.

10. The compound of claim 1, wherein R¹ is cyclopropyl, cyclobutyl, R^{1a}, R^{1b}, or R^{1c}, 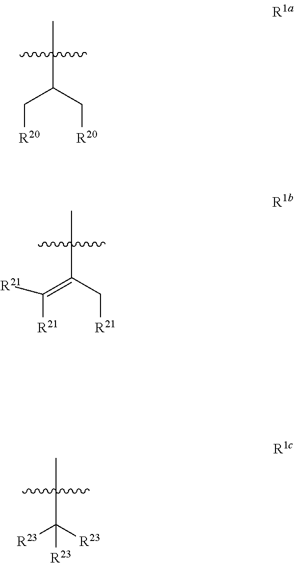
wherein each of the cyclopropyl or cyclobutyl is optionally substituted with 1, 2, 3, or 4 R^{S};
each R²⁰ is independently H, halogen, —OH, C₁₋₂ alkyl, C₁₋₂ haloalkyl, C₁₋₂ alkoxy, or C₁₋₂ haloalkoxy;
each R²¹ is independently H, C₁₋₂ alkyl, or C₁₋₂ haloalkyl;
R²² is H, halogen, C₁₋₂ alkyl, C₁₋₂ hydroxylalkyl, C₁₋₂ haloalkyl, C₁₋₂ alkoxy, or C₁₋₂ haloalkoxy;
each R²³ is independently halogen, C₁₋₂ alkyl, C₁₋₂ hydroxylalkyl, C₁₋₂ haloalkyl, C₁₋₂ alkoxy, or C₁₋₂ haloalkoxy; and
each R^{S} is independently halogen, —OH, C₁₋₂ alkyl, C₁₋₂ hydroxylalkyl, C₁₋₂ haloalkyl, C₁₋₂ alkoxy, or C₁₋₂ haloalkoxy.

11. The compound of claim 1, wherein R¹ is propan-2-yl, prop-1-en-2-yl, or cyclopropyl.

12. The compound of claim 1, wherein R¹ is propan-2-yl.

13. The compound of claim 1, wherein each of T¹, T², T³, and T⁴ is independently CR⁴.

14. The compound of claim 1, wherein one of T¹, T², T³, and T⁴ is N, and the other three are each independently CR⁴.

15. The compound of claim 1, wherein each R² is independently halogen, —OH, C₁₋₄ alkyl, C₁₋₄ hydroxylalkyl, C₁₋₄ haloalkyl, C₁₋₄ alkoxy, C₁₋₄ haloalkoxy, C₃₋₄ cycloalkyl, or (C₃₋₄ cycloalkyl)-C₁₋₄ alkyl-; and t2 is 0, 1, or 2.

16. The compound of claim 1, wherein each of T⁵, T⁶, T⁷, and T⁸ is independently CR⁵.

17. The compound of claim 1, wherein one of T⁵, T⁶, T⁷, and T⁸ is N and the other three are each independently CR⁵.

18. The compound of claim 1, wherein each of T⁹, T¹⁰, T¹¹, and T¹² is independently CR⁶.

19. The compound of claim 1, wherein one of T⁹, T¹⁰, T¹¹, and T¹² is N and the other three are each independently CR⁶.

20. The compound of claim 1, wherein each of T¹³, T¹⁴, T¹⁵, and T¹⁶ is independently CR⁷.

21. The compound of claim 1, wherein one of T¹³, T¹⁴, T¹⁵, and T¹⁶ is N and the other three are each independently CR⁷.

22. The compound of claim 1, wherein each of T¹⁷, T¹⁸, and T¹⁹ is independently CR⁸.

23. The compound of claim 1, wherein one of T¹⁷, T¹⁸, and T¹⁹ is N, and the other two are each independently CR⁸.

24. The compound of claim 1, wherein each of T²⁰, T²¹, and T²² is independently CR⁹.

25. The compound of claim 1, wherein one of T²⁰, T²¹, and T²² is N, and the other two are each independently CR⁹.

26. The compound of claim 1, wherein t3 is 2.

27. The compound of claim 1, wherein t4 is 0, 1, or 2; and each R¹⁰ is independently halogen, —OH, C₁₋₄ alkyl, C₁₋₄ hydroxylalkyl, C₁₋₄ haloalkyl, C₁₋₄ alkoxy, C₁₋₄ haloalkoxy, C₃₋₄ cycloalkyl, or (C₃₋₄ cycloalkyl)-C₁₋₄ alkyl-.

28. The compound of claim 1 wherein R^{A} is —C(═O)—OH.

29. A compound of claim 1 selected from:
5-{4-[(1-{[4-(propan-2-yl)phenyl]carbamoyl}-DL-prolyl)amino]phenyl}pyridine-2-carboxylic acid;
6-{4-[(1-{[4-(propan-2-yl)phenyl]carbamoyl}-DL-prolyl)amino]phenyl}pyridine-3-carboxylic acid;
4′-[(1-{[4-(propan-2-yl)phenyl]carbamoyl}-DL-prolyl)amino][1,1′-biphenyl]-3-carboxylic acid;
4′-[(1-{[4-(propan-2-yl)phenyl]carbamoyl}-DL-prolyl)amino][1,1′-biphenyl]-4-carboxylic acid;
4-{6-[(1-{[4-(propan-2-yl)phenyl]carbamoyl}-DL-prolyl)amino]pyridin-3-yl}benzoic acid;
3′-fluoro-4′-[(1-{[4-(propan-2-yl)phenyl]carbamoyl}-DL-prolyl)amino][1,1′-biphenyl]-4-carboxylic acid;
4′-({1-[(4-cyclopropylphenyl)carbamoyl]-DL-prolyl}amino)[1,1′-biphenyl]-4-carboxylic acid;
2-{4-[(1-{[4-(propan-2-yl)phenyl]carbamoyl}-DL-prolyl)amino]phenyl}pyrimidine-5-carboxylic acid;
6-{4-[(1-{[4-(propan-2-yl)phenyl]carbamoyl}-DL-prolyl)amino]phenyl}pyridine-2-carboxylic acid;
6-[(1-{[4-(propan-2-yl)phenyl]carbamoyl}-DL-prolyl)amino]naphthalene-2-carboxylic acid;
8-methyl-6-[(1-{[4-(propan-2-yl)phenyl]carbamoyl}-DL-prolyl)amino]quinoline-2-carboxylic acid;
4′-[(1-{[4-(prop-1-en-2-yl)phenyl]carbamoyl}-DL-prolyl)amino][1,1′-biphenyl]-4-carboxylic acid;
4′-({1-[(4-chlorophenyl)carbamoyl]-DL-prolyl}amino)[1,1′-biphenyl]-4-carboxylic acid;
4-{4-[(1-{[4-(propan-2-yl)phenyl]carbamoyl}-DL-prolyl)amino]phenyl}pyridine-2-carboxylic acid; and
3′,5′-difluoro-4′-[(1-{[4-(propan-2-yl)phenyl]carbamoyl}-DL-prolyl)amino][1,1′-biphenyl]-4-carboxylic acid,
or a pharmaceutically acceptable salt thereof.

30. A compound selected from:
5-{4-[(1-{[4-(propan-2-yl)phenyl]carbamoyl}-D-prolyl)amino]phenyl}pyridine-2-carboxylic acid;
6-{4-[(1-{[4-(propan-2-yl)phenyl]carbamoyl}-D-prolyl)amino]phenyl}pyridine-3-carboxylic acid;
4′-[(1-{[4-(propan-2-yl)phenyl]carbamoyl}-D-prolyl)amino][1,1′-biphenyl]-3-carboxylic acid;
4′-[(1-{[4-(propan-2-yl)phenyl]carbamoyl}-D-prolyl)amino][1,1′-biphenyl]-4-carboxylic acid;
4-{6-[(1-{[4-(propan-2-yl)phenyl]carbamoyl}-D-prolyl)amino]pyridin-3-yl}benzoic acid;
3′-fluoro-4′-[(1-{[4-(propan-2-yl)phenyl]carbamoyl}-D-prolyl)amino][1,1′-biphenyl]-4-carboxylic acid;
4′-({1-[(4-cyclopropylphenyl)carbamoyl]-D-prolyl}amino)[1,1′-biphenyl]-4-carboxylic acid;
2-{4-[(1-{[4-(propan-2-yl)phenyl]carbamoyl}-D-prolyl)amino]phenyl}pyrimidine-5-carboxylic acid;
6-{4-[(1-{[4-(propan-2-yl)phenyl]carbamoyl}-D-prolyl)amino]phenyl}pyridine-2-carboxylic acid;
6-[(1-{[4-(propan-2-yl)phenyl]carbamoyl}-D-prolyl)amino]naphthalene-2-carboxylic acid;
8-methyl-6-[(1-{[4-(propan-2-yl)phenyl]carbamoyl}-D-prolyl)amino]quinoline-2-carboxylic acid;
4′-[(1-{[4-(prop-1-en-2-yl)phenyl]carbamoyl}-D-prolyl)amino][1,1′-biphenyl]-4-carboxylic acid;
4′-({1-[(4-chlorophenyl)carbamoyl]-D-prolyl}amino)[1,1′-biphenyl]-4-carboxylic acid;
4-{4-[(1-{[4-(propan-2-yl)phenyl]carbamoyl}-D-prolyl)amino]phenyl}pyridine-2-carboxylic acid; and
3′,5′-difluoro-4′-[(1-{[4-(propan-2-yl)phenyl]carbamoyl}-D-prolyl)amino][1,1′-biphenyl]-4-carboxylic acid,
or a pharmaceutically acceptable salt thereof.

31. A compound of claim 1 selected from:
4′-[(1-{[4-(propan-2-yl)phenyl]carbamoyl}-DL-prolyl)amino][1,1′-biphenyl]-4-carboxylic acid;
5-{4-[(1-{[3-methyl-4-(propan-2-yl)phenyl]carbamoyl}-DL-prolyl)amino]phenyl}pyridine-2-carboxylic acid;
6-methyl-5-{4-[(1-{[4-(propan-2-yl)phenyl]carbamoyl}-DL-prolyl)amino]phenyl}pyridine-2-carboxylic acid;
3-methoxy-4′-[(1-{[4-(propan-2-yl)phenyl]carbamoyl}-DL-prolyl)amino][1,1′-biphenyl]-4-carboxylic acid;
5-{4-[(1-{[3-fluoro-4-(propan-2-yl)phenyl]carbamoyl}-DL-prolyl)amino]phenyl}-6-methylpyridine-2-carboxylic acid;
3-fluoro-4′-[(1-{[4-(propan-2-yl)phenyl]carbamoyl}-DL-prolyl)amino][1,1′-biphenyl]-4-carboxylic acid;
4-{6-[(1-{[4-(trifluoromethyl)phenyl]carbamoyl}-DL-prolyl)amino]pyridin-3-yl}benzoic acid;
4′-[(1-{[3-fluoro-4-(propan-2-yl)phenyl]carbamoyl}-DL-prolyl)amino]-3-methoxy[1,1′-biphenyl]-4-carboxylic acid;
4-{3-fluoro-5-[(1-{[4-(propan-2-yl)phenyl]carbamoyl}-DL-prolyl)amino]pyridin-2-yl}benzoic acid; and
6-methyl-5-{4-[(1-{[3-methyl-4-(trifluoromethyl)phenyl]carbamoyl}-DL-prolyl)amino]phenyl}pyridine-2-carboxylic acid,
or a pharmaceutically acceptable salt thereof.

32. A compound selected from:
4′-[(1-{[4-(propan-2-yl)phenyl]carbamoyl}-D-prolyl)amino][1,1′-biphenyl]-4-carboxylic acid;
5-{4-[(1-{[3-methyl-4-(propan-2-yl)phenyl]carbamoyl}-D-prolyl)amino]phenyl}pyridine-2-carboxylic acid;
6-methyl-5-{4-[(1-{[4-(propan-2-yl)phenyl]carbamoyl}-D-prolyl)amino]phenyl}pyridine-2-carboxylic acid;
3-methoxy-4′-[(1-{[4-(propan-2-yl)phenyl]carbamoyl}-D-prolyl)amino][1,1′-biphenyl]-4-carboxylic acid;
5-{4-[(1-{[3-fluoro-4-(propan-2-yl)phenyl]carbamoyl}-D-prolyl)amino]phenyl}-6-methylpyridine-2-carboxylic acid;
3-fluoro-4′-[(1-{[4-(propan-2-yl)phenyl]carbamoyl}-D-prolyl)amino][1,1′-biphenyl]-4-carboxylic acid;
4-{6-[(1-{[4-(trifluoromethyl)phenyl]carbamoyl}-D-prolyl)amino]pyridin-3-yl}benzoic acid;
4′-[(1-{[3-fluoro-4-(propan-2-yl)phenyl]carbamoyl}-D-prolyl)amino]-3-methoxy[1,1′-biphenyl]-4-carboxylic acid;
4-{3-fluoro-5-[(1-{[4-(propan-2-yl)phenyl]carbamoyl}-D-prolyl)amino]pyridin-2-yl}benzoic acid; and
6-methyl-5-{4-[(1-{[3-methyl-4-(trifluoromethyl)phenyl]carbamoyl}-D-prolyl)amino]phenyl}pyridine-2-carboxylic acid,
or a pharmaceutically acceptable salt thereof.

33. A compound of claim 1 selected from 4′-[(1-{[4-(trifluoromethyl)phenyl]carbamoyl}-DL-prolyl)amino][1,1′-biphenyl]-4-carboxylic acid;
5-{4-[(1-{[3-methyl-4-(trifluoromethyl)phenyl]carbamoyl}-DL-prolyl)amino]phenyl}pyridine-2-carboxylic acid;
4-{5-[(1-{[4-(propan-2-yl)phenyl]carbamoyl}-DL-prolyl)amino]pyridin-2-yl}benzoic acid;
5-{4-[(1-{[3-methyl-4-(propan-2-yl)phenyl]carbamoyl}-DL-prolyl)amino]phenyl}pyridine-2-carboxylic acid;
6-methyl-5-{4-[(1-{[4-(propan-2-yl)phenyl]carbamoyl}-DL-prolyl)amino]phenyl}pyridine-2-carboxylic acid;
3-methoxy-4′-[(1-{[4-(propan-2-yl)phenyl]carbamoyl}-DL-prolyl)amino][1,1′-biphenyl]-4-carboxylic acid;
4-{5-[(1-{[4-(trifluoromethyl)phenyl]carbamoyl}-DL-prolyl)amino]pyridin-2-yl}benzoic acid;
4′-({1-[(4-cyclobutylphenyl)carbamoyl]-DL-prolyl}amino)[1,1′-biphenyl]-4-carboxylic acid;
4-{5-fluoro-6-[(1-{[4-(trifluoromethyl)phenyl]carbamoyl}-DL-prolyl)amino]pyridin-3-yl}benzoic acid;
4-{5-fluoro-6-[(1-{[4-(propan-2-yl)phenyl]carbamoyl}-DL-prolyl)amino]pyridin-3-yl}benzoic acid;
4′-[(1-{[4-cyclopropyl-3-(trifluoromethyl)phenyl]carbamoyl}-DL-prolyl)amino][1,1′-biphenyl]-4-carboxylic acid;
5-{4-[(1-{[3-fluoro-4-(propan-2-yl)phenyl]carbamoyl}-DL-prolyl)amino]phenyl}-6-methylpyridine-2-carboxylic acid;
3-fluoro-4′-[(1-{[4-(propan-2-yl)phenyl]carbamoyl}-DL-prolyl)amino][1,1′-biphenyl]-4-carboxylic acid;
2-methoxy-4′-[(1-{[4-(propan-2-yl)phenyl]carbamoyl}-DL-prolyl)amino][1,1′-biphenyl]-4-carboxylic acid;
3-methoxy-4′-[(1-{[3-methyl-4-(propan-2-yl)phenyl]carbamoyl}-DL-prolyl)amino][1,1′-biphenyl]-4-carboxylic acid;
4-{6-[(1-{[4-(trifluoromethyl)phenyl]carbamoyl}-DL-prolyl)amino]pyridin-3-yl}benzoic acid;
4′-[(1-{[3-fluoro-4-(propan-2-yl)phenyl]carbamoyl}-DL-prolyl)amino]-3-methoxy[1,1′-biphenyl]-4-carboxylic acid;
4-{3-fluoro-5-[(1-{[4-(propan-2-yl)phenyl]carbamoyl}-DL-prolyl)amino]pyridin-2-yl}benzoic acid;
4′-{[3-methyl-1-{[4-(propan-2-yl)phenyl]carbamoyl}-DL-prolyl]amino}[1,1′-biphenyl]-4-carboxylic acid;
4′-{[(trans)-3-methyl-1-{[4-(propan-2-yl)phenyl]carbamoyl}-DL-prolyl]amino}[1,1′-biphenyl]-4-carboxylic acid;
4′-{[3-methyl-1-{[4-(propan-2-yl)phenyl]carbamoyl}-DL-prolyl]amino}[1,1′-biphenyl]-4-carboxylic acid;
4′-{[(3-trans)-3-methyl-1-{[4-(propan-2-yl)phenyl]carbamoyl}-DL-prolyl]amino}[1,1′-biphenyl]-4-carboxylic acid;
4′-{[4-methoxy-1-{[4-(propan-2-yl)phenyl]carbamoyl}-DL-prolyl]amino}[1,1′-biphenyl]-4-carboxylic acid;
4′-{[(4-cis)-4-methoxy-1-{[4-(propan-2-yl)phenyl]carbamoyl}-DL-prolyl]amino}[1,1′-biphenyl]-4-carboxylic acid;
4′-{[1-({1-[4-(propan-2-yl)phenyl]ethyl}carbamoyl)-DL-prolyl]amino}[1,1′-biphenyl]-4-carboxylic acid; and
3-[6-({1-[(4-cyclobutylphenyl)carbamoyl]-DL-prolyl}amino)pyridin-3-yl]benzoic acid,
or a pharmaceutically acceptable salt thereof.

34. A compound selected from
4′-[(1-{[4-(trifluoromethyl)phenyl]carbamoyl}-D-prolyl)amino][1,1′-biphenyl]-4-carboxylic acid;
5-{4-[(1-{[3-methyl-4-(trifluoromethyl)phenyl]carbamoyl}-D-prolyl)amino]phenyl}pyridine-2-carboxylic acid;
4-{5-[(1-{[4-(propan-2-yl)phenyl]carbamoyl}-D-prolyl)amino]pyridin-2-yl}benzoic acid;
5-{4-[(1-{[3-methyl-4-(propan-2-yl)phenyl]carbamoyl}-D-prolyl)amino]phenyl}pyridine-2-carboxylic acid;
6-methyl-5-{4-[(1-{[4-(propan-2-yl)phenyl]carbamoyl}-D-prolyl)amino]phenyl}pyridine-2-carboxylic acid;
3-methoxy-4′-[(1-{[4-(propan-2-yl)phenyl]carbamoyl}-D-prolyl)amino][1,1′-biphenyl]-4-carboxylic acid;
4-{5-[(1-{[4-(trifluoromethyl)phenyl]carbamoyl}-D-prolyl)amino]pyridin-2-yl}benzoic acid;
4′-({1-[(4-cyclobutylphenyl)carbamoyl]-D-prolyl}amino)[1,1′-biphenyl]-4-carboxylic acid;
4-{5-fluoro-6-[(1-{[4-(trifluoromethyl)phenyl]carbamoyl}-D-prolyl)amino]pyridin-3-yl}benzoic acid;
4-{5-fluoro-6-[(1-{[4-(propan-2-yl)phenyl]carbamoyl}-D-prolyl)amino]pyridin-3-yl}benzoic acid;
4′-[(1-{[4-cyclopropyl-3-(trifluoromethyl)phenyl]carbamoyl}-D-prolyl)amino][1,1′-biphenyl]-4-carboxylic acid;
5-{4-[(1-{[3-fluoro-4-(propan-2-yl)phenyl]carbamoyl}-D-prolyl)amino]phenyl}-6-methylpyridine-2-carboxylic acid;
3-fluoro-4′-[(1-{[4-(propan-2-yl)phenyl]carbamoyl}-D-prolyl)amino][1,1′-biphenyl]-4-carboxylic acid;
2-methoxy-4′-[(1-{[4-(propan-2-yl)phenyl]carbamoyl}-D-prolyl)amino][1,1′-biphenyl]-4-carboxylic acid;
3-methoxy-4′-[(1-{[3-methyl-4-(propan-2-yl)phenyl]carbamoyl}-D-prolyl)amino][1,1′-biphenyl]-4-carboxylic acid;
4-{6-[(1-{[4-(trifluoromethyl)phenyl]carbamoyl}-D-prolyl)amino]pyridin-3-yl}benzoic acid;
4′-[(1-{[3-fluoro-4-(propan-2-yl)phenyl]carbamoyl}-D-prolyl)amino]-3-methoxy[1,1′-biphenyl]-4-carboxylic acid;
4-{3-fluoro-5-[(1-{[4-(propan-2-yl)phenyl]carbamoyl}-D-prolyl)amino]pyridin-2-yl}benzoic acid;
4′-{[(3S)-3-methyl-1-{[4-(propan-2-yl)phenyl]carbamoyl}-D-prolyl]amino}[1,1-biphenyl]-4-carboxylic acid;
4′-{[(3R)-3-methyl-1-{[4-(propan-2-yl)phenyl]carbamoyl}-L-prolyl]amino}[1,1-biphenyl]-4-carboxylic acid;
4′-{[(3R)-3-methyl-1-{[4-(propan-2-yl)phenyl]carbamoyl}-D-prolyl]amino}[1,1′-biphenyl]-4-carboxylic acid;
4′-{[(3S)-3-methyl-1-{[4-(propan-2-yl)phenyl]carbamoyl}-L-prolyl]amino}[1,1′-biphenyl]-4-carboxylic acid;
4′-{[(4R)-4-methoxy-1-{[4-(propan-2-yl)phenyl]carbamoyl}-D-prolyl]amino}[1,1′-biphenyl]-4-carboxylic acid;
4′-{[1-({(1S)-1-[4-(propan-2-yl)phenyl]ethyl}carbamoyl)-D-prolyl]amino}[1,1′-biphenyl]-4-carboxylic acid; and
3-[6-({1-[(4-cyclobutylphenyl)carbamoyl]-D-prolyl}amino)pyridin-3-yl]benzoic acid,
or a pharmaceutically acceptable salt thereof.

35. A pharmaceutical composition comprising a compound of claim 1 and a pharmaceutically acceptable excipient.

36. A method for treating or preventing a condition, disease, or disorder in a patient comprising administering to the patient a compound of claim 1, wherein the condition, disease, or disorder is selected from the group consisting of diabetes, idiopathic T1D (Type 1b), latent autoimmune diabetes in adults (LADA), early-onset T2DM (EOD), youth-onset atypical diabetes (YOAD), maturity onset diabetes of the young (MODY), malnutrition-related diabetes, gestational diabetes, hyperglycemia, insulin resistance, hepatic insulin resistance, impaired glucose tolerance, diabetic neuropathy, diabetic nephropathy, kidney disease, diabetic retinopathy, adipocyte dysfunction, visceral adipose deposition, sleep apnea, obesity and related comorbidities, eating disorders, weight gain such as weight gain caused by use of other agents, overweight, excessive sugar craving, dyslipidemia, hyperinsulinemia, nonalcoholic fatty liver disease [NAFLD], cardiovascular disease, atherosclerosis (including coronary artery disease), peripheral vascular disease, hypertension, endothelial dysfunction, impaired vascular compliance, heart failure, myocardial infarction, stroke, hemorrhagic stroke, ischemic stroke, traumatic brain injury, pulmonary hypertension, restenosis after angioplasty, intermittent claudication, post-prandial lipemia, metabolic acidosis, ketosis, arthritis, osteoporosis, osteoarthritis, Parkinson's disease, left ventricular hypertrophy, peripheral arterial disease (PAD), macular degeneration, cataract, glomerulosclerosis, chronic renal failure, metabolic syndrome, syndrome X, premenstrual syndrome, angina pectoris, thrombosis, atherosclerosis, transient ischemic attacks, vascular restenosis, impaired glucose metabolism, conditions of impaired fasting plasma glucose, hyperuricemia, gout, erectile dysfunction, skin and connective tissue disorders, psoriasis, foot ulcerations, ulcerative colitis, hyper apo B lipoproteinemia, Alzheimer's Disease, schizophrenia, impaired cognition, inflammatory bowel disease, short bowel syndrome, Crohn's disease, colitis, irritable bowel syndrome, polycystic ovary syndrome (PCOS), and addiction; or a method for weight management of a human comprising administering to the human a compound of claim 1.

37. A method for modulating a glucose-dependent insulinotropic polypeptide receptor (GIPR) comprising contacting the GIPR with a compound of claim 1.
